(19) United States
(12) Patent Application Publication
(10) Pub. No.: US 2024/0002497 A1
(43) Pub. Date: Jan. 4, 2024

(54) NAV1.7 BINDERS

(71) Applicants: MERCK SHARP & DOHME LLC, Rahway, NJ (US); MERCK SHARP & DOHME RESEARCH GMBH, Lucerne (CH)

(72) Inventors: Pravien Damitha Abeywickrema, Blue Bell, PA (US); Erik Depla, Ghent (BE); Bruno Dombrecht, Heusden (BE); Daniel M. Gorman, Redwood City, CA (US); Andrea K. Houghton, Doylestown, PA (US); Robert A. Kastelein, Portola Valley, CA (US); Richard L. Kraus, Chalfont, PA (US); John Majercak, Wayne, PA (US); Kalyan Pande, San Francisco, CA (US); Sujata Sharma, Eagleville, PA (US)

(73) Assignees: MERCK SHARP & DOHME LLC, Rahway, NJ (US); MERCK SHARP & DOHME RESEARCH GMBH, Lucerne (CH)

(21) Appl. No.: 18/252,428

(22) PCT Filed: Nov. 18, 2021
(86) PCT No.: PCT/US2021/059842

§ 371 Date: May 10, 2023

Related U.S. Application Data
(60) Provisional application No. 63/271,963, filed on Oct. 26, 2021.
(60) Provisional application No. 63/115,878, filed on Nov. 19, 2020.

(51) Int. Cl. C07K 16/28; A61P 23/00

(52) U.S. Cl. CPC C07K 16/28; A61P 23/00; A61K 2039/505

(57) ABSTRACT

Antibodies and antigen-binding fragments thereof that bind the human voltage-gated sodium channel Nav1.7α protein subunit (Nav1.7 binders) are described. In particular embodiments, the Nav1.7 binders comprise a heavy-chain immunoglobulin single variable domain (ISVD or VHH).

### BACKGROUND OF THE INVENTION

### Field of the Invention

The present invention relates to antibodies and antigen-binding fragments thereof that bind the human voltage-gated sodium channel Nav1.7a protein subunit (Nav1.7 binders). In particular, the present invention relates to Nav1.7 binders comprising a heavy-chain immunoglobulin single variable domain (ISVD or VHH).

### Description of Related Art

Nav1.7α subunit belongs to a family of nine voltage-gated sodium channels that play crucial roles in the electrical conductance of skeletal muscles (Nav1.4α), cardiac muscles (Nav1.5α), central (Nav1.1α, Nav1.2α, Nav1.3α and Nav1.6α) and peripheral (Nav1.1α, Nav1.6α, Nav1.7α, Nav1.8α and Nav1.9α) neurons. Nav1.7α is mainly expressed on different types of afferent fibres of the peripheral nervous system and is essential to the firing of action potentials by boosting subthreshold stimuli (Dib-Hajj & Waxman 2015 Pain 156: 2406). Extensive genetic evidence in mice and men suggests that Nav1.7 is necessary and non-redundant in pain and olfactory pathways (reviewed by Dib-Hajj et al. 2013 Nat Rev Neurosci. 14: 49). Interestingly, a large and diverse body of naturally occurring toxins acts on voltage-gated sodium channels, including Nav1.7α (reviewed by Deuis et al., 2017 Neuropharmaco DOI10.1016/j.neuropharm.2017.04.014). Nav1.7α has been one of the most hotly pursued targets in the field of chronic pain where there is a large unmet need (reviewed by de Lera Ruiz & Kraus 2015 J Med Chem 58: 7093). Marketed painkillers like local anaesthetics effectively target voltage-gated sodium channels but suffer from undesired side effects prohibiting widespread use in chronic pain indications. Recent efforts to generate more selective Nav1.7α small molecule inhibitors or modified peptide toxins have failed to deliver a marketed drug so far. Attempts to generate selective anti-Nav1.7α biologicals were not reproducible (Lee et al 2014 Cell 157:1393; Liu et al. 2016 F1000Res 5:2764; and many patents).

Four consecutive similar domains, DI to DIV (FIG. 1), make up the nearly 2,000 amino acids large Nav1.7α channel. Each domain has six transmembrane helices (51 to S6 in bottom panel FIG. 1 connected by extracellular loops (ECLs) and intracellular loops (ICLs) (respectively solid and dotted lines in bottom panel FIG. 1. Two small (S1-52 and S3-S4) and one larger (S5-S6) ECL per domain make up the limited extracellular surface of the channel accessible to biologicals (cytoplasmic membrane is marked by dotted lines in top right panel in FIG. 1). The different domains are connected by ICLs (S6-S1) and both N- and C-terminal ends reside at the cytoplasmic side of the channel (marked respectively by N and C in bottom panel FIG. 1). Each domain consists of a voltage sensor domain (VSD; S1-S4) and ion-conducting pore domain (PD; S5-S6) arranged such that the VSD of each domain is closest to the PD of the following domain, in a clockwise orientation. The central Na⁺-conducting pore of the channel (marked by a star in bottom panel 1) is formed by the PDs and their ECLs that line the cavity. FIG. 32 is a schematic representation of Nav1.7α.

Voltage-gated sodium channels may interact with different Navβ-subunits (Navβ1 to Navβ4) that among other things can modulate the channels' electrophysiological properties and cell surface expression levels (reviewed by Winters & Isom 2016 Current Topics in Membranes 78: 315). The bottom panel of FIG. 1 depicts suggested interaction sites for three different Navβ-subunits, according to recent findings (Das et al. 2016 eLIFE 5:e10960; Zhu et al. 2017 J Gen Physiol 149: 813; Yan et al. 2017 Cell 170: 470).

A detailed sequence comparison of the different ECLs of huNav1.7α to their ortholog and paralog counterparts can be found in FIGS. 2A-2B. Different splice variants of Nav1.7α exist that through interaction with Navβ1 impact on the electrophysiological properties of the channel (Chatelier et al. 2008 J Neurophysiol 99: 2241; Farmer et al. 2012 PLoS ONE 7: e41750). The major technical drawbacks of Nav1.7α as a target for biologicals are its poor cell surface expression level combined with a limited accessibility to the extracellular surface.

### BRIEF SUMMARY OF THE INVENTION

The present invention provides Nav1.7 binders, which are immunoglobulin single variable domains (ISVDs) that bind and inhibit Nav1.7α channels with exquisite selectivity over other Nav channel paralogs. The Nav1.7 binders may be useful for preparing formulations for treating chronic pain or pain.

The present invention provides Nav1.7 binders that bind to a human voltage-gated sodium channel Nav1.7α protein subunit (human NaV1.7a subunit) between amino acids 272 and 331 of the human NaV1.7α subunit Domain 1 S5-S6 loop, wherein the human NaV1.7α subunit comprises the amino acid sequence set forth in SEQ ID NO: 1. In particular embodiments, the Nav1.7 binder contacts amino acids F276, R277, E281, and V331 of the human NaV1.7α subunit, which in particular embodiments, binds to the human NaV1.7α subunit with lower affinity than to human NaV1.7α subunit lacking such substitutions. In certain embodiments, the Nav1.7 binder further is capable of binding a rhesus monkey human NaV1.7α subunit with a lower affinity than it binds to the human NaV1.7α subunit.

The Nav1.7 binder is an antibody or an antibody fragment, which in specific embodiments is a heavy chain antibody or an ISVD. In particular embodiments, the heavy chain antibody is a camelid antibody and the ISVD is a VHH.

In particular embodiments, the Nav1.7 binder comprises (a) a complementarity determining region (CDR) 1, CDR1, comprising the amino acid sequence set forth in SEQ ID NO: 247, a CDR2 comprising the amino acid sequence set forth in SEQ ID NO: 248, and a CDR3 comprising the amino acid sequence set forth in SEQ ID NO: 249; or (b) a CDR1 comprising the amino acid sequence set forth in SEQ ID NO: 250, a CDR2 comprising the amino acid sequence set forth in SEQ ID NO: 251, and a CDR3 comprising the amino acid sequence set forth in SEQ ID NO: 252; or (c) a CDR1 comprising the amino acid sequence set forth in SEQ ID NO: 253, a CDR2 comprising the amino acid sequence set forth in SEQ ID NO: 254, and a CDR3 comprising the amino acid sequence SRY; or (d) a CDR1 comprising the amino acid sequence set forth in SEQ ID NO: 256, a CDR2 comprising the amino acid sequence set forth in SEQ ID NO: 257, and a CDR3 comprising the amino acid sequence set forth in SEQ ID NO: 258; or (e) a CDR1 comprising the amino acid sequence set forth in SEQ ID NO: 259, a CDR2 comprising the amino acid sequence set forth in SEQ ID NO: 260, and a CDR3 comprising the amino acid sequence set forth in SEQ ID NO: 261; or (f) a CDR1 comprising the amino acid sequence set forth in SEQ ID NO: 262, a CDR2 comprising the amino acid sequence set forth in SEQ ID NO: 263, and a CDR3 comprising the amino acid sequence set forth in SEQ ID NO: 264; or (g) a CDR1 comprising the amino acid sequence set forth in SEQ ID NO: 196, a CDR2 comprising the amino acid sequence set forth in SEQ ID NO: 198, and a CDR3 comprising the amino acid sequence set forth in SEQ ID NO: 200; or (h) a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 206; or (i) a CDR1 comprising the amino acid sequence set forth in SEQ ID NO: 207, a CDR2 comprising the amino acid sequence set forth in SEQ ID NO: 213, and a CDR3 comprising the amino acid sequence set forth in SEQ ID NO: 219; or (j) a CDR1 comprising the amino acid sequence set forth in SEQ ID NO: 221, a CDR2 comprising the amino acid sequence set forth in SEQ ID NO: 223, and a CDR3 comprising the amino acid sequence set forth in SEQ ID NO: 225.

In a further embodiment, the Nav1.7 binder comprises (a) a CDR1 comprising the amino acid sequence set forth in SEQ ID NO: 196 or SEQ ID NO: 197; a CDR2 comprising the amino acid sequence set forth in SEQ ID NO: 198 or SEQ ID NO: 199; and, a CDR3 comprising the amino acid sequence set forth in SEQ ID NO: 200; or (b) a CDR1 comprising the amino acid sequence set forth in SEQ ID NO: 201; a CDR2 comprising the amino acid sequence set forth in SEQ ID NO: 202, SEQ ID NO: 203, SEQ ID NO: 204, or SEQ ID NO: 205; and, a CDR3 comprising the amino acid sequence set forth in SEQ ID NO: 206; or (c) a CDR1 comprising the amino acid sequence set forth in SEQ ID NO: 207, SEQ ID NO: 208, SEQ ID NO: 209, SEQ ID NO: 210, SEQ ID NO: 211, or SEQ ID NO: 212; a CDR2 comprising the amino acid sequence set forth in SEQ ID NO: 213, SEQ ID NO: 214, SEQ ID NO: 215, SEQ ID NO: 216, SEQ ID NO: 217, or SEQ ID NO: 218; and a CDR3 comprising the amino acid sequence set forth in SEQ ID NO: 219; or (d) a CDR1 comprising the amino acid sequence set forth in SEQ ID NO: 201 or SEQ ID NO: 222; a CDR2 comprising the amino acid sequence set forth in SEQ ID NO: 223 or SEQ ID NO: 224; and a CDR3 comprising the amino acid sequence set forth in SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, or SEQ ID NO: 233; or (e) a CDR1 comprising the amino acid sequence set forth in SEQ ID NO: 201; a CDR2 comprising the amino acid sequence set forth in SEQ ID NO: 205; and, a CDR3 comprising the amino acid sequence set forth in SEQ ID NO: 206; or (0 a CDR1 comprising the amino acid sequence set forth in SEQ ID NO: 211; a CDR2 comprising the amino acid sequence set forth in SEQ ID NO: 215; and, a CDR3 comprising the amino acid sequence set forth in SEQ ID NO: 219; or (g) a CDR1 comprising the amino acid sequence set forth in SEQ ID NO: 222; a CDR2 comprising the amino acid sequence set forth in SEQ ID NO: 223; and, a CDR3 comprising the amino acid sequence set forth in SEQ ID NO: 233.

In a further embodiment the Nav1.7 binder comprises (a) an amino acid sequence selected from the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81; or (b) an amino acid sequence selected from the group consisting of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, and SEQ ID NO: 97; or (c) an amino acid sequence selected from the group consisting of SEQ ID NO: 98, SEQ ID NO: 99, SEQ ID NO: 100, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, and SEQ ID NO: 153; or (d) an amino acid sequence selected from the group consisting of SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, and SEQ ID NO: 195.

In particular embodiments, the Nav1.7 binder comprises a C-terminal alanine residue.

In particular embodiments, the Nav1.7 binder is conjugated to a half-life extender, which in certain embodiments is a human serum albumin (HSA) binder or the crystallizable fragment (Fc) of an antibody. HSA binders include but are not limited ALB11002 or ALB00223. In particular embodiments, the Nav1.7 binder is conjugated to is polyethylene glycol, which provides half-life extension.

The present invention further provides for use of a Nav1.7 binder disclosed herein for the manufacture of a medicament for the treatment of chronic pain.

The present invention further provides for use of a Nav1.7 binder disclosed herein for the treatment of chronic pain.

The present invention further provides a method for treating an individual with chronic pain comprising administering to the individual a therapeutically effective amount of a Nav1.7 binder disclosed herein to treat the chronic pain. The individual may be a human patient in need of pain relief. The human patient may be treated in a hospital setting or in an out-patient setting. The Nav1.7 binder may be administered by syringe, autoinjector, dose-settable delivery device, or the like.

The present invention further provides a composition comprising a Nav1.7 binder disclosed herein and a pharmaceutically acceptable carrier.

The present invention further provides a nucleic acid molecule encoding the Nav1.7 binder disclosed herein. In a further embodiment the nucleic acid molecule encoding the Nav1.7 binder comprises a nucleotide sequence selected from the group consisting of nucleotide sequences set forth in SEQ ID NO: 273-283. In a further embodiment the nucleic acid molecule encoding the Nav1.7 binder comprises a nucleotide sequence selected from the group consisting of nucleotide sequences set forth in SEQ ID NO: 284-421.

The present invention further provides a vector comprising the nucleic acid molecule encoding a Nav.7 binder. The present invention further provides a host cell comprising a nucleic acid molecule encoding a Nav1.7 binder disclosed herein.

The present invention further provides a method for producing a Nav1.7 binder disclosed herein comprising: (a) providing a host cell comprising a nucleic acid molecule encoding a Nav1.7 binder disclosed herein or a vector comprising a nucleic acid molecule encoding the Nav1.7 binder disclosed herein; (b) cultivating the host cell in a medium under conditions suitable for expression of the Nav1.7 binder by the host cell; and (c) isolating the Nav1.7 binder from the medium to provide the Nav1.7 binder.

The present invention further provides a Navβ1 binder comprising (a) a first immunoglobulin single variable domain (ISVD) comprising three complementarity determining regions (CDRs) wherein CDR1 comprises the amino acid sequence set forth in SEQ ID NO: 425, CDR2 comprises the amino acid sequence set forth in SEQ ID NO: 426, and CDR3 comprises the amino acid sequence set forth in SEQ ID NO: 427; or (b) a second ISVD comprising three CDRs wherein CDR1 comprises the amino acid sequence set forth in SEQ ID NO: 437, CDR2 comprises the amino acid sequence set forth in SEQ ID NO: 438, and CDR3 comprise the amino acid sequence set forth in SEQ ID NO: 439.

Ina further embodiment of the Navβ1 binder, the first ISVD comprises the amino acid sequence set forth in SEQ ID NO: 411 and the second ISVD comprises the amino acid sequence set forth in SEQ ID NO: 415. In a further embodiment, the N-terminal amino acid of the first ISVD or the second ISVD is linked to the C-terminal amino acid of a Nav1.7 binder of claim 1 by a peptide or polypeptide linker or the N-terminal amino acid of the Nav1.7 binder of claim 1 is linked to the C-terminal amino acid of the first ISVD or the second ISVD by a peptide or polypeptide linker.

In further embodiments of the Navβ1 binder, the peptide or polypeptide linker comprises any combination of glycine and serine amino acids up to 40 amino acids. In a further embodiment of the Navβ1 binder, the peptide or polypeptide linker comprises an amino acid sequence comprising GGGGS (SEQ ID NO: 246)) n wherein n is 1, 2, 3,4, 5, 6, 7, 8, 9 or 10. In a particular embodiment, the polypeptide linker comprises the amino acid sequence set forth in SEQ ID NO: 463.

The present invention further provides a nucleic acid molecule encoding a Navβ1 binder disclosed herein. In a further embodiment, the Navβ1 binder comprises a nucleotide sequence selected from the group consisting of nucleotide sequences set forth in SEQ ID NO: 456 and 461.

The present invention further provides a vector comprising the nucleic acid molecule encoding a Navβ1 binder disclosed herein. The present invention further provides a host cell comprising a nucleic acid molecule encoding a Navβ1 binder disclosed herein.

The present invention further provides a method for producing a Navβ1 binder disclosed herein comprising: (a) providing a host cell comprising a nucleic acid molecule encoding a Navβ1 binder disclosed herein or a vector comprising a nucleic acid molecule encoding the Navβ1 binder disclosed herein; (b) cultivating the host cell in a medium under conditions suitable for expression of the Navβ1 binder by the host cell; and (c) isolating the Navβ1 binder from the medium to provide the Navβ1 binder.

The present invention further provides a Navβ2 binder comprising (a) a first immunoglobulin single variable domain (ISVD) comprising three complementarity determining regions (CDRs) wherein CDR1 comprises the amino acid sequence set forth in SEQ ID NO: 422, a CDR2 comprising the amino acid sequence set forth in SEQ ID NO: 423, and a CDR3 comprising the amino acid sequence set forth in SEQ ID NO: 424; (b) a second ISVD comprising three CDRs wherein CDR1 comprises the amino acid sequence set forth in SEQ ID NO: 428, a CDR2 comprising the amino acid sequence set forth in SEQ ID NO: 429, and a CDR3 comprising the amino acid sequence set forth in SEQ ID NO: 430; (c) a third ISVD comprising three CDRs wherein CDR1 comprises the amino acid sequence set forth in SEQ ID NO: 431, a CDR2 comprising the amino acid sequence set forth in SEQ ID NO: 432, and a CDR3 comprising the amino acid sequence set forth in SEQ ID NO: 433; or (d) a fourth ISVD comprising three CDRs wherein CDR1 comprises the amino acid sequence set forth in SEQ ID NO: 434, a CDR2 comprising the amino acid sequence set forth in SEQ ID NO: 435, and a CDR3 comprising the amino acid sequence set forth in SEQ ID NO: 436.

In a further embodiment of the Navβ2 binder, the first ISVD comprises the amino acid sequence set forth in SEQ ID NO: 410, the second ISVD comprises the amino acid sequence set forth in SEQ ID NO: 412, the third ISVD comprises the amino acid sequence set forth in SEQ ID NO: 413, and the fourth ISVD comprises the amino acid sequence set forth in SEQ ID NO: 414.

In a further embodiment of the Navβ2 binder, the N-terminal amino acid of the first ISVD, the second ISVD, the third ISVD, or the fourth ISVD is linked to the C-terminal amino acid of a Nav1.7 binder of claim 1 by a peptide or polypeptide linker or the N-terminal amino acid of the Nav1.7 binder of claim 1 is linked to the C-terminal amino acid of the first ISVD, the second ISVD, the third ISVD, or the fourth ISVD by a peptide or polypeptide linker.

In a further embodiment, the peptide or polypeptide linker comprises any combination of glycine and serine amino acids up to 40 amino acids. In further embodiments, the peptide or polypeptide linker comprises an amino acid sequence comprising GGGGS (SEQ ID NO: 246)) n wherein n is 1, 2, 3,4, 5, 6, 7, 8, 9 or 10. In particular embodiments, the polypeptide linker comprises the amino acid sequence set forth in SEQ ID NO: 463.

The present invention further provides a nucleic acid molecule encoding a Navβ2 binder disclosed herein. In a further embodiment, the Navβ1 binder comprises a nucleotide sequence selected from the group consisting of nucleotide sequences set forth in SEQ ID NO: 456, 458, 459, and 460.

The present invention further provides a vector comprising the nucleic acid molecule encoding a Navβ1 binder disclosed herein. The present invention further provides a host cell comprising a nucleic acid molecule encoding a Navβ1 binder disclosed herein.

The present invention further provides a method for producing a Navβ1 binder disclosed herein comprising: (a) providing a host cell comprising a nucleic acid molecule encoding a Navβ1 binder disclosed herein or a vector comprising a nucleic acid molecule encoding the Navβ1 binder disclosed herein; (b) cultivating the host cell in a medium under conditions suitable for expression of the Navβ1 binder by the host cell; and (c) isolating the Navβ1 binder from the medium to provide the Navβ1 binder.

The present invention further provides a Nav1.7-Navβ bispecific binder comprising a Nav1.7 binder as disclosed herein and a Navβ binder selected from the group consisting of the Navβ1 binder or Navβ2 binder as disclosed herein.

In further embodiments of the Nav1.7-Navβ bispecific binder, (a) the Nav1.7 binder comprises: (i) an amino acid sequence selected from the group consisting of SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55; (ii) an amino acid sequence selected from the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81; or (iii) an amino acid sequence selected from the group consisting of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, and SEQ ID NO: 97; or (iv) an amino acid sequence selected from the group consisting of SEQ ID NO: 98, SEQ ID NO: 99, SEQ ID NO: 100, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, and SEQ ID NO: 153; or (v) an amino acid sequence selected from the group consisting of SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, and SEQ ID NO: 195; (b) the Navβ1 binder comprises an amino acid sequence selected from the group consisting of SEQ ID NO: 411 and SEQ ID NO: 415; and, (c) the Navβ2 binder comprises an amino acid sequence selected from the group consisting of SEQ ID NO: 410, SEQ ID NO: 412, SEQ ID NO: 413, and SEQ ID NO: 414.

The present invention further provides a Nav1.7-Navβ bispecific binder wherein the Nav1.7-Navβ bispecific binder is linked to a half-life extender.

The present invention further provides a Nav1.7-Navβ bispecific binder disclosed herein wherein the half-life extender is a human serum albumin (HSA) binder or HC constant domain or crystallizable fragment (Fc domain). The present invention further provides a Nav1.7-Navβ bispecific binder disclosed herein wherein the Nav1.7-Navβ bispecific binder comprises a C-terminal alanine residue.

The present invention further provides a composition comprising a Nav1.7-Navβ bispecific binder disclosed herein and a pharmaceutically acceptable carrier.

The present invention further provides for the use of a Nav1.7-Navβ bispecific binder disclosed herein for the manufacture of a medicament for the treatment of chronic pain.

The present invention further provides a Nav1.7-Navβ bispecific binder disclosed herein or a composition comprising said Nav1.7-Navβ bispecific binder for the treatment of chronic pain.

The present invention further provides a method for treating an individual with chronic pain comprising administering to the individual a therapeutically effective amount of the Nav1.7-Navβ bispecific binder disclosed herein or a composition comprising said Nav1.7-Navβ bispecific binder to treat the chronic pain.

The present invention further provides a nucleic acid molecule encoding a Nav1.7-Navβ bispecific binder comprising a nucleic acid molecule encoding a Nav1.7 binder disclosed herein and a Navβ1 or Navβ2 binder disclosed herein. In a further embodiment, the nucleic acid molecule encoding the Nav1.7 binder comprises a nucleotide sequence selected from the group consisting of nucleotide sequences set forth in SEQ ID NO: 273-283, the Navβ1 binder comprises a nucleotide sequence selected from the group consisting of nucleotide sequences set forth in SEQ ID NO: 457 and 461, and Navβ2 binder comprises a nucleotide sequence selected from the group consisting of nucleotide sequences set forth in SEQ ID NO: 456, 458, 459, and 460. In a further embodiment, the nucleic acid molecule encoding the Nav1.7 binder comprises a nucleotide sequence selected from the group consisting of nucleotide sequences set forth in SEQ ID NO: 284-421, the Navβ1 binder comprises a nucleotide sequence selected from the group consisting of nucleotide sequences set forth in SEQ ID NO: 457 and 461, and Navβ2 binder comprises a nucleotide sequence selected from the group consisting of nucleotide sequences set forth in SEQ ID NO: 456, 458, 459, and 460.

The present invention further provides a vector comprising the nucleic acid molecule encoding a Nav1.7-Navβ bispecific binder disclosed herein. The present invention further provides a host cell comprising a nucleic acid molecule encoding a Nav1.7-Navβ bispecific binder disclosed herein.

The present invention further provides a method for producing a Nav1.7-Navβ bispecific binder disclosed herein comprising: (a) providing a host cell comprising a nucleic acid molecule encoding a Nav1.7-Navβ bispecific binder disclosed herein or a vector comprising a nucleic acid molecule encoding the Nav1.7-Navβ bispecific binder disclosed herein; (b) cultivating the host cell in a medium under conditions suitable for expression of the Nav1.7-Navβ bispecific binder by the host cell; and (c) isolating the Nav1.7-Navβ bispecific binder from the medium to provide the Nav1.7-Navβ bispecific binder.

The present invention further provides a Nav1.7 binder, Navβ1 binder, or Navβ2 binder comprising an amino acid sequence disclosed in Table 56. The present invention further provides a nucleic acid molecule encoding a Nav1.7 binder, Navβ1 binder, or Navβ2 binder and comprising a nucleotide sequence having at least 80, 90%, 95%, or 100% identity to a nucleotide sequence disclosed in Table 56 provided the amino acid sequence encoded by the nucleotide sequence is disclosed in Table 56. The present invention further provides a Nav1.7-Navβ bispecific binder comprising an amino acid sequence disclosed in Table 56 or comprised of a Nav1.7 binder and at least one Navβ binder selected from Navβ1 binder and Navβ2 binder, each comprising an amino acid sequence disclosed in Table 56. The present invention further provides a nucleic acid molecule comprising a nucleotide sequence encoding a Nav1.7-Navβ bispecific binder wherein the nucleotide sequence has at least 80, 90%, 95%, or 100% identity to a nucleotide sequence disclosed in Table 56 provided the nucleotide sequence encodes an amino acid sequence disclosed in Table 56.

### BRIEF DESCRIPTION OF THE DRAWINGS

FIG. 1 shows the proposed structure of Nav1.7α. Drawing shows a huNav1.7α model viewed from top/extracellular (top left panel) and side through cytoplasmic membrane (top right panel). Nav1.7α structural topology viewed from extracellular side (bottom panel) shown with β1, β2, and β3 subunits.

FIG. 2A and FIG. 2B together show sequence comparisons of huNav1.7α to paralogs and orthologs (based on sequences listed in the Table 41).

FIG. 3A shows the binding of ISVDs F103262CO2, F0103265B04, F0103262B06, F0103265A11 to huNav1.7α+β1−β2−β3. MFI=median fluorescence intensity; IRR=irrelevant control ISVD; a-FLAG is a detection moiety.

FIG. 3B shows the binding of ISVD F0103362B08 to huNav1.7α++β1−β2−β3. MFI=median fluorescence intensity; a-FLAG is a detection moiety.

FIG. 3C shows the binding of ISVDs F103262CO2, F0103265B04, F0103262B06, F0103265A11 to huNav1.7α+β1. MFI=median fluorescence intensity; IRR=irrelevant control ISVD; a-FLAG is a detection moiety.

FIG. 3D shows the binding of ISVDs F103262CO2, F0103265B04, F0103262B06, F0103265A11 to huNav1.5α−β1−β2−β3. MFI=median fluorescence intensity; IRR=irrelevant control ISVD; a-FLAG is a detection moiety.

FIG. 3E shows the binding of ISVDs F0103265B04 and F0103262B08, to huNav1.7+β1−β2−β3. MFI=median fluorescence intensity; IRR=irrelevant control ISVD; a-FLAG is a detection moiety.

FIG. 3F shows the binding of ISVDs F0103265B04 and F0103262B08, to huNav1.5α−β1−β2−β3. MFI=median fluorescence intensity; IRR=irrelevant control ISVD; a-FLAG is a detection moiety.

FIG. 3G shows the binding of ISVDs F103262CO2, F0103265B04, F0103262B06, F0103265A11 to huNav157chimera14−β1−β2−β3. MFI=median fluorescence intensity; IRR=irrelevant control ISVD; a-FLAG is a detection moiety.

FIG. 3H shows the binding of ISVDs F0103265B04 and F0103262B08, to huNav1.7α+β1. MFI=median fluorescence intensity; IRR=irrelevant control ISVD; a-FLAG is a detection moiety.

FIG. 3I shows the binding of ISVDs F0103265B04 and F0103262B08, to huNav157chimera14−β1−β2−β3. MFI=median fluorescence intensity; IRR=irrelevant control ISVD; a-FLAG is a detection moiety.

FIG. 4 shows a sequence alignment of functional Nav1.7α+selective ISVDs compared to the human VH3-JH consensus sequence (SEQ ID NO: 57). Residues identical to the human VH3-JH consensus are shown by dots. CDRs are highlighted. The amino acid sequences for the ISVDs are F0103265B04 (SEQ ID NO: 49); F0103275B05 (SEQ ID NO: 50), F0103387G04 (SEQ ID NO: 52); F0103265A11 (SEQ ID NO: 48); F0103387G05 (SEQ ID NO: 53); F0103362B08 (SEQ ID NO: 51).

FIG. 5 shows screening of the F0103275B05 (275B05) stage I affinity maturation library in binding fluorescence-activated cell sorting (FACS) on huNav1.7α and rhNav1.7α.

FIG. 6 shows screening of the F0103275B05 (275B05) stage II affinity maturation library in binding FACS on huNav1.7α and rhNav1.7α.

FIG. 7A shows a schematic for a single pulse electrophysiology protocol.

FIG. 7B shows a schematic for a two pulse electrophysiology protocol.

FIG. 8 shows screening of the F0103265A11 (265A11) stage I affinity maturation library in binding FACS on huNav1.7α and rhNav1.7α.

FIG. 9 shows screening of the F0103265A11 (265A11) stage II affinity maturation library in binding FACS on huNav1.7α and rhNav1.7α.

FIG. 10 shows screening of the F0103265B04 (265B04) stage I affinity maturation library in binding FACS on huNav1.7α and rhNav1.7α.

FIG. 11 shows screening of the F0103387G05 (387G05) stage I affinity maturation library in binding FACS on huNav1.7α and rhNav1.7α.

FIG. 12 shows screening of the F0103362B08 (362B08) stage I affinity maturation library in binding FACS on huNav1.7α and rhNav1.7α.

FIG. 13 shows screening of the F0103464B09 (464B09) stage I affinity maturation library in binding FACS on huNav1.7α and rhNav1.7α.

FIG. 14 shows screening of the F0103464B09 (464B09) stage II affinity maturation library in binding FACS on huNav1.7α and rhNav1.7α.

FIG. 15A shows competition FACS of extracellular anti-Nav1.7α ISVDs vs. F0103265B04 on stable HEK cell lines expressing huNav1.7+β1−β2−β3.

FIG. 15B shows competition FACS of extracellular anti-Nav1.7α ISVDs vs. F0103265B04 on stable HEK cell lines expressing huNav1.7α+β1−β2−β3.

FIG. 15C shows competition FACS of extracellular anti-Nav1.7α ISVDs vs. F0103275B05(N93R) on stable CHO cell lines expressing huNav1.7α+β1−β2−β3.

FIG. 15D shows competition FACS of extracellular anti-Nav1.7α ISVDs vs. F0103275B05(N93R) on stable CHO cell lines expressing rhNav1.7α+β1−β2−β3.

FIG. 16 shows a schematic overview of huNav1.7α+huNav1.5α (huNav157) chimeras.

FIG. 17A, FIG. 17B, and FIG. 17C together show epitope mapping FACS of extracellular anti-Nav1.7α ISVDs (1 μM) on transiently transfected cells expressing huNav157+β1−β2−β3 chimeras 1, 2, 3, or 4 (huNav157chim1, huNav157chim2, huNav157chim3, or huNav157chim4, respectively) compared to cells expressing huNav1.7α+β1−β2−β3.

FIG. 18A, FIG. 18B, and FIG. 18C together show epitope mapping FACS of extracellular anti-Nav1.7α ISVDs (1 μM) on transiently transfected cells expressing huNav157+β1−β2−β3 chimeras 5, 6, 7, or 8 (huNav157chim5, huNav157chim6, huNav157chim7, or huNav157chim8, respectively) compared to cells expressing huNav1.7α+β1−β2−β3.

FIG. 19A and FIG. 19B together show epitope mapping FACS of extracellular anti-Nav1.7α ISVDs (1 μM) on transiently transfected cells expressing huNav157+β1−β2−β3 chimeras 9 or 12 (huNav157chim9 or huNav157chim12, respectively) compared to cells expressing huNav1.7α+β1−β2−β3.

FIG. 20A and FIG. 20B together show epitope mapping FACS of extracellular anti-Nav1.7α ISVDs (1 μM) on transiently transfected cells expressing huNav157+β1−β2−β3 chimeras 22 or 18 (huNav157chim22 or huNav157chim18, respectively) compared to cells expressing huNav1.7α+β1−β2−β3.

FIG. 21A and FIG. 21B together show shows epitope mapping FACS of extracellular anti-Nav1.7α ISVDs (1 μM) on transiently transfected cells expressing huNav1.7+β1−β2−β3, rhNav1.7+β1−β2−β3 or huNav1.7(N146S, V1941, F276V, R277Q, E281V, V331M, E504D, D507E, S508N, N533S)−β1−β2−β3.

FIG. 22A shows binding FACS of extracellular anti-Nav1.7α ISVDs on stable huNav1.7α-rhNav1.7α chimera cell line CHO FlpIn huNav1.7α+β1−β2−β3.

FIG. 22B shows binding FACS of extracellular anti-Nav1.7α ISVDs on stable huNav1.7α-rhNav1.7α chimera cell line CHO FlpIn RhNav1.7α+β1−β2−β3.

FIG. 22C shows binding FACS of extracellular anti-Nav1.7α ISVDs on stable huNav1.7α-rhNav1.7α chimera cell line CHO FlpIn Nav1.7α(F276V)+β1−β2−β3.

FIG. 22D shows binding FACS of extracellular anti-Nav1.7α ISVDs on stable huNav1.7α-rhNav1.7α chimera cell line CHO FlpIn Nav1.7α(R277Q)+β1−β2−β3.

FIG. 22E shows binding FACS of extracellular anti-Nav1.7α ISVDs on stable huNav1.7α-rhNav1.7α chimera cell line CHO FlpIn Nav1.7α(E281V)+β1−β2−β3.

FIG. 22F shows binding FACS of extracellular anti-Nav1.7α ISVDs on stable huNav1.7α-rhNav1.7α chimera cell line CHO FlpIn Nav1.7α(V331M)+β1−β2−β3.

FIG. 22G shows a schematic representation of the extracellular polymorphisms between huNav1.7α and rhNav1.7α on an huNav1.7α model viewed from the extracellular side.

FIG. 23A shows a schematic illustrating the IonFlux 16 single pulse protocol.

FIG. 23B shows a schematic illustrating the IonFlux 16 two pulse protocol.

FIG. 24A shows an IonFlux 16 dose response titration of F0103265B04, F0103362B08, F0103387G04 and F0103387G05 using the single pulse (P1) protocol.

FIG. 24B shows an IonFlux 16 dose response titration of F0103265B04, F0103362B08, F0103387G04 and F0103387G05 using two pulse (P2) protocol.

FIG. 25A shows an IonFlux 16 single high concentration dose response for F0103265B04, F0103275B05, and F0103262CO2 in HEK huNav1.7α+β1 cells using single pulse (P1) and two pulse (P2) protocols.

FIG. 25B shows an IonFlux 16 single high concentration dose response for F0103265B04, F0103275B05, and F0103262CO2 in HEK huNav1.7α cells using single pulse (P1) and two pulse (P2) protocols.

FIG. 25C shows an IonFlux 16 single high concentration dose response for F0103265B04, F0103275B05, and F0103262CO2 in CHO FlpIn huNav1.7α+β1−β2−β3 cells using single pulse (P1) and two pulse (P2) protocols.

FIG. 25D shows an IonFlux 16 single high concentration dose response for F0103262B06, F0103265A11, and F0103265B04 in CHO FlpIn huNav1.7α+β1−β2−β3 cells using single pulse (P1) and two pulse (P2) protocols.

FIG. 25E shows an IonFlux 16 single high concentration dose response for F0103262B06, F0103265A11, and F0103265B04 in HEK FlpIn huNav1.7α+β1−β2−β3 cells using single pulse (P1) and two pulse (P2) protocols.

FIG. 26 shows the results of an IonFlux 16 washout experiment using F0103265B04.

FIG. 27 shows the results of an IonFlux 16 time course experiment using F0103265B04.

FIG. 28 shows a sequence analysis of F0103275B05 (SEQ ID NO: 50) and F010387G04 (SEQ ID NO: 52) compared to the human VH3-JH consensus sequence (SEQ ID NO: 57), VHH2 consensus sequence (SEQ ID NO: 58), and sequenced optimized F0103387G04 (F0103387G04 SO; SEQ ID NO:59).

FIG. 29 shows a sequence analysis of F0103387G05 (SEQ ID NO: 53) compared to the human VH3-JH consensus sequence (SEQ ID NO: 57), VHH2 consensus sequence (SEQ ID NO: 58), and sequenced optimized F0103387G05 (F0103387G05_SO; SEQ ID NO:60).

FIG. 30 shows the Tm of F0103387G05 variants in function of pH. Dotted lines mark variants with H37Y substitution (see Table 30).

FIG. 31 shows a sequence analysis of F0103464B09 (SEQ ID NO: 55) compared to the human VH3-JH consensus sequence (SEQ ID NO: 57), VHH2 consensus sequence (SEQ ID NO: 58), and sequenced optimized F01034647B09 (F01034647B09_SO; SEQ ID NO:61).

FIG. 32 shows a schematic diagram of huNav1.7α. VSD=voltage sensing domain; PM=pore module; D=domain; S=transmembrane segment.

FIG. 33 shows results of a binding FACS of anti-Navβ2 ISVD F0103240B04 on stable cell lines.

FIG. 34A shows results of a binding ELISA of the shown anti-Navβ ISVDs binding to Navβ1. F0103240B04 is a potent anti-Navβ2 binder control and IRR022 is a negative control comprising an irrelevant binder. F0103478E09 weakly binds Navβ1.

FIG. 34B shows results of a binding ELISA of the shown anti-Navβ ISVDs binding to 132. F0103240B04 is a potent anti-Navβ2 binder control and IRR0022 is a negative control comprising an irrelevant binder. F0103492E09, F0103500E03, and F0103505D08 weakly bind 132.

FIG. 34C shows results of a binding ELISA of the shown anti-Navβ ISVDs binding to Navβ3. F0103240B04 is a potent anti-Navβ2 binder control and IRR0202 is a negative control comprising an irrelevant binder. None of the ISVDs bind Navβ3.

FIG. 35A, FIG. 35B, FIG. 35C, and FIG. 35D together show results of binding FACS of the shown anti-Navβ subunit ISVDs (12.3 nM) on transiently transfected cells. Positive controls anti-Navβ1, anti-Navβ2, and anti-Navβ3 are rabbit polyclonal antibodies specific for human Navβ1, Navβ2, and Navβ3, respectively.

FIG. 36A shows results of binding FACS of anti-Navβ ISVD F0103478E09 on various stable cell lines.

FIG. 36B shows results of binding FACS of anti-Navβ ISVD F0103492E09 on various stable cell lines.

FIG. 36C shows results of binding FACS of anti-Navβ ISVD F0103500E03 on various stable cell lines.

FIG. 36D shows results of binding FACS of anti-Navβ ISVD F0103505D08 on various stable cell lines.

FIG. 36E shows results of binding FACS of anti-Navβ ISVD F0103495D09 on various stable cell lines.

FIG. 37A shows the results of a competition FACS of Nav1.7α-Navβ bispecific ISVDs on stable CHO cell lines expressing human Nav1.7α-Navβ1-Navβ2-Navβ3 (Nav1.7-β1-β2-β3).

FIG. 37B shows the results of a competition FACS of Nav1.7α-Navβ bispecific ISVDs on stable CHO cell lines expressing rhesus Nav1.7α-Navβ1-Navβ2-Navβ3 (Nav1.7-β1-β2-β3).

FIG. 38A shows the results of a competition FACS of Nav1.7α-Navβ bispecific ISVDs on stable HEK cell lines expressing human Nav1.7α (Nav1.7).

FIG. 38B shows the results of a competition FACS of Nav1.7α-Navβ bispecific ISVDs on stable HEK cell lines human expressing Nav1.7α-Navβ1 (Nav1.7-β1).

FIG. 38C shows the results of a competition FACS of Nav1.7α-Navβ bispecific ISVDs on stable HEK cell lines expressing human Nav1.7α-Navβ1-Navβ2-Navβ3 (Nav1.7-β1-β2-β3).

FIG. 39A shows binding FACS of Nav1.7 binder F0103262C02 on stable huNav1.x paralog HEK293T cell lines. MFI=median fluorescence intensity; a-FLAG is a detection moiety.

FIG. 39B shows binding FACS of Nav1.7 binder F0103265B04 on stable huNav1.x paralog HEK293T cell lines. MFI=median fluorescence intensity; a-FLAG is a detection moiety.

FIG. 39C shows binding FACS of Nav1.7 binder F0103275B05 on stable huNav1.x paralog HEK293T cell lines. MFI=median fluorescence intensity; a-FLAG is a detection moiety.

FIG. 39D shows binding FACS of Nav1.7 binder F0103464B09 on stable huNav1.x paralog HEK293T cell lines. MFI=median fluorescence intensity; a-FLAG is a detection moiety.

FIG. 39E shows binding FACS of Nav1.7 binder F0103387G05 on stable huNav1.x paralog HEK293T cell lines. MFI=median fluorescence intensity; a-FLAG is a detection moiety.

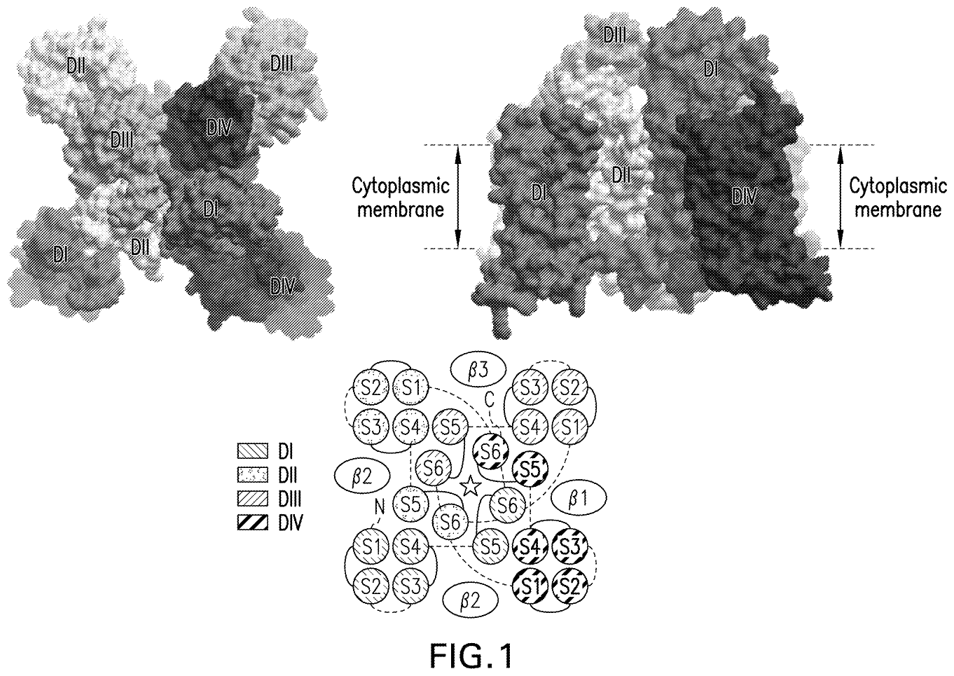

### DETAILED DESCRIPTION OF THE INVENTION

### Definitions

As used herein, the term “Nav1.7 binder” refers to an antibody, an antibody fragment, an immunoglobulin single variable domain (also referred to as “ISV” or ISVD″) or single domain antibody (also referred to as “sdAb”) that binds to Nav1.7α. An example of an ISVD is a Nanobody® molecule.

As used herein, the term “Navβ binder” refers to an antibody, an antibody fragment, an immunoglobulin single variable domain (also referred to as “ISV” or ISVD″) or single domain antibody (also referred to as “sdAb”) that binds to Navβ. The term “Navβ” comprises the terms “Navβ1” and “Navβ2”.

As used herein, “antibody” refers to an entire immunoglobulin, including recombinantly produced forms and includes any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, biparatopic antibodies, and chimeric antibodies. “Parental antibodies” are antibodies obtained by exposure of an immune system to an antigen prior to modification of the antibodies for an intended use, such as humanization of a non-human antibody for use as a human therapeutic antibody.

The term “antibody” refers, in one embodiment, to a conventional antibody, which is a protein tetramer comprising two heavy chains (HCs) and two light chains (LCs) inter-connected by disulfide bonds, or an antigen binding portion thereof, and in another embodiment, to a nonconventional antibody, which is a heavy chain antibody protein dimer comprising two heavy chains inter-connected by disulfide bonds and no light chains, or antigen binding portion thereof. In either embodiment, each heavy chain is comprised of a heavy chain variable region or domain (abbreviated herein as V_{H}) and a heavy chain constant region or domain. In certain naturally occurring IgG, IgD and IgA antibodies, the heavy chain constant region is comprised of three domains, C_{H}1, C_{H}2 and C_{H}3. In certain naturally occurring antibodies, each light chain is comprised of a light chain variable region or domain (abbreviated herein as V_{L}) and a light chain constant region or domain. The light chain constant region is comprised of one domain, CL. The human V_{H} includes six family members: V_{H}1, V_{H}2, V_{H}3, V_{H}4, V_{H}5, and V_{H}6 and the human V_{L} family includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes.

The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The CDRs form a binding domain that interacts with an antigen. The constant regions of the antibodies may mediate the binding of the immunoglobulin to host tissues or factors, including various cells of the immune system (e.g., effector cells) and the first component (C1q) of the classical complement system.

The constant domains or regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂, IgG₃, and IgG₄ constant domains in Beranger, et al., Ibid.

The variable domains or regions of the heavy and light chains contain a binding domain comprising the CDRs that interacts with an antigen. A number of methods are available in the art for defining or predicting the CDR amino acid sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence); Chothia numbering scheme is based on the location of the structural loop region (See
Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's
AbM antibody modelling software (see Karu et al, ILAR Journal 37: 132-141 (1995);
Contact numbering scheme is based on an analysis of the available complex crystal structures (See www.bioinf.org.uk: Prof Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).
   While there are several different methods for determining the amino acid sequences of the CDRs, the numbering of the entire variable region typically follows the Kabat numbering scheme with the particular CDR numbering scheme imposed thereupon.

The following general rules disclosed in www.bioinforg.uk: Prof. Andrew C. R. Martin's Group and reproduced in Table 1 below may be used to define or predict the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910⁻⁸⁹¹⁵ (1998); Xu et al., Immunity 13: 37-45 (2000).

A conventional antibody tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The heavy chain of a conventional antibody may or may not contain a terminal lysine (K), or a terminal glycine and lysine (GK).

As used herein, “antigen binding fragment” or “antigen binding portion” refers to fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; single-chain antibody molecules, e.g., sc-Fv; immunoglobulin single variable domain molecules, and multispecific antibodies formed from antibody fragments.

As used herein, the term “immunoglobulin single variable domain” (also referred to as “ISV” or ISVD″) or “single domain antibody (also referred to as “sdAb”) are terms that are used to refer to immunoglobulin variable domains (which may be heavy chain or light chain domains, including VH, VHH, or VL domains) that can form a functional antigen-binding site without interaction with another variable domain (e.g., without a VH/VL interaction as is required between the VH and VL domains of a conventional four-chain monoclonal antibody). The term “VH” refers to a heavy chain variable domain of a conventional antibody and the term “VHH” refers to the heavy chain variable domain of a non-conventional heavy chain antibody.

Examples of ISVDs include for example, VHHs, humanized VHHs, and/or a camelized VHs such as camelized human VHs), IgNAR domains, single domain antibodies such as dAbs™, which are VH domains or are derived from a VH domain or are VL domains or are derived from a VL domain. ISVDs that are based on and/or derived from heavy chain variable domains (such as VH or VHH domains) are generally preferred. Most preferably, an ISVD will be a VHH, a humanized VHH, or a camelized VH (such as a camelized human VH) or generally a sequence optimized VHH (e.g., optimized for chemical stability and/or solubility, maximum overlap with known human framework regions and maximum expression).

The term “Nanobody® molecule” is generally as defined in WO 2008/020079 or WO 2009/138519, and thus in a specific aspect denotes an VHH, a humanized VHH, or a camelized VH (such as a camelized human VH) or generally a sequence optimized VHH (such as, e.g., optimized for chemical stability and/or solubility, maximum overlap with known human framework regions and maximum expression). The term Nanobody® is a registered trademark of Ablynx N.V.

As used herein, “Nav1.7 binder” refers to a conventional antibody, heavy chain antibody, antigen binding fragment of an antibody or ISVD that binds to Nav1.7α. A Nav1.7 binder may be part of a larger molecule such as a multivalent, bispecific, or multispecific binder that includes one or more Nav1.7 binders and may include one or more binders to a target other than Nav1.7α (e.g., Navβ binder) and may comprises another functional element, such as, for example, a half-life extender (HLE), an Fc domain of an immunoglobulin, a targeting unit and/or a small molecule such a polyethylene glycol (PEG).

As used herein, “Navβ binder” refers to a conventional antibody, heavy chain antibody, antigen binding fragment of an antibody or ISVD that binds to Navβ1 or Navβ2. A Navβ binder may be part of a larger molecule such as a multivalent, bispecific, or multispecific binder that includes one or more Navβ binders and may include one or more binders to a target other than Navβ1 or Navβ2 (e.g., a Nav1.7 binder) and may comprise another functional element, such as, for example, a half-life extender (HLE), an Fc domain of an immunoglobulin, a targeting unit and/or a small molecule such as a PEG. Monovalent, monospecific and/or biparatopic Nav1.7 or Navβ binders are part of the present invention. A monovalent Nav1.7 or Navβ binder (e.g., ISVD such as a Nanobody® molecule) is a molecule that comprises a single antigen-binding domain. A bivalent or bispecific Nav1.7 binder (e.g., ISVD such as a Nanobody® molecule) comprises two antigen-binding domains, e.g., a Nav1.7-Navβ bispecific binder. A multivalent or multispecific Nav1.7 binder comprises more than one antigen-binding domain (e.g., 1, 2, 3, 4, 5, 6, or 7). When a multivalent or multispecific binder comprises only two antigen binding domains it may be referred to as a bispecific or bivalent binder.

For a general description of multivalent and multispecific polypeptides containing one or more ISVDs and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 1996/34103, WO 1999/23221, WO 2004/041862, WO 2006/122786, WO 2008/020079, WO 2008/142164 or WO 2009/068627.

As used herein, a “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

As used herein, a “Fab′ fragment” contains one light chain and a portion or fragment of one heavy chain that contains the VH domain and the C_{H}1 domain and also the region between the C_{H}1 and C_{H}2 domains, such that an interchain disulfide bond can be formed between the two heavy chains of two Fab′ fragments to form a F(ab′)₂ molecule.

As used herein, a “F(ab′)₂ fragment” contains two light chains and two heavy chains containing the V_{H} domain and a portion of the constant region between the C_{H}1 and C_{H}2 domains, such that an interchain disulfide bond is formed between the two heavy chains. An F(ab′)₂ fragment thus is composed of two Fab′ fragments that are held together by a disulfide bond between the two heavy chains. An “F(ab′)₂ fragment” can be the product of pepsin cleavage of an antibody.

As used herein, an “Fv region” comprises the variable regions from both the heavy and light chains but lacks the constant regions.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, an “Fc domain” or “Fc region” each refer to the fragment crystallizable region of an antibody. The Fc domain comprises two heavy chain fragments comprising the C_{H}1 and C_{H}2 domains of an antibody. The two heavy chain fragments are held together by two or more disulfide bonds and by hydrophobic interactions of the C_{H}3 domains. The Fc domain may be fused at the N-terminus or the C-terminus to a heterologous protein.

As used herein, a “diabody” refers to a small antibody fragment with two antigen-binding regions, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementarity domains of another chain and create two antigen-binding regions. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, “isolated” antibodies or antigen-binding fragments thereof (e.g., Nav1.7 and Navβ binders) are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, a “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991)J Mol. Biol. 222: 581-597, for example. See also Presta (2005) J. Allergy Clin. Immunol. 116:731.

As used herein, a “humanized ISVD” or “humanized antibody” refers to forms of Nav1.7 binders that contain sequences from both human and non-human (e.g., llama, murine, rat) antibodies. In general, the humanized Nav1.7 and Navβ binders will comprise all of at least one, and typically two, variable domains, in which the hypervariable loops correspond to those of a non-human immunoglobulin, and all or substantially all of the framework (FR) regions are those of a human immunoglobulin sequence. The humanized Nav1.7 and/or Navβ binder may optionally comprise at least a portion of a human immunoglobulin constant region (Fc).

“Humanization” (also called Reshaping or CDR-grafting) is now a well-established technique for reducing the immunogenicity of monoclonal antibodies (mAbs) from xenogeneic sources (commonly rodent or camelids) and for improving the effector functions (ADCC, complement activation, C1q binding). The engineered mAb is engineered using the techniques of molecular biology, however simple CDR-grafting of the rodent complementarity-determining regions (CDRs) into human frameworks often results in loss of binding affinity and/or specificity of the original mAb. In order to humanize an antibody, the design of the humanized antibody includes variations such as conservative amino acid substitutions in residues of the CDRs, and back substitution of residues from the rodent mAb into the human framework regions (backmutations). The positions can be discerned or identified by sequence comparison for structural analysis or by analysis of a homology model of the variable regions' 3D structure. The process of affinity maturation has most recently used phage libraries to vary the amino acids at chosen positions. Similarly, many approaches have been used to choose the most appropriate human frameworks in which to graft the rodent CDRs. As the datasets of known parameters for antibody structures increases, so does the sophistication and refinement of these techniques. Consensus or germline sequences from a single antibody or fragments of the framework sequences within each light or heavy chain variable region from several different human mAbs can be used. Another approach to humanization is to modify only surface residues of the rodent sequence with the most common residues found in human mAbs and has been termed “resurfacing” or “veneering.” Known human Ig sequences are disclosed, e.g.,
www.ncbi.nlm.nih.gov/entrez/query.fcgi; www.ncbi.nih.gov/igblast;
www.atcc.org/phage/hdb.html; www.kabatdatabase.com/top.html;
www.antibodyresource.com/onlinecomp.html; www.appliedbiosystems.com;
www.biodesign.com; antibody.bath.ac.uk; www.unizh.ch; www.cryst.bbk.ac.uk/.about.ubcgO7s; Kabat et al., Sequences of Proteins of Immunological Interest, U.S. Dept. Health (1983), each entirely incorporated herein by reference. Often, the human or humanized antibody is substantially non-immunogenic in humans.

As used herein, “non-human amino acid sequences” with respect to antibodies or immunoglobulins refers to an amino acid sequence that is characteristic of the amino acid sequence of a non-human mammal. The term does not include amino acid sequences of antibodies or immunoglobulins obtained from a fully human antibody library where diversity in the library is generated in silico (See for example, U.S. Pat. No. 8,877,688 or 8,691,730).

As used herein, “effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

As used herein, “conservatively modified variants” or “conservative substitution” refers to substitutions of amino acids with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in the table below.

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

As used herein, the term “epitope” or “antigenic determinant” refers to a site on an antigen (e.g., Nav1.7α, Navβ1, Navβ2) to which a binder specifically binds. Epitopes within protein antigens can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. A contiguous linear epitope comprises a peptide domain on an antigen comprising at least 3, 4, 5, 6, 7, 8, 9, 10, 11, 12, 13, 14 or 15 amino acids. A noncontiguous conformational epitope comprises one or more peptide domains or regions on antigen bound by a binder interspersed by one or more amino acids or peptide domains not bound by the binder, each domain independently comprises at least 3, 4, 5, 6, 7, 8, 9, 10, 11, 12, 13, 14 or 15 amino acids. Methods for determining what epitopes are bound by a given binder (i.e., epitope mapping) are well known in the art and include, for example, immunoblotting and immunoprecipitation assays, wherein overlapping or contiguous peptides (e.g., from Nav1.7α, Navβ1, Navβ2) are tested for reactivity with a given binder. Methods of determining spatial conformation of epitopes include techniques in the art and those described herein, for example, x-ray crystallography, 2-dimensional nuclear magnetic resonance, and HDX-MS (see, e.g., Epitope Mapping Protocols in Methods in Molecular Biology, Vol. 66, G. E. Morris, Ed. (1996)).

The term “epitope mapping” refers to the process of identification of the molecular determinants on the antigen involved in antibody-antigen recognition.

The term “binds to the same epitope” with reference to two or more binders means that the binders bind to the same segment of amino acid residues on a target, as determined by a given method. Techniques for determining whether a particular binder binds to the “same epitope” as the Nav1.7 or Navβ binders described herein include, for example, epitope mapping methods, such as, x-ray analyses of crystals of Nav1.7α:Nav1.7 binder or Navβ:Navβ binder complexes, which provides atomic resolution of the epitope, and hydrogen/deuterium exchange mass spectrometry (HDX-MS). Other methods that monitor the binding of the antibody to antigen fragments (e.g. proteolytic fragments) or to mutated variations of the antigen where loss of binding due to a modification of an amino acid residue within the antigen sequence is often considered an indication of an epitope component (e.g. alanine scanning mutagenesis—Cunningham & Wells (1985) Science 244:1081). In addition, computational combinatorial methods for epitope mapping can also be used. These methods rely on the ability of the binder of interest to affinity isolate specific short peptides from combinatorial phage display peptide libraries.

Binders that “compete with a binder of the present invention for binding to a target antigen” refer to binders that inhibit (partially or completely) the binding of the Nav1.7 binder of the present invention to Nav1.7α or Navβ binder to Navβ. Whether two binders compete with each other for binding to the target antigen, i.e., whether and to what extent one binder inhibits the binding of the other binder to the target antigen, may be determined using known competition experiments. In certain embodiments, a binder competes with, and inhibits binding of a binder of the present invention to the target antigen by at least 10%, 20%, 30%, 40%, 50%, 60%, 70%, 80%, 90% or 100%. The level of inhibition or competition may be different depending on which binder is the “blocking binder” (i.e., the unlabeled binder that is incubated first with the target antigen). Competition assays can be conducted as described, for example, in Ed Harlow and David Lane, Cold Spring Harb Protoc; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing Nav1.7 binders bind to the same epitope as defined herein.

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, “specifically binds” refers, with respect to a target antigen, to the preferential association of a binder, in whole or part, with the target antigen and not to other molecules, particularly molecules found in human blood or serum. Binders as shown herein typically bind specifically to the target antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, a binder that “specifically binds” or “binds specifically” to a target antigen refers to a binder that binds to the target antigen with high affinity, which means having a K_{D} of 10⁻⁷ M or less, in particular embodiments a K_{D} of 10⁻⁸ M or less, or 5×10⁻⁹ M or less, or between 10⁻⁸ M and 10⁻¹¹ M or less, but does not bind with measurable binding to closely related proteins such as human Nav1.1α, human Nav1.2α, human Nav1.3a, humanNav.1.4α, human Nav1.5α, human Nav 1.6α, or human Nav1.8α as determined in a cell ELISA or Surface Plasmon Resonance assay (SPR; Biacore) using 10 μg/mL antibody.

As used herein, an antigen is “substantially identical” to a given antigen if it exhibits a high degree of amino acid sequence identity to the given antigen, for example, if it exhibits at least 80%, at least 90%, at least 95%, at least 97%, or at least 99% or greater amino acid sequence identity to the amino acid sequence of the given antigen. By way of example, an antibody that binds specifically to human Nav1.7α or Navβ may also cross-react with Nav1.7α or Navβ from certain non-human primate species (e.g., rhesus monkey or cynomolgus monkey). The term specifically excludes human Nav1.1α, human Nav1.2α, human Nav1.3a, humanNav.1.4α, human Nav1.5α, human Nav 1.6α, and human Nav1.8a.

As used herein, “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

As used herein, “treat” or “treating” means to administer a therapeutic agent, such as a composition containing any of the Nav1.7 and/or Navβ binders of the present invention, topically, subcutaneously, intramuscular, intradermally, or systemically to an individual experiencing chronic pain. The amount of a therapeutic agent that is effective to alleviate chronic pain in the individual may vary according to factors such as the injury or disease state, age, and/or weight of the individual, and the ability of the therapeutic agent to elicit a desired response in the individual. Whether chronic pain has been alleviated can be assessed by the individual and/or any clinical measurement typically used by physicians or other skilled healthcare providers to assess the severity or progression status of chronic pain. Thus, the terms denote that a beneficial result has been or will be conferred on a human or animal individual experiencing chronic pain.

As used herein, “treatment,” as it applies to a human or veterinary individual, refers to therapeutic treatment, as well as diagnostic applications. “Treatment” as it applies to a human or veterinary individual, encompasses contact of the antibodies or antigen binding fragments of the present invention to a human or animal subject.

As used herein, “therapeutically effective amount” refers to a quantity of a specific substance sufficient to achieve a desired effect in an individual being treated. For instance, this may be the amount necessary to inhibit or reduce the severity of chronic pain in an individual.

As used herein, the term “effector-silent” as used herein refers to an antibody, antibody fragment, HC constant domain, or Fc domain thereof that displays (i) no measurable binding to one or more Fc receptors (FcRs) as may be measured in a surface plasmon resonance (SPR) assay (e.g., Biacore™ assay) wherein an association constant in the micromolar range indicates no measurable binding or (ii) measurable binding to one or more FcRs as may be measured in SPR assay that is reduced compared to the binding that is typical for an antibody, antibody fragment, HC constant domain or Fc domain thereof the same isotype. In particular embodiments, the antibody, antibody fragment, HC constant domain, or Fc domain thereof may comprise one or more mutations in the HC constant domain and the Fc domain in particular such that the mutated an antibody, antibody fragment, HC constant domain or Fc domain thereof has reduced or no measurable binding to FcγRIIIa, FcγRIIa, and FcγRI compared to a wild-type antibody of the same isotype as the mutated antibody. In particular embodiments, the affinity or association constant of an effector-silent an antibody, antibody fragment, HC constant domain or Fc domain thereof to one or more of FcγRIIIa, FcγRIIa, and FcγRI is reduced by at least 1000-fold compared to the affinity of the wild-type isotype; reduced by at least 100-fold to 1000-fold compared to the affinity of the wild-type isotype reduced by at least 50-fold to 100-fold compared to the affinity of the wild-type isotype; or at least 10-fold to 50-fold compared to the affinity of the wild-type isotype. In particular embodiments, the effector-silent an antibody, antibody fragment, HC constant domain, or Fc domain thereof has no detectable or measurable binding to one or more of the FcγRIIIa, FcγRIIa, and FcγRI as compared to binding by the wild-type isotype. In general, effector-silent an antibody, antibody fragment, HC constant domain, or Fc domain thereof will lack measurable antibody-dependent cell-mediated cytotoxicity (ADCC) activity. An ISVD not fused or linked to an effector-silent HC constant domain or Fc domain thereof displays no detectable or measurable binding to one or more of FcγRIIIa, FcγRIIa, or FcγRI. SPR assays measure binding of an effector-silent antibody, antibody fragment, HC constant domain or Fc domain thereof, against human FcRs.

### INTRODUCTION

Patients with loss of function mutations in the gene encoding the Nav1.7α channel (SCN9A) show profound insensitivity to pain from birth on. In contrast, gain of function mutations can result in chronic pain disorders. Nav1.7α channels predominantly expressed in peripheral C-fiber nociceptors are therefore a drug target of great interest for treatment of various pain conditions. We have identified ISVDs (Nav1.7 binders) that inhibit Nav1.7α channels with exquisite selectivity over other Nav channel paralogs. Functional inhibitory Nav1.7 activity of the Nav1.7 binders was assessed in automated in vitro patch clamp assays. IC₅₀ values in the nanomolar range have been measured. In vivo target modulation in the tissue of interest (peripheral C-fiber nociceptors) was demonstrated in Rhesus microneurography assays. The potential advantages of injectable Nav1.7 binders for the treatment of chronic pain syndromes, such as painful diabetic peripheral neuropathy and osteoarthritis pain, are specificity and extended half-life. Clinical differentiation will be based on improved or comparable efficacy with better side effect profile versus standard of care.

In an embodiment of the invention, any Nav1.7 binder or other binder as set forth herein comprises, where applicable, a substitution of the amino acid at position 11 to the amino acid V and a substitution of the amino acid at position 89 to the amino acid L. In further embodiments, the Nav1.7 binder further includes a substitution of the amino acid at position 110 to the amino acid T, K, or Q. In further embodiments, the amino acid at position 112 is substituted with the amino acid S, K or Q. In each case wherein the numbering is according to the Kabat numbering scheme.

### Nav1.7 Sodium Ion Channel

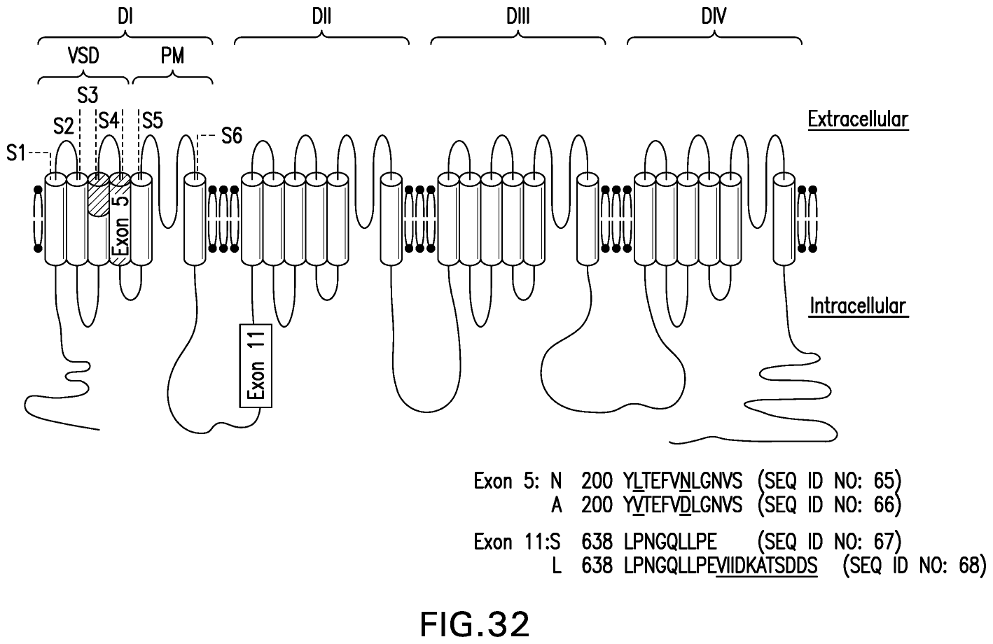

The α-subunits of the Nav1.7 channel are polypeptide chains of 1977 amino acids that are folded into four homologous (but not identical) domains termed DI-DIV that are linked by three intracellular loops (L1-L3). Each domain has six transmembrane segments (S1-S6) with S1-S4 in each domain comprising a voltage sensing domain (VSD), and S5-S6 together with their extracellular linker (including the P-loop) included in the pore domain (PD) (Catterall (2000) Neuron 26:13-25; Guy & Seetharamulu (1986) Proceedings of the National Academy of Sciences of the United States of America 83: 508-512; Noda et al. (1984) Nature 312:121-127). Thus, each α-subunit has four distinct VSDs and four PDs which assemble to form one sodium-selective pore. Sodium is selectivity achieved in the extracellular portion of the pore domain by tight association of the four P-loops that re-enter the membrane between the S5 and S6 segments in DI-DIV and includes several negatively charged residues (aspartic acid and glutamic acid) (Catterall 2000). The human Nav1.7α comprises the amino acid sequence set forth in SEQ ID NO: 1. Domain I of the human Nav1.7α consists of the amino acid sequence shown in SEQ ID NO: 63 and the Domain I S5-S6 loop is shown in SEQ ID NO: 64. The amino acid sequence for the rhesus monkey NAV1.7α is shown in SEQ ID NO: 2, which has 99% identity with the human Nav1.7α. A schematic representation of Nav1.7α is shown in FIG. 32.

### Nav1.7 Binders

The present invention provides Nav1.7 binders (e.g., ISVDs) that bind to Nav1.7α and methods of use of the binders for or in the treatment or prevention of disease. In an embodiment of the Nav1.7 binders, the Nav1.7 binders are antagonistic anti-NaV1.7α ISVDs. In further embodiments, the Nav1.7 binder antagonizes the activity of the Nav1.7 channel, for example, by blocking the channel, which may be by physically blocking or closing the Nav1.7 pore to Na⁺ flux or by conformationally changing the Nav1.7 channel to an inactive state.

The Nav1.7 binders include binders that bind to the Domain I S5-S6 loop of the human Nav1.7α comprising amino acids 276 through 331 thereof (e.g., FRNSLENNETLESIMNTLESEEDFRKYFYYLEGSKDALLCGFSTDSGQCPEGYTCV (SEQ ID NO: 62)), and heteromeric channels in which the Nav1.7α is complexed with one or more beta subunits such as β1, β2,β3, and/or β4. In an embodiment of the invention, the Nav1.7 binder contacts one or more of the following Nav1.7α amino acid residues: F276, R277, E281, and V331 as shown underlined in the amino acid sequence above. In a further embodiment, the Nav1.7 binder contacts the following four Nav1.7α amino acid residues: F276, R277, E281, and V331. Thus, in particular embodiments, the Nav1.7 binders of the present invention bind to an epitope on Nav1.7α comprising amino acid residues F276, R277, E281, and V331. In a further embodiment, the epitope consists of amino acid residues F276, R277, E281, and V331.

In particular embodiments of the invention, the Nav1.7 binder binds to Nav1.7α having one or more mutations at residue F276, R277, E281, and/or V331 with lower affinity than to human Nav1.7α lacking such mutations. In particular embodiments of the invention, the binder binds to human Nav1.7α comprising one or more mutations at positions Q1530, H1531, and E1534 with a substantially similar affinity to that of human Nav1.7α lacking said mutations. In particular embodiments of the invention, the binder binds to human Nav1.7α comprising mutations at positions Q1530, H1531, and E1534 with a substantially similar affinity to that of human Nav1.7α lacking said mutations. In further embodiments of the invention, the Nav1.7 binder does not bind to rhesus monkey Nav1.7α or binds with a lower affinity than to human Nav1.7α.

In an embodiment of the invention, the Nav1.7 binder binds to human Nav1.7α with substantially similar affinity to human Nav1.7α lacking one more of loops other than the domain 1 S5-S6 loop.

The Nav1.7 binders of the present invention comprise three complementarity determining regions (CDRs) having amino acid sequences selected from the tables below. The CDR amino acid sequences shown in Table 2 and Table 3 are set forth according to the AbM numbering scheme for defining CDR amino acid sequences. A particular CDR amino acid sequence defined by any one of the other schemes advanced for defining CDR amino acid sequences (See Table 1) may have more or less amino acids than shown for CDR amino acid sequences identified according to the AbM numbering scheme but will overlap the CDR amino acid sequences defined according the AbM numbering scheme. Thus, the CDR amino acid sequences shown herein are not to be construed as limiting and any Nav1.7 binder in which the CDR amino acid sequences have been defined by any other numbering scheme will fall within the scope of the Nav1.7 binders of the present invention provided the amino acid sequences for such Nav1.7 binders comprise the amino acid sequences defined for the three CDR amino acid sequences as shown in Table 2 and Table 3. Thus, regardless of the method used to define the CDRs of a Nav1.7 binder (e.g., Kabat, AbM, Clothia, IMGT, Contact, etc.), any Nav1.7 binder that comprises the three amino acid sequences defined for CDR1, CDR2, and CDR3 for any of the Nav1.7 binders shown in Table 2 and Table 3 are Nav1.7 binders of the present invention.

The Nav1.7 binders comprise three CDRs and four Frameworks (FR) in the following alignment FR1-CDR1-FR2-CDR2-FR3-CDR3-FR4. The Nav1.7 binder CDRs may comprise CDRs comprising the following amino acid sequences.

TABLE 2

| Nav1.7 binder | CDR1 | CDR2 | CDR3 |
|---|---|---|---|
| F0103262B06 | TRTFSTYAMG | HINFSGSSTRY | ARWVAGPPRYDYEY |
| | (SEQ ID NO: 247) | (SEQ ID NO: 248) | (SEQ ID NO: 249) |
| F0103262C02 | GLPFGLYILG | AISRSGRDTV | DSVPRGTPTITESEYAI |
| | (SEQ ID NO: 250) | (SEQ ID NO: 251) | (SEQ ID NO: 252) |
| F0103265A11 | GMLFNANTQG | FIFSGGYTN | SRY |
| | (SEQ ID NO: 253) | (SEQ ID NO: 254) | |
| F0103265B04 | SFIFSNNYME | RITGRGNTN | LWYGGRA |
| | (SEQ ID NO: 256) | (SEQ ID NO: 257) | (SEQ ID NO: 258) |
| F0103362B08 | VRPFSTSAMG | GILWNGIVTY | DRDYGGRSFSAYEYEY |
| | (SEQ ID NO: 259) | (SEQ ID NO: 260) | (SEQ ID NO: 261) |
| F0103454D07 | GGIININYIA | RISSDDTIK | LITPWTGDTRTY |
| | (SEQ ID NO: 262) | (SEQ ID NO: 263) | (SEQ ID NO: 264) |
| F0103275B05 | GSIFNINSMA | SSTNGGSTN | LLQPSIYDISRTY |
| | (SEQ ID NO: 196) | (SEQ ID NO: 198) | (SEQ ID NO: 200) |
| F0103387G05 | GRILRIGYMR | RITDDSATD | LVTASVRGGSIHSGTY |
| | (SEQ ID NO: 201) | (SEQ ID NO: 202) | (SEQ ID NO: 206) |
| F0103464B09 | SRAFIRDVFTG | RIYNGGNTN | SGTINTGREYRSGDY |
| | (SEQ ID NO: 207) | (SEQ ID NO: 213) | (SEQ ID NO: 219) |
| F0103387G04 | GPVFNINKMA | SVTPTGSIS | LLQPDSYSNTRTY |
| | (SEQ ID NO: 221) | (SEQ ID NO: 223) | (SEQ ID NO: 225) |

In particular embodiments of the invention, the Nav1.7 binder comprises a CDR1 comprising the amino acid sequence set forth in SEQ ID NO: 247, a CDR2 comprising the amino acid sequence set forth in SEQ ID NO: 248, and a CDR3 comprising the amino acid sequence set forth in SEQ ID NO: 249.

In particular embodiments of the invention, the Nav1.7 binder comprises a CDR1 comprising the amino acid sequence set forth in SEQ ID NO: 250, a CDR2 comprising the amino acid sequence set forth in SEQ ID NO: 251, and a CDR3 comprising the amino acid sequence set forth in SEQ ID NO: 252.

In particular embodiments of the invention, the Nav1.7 binder comprises a CDR1 comprising the amino acid sequence set forth in SEQ ID NO: 253, a CDR2 comprising the amino acid sequence set forth in SEQ ID NO: 254, and a CDR3 comprising the amino acid sequence SRY.

In particular embodiments of the invention, the Nav1.7 binder comprises a CDR1 comprising the amino acid sequence set forth in SEQ ID NO: 256, a CDR2 comprising the amino acid sequence set forth in SEQ ID NO: 257, and a CDR3 comprising the amino acid sequence set forth in SEQ ID NO: 258.

In particular embodiments of the invention, the Nav1.7 binder comprises a CDR1 comprising the amino acid sequence set forth in SEQ ID NO: 259, a CDR2 comprising the amino acid sequence set forth in SEQ ID NO: 260, and a CDR3 comprising the amino acid sequence set forth in SEQ ID NO: 261.

In particular embodiments of the invention, the Nav1.7 binder comprises a CDR1 comprising the amino acid sequence set forth in SEQ ID NO: 262, a CDR2 comprising the amino acid sequence set forth in SEQ ID NO: 263, and a CDR3 comprising the amino acid sequence set forth in SEQ ID NO: 264.

In particular embodiments of the invention, the Nav1.7 binder comprises a CDR1 comprising the amino acid sequence set forth in SEQ ID NO: 196, a CDR2 comprising the amino acid sequence set forth in SEQ ID NO: 198, and a CDR3 comprising the amino acid sequence set forth in SEQ ID NO: 200.

In particular embodiments of the invention, the Nav1.7 binder comprises a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 206.

In particular embodiments of the invention, the Nav1.7 binder comprises a CDR1 comprising the amino acid sequence set forth in SEQ ID NO: 207, a CDR2 comprising the amino acid sequence set forth in SEQ ID NO: 213, and a CDR3 comprising the amino acid sequence set forth in SEQ ID NO: 219.

In particular embodiments of the invention, the Nav1.7 binder comprises a CDR1 comprising the amino acid sequence set forth in SEQ ID NO: 221, a CDR2 comprising the amino acid sequence set forth in SEQ ID NO: 223, and a CDR3 comprising the amino acid sequence set forth in SEQ ID NO: 225.

In a further embodiments of the invention, the Nav1.7 binder comprises three CDRs having an amino acid sequence as set forth in Table 3.

TABLE 3
F0103275B05 Family

| SEQ | | SEQ | | SEQ | |
|---|---|---|---|---|---|
| ID | | ID | | ID | |
| NO: | CDR1 | NO: | CDR2 | NO: | CDR3 |
| 196 | GSIFNINSMA | 198 | SSTNGGSTN | 200 | LLQPSIYDISRTY |
| 197 | GSIFNINRMA | 199 | YSTNGGDTN | | |

F0103387G05 Family

| SEQ | | SEQ | | SEQ | |
|---|---|---|---|---|---|
| ID | | ID | | ID | |
| NO: | CDR1 | NO: | CDR2 | NO: | CDR3 |
| 201 | GRILRIGYMR | 202 | RITDDSATD | 206 | LVTASVRGGSIHSGTY |
| | | 203 | RITGGSATG | | |
| | | 204 | RITDDSATG | | |
| | | 205 | RITGGSATG | | |

F0103464B09 Family

| SEQ | | SEQ | | SEQ | |
|---|---|---|---|---|---|
| ID | | ID | | ID | |
| NO: | CDR1 | NO: | CDR2 | NO: | CDR3 |
| 207 | SRAFIRDVFTG | 213 | RIYNGGNTN | 219 | SGTINTGREYRSGDY |
| 208 | SRAFIRDLFTG | 214 | RIYNEGNTN | | |
| 209 | SRQFIRDVFTG | 215 | RIYNEGNTQ | | |
| 210 | HRQFIRDVFTG | 216 | RIYESGNTQ | | |
| 211 | HRAFIRDVFTG | 217 | RIYESGNTN | | |
| 212 | HRAFIRDLFTG | 218 | RIYNEGNTN | | |

F0103387G04 Family

| SEQ | | SEQ | | SEQ | |
|---|---|---|---|---|---|
| ID | | ID | | ID | |
| NO: | CDR1 | NO: | CDR2 | NO: | CDR3 |
| 221 | GPVFNINKMA | 223 | SVTPTGSIS | 225 | LLQPDSYSNTRTY |
| 222 | GPVFNINRMA | 224 | YVTPTGDIS | 226 | LLQPRRYSNTRTY |
| | | | | 227 | LLQPDSYSITRTY |
| | | | | 228 | LLQPRSYSITRTY |
| | | | | 229 | LLQPRSYSNTRTY |
| | | | | 230 | LLQPSSYSITRTY |
| | | | | 231 | LLQPNVYSITRTY |
| | | | | 232 | LLQPDVYSITRTY |
| | | | | 233 | LLQPSSYSGTRTY |

Amino acid residues in bold face mark those amino acids that are different from the amino acid in the parental sequence.

In particular embodiments of the invention, the Nav1.7 binder comprises a CDR1 comprising the amino acid sequence set forth in SEQ ID NO: 196 or SEQ ID NO: 197; a CDR2 comprising the amino acid sequence set forth in SEQ ID NO: 198 or SEQ ID NO: 199; and, a CDR3 comprising the amino acid sequence set forth in SEQ ID NO: 200.

In particular embodiments of the invention, the Nav1.7 binder comprises a CDR1 comprising the amino acid sequence set forth in SEQ ID NO: 201; a CDR2 comprising the amino acid sequence set forth in SEQ ID NO: 202, SEQ ID NO: 203, SEQ ID NO: 204, or SEQ ID NO: 205; and, a CDR3 comprising the amino acid sequence set forth in SEQ ID NO: 206.

In particular embodiments of the invention, the Nav1.7 binder comprises a CDR1 comprising the amino acid sequence set forth in SEQ ID NO: 207, SEQ ID NO: 208, SEQ ID NO: 209, SEQ ID NO: 210, SEQ ID NO: 211, or SEQ ID NO: 212; a CDR2 comprising the amino acid sequence set forth in SEQ ID NO: 213, SEQ ID NO: 214, SEQ ID NO: 215, SEQ ID NO: 216, SEQ ID NO: 217, or SEQ ID NO: 218; and a CDR3 comprising the amino acid sequence set forth in SEQ ID NO: 219.

In particular embodiments of the invention, the Nav1.7 binder comprises a CDR1 comprising the amino acid sequence set forth in SEQ ID NO: 201 or SEQ ID NO: 222; a CDR2 comprising the amino acid sequence set forth in SEQ ID NO: 223 or SEQ ID NO: 224; and a CDR3 comprising the amino acid sequence set forth in SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, or SEQ ID NO: 233.

In a further embodiment of the invention, the Nav1.7 binder comprises a CDR1 comprising the amino acid sequence set forth in SEQ ID NO: 201; a CDR2 comprising the amino acid sequence set forth in SEQ ID NO: 205; and, a CDR3 comprising the amino acid sequence set forth in SEQ ID NO: 206.

In particular embodiments of the invention, the Nav1.7 binder comprises a CDR1 comprising the amino acid sequence set forth in SEQ ID NO: 211; a CDR2 comprising the amino acid sequence set forth in SEQ ID NO: 215; and, a CDR3 comprising the amino acid sequence set forth in SEQ ID NO: 219.

In particular embodiments of the invention, the Nav1.7 binder comprises a CDR1 comprising the amino acid sequence set forth in SEQ ID NO: 222; a CDR2 comprising the amino acid sequence set forth in SEQ ID NO: 223; and, a CDR3 comprising the amino acid sequence set forth in SEQ ID NO: 233.

As recited above, the Nav1.7 binders comprise four frameworks: FR1, FR2, FR3, and FR4 wherein the Nav1.7 binder is a single polypeptide having the structure beginning from the N-terminus FR1-CDR1-FR2-CDR2-FR3-CDR3-FR4. The numbering of the frameworks may be as shown herein be according to the Kabat numbering scheme and the junction between each framework and a CDR may be defined according to the AbM numbering scheme as shown herein. In particular embodiments, the Nav1.7 binders comprise the VHH2-consensus frameworks FR1, FR2, FR3, and FR4, wherein FR1 has the amino acid sequence set forth in SEQ ID NO: 268, FR2 has the amino acid sequence set forth in SEQ ID NO: 269, FR3 has the amino acid sequence set forth in SEQ ID NO: 270, and FR4 has the amino acid sequence set forth in SEQ ID NO: 271. In further embodiments, each framework may comprise one or more substitutions and or insertions with the proviso that the Nav1.7 binder is capable of binding human Nav1.7α. In further embodiments, frameworks may comprise one or more of the substitutions and/or insertions shown in Table 4 in any combination. In further embodiments, FR1 may comprise one or more of the substitutions shown for FR1 in Table 4. In further embodiments, FR2 may comprise one or more of the substitutions shown for FR2 in Table 4. In further embodiments, FR3 may comprise one or more of the substitutions shown for FR3 in Table 4. In further embodiments, FR4 may comprise one of the substitutions shown for FR4 in Table 4. In a further embodiment, each framework comprises at least one amino acid substitution. In a further embodiment, the Nav1.7 binder comprises at least one substitution and/or insertion shown in Table 4 for each of FR1, FR2, FR3, and FR4. In a further embodiment, the Nav1.7 binder comprises the one substitution or specific substitution and/or insertion combination shown in Table 4 for each of FR1, FR2, FR3, and FR4.

In particular embodiments, the ISVD framework comprises one or more substitutions to minimize binding to pre-existing antibodies. Pre-existing antibodies are antibodies existing in the body of a patient prior to receipt of an ISVD and are immunoglobulins mainly of the IgG class that are present in varying degrees in up to 50% of the human population and that bind to critical residues clustered at the C-terminal region of ISVDs. The ISVDs of the present invention are based, in part, in llama antibodies whose C-terminal constant domains have been removed; thus, exposing the neo-epitopes in the C-terminus of the resulting VHH to pre-existing antibody binding. It has been discovered that the combination of mutations of residues 11 and 89 (e.g., L11V and I89L or V89L) led to a surprising lack of pre-existing antibody binding. Mutations in residue 112 have also been shown to remarkably reduce pre-existing antibody binding. Buyse & Boutton (WO2015/173325) included data showing that the combination of an L11V and V89L mutation provided a remarkable improvement in reducing pre-existing antibody binding compared to an L11V mutation alone or a V89L mutation alone. For example, Table H of Buyse & Boutton on page 97 showed comparative data for an ISVD with a V89L mutation alone (with or without C-terminal extension) and the same ISVD with a V89L mutation in combination with an L11V mutation (again, with or without a C-terminal extension). Also, although generated in two separate experiments, the data shown in Table H for the L11V/V89L combination as compared to the data given in Table B for an L11V mutation alone (in the same ISVD) showed that the pre-existing antibody binding reduction that is obtained by the L11V/V89L combination was greater than that for the L11V mutation alone. Since the llama antibody scaffold structure is known to be very highly conserved, the effect of the mutations at positions 11 and 89 is very likely to exist for any ISVD. Thus, in embodiments herein, the ISVD comprises at least the L11V/V89L substitutions in the framework regions.

In a further embodiment, FR1 comprises at least an L11V substitution and FR3 comprises at least a V89L substitution. In a further still embodiment, the Nav1.7 binder may comprise one of the 125 specific sets of FR1, FR2, FR3, and FR4 combinations shown in Table 4. In any one of the above embodiments, the FR1 may further comprise a Q1E or a Q1D amino acid substitution.

TABLE 4

| # | FR1 | FR2 | FR3 | FR4 |
|---|---|---|---|---|
| 1 | NC | NC | N93R | NC |
| 2 | L11V | R39Q | T83R, V89L | NC |
| 3 | L11V | NC | R76N, T83R, V89L | NC |
| 4 | L11V | NC | T83R, V89L | NC |
| 5 | L11V | R39Q | R76N, T83R, V89L | NC |
| 6 | L11V | R39Q | R76_V78insT, T83R, V89L | NC |
| 7 | L11V | NC | R76_V78insT, T83R, V89L | NC |
| 8 | L11V | NC | R76_V78insT, R76N, | NC |
| | | | T83R, V89L | |
| 9 | L11V | R39Q | R76_V78insT, R76N, | NC |
| | | | T83R, V89L | |
| 10 | L11V | NC | R76N, T83R, V89L, N93R | NC |
| 11 | L11V | NC | T83R, V89L, N93R | NC |
| 12 | L11V | R39Q | T83R, V89L, N93R | NC |
| 13 | L11V | R39Q | R76N, T83R, V89L, N93R | NC |
| 14 | D23A | NC | NC | NC |
| 15 | D23A | NC | NC | NC |
| 16 | L11V, A14P, | G40A, A41P | N82bS, N83R, V89L | R105Q |
| | D23A | | | |
| 17 | L11V, A14P, | H37Y, G40A, | N82bS, N83R, V89L | R105Q |
| | D23A | A41P | | |
| 18 | L11V, A14P, | NC | N82bS, N83R, V89L | R105Q |
| | D23A | | | |
| 19 | L11V, A14P | H37Y, | N82bS, N83R, V89L | R105Q |
| 20 | L11V, A14P | G40A | N82bS, N83R, V89L, | R105Q |
| | L11V, A14P | A41P | N82bS, N83R, V89L | R105Q |
| 21 | L11V, A14P | F47L | N82bS, N83R, V89L, | R105Q |
| 22 | L11V, A14P | NC | N82bS, N83R, V89L, E93N | R105Q |
| 23 | L11V, A14P | NC | N82bS, N83R, V89L | R105Q |
| 24 | L11V, A14P, | H37Y, G40A, | N73A, N82bS, N83R, | R105Q |
| | D23A | A41P | V89L | |
| 25 | L11V, A14P, | H37Y, G40A, | N73Y, N82bS, N83R, | R105Q |
| | D23A | A41P | V89L | |
| 26 | L11V, A14P, | H37Y, G40A, | N73Q, N82bS, N83R, | R105Q |
| | D23A | A41P | V89L | |
| 27 | L11V, A14P, | H37Y, G40A, | N82bS, N83R, V89L | R105Q |
| | D23A | A41P | | |
| 28 | L11V, A14P, | H37Y, G40A, | N73Q, N82bS, N83R, | R105Q |
| | D23A | A41P | V89L | |
| 29 | L11V, | NC | S68T, T79Y, R81Q, S82aN, | NC |
| | | | N82bS, K83R, G88A, | |
| | | | V89L | |
| 30 | L11V, | NC | S68T, M77T, T79Y, R81Q, | NC |
| | | | S82aN, N82bS, K83R, | |
| | | | G88A, V89L | |
| 31 | L11V, | NC | S68T, T79Y, R81Q, S82aN, | NC |
| | | | N82bS, K83R, G88A, | |
| | | | V89L, L93N | |
| 32 | L11V, T24A | NC | S68T, T79Y, R81Q, S82aN, | NC |
| | | | N82bS, K83R, G88A, | |
| | | | V89L | |
| 33 | L11V, T25S | NC | S68T, T79Y, R81Q, S82aN, | NC |
| | | | N82bS, K83R, G88A, | |
| | | | V89L | |
| 34 | L11V | R39Q | S68T, T79Y, R81Q, S82aN, | NC |
| | | | N82bS, K83R, G88A, | |
| | | | V89L | |
| 35 | L11V | V40A | S68T, T79Y, R81Q, S82aN, | NC |
| | | | N82bS, K83R, G88A, | |
| | | | V89L | |
| 36 | L11V | NC | F62S, S68T, T79Y, R81Q, | NC |
| | | | S82aN, N82bS, K83R, | |
| | | | G88A, V89L | |
| 37 | L11V | NC | A63V, S68T, T79Y, R81Q, | NC |
| | | | S82aN, N82bS, K83R, | |
| | | | G88A, V89L | |
| 38 | | NC | L11V, S68T, K76N, T79Y, | NC |
| | | | R81Q, S82aN, N82bS, | |
| | | | K83R, G88A, V89L | |
| 39 | L11V | E44Q | S68T, T79Y, R81Q, S82aN, | NC |
| | | | N82bS, K83R, G88A, | |
| | | | V89L | |
| 40 | L11V | NC | K83R, V89L | NC |
| 41 | L11V | NC | S68T, K83R, V89L | NC |
| 42 | L11V | NC | M77T, K83R, V89L | NC |
| 43 | L11V | NC | T79Y, K83R, V89L | NC |
| 44 | L11V | NC | R81Q, K83R, V89L | NC |
| 45 | L11V | NC | S82aN, K83R, V89L | NC |
| 46 | L11V | NC | N82bS, K83R, V89L | NC |
| 47 | L11V | NC | K83R, G88A, V89L | NC |
| 48 | L11V, T24A, | V40A, E44Q | F62S, S68T, M77T, T79Y, | NC |
| | T25S | | R81Q, S82aN, N82bS, | |
| | | | K83R, G88A, V89L | |
| 49 | L11V, T24A, | V40A, E44Q | F62S, S68T, M77T, T79Y, | NC |
| | T25S | | R81Q, S82aN, N82bS, | |
| | | | K83R, G88A, V89L | |
| 50 | L11V, T24A, | V40A, E44Q | F62S, S68T, M77T, T79Y, | NC |
| | T25S | | R81Q, S82aN, N82bS, | |
| | | | K83R, G88A, V89L | |
| 51 | L11V, T24A, | V40A, E44Q | F62S, S68T, M77T, T79Y, | NC |
| | T25S | | R81Q, S82aN, N82bS, | |
| | | | K83R, G88A, V89L | |
| 52 | L11V, T24A, | V40A, E44Q | F62S, S68T, M77T, T79Y, | NC |
| | T25S | | R81Q, S82aN, N82bS, | |
| | | | K83R, G88A, V89L | |
| 53 | L11V, T24A, | V40A, E44Q | F62S, S68T, M77T, T79Y, | NC |
| | T25S | | R81Q, S82aN, N82bS, | |
| | | | K83R, G88A, V89L | |
| 54 | L11V, T24A, | V40A, E44Q, | F62S, S68T, M77T, T79Y, | NC |
| | T25S | | R81Q, S82aN, N82bS, | |
| | | | K83R, G88A, V89L | |
| 55 | L11V, T24A, | V40A, E44Q | F62S, S68T, M77T, T79Y, | NC |
| | T25S | | R81Q, S82aN, N82bS, | |
| | | | K83R, G88A, V89L | |
| 56 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 57 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 58 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 59 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 60 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 61 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 62 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 63 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 64 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 65 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 66 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 67 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 68 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 69 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 70 | L11V, T24A, | V40A, E44Q | F62S, A63V, S68T, M77T, | NC |
| | T25S | | T79Y, R81Q, S82aN, | |
| | | | N82bS, K83R, G88AV89L | |
| 71 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 72 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 73 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 74 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 75 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 76 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 77 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 78 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 79 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 80 | L11V, T24A, | V40A, E44Q | F62S, S68T, M77T, T79Y, | NC |
| | T25S | | R81Q, S82aN, N82bS, | |
| | | | K83R, G88A, V89L | |
| 81 | L11V, T24A, | R39Q, V40A, | F62S, S68T, M77T, T79Y, | NC |
| | T25S | E44Q | R81Q, S82aN, N82bS, | |
| | | | K83R, G88A, V89L | |
| 82 | L11V, T24A, | V40A, E44Q | F62S, A63V, S68T, M77T, | NC |
| | T25S | | T79Y, R81Q, S82aN, | |
| | | | N82bS, K83R, G88A, | |
| | | | V89L | |
| 83 | L11V, T24A, | R39Q, V40A, | F62S, A63V, S68T, M77T, | NC |
| | T25S | E44Q | T79Y, R81Q, S82aN, | |
| | | | N82bS, K83R, G88A, | |
| | | | V89L | |
| 84 | | | N93R | |
| 85 | L11V, A12V | R39Q | R76_V78insT, T83R, | NC |
| | | | V89L, N93R | |
| 86 | L11V, A12V | R39Q | T83R, V89L, N93R | NC |
| 87 | L11V, A12V | R39Q | T60A, T83R, V89L, N93R | NC |
| 88 | L11V, A12V | R39Q | G73N, T83R, V89L, N93R | NC |
| 89 | L11V, A12V | R39Q | R76N, T83R, V89L, N93R | NC |
| 90 | L11V, A12V | R39Q | W78V, T83R, V89L, N93R | NC |
| 91 | L11V, A12V | R39Q | S79Y, T83R, V89L, N93R | NC |
| 92 | L11V, A12V | R39Q | T60A, G73N, R76N, | NC |
| | | | W78V, S79Y, T83R, V89L, | |
| | | | N93R | |
| 93 | L11V, A12V | R39Q | T60A, G73N, W78V, | NC |
| | | | S79Y, T83R, V89L, N93R | |
| 94 | L11V, A12V | R39Q | T60A, W78V, S79Y, T83R, | NC |
| | | | V89L, N93R | |
| 95 | L11V, A12V | R39Q | T60A, R76N, W78V, S79Y, | NC |
| | | | T83R, V89L, N93R | |
| 96 | L11V, A12V | R39Q | T60A, G73A, W78V, | NC |
| | | | S79Y, T83R, V89L, N93R | |
| 97 | L11V, A12V | R39Q | T60A, G73R, W78V, S79Y, | NC |
| | | | T83R, V89L, N93R | |
| 98 | L11V, A12V | R39Q | T60A, W78V, S79Y, T83R, | NC |
| | | | V89L, N93R, | |
| 99 | L11V, A12V | R39Q | T60A, G73A, W78V, | NC |
| | | | S79Y, T83R, V89L, N93R | |
| 100 | L11V, A12V | R39Q | T60A, G73R, W78V, S79Y, | NC |
| | | | T83R, V89L, N93R, | |
| 101 | L11V, A12V | R39Q | T60A, W78V, S79Y, T83R, | NC |
| | | | V89L, N93R | |
| 102 | L11V, A12V | R39Q | T60A, G73A, W78V, | NC |
| | | | S79Y, T83R, V89L, N93R | |
| 103 | L11V, A12V | R39Q | T60A, G73R, W78V, S79Y, | NC |
| | | | T83R, V89L, N93R | |
| 104 | L11V, A12V | R39Q | T60A, W78V, S79Y, T83R, | NC |
| | | | V89L, N93R | |
| 105 | L11V, A12V | R39Q | T60A, G73A, W78V, | NC |
| | | | S79Y, T83R, V89L, N93R | |
| 106 | L11V, A12V | R39Q | T60A, G73A, W78V, | NC |
| | | | S79Y, T83R, V89L, N93R | |
| 107 | L11V, A12V | R39Q | T60A, G73R, W78V, S79Y, | NC |
| | | | T83R, V89L, N93R | |
| 108 | L11V, A12V | R39Q | T60A, W78V, S79Y, T83R, | NC |
| | | | V89L, N93R | |
| 109 | L11V, A12V | R39Q | T60A, G73A, W78V, | NC |
| | | | S79Y, T83R, V89L, N93R, | |
| 110 | L11V, A12V | R39Q | T60A, G73R, W78V, S79Y, | NC |
| | | | T83R, V89L, N93R | |
| 111 | L11V, A12V | R39Q | T60A, G73R, W78V, S79Y, | NC |
| | | | T83R, V89L, N93R | |
| 112 | L11V, A12V | R39Q | T60A, W78V, S79Y, T83R, | NC |
| | | | V89L, N93R, | |
| 113 | L11V, A12V | R39Q | T60A, G73A, W78V, | NC |
| | | | S79Y, T83R, V89L, N93R | |
| 114 | L11V, A12V | R39Q | T60A, G73R, W78V, S79Y, | NC |
| | | | T83R, V89L, N93R, | |
| 115 | L11V, A12V | R39Q | T60A, W78V, S79Y, T83R, | NC |
| | | | V89L, N93R | |
| 116 | L11V, A12V | R39Q | T60A, G73A, W78V, | NC |
| | | | S79Y, T83R, V89L, N93R, | |
| 117 | L11V, A12V | R39Q | T60A, G73R, W78V, S79Y, | NC |
| | | | T83R, V89L, N93R | |
| 118 | L11V, A12V | R39Q | T60A, W78V, S79Y, T83R, | NC |
| | | | V89L, N93R | |
| 119 | L11V, A12V | R39Q | T60A, G73R, W78V, S79Y, | NC |
| | | | T83R, V89L, N93R | |
| 120 | L11V, A12V | R39Q | T60A, G73R, W78V, S79Y, | NC |
| | | | T83R, V89L, N93R | |
| 121 | L11V, A12V | R39Q | T60A, G73R, W78V, S79Y, | NC |
| | | | T83R, V89L, N93R | |
| 122 | L11V, A12V | R39Q | T60A, D72G, W78V, | NC |
| | | | S79Y, T83R, V89L, N93R | |
| 123 | L11V, A12V | R39Q | T60A, D72G, W78V, | NC |
| | | | S79Y, T83R, V89L, N93R | |
| 124 | L11V, A12V | R39Q | T60A, D72Q, W78V, | NC |
| | | | S79Y, T83R, V89L, N93R | |
| 125 | L11V, A12V | R39Q | T60A, D72Q, W78V, | NC |
| | | | S79Y, T83R, V89L, N93R | |

NC—no substitutions and/or insertions; ins—insertion, e.g., R76_V78insT means an insertion between the R at position 76 and the V at position 78; the position numbers are according to the Kabat numbering scheme and the junction between the frameworks and the CDRs are determined according to the AbM numbering scheme.

In a further embodiment of the invention, the Nav1.7 binder comprises an amino acid sequence selected from the group consisting of SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55.

In a further embodiment of the invention, the Nav1.7 binder comprises an amino acid sequence selected from the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81.

In a further embodiment of the invention, the Nav1.7 binder comprises an amino acid sequence selected from the group consisting of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, and SEQ ID NO: 97.

In a further embodiment of the invention, the Nav1.7 binder comprises an amino acid sequence selected from the group consisting of SEQ ID NO: 98, SEQ ID NO: 99, SEQ ID NO: 100, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, and SEQ ID NO: 153.

In a further embodiment of the invention, the Nav1.7 binder comprises an amino acid sequence selected from the group consisting of SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, and SEQ ID NO: 195.

In a further embodiment of the invention, the Nav1.7 binder comprises the amino acid sequence set forth in SEQ ID NO: 96.

In a further embodiment of the invention, the Nav1.7 binder comprises the amino acid sequence set forth in SEQ ID NO: 148.

In a further embodiment of the invention, the Nav1.7 binder comprises the amino acid sequence set forth in SEQ ID NO: 192.

In particular embodiments of the Nav1.7 binders, the N-terminal Glu is substituted with Asp.

Nav1.7 binders of the invention can be fused or linked to one or more other amino acid sequences, chemical entities or moieties by a peptide or non-peptide linker. These other amino acid sequences, chemical entities or moieties can confer one or more desired properties to the resulting Nav1.7 binders of the invention, for example, to provide the resulting Nav1.7 binders of the invention with affinity against another therapeutically relevant target such that the resulting polypeptide becomes “bispecific” with respect to Nav1.7 and that other therapeutically relevant target), or to provide a desired half-life, to provide a cytotoxic effect and/or to serve as a detectable tag or label. Some non-limiting examples of such other amino acid sequences, chemical entities or moieties are:
one or more suitable peptide or polypeptide linkers (such as a 9GS, 15GS or 35GS linker (any combination of 9, 15, 20 or 35 G and S amino acids such as, for example, GGGGSGGGS (9GS linker; SEQ ID NO: 243), GGGGSGGGGSGGGGSGGGGS (20GS linker; SEQ ID NO: 244) or GGGGSGGGGSGGGGSGGGGSGGGGSGGGGSGGGGS (35GS linker; SEQ ID NO: 245)), GGGGSGGGGSGGGGSGGGGSGGGGSGGGGSGGGGSGGGGSGGGGSGGGGS (50GS linker; SEQ ID NO: 463) or (GGGGS (SEQ ID NO: 246)) n wherein n is 1, 2, 3,4, 5, 6, 7, 8, 9 or 10); and/or
one or more binding moieties, directed against a target other than Nav1.7 or epitope thereof, for example, against a different epitope of Nav1.7α, Nav1.1α, Nav1.2α, Nav1.3α, Nav1.4α, Nav1.5α, Nav1.6α, Nav1.8α, Nav1.9α, Na_{X} alpha subunit, a sodium channel beta subunit (e.g., Navβ1, Navβ2, Navβ3, or Navβ4), a calcium channel or a potassium channel); and/or
one or more binding domains or binding units that provide for an increase in half-life (for example, a binding domain or binding unit that can bind against a serum protein such as serum albumin, e.g., human serum albumin), e.g., ALB11002; See WO200868280; WO2006122787 or WO2012175400 and/or
a binding domain, binding unit or other chemical entity that allows for the Nav1.7 binder (e.g., an ISVD such as a Nanobody® ISVD) to be internalized into a desired cell (for example, an internalizing anti-EGFR Nanobody® molecule as described in WO05044858); and/or
a chemical moiety that improves half-life such as a suitable polyethyleneglycol group (i.e. PEGylation) or an amino acid sequence that provides for increased half-life such as human serum albumin or a suitable fragment thereof (i.e. albumin fusion); and/or
a payload such as a cytotoxic payload; and/or
a detectable label or tag, such as a radiolabel or fluorescent label; and/or
a tag that can help with immobilization, detection and/or purification of the binder (e.g., an ISVD such as a Nanobody® ISVD), such as a HISₙ, wherein n is 6 to 18, or FLAG tag or combination thereof (e.g., SEQ ID NO: 56);
a tag that can be functionalized, such as a C-terminal GGC tag; and/or
a C-terminal extension X₍ₙ₎ (e.g., -Ala), which may be as further described herein for the Nav1.7 binders (e.g., an ISVD such as a Nanobody® ISVD) of the invention and/or as described in WO12175741 or WO2015173325.

### Sodium Channel Beta Subunit (Navβ) Binders

The present invention further provides ISVDs that bind the Navβ1 or Navβ2 subunits. These Navβ binders comprise three CDRs having amino acid sequences selected from the table below. The CDR amino acid sequences shown in Table 5 are set forth according to the AbM numbering scheme for defining CDR amino acid sequences. A particular CDR amino acid sequence defined by any one of the other schemes advanced for defining CDR amino acid sequences (See Table 1) may have more or less amino acids than shown for CDR amino acid sequences identified according to the AbM numbering scheme but will overlap the CDR amino acid sequences defined according the AbM numbering scheme. Thus, the CDR amino acid sequences shown herein are not to be construed as limiting and any Navβ binder in which the CDR amino acid sequences have been defined by any other numbering scheme will fall within the scope of the Navβ binders of the present invention provided the amino acid sequences for such Navβ binders comprise the amino acid sequences defined for the three CDR amino acid sequences as shown in Table 5. Thus, regardless of the method used to define the CDRs of a Navβ binder (e.g., Kabat, AbM, Clothia, IMGT, Contact, etc.), any Navβ binder that comprises the three amino acid sequences defined for CDR1, CDR2, and CDR3 for any of the Navβ binders shown in Table 5 are Navβ binders of the present invention.

The Navβ binders comprise three CDRs and four Frameworks (FR) in the following alignment FR1-CDR1-FR2-CDR2-FR3-CDR3-FR4. The Navβ binder CDRs may comprise CDRs comprising the following amino acid sequences.

TABLE 5

| Navß1 binder | CDR1 | CDR2 | CDR3 |
|---|---|---|---|
| F0103478E09 | GRAFSTLAMG | ISRNGNNS | ISTPSASHPYVRKESYRY |
| | (SEQ ID NO: 425) | (SEQ ID NO: 426) | (SEQ ID NO: 427) |
| F0103495F09 | GRALSTY AMG | RISRSGITT | DASTNPAGYYLRNRYDY |
| | (SEQ ID NO: 437) | (SEQ ID NO: 438) | (SEQ ID NO: 439) |
| Navß2 binder | CDR1 | CDR2 | CDR3 |
| F0103240B04 | GGTGRRYAMGW | AIRWSAMTY | TWDYFKYDQVRAYRG |
| | (SEQ ID NO: 422) | (SEQ ID NO: 423) | (SEQ ID NO: 424) |
| F0103492E09 | KSILSFAYMR | SIAIGGATS | PAGQYR |
| | (SEQ ID NO: 428) | (SEQ ID NO: 429) | (SEQ ID NO: 430) |
| F0103500E09 | GRTFSRYQMG | YISWSGSTR | GTAGIISSRPETYDS |
| | (SEQ ID NO: 431) | (SEQ ID NO: 432) | (SEQ ID NO: 433) |
| F0103505D8 | GRTSDLSTMN | RITRRGSTY | ASEMGYHYR |
| | (SEQ ID NO: 434) | (SEQ ID NO: 435) | (SEQ ID NO: 436) |

In particular embodiments of the invention, the Navβ1 binder comprises a CDR1 comprising the amino acid sequence set forth in SEQ ID NO: 425, a CDR2 comprising the amino acid sequence set forth in SEQ ID NO: 426, and a CDR3 comprising the amino acid sequence set forth in SEQ ID NO: 427.

In particular embodiments of the invention, the Navβ1 binder comprises a CDR1 comprising the amino acid sequence set forth in SEQ ID NO: 437, a CDR2 comprising the amino acid sequence set forth in SEQ ID NO: 438, and a CDR3 comprising the amino acid sequence set forth in SEQ ID NO: 439.

In particular embodiments of the invention, the Navβ2 binder comprises a CDR1 comprising the amino acid sequence set forth in SEQ ID NO: 422, a CDR2 comprising the amino acid sequence set forth in SEQ ID NO: 423, and a CDR3 comprising the amino acid sequence set forth in SEQ ID NO: 424.

In particular embodiments of the invention, the Navβ2 binder comprises a CDR1 comprising the amino acid sequence set forth in SEQ ID NO: 428, a CDR2 comprising the amino acid sequence set forth in SEQ ID NO: 429, and a CDR3 comprising the amino acid sequence set forth in SEQ ID NO: 430.

In particular embodiments of the invention, the Navβ2 binder comprises a CDR1 comprising the amino acid sequence set forth in SEQ ID NO: 431, a CDR2 comprising the amino acid sequence set forth in SEQ ID NO: 432, and a CDR3 comprising the amino acid sequence set forth in SEQ ID NO: 433.

In particular embodiments of the invention, the Navβ2 binder comprises a CDR1 comprising the amino acid sequence set forth in SEQ ID NO: 434, a CDR2 comprising the amino acid sequence set forth in SEQ ID NO: 435, and a CDR3 comprising the amino acid sequence set forth in SEQ ID NO: 436.

As recited above, the Navβ1 or Navβ2 binders comprise four frameworks: FR1, FR2, FR3, and FR4 wherein the Navβ1 or Navβ2 binder is a single polypeptide having the structure beginning from the N-terminus FR1-CDR1-FR2-CDR2-FR3-CDR3-FR4. The numbering of the frameworks may be as shown herein be according to the Kabat numbering scheme and the junction between each framework and CDR may be determined by the AbM numbering scheme as shown herein. In particular embodiments, the Nav1.7 binders comprise the VHH2-consensus frameworks FR1, FR2, FR3, and FR4, wherein FR1 has the amino acid sequence set forth in SEQ ID NO: 268, FR2 has the amino acid sequence set forth in SEQ ID NO: 269, FR3 has the amino acid sequence set forth in SEQ ID NO: 270, and FR4 has the amino acid sequence set forth in SEQ ID NO: 271. In further embodiments, each framework may comprise one or more substitutions and or insertions with the proviso that the Navβ1 or Navβ2 binder is capable of binding human Nav1.7α. In further embodiments, frameworks may comprise one or more of the substitutions and/or insertions shown in Table 4 in any combination. In further embodiments, FR1 may comprise one or more of the substitutions shown for FR1 in Table 4. In further embodiments, FR2 may comprise one or more of the substitutions shown for FR2 in Table 4. In further embodiments, FR3 may comprise one or more of the substitutions shown for FR3 in Table 4. In further embodiments, FR4 may comprise one of the substitutions shown for FR4 in Table 4. In a further embodiment, each framework comprises at least one amino acid substitution. In a further embodiment, the Navβ1 or Navβ2 binder comprises at least one substitution and/or insertion shown in Table 4 for each of FR1, FR2, FR3, and FR4. In a further embodiment, the Navβ1 or Navβ2 binder comprises the one substitution or specific substitution and/or insertion combination shown in Table 4 for each of FR1, FR2, FR3, and FR4. In a further embodiment, FR1 comprises at least an L11V substitution and FR3 comprises at least a V89L substitution. In a further still embodiment, the Navβ1 or Navβ2 binder may comprise one of the 125 specific sets of FR1, FR2, FR3, and FR4 combinations shown in Table 4. In any one of the above embodiments, the FR1 may further comprise a Q1E or a Q1D amino acid substitution.

In particular embodiments of the invention, the Navβ1 binder comprises the amino acid sequence set forth in SEQ ID NO: 411.

In particular embodiments of the invention, the Navβ1 binder comprises the amino acid sequence set forth in SEQ ID NO: 415.

In particular embodiments of the invention, the Navβ2 binder comprises the amino acid sequence set forth in SEQ ID NO: 410.

In particular embodiments of the invention, the Navβ2 binder comprises the amino acid sequence set forth in SEQ ID NO: 412.

In particular embodiments of the invention, the Navβ2 binder comprises the amino acid sequence set forth in SEQ ID NO: 413.

In particular embodiments of the invention, the Navβ2 binder comprises the amino acid sequence set forth in SEQ ID NO: 414.

The Navβ binders of the invention can be fused or linked to one or more other amino acid sequences, chemical entities or moieties by a peptide or non-peptide linker. These other amino acid sequences, chemical entities or moieties can confer one or more desired properties to the resulting Navβ binders of the invention, for example, to provide the resulting Navβ binders of the invention with affinity against another therapeutically relevant target such that the resulting polypeptide becomes “bispecific” with respect to Navβ and that other therapeutically relevant target), or to provide a desired half-life, to provide a cytotoxic effect and/or to serve as a detectable tag or label. Some non-limiting examples of such other amino acid sequences, chemical entities or moieties are:
one or more suitable peptide or polypeptide linkers (such as a 9GS, 15GS or 35GS linker (any combination of 9, 15, 20 or 35 G and S amino acids such as, for example, GGGGSGGGS (9GS linker; SEQ ID NO: 243), GGGGSGGGGSGGGGSGGGGS (20GS linker; SEQ ID NO: 244) or GGGGSGGGGSGGGGSGGGGSGGGGSGGGGSGGGGS (35GS linker; SEQ ID NO: 245)), GGGGSGGGGSGGGGSGGGGSGGGGSGGGGSGGGGSGGGGSGGGGSGGGGS (50GS linker; SEQ ID NO: 463), or (GGGGS (SEQ ID NO: 246)) n wherein n is 1, 2, 3,4, 5, 6, 7, 8, 9 or 10); and/or
one or more binding moieties, directed against a target other than Navβ or epitope thereof, for example, against a different epitope of Navβ, Nav1.1α, Nav1.2α, Nav1.3α, Nav1.4α, Nav1.5α, Nav1.6α, Nav1.7α, Nav1.8α, Nav1.9α, Na_{X} alpha subunit, a sodium channel beta subunit (e.g., Navβ1, Navβ2, Navβ3, or Navβ4), a calcium channel or a potassium channel); and/or
one or more binding domains or binding units that provide for an increase in half-life (for example, a binding domain or binding unit that can bind against a serum protein such as serum albumin, e.g., human serum albumin), e.g., ALB11002; See WO200868280; WO2006122787 or WO2012175400 and/or
a binding domain, binding unit or other chemical entity that allows for the Navβ binder (e.g., an ISVD such as a Nanobody® ISVD) to be internalized into a desired cell (for example, an internalizing anti-EGFR Nanobody® molecule as described in WO05044858); and/or a chemical moiety that improves half-life such as a suitable polyethyleneglycol group (i.e. PEGylation) or an amino acid sequence that provides for increased half-life such as human serum albumin or a suitable fragment thereof (i.e. albumin fusion); and/or
a payload such as a cytotoxic payload; and/or
a detectable label or tag, such as a radiolabel or fluorescent label; and/or
a tag that can help with immobilization, detection and/or purification of the binder (e.g., an ISVD such as a Nanobody® ISVD, such as a HISₙ, wherein n is 6 to 18, or FLAG tag or combination thereof (e.g., SEQ ID NO: 56);
a tag that can be functionalized, such as a C-terminal GGC tag; and/or
a C-terminal extension X₍ₙ₎ (e.g., -Ala), which may be as further described herein.

### Nav1.7-Navβ Bispecific Binders

The present invention further provides Nav1.7-Navβ bispecific binders comprising at least one Nav1.7 binder and at least one Navβ binder linked together by peptide or polypeptide linker. As used herein, Nav1.7-Navβ bispecific binder refers to binders comprising one or more Nav1.7 binders linked to one or more Navβ binders. In an embodiment, the Nav1.7-Navβ bispecific binders comprise a Nav1.7 ISVD linked via a peptide or polypeptide linker at the C-terminus of the Nav1.7 ISVD to the N-terminus of a Navβ ISVD. In another embodiment, the Nav1.7-Navβ bispecific binders comprise a Navβ ISVD linked via a peptide or polypeptide linker at the C-terminus of the Navβ ISVD to the N-terminus of a Nav1.7 ISVD. The Nav1.7-Navβ bispecific binders are provided as a continuous amino acid sequence.

In particular embodiments, the peptide or polypeptide linker comprises repeating Gly (G) and Ser (S) amino acids to provide for example, 9GS, 15GS, or 35GS peptide or polypeptide linkers (any combination of 9, 15, 20 or 35 G and S amino acids such as, for example, GGGGSGGGS (9GS linker; SEQ ID NO: 243), GGGGSGGGGSGGGGSGGGGS (20GS linker; SEQ ID NO: 244) or GGGGSGGGGSGGGGSGGGGSGGGGSGGGGSGGGGS (35GS linker; SEQ ID NO: 245)), GGGGSGGGGSGGGGSGGGGSGGGGSGGGGSGGGGSGGGGSGGGGSGGGGS (50GS linker; SEQ ID NO: 463), or (GGGGS (SEQ ID NO: 246)) n wherein n is 1, 2, 3,4, 5, 6, 7, 8, 9 or 10).

In particular embodiments, the N-terminal amino acid of the Nav1.7-Navβ bispecific binders is an Asp or Glu amino acid and the C-terminus of the Nav1.7-Navβ bispecific binders comprises a C-terminal extension of one or more Ala amino acids. In particular embodiments, the C-terminal extension consists of one Ala residue.

In particular embodiments of the Nav1.7-Navβ1 bispecific binder, the Navβ binder is a Navβ1 binder or a Navβ2 binder.

In particular embodiments, the Nav1.7-Navβ1 bispecific binder comprises a Navβ1 binder comprising (a) a CDR1 comprising the amino acid sequence set forth in SEQ ID NO: 425, a CDR2 comprising the amino acid sequence set forth in SEQ ID NO: 426, and a CDR3 comprising the amino acid sequence set forth in SEQ ID NO: 427; or (b) a CDR1 comprising the amino acid sequence set forth in SEQ ID NO: 437, a CDR2 comprising the amino acid sequence set forth in SEQ ID NO: 438, and a CDR3 comprising the amino acid sequence set forth in SEQ ID NO: 439.

In a further embodiment, the Nav1.7-Navβ1 bispecific binder comprises a Navβ1 binder comprising the amino acid sequence set forth in SEQ ID NO: 411 or the amino acid sequence set forth in SEQ ID NO: 415.

In particular embodiments, the Nav1.7-Navβ2 bispecific binder comprises a Navβ2 binder comprising (a) a CDR1 comprising the amino acid sequence set forth in SEQ ID NO: 422, a CDR2 comprising the amino acid sequence set forth in SEQ ID NO: 423, and a CDR3 comprising the amino acid sequence set forth in SEQ ID NO: 424; (b) a CDR1 comprising the amino acid sequence set forth in SEQ ID NO: 428, a CDR2 comprising the amino acid sequence set forth in SEQ ID NO: 429, and a CDR3 comprising the amino acid sequence set forth in SEQ ID NO: 430; (c) a CDR1 comprising the amino acid sequence set forth in SEQ ID NO: 431, a CDR2 comprising the amino acid sequence set forth in SEQ ID NO: 432, and a CDR3 comprising the amino acid sequence set forth in SEQ ID NO: 433; or (d) a CDR1 comprising the amino acid sequence set forth in SEQ ID NO: 434, a CDR2 comprising the amino acid sequence set forth in SEQ ID NO: 435, and a CDR3 comprising the amino acid sequence set forth in SEQ ID NO: 436.

In a further embodiment, the Nav1.7-Navβ1 bispecific binder comprises a Navβ2 binder comprising the amino acid sequence set forth in SEQ ID NO: 410, the amino acid sequence set forth in SEQ ID NO: 412, the amino acid sequence set forth in SEQ ID NO: 413, or amino acid sequence set forth in SEQ ID NO: 414.

In particular embodiments, the Nav1.7-Navβ1 bispecific binder comprises a Nav1.7 binder comprising (a) a CDR1 comprising the amino acid sequence set forth in SEQ ID NO: 196 or SEQ ID NO: 197; a CDR2 comprising the amino acid sequence set forth in SEQ ID NO: 198 or SEQ ID NO: 199; and, a CDR3 comprising the amino acid sequence set forth in SEQ ID NO: 200; (b) a CDR1 comprising the amino acid sequence set forth in SEQ ID NO: 201; a CDR2 comprising the amino acid sequence set forth in SEQ ID NO: 202, SEQ ID NO: 203, SEQ ID NO: 204, or SEQ ID NO: 205; and, a CDR3 comprising the amino acid sequence set forth in SEQ ID NO: 206; (c) a CDR1 comprising the amino acid sequence set forth in SEQ ID NO: 207, SEQ ID NO: 208, SEQ ID NO: 209, SEQ ID NO: 210, SEQ ID NO: 211, or SEQ ID NO: 212; a CDR2 comprising the amino acid sequence set forth in SEQ ID NO: 213, SEQ ID NO: 214, SEQ ID NO: 215, SEQ ID NO: 216, SEQ ID NO: 217, or SEQ ID NO: 218; and a CDR3 comprising the amino acid sequence set forth in SEQ ID NO: 219; (d) a CDR1 comprising the amino acid sequence set forth in SEQ ID NO: 201 or SEQ ID NO: 222; a CDR2 comprising the amino acid sequence set forth in SEQ ID NO: 223 or SEQ ID NO: 224; and a CDR3 comprising the amino acid sequence set forth in SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, or SEQ ID NO: 233; (e) a CDR1 comprising the amino acid sequence set forth in SEQ ID NO: 201; a CDR2 comprising the amino acid sequence set forth in SEQ ID NO: 205; and, a CDR3 comprising the amino acid sequence set forth in SEQ ID NO: 206; (0 a CDR1 comprising the amino acid sequence set forth in SEQ ID NO: 211; a CDR2 comprising the amino acid sequence set forth in SEQ ID NO: 215; and, a CDR3 comprising the amino acid sequence set forth in SEQ ID NO: 219; or (g) a CDR1 comprising the amino acid sequence set forth in SEQ ID NO: 222; a CDR2 comprising the amino acid sequence set forth in SEQ ID NO: 223; and, a CDR3 comprising the amino acid sequence set forth in SEQ ID NO: 233.

In any one of the foregoing embodiments, the Nav1.7 binder comprising the Nav1.7-Navβ bispecific binder comprises (a) an amino acid sequence selected from the group consisting of SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55; (b) an amino acid sequence selected from the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81; (c) an amino acid sequence selected from the group consisting of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, and SEQ ID NO: 97; (d) an amino acid sequence selected from the group consisting of SEQ ID NO: 98, SEQ ID NO: 99, SEQ ID NO: 100, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, and SEQ ID NO: 153; (e) an amino acid sequence selected from the group consisting of SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, and SEQ ID NO: 195.

In particular embodiments, the Nav1.7 binder comprising the Nav1.7-Navβ bispecific binder comprises the amino acid sequence set forth in SEQ ID NO: 96; the amino acid sequence set forth in SEQ ID NO: 148; or, the amino acid sequence set forth in SEQ ID NO: 192.

In particular embodiments of the Nav1.7 binders or Navβ binders comprising the Nav1.7-Navβ bispecific binder, the N-terminal Glu is substituted with Asp. In particular embodiments, the N-terminal ISVD of the Nav1.7-Navβ binder comprises an Asp amino acid residue at the N-terminus.

### Half-Life Extenders (HLE)

The Nav1.7 binders, Navβ binders, and Nav1.7-Navβ bispecific binders of the present invention, may further comprise one or more half-life extenders such as one or more anti-HSA (human serum albumin) binders and/or one or more polyethylene glycol (PEG) molecules.

As discussed herein, the “HSA binders” of the present invention bind to HSA (e.g., an ISVD such as a Nanobody® ISVD) as well as any binder which includes such a molecule that is fused to another binder. An individual HSA binder may be referred to as an HSA binding moiety if it is part of a larger molecule, e.g., a multivalent molecule.

As further described herein, the HSA binders of the invention that are fused to the Nav1.7 binder, Navβ binder, or Nav1.7-Navβ bispecific binder comprise the same combination of CDRs (i.e., CDR1, CDR2 and CDR3) as are present in ALB11002 or comprise the amino acid sequence of ALB11002 (SEQ ID NO: 234).

The present invention also includes Nav1.7 binders, Navβ binders, and Nav1.7-Navβ bispecific binders that further include being linked by a peptide or polypeptide linker to one or more HSA binding moieties which are variants of ALB11002, e.g., wherein the HSA binder comprises CDR1, CDR2 and CDR3 of said ALB11002 variants set forth below in Table 6.

TABLE 6
Human Serum Albumin (HSA) Binders

| SEQ | | |
|---|---|---|
| ID | | |
| NO: | Description | Sequence |
| 238 | ALB11002 | EVQLVESGGGXVQPGNSLRLSCAASGFTFSSFGMSW |
| | (may be referred | VRQAPGKGLEWVSSISGSGSDTLYADSVKGRFTISRD |
| | to herein as | NAKTTLYLQMNSLRPEDTAXYYCTIGGSLSRSSQGTL |
| | “ALB201”) | VTVSSA; |
| | | wherein X at positions 11 and 93 are each L or V. |
| | | The CDRs are defined according to the AbM numbering |
| | | scheme. |
| 235 | HSA-CDR1 | GFTFSSFGMS |
| 236 | HSA-CDR2 | SISGSGSDTL |
| 237 | HSA-CDR3 | GGSLSR |
| 265 | ALB00223 | EVOLVESGGGVVQPGGSLRLSCAASGFTFRSFGMSW |
| | | VRQAPGKGPEWVSSISGSGSDTLYADSVKGRFTISRD |
| | | NSKNTLYLQMNSLRPEDTALYYCTIGGSLSRSSQGTL |
| | | VTVSSA |
| | | The CDRs are defined according to the AbM numbering |
| | | scheme. |
| 267 | HSA-CDR1 | GFTFRSFGMS |

In particular embodiments, the ALB11002 further lacks the C-terminal Alanine (SEQ ID NO: 234). In a further embodiment, the HSA binder comprises the amino acid sequence set forth in SEQ ID NO: 238 but which further comprises an E1D, V11L, and an L93V substitution to provide an HSA binder comprising the amino acid sequence set forth in SEQ ID NO: 240:

### EVQLVESGGGVVQPGNSLRLSCAASGFTFSSFGMSWVRQAPGKGLEWVSSISGSGSDTL YADSVKGRFTISRDNAKTTLYLQMNSLRPEDTALYYCTIGGSLSRSSQGTLVTVSSA.

This embodiment may further lack the C-terminal Alanine to provide the amino acid sequence set forth in SEQ ID NO: 239.

In an embodiment of the invention, the HLE is ALB11 comprising the amino acid sequence:

EVQLVESGGGLVQPGNSLRLSCAASGFTFS SFGMSWVRQAPGKGLEWVSSISGSGSDTL YADSVKGRFTISRDNAKTTLYLQMNSLRPEDTAVYYCTIGGSLSRSSQGTLVTVSSA (SEQ ID NO: 242) and in a further embodiment lacks the C-terminal Alanine (SEQ ID NO:241).

In particular embodiments ALB00233 lacks a C-terminal A as shown in SEQ ID NO: 266.

In an embodiment of the invention, the half-life extender is an HSA binder comprising: a CDR1 that comprises the amino acid sequence GFTFSSFGMS (SEQ ID NO: 235) or GFTFRSFGMS (SEQ ID NO: 267); a CDR2 that comprises the amino acid sequence SISGSGSDTL (SEQ ID NO: 236); and a CDR3 that comprises the amino acid sequence GGSLSR (SEQ ID NO: 237).

In an embodiment of the invention, the first amino acid of any of the HSA binders is E and in another embodiment of the invention, the first amino acid of any of the HSA binders is D.

In particular embodiments, the peptide or polypeptide linker comprises repeating Gly (G) and Ser (S) amino acids to provide for example, 9GS, 15GS, or 35GS peptide or polypeptide linkers (any combination of 9, 15, 20 or 35 G and S amino acids such as, for example, GGGGSGGGS (9GS linker; SEQ ID NO: 243), GGGGSGGGGSGGGGSGGGGS (20GS linker; SEQ ID NO: 244) or GGGGSGGGGSGGGGSGGGGSGGGGSGGGGSGGGGS (35GS linker; SEQ ID NO: 245)),

GGGGSGGGGSGGGGSGGGGSGGGGSGGGGSGGGGSGGGGSGGGGSGGGGS (50GS linker; SEQ ID NO: 463), or (GGGGS (SEQ ID NO: 246)) n wherein n is 1, 2, 3,4, 5, 6, 7, 8, 9 or 10).

In another embodiment of the invention, the half-life extender is a polyethylene glycol (PEG) moiety appended to the Nav1.7 binder, Navβ binder, or Nav1.7-Navβ bispecific binder to provide a PEGylated Nav1.7 binder, Navβ binder, or Nav1.7-Navβ bispecific binder. The molecular weight of the polyethylene glycol (PEG) moiety may be about 12,000 daltons or about 20,000 daltons. In an embodiment of the invention, the Nav1.7 binder, Navβ binder, or Nav1.7-Navβ bispecific binder comprises one or more polyethylene glycol molecules covalently attached via a linker (e.g., a C₂₋₁₂ alkyl such as —CH₂CH₂CH₂—) to a single amino acid residue of a single subunit of the Nav1.7 binder, Navβ binder, or Nav1.7-Navβ bispecific binder, wherein said amino acid residue is the alpha amino group of the N-terminal amino acid residue or the epsilon amino group of a lysine residue. In an embodiment of the invention, the PEGylated binder is: (PEG)_{b}-L-NH-[binder]; wherein b is 1-9 and L is a C₂₋₁₂ alkyl linker moiety covalently attached to a nitrogen (N) of the single amino acid residue of the binder. In an embodiment of the invention, the PEGylated binder has the formula: [X-0(CH₂CH₂O) ₙ]_{b}-L-NH-[binder], wherein X is H or C₁₋₄ alkyl; n is 20 to 2300; b is 1 to 9; and L is a C₁₋₁₁ alkyl linker moiety which is covalently attached to the nitrogen (N) of the alpha amino group at the amino terminus of one binder subunit; provided that when b is greater than 1, the total of n does not exceed 2300. See, for example, U.S. Pat. No. 7,052,686, which is incorporated herein by reference in its entirety.

To PEGylate a Nav1.7 binder, Navβ binder, or Nav1.7-Navβ bispecific binder, typically the binder is reacted with a reactive form of polyethylene glycol (PEG), such as a reactive ester or aldehyde derivative of PEG, under conditions in which one or more PEG groups become attached to the binder. In particular embodiments, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatizeother proteins, such as mono (C1-C10) alkoxy- or aryloxy-polyethylene glycol or polyethylene glycol-maleimide. In certain embodiments, the binder to be PEGylated is an aglycosylated binder. Methods for PEGylating proteins are known in the art and can be applied to the binder of the invention. See, e.g., EP0154316 and EP0401384, each of which is incorporated herein by reference in its entirety.

In certain embodiments, the Nav1.7 binder, Navβ binder, or Nav1.7-Navβ bispecific binder is fused at the C-terminus to an HC constant domain of Fc domain thereof domain. In a particular embodiment, the HC domain or Fc domain thereof is of the IgG1, IgG2, IgG3, or IgG4 isotype. The amino acid sequences of the IgG1, IgG2, and IgG4 isotype HC constant domains are set forth in SEQ ID NO: 469, SEQ ID NO: 476, and SEQ ID No: 482, respectively. In the embodiments herein, the Fc domain may comprise the CH2 and CH3 domains of the HC constant domain. In particular embodiments, the Fc domain may further comprise the hinge region between the CH1 and CH2 domains or the hinge region comprising one or amino acid deletions. In exemplary embodiments, Nav1.7 binders, Navβ binders, or Nav1.7-Navβ bispecific binders are fused to an HC domain or Fc domain thereof of the IgG1, IgG2, or IgG4 isotype. In particular embodiments, the Nav1.7 binders, Navβ binders, or Nav1.7-Navβ bispecific binders are fused to the N-terminus of an HC domain or Fc domain thereof. In particular embodiments, the Nav1.7 binders, Navβ binders, or Nav1.7-Navβ bispecific binders are fused to the C-terminus of an HC domain or Fc domain thereof.

Nav1.7 binders, Navβ binders, or Nav1.7-Navβ bispecific binders of the present invention further include ISVDs that are fused or linked to an effector-silent HC constant domain or Fc domain thereof The effector-silent HC constant domain or Fc domain has been modified such that it displays no measurable binding to one or more FcRs or displays reduced binding to one or more FcRs compared to that of an unmodified HC constant domain or Fc domain of the same IgG isotype. The effector-silent HC constant domain or Fc domain may in further embodiments display no measurable binding to each of FcγRIIIa, FcγRIIa, and FcγRI or display reduced binding to each of FcγRIIIa, FcγRIIa, and FcγRI compared to that of an unmodified antibody of the same IgG isotype. In particular embodiments, the effector-silent HC constant domain or Fc domain is a modified human HC constant domain or Fc domain.

In particular embodiments, the effector-silent HC constant domain or Fc domain thereof comprises an Fc domain of an IgG1 or IgG2, IgG3, or IgG4 isotype that has been modified to lack N-glycosylation of the asparagine (Asn) residue at position 297 (Eu numbering system) of the HC constant domain. The consensus sequence for N-glycosylation is Asn-Xaa-Ser/Thr (wherein Xaa at position 298 is any amino acid except Pro); in all four isotypes the N-glycosylation consensus sequence is Asn-Ser-Thr. The modification may be achieved by replacing the codon encoding the Asn at position 297 in the nucleic acid molecule encoding the HC constant domain with a codon encoding another amino acid, for example Ala, Asp, Gln, Gly, or Glu, e.g. N297A, N297Q, N297G, N297E, or N297D. Alternatively, the codon for Ser at position 298 may be replaced with the codon for Pro or the codon for Thr at position 299 may be replaced with any codon except the codon for Ser. In a further alternative each of the amino acids comprising the N-glycosylation consensus sequence is replaced with another amino acid. Such modified IgG molecules have no measurable effector function. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations. In further embodiments, such IgGs modified to lack N-glycosylation at position 297 may further include one or more additional mutations disclosed herein for eliminating measurable effector function.

An exemplary IgG1 HC constant domain or Fc domain thereof mutated at position 297, which abolishes the N-glycosylation of the HC constant domain, is set forth in SEQ ID NO: 474, an exemplary IgG2 HC constant domain mutated at position 297, which abolishes the N-glycosylation of the HC constant, is set forth in SEQ ID NO: 480, and an exemplary IgG4 HC constant domain mutated at position 297 to abolish N-glycosylation of the HC constant domain is set forth in SEQ ID NO: 485. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain or Fc domain thereof of the IgG1 IgG2, IgG3, or IgG4 HC constant domain is modified to include one or more amino acid substitutions selected from E233P, L234A, L235A, L235E, N297A, N297D, D265S, and P331S (wherein the positions are identified according to Eu numbering) and wherein said HC constant domain is effector-silent. In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain or Fc domain thereof comprises L234A, L235A, and D265S substitutions (wherein the positions are identified according to Eu numbering). In particular embodiments, the HC constant domain comprises an amino acid substitution at position Pro329 and at least one further amino acid substitution selected from E233P, L234A, L235A, L235E, N297A, N297D, D265S, and P331S (wherein the positions are identified according to Eu numbering). These and other substitutions are disclosed in WO9428027; WO2004099249; WO20121300831, U.S. Pat. Nos. 9,708,406; 8,969,526; 9,296,815; Sondermann et al. Nature 406, 267-273 (2000), each of which is incorporated herein by reference in its entirety).

In particular embodiments of the above, the HC constant domain or Fc domain thereof comprises an L234A/L235A/D265A; L234A/L235A/P329G; L235E; D265A; D265A/N297G; or V234A/G237A/P238S/H268A/V309L/A330S/P331S substitutions, wherein the positions are identified according to Eu numbering. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent HC constant domain or Fc domain thereof comprises an IgG1 isotype, in which the Fc domain of the HC constant domain has been modified to be effector-silent by substituting the amino acids from position 233 to position 236 of the IgG1 with the corresponding amino acids of the human IgG2 HC and substituting the amino acids at positions 327, 330, and 331 with the corresponding amino acids of the human IgG4 HC, wherein the positions are identified according to Eu numbering (Armour et al., Eur. J. Immunol. 29(8):2613-24 (1999); Shields et al., J. Biol. Chem. 276(9):6591-604(2001)). In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent HC constant domain or Fc domain thereof is a hybrid human immunoglobulin HC constant domain, which includes a hinge region, a CH2 domain and a CH3 domain in an N-terminal to C-terminal direction, wherein the hinge region comprises an at least partial amino acid sequence of a human IgD hinge region or a human IgG1 hinge region; and the CH2 domain is of a human IgG4 CH2 domain, a portion of which, at its N-terminal region, is replaced by 4-37 amino acid residues of an N-terminal region of a human IgG2 CH2 or human IgD CH2 domain. Such hybrid human HC constant domain is disclosed in U.S. Pat. No. 7,867,491, which is incorporated herein by reference in its entirety.

In particular embodiments, the effector-silent HC constant domain or Fc domain thereof is an IgG4 HC constant domain in which the serine at position 228 according to the Eu system is substituted with proline, see for example SEQ ID NO: 52. This modification prevents formation of a potential inter-chain disulfide bond between the cysteines at positions Cys226 and Cys229 in the EU numbering scheme and which may interfere with proper intra-chain disulfide bond formation. See Angal et al. Mol. Imunol. 30:105 (1993); see also (Schuurman et al., Mol. Immunol. 38: 1-8, (2001)). In further embodiments, the IgG4 constant domain includes in addition to the S228P substitution, a P239G, D265A, or D265A/N297G amino acid substitution, wherein the positions are identified according to Eu numbering. In particular embodiments of the above, the IgG4 HC constant domain is a human HC constant domain. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

Exemplary IgG1 HC constant domains comprise an amino acid sequence selected from the group consisting of amino acid sequences set forth in SEQ ID NO: 470, SEQ ID NO: 471, SEQ ID NO: 472, SEQ ID NO: 473, SEQ ID NO: 474, and SEQ ID NO: 475.

Exemplary IgG2 HC constant domains comprise an amino acid sequence selected from the group consisting of amino acid sequences set forth in SEQ ID NO: 477, SEQ ID NO: 478, SEQ ID NO: 479, and SEQ ID NO: 480.

Exemplary IgG4 HC constant domains comprise an amino acid sequence selected from the group consisting of amino acid sequences set forth in SEQ ID NO: 483, SEQ ID NO: 484, and SEQ ID NO: 485.

The particular embodiments, the Nav1.7 binder, Navβ binder, or Nav1.7-Navβ bispecific binder is linked to the HC constant domain or Fc domain thereof by a peptide or polypeptide linker to provide a fusion protein comprising the structure binder-linker-HC constant domain or Fc domain thereof or HC constant domain-linker-binder wherein binder refers to Nav1.7 binder, Navβ binder, or Nav1.7-Navβ bispecific binder. The Fc domain thereof as used herein includes embodiments lacking the hinge region and embodiments wherein the Fc comprises one or amino acids of the hinge region.

In particular embodiments, the peptide or polypeptide linker comprises repeating Gly (G) and Ser (S) amino acids to provide for example, 9GS, 15GS, or 35GS peptide or polypeptide linkers (any combination of 9, 15, 20 or 35 G and S amino acids such as, for example, GGGGSGGGS (9GS linker; SEQ ID NO: 243), GGGGSGGGGSGGGGSGGGGS (20GS linker; SEQ ID NO: 244) or GGGGSGGGGSGGGGSGGGGSGGGGSGGGGSGGGGS (35GS linker; SEQ ID NO: 245)),

GGGGSGGGGSGGGGSGGGGSGGGGSGGGGSGGGGSGGGGSGGGGSGGGGS (50GS linker; SEQ ID NO: 463), or (GGGGS (SEQ ID NO: 246)) n wherein n is 1, 2, 3, 4, 5, 6, 7, 8, 9 or 10).

In particular embodiments, the Nav1.7 binders, Navβ binders, or Nav1.7-Navβ bispecific binders are fused to the N-terminus of an effector-silent HC domain or Fc domain thereof. In particular embodiments, the Nav1.7 binders, Navβ binders, or Nav1.7-Navβ bispecific binders are fused to the C-terminus of an effector-silent HC domain or Fc domain thereof.

In particular embodiments, the Nav1.7 binders, Navβ binders, or Nav1.7-Navβ bispecific binders are linked to the N-terminus of an effector-silent HC domain or Fc domain thereof by a non-peptide linker, which in particular embodiments, may be a non-peptide polymer. The non-peptide polymer refers to a biocompatible polymer to which at least two repeat units are conjugated, and the repeat units are interconnected by random covalent bonds other than peptide bonds. The non-peptide polymer may be selected from the group consisting of polyethylene glycol, polypropylene glycol, a copolymer between ethylene glycol and propylene glycol, polyoxyethylated polyol, polyvinyl alcohol, polysaccharide, dextran, polyvinyl ethyl ether, a biodegradable polymer such as polylactic acid (PLA) and polylactic-glycolic acid (PLGA), lipid polymer, chitins, hyaluronic acid, and a combination thereof, and preferably, polyethylene glycol. The derivatives known in the art and the derivatives that can easily be prepared using the technology in the art are also included in the scope of the present invention. In particular embodiments, the non-peptide linker comprises polyethylene glycol, which in particular embodiments may be 3,400 daltons. Conjugates comprising a heterologous protein conjugated to an Fc domain by a non-peptide linker have been disclosed in U.S. Pat. Nos. 7,636,420; 7,737,260; 7,968,316; 8,029,789; 8,110,665; 8,124,094; 8,822,650; 8,846,874; 9,394, 546; 10,071,171; 10,272,159; and 10,973,881, each of which is incorporated herein by reference in its entirety.

In particular embodiments, the HC constant domain or Fc domain conjugates form a homodimer wherein each HC constant domain or Fc domain conjugates comprising the homodimer is fused or conjugated to the same binder selected from Nav1.7 binder, Navβ binder, and Nav1.7-Navβ bispecific binder. In particular embodiments, the HC constant domain or Fc domain conjugates form a heterodimer wherein a HC constant domain or Fc domain conjugate comprising the heterodimer is fused or conjugated to a binder selected from Nav1.7 binder, Navβ binder, and Nav1.7-Navβ bispecific binder and a second HC constant domain or Fc domain conjugate comprising the heterodimer is fused or conjugated to a binder selected from Nav1.7 binder, Navβ binder, and Nav1.7-Navβ bispecific binder that is not fused or conjugated to the first HC constant domain or Fc domain conjugate. In particular embodiments, the HC constant domain or Fc domain conjugate form a heterodimer wherein a first HC constant domain or Fc domain conjugate comprising the heterodimer is fused or conjugated to a binder selected from Nav1.7 binder, Navβ binder, and Nav1.7-Navβ bispecific binder and the second HC constant domain or Fc domain is not fused or conjugated to a Nav1.7 binder, Navβ binder, and Nav1.7-Navβ bispecific binder. In particular embodiments, the second HC constant domain or Fc domain is fused or conjugated to a heterologous protein, which may be the Fab of an antibody or ISVD other than a Nav1.7 binder, Navβ binder, or Nav1.7-Navβ bispecific binder; a heterologous protein, polypeptide, or peptide; or a small molecule. HC constant domain and Fc domain heterodimers have been disclosed in WO9627011; WO9850431; WO9929732; WO2009089004; WO2013055809; WO2013063702; WO2014145907; and WO2014084607, each of which is incorporated herein by reference in its entirety.

In particular embodiments of the invention, the HC constant or Fc domains as disclosed herein may comprise a C-terminal lysine or lack either a C-terminal lysine or a C-terminal glycine-lysine dipeptide.

### C-Terminal Extensions

The present invention further provides Nav1.7 binders, Navβ binders, or Nav1.7-Navβ bispecific binders that comprise a C-terminal extension. The present invention provides, for example, C-terminal extensions such as X(n), wherein X and n can be as follows:
(a) n=1 and X=Ala;
(b) n=2 and each X=Ala;
(c) n=3 and each X=Ala;
(d) n=2 and at least one X=Ala (with the remaining amino acid residue(s) X being independently chosen from any naturally occurring amino acid but preferably being independently chosen from Val, Leu and/or Ile);
(e) n=3 and at least one X=Ala (with the remaining amino acid residue(s) X being independently chosen from any naturally occurring amino acid but preferably being independently chosen from Val, Leu and/or Ile);
(f) n=3 and at least two X=Ala (with the remaining amino acid residue(s) X being independently chosen from any naturally occurring amino acid but preferably being independently chosen from Val, Leu and/or Ile);
(g) n=1 and X=Gly;
(h) n=2 and each X=Gly;
(i) n=3 and each X=Gly;
(j) n=2 and at least one X=Gly (with the remaining amino acid residue(s) X being independently chosen from any naturally occurring amino acid but preferably being independently chosen from Val, Leu and/or Ile);
(k) n=3 and at least one X=Gly (with the remaining amino acid residue(s) X being independently chosen from any naturally occurring amino acid but preferably being independently chosen from Val, Leu and/or Ile);
(l) n=3 and at least two X=Gly (with the remaining amino acid residue(s) X being independently chosen from any naturally occurring amino acid but preferably being independently chosen from Val, Leu and/or Ile);
(m) n=2 and each X=Ala or Gly;
(n) n=3 and each X=Ala or Gly;
(o) n=3 and at least one X=Ala or Gly (with the remaining amino acid residue(s) X being independently chosen from any naturally occurring amino acid but preferably being independently chosen from Val, Leu and/or Ile); or
(p) n=3 and at least two X=Ala or Gly (with the remaining amino acid residue(s) X being independently chosen from any naturally occurring amino acid but preferably being independently chosen from Val, Leu and/or Ile);
   with aspects (a), (b), (c), (g), (h), (i), (m) and (n) being preferred, with aspects in which n=1 or 2 being preferred and aspects in which n=1 being preferred.

Some specific, but non-limiting examples of useful C-terminal extensions are the following amino acid sequences: A, AA, AAA, G, GG, GGG, AG, GA, AAG, AGG, AGA, GGA, GAA or GAG.

In an embodiment of the invention, any C-terminal extension present in a Nav1.7 binder, Navβ binder, or Nav1.7-Navβ bispecific binder does not contain a free cysteine residue (unless said cysteine residue is used or intended for further functionalization, for example for PEGylation).

### Conjugates

The Nav1.7 binders, Navβ binders, or Nav1.7-Navβ bispecific binders disclosed herein may also be conjugated to a chemical moiety. Such conjugated binders are an embodiment of the present invention. The chemical moiety may be, inter alia, a polymer, a radionuclide or a cytotoxic factor. In particular embodiments, the chemical moiety is a polymer that increases the half-life of the Nav1.7 binder, Navβ binder, or Nav1.7-Navβ bispecific binder in the body of a subject. Suitable polymers include, but are not limited to, hydrophilic polymers, which include but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The Nav1.7 binders, Navβ binders, or Nav1.7-Navβ bispecific binders disclosed herein may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶ Fe.

The Nav1.7 binders may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The Nav1.7 binder, Navβ binder, or Nav1.7-Navβ bispecific binder may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating a Nav1.7 binder, Navβ binder, or Nav1.7-Navβ bispecific binder to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating binders are conventional and very well known in the art.

The present invention further provides nucleic acid molecules encoding any one of the Nav1.7 binders, Navβ binders, or Nav1.7-Navβ bispecific binders disclosed herein. In particular embodiments, the nucleic acid molecule encoding the Nav1.7 binder comprises a nucleotide sequence selected from the group of nucleotide sequences set forth in SEQ ID NO: 273-283. In particular embodiments, the nucleic acid molecule encoding the Nav1.7 binder comprises a nucleotide sequence selected from the group of nucleotide sequences set forth in SEQ ID NO: 284-421. In particular embodiments, the nucleic acid molecule encoding the Navβ binder comprises a nucleotide sequence selected from the group of nucleotide sequences set forth in SEQ ID NO: 456-461.

The following examples are intended to promote a further understanding of the present invention. The amino acid sequences for the Nav1.7 binder, Navβ binder, or Nav1.7-Navβ bispecific binders and nucleic acid sequences encoding the Nav1.7 binder, Navβ binder, or Nav1.7-Navβ bispecific binders that are disclosed in the following examples are provided in Table 56. Various embodiments of the aforementioned binders comprise an amino acid sequence set forth in Table 56.

### Example 1

Generation of Stable Recombinant huNav1.7α Cell Lines

Different stable CHO FlpIn (ThermoFisher Scientific, catalog #R758-07) or HEK FlpIn (ThermoFisher Scientific, catalog #R750-07) transgenic cell lines were generated according to the manufacturer's instructions. To this purpose, different Nav1.7α constructs (human or rhesus) were cloned into pcDNA5/FRT (ThermoFisher Scientific, catalog #V601020). The amino acid sequences for huNav1.7α, rhNav1.7α, huNav1.1α, huNav1.2α, huNav 1.3α, huNav1.4α, huNav1.5α, huNav1.6α, and huNav1.8α are set forth in SEQ ID NO: 1, SEQ ID NO: 2, SEQ ID NO: 23, SEQ ID NO: 24, SEQ ID NO: 25, SEQ ID NO: 26, SEQ ID NO: 27, and SEQ ID NO: 28, respectively. The generation of HEK293 cell lines stably expressing huNav1.7a with and without the human β subunit is detailed elsewhere (Schmalhofer et al. Mol Pharmacol 74:1476-1484, 2008). HEK cell lines expressing huNav1.1α, huNav1.2α, huNav1.3α, huNav1.4α, huNav1.5α, huNav1.6α, or huNav1.8α were constructed.

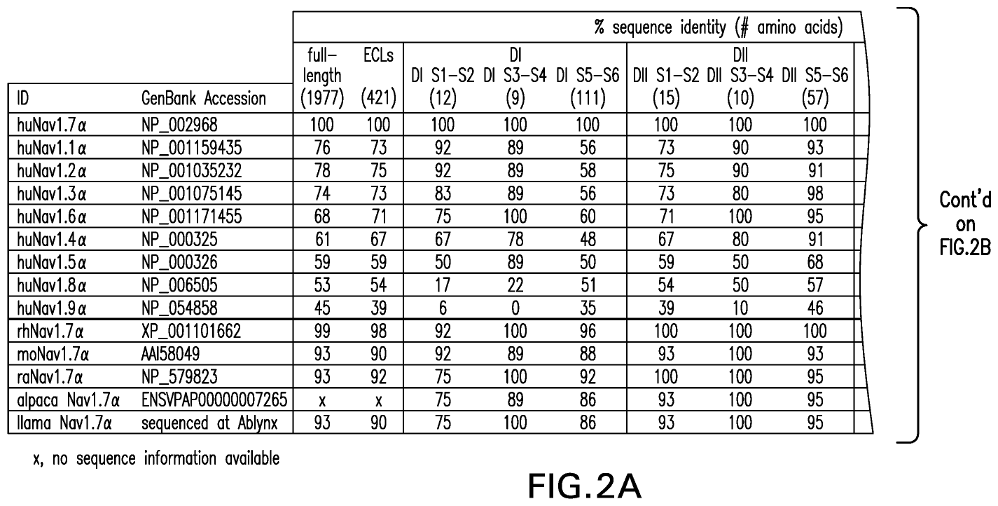

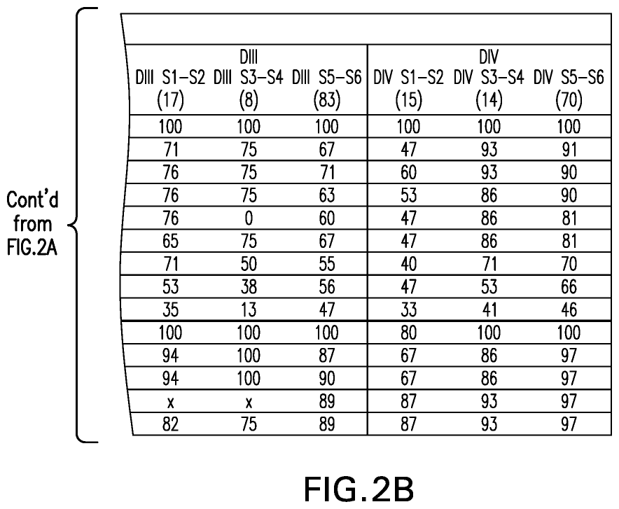

A detailed sequence comparison of the different extra-cellular loops (ECLs) of huNav1.7a to their ortholog and paralog counterparts is shown in FIGS. 2A-2B. Different splice variants of Nav1.7α exist that through interaction with β1 impact on the electrophysiological properties of the channel (Chatelier et al. 2008 J Neurophysiol 99: 2241; Farmer et al. 2012 PLoS ONE 7: e41750). The 5N11S variant of huNav1.7a (FIG. 32) was used consistently throughout the examples. The major technical drawbacks of Nav1.7α as a target for biologicals are its poor cell surface expression level combined with a limited accessibility to the extracellular surface.

For various experiments set forth in the examples, the Nav constructs where indicated were fused at the C-terminus via a P2A viral peptide linker (SEQ ID NO: 43) to a single polypeptide encoding sodium channel beta subunits β1 (SEQ ID NO: 40), β2 (SEQ ID NO: 41), and β3 (SEQ ID NO: 42) in tandem in which each β subunit is separated from the preceding β subunit by a P2A viral peptide linker (referred to herein as β1-β2-β3; See SEQ ID NO:21). The P2A peptide linker facilitates a co-translational cleavage event that effectively liberates polypeptides N-terminal and C-terminal to it.

### Plasmid Constructs and Expression Vectors

Table 7 gives an overview of all plasmid constructs and expression vectors.

TABLE 7
Overview plasmid DNA constructs

| Plasmid ID | Description |
|---|---|
| pCMV6-AC-Myc-DDK- | Origene clone with wild type huNav1.7α sequence |
| NM_002977.1 | (NM_002977.1) |
| pFRT/lacZEO | Basic vector for generation of Flp-In compatible cell |
| | backgrounds |
| pcDNA3.1-CO_huNav1.7 | Codon-optimized human source sequence |
| pcDNA3.1-CO_rhNav1.7 | Codon-optimized rhesus source sequence |
| pcDNA3.1- | Codon-optimized huNav1.7α/Nav1.5α chimera source |
| CO_huNav157chim14 | sequence (ECLs and transmembrane helices from Nav1.7α |
| | ICLs from Nav1.5α) \| combines native extracellular |
| | conformation of Nav1.7α with increased expression levels of |
| | Nav1.5α |
| pJTI-R4-DEST- | Codon-optimized huNav1.7α/Nav1.5α chimera and the |
| CO_huNav157chim14- | Navβ1-β3 subunits (β1-β2-β3) as picoRNA viral fusion source |
| PV_SCN1B-SCN2B-SCN3B | sequence |
| pJTI-R4-DEST- | Codon-optimized huNav1.7α and the Navβ1-β3 subunits (β1- |
| CO_huNav1.7-PV_SCN1B- | β2-β3) as picoRNA viral fusion source sequence |
| SCN2B-SCN3B | |
| pVAX1-NM_002977.1 | Vector for DNA immunizations with wild type huNav1.7α |
| | sequence |
| pcDNA3.1/Hygro- | Vector for cell line transfections with wild type huNav1.7α |
| NM_002977.1 | sequence |
| pcDNA5/FRT-NM_002977.1 | Vector for Flp-In cell line transfections with wild type |
| | huNav1.7α sequence |
| pcDNA5/FRT-CO_huNav1.7 | Vector for Flp-In cell line transfections with codon-optimized |
| | huNav1.7α sequence |
| pVAX1-CO_huNav1.7 | Vector for DNA immunizations with codon-optimized |
| | huNav1.7α sequence |
| pcDNA5/FRT- | Vector for Flp-In cell line transfections with huNav157 |
| huNav157chim14 | chimera 14 |
| pVAX1- | Vector for DNA immunizations with huNav157 chimera 14 |
| CO_huNav157chim14 | |
| pcDNA5/FRT- | Vector for Flp-In cell line transfections with huNav157 |
| CO_huNav157chim14- | chimera 14 and the Navβ1-β3 subunits (β1-β2-β3) |
| PV_SCN1B-SCN2B-SCN3B | |
| pcDNA5/FRT- | Vector for Flp-In cell line transfections with codon-optimized |
| CO_huNav1.7-PV_SCN1B- | huNav1.7α sequence and the Navβ1-β3 subunits (β1-β2-β3) |
| SCN2B-SCN3B | |
| pcDNA5/FRT-CO_huNav1.7 | Vector for Flp-In cell line transfections with codon-optimized |
| (P149_D150insFLAG) | huNav1.7α with triple FLAG tag inserted between aa 149 and |
| | 150 (S1 of Domain 1) for cell surface expression detection via |
| | tag |
| pcDNA5/FRT-CO_huNav1.7 | same as above but triple FLAG inserted between aa 148 and |
| (P148_P149insFLAG) | 149 |
| pcDNA3.1-CO_huNav1.5α | Codon-optimized human Nav1.5α source sequence |
| pcDNA5/FRT- | Vector for Flp-In cell line transfections with codon-optimized |
| CO_huNav1.5-PV_SCN1B- | huNav1.5α sequence and the Navβ1-β3 subunits (β1-β2-β3) to |
| SCN2B-SCN3B | be used as controls in selections and screening |
| pcDNA3.1- | Codon-optimized huNav1.7α/Nav1.5α chimera source |
| CO_huNav157chim1* | sequence (DI, DII and DIII from Nav1.7α, DIV from |
| | Nav1.5α) |
| pcDNA3.1- | Codon-optimized huNav1.7/Nav1.5α chimera source |
| CO_huNav157chim2* | sequence (DI, DII and DIV from Nav1.7α, DIII from |
| | Nav1.5α) |
| pcDNA3.1- | Codon-optimized huNav1.7α/Nav1.5α chimera source |
| CO_huNav157chim3* | sequence (DI, DIII and DIV from Nav1.7α, DII from |
| | Nav1.5α) |
| pcDNA3.1- | Codon-optimized huNav1.7α/Nav1.5α chimera source |
| CO_huNav157chim4* | sequence (DII, DIII and DIV from Nav1.7α, DI from |
| | Nav1.5α) |
| pcDNA3.1- | Codon-optimized huNav1.7α/Nav1.5α chimera source |
| CO_huNav157chim5* | sequence (DI, DII and DIII from Nav1.5α, DIV from |
| | Nav1.7α) |
| pcDNA3.1- | Codon-optimized huNav1.7α/Nav1.5α chimera source |
| CO_huNav157chim6* | sequence (DI, DII and DIV from Nav1.5α, DIII from |
| | Nav1.7α) |
| pcDNA3.1- | Codon-optimized huNav1.7α/Nav1.5α chimera source |
| CO_huNav157chim7* | sequence (DI, DIII and DIV from Nav1.5α, DII from |
| | Nav1.7α) |
| pcDNA3.1- | Codon-optimized huNav1.7α/Nav1.5α chimera source |
| CO_huNav157chim8* | sequence (DII, DIII and DIV from Nav1.5α, DI from |
| | Nav1.7α) |
| pcDNA3.1- | Codon-optimized huNav1.7α/Nav1.5α chimera source |
| CO_huNav157chim9* | sequence (DI, DII, DIII and DIV VSD from Nav1.7α, DIV |
| | S5-S6 from Nav1.5α) |
| pcDNA3.1- | Codon-optimized huNav1.7α/Nav1.5α chimera source |
| CO_huNav157chim10* | sequence (DI, DII, DIII VSD and DIV from Nav1.7α, DIII |
| | S5-S6 from Nav1.5α) |
| pcDNA3.1- | Codon-optimized huNav1.7α/Nav1.5α chimera source |
| CO_huNav157chim11* | sequence (DI, DI VSD, DIII and DIV from Nav1.7α, DII S5- |
| | S6 from Nav1.5α) |
| pcDNA3.1- | Codon-optimized huNav1.7α/Nav1.5α chimera source |
| CO_huNav157chim12* | sequence (DI VSD, DII, DIII and DIV from Nav1.7α, DI S5- |
| | S6 from Nav1.5α) |
| pcDNA3.1- | Codon-optimized huNav1.7α/Nav1.5α chimera source |
| CO_huNav157chim18* | sequence (DI, DII, DIII and DIV S5-S6 from Nav1.7α, DIV |
| | VSD from Nav1.5α) |
| pcDNA3.1- | Codon-optimized huNav1.7α/Nav1.5α chimera source |
| CO_huNav157chim22* | sequence (DI, DII, DIII and DIV S3-S6 from Nav1.7α, DIV |
| | S1-S2 from Nav1.5α) |
| pcDNA5/FRT-CO_rhNav1.7- | Vector for Flp-In cell line transfections with codon-optimized |
| PV_SCN1B-SCN2B-SCN3B | rhNav1.7α sequence and the human Navβ1-β3 subunits (β1- |
| | β2-β3) |
| pcDNA5/FRT- | Vector for Flp-In cell line transfections with codon-optimized |
| CO_huNav1.7(N146S, V194I, | huNav1.7α sequence containing all DI polymorphisms of |
| F276V, R277Q, E281V, V331M, | rhNav1.7α and the human Navβ1-β3 subunits (β1-β2-β3) |
| E504D, D507E, S508N, N533S)- | |
| PV_SCN1B-SCN2B-SCN3B | |
| pcDNA5/FRT-CO_rhNav1.7- | Vector for Flp-In cell line transfections with codon-optimized |
| PV_rhSCN1B-rhSCN2B- | rhNav1.7α sequence and the rhesus Navβ1-β3 subunits (β1- |
| rhSCN3B | β2-β3) |
| pcDNA5/FRT- | Vector for Flp-In cell line transfections with codon-optimized |
| CO_huNav1.7(F276V)- | huNav1.7α sequence containing extracellular DI rhNav1.7α |
| PV_SCN1B-SCN2B-SCN3B | polymorphism F276V and the human Navβ1-β3 subunits (β1- |
| | β2-β3) |
| pcDNA5/FRT- | Vector for Flp-In cell line transfections with codon-optimized |
| CO_huNav1.7(R277Q)- | huNav1.7α sequence containing extracellular DI rhNav1.7α |
| PV_SCN1B-SCN2B-SCN3B | polymorphism R277Q and the human Navβ1-β3 subunits (β1- |
| | β2-β3) |
| pcDNA5/FRT- | Vector for Flp-In cell line transfections with codon-optimized |
| CO_huNav1.7(E281V)- | huNav1.7α sequence containing extracellular DI rhNav1.7α |
| PV_SCN1B-SCN2B-SCN3B | polymorphism E281V and the human Navβ1-β3 subunits (β1- |
| | β2-β3) |
| pcDNA5/FRT- | Vector for Flp-In cell line transfections with codon-optimized |
| CO_huNav1.7(V331M)- | huNav1.7α sequence containing extracellular DI rhNav1.7α |
| PV_SCN1B-SCN2B-SCN3B | polymorphism V331M and the human Navβ1-β3 subunits |
| | (β1-β2-β3) |
| pcDNA5/FRT- | Vector for Flp-In cell line transfections with codon-optimized |
| CO_huNav1.7(Q1530P)- | huNav1.7α sequence containing extracellular DIV rhNav1.7α |
| PV_SCN1B-SCN2B-SCN3B | polymorphism Q1530P and the human Navβ1-β3 subunits |
| | (β1-β2-β3) |
| pcDNA5/FRT- | Vector for Flp-In cell line transfections with codon-optimized |
| CO_huNav1.7(H1531Y)- | huNav1.7α sequence containing extracellular DIV rhNav1.7α |
| PV_SCN1B-SCN2B-SCN3B | polymorphism H1531Y and the human Navβ1-β3 subunits |
| | (β1-β2-β3) |
| pcDNA5/FRT- | Vector for Flp-In cell line transfections with codon-optimized |
| CO_huNav1.7(E1534D)- | huNav1.7α sequence containing extracellular DIV rhNav1.7α |
| PV_SCN1B-SCN2B-SCN3B | polymorphism E1534D and the human Navβ1-β3 subunits |
| | (β1-β2-β3) |

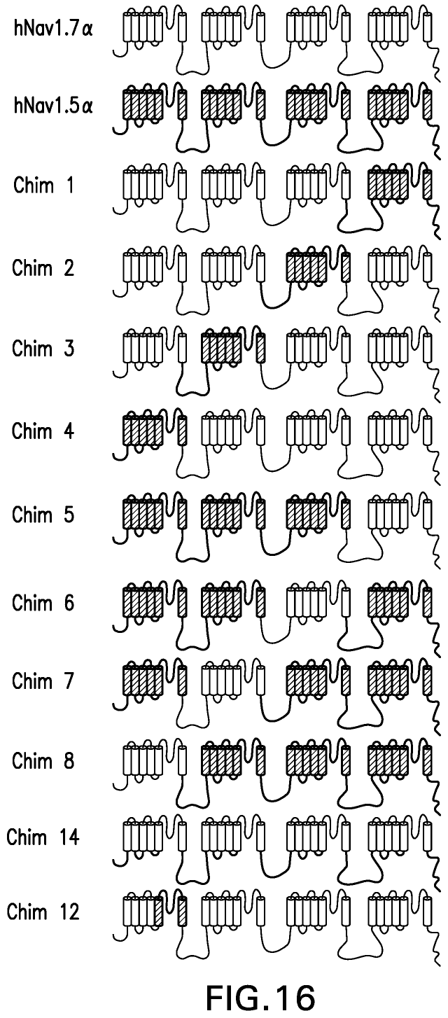

*huNav157 chimeras are schematically drawn in FIG. 16.

Generation of HEK293T Cells, Transiently Transfected with Different huNav1.7α Constructs

To this purpose, different Nav1.7α constructs were cloned into pcDNA3.1 (ThermoFisher Scientific, catalog #V79020) and plasmid DNA was prepared from Escherichia coli TOP10 cells. HEK293T cells were seeded at a concentration of 1.5×10⁶ per T75 flask and incubated overnight at 37° C. in DMEM (Dulbecco's modified Eagle's medium; Gibco, catalog #31966) supplemented with 10% FBS (fetal bovine serum, Sigma. Catalog #F7524). The medium was then replaced by Opti-MEM medium (Gibco, catalog #31985). A mixture of 9 μg plasmid DNA, 27 μL, Fugene 6 (Promega, catalog #E2691) in a final volume of 1 mL Opti-MEM was incubated for 15 min at room temperature and then added to the cells. After 3 hours incubation at 37° C., 10 mL of DMEM supplemented with 20% FBS was added and incubation continued. After 48 hours, cells were washed with phosphate buffered saline (PBS) and resuspended with 4 mL of trypsin EDTA (Gibco, catalog #25200-056) followed by addition of 6 mL DMEM medium supplemented with 10% FBS.

### Membrane Preparations

On Day 1, suspend pellet in 3 mL HB (250 mM Sucrose, 25 mM HEPES, pH 7.5)+μL Mammalian Protease Inhibitor cocktail+30 μL Benzonase/Nuclease-Dnase (25 U/μL) PER 1 billion cells; dounce homogenize with 5 strokes of a Type B/tight fit pestle (glass homogenizer); transfer homogenized cells to Nalgene 3119-0050 Oak Ridge centrifuge tubes and centrifuge at 5 k×g (6,025 rpm) for 30 minutes at 4° C. Collect supernatant fraction (and store on ice (pellet P1). Suspend pellet in 2 mL HB. Repeat dounce homogenization and transfer homogenized cells to fresh 50 mL falcon tubes. Increase the volume to 50 mL with HB. Centrifuge at 2 k xg (3,161 rpm) in for 15 minutes at 4° C.; collect the supernatant fraction, and pool with supernatant fraction collected above (P1). Suspend pellet in 2 mL HB. Repeat dounce homogenization. Increase volume to 50 mL with HB. Repeat 2K xg centrifugation. Collect the supernatant fraction and pool with the supernatant fraction collected above (P1). Transfer pooled supernatant fraction to fresh Nalgene tubes. Fill to fill line with HB. (P1) Suspend remaining pellet and transfer to fresh Nalgene tube. Fill to fill-line with HB to produce pellet 2 (P2). Centrifuge P1 & P2 at 39,800 xg (17 k rpm) for 45 minutes at 4° C. Keep 1 mL of supernatants (s1a+s2a). Store in −80° C. and decant remainder of supernatant fractions. Suspend pellets (P1+P2) in 0.1 M FB (100 mM NaCl, 25 mM Tris-HCl pH7.5). Repeat centrifugation at 39.8 k xg for 45 minutes at 4° C. Keep 1 mL of supernatants (s1b+s2b). Store at −80° C. Decant remainder of supernatants. Store pellets (P1+P2) on ice in 4° C. overnight.

On Day 2, suspend pellets in 1.5 M FB (1.5 M NaCl, 25 mM Tris-HCl pH7.5); dounce homogenize with 5 strokes of a Type B/tight fit pestle (glass homogenizer); transfer pellet to Nalgene 3119-0050 tube(s) and fill to fill line with 1.5 M FB; centrifuge at 39.8 k xg for 45 min at 4C; remove supernatant fraction and store pellets at −80° C. (SA).

Pool like pellets in 5-10 mL 1.5 M FB; dounce homogenize with 5 strokes of a Type B/tight fit pestle (glass homogenizer); return membrane to Nalgene tube and again fill to fill line with 1.5 M FB; repeat centrifuge at 39,800 xg (17 k rpm) for 45 minutes at 4° C. Remove supernatant fraction and store pellets at −80° C. (SB).

Suspend pellets in 5-10 mL 0.1 M FB; repeat dounce homogenization; return membrane to Nalgene tube and fill to fill line with 0.1 M FB; Centrifuge a 3rd time at 39,800 xg (17 k rpm) for 45 minutes at 4° C. Remove supernatant fraction and store pellet at −80° C. (SC).

Suspend pellets in 0.1 M FB; dounce homogenize with 5 strokes of a Type B/tight fit pestle (glass homogenizer); determine protein concentrations via Bradford assay; if desired, adjust concentration with 0.1 M FB; aliquot mem preparations, freeze on dry ice and store at 80° C.

### Binding FACS

Binding of the ISVDs to cell-expressed Nav1.7α was detected via murine anti-Flag (Sigma, catalog #F1804). Briefly, cells were resuspended in FACS buffer (PBS, 10% FBS, NaN₃) and transferred to a 96-well V-bottom plate at 1×10 5 cells/well. Purified FLAG3-tagged ISVD was diluted in FACS buffer and added to the cells for 30 minutes at 4° C. ISVD binding was detected by resuspending the samples subsequently in 100 μL murine anti-Flag at 1 μg/mL and 100 μL APC-labelled goat anti-mIgG (Jackson ImmunoResearch, catalog #115-135-164). Prior to the read-out, the samples were resuspended in 1 μg/mL propidium iodide (Sigma, catalog #P4170) to exclude dead cells. Between each step, the cells were centrifuged for 5 minutes at 200 grams and washed with 100 μL/well FACS buffer. An alternative approach used PE-labelled goat anti-murine IgG (Jackson ImmunoResearch, catalog #115-116-071) as detection antibody and 5 nM TOPRO3 (Molecular probes, catalog #T3605) as dead dye.

Control antibodies were detected as follows. Murine anti-Nav1.7α mAb S68-6 (Abcam, catalog #ab85015) was detected by PE-conjugated goat anti-murine IgG (Jackson ImmunoResearch, catalog #115-116-071) after fixation and permabilization of the cells with FIX & PERM kit according to the manufacturer's instructions (ThermoFisher Scientific, catalog #GAS003). Rabbit anti-Nav1.5α pAb (Alomone Labs, catalog #ASC-013) was detected with PE-conjugated goat anti-rabbit IgG (Jackson ImmunoResearch, catalog #711-116-152) after fixation and permabilization of the cells with FIX & PERM kit according to the manufacturer's instructions (ThermoFisher Scientific, catalog #GAS003). Rabbit anti-human 4 pAb (ThermoFisher Scientific, catalog #PAS-24142) was detected with PE-conjugated goat anti-rabbit IgG (Jackson ImmunoResearch, catalog #711-116-152).

### Immunizations

After approval of the Ethical Committee of the faculty of Veterinary Medicine (University Ghent, Belgium) or the Ethical Committee of the Ablynx Camelid Facility (LA1400575), 3 camelids were immunized with a CMV-promoter based DNA vector encoding codon optimized huNav1.7α, followed by codon optimized huNav157 chimera 14 DNA and membrane extracts prepared from recombinant HEK293 cells expressing huNav1.7α together with Navβ1, Navβ2 and Navβ3 (as described above).

### Cloning of Heavy Chain-Only Antibody Fragment Repertoires and Preparation of Phage

Following the final immunogen injection, blood samples were collected. From these blood samples, peripheral blood mononuclear cells (PBMCs) were prepared using Ficoll-Hypaque according to the manufacturer's instructions (Amersham Biosciences, Piscataway, NJ, US). From the PBMCs, total RNA was extracted and used as starting material for RT-PCR to amplify the VHH/ISVD-encoding DNA segments, essentially as described in WO05044858. Subsequently, phages were prepared according to standard protocols (see for example the prior art and applications filed by Ablynx N.V. cited herein) and stored after filter sterilization at 4° C. for further use.

### Selection of Nav1.7α Specific ISVDs Via Phage Display

VHH repertoires obtained from all camelids and cloned as phage library were subjected for two or three consecutive selection rounds to proteoliposome (PL) (5 μg/mL) or amphipol (amphipathic surfactant for maintaining solubilized membrane proteins in detergent-free solutions, catalog #A835, Anatrace) preparations (5 μg/mL) derived from HEK293 cells recombinantly expressing huNav1.7α together with Navβ1, Navβ2, and Navβ3 subunits (β1-β2-β3). Each selection round was performed in the presence of the following competing agents: 100 μg/mL of in house produced membrane extracts from HEK293 cells and 100 nM each of recombinant Navβ1 (Abnova, catalog #H00006324-P01), Navβ2 (Sino Biological, catalog #13859-H02H) and Navβ3 (Sino Biological, catalog #13500-H02H). After antigen incubation of the libraries and extensive washing; bound phage were eluted with trypsin (1 mg/mL) for 15 minutes and then the protease activity was immediately neutralized by applying 0.8 mM protease inhibitor ABSF. As a control, selections with in-house produced membrane extracts from HEK293 cells or without antigen were performed in parallel. Phage outputs were used to infect E. coli TG1 for analysis of individual VHH clones. Periplasmic extracts were prepared according to standard protocols (see for example WO03035694, WO04041865, WO04041863, WO04062551).

### Generation of ISVD Expression Constructs

Sequence analysis of ISVDs from phage display selection outputs was done according to commonly known procedures (Pardon et al., Nat Protoc 9: 674 (2014)). ISVD-containing DNA fragments, obtained by PCR with specific combinations of forward FR1 and reverse FR4 primers each carrying a unique restriction site, were digested with the appropriate restriction enzymes and ligated into the matching cloning cassettes of ISVD expression vectors (described below). The ligation mixtures were then transformed to electrocompetent Escherichia coli TG1 (60502, Lucigen, Middleton, WI) cells which were then grown under the appropriate antibiotic selection pressure. Resistant clones were verified by Sanger sequencing of plasmid DNA (LGC Genomics, Berlin, Germany). Monovalent ISVDs were expressed in E. coli TG1 from a plasmid expression vector containing the lac promoter, a resistance gene for kanamycin, an E. coli replication origin and an ISVD cloning site preceded by the coding sequence for the OmpA signal peptide. In frame with the ISVD coding sequence, the vector codes for a C-terminal FLAG3 (or CMYC3) and HIS6 tag. The signal peptide directs the expressed ISVDs to the periplasmic compartment of the bacterial host.

Unless specified otherwise, the tested clones herein comprise the ISVD amino acid sequence shown for it in Table 56 further fused at the C-terminus to a FLAG-HIS6 polypeptide (SEQ ID NO: 56) or HIS6. The amino acid positions in the ISVDs disclosed herein are numbered according to the Kabat numbering scheme.

### Generic Expression and Purification of ISVDs

E. coli TG-1 cells containing the ISVD constructs of interest were grown for 2 hours at 37° C. followed by 29 hours at 30° C. in baffled shaker flasks containing “5052” auto-induction medium (0.5% glycerol, 0.05% glucose, 0.2% lactose+3 mM MgSO₄). Overnight frozen cell pellets from E. coli expression cultures are then dissolved in PBS (1/12.5^{th} of the original culture volume) and incubated at 4° C. for one hour while gently rotating. Finally, the cells were pelleted down once more, and the supernatant containing the proteins secreted into the periplasmic space was stored for further purification. HIS6-tagged ISVDs were purified by immobilized metal affinity chromatography (IMAC) on either Ni-Excel (GE Healthcare) or Ni-IDA/NTA (Genscript) resins with Imidazole (for the former) or acidic elution (for the latter) followed by a desalting step (PD columns with Sephadex G25 resin, GE Healthcare) and if necessary, gel filtration chromatography (Superdex column, GE Healthcare) in PBS.

### Example 2

Selective Binding to huNav1.7α.

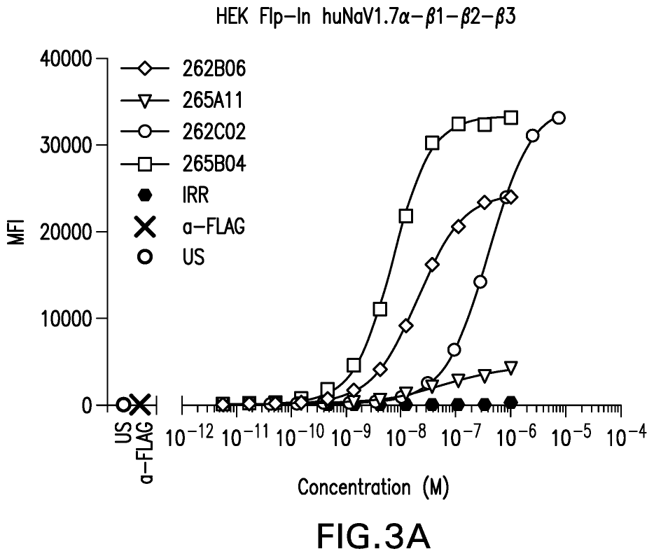

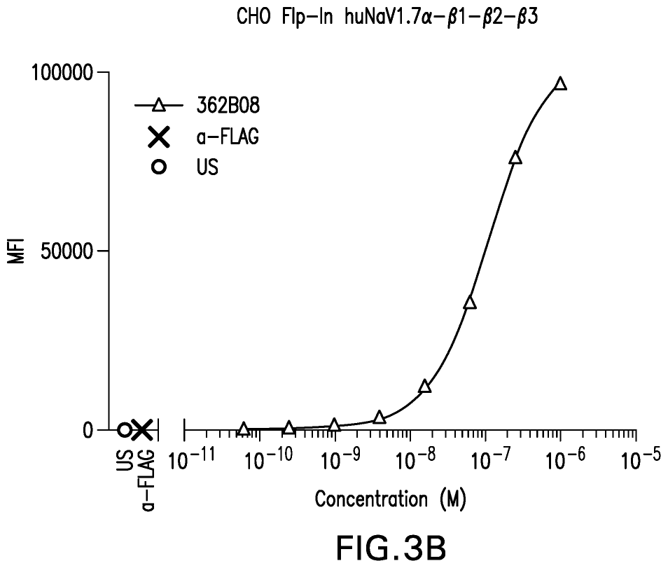

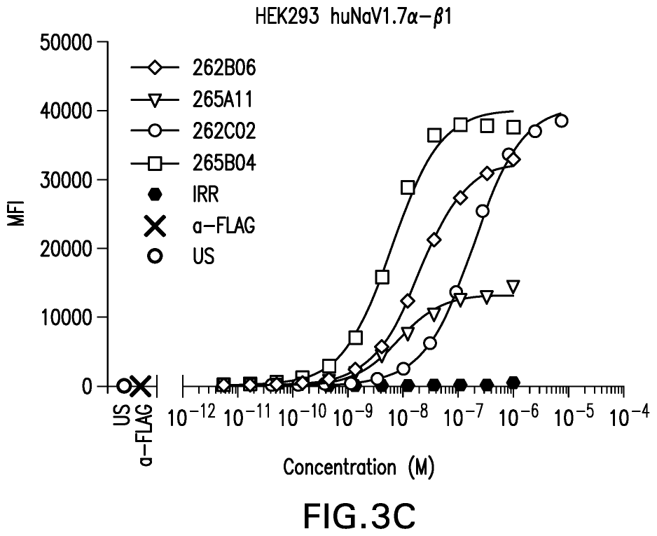

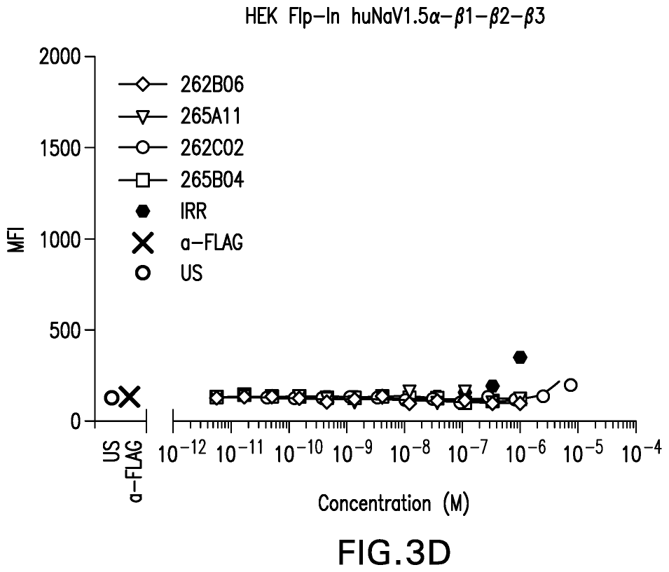

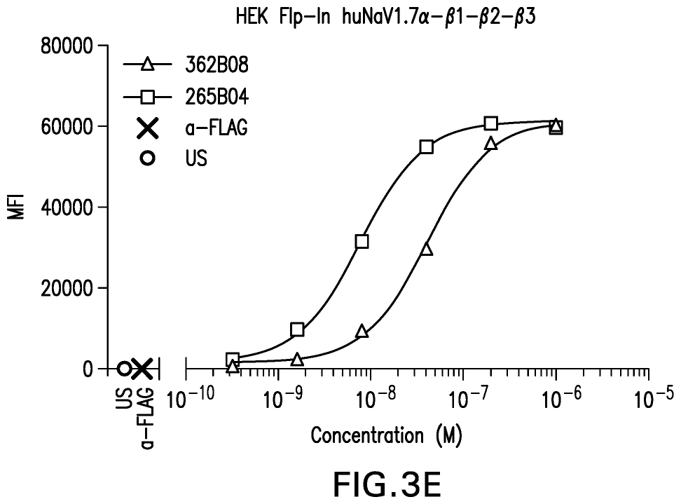

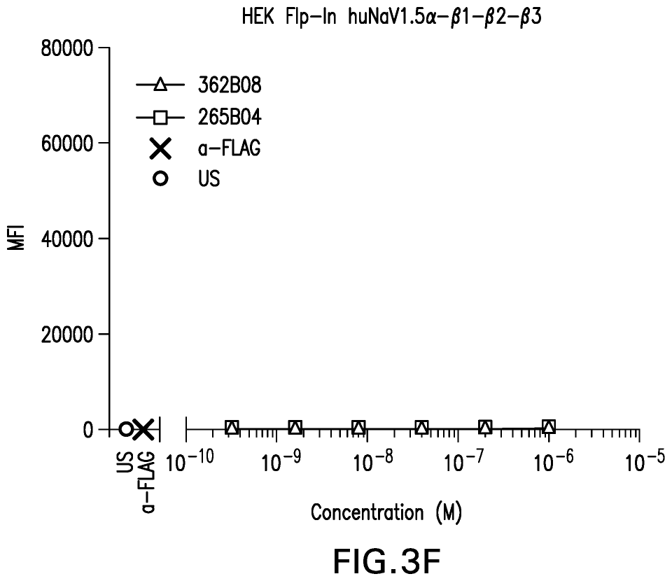

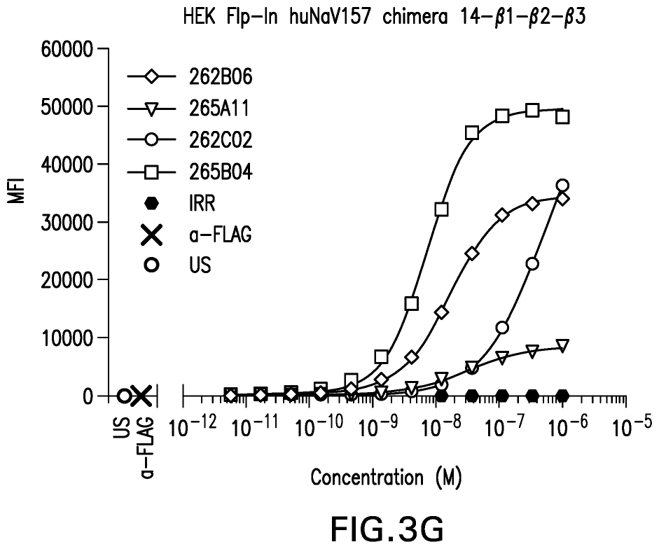

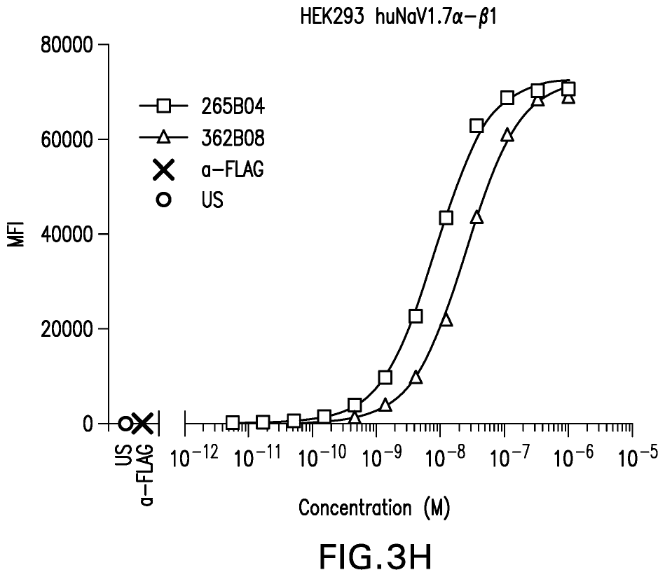

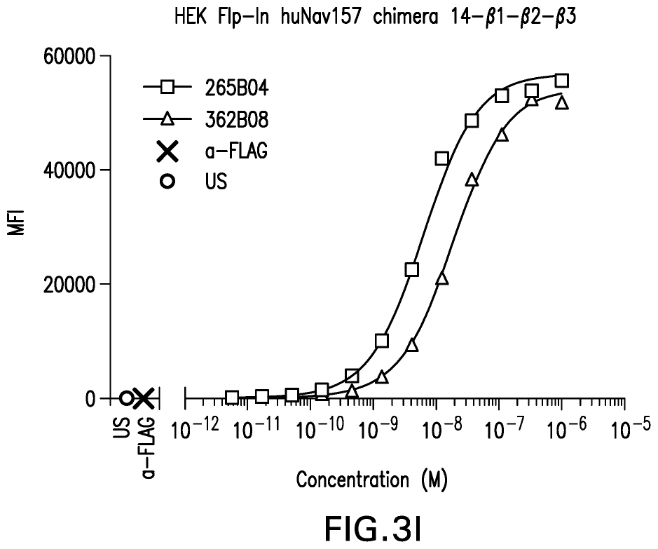

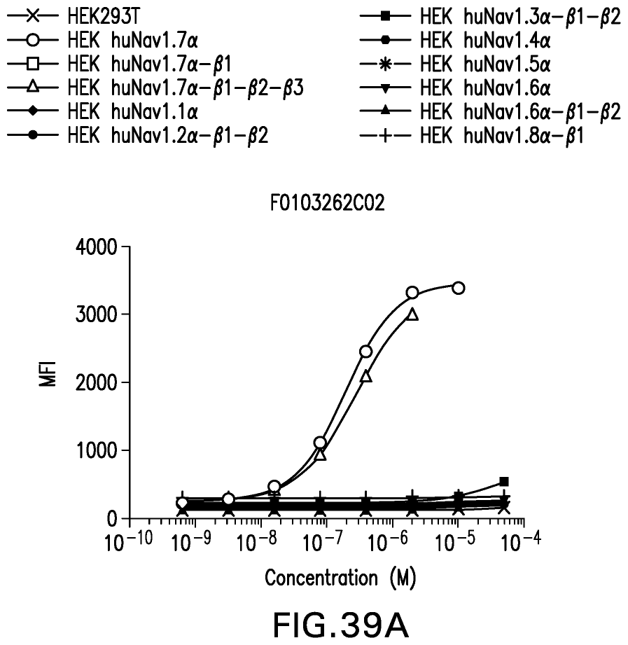

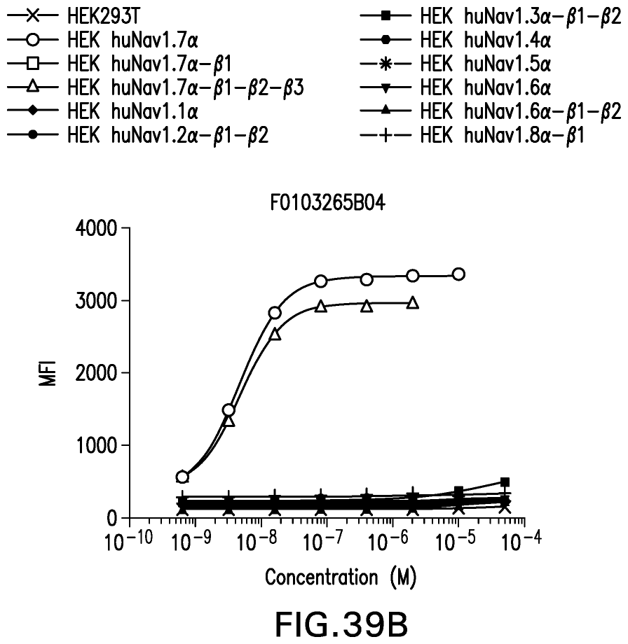

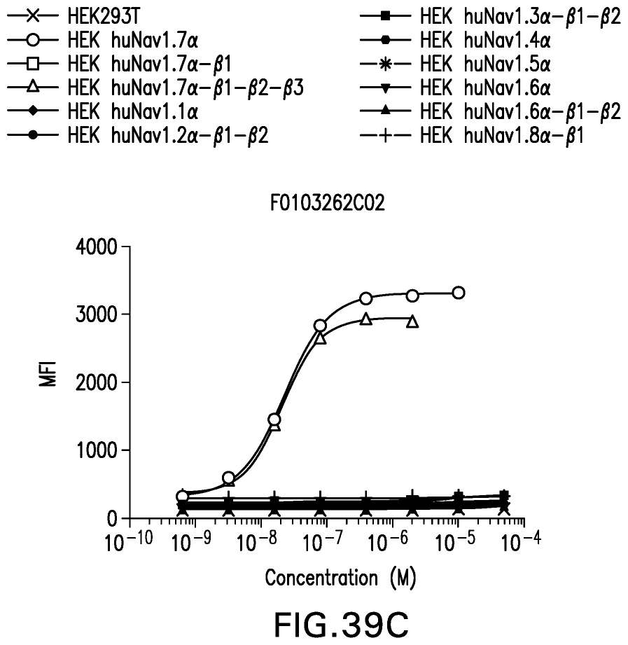

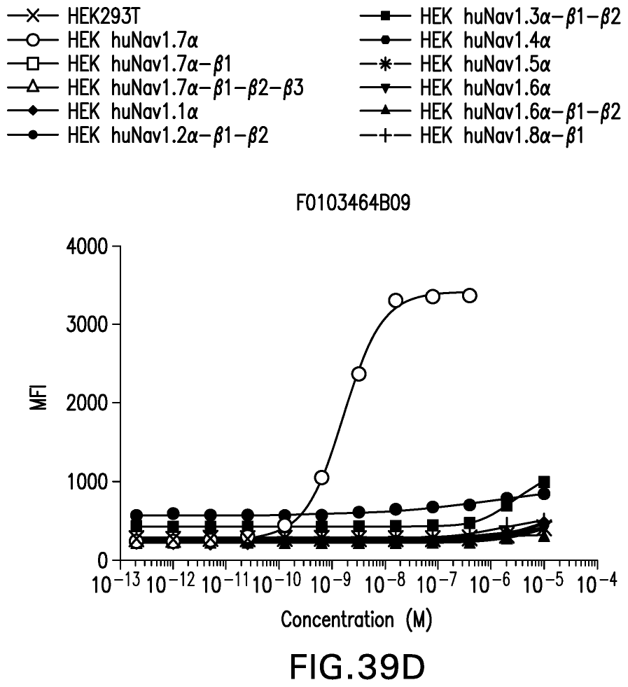

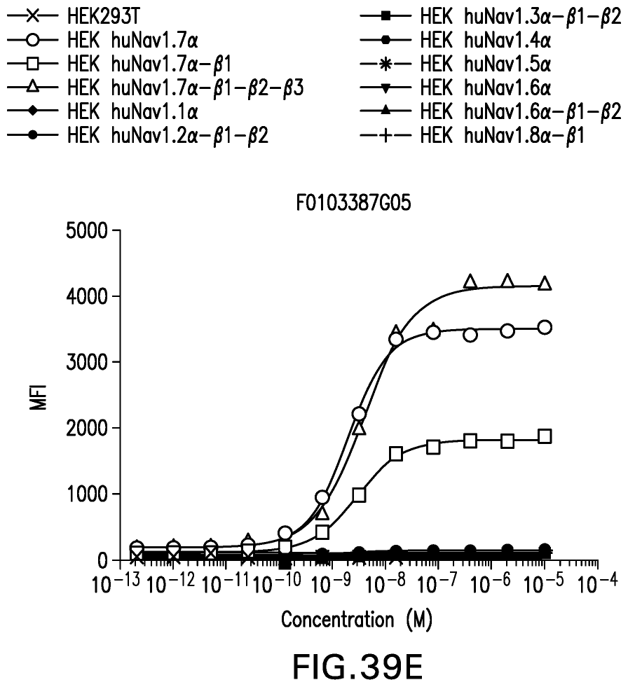

Crude periplasmic extracts containing ISVDs from phage display selections (as described above) were screened in FACS for binding to huNav1.7α but not to huNav1.5a. Confirmatory binding FACS experiments with purified FLAG3-HIS6 tagged ISVD proteins revealed that the ISVDs all bind selectively to different stable cell lines expressing huNav1.7α and huNav157 chimera 14 (extracellular and transmembrane sequences of huNav1.7α, combined with intracellular sequences of huNav1.7α and huNav1.8α and the Navβ1, Navβ2, and Navβ3 subunits (see Table 8; FIG. 3A-FIG. 3I)), but not to cell lines expressing rhNav1.7α, huNav1.1α, huNav1.2α, huNav1.3α, huNav1.4α, huNav1.5α, huNav1.6α or huNav1.8α. For example, FIGS. 39A-39E show that F0103262CO2, F0103265B04, F0103275B05, F0103464B09, and F0103387G05 are specific for huNav1.7α with no binding to huNav1.1α, huNav1.2α, huNav1.3α, huNav1.4α, huNav1.5α, huNav1.6α or huNav1.8α. As used in Table 8, the drawings, and throughout the description, Navβ1, Navβ2, and Navβ3 are human homologs unless specifically identified otherwise.

TABLE 8

| | | | | HEK | | | |
|---|---|---|---|---|---|---|---|
| | | CHO | HEK | FlpIn | | HEK | CHO |
| | | FlpIn | FlpIn | huNav | HEK293 | FlpIn | FlpIn |
| | | huNav1.7α + | huNav1.7α + | 157chimera14 + | huNav1.7α + | huNav1.5α + | rhNav1.7α + |
| | SEQ | β1-β2- β3 | β1-β2- β3 | β1- β2-β3 | β1 | β1-β2- β3 | β1-β2- β3 |
| | ID | (SEQ ID NO: 3) | (SEQ ID NO: 3) | (SEQ ID NO: 20) | (SEQ ID NO: 44) | (SEQ ID NO: 22) | (SEQ ID NO: 3) |
| ID # | NO: | pEC50 [M] | pEC50 [M] | pEC50 [M] | pEC50 [M] | pEC50 [M] | pEC50 [M] |
| F0103262B06 | 30 | 1.9E−08 | 2.5E−08 | 1.6E−08 | 2.0E−08 | — | — |
| F0103262C02 | 31 | 3.2E−07 | 3.8E−07 | 4.7E−07 | 1.8E−07 | — | — |
| F0103265A11 | 32 | 6.6E−08 | 3.7E−08 | 3.4E−08 | 8.4E−09 | — | — |
| F0103265B04 | 33 | 5.4E−09 | 8.1E−09 | 8.5E−09 | 5.5E−09 | — | — |
| F0103275B05 | 34 | 2.7E−08 | 3.7E−08 | 3.9E−08 | 2.8E−08 | — | — |
| F0103362B08 | 35 | 1.1E−07 | 4.2E−08 | ND | 5.9E−08 | — | — |
| F0103387G04 | 36 | 1.9E−08 | 1.6E−08 | ND | 6.7E−09 | — | 2.1E−07 |
| F0103387G05 | 37 | ND | 3.1E−09 | 3.6E−09 | 1.2E−09 | — | — |
| F0103345D07 | 38 | 2.2E−08 | ND | ND | 1.2E−08 | — | — |
| F0103464B09 | 39 | 4.1E−09 | ND | ND | 4.1E−09 | — | 2.7E−08 |

Mean pEC50 ± standard deviation;
—, no binding observed;
ND, not determined

The amino acid sequences for the ten ISVDs (Nav1.7 binders) without the FLAG-HIS6 peptide (SEQ ID NO: 56) are shown in SEQ ID NO: 46, 47, 48, 49, 50, 51, 52, 53, 54, and 55, respectively.

### Example 3

Affinity maturation was used to further improve the functional potencies of selected ISVDs by means of in vitro affinity maturation. In addition, as none of the selected ISVDs is cross-reactive to rhNav1.7α (with the exception of the weakly cross-reactive ISVD F0103387G04), the same process was applied to improve the NHP cross-reactivity to enable in vivo proof of concept (POC) studies in rhesus monkeys. In vitro affinity maturation of ISVDs is a two-stage process that aims to improve binding-related properties like affinity, species cross-reactivity or potency. First, all CDR-based residues are systematically changed to every possible amino acid on a one-by-one basis. The resulting libraries of single site substitution variants pooled per CDR are then screened for improvement of the desired property after which the hits are identified by means of Sanger sequencing. The beneficial single site substitutions are then combined into a library of combinatorial variants which are evaluated for further improvement of the desired property, followed by Sanger sequencing of hits. The generation the DNA fragments encoding the ISVD variants is either outsourced to commercial providers GeneWiz (South Plainfield, NJ) or IDT (Coralville, IA) or performed in house using commonly known molecular biology techniques such as site-directed mutagenesis, overlap extension PCR and oligonucleotide gene assembly (In Vitro Mutagenesis Protocols, 2^{nd} Edition (2002), Jeff Braman ed., Humana Press, Totowa NJ).

### Affinity Maturation of F0103275B05 & F0103387G04

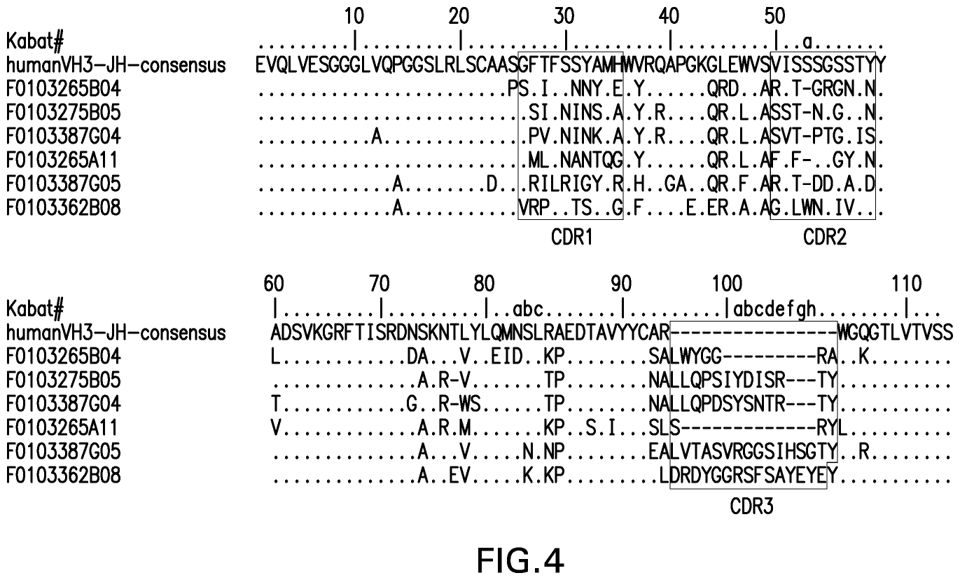

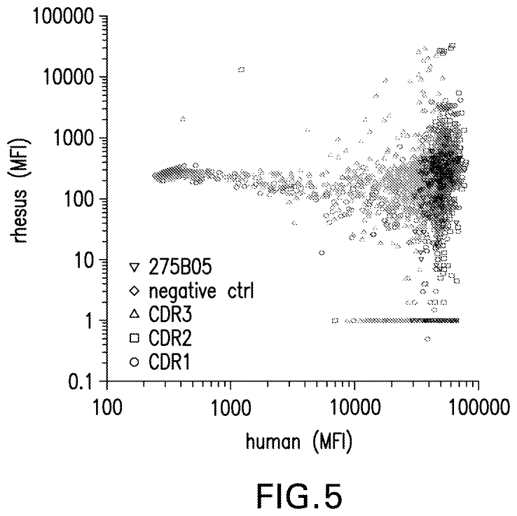

As ISVD F0103275B05 and rhNav1.7α cross-reactive F0103387G04 appear to be related ISVDs with highly similar CDRs (FIG. 4), it was decided to pursue these two ISVDs for affinity maturation in one and the same effort. A pooled single site saturation stage I library of F0103275B05 was constructed and crude periplasmic extracts of 2100 individual clones were prepared and screened in binding FACS to huNav1.7α and rhNav1.7α. Clones with a single mutation in CDR3, CDR2 or CDR1 residues showed an improved binding to rhNav1.7α, but much less so to huNav1.7α (FIG. 5).

The sequence analysis of 384 hits is summarized in Table 9. The stage I hits have substitutions in 7/10, 7/9, and 5/15 positions of respectively CDR1, CDR3 and CDR3. Interestingly, the substitutions in three of these positions (27, 28 and 53) recapitulate some of the differences between F0103275B05 and its rhNav1.7α cross-reactive relative F0103387G04 and thus bring additional confidence in the outcome of the stage I screening. These three substitutions were included in the design of the stage II combinatorial library (bottom row of Table 8), in which 11 positions were allowed to vary between the parental F0103275B05 and the highest ranked stage I hit residue. The stage II library thus captures 2¹¹=2048 different combinatorial variants.

TABLE 9
Summary sequence analysis of F0103275B05/F0103387G04 screening
stage I & design stage II affinity maturation libraries

| | | CDR1 | | | | | | | | | | CDR3 | | | | | | | | |
|---|---|---|---|---|---|---|---|---|---|---|---|---|---|---|---|---|---|---|---|---|
| Kabat # | | 26 | 27 | 28 | 29 | 30 | 31 | 32 | 33 | 34 | 35 | 50 | 51 | 52 | 53 | 54 | 55 | 56 | 57 | 58 |
| ′275B05 | | G | S | I | F | N | I | N | S | M | A | S | S | T | N | G | G | S | T | N |
| ′387G04 | | G | P | V | F | N | I | N | K | M | A | S | V | T | P | T | G | S | I | S |
| Rank | 1 | | P | V | | L | W | S | R | | R | Y | | | P | R | W | D | H | R |
| hits | 2 | | | | | W | | | W | | L | R | | | | | | R | W | |
| stage I | 3 | | | | | | | | V | | | V | | | | | | | | |
| | 4 | | | | | | | | Q | | | | | | | | | | | |

| | | | | | | | | | | | | | | | | | | | |
|---|---|---|---|---|---|---|---|---|---|---|---|---|---|---|---|---|---|---|---|
| Stage II design | G | P | V | F | NL | IW | N | SR | M | AR | SY | S | T | P | G | GW | SD | TW | N |

| | CDR3 | | | | | | | | | | | | | | |
|---|---|---|---|---|---|---|---|---|---|---|---|---|---|---|---|
| Kabat # | 93 | 94 | 95 | 96 | 97 | 98 | 99 | 100 | 100a | 100b | 100c | 100d | 100e | 101 | 102 |

| ′275B05 | | N | A | L | L | Q | P | S | I | Y | D | I | S | R | T | Y |
|---|---|---|---|---|---|---|---|---|---|---|---|---|---|---|---|---|
| ′387G04 | | N | A | L | L | Q | P | D | S | Y | S | N | T | R | T | Y |
| Rank | 1 | W | W | | | | T | | | | | | | | I | |
| hits | 2 | D | W | E | | | | | | | | | | | K | |
| stage I | 3 | | G | | | | | | | | | | | | F | |
| | 4 | | | | | | | | | | | | | | | |

| | | | | | | | | | | | | | | | |
|---|---|---|---|---|---|---|---|---|---|---|---|---|---|---|---|
| Stage II design | NR | AW | L | L | Q | P | S | I | T | D | I | S | R | TI | Y |

′274B05 = F0103275B05;
′387G04 = F0103387G04

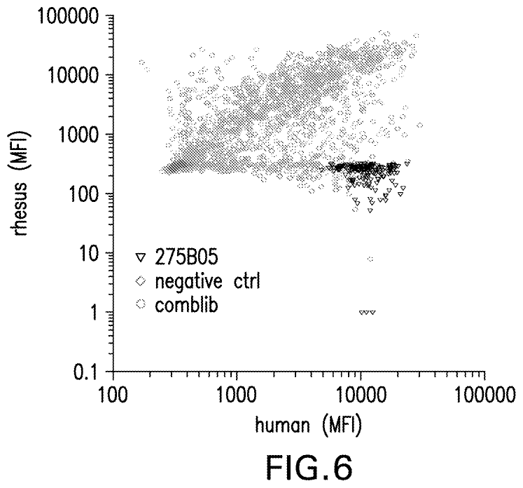

Crude periplasmic of 2100 clones of the stage II combinatorial library were prepared and screened in binding FACS on huNav1.7α and rhNav1.7α. A large fraction of the variants displayed improved binding to rhNav1.7α compared to the huNav1.7α-selective parental F0103275B05 (FIG. 6), indicating that the library design successfully captured and improved the promise of the stage I library. No improvements for binding to huNav1.7α were observed for stage II, in line with the observations during stage I. The sequence analysis of 300 hits is summarized in Table 10. Compared to a randomly picked reference sample, the top 25% of the hits are enriched for the N93R substitution but display a lower proportion of the N30L, I31W, A35R, G55W and T57W substitutions. Compared to a randomly picked reference sample, the bottom 25% of the hits displayed a lower proportion of the I31W and A35R substitutions. An analysis of the subset of the top 25% hits that did not carry the N93R mutation revealed that these were enriched for the S33R, S50Y and S56D substitutions and had a lower proportion of A94W, compared to the reference sample.

TABLE 10
Summary sequence analysis of F0103275B05 screening stage II affinity maturation libraries

| | CDR1 | | | | CDR2 | | | | CDR3 | | |
|---|---|---|---|---|---|---|---|---|---|---|---|
| Kabat # | N30L | I31W | S33R | A35R | S50Y | G55W | S56D | T57W | N93R | A94W | T101I |
| Reference | 47% | 44% | 51% | 39% | 40% | 43% | 47% | 47% | 42% | 53% | 56% |
| sample | | | | | | | | | | | |
| Top 25% of | 26% | 16% | 40% | 1% | 53% | 11% | 46% | 24% | 77% | 44% | 47% |
| hits | | | | | | | | | | | |
| Bottom 25% | 43% | 25% | 46% | 10% | 51% | 42% | 48% | 54% | 46% | 48% | 42% |
| of hits | | | | | | | | | | | |
| Top 25% of | 31% | 6% | 88% | 6% | 63% | 19% | 56% | 38% | NA | 19% | 50% |
| hits with N93 | | | | | | | | | | | |

NA, not applicable

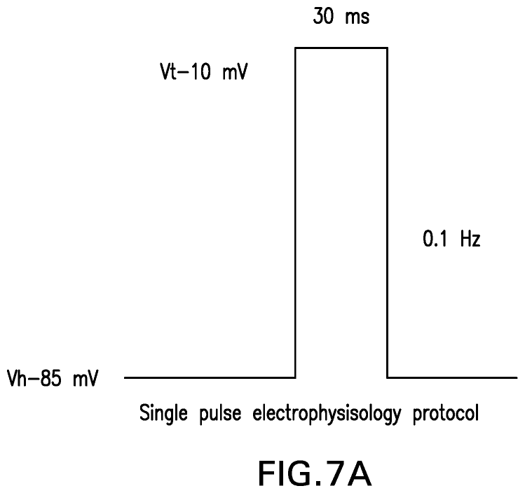

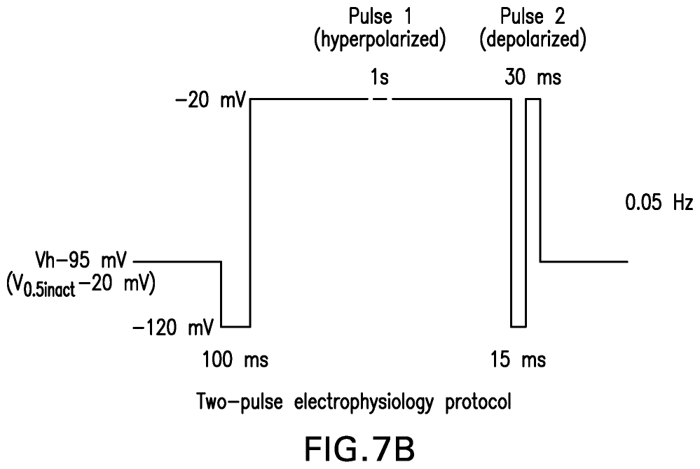

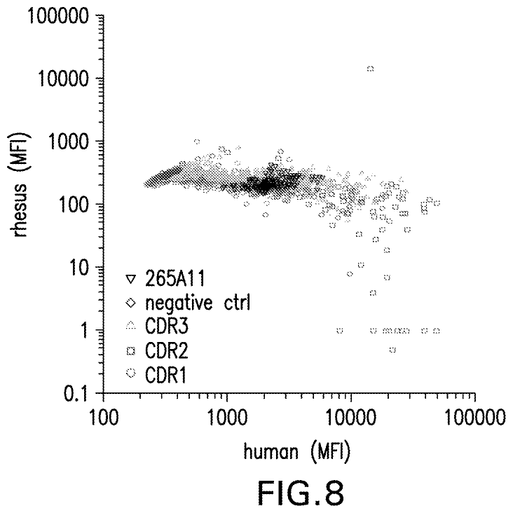

A number of combinatorial affinity maturation variants of F0103275B5 were then characterized in detail in binding FACS and electrophysiology (Table 11). All variants bound rhNav1.7α, many with greater affinity than F01033387G04. This was confirmed for most of them in 2-pulse (FIG. 7B) and single pulse (FIG. 7A) electrophysiology experiments. A subset of variants is equipotent on huNav1.7α and rhNav1.7α, with binding EC₅₀ values of ±20 nM. The minimal number of mutations to a achieve this is four (S33R, S50Y, S56D and N93R) as exemplified by F010301461. F0103387G04 remains the best binder to huNav1.7α, most likely due to differences compared to F0103275B05 in other CDR positions. Variant F010300659 was the first variant with good rhNav1.7α cross-reactivity to be characterized, and as such was selected for in vivo assessment.

TABLE 11
Summary binding and functional characterization of F01033275B05 affinity variants
Part 1

| | Kabat # (mutations vs. F01033275B05) | | | | | | | | | | | |
|---|---|---|---|---|---|---|---|---|---|---|---|---|
| ID # | S27 | I28 | I31 | S33 | S50 | N53 | G55 | S56 | T57 | N93 | A94 | T101 |
| F010300948 | P | V | . | R | . | P | . | . | . | R | W | I |
| F010301462 | P | V | . | R | Y | P | . | D | . | R | . | . |
| F010301459 | P | V | . | R | . | P | . | D | . | R | . | . |
| F010301461 | . | . | . | R | Y | . | . | D | . | R | . | . |
| F010300900 | P | V | . | R | . | P | . | D | . | R | W | I |
| F010300880 | P | V | . | R | . | P | . | D | . | R | W | . |
| F010301460 | P | V | . | R | Y | P | . | . | . | R | . | . |
| F010300990 | P | V | . | R | Y | P | . | . | . | . | . | I |
| F010301000 | P | V | . | R | Y | P | . | D | . | . | . | I |
| F010300468 | . | . | . | . | . | . | . | . | . | R | . | . |
| F010300796 | P | V | . | R | Y | P | . | D | . | . | . | . |
| F010300631 | P | V | . | . | Y | P | . | . | . | R | . | . |
| F010300684 | P | V | W | . | . | P | . | . | . | R | . | I |
| F010300659 | P | V | . | . | Y | P | W | D | W | R | W | . |
| F0103387G04 | P | V | . | . | . | P | . | . | . | . | . | . |
| F010300477 | . | . | . | . | . | . | . | . | . | . | W | . |
| F010300316 | . | . | . | . | . | . | . | . | W | . | . | . |
| F0103275B05 | . | . | . | . | . | . | . | . | . | . | . | . |

Part 2

| | | | | HEK | |
|---|---|---|---|---|---|
| | CHO FlpIn | HEKa/β1 | | rhNav1.7α + β1- | |
| | rhNav1.7α + β1- | (SEQ ID NO: 40) | HEKa | β2-β3 | HEKa/β1 |
| | β2-β3 | Nav1.7 | huNav1.70α | (SEQ ID NO: 4) | (SEQ ID NO: 40) |
| | (SEQ ID NO: 4) | (SEQ ID NO: 1) | (SEQ ID NO: 1) | single pulse | single pulse |
| ID # | EC50 [M] | EC50 [M] | EC50 [M] | IC50 [M] | IC50 [M] |
| F010300948 | 2.0E−08 | 6.8E−08 | 6.9E−08 | 5.8E−08 | 3.2E−07 |
| F010301462 | 2.3E−08 | 2.2E−08 | 3.5E−08 | ND | ND |
| F010301459 | 2.3E−08 | 1.8E−08 | 2.2E−08 | ND | ND |
| F010301461 | 2.4E−08 | 2.1E−08 | 2.8E−08 | ND | ND |
| F010300900 | 2.5E−08 | 7.0E−08 | 2.0E−08 | 3.6E−08 | 2.0E−07 |
| F010300880 | 2.6E−08 | 4.4E−08 | 1.5E−08 | 1.2E−07 | 2.7E−08 |
| F010301460 | 3.2E−08 | 2.6E−08 | 1.5E−08 | ND | ND |
| F010300990 | 3.8E−08 | 6.5E−08 | 4.3E−09 | 1.0E−07 | 2.5E−07 |
| F010301000 | 4.0E−08 | 6.0E−08 | 8.5E−09 | ND | ND |
| F010300468 | 4.0E−08 | ND | ND | ND | ND |
| F010300796 | 4.4E−08 | 3.8E−08 | 5.8E−09 | ND | ND |
| F010300631 | 4.5E−08 | ND | ND | 1.7E−07 | 7.3E−08 |
| F010300684 | 5.0E−08 | ND | ND | 2.1E−07 | 2.0E−07 |
| F010300659 | 5.5E−08 | 2.7E−08 | 9.7E−09 | 1.8E−07 | 8.5E−08 |
| F0103387G04 | 1.5E−07 | 6.7E−09 | 3.2E−09 | ND | ND |
| F010300477 | 1.7E−07 | ND | ND | 1.2E−06 | 9.6E−08 |
| F010300316 | 1.7E−07 | ND | ND | ND | ND |
| F0103275B05 | — | 4.8E−08 | 1.0E−08 | ND | ND |

Part 3

| | |
|---|---|
| huNav1.7α | rhNav1.7α |
| (SEQ ID NO: 1) | (SEQ ID NO: 2) |
| 2-pulse IC50 [M] | 2-pulse IC50 [M] |

| ID # | P1 | P2 | P1 | P2 |
|---|---|---|---|---|
| F010300948 | ND | ND | ND | ND |
| F010301462 | ND | ND | ND | ND |
| F010301459 | ND | ND | ND | ND |
| F010301461 | ND | ND | ND | ND |
| F010300900 | ND | ND | ND | ND |
| F010300880 | ND | ND | ND | ND |
| F010301460 | ND | ND | ND | ND |
| F010300990 | ND | ND | ND | ND |
| F010301000 | ND | ND | ND | ND |
| F010300468 | 5.0E−06 | 4.0E−06 | 6.0E−06 | 3.0E−06 |
| F010300796 | ND | ND | ND | ND |
| F010300631 | 1.0E−06 | 8.0E−07 | 2.0E−06 | 8.0E−07 |
| F010300684 | 2.0E−06 | 2.0E−06 | 7.0E−06 | 1.0E−06 |
| F010300659 | 1.0E−06 | 8.0E−07 | 1.0E−06 | 7.0E−07 |
| F0103387G04 | 7.0E−07 | 4.0E−07 | 7.0E−06 | 3.0E−06 |
| F010300477 | 2.0E−06 | 2.0E−06 | 3.0E−06 | 1.0E−06 |
| F010300316 | 2.0E−06 | 1.0E−06 | 5.0E−06 | 4.0E−06 |
| F0103275B05 | ND | ND | ND | ND |

—, no binding detected; ND, not determined

### Affinity Maturation of F01033265A11

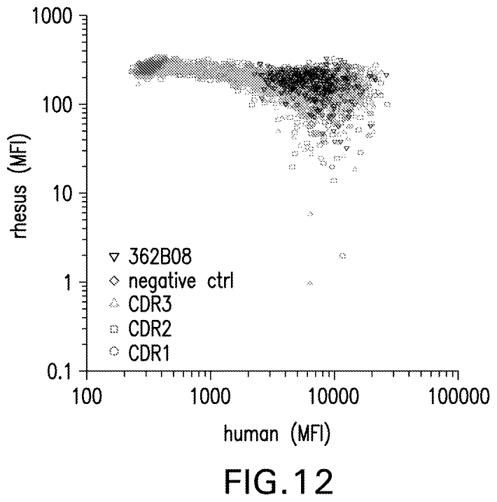

A pooled single site saturation library of F0103265A11 was constructed and crude periplasmic extracts of 1848 individual clones were prepared and screened in binding FACS on huNav1.7a and rhNav1.7α. Clones with a single mutation in CDR2, CDR3 or CDR1 residues showed an improved binding to huNav1.7α, but not to rhNav1.7α (FIG. 12).

The sequence analysis of 288 hits is summarized in Table 12. The stage I hits have substitutions in 3 of 10, 7 of 11, and 4 of 6 positions of respectively CDR1, CDR3 and CDR3. Of interest, four CDR2 positions (51, 53, 56 and 57) have substitutions to a Trp residue. The stage II library design captures 2 11=2048 different combinatorial variants.

TABLE 12
Summary sequence analysis of F0103265A11 screening stage
I & design stage II affinity maturation libraries

| | | CDR1 | | | | | | | | | | CDR2 | | | | |
|---|---|---|---|---|---|---|---|---|---|---|---|---|---|---|---|---|
| Kabat # | | 26 | 27 | 28 | 29 | 30 | 31 | 32 | 33 | 34 | 35 | 50 | 51 | 52 | 53 | 54 |
| F0103265A11 | | G | M | L | F | N | A | N | T | Q | G | F | I | F | S | G |
| Rank | 1 | | | K | | Y | R | | | | | | W | | W | |
| hits | 2 | | | | | L | | | | | | | | | | |
| stage I | 3 | | | | | R | | | | | | | | | | |
| | 4 | | | | | H | | | | | | | | | | |
| | 5 | | | | | F | | | | | | | | | | |

| | | | | | | | | | | | | | | | |
|---|---|---|---|---|---|---|---|---|---|---|---|---|---|---|---|
| Stage II design | G | M | L | F | NY | AR | N | T | Q | G | F | IW | F | SW | G |

| | | CDR2 | | | | | | CDR3 | | | | | |
|---|---|---|---|---|---|---|---|---|---|---|---|---|---|
| Kabat # | | 55 | 56 | 57 | 58 | 59 | 60 | 93 | 94 | 95 | 101 | 102 | 103 |
| F0103265A11 | | G | Y | T | N | Y | V | S | L | S | R | Y | L |
| Rank | 1 | M | W | R | T | | N | A | A | | V | | Q |
| hits | 2 | N | | V | S | | T | | | | L | | |
| stage I | 3 | | | W | A | | | | | | T | | |
| | 4 | | | | L | | | | | | | | |
| | 5 | | | | | | | | | | | | |

| | | | | | | | | | | | | |
|---|---|---|---|---|---|---|---|---|---|---|---|---|
| Stage II design | G | YW | TV | NT | Y | VN | SA | L | S | RV | Y | LQ |

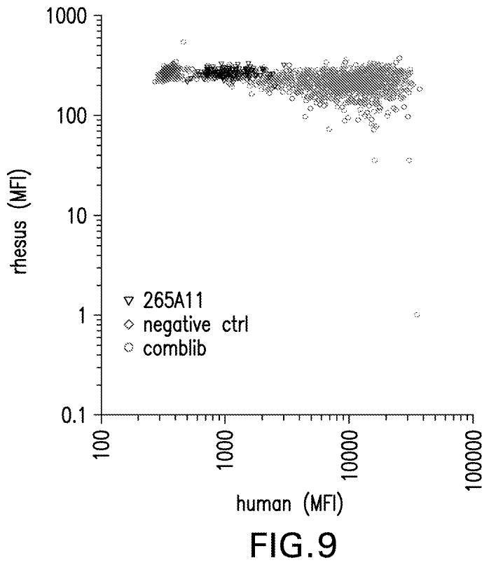

Crude periplasmic of 2016 clones of the stage II combinatorial library were prepared and screened in binding FACS on huNav1.7α and rhNav1.7α. A large fraction of the variants displayed improved binding to huNav1.7α compared to the parental F0103265A11 (FIG. 9), indicating that the library design successfully captured and improved the promise of the stage I library. No improvements for binding to rhNav1.7α were observed for stage II, in line with the observations during stage I. The sequence analysis of 288 hits is summarized in Table 13. Compared to a randomly picked reference sample, the top 25% of the hits are enriched for the A31R, V60N and S93A substitutions but display a lower proportion of the N30Y, 151W, S53W, T57V and N58T substitutions. Compared to a randomly picked reference sample, the bottom 25% of the hits are enriched for the T57V, S93A and L103Q substitutions but display a lower proportion of the N30Y, I51W and S53W substitutions.

TABLE 13
Summary sequence analysis of F0103265A11 screening stage II affinity maturation libraries

| | CDR1 | | CDR2 | | | | | | CDR3 | | |
|---|---|---|---|---|---|---|---|---|---|---|---|
| Kabat # | N30Y | A31R | I51W | S53W | Y56W | T57V | N58T | V60N | S93A | R101V | L103Q |
| Reference | 22% | 30% | 27% | 37% | 30% | 45% | 55% | 37% | 38% | 18% | 55% |
| sample | | | | | | | | | | | |
| Top 25% of | 11% | 51% | 11% | 17% | 27% | 37% | 41% | 79% | 56% | 14% | 60% |
| hits | | | | | | | | | | | |
| Bottom 25% of | 11% | 30% | 11% | 25% | 25% | 56% | 51% | 41% | 62% | 27% | 68% |
| hits | | | | | | | | | | | |

A number of combinatorial affinity maturation variants of F0103265A11 were then characterized in detail in binding FACS and electrophysiology (Table 14). Most variants displayed clear improvements in binding EC50 and Bmax values on huNav1.7α, compared to parental F0103265A11. This became even more pronounced when huNav1.7α was expressed in the absence of Navβ-subunits: no binding was observed for parental 265A11, whereas many affinity maturation variants showed clear binding curves to the HEKa-only line. The previously observed β-subunit dependency of F0103265A11 was improved by the affinity maturation process. Clear improvements in functional inhibition of the ion channel were observed (last column of Table 14), compared to the marginal functional inhibition observed in the past for parental F0103265A11.

TABLE 14
Summary binding and functional characterization of F0103265A11 affinity variants
Part 1

| | Kabat # (mutations vs. F01033265A11) | | | | | | | | | | |
|---|---|---|---|---|---|---|---|---|---|---|---|
| ID # | N30 | A31 | I51 | S53 | Y56 | T57 | N58 | V60 | S93 | R101 | L103 |
| F010301458 | . | R | . | . | W | . | . | N | A | . | Q |
| F010301463 | . | R | . | . | . | . | . | N | A | . | Q |
| F010301080 | . | . | . | W | . | V | T | N | . | . | . |
| F010301129 | . | . | . | . | W | . | . | N | A | . | Q |
| F010301162 | . | R | . | . | W | . | . | N | . | . | Q |
| F010301191 | . | R | . | . | W | . | . | N | A | V | Q |
| F010301055 | . | . | . | W | . | V | . | . | . | . | . |
| F010301139 | . | R | W | . | . | V | . | N | . | . | Q |
| F010301090 | . | . | . | . | W | . | T | N | . | . | Q |
| F010301188 | . | R | . | . | . | . | . | N | . | . | Q |
| F010301175 | . | R | . | . | . | . | T | . | . | . | . |
| F010301111 | . | . | . | . | . | V | . | N | A | . | . |
| F010301059 | . | . | . | . | . | V | . | N | A | . | Q |
| F010300535 | . | . | . | W | . | . | . | . | . | . | . |
| F010301126 | . | R | . | . | . | V | T | N | . | . | . |
| F010301113 | . | R | . | . | . | . | . | N | . | V | Q |
| F010301099 | . | . | . | . | W | V | T | . | . | . | . |
| F010300536 | . | . | . | . | . | . | T | . | . | . | . |
| F010301232 | . | R | . | . | . | . | T | . | A | . | Q |
| F010301138 | Y | . | . | . | . | . | . | N | A | . | Q |
| F010300534 | . | . | . | . | . | V | . | . | . | . | . |
| F0103265A11 | . | . | . | . | . | . | . | . | . | . | . |

Part 2

| | | HEKa/β1 | | | |
|---|---|---|---|---|---|
| | | (SEQ ID | | | |
| | HEK FlpIn | NO: 40) | | HEKa | |
| | Nav1.7α + β1- | huNav1.7α | | Nav1.7α | HEKa/β1 |
| | β2-β3 | (SEQ ID | Bmax | (SEQ ID | (SEQ ID |
| | (SEQ ID NO: | NO: 1) | relative | NO: 1) | NO: 40) |
| ID # | 3) EC50 [M] | EC50 [M] | to ′535 | EC50 [M] | IC50 [M] |
| F010301458 | 4.1E−09 | 2.2E−09 | 120% | 8.6E−09 | ND |
| F010301463 | 4.3E−09 | 2.5E−09 | 120% | 2.9E−08 | ND |
| F010301080 | 5.1E−09 | 3.0E−09 | 122% | 2.6E−08 | 1.4E−08 |
| F010301129 | ND | 3.6E−09 | 103% | ND | 1.3E−07 |
| F010301162 | ND | 3.8E−09 | 115% | ND | 3.2E−08 |
| F010301191 | ND | 4.1E−09 | 119% | ND | 6.3E−08 |
| F010301055 | ND | 4.4E−09 | 117% | ND | ND |
| F010301139 | ND | 4.7E−09 | 117% | ND | ND |
| F010301090 | ND | 4.8E−09 | 102% | ND | 2.2E−08 |
| F010301188 | ND | 5.1E−09 | 104% | ND | ND |
| F010301175 | ND | 5.4E−09 | 111% | ND | ND |
| F010301111 | ND | 5.4E−09 | 99% | ND | ND |
| F010301059 | ND | 5.4E−09 | 99% | ND | ND |
| F010300535 | 1.6E−08 | 5.5E−09 | 100% | 7.2E−07 | ND |
| F010301126 | ND | 5.6E−09 | 118% | ND | ND |
| F010301113 | ND | 5.7E−09 | 111% | ND | ND |
| F010301099 | ND | 7.4E−09 | 87% | ND | ND |
| F010300536 | 2.7E−08 | 9.0E−09 | 77% | 6.6E−07 | ND |
| F010301232 | ND | 9.3E−09 | 112% | ND | ND |
| F010301138 | ND | 9.7E−09 | 107% | ND | ND |
| F010300534 | 7.2E−08 | 1.1E−08 | 62% | — | ND |
| F0103265A11 | 7.7E−08 | 1.1E−08 | 34% | — | ND |

—, no binding detected;
ND, not determined

### Affinity Maturation of F0103265B04

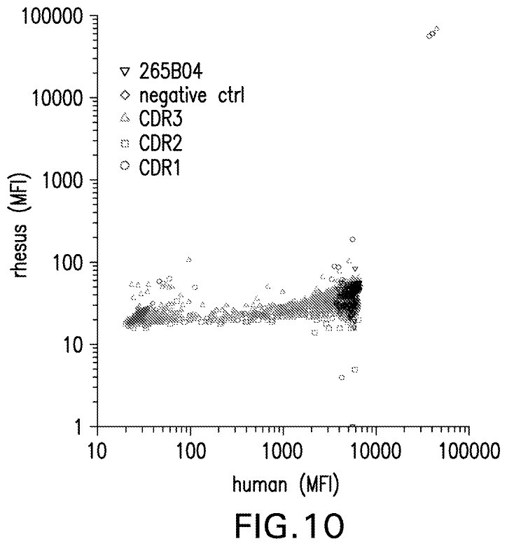

A pooled single site saturation library of F0103265B04 was constructed and crude periplasmic extracts of 2016 individual clones were prepared and screened in binding FACS on huNav1.7a and rhNav1.7α. No clones with a single mutation in CDR3, CDR2 or CDR1 residues showed an improved binding to huNav1.7a or rhNav1.7α (FIG. 10). The outliers in the top right quadrant of FIG. 10 was determined to be a contamination with F0103240B04, a 132 binding ISVD.

### Affinity Maturation of F0103387G05

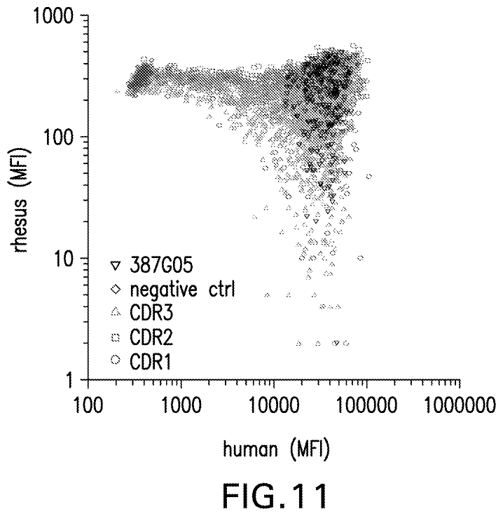

A pooled single site saturation library of F0103387G05 was constructed and crude periplasmic extracts of 3360 individual clones were prepared and screened in binding FACS on huNav1.7α and rhNav1.7α. Clones with a single mutation of CDR2, CDR3 or CDR1 residues showed weakly improved binding to huNav1.7α, but not to rhNav1.7α (FIG. 11).

Sequence analysis of 384 hits revealed an enrichment for certain positions and mutations, but as there were no outspoken improvements in binding observed, it was decided to first characterize a number of stage I variants rather than combining these in a stage II combinatorial library. Binding FACS experiments (Table 15) revealed that most of the tested variants were comparable to parental F0103387G05. Interestingly, a number of CDR1- and CDR2-based (Kabat positions 23, 53, 54 and 58) mutations, all substitutions of Asp with Gly, displayed subtle improvements compared to parental F0103387G05. Combinations of these substitutions further improved the binding in a subtle way (Table 15). Combinations of these substitutions further improved the binding in a subtle way with D23A and D58G substitutions contributing the most (Table 15), resulting in the selection of F0103301563 as the preferred variant.

TABLE 15
Summary binding characterization of F0103387G05 affinity variants

| | | CHO FlpIn | HEKa/β1 | |
|---|---|---|---|---|
| | | huNav1.7α + | (SEQ ID NO: 40) | HEKa |
| | | β1-β2-β3 | huNav1.7α | huNav1.7α |
| | | (SEQ ID NO: 3) | (SEQ ID NO: 1) | (SEQ ID NO: 1) |
| ID # | Mutations vs. 387G05 | EC50 [M] | EC50 [M] | EC50 [M] |
| F010301559 | D23A, D54G, D58G | 1.8E−09 | 9.2E−10 | 8.7E−10 |
| F010301558 | D23A, D53G, D58G | 1.8E−09 | 9.3E−10 | 1.5E−09 |
| F010301563 | D23A, D58G | 1.9E−09 | 1.0E−09 | 1.5E−09 |
| F010301560 | D53G, D54G, D58G | 2.1E−09 | 1.1E−09 | 1.2E−09 |
| F010301565 | D53G, D58G | 2.1E−09 | 1.0E−09 | 1.4E−09 |
| F010301566 | D54G, D58G | 2.2E−09 | 1.0E−09 | 1.2E−09 |
| F010301556 | D23A, D53G, D54G, D58G | 2.3E−09 | 1.4E−09 | 1.8E−09 |
| F010301561 | D23A, D53G | 2.5E−09 | 1.2E−09 | 2.2E−09 |
| F010301562 | D23A, D54G | 2.5E−09 | 1.9E−09 | 1.2E−09 |
| F010301346 | D58G | 3.4E−09 | 1.6E−09 | 1.5E−09 |
| F010301564 | D53G, D54G | 3.5E−09 | 2.4E−09 | 1.9E−09 |
| F010301350 | D58V | 3.5E−09 | 1.7E−09 | 1.6E−09 |
| F010301314 | D23A | 3.5E−09 | 1.8E−09 | 1.9E−09 |
| F010301557 | D23A, D53G, D54G | 3.8E−09 | 2.1E−09 | 2.0E−09 |
| F010301367 | D53G | 4.2E−09 | 1.9E−09 | 2.2E−09 |
| F010301344 | D54G | 4.2E−09 | 2.1E−09 | 1.3E−09 |
| F010301372 | S100dG | 4.5E−09 | 2.3E−09 | 2.5E−09 |
| F010301445 | S100dY | 4.6E−09 | 2.0E−09 | 2.3E−09 |
| F0103387G05 | | 4.7E−09 | 1.7E−09 | 2.7E−09 |
| F010301418 | Y102W | 4.9E−09 | ND | ND |
| F010301328 | T57V | 5.0E−09 | ND | ND |
| F010301409 | H100fV | 5.1E−09 | ND | ND |
| F010301387 | V100A | 5.2E−09 | ND | ND |
| F010301360 | A56T | 5.2E−09 | ND | ND |
| F010301309 | L29V | 5.4E−09 | ND | ND |
| F010301313 | R35K | 5.5E−09 | ND | ND |
| F010301304 | I31W | 5.7E−09 | ND | ND |
| F010301440 | G100hT | 5.7E−09 | ND | ND |
| F010301301 | I31T | 7.3E−09 | ND | ND |
| F010301335 | R50V | 7.9E−09 | ND | ND |
| F010301425 | G100bV | 1.5E−08 | ND | ND |
| F010301416 | T101Q | — | ND | ND |

—, no binding detected;
ND, not determined

### Affinity Maturation of F0103362B08

A pooled single site saturation library of F0103362B08 was constructed and crude periplasmic extracts of 4032 individual clones were prepared and screened in binding FACS on huNav1.7α and rhNav1.7α. Clones with a single mutation of CDR2, CDR3 or CDR1 residues showed weakly improved binding to huNav1.7α, but not to rhNav1.7α (FIG. 12).

Sequence analysis of 326 hits revealed an enrichment for certain positions and mutations, but as there were no outspoken improvements in binding observed, it was decided to first characterize a number of stage I variants rather than combining these in a stage II combinatorial library. Binding FACS experiments (Table 16) revealed that most of the tested variants were comparable to parental F0103362B8. A number of mutations (Kabat positions 50, 97, 99 and 1000, consistently displayed subtle improvements compared to parental 362B08 across two different huNav1.7α cell lines.

TABLE 16
Summary binding characterization of F0103362B08 affinity variants

| | | CHO FlpIn | HEKa/β1 | |
|---|---|---|---|---|
| | | huNav1.7α + | (SEQ ID NO: 40) | HEKa |
| | | β1-β2-β3 | huNav1.7α | huNav1.7α |
| | | (SEQ ID NO: 3) | (SEQ ID NO: 1) | (SEQ ID NO: 1) |
| ID # | Mutations vs. 362B08 | EC50 [M] | EC50 [M] | EC50 [M] |
| F010301595 | D97A | 6.2E−08 | 1.7E−08 | ND |
| F010301606 | Y100fK | 6.5E−08 | 2.4E−08 | ND |
| F010301627 | G50A | 7.6E−08 | 2.3E−08 | ND |
| F010301598 | G99Q | 8.9E−08 | 2.9E−08 | ND |
| F010301607 | Y100fA | 9.0E−08 | 3.0E−08 | ND |
| F010301591 | G99K | 9.3E−08 | 4.3E−08 | ND |
| F010301574 | G35A | 1.0E−07 | 2.9E−08 | ND |
| F010301594 | R100aK | 1.1E−07 | 2.9E−08 | ND |
| F010301584 | W52aT | 1.1E−07 | 4.2E−08 | ND |
| F010301593 | R96K | 1.1E−07 | 4.0E−08 | ND |
| F010301585 | V56P | 1.1E−07 | 5.3E−08 | ND |
| F010301567 | F29Y | 1.1E−07 | 2.4E−08 | ND |
| F0103362B08 | | 1.1E−07 | 3.4E−08 | 1.9E−08 |
| F010301578 | S30G | 1.2E−07 | 3.0E−08 | ND |
| F010301629 | G50S | 1.2E−07 | 4.2E−08 | ND |
| F010301589 | I55P | 1.2E−07 | 4.1E−08 | ND |
| F010301579 | R27K | 1.3E−07 | 3.6E−08 | ND |
| F010301596 | G99A | 1.4E−07 | 5.0E−08 | ND |
| F010301621 | Y100fG | 1.5E−07 | 3.4E−08 | ND |
| F010301617 | F100cQ | 1.7E−07 | 4.4E−08 | ND |
| F010301586 | W52aA | 1.7E−07 | 5.1E−08 | ND |
| F010301612 | Y100fT | 1.7E−07 | 4.4E−08 | ND |
| F010301622 | Y100fD | 1.7E−07 | 4.0E−08 | ND |
| F010301580 | R27T | 1.8E−07 | 3.5E−08 | ND |
| F010301619 | F100cK | 1.8E−07 | 4.7E−08 | ND |
| F010301618 | Y102H | 1.9E−07 | 3.9E−08 | ND |
| F010301609 | Y100fQ | 1.9E−07 | 3.7E−08 | ND |
| F010301604 | R100aQ | 1.9E−07 | 5.2E−08 | ND |
| F010301592 | R100aS | 2.0E−07 | 6.7E−08 | ND |
| F010301568 | G35T | 3.1E−07 | 1.1E−07 | ND |
| F010301657 | A14P, D97A, G99Q, Y100fK | ND | 1.6E−08 | 2.0E−08 |
| F010301658 | A14P, G50A, D97A, G99Q, Y100fK | ND | 2.0E−08 | 2.3E−08 |
| F010301659 | A14P, G50A, D97A | ND | 1.2E−08 | 1.1E−08 |
| F010301661 | A14P, G50A, Y100fK | ND | 1.8E−08 | 1.6E−08 |
| F010301662 | A14P, D97A, G99Q | ND | 1.7E−08 | 1.9E−08 |
| F010301663 | A14P, D97A, Y100fK | ND | 1.4E−08 | 1.3E−08 |
| F010301664 | A14P, G99Q, Y100fK | ND | 1.8E−08 | 1.3E−08 |
| F010301665 | A14P, G50A, D97A, Y100fK | ND | 1.1E−08 | 8.8E−09 |
| F010301666 | A14P, G50A, G99Q, Y100fK | ND | 1.4E−08 | 1.0E−08 |

ND, not determined

### Affinity Maturation of F0103464B09

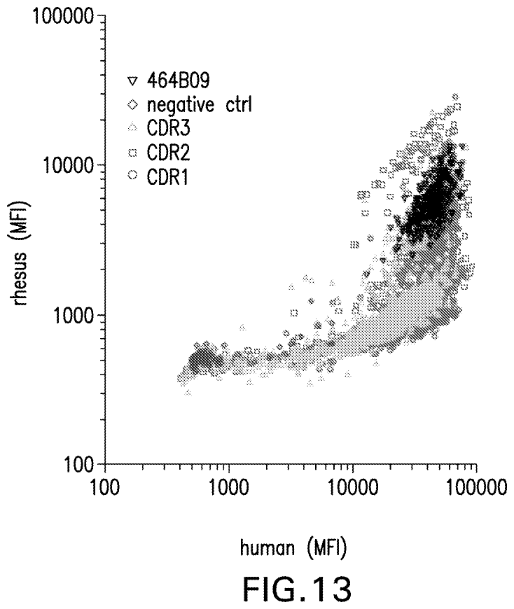

A pooled single site saturation library of F0103464B09 was constructed and crude periplasmic extracts of 3356 individual clones were prepared and screened in binding FACS on huNav1.7α and rhNav1.7α. Clones with a single mutation of mainly CDR2 residues showed weakly improved binding to rhNav1.7α, but hardly not to huNav1.7α (FIG. 13).

Sequence analysis of 186 hits revealed an enrichment for certain positions and mutations, particularly in CDR1 and CDR2. It was decided to first characterize a number of stage I variants based on their sequence enrichment in the hits and/or improved binding vs. parental controls (Table 17). Compared to parental F0103464B09, a number of the tested substitutions clearly improved binding to rhNav1.7α in terms of Bmax while being neutral for binding to huNav1.7α.

TABLE 17
Summary binding characterization of F0103464B09 affinity variants

| | | CHO FlpIn | CHO FlpIn | CHO FlpIn |
|---|---|---|---|---|
| | | rhNav1.7α + | rhNav1.7α + | huNav1.7α + |
| | | β1-β2-β3 | β1-β2-β3 | β1-β2-β3 |
| | Mutations vs. | (SEQ ID NO: 4) | (SEQ ID NO: 4) | (SEQ ID NO: 3) |
| ID # | F103464B09 | EC50 [M] | Bmax | EC50 [M] |
| F010301892 | V33L | 9.1E−09 | 100% | 4.E−09 |
| F010301885 | N53E | 1.8E−08 | 100% | 8.0E−09 |
| F010301881 | G54W | 5.8E−09 | 99% | 4.2E−09 |
| F010301888 | S26H | 8.7E−09 | 95% | 5.3E−09 |
| F010301889 | S95A | 8.5E−09 | 92% | 5.4E−09 |
| F010301878 | G54E | 2.7E−08 | 83% | 7.1E−09 |
| F010301886 | N58Q | 1.5E−08 | 81% | 4.3E−09 |
| F010301867 | A28Q | 2.2E−08 | 74% | 4.8E−09 |
| F010301880 | G54S | 2.4E−08 | 73% | 4.9E−09 |
| F010301890 | T35V | 2.0E−08 | 58% | 5.0E−09 |
| F0103464B09 | | 3.3E−08 | 54% | 5.4E−09 |
| F010301887 | R31Q | 5.9E−08 | 51% | 7.5E−09 |
| F010301883 | I51V | 3.4E−08 | 48% | 4.8E−09 |
| F010301884 | N53A | 5.0E−08 | 47% | 6.0E−09 |
| F010301891 | T57V | 2.6E−08 | 45% | 4.7E−09 |
| F010301882 | I30V | 4.1E−08 | 43% | 4.5E−09 |
| F010301879 | G54Q | ND | ND | ND |

ND, not determined

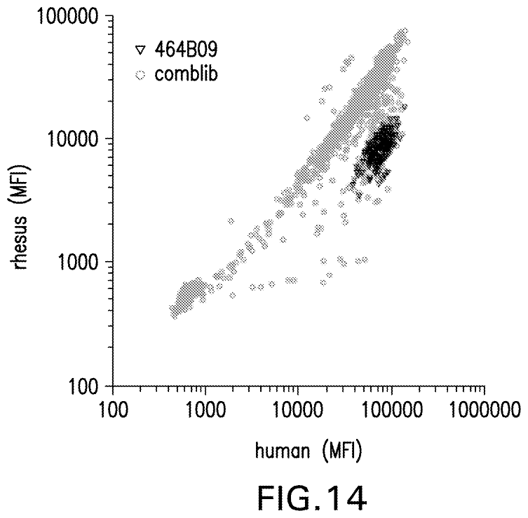

Based on these observations, a combinatorial library was generated with a diversity of 320 different variants, as summarized by Table 17. Crude periplasmic of 2880 clones of the stage II combinatorial library were prepared and screened in binding FACS on huNav1.7α and rhNav1.7α. A large fraction of the variants display improved binding to rhNav1.7α compared to the parental F0103464B09 (FIG. 14), indicating that the library design successfully captured and improved the promise of the stage I library. No outspoken improvements for binding to huNav1.7α were observed for stage II, in line with the observations during stage I.

The sequence analysis of 273 hits (per 96-well plate, each time top three hits on huNav1.7α and top seven hits on rhNav1.7α) is summarized in Table 18. Compared to a randomly picked reference sample, the V33L, G54W and S95A substitutions are underrepresented in the top three hits on huNav1.7α and rhNav1.7α. As such, the variants with these substitutions were excluded from further analysis. Furthermore, 38/96 (40%) of the top 3 hits on huNav1.7α matched the parental F0103464B09 sequence, again suggesting that no major improvements on huNav1.7α could be expected from this library. As no outspoken sequence enrichments could be observed from Table 18, the following criteria were applied to further narrow down the number of variants for detailed characterization:
sequenced multiple times and at least once present in both top 2 hits on huNav1.7α and rhNav1.7α;
no deamidation motif on position 53;
less than 5 mutations compared to parental F0103464B09.
   The resulting combinatorial variants (Table 19) were supplemented with one variant carrying V33L substitution, as this is one of the most promising single substitutions (Table 17). These variants were combined with a two variable sequence optimization substitutions R39Q and A63V in a background containing a large number of sequence optimization substitutions (L11V, T24A, T25S, V40A, E44Q, F62S, S68T, M77T, T79Y, R81Q, S82aN, N82bS, K83R, G88A, V89L, N99S).

TABLE 18
Summary sequence analysis of F103464B09 screening stage II affinity maturation libraries

| | CDR1 | | | CDR2 | | | | | | CDR3 |
|---|---|---|---|---|---|---|---|---|---|---|
| Kabat # | S26H | A28Q | V33L | N53E | G54W | G54E | G54S | G54Q | N58Q | S95A |
| Reference | 55% | 41% | 48% | 48% | 10% | 24% | 28% | 21% | 72% | 52% |
| sample | | | | | | | | | | |
| Top 3 hits | 47% | 58% | 4% | 42% | 0% | 20% | 29% | 11% | 62% | 9% |
| on huNav1.7 | | | | | | | | | | |
| Top 3 hits | 51% | 58% | 19% | 31% | 1% | 21% | 19% | 15% | 85% | 36% |
| on rhNav1.7 | | | | | | | | | | |

TABLE 19
Summary of selected F103464B09 affinity variants

| | Kabat # (mutations vs. F103464B09) | | | | | |
|---|---|---|---|---|---|---|
| ID | S26 | A28 | V33 | N53 | G54 | N58 |
| A28Q G54E | . | Q | . | . | E | . |
| A28Q G54E N58Q | . | Q | . | . | E | Q |
| A28Q N53E G54S N58Q | . | Q | . | E | S | Q |
| S26H A28Q G54E N58Q | H | Q | . | . | E | Q |
| S26H A28Q N53E N58Q | H | Q | . | E | . | Q |
| S26H N53E G54S N58Q | H | . | . | E | S | Q |
| S26H N53E N58Q | H | . | . | E | . | Q |
| S26H V33L N53E G54S | H | . | L | E | S | . |

In the course of the F0103464B09 sequence optimization process subtle drops in binding to rhNav1.7α were observed for the following substitutions: R39Q, A63V, T79Y, R81Q, and N99S. R39Q substitution also resulted in a subtle drop in binding to huNav1.7α. The combination of these, as present in the background in which the combinatorial variation was introduced, resulted in the complete abolishment of binding to rhNav1.7α for the controls that do not carry any of the affinity maturation substitutions (F010302365, F010302366 and F010302368 in Table 20) and the same was observed for the variants combining the A28Q G54E substitutions. Less outspoken, none of the variants combining the A28Q G54E N58Q, S26H A28Q G54E N58Q, or A28Q N53E G54S N58Q substitutions reached maximum binding levels to rhNav1.7α (Table 20). A similar observation was made for the variants combining the S26H V33L N53E G54S substitutions, which also resulted in a drop in binding EC50 to huNav1.7α. The three remaining combinations S26H N53E N58Q, S26H N53E G54S N58Q and S26H A28Q N53E N58Q were highly comparable for their binding to huNav1.7α and rhNav1.7α (Table 20). The S26H N53E N58Q combination was then selected as it achieves the same binding improvements with one mutation less than the two others.

TABLE 20
Summary binding characterization of F0103464B09 combinatorial
affinity and sequence optimization variants
Part 1

| | Substitutions vs. F103464B09 (L11V, T24A, T25S, V40A, E44Q, F62S, | | | | | | | |
|---|---|---|---|---|---|---|---|---|
| | S68T, M77T, T79Y, R81Q, S82aN, N82bS, K83R, G88A, V89L, N99S) | | | | | | | |
| ID # | S26 | A28 | V33 | R39 | N53 | G54 | N58 | A63 |
| F010302365 | . | . | . | . | . | . | . | . |
| F010302368 | . | . | . | Q | . | . | . | V |
| F010302366 | . | . | . | Q | . | . | . | . |
| F010302341 | . | Q | . | Q | . | E | . | . |
| F010302357 | . | Q | . | Q | . | E | . | V |
| F010302333 | . | Q | . | . | . | E | . | . |
| F010302349 | . | Q | . | . | . | E | . | V |
| F010302364 | H | . | L | Q | E | S | . | V |
| F010302348 | H | . | L | Q | E | S | . | . |
| F010302356 | H | . | L | . | E | S | . | V |
| F010302340 | H | . | L | . | E | S | . | . |
| F010302339 | H | . | . | . | E | . | Q | . |
| F010302347 | H | . | . | Q | E | . | Q | . |
| F010302355 | H | . | . | . | E | . | Q | V |
| F010302363 | H | . | . | Q | E | . | Q | V |
| F010302337 | H | Q | . | . | E | . | Q | . |
| F010302345 | H | Q | . | Q | E | . | Q | . |
| F010302353 | H | Q | . | . | E | . | Q | V |
| F010302361 | H | Q | . | Q | E | . | Q | V |
| F010302334 | . | Q | . | . | . | E | Q | . |
| F010302342 | . | Q | . | Q | . | E | Q | . |
| F010302350 | . | Q | . | . | . | E | Q | V |
| F010302358 | . | Q | . | Q | . | E | Q | V |
| F010302336 | H | Q | . | . | . | E | Q | . |
| F010302344 | H | Q | . | Q | . | E | Q | . |
| F010302352 | H | Q | . | . | . | E | Q | V |
| F010302360 | H | Q | . | Q | . | E | Q | V |
| F010302338 | H | . | . | . | E | S | Q | . |
| F010302346 | H | . | . | Q | E | S | Q | . |
| F010302354 | H | . | . | . | E | S | Q | V |
| F010302362 | H | . | . | Q | E | S | Q | V |
| F010302335 | . | Q | . | . | E | S | Q | . |
| F010302343 | . | Q | . | Q | E | S | Q | . |
| F010302351 | . | Q | . | . | E | S | Q | V |
| F010302359 | . | Q | . | Q | E | S | Q | V |

Part 2

| | | | CHO FlpIn | CHO FlpIn |
|---|---|---|---|---|
| | CHO FlpIn | | rhNav1.7α + | rhNav1.7α + |
| | huNav1.7α + | CHO FlpIn | β1- β2-β3 | β1- β2-β3 |
| | β1-β2-β3 (SEQ | huNav1.7α + β1- | (SEQ ID | (SEQ ID |
| | ID NO: 3) | β2-β3 (SEQ ID | NO: 4) | NO: 4) |
| ID # | EC50 [M] | NO: 3) Bmax | EC50 [M] | Bmax |
| F010302365 | 3.1E−09 | 100% | — | — |
| F010302368 | 3.9E−09 | 100% | — | — |
| F010302366 | 3.7E−09 | 100% | — | — |
| F010302341 | 5.3E−09 | 100% | — | — |
| F010302357 | 5.3E−09 | 100% | — | — |
| F010302333 | 4.1E−09 | 100% | — | — |
| F010302349 | 4.4E−09 | 100% | — | — |
| F010302364 | 7.2E−09 | 100% | 1.7E−08 | 74% |
| F010302348 | 6.7E−09 | 100% | 1.4E−08 | 95% |
| F010302356 | 5.3E−09 | 100% | 1.2E−08 | 81% |
| F010302340 | 4.6E−09 | 100% | 1.3E−08 | 96% |
| F010302339 | 4.3E−09 | 100% | 6.8E−09 | 100% |
| F010302347 | 4.6E−09 | 100% | 9.6E−09 | 100% |
| F010302355 | 3.5E−09 | 100% | 6.8E−09 | 97% |
| F010302363 | 4.2E−09 | 100% | 9.3E−09 | 96% |
| F010302337 | 3.9E−09 | 100% | 6.8E−09 | 100% |
| F010302345 | 4.9E−09 | 100% | 1.2E−08 | 96% |
| F010302353 | 4.2E−09 | 100% | 7.9E−09 | 93% |
| F010302361 | 4.8E−09 | 100% | 1.1E−08 | 92% |
| F010302334 | 4.0E−09 | 100% | 1.1E−08 | 70% |
| F010302342 | 4.0E−09 | 100% | 1.6E−08 | 61% |
| F010302350 | 3.5E−09 | 100% | 1.5E−08 | 46% |
| F010302358 | 4.5E−09 | 100% | 3.7E−08 | 37% |
| F010302336 | 3.6E−09 | 100% | 9.7E−09 | 79% |
| F010302344 | 4.3E−09 | 100% | 1.3E−08 | 78% |
| F010302352 | 3.6E−09 | 100% | 1.1E−08 | 60% |
| F010302360 | 3.5E−09 | 100% | 1.5E−08 | 58% |
| F010302338 | 3.4E−09 | 100% | 7.9E−09 | 100% |
| F010302346 | 5.0E−09 | 100% | 1.2E−08 | 99% |
| F010302354 | 4.2E−09 | 100% | 7.9E−09 | 96% |
| F010302362 | 5.4E−09 | 100% | 1.4E−08 | 89% |
| F010302335 | 4.1E−09 | 100% | 1.3E−08 | 84% |
| F010302343 | 6.4E−09 | 100% | 1.8E−08 | 84% |
| F010302351 | 5.6E−09 | 100% | 1.5E−08 | 72% |
| F010302359 | 6.4E−09 | 100% | 2.4E−08 | 66% |

### Example 4

Competitive Binding to huNav1.7α

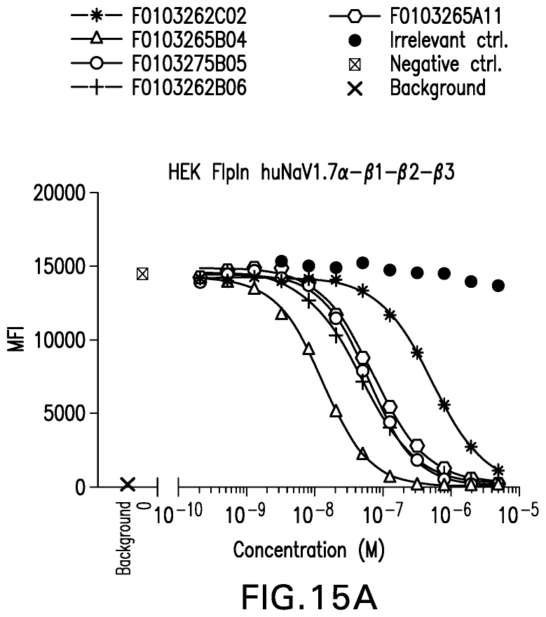

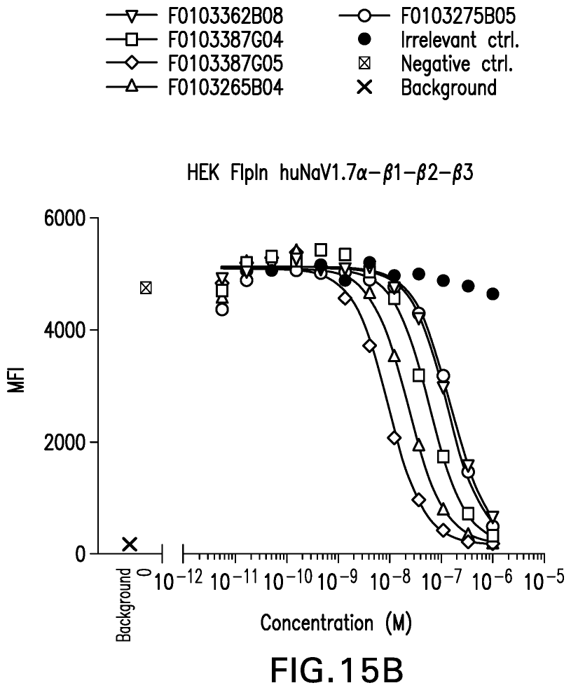

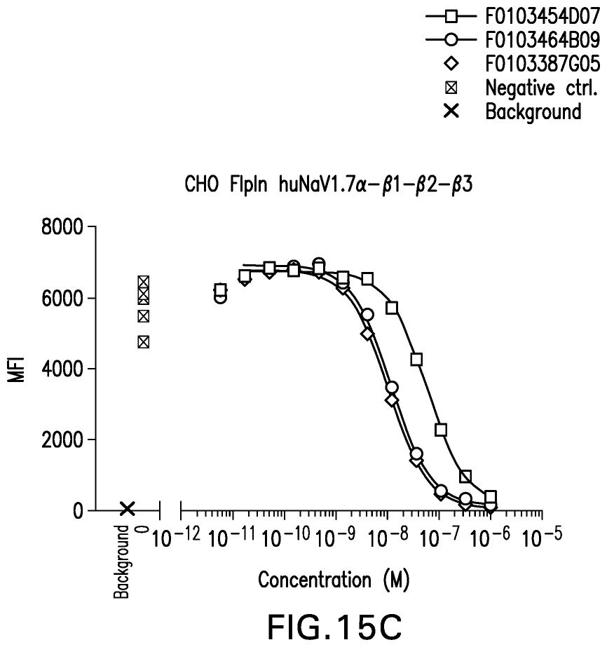

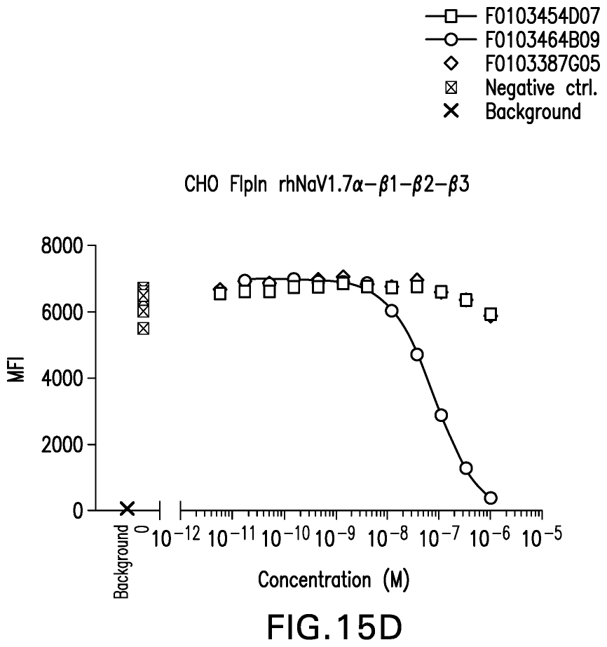

Competition FACS assays were performed with CMYC3-tagged ISVD F0103265B04 or F0103275B05(N93R) affinity maturation variant on a HEK FlpIn huNav1.7α+β1−β2−β3 transgenic cell line. Briefly, cells were resuspended in FACS buffer (PBS, 2% FBS, 0.05% NaN₃) and 1×10⁵ cells/well were transferred to 96-well V-bottom plates. Cells were subsequently resuspended in a 100 μL mixture of purified ISVD (dilution series) and CMYC3-tagged ISVD F0103265B04 (at a concentration equivalent to EC30) followed by incubation for 1.5 hours at 4° C. Residual binding of CMYC3-tagged ISVD F0103265B04 was detected with 1004 murine anti-CMYC (1/250 dilution) (Bio-Rad, catalog #MCA2200) followed by PE-conjugated goat anti-murine (Jackson Immunoresearch, catalog #115-116-071). Between each step, the cells were centrifuged for 5 minutes at 200 g and washed with 100 μL/well FACS buffer. Prior to the read-out, the samples were resuspended in 5 nM TOPRO3 (Molecular probes, catalog #T3605) to exclude dead cells. F0103262CO2, F0103262B06, F0103265A11, F0103265B04, F0103275B05, F0103362B08, and F0103387G04 all compete with F0103265B04 for binding to huNav1.7α, in contrast to an irrelevant control ISVD (IRR) (see Table 21 and FIG. 15A, FIG. 15B, FIG. 15C, and FIG. 15D). The data shown in the figures and summarized in Table 20 suggests that all extracellular anti-Nav1.7α leads bind to an overlapping footprint.

TABLE 21

| | HEK FlpIn | CHO FlpIn | CHO FlpIn |
|---|---|---|---|
| | huNav1.7α + | huNav1.7α + | rhNav1.7α + |
| | β1-β2-β3 | β1-β2-β3 | β1-β2-β3 |
| | (SEQ ID NO: 3) | (SEQ ID NO: 3) | (SEQ ID NO: 4) |
| | vs. EC30 of | vs. EC25 of | vs. EC40 of |
| | F103265B04 | F103275B05(N93R) | F103275B05(N93R) |
| ID # | IC50 [M] | IC50 [M] | IC50 [M] |
| F0103262C02 | 5.3E−07 | ND | ND |
| F0103262B06 | 4.9E−08 | ND | ND |
| F0103275B05 | 9.8E−08 | ND | ND |
| F0103265B04 | 1.8E−08 | ND | ND |
| F0103265A11 | 7.5E−08 | ND | ND |
| F0103362B08 | 1.5E−07 | ND | ND |
| F0103387G05 | 9.4E−09 | 1.0E−08 | — |
| F0103387G04 | 5.9E−08 | ND | ND |
| F0103464B09 | ND | 1.2E−08 | 7.9E−08 |
| F0103454D07 | ND | 5.8E−08 | — |

ND, not determined

### Example 5

Binding to huNav1.7α-Nav1.5 Chimeras

FACS binding studies (as described above) were performed on HEK293T cells transiently transfected with expression vectors encoding a huNav1.7α or rhNav1.7α fused at the C-terminus via a P2A viral peptide linker to a single polypeptide encoding sodium channel beta subunits Navβ1, Navβ2, and Navβ3 in tandem (β1-β2-β3; SEQ ID NO:21). Similarly, HEK293T cells transiently transfected with expression vectors encoding chimeric variants of huNav1.7α in which individual domains are replaced by their huNav1.5α counterparts (chimeras 1 to 4 in FIG. 16) or with chimeric variants of huNav1.5 in which individual domains are replaced by their huNav1.7α counterparts (chimeras 5 to 8 in FIG. 16) fused at the C-terminus via a P2A viral peptide linker to β1-β2-β3 as above. See Table 7 for description of the expression vectors encoding the chimeras, Table 21 and FIG. 16).

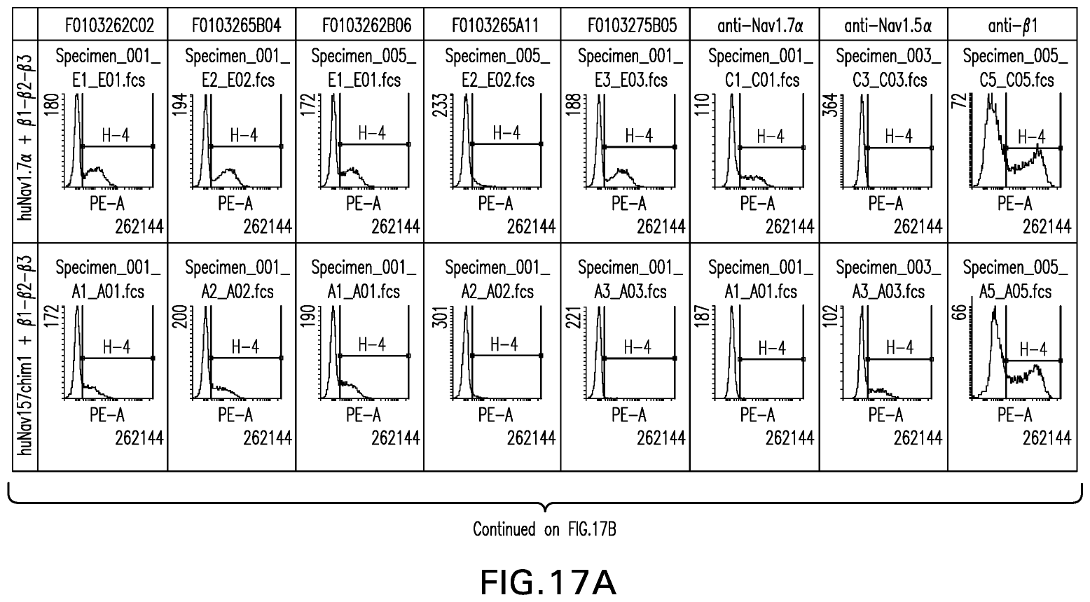

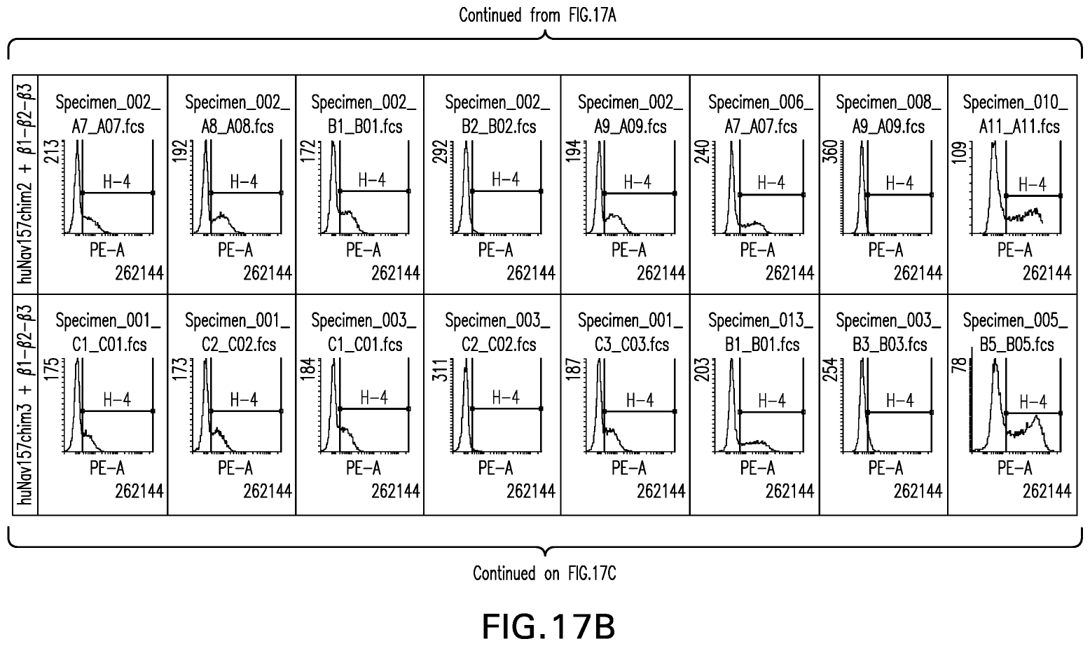

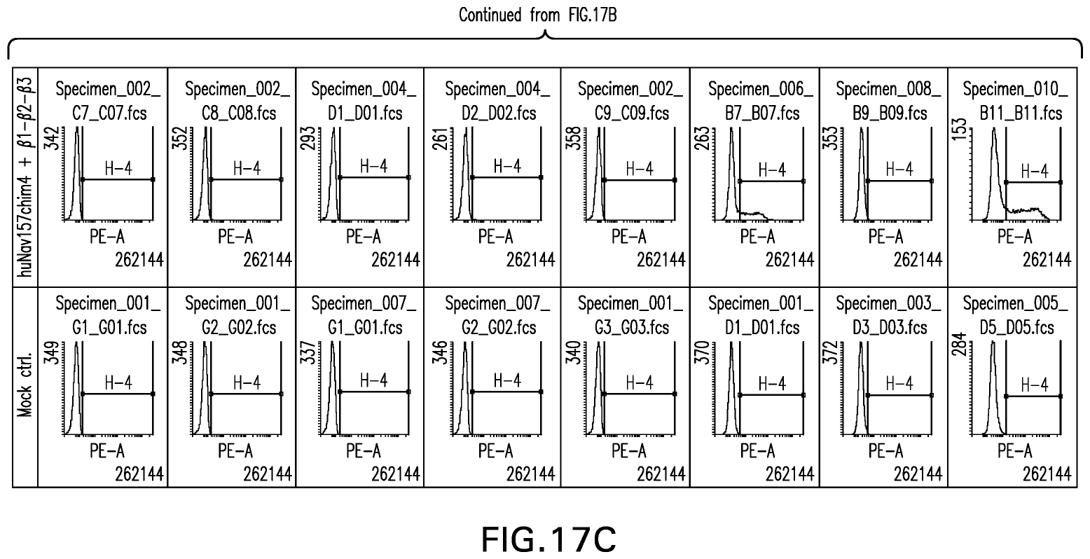

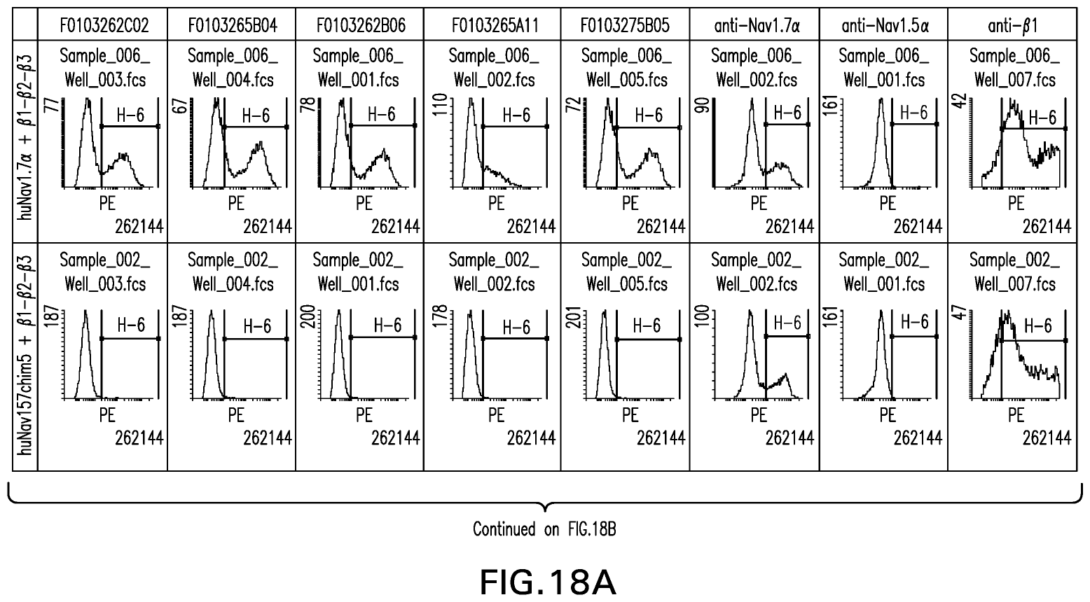

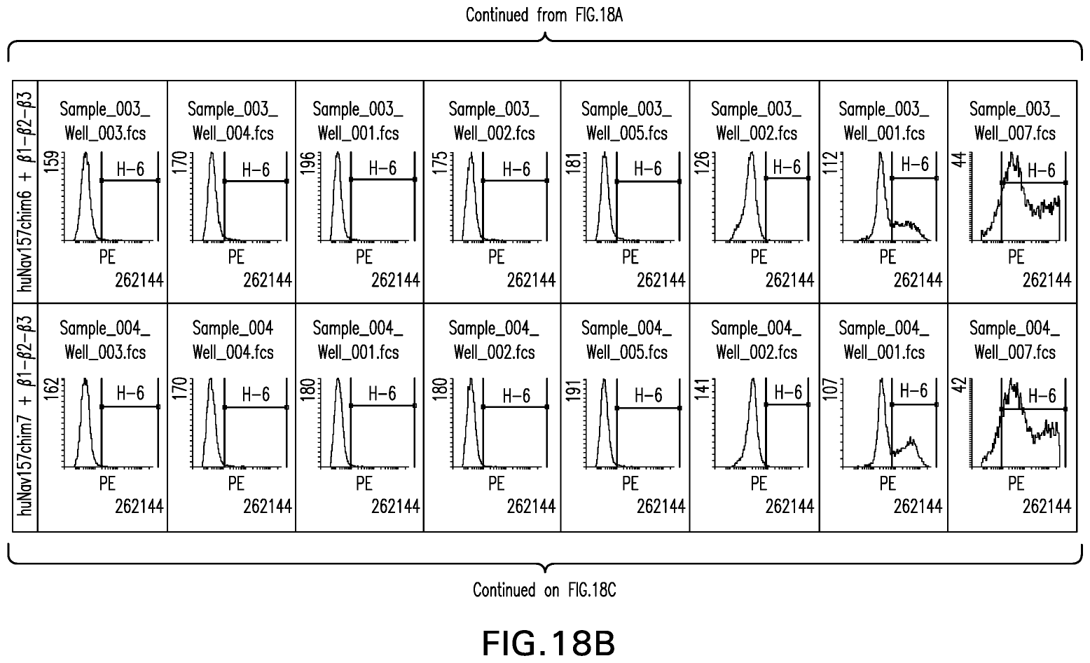

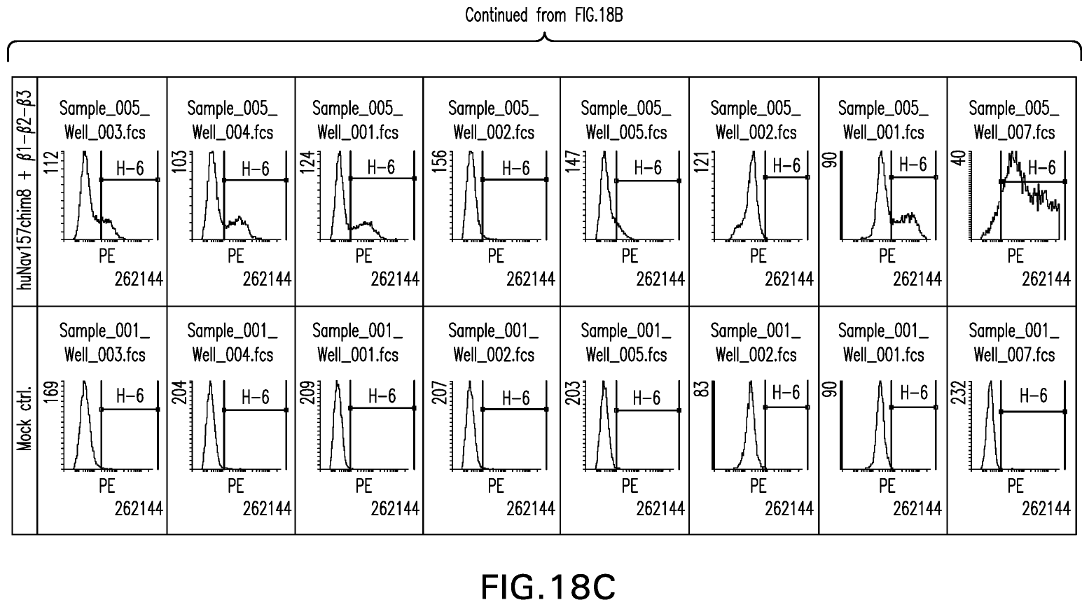

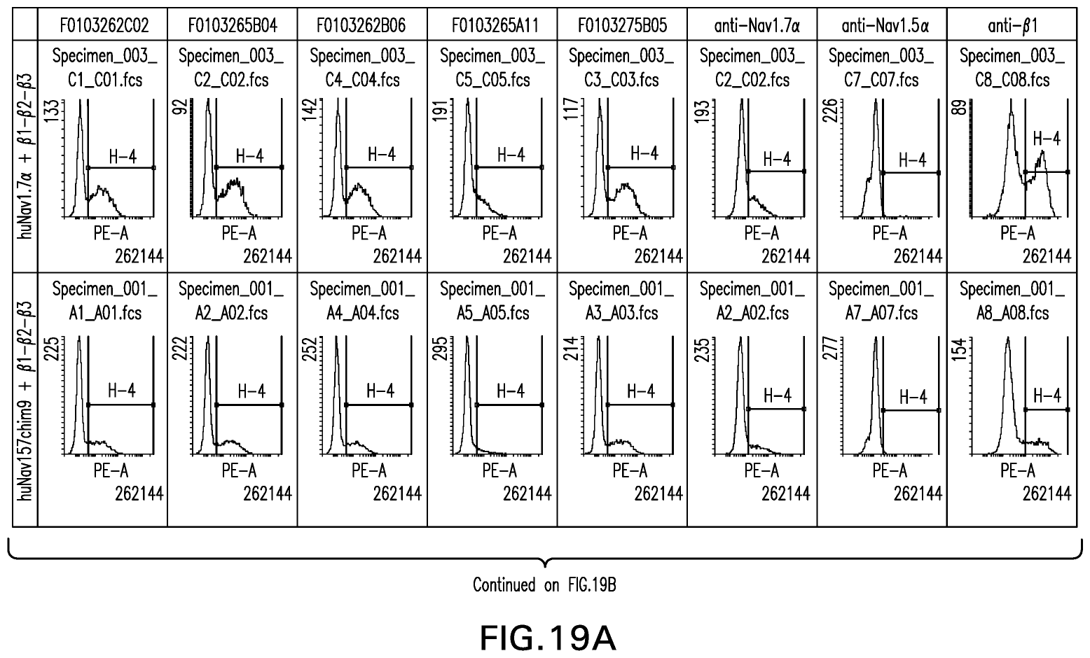

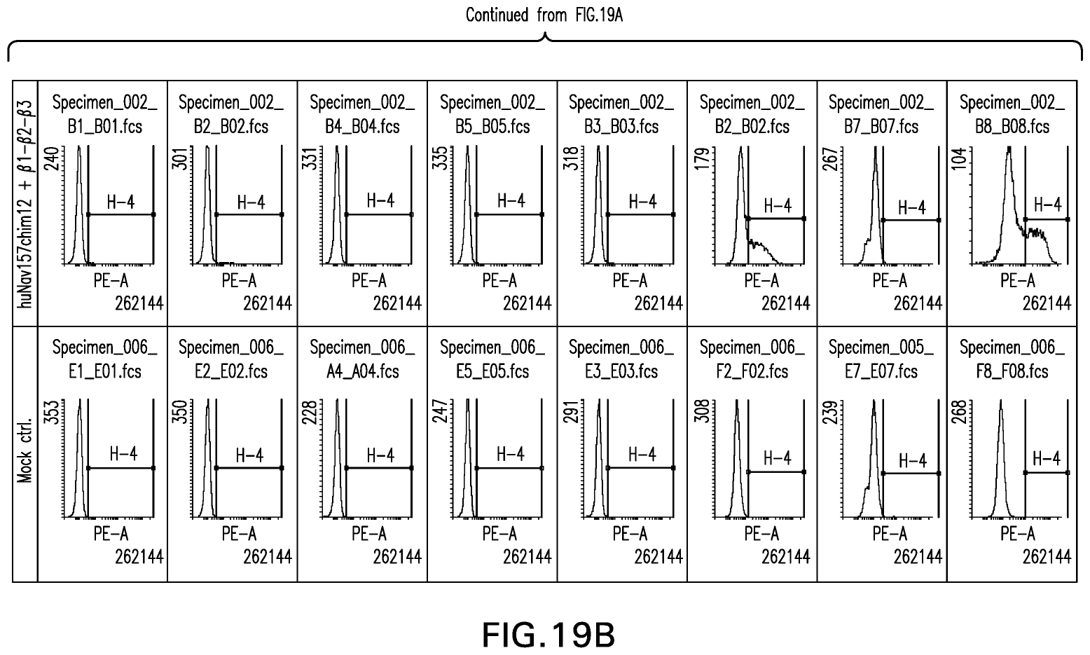

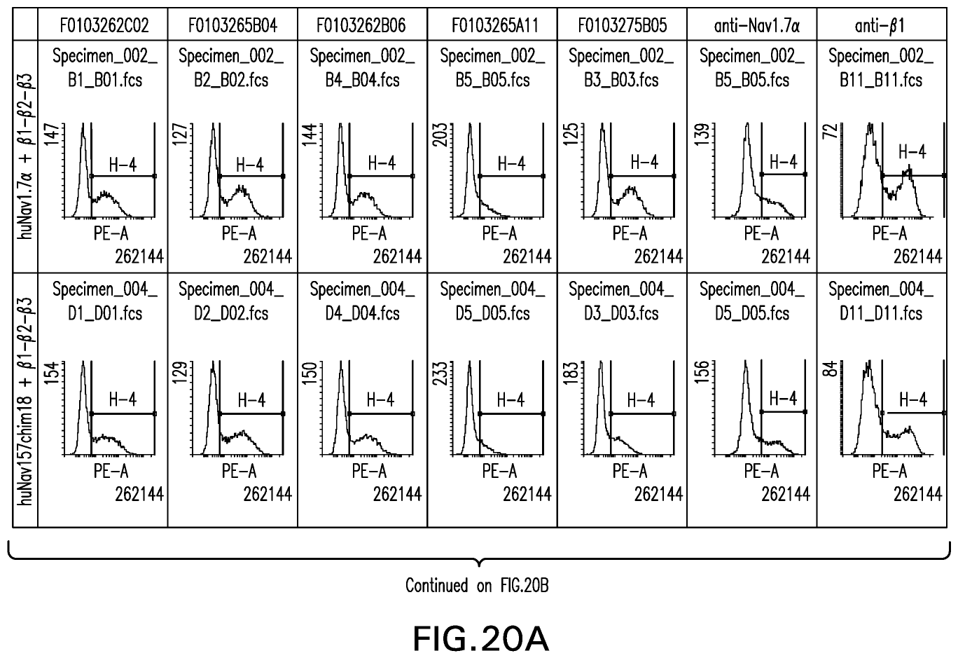

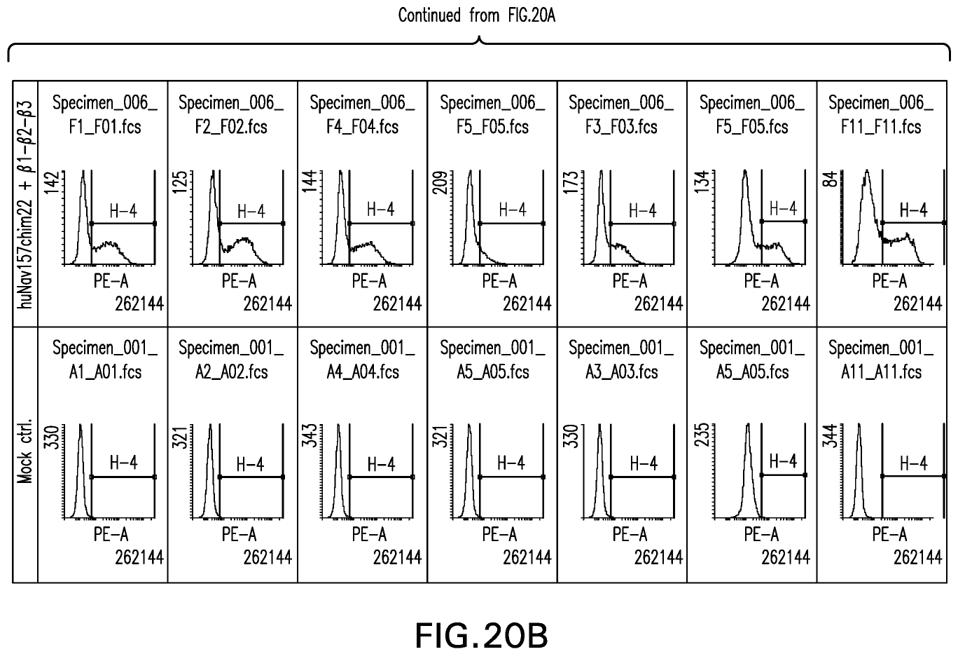

From experiments summarized in Table 22 and shown in FIG. 17A-FIG. 17C and FIG. 18A-18C, it could be concluded that DI of huNav1.7α is necessary and sufficient for the binding of F0103262CO2, F0103262B06, F0103265B04, F0103275B05, and F0103265A11 (see FIG. 16). From a separate experiment (Table 22 and FIGS. 19A-19B) with a chimeric variant of huNav1.7α in which the DI S5-S6 sequence is replaced by the huNav1.5 counterpart (chimera 12 in FIG. 16), it could be concluded that DI S5-S6 is necessary for the binding of F0103262CO2, F0103262B06, F0103265B04, F0103275B05, and F0103265A11 to huNav1.7α. In addition, F0103275B05 appears to also interact with the adjoining DIV VSD (Table 22, FIGS. 17A-17C, FIGS. 18A-18C, and FIGS. 20A-20B). Control antibodies murine anti-Nav1.7α mAb S68-6 (Abcam, catalog #ab85015) and rabbit anti-Nav1.5α pAb (Alomone Labs, catalog #ASC-013) recognize an epitope at the intracellular C-terminal part of their respective channel.

TABLE 22

| | SEQ | | | | | | | | | | |
|---|---|---|---|---|---|---|---|---|---|---|---|
| | ID | DI | | | DII | | | DIII | | | DIV |
| ID # | NO: | S1-S2 | S3-S4 | S5-S6 | S1-S2 | S3-S4 | S5-S6 | S1-S2 | S3-S4 | S5-S6 | S1-S2 |
| huNav1.7α + β1- | 3 | | | | | | | | | | |
| β2-β3 | | | | | | | | | | | |
| huNav157chim1 + | 10 | | | | | | | | | | X |
| β1-β2-β3 | | | | | | | | | | | |
| huNav157chim2 + | 11 | | | | | | | X | X | X | |
| β1-β2-β3 | | | | | | | | | | | |
| huNav157chim3 + | 12 | | | | X | X | X | | | | |
| β1-β2-β3 | | | | | | | | | | | |
| huNav157chim4+ | 13 | X | X | X | | | | | | | |
| β1-β2-β3 | | | | | | | | | | | |
| huNav157chim5+ | 14 | X | X | X | X | X | X | X | X | X | |
| β1-β2-β3 | | | | | | | | | | | |
| huNav157chim6+ | 15 | X | X | X | X | X | X | | | | X |
| β1-β2-β3 | | | | | | | | | | | |
| huNav157chim7+ | 16 | X | X | X | | | | X | X | X | X |
| β1-β2-β3 | | | | | | | | | | | |
| huNav157chim8+ | 17 | | | | X | X | X | X | X | X | X |
| β1-β2-β3 | | | | | | | | | | | |
| huNav157chim9+ | 18 | | | | | | | | | | |
| β1-β2-β3 | | | | | | | | | | | |
| huNav157chim12 | 19 | | | X | | | | | | | |
| +β1-β2-β3 | | | | | | | | | | | |
| huNav157chim18 | 29 | | | | | | | | | | X |
| +β1-β2-β3 | | | | | | | | | | | |
| huNav157chim22 | 21 | | | | | | | | | | X |
| +β1-β2-β3 | | | | | | | | | | | |

| | DIV | | | | | | |
|---|---|---|---|---|---|---|---|
| ID # | S3-S4 | S5-S6 | F0103262C02 | F0103275B05 | F0103265B04 | F0103262B06 | F0103265A11 |
| huNav1.7α + β1- | | | + | + | + | + | ± |
| β2-β3 | | | | | | | |
| huNav157chim1 + | X | X | + | − | + | + | ± |
| β1-β2-β3 | | | | | | | |
| huNav157chim2 + | | | + | + | + | + | ± |
| β1-β2-β3 | | | | | | | |
| huNav157chim3 + | | | + | + | + | + | ± |
| β1-β2-β3 | | | | | | | |
| huNav157chim4+ | | | − | − | − | − | − |
| β1-β2-β3 | | | | | | | |
| huNav157chim5+ | | | − | − | − | − | − |
| β1-β2-β3 | | | | | | | |
| huNav157chim6+ | X | X | − | − | − | − | − |
| β1-β2-β3 | | | | | | | |
| huNav157chim7+ | X | X | − | − | − | − | − |
| β1-β2-β3 | | | | | | | |
| huNav157chim8+ | X | X | + | ± | + | + | ± |
| β1-β2-β3 | | | | | | | |
| huNav157chim9+ | | X | + | + | + | + | ± |
| β1-β2-β3 | | | | | | | |
| huNav157chim12 | | | − | − | − | − | − |
| +β1-β2-β3 | | | | | | | |
| huNav157chim18 | X | | + | ± | + | + | ± |
| +β1-β2-β3 | | | | | | | |
| huNav157chim22 | | | + | ± | + | + | ± |
| +β1-β2-β3 | | | | | | | |

X boxes, Nav1.5;
empty boxes, Nav1.7α;
+, strong binding;
± weak binding;
−, no binding

### Example 6

Binding to huNav1.7α-rhNav1.7α Chimeras

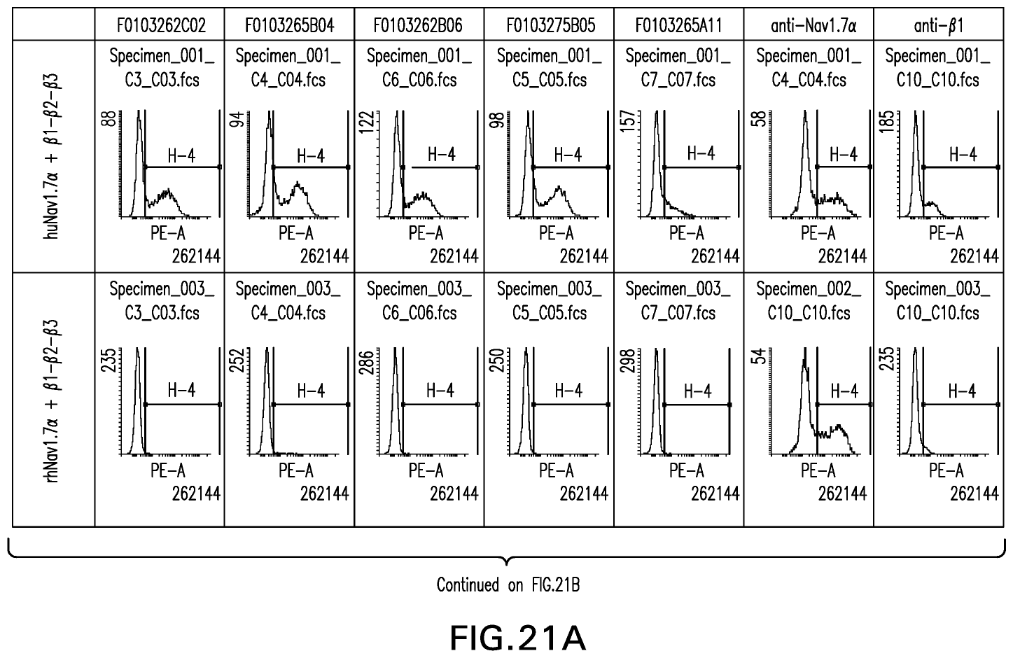

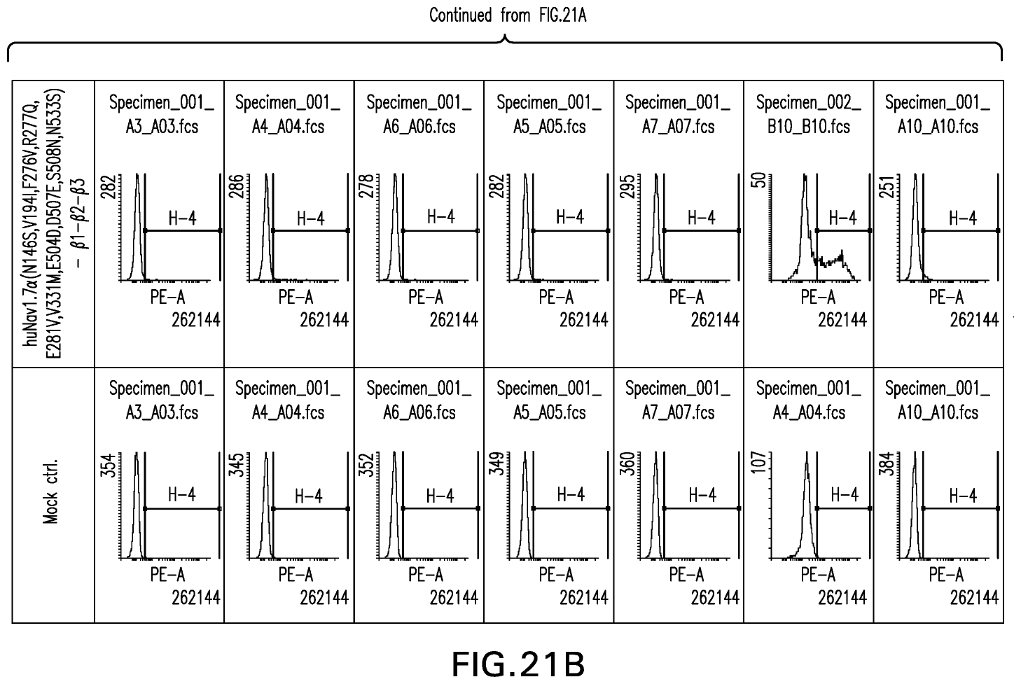

FACS binding studies (as described above) were performed on HEK293T cells transiently transfected with a chimeric variant of huNav1.7α in which all the huNav1.7α-rhNav1.7α polymorphisms of DI are present (N146S, V1941, F276V, R277Q, E281V, V331M, E504D, D507E, S508N, N533S). Replacing the huNav1.7α DI sequence for that of rhNav1.7α is sufficient to abolish the binding of F0103262CO2, F0103265B04, F0103262B06, and F0103265A11 to huNav1.7α, recapitulating the absence of binding on rhNav1.7α (see FIGS. 21A-21B).

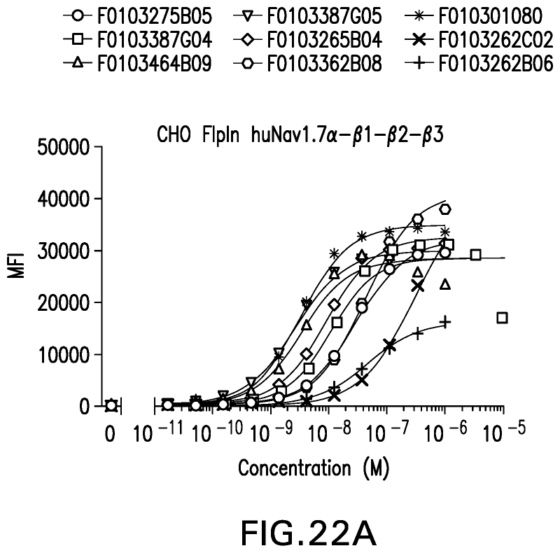

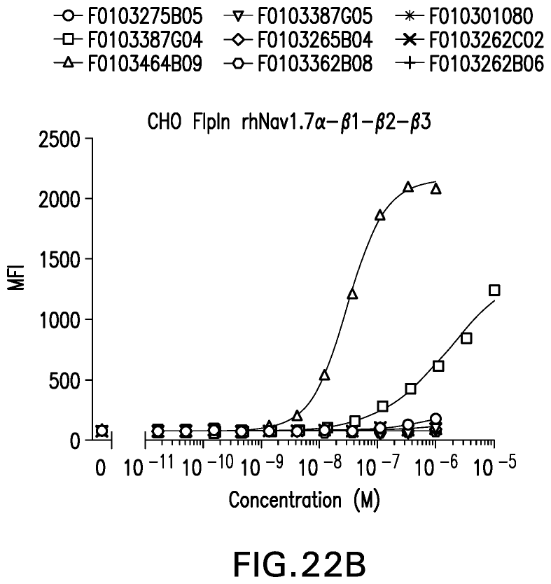

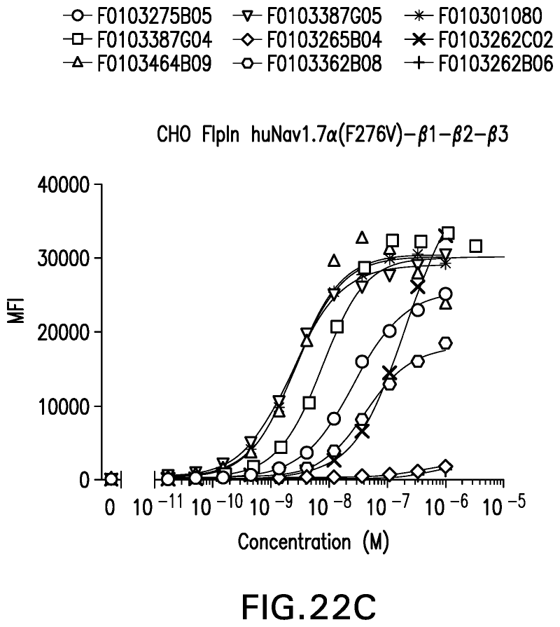

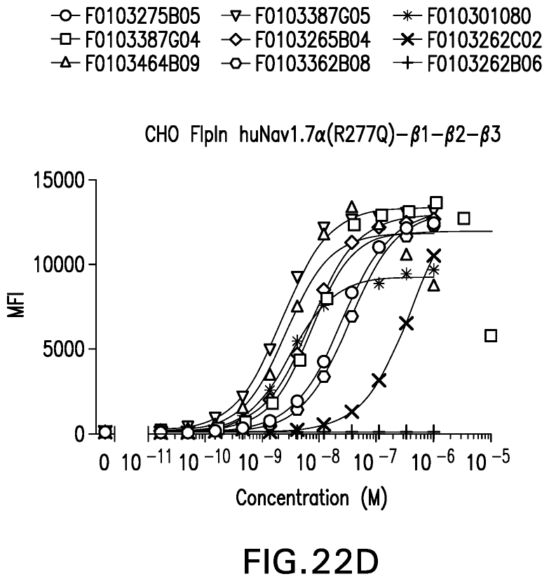

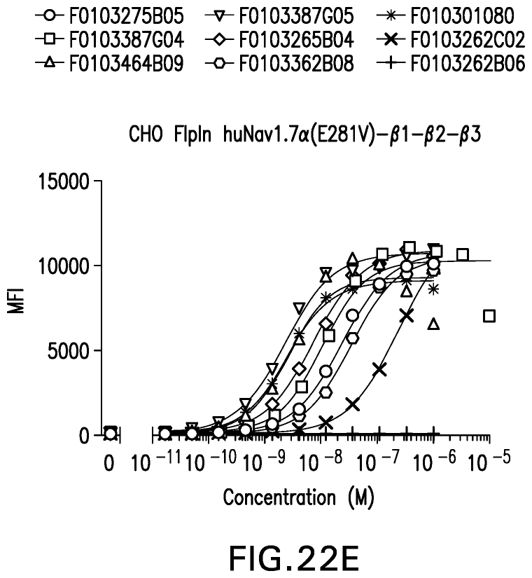

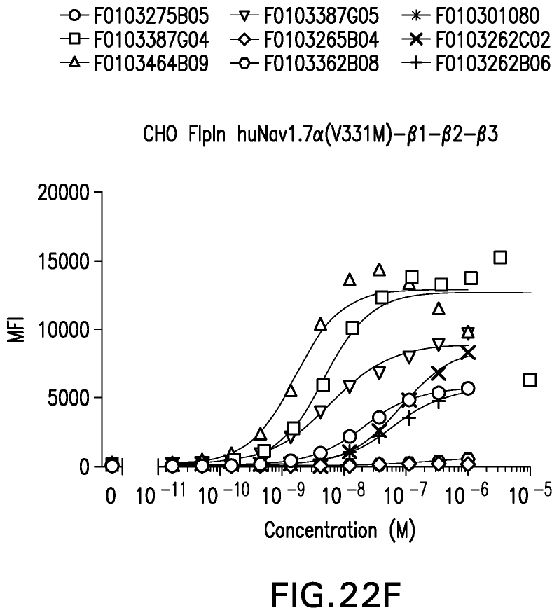

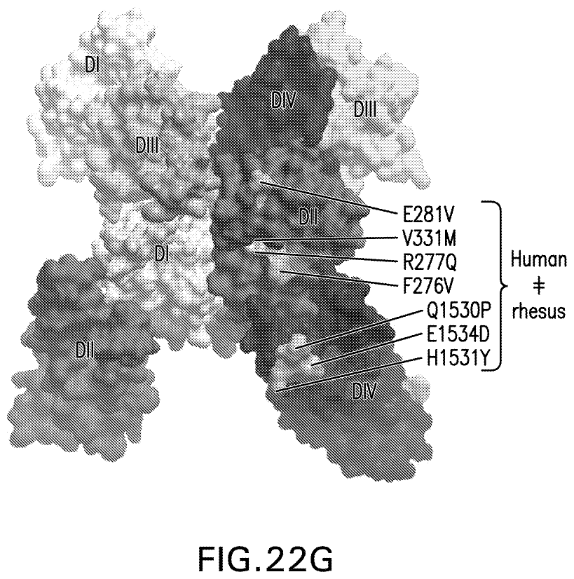

Based on the huNav1.7α model (as described above) the following huNav1.7α-rhNav1.7α polymorphisms can be allocated to the extracellular part of DI: N146S, F276V, R277Q, E281V and V331M. The first of the residues is in DI S1-S2 whereas the latter four residues belong to DI S5-S6. FACS binding studies (as described above) were performed to stable CHO FlpIn cell lines expressing different variants of huNav1.7+β1−β2−β3 each including one of the four possible extracellular huNav1.7α-rhNav1.7α polymorphisms in the DI S5-S6 region: F276V, R277Q, E281V and V331M (Table 23 and FIGS. 22A-22F). Individual polymorphisms were each sufficient to abolish the binding of F0103265B04, F0103362B08, F010301080, and F0103262B06 to huNav1.7α, recapitulating the absence of binding on rhNav1.7α. F0103262CO2, F0103275B05 and F0103387G05 are more subtly affected in terms of EC50 or Bmax by some of the polymorphisms. None of the individual DI S5-S6 polymorphisms by themselves appear to have an impact on the binding of the two rhNav1.7α cross-reactive ISVDs F0103387G04 and F0103464B09. In addition, no effect on binding of the two ISVDs was observed (data not shown) for the three extracellular DIV VSD huNav1.7α-rhNav1.7α polymorphisms Q1530P, H1531Y and E1534D (FIG. 22G).

TABLE 23

| | | CHO | CHO | CHO | CHO | |
|---|---|---|---|---|---|---|
| | CHO | FlpIn | FlpIn | FlpIn | FlpIn | CHO |
| | FlpIn | huNav1.7α | huNav1.7α | huNav1.7α | huNav1.7α | FlpIn |
| | huNav1.7α + | (F276V) + | (R277Q) + | (E281V) + | (V331M) + | rhNav1.7α + |
| | β1-β2- | β1-β2-β3 | β1-β2-β3 | β1-β2-β3 | β1-β2- | β1-β2- |
| | β3 (SEQ | (SEQ ID | (SEQ ID | (SEQ ID | β3 (SEQ | β3 (SEQ |
| | ID NO: 3) | NO: 5) | NO: 6) | NO: 7) | ID NO: 8) | ID NO: 4) |
| ID # | EC50 [M] | EC50 [M] | EC50 [M] | EC50 [M] | EC50 [M] | EC50 [M] |
| F0103265B04 | 9.2E−09 | — | 7.1E−09 | 7.5E−09 | — | — |
| F0103362B08 | 4.3E−08 | 4.2E−08 | 3.4E−08 | 3.4E−08 | — | — |
| F0103262C02 | 2.9E−07 | 2.1E−07 | 4.5E−07 | 2.4E−07 | 8.6E−08 | — |
| F0103262B06 | 4.7E−08 | — | — | — | 6.3E−08 | — |
| F010301080* | 3.4E−09 | 2.6E−09 | 3.2E−09 | 2.5E−09 | — | — |
| F0103275B05 | 3.0E−08 | 2.6E−08 | 2.5E−08 | 2.4E−08 | 2.1E−08 | — |
| F0103387G04 | 1.1E−08 | 7.4E−09 | 7.1E−09 | 1.1E−08 | 4.9E−09 | 5.9E−07 |
| F0103387G05 | 2.6E−09 | 2.4E−09 | 2.2E−09 | 2.3E−09 | 5.9E−09 | — |
| F0103464B09 | 3.4E−09 | 2.7E−09 | 2.6E−09 | 2.7E−09 | 1.6E−09 | 3.1E−08 |

—, no binding;
*F0103265A11(S53W, T57V, N58T, V60N) affinity maturation variant

### Summary Epitope Mapping

The combined data of the binding studies on the huNav157 chimeras and the huNav1.7α-rhNav1.7α chimeras, together with the competition binding data suggests that the ISVDs recognize an overlapping epitope on the DI S5-S6 part of huNav1.7α, which can be further delineated by the extracellular human-rhesus polymorphisms in that part which can be further dissected out by the extracellular huNav1.7α-rhNav1.7α polymorphisms in that area or by additional contacts with the adjoining DIV VSD in the case of F0103275B05.

### Example 7

Electrophysiological characterization of Nav1.7α selective ISVDs on IonFlux 16 automated patch clamp system (Fluxion Biosciences, Inc., Alameda, CA).

### Solutions and ISVDs Handling

The extracellular solution contained (in mM): 138 NaCl, 4 KCl, 1.8 CaCl₂, 1 MgCl₂, 10 HEPES, 5.6 glucose (pH 7.2 with NaOH, and 285-290 mOsmolar). Intracellular solution contained (in mM): 5 NaCl, 100 CsF, 45 CsCl, 10 HEPES, 5 EGTA (pH 7.45 with CsOH, and 300-315 mOsmolar). These solutions were freshly made, filtered and stocked for no longer than 6 months at 4° C.

### Cell Preparation

HEK Flp-In and CHO Flp-In cells stably expressing the human Nav1.7α channel were generated. Cells were cultured in T-175 cell culture flasks (Greinerbio-one, catalog #660160) using standard cell culture conditions. CHO Flp-In culture medium consists of F12 nutrient mix (Gibco, catalog #31765) containing 10% FBS (Sigma-Aldrich, catalog #F7524), 0.8 mg/mL hygromycin B (Invitrogen, catalog #10687010). HEK Flp-In culture medium consists of DMEM Glutamax™ (Gibco, catalog #31966) containing 10% FBS (Sigma-Aldrich, catalog #F7524), 0.8 mg/mL hygromycin B (Invitrogen, catalog #10687010), 1% NEAA (Gibco, catalog #11140) and 1% Na-pyruvate (Gibco, catalog #11360). Cells were seeded at a density of 1.7×10 4 cells/cm 2 (Hek293 Flp-In) or 5.7×10 3 cells/cm 2 (CHO Flp-In) for 2 days before being used in the IonFlux 16 (Fluxion). Optimal cell confluence prior to harvesting never exceeded 80%. The cells were washed twice with d-PBS without Ca²⁺ and Mg²⁺(Gibco, catalog #14190) and detached with 4 mL Trypsin/EDTA 0.25% (Invitrogen, catalog #25200-056) for 5 to 10 min at 37° C. Medium containing 10% FBS is added to inactivate the enzymatic reaction triggered by the trypsin. Subsequently, the cells were counted (Casy TT, Roche) and centrifuged at 200×g during 2 min at RT in 50 mL conical CELLSTAR® tube (Greiner Bio-One, catalog #227-261) suspended at 1×10 6 cells/ml in CHO—S-SFMII (Gibco, catalog #12052) supplemented with 25 mM Hepes (Gibco, catalog #15630), transferred to a 25 mL cell culture flask (Greiner Bio-One, catalog #690190) and gently shaken at RT for approximately 20 min. 1×10 7 cells were centrifuged for 2 min at 200×g. The pellet is gently resuspended in 5 mL extracellular buffer and centrifuged a second time for 2 min at 200×g. Finally, the pellet is resuspended in 2000 μl extracellular buffer and immediately tested on the IonFlux.

### IonFlux Automated Patch Clamp Procedure

250 μL of sterile cell culture grade water is dispensed into every well of the IonFlux 96-well plate except the outlet wells, using an eight channel multi-pipette. Any excess water on the rim of the plate is wiped off before rinsing the plate. The designated plate is inserted into the IonFlux system and subsequently rinsed 4 times according to a standard Water Rinse protocol. After rinsing, the plate is emptied. The inlet wells were then manually filled with extracellular buffer, trap wells with intracellular buffer and the diluted ISVDs or selective peptides were distributed into the compounds wells (250 μL/well). Subsequently, the plate is primed before the actual experiment according to the plate specific protocols. For population plates (Molecular Devices, catalog #910-0098): 1) traps and compounds at 5 psi for t=0-160 s and 2 psi for t=160-175 s, 2) traps but not compounds at two psi for t=175-180 s, and 3) main channel at 1 psi for t=0-160 s and 0.3 psi for t=162-180 s. For single cell plates (Molecular Devices, #910-0100): 1) traps but not compounds at eleven psi for t=0-350 s and 1.5 psi for t=625-630 2) traps and compounds at five psi for t=350-600 s and 1.5 psi for t=600-625 s, and 3) main channel at 0.5 psi for t=0-350 s and one psi for t=350-600 s, and 0.3 psi for t=600-627 s. After priming, the outlet and inlet wells were emptied and 250 μL of the prepared cell suspension (i.e. approximately one million cells) is distributed into the inlet wells of the designated plate. After introduction of the cells, the plate is reprimed: 1) traps and compounds at five psi for t=0-20 seconds and two psi for t=25-50 seconds, 2) traps not with compounds at two psi for t=50-55 seconds, and 3) main channel at one for t=0-30 seconds and 0.4 psi for t=30-55 seconds. Then, cells were introduced to the main channel and trapped at lateral trapping sites with the trapping protocol: 1) trapping vacuum of 7 mmHg for t=0 to 85 seconds, 2) main channel pressure of 0.2 psi for t=0-2 seconds, followed by 15 repeated square pulses of 0-0.2 psi with baseline duration of 4.2 seconds and pulse duration of 0.8 seconds, followed by 0.2 psi for 8 seconds. Whole cell access is achieved by rupturing the patch of the membrane over the hole using the break protocol. A different protocol is used for CHO or HEK293 cells. Breaking protocol for HEK293 cells: 1) breaking vacuum of seven mmHg for t=0-5 seconds, followed by a square pulse of 18 mmHg with a pulse duration of 15 seconds, and followed by 6 mmHg for five seconds, and 2) main channel pressure at 0.15 psi for t=0-25 seconds. Breaking protocol for CHO cells: 1) breaking vacuum of seven mmHg for t=0-10 seconds, followed by a square pulse of 25 mmHg with a pulse duration of five seconds, followed by 6 mmHg for 6 seconds, and a second pulse of 25 mmHg with a pulse duration of five seconds, followed by 6 mmHg for 80 seconds, and 2) main channel pressure at 0.15 psi for t=0-120 seconds. After whole cell configuration, the vacuum pressure is held at 5 mmHg and the main channel pressure at 0.1 psi until the end of the experiment. Cells were first allowed to dialyze for 300 seconds, before compounds were tested. A time course protocol is applied to assess the effect of the compounds on sodium currents elicited by a depolarizing pulse protocol. In order to be able to perform an off-line linear leak subtraction, cells were clamped at −100 mV for 50 milliseconds then hyperpolarized to −120 mV for 100 milliseconds, and repolarized to −80 mV for 30 milliseconds.

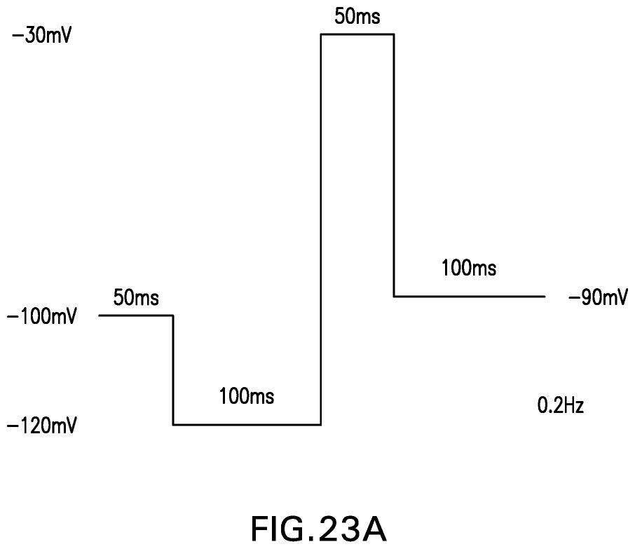

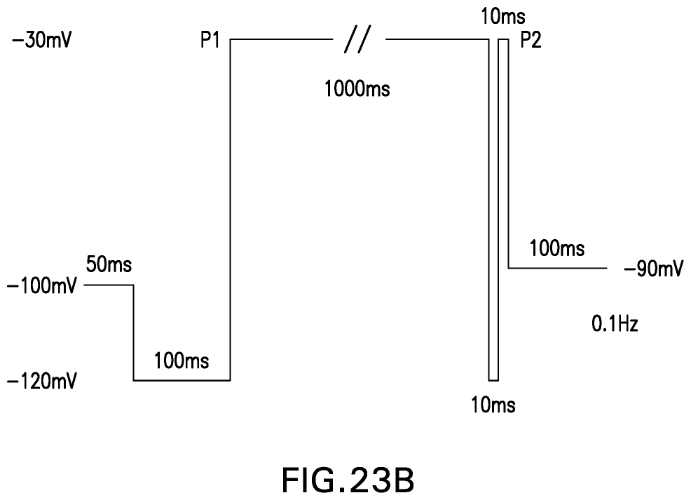

Two data acquisition protocols were used: single pulse and two pulse. Single pulse protocol: cells were clamped at a holding potential of −100 mV, stepped to −120 mV for 100 milliseconds to maximize channel availability and then to −30 mV for 50 milliseconds to open the Nat channels. The sweep interval was five seconds with a holding potential of −80 Mv (FIG. 23A). For the two pulse protocol sodium currents were elicited by a depolarizing step from −80 mV to −30 mV for 1000 millieseconds, followed by 10 ms hyperpolarization at −120 mV and a second depolarizing step at −30 mV for 10 milliseconds. The sweep interval was 9 seconds with a holding potential of −80 mV (FIG. 23B).

After the stabilizing period, extracellular buffer is continuously perfused for 120 seconds as a negative control, followed by sequential perfusion of different concentrations of ISVDs or selective peptides. The inhibitory responses were recorded at room temperature (21° C.-24° C.) with a minimum of n=2 for each compound.

### IonFlux Data Inclusion Criteria and Data Analysis

Data points were accepted when:

### (A) Automated Population Patch

a. Individual membrane resistance quality and stability is greater than 3 MS2 during data acquisition
b. Current amplitude quality and stability is greater than 2 nA at −30 mV after negative control
c. Run-up/run-down less than 10% during data acquisition
d. IC₅₀ value for reference compounds within anticipated range
   (B) Automated Single cell patch
a. Individual membrane resistance quality and stability is greater than 500 MΩ during data acquisition
b. Current amplitude quality and stability is greater than 200 pA at −30 mV after negative control
c. Run-up/run-down less than 10% during data acquisition
d. IC50 value for reference compounds within anticipated range

Currents were measured using IonFlux software (Fluxion Biosciences) and monitored continuously during the exposure to the compounds. Measured currents were normalized by the mean I sustained corrected amplitude prior to compound addition. Current inhibition is estimated by the residual response after 120 seconds of each compound application. Data analysis was performed with IonFlux software (Fluxion Biosciences), Microsoft Excel (Microsoft) and Prism 6 (GraphPad Software).

### Electrophysiology Experiments

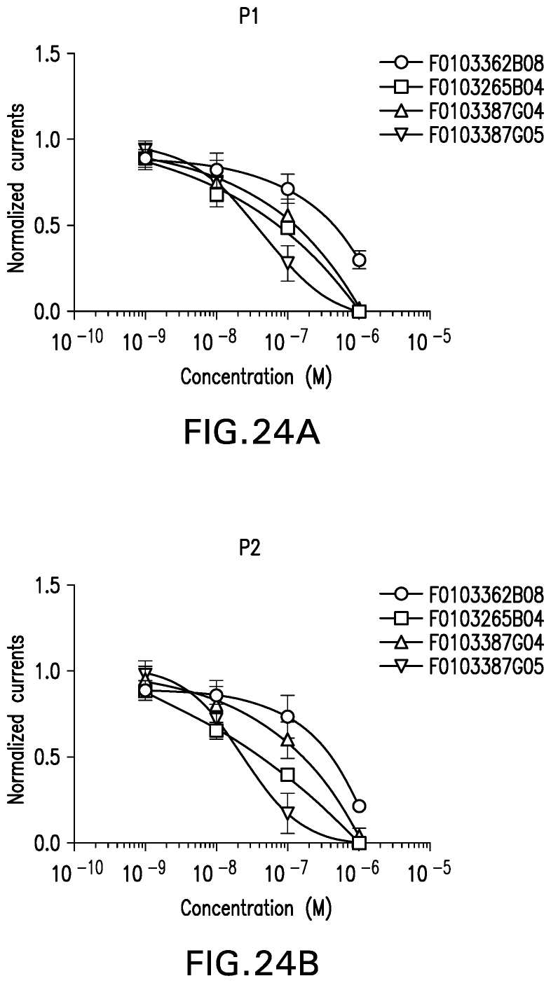

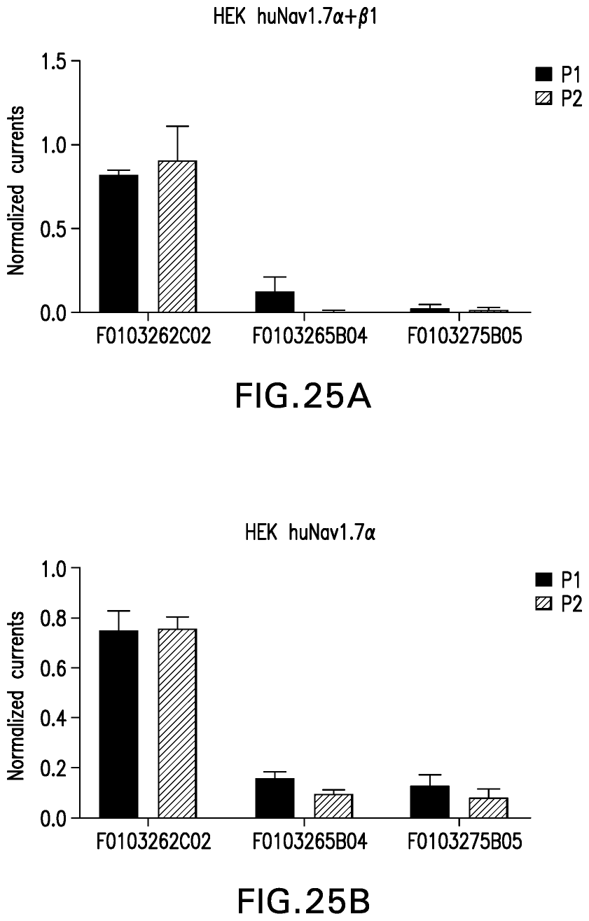

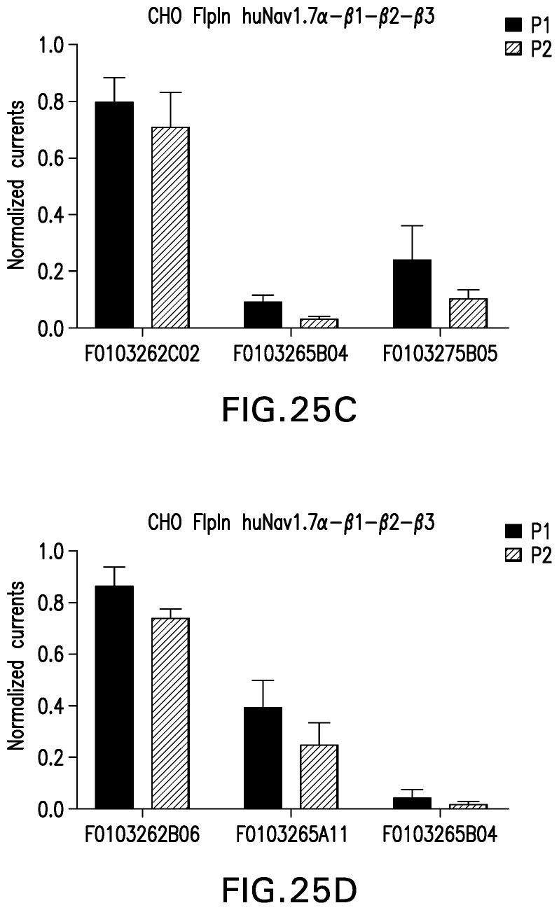

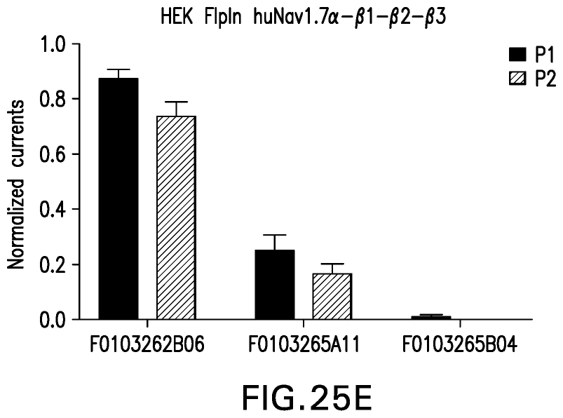

A series of experiments was performed, using the two pulse protocol shown in FIG. 23B and a single concentration (1 μM) of F0103265B04, F0103265A11, F0103275B05, F0103362B08, F0103387G04, F0103387G05, F0103262CO2, and F0103262B06 applied for 5 minutes to CHO Flpin huNav1.7α+Navβ1-βNav2-Navβ3 cells, HEK293 huNav1.7α+Navβ1 cells, HEK293 huNav1.7α cells and HEK FlpIn huNav1.7α+Navβ1-Navβ2-Navβ3 cells (see FIGS. 25A-25E). In another experiment, a concentration range (1 μM to 1 nM) of F0103265B04 and F0103362B08 was applied to HEK Flpin huNav1.7α+Navβ1-Navβ2-Navβ3 cells, using the same protocol (see FIG. 24). From these experiments it could be concluded that F0103265B04, F0103265A11, F0103275B05, F0103362B08, F0103387G04 and F0103387G05 but not F0103262CO2 or F0103262B06, functionally inhibit huNav1.7α currents in a dose-dependent manner.

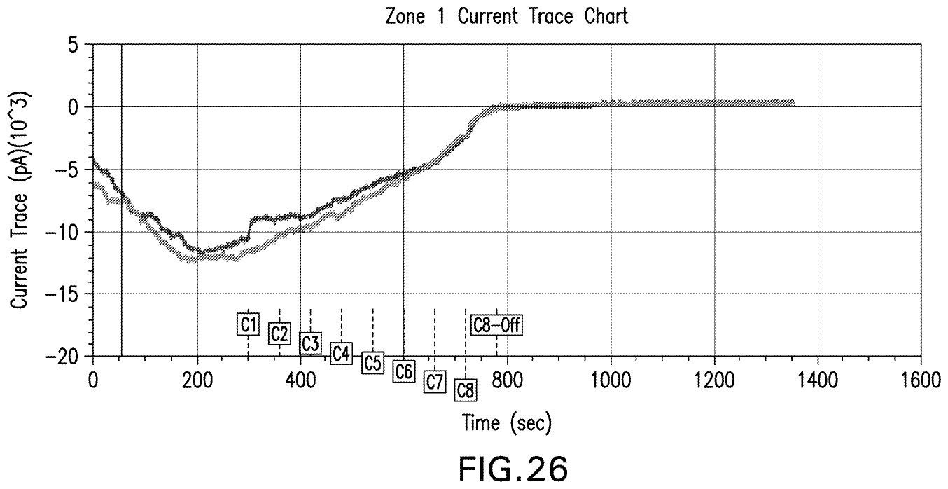

After the application of F0103265B04 to the cells was stopped and the compound was allowed to wash out by application of buffer, the cells were continued to be monitored on the patch clamp. The inhibitory effect of F0103265B04 did not wash out in the time frame (11 minutes) of the experiment (see FIG. 26).

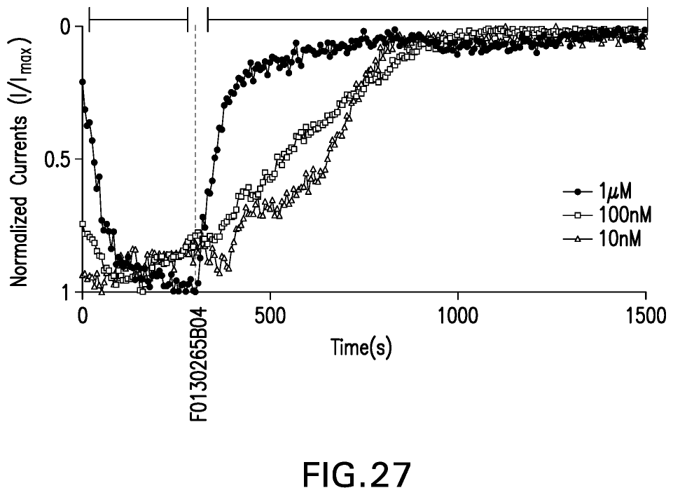

A time course experiment with F0103265B04 using the single pulse protocol (see FIG. 25A) revealed that it takes greater than two minutes at 1 μM and greater than eight minutes for F0103265B04 at 10 nM and 100 nM to fully block of the huNav1.7α currents (see FIG. 27).

### Example 8

Sequence optimization is a process in which parental ISVD sequences are mutated to yield ISVD sequences that are more identical to human and/or llama/alpaca IGHV3-IGHJ germline consensus sequences. Specific amino acids, with the exception of the so-called hallmark residues, in the FRs that differ between the ISVD and the human IGHV3-IGHJ germline consensus are altered to the human counterpart in such a way that the protein structure, activity and stability are kept intact. In addition, the amino acids present in the CDRs for which there is experimental evidence that they are sensitive to post-translational modifications (PTMs) are altered in such a way that the PTM site is inactivated while the protein structure, activity and stability are kept intact. Furthermore, in order to reduce the binding of pre-existing antibodies to the ISVDs, certain FR residues are altered.

Amino acid residue differences in the CDR regions are not taken into account for sequence optimization. All amino acid differences in the FRs between the ISVD and the human VH341-1 consensus counterparts are identified. Typically, these amino acid residues (numbered according to Kabat) fall into three classes:

1. Hallmarks: These residues are known to be critical for the stability/activity/affinity of the ISVD (based on literature). Therefore, these positions are usually not included in the process. Only when a hallmark is deviating from its llama germline, it is taken into account to be mutated back to the llama/alpaca germline sequence to evaluate potential improvements in stability/activity/affinity. When taken into account this mutation is investigated on an individual basis.
2. Standard: Sequence optimization of these positions is not expected to dramatically change the stability/activity/affinity of the ISVD (based on previous sequence optimization efforts) and they are therefore altered all at once, yielding a basic variant.
3. Unique: It is not known if sequence optimization of these positions affects the stability/activity/affinity of the ISVD and therefore they are investigated on an individual basis on top of the basic variant. These positions typically differ from ISVD to ISVD.

A potential PTM site will only be mutated when there is evidence that the particular site is sensitive to modification under accelerated stress conditions. If a particular amino acid position is insensitive, the parental sequence will be left unchanged in the final construct. Assessment of chemical stability by means of accelerated stress studies is performed by CMC. The N-terminal Glu residue of the first block of an ISVD construct will always be mutated to an Asp (E1D) because experimental evidence has shown that the majority of ISVDs is significantly sensitive to pyroglutamate formation and that the E1D mutation has no effect on stability/activity/affinity of the ISVD. The E1 residues of all other building blocks in the construct are not mutated.

In order to reduce the binding of pre-existing antibodies to the ISVDs, L11V and V89L substitutions are introduced to the FRs and an Ala residue is added to the very C-terminus of the ISVD construct. Exceptionally, the T110K mutation may be introduced as well. The “humanness” of a sequence optimized ISVD may be defined as:

Percent amino acid identity in the FRs of the ISVD vs the human VH3-JH consensus sequence
wherein the CDRs may be defined by Kabat, IMGT, AbM, Chothia, or the like. In particular embodiments, the calculation is performed in which the CDRs are defined by at least two methods.

### Sequence Optimization of F0103275B05/F0103387G04

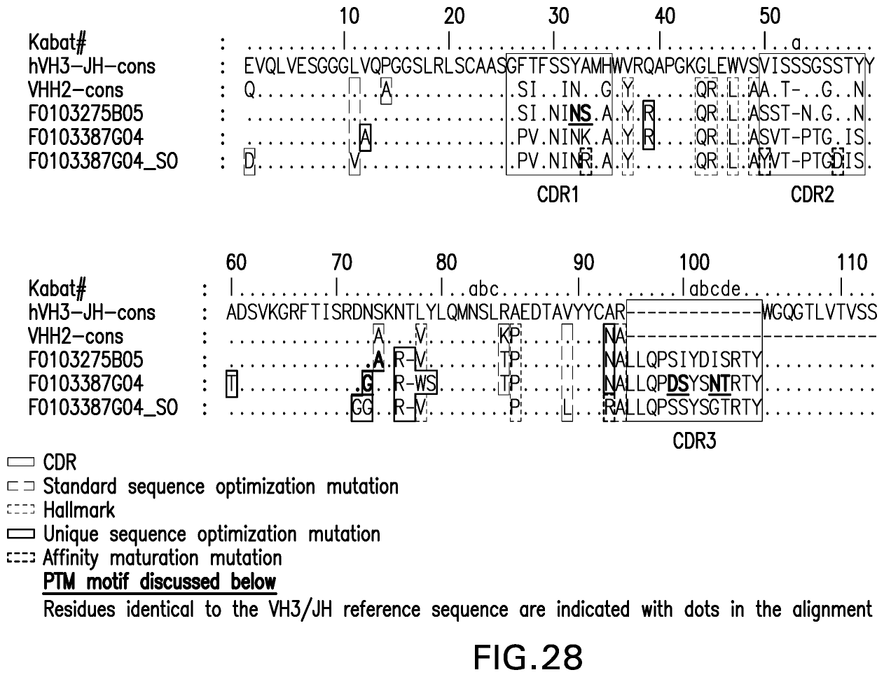

Several PTM substitution libraries were generated based on the accelerated stress data summarized in Table 24 and screened as crude periplasmic extracts in binding FACS on human and rhesus Nav1.7α. FIG. 28 shows a sequence analysis of F0103275B05/387G04 aligned against the human VH3-J3 consensus sequence and the llama VHH2 consensus sequence.

TABLE 24
Results accelerated stress experiments performed on F0103275B05/387G04 variants

| | | | Stress | Modification |
|---|---|---|---|---|
| ID # | Description | Site | condition | observed |
| F010300659 | F0103275B05(S27P, I28V, | NA | 1 week @, 45° | 0.2% increase |
| | S50Y, N53P, G55W, S56D, | | C., ±1 mg/mL | of pre-peak |
| | T57W, N93R, A94W) + | | in D-PBS | (SE-HPLC) |
| | FLAG3-HIS6 | | | |
| F010301452 | F0103275B05(S27P, I28V, | NA | 1 week @ 45° | 0.1% increase |
| | S50Y, N53P, G55W, S56D, | | C., ±1 mg/mL | of pre-peak |
| | T57W, N93R, A94W) | | in D-PBS | (SE-HPLC) |
| F010301457 | F0103275B05(S27P, I28V, | N32 | 4 weeks @ −20, | Not sensitive |
| | S50Y, N53P, G55W, S56D, | | 25 and 40° C. | |
| | T57W, N93R, A94W) + HIS6 | | | |
| F010301457 | F0103275B05(S27P, I28V, | N73 | 4 weeks @, −20, | 57% |
| | S50Y, N53P, G55W, S56D, | | 25 and 40° C. | |
| | T57W, N93R, A94W) + HIS6 | | | |
| F010301894 | F0103387G04(L11V, A12V, | D72 | 4 weeks @ −20, | 1.8%-12.3% |
| | K33R, R39Q, S50Y, S56D, | | 25 and 40° C. | |
| | T60A, R76N, W78V, S79Y, | | | |
| | T83R, V89L, N93R) + HIS6 | | | |
| F010301894 | F0103387G04(L11V, A12V, | N100c | 4 weeks @ −20, | 1.4-8.9% |
| | K33R, R39Q, S50Y, S56D, | | 25 and 40° C. | |
| | T60A, R76N, W78V, S79Y, | | | |
| | T83R, V89L, N93R) + HIS6 | | | |
| F010301895 | F0103387G04 + HIS6 | D72 | 4 weeks @ −20, | 0.1%-1.7% |
| | | | 25 and 40° C. | |
| F010301895 | F0103387G04 + HIS6 | N100c | 4 weeks @ −20, | 0.7-2.7% |
| | | | 25 and 40° C. | |
| F010301950 | F0103387G04(L11V, A12V, | M34 | 10 mM H₂O₂ | 5% |
| | K33R, R39Q, S50Y, S56D, | | for 3 h @ RT | |
| | T60A, W78V, S79Y, T83R, | | | |
| | V89L, N93R) + HIS6 | | | |
| F010301950 | F0103387G04(L11V, A12V, | D72 | 4 weeks @ −20, | 3-15% |
| | K33R, R39Q, S50Y, S56D, | | 25 and 40° C. | |
| | T60A, W78V, S79Y, T83R, | | | |
| | V89L, N93R) + HIS6 | | | |
| F010301950 | F0103387G04(L11V, A12V, | N100c | 4 weeks @ −20, | 1.2-8.1% |
| | K33R, R39Q, S50Y, S56D, | | 25 and 40° C. | |
| | T60A, W78V, S79Y, T83R, | | | |
| | V89L, N93R) + HIS6 | | | |
| F010301950 | F0103387G04(L11V, A12V, | D99 | 4 weeks @ −20, | 0.5-4.3% |
| | K33R, R39Q, S50Y, S56D, | | 25 and 40° C. | |
| | T60A, W78V, S79Y, T83R, | | | |
| | V89L, N93R) + HIS6 | | | |
| F010302383 | F0103387G04(L11V, A12V, | NA | 1 week @ 45° | 0.2% increase |
| | K33R, R39Q, S50Y, S56D, | | C., ±1 mg/mL | of pre-peak |
| | T60A, D72G, W78V, S79Y, | | in D-PBS | (SE- HPLC) |
| | T83R, V89L, N93R, D99S, | | | |
| | N100cG) + FLAG3-HIS6 | | | |

NA, not applicable

### Screening of F0103275B05/387G04 PTM Substitution Libraries

Several PTM substitution libraries were generated based on the accelerated stress data summarized in Table 24 and screened as crude periplasmic extracts in binding FACS on human and rhesus Nav1.7α.
N73G substitution resulted in a comparable or slightly improved binding profile compared to the parental reference F0103275B05 (Table 25) and was retained as this was also the naturally occurring residue on this position in F0103387G04 (FIG. 28).
Both D72G and D72Q substitutions resulted in a comparable or slightly improved binding profile compared to the parental reference (Table 27) and were further evaluated, as well G73A and G73R substitutions.
D99S, D99R and D99N substitutions resulted in a comparable or slightly improved binding profile compared to the parental reference (Table 26) and were further evaluated, as well as S100R and S100V substitutions; S99 is the naturally occurring residue on this position in F0103275B05 (FIG. 28).
N100cI and N100cG (Kabat position 100c) substitutions resulted in a comparable or slightly improved binding profile compared to the parental reference (Table 27) and were further evaluated; I100c is the naturally occurring residue on this position in F0103275B05 (FIG. 28)

TABLE 25
Summary screening N73X & A74X substitution libraries

| | CHO Flp-In | | CHO Flp-In | |
|---|---|---|---|---|
| | huNav1.7α + β1-β2-β3 | | rhNav1.7α + β1-β2-β3 | |
| | (SEQ ID NO: 3) | | (SEQ ID NO: 4) | |
| | Mean | SD | Mean | SD |
| Substitution | MFI | MFI | MFI | MFI |
| A74C | 1371 | 178 | 1380 | 148 |
| A74D | 8876 | 623 | 10933 | 506 |
| A74E | 6156 | 485 | 6767 | 677 |
| A74F | 11022 | 799 | 16436 | 605 |
| A74G | 12165 | 1360 | 18665 | 1369 |
| A74H | 12477 | 2828 | 18686 | 4367 |
| A74I | 4945 | 956 | 7560 | 1518 |
| A74K | 15407 | 1520 | 18490 | 1754 |
| A74L | 7173 | 1605 | 10190 | 1736 |
| A74N | 18129 | 463 | 26491 | 518 |
| A74P | 8928 | 2193 | 14486 | 3731 |
| A74Q | 9585 | 912 | 14644 | 1030 |
| A74R | 11403 | 43 | 16344 | 1503 |
| A74S | 10010 | 1672 | 15420 | 3795 |
| A74T | 9496 | 70 | 13521 | 666 |
| A74V | 6136 | 3294 | 9381 | 4939 |
| A74W | 9065 | 680 | 14913 | 922 |
| A74Y | 14759 | 324 | 21008 | 808 |
| Blanc | 515 | 37 | 482 | 35 |
| Parental reference | 10260 | 2842 | 15695 | 4311 |
| Negative control | 516 | 47 | 560 | 149 |
| N73A | 9387 | 2705 | 14029 | 3929 |
| N73C | 1396 | 8 | 1910 | 66 |
| N73E | 6536 | 478 | 9752 | 768 |
| N73F | 6341 | 714 | 9340 | 1556 |
| N73G | 18297 | 4375 | 27008 | 4900 |
| N73H | 10773 | 105 | 16937 | 830 |
| N73I | 4274 | 1138 | 6184 | 1751 |
| N73K | 13248 | 1558 | 17919 | 1854 |
| N73L | 4640 | 306 | 6839 | 199 |
| N73M | 5088 | 7 | 7638 | 112 |
| N73P | 4910 | 345 | 6851 | 610 |
| N73Q | 6614 | 1334 | 9716 | 2509 |
| N73R | 11583 | 1642 | 17083 | 2197 |
| N73S | 12569 | 2453 | 19307 | 3068 |
| N73T | 8839 | 279 | 14387 | 99 |
| N73V | 4827 | 185 | 7467 | 91 |
| N73W | 5829 | 882 | 8444 | 1061 |
| N73Y | 5732 | 1146 | 8745 | 1594 |

TABLE 26
Summary screening D99X & S100X substitution libraries

| | CHO Flp-In | | CHO Flp-In | |
|---|---|---|---|---|
| | huNav1.7α + β1-β2-β3 | | rhNav1.7α + β1-β2-β3 | |
| | (SEQ ID NO: 3) | | (SEQ ID NO: 4) | |
| | Mean | SD | Mean | SD |
| Substitution | MFI | MFI | MFI | MFI |
| D99A | 14187 | 4136 | 9871 | 2193 |
| D99C | 4625 | 803 | 1637 | 418 |
| D99E | 10633 | 2657 | 6153 | 1878 |
| D99F | 23749 | 3820 | 1834 | 255 |
| D99G | 23305 | 4036 | 10611 | 1456 |
| D99H | 23969 | 8888 | 11169 | 3971 |
| D99I | 4646 | 726 | 551 | 9 |
| D99K | 46514 | 619 | 1036 | 31 |
| D99L | 12797 | 251 | 742 | 103 |
| D99M | 15591 | 1834 | 1136 | 98 |
| D99N | 41774 | 971 | 45539 | 49 |
| D99P | 568 | 6 | 518 | 10 |
| D99R | 45426 | 7088 | 9917 | 2228 |
| D99S | 27582 | 442 | 32383 | 2312 |
| D99V | 12103 | 3467 | 7536 | 1541 |
| D99W | 23646 | 5065 | 8717 | 2202 |
| D99Y | 36537 | 6107 | 11155 | 2438 |
| Parental reference | 21107 | 6057 | 24705 | 5475 |
| S100A | 17866 | 3265 | 18065 | 3864 |
| S100C | 10920 | 2106 | 10646 | 3547 |
| S100D | 3674 | 572 | 2003 | 206 |
| S100E | 9252 | 711 | 6475 | 1144 |
| S100F | 30229 | 410 | 27675 | 1566 |
| S100G | 22114 | 5108 | 22287 | 6403 |
| S100K | 48169 | 1478 | 3966 | 153 |
| S100L | 18824 | 2146 | 21105 | 437 |
| S100M | 26478 | 1935 | 30669 | 2530 |
| S100Q | 33974 | 2862 | 29579 | 1025 |
| S100R | 46564 | 11444 | 12496 | 2135 |
| S100T | 23257 | 7442 | 23861 | 5720 |
| S100V | 29079 | 3063 | 30108 | 330 |
| S100W | 16328 | 1359 | 11736 | 1568 |
| S100Y | 29104 | 1100 | 18385 | 1265 |

TABLE 27
Summary screening D72X, N100cX
& T100dX substitution libraries

| | CHO Flp-In | | CHO Flp-In | |
|---|---|---|---|---|
| | huNav1.7α + β1-β2-β3 | | rhNav1.7α + β1-β2-β3 | |
| | (SEQ ID NO: 3) | | (SEQ ID NO: 4) | |
| | Mean | SD | Mean | SD |
| Substitution | MFI | MFI | MFI | MFI |
| Parental reference | 20313 | 8867 | 21158 | 8172 |
| Blanc | 516 | 21 | 462 | 19 |
| D72A | 32097 | 33 | 32753 | 1387 |
| D72C | 17787 | 10134 | 18066 | 8851 |
| D72E | 30260 | 14879 | 29532 | 13937 |
| D72F | 44931 | 24269 | 41549 | 17306 |
| D72G | 45410 | 7433 | 40171 | 4018 |
| D72I | 49815 | 15573 | 45508 | 12208 |
| D72K | 38860 | 3520 | 37695 | 3863 |
| D72L | 25424 | 11275 | 25338 | 9201 |
| D72M | 31103 | 13565 | 30170 | 11470 |
| D72P | 33230 | 11366 | 33325 | 8976 |
| D72Q | 47752 | 4735 | 43479 | 6177 |
| D72T | 38112 | 13534 | 35858 | 10233 |
| D72V | 31046 | 1671 | 30086 | 2155 |
| D72W | 44290 | 19513 | 38836 | 16236 |
| D72Y | 48641 | 19181 | 44618 | 14962 |
| N100cA | 18336 | 4353 | 16832 | 3604 |
| N100cD | 33726 | 3075 | 25954 | 3742 |
| N100cE | 21563 | 4451 | 15559 | 4511 |
| N100cF | 49642 | 6758 | 45764 | 6911 |
| N100cG | 54396 | 3864 | 46627 | 6061 |
| N100cI | 48332 | 2499 | 45805 | 2003 |
| N100cK | 46602 | 9571 | 50105 | 7223 |
| N100cL | 22679 | 3863 | 21605 | 3558 |
| N100cM | 36747 | 5344 | 35809 | 3787 |
| N100cP | 42552 | 379 | 35496 | 130 |
| N100cQ | 31049 | 966 | 28498 | 371 |
| N100cR | 39701 | 8211 | 46177 | 4971 |
| N100cS | 45457 | 167 | 43443 | 390 |
| N100cT | 38337 | 13505 | 35636 | 12150 |
| N100cV | 23442 | 3541 | 23509 | 3556 |
| N100cW | 38400 | 11435 | 37044 | 6489 |
| N100cY | 26791 | 303 | 27111 | 348 |
| T100dA | 8815 | 4912 | 5014 | 2542 |
| T100dC | 7049 | 1662 | 8567 | 2118 |
| T100dD | 3647 | 1652 | 6727 | 3269 |
| T100dE | 541 | 12 | 1562 | 384 |
| T100dF | 628 | 133 | 8704 | 7843 |
| T100dH | 19494 | 9467 | 15424 | 7021 |
| T100dI | 884 | 161 | 24959 | 17119 |
| T100dK | 519 | 39 | 462 | 31 |
| T100dL | 807 | 34 | 19047 | 1558 |
| T100dM | 7096 | 10045 | 35198 | 14877 |
| T100dP | 2934 | 840 | 20174 | 6801 |
| T100dQ | 700 | 75 | 7062 | 3009 |
| T100dR | 529 | 6 | 464 | 10 |
| T100dS | 20718 | 6040 | 23029 | 5954 |
| T100dV | 5499 | 611 | 13598 | 1603 |
| T100dW | 638 | 4 | 5362 | 810 |
| T100dY | 937 | 166 | 26415 | 9982 |

### Characterization of F0103275B05/387G04 Variants

Sequence optimization was initiated on F0103275B05 (Table 29) but later on continued on the related and improved F0103387G04 (Table 30). Likewise, affinity maturation substitutions identified for F0103275B05 were successfully transferred to F0103387G04. The variants were compared in binding FACS on human and rhesus Nav1.7α, in aSEC for possible multimerization, in OD340 for insoluble aggregate formation and in the thermal shift assay for Tm.

The thermal shift assay (TSA) was performed in a 96-well plate on the LightCycler 48011 machine (Roche). Per row, one sample was analyzed according to the following pH range: 3.5/4/4.5/5/5.5/6/6.5/7/7.5/8/8.5/9. Per well, 5 μl of sample (0.8 mg/ml in PBS) was added to 5 μL of Sypro Orange (40× in MilliQ water; Invitrogen cat. No. 56551) and 10 μL of buffer (100 mM phosphate, 100 mM borate, 100 mM citrate and 115 mM NaCl with a pH ranging 3.5 to 9). The applied temperature gradient (37 to 99° C. at a rate of 0.03° C./s) induces unfolding of the ISVDs whereby their hydrophobic patches become exposed. Sypro Orange binds to those hydrophobic patches, resulting in an increase in fluorescence intensity (Ex/Em=465/580 nm). The inflection point of the first derivative of the fluorescence intensity curve at pH 7 serves as a measure of the melting temperature (Tm).

Table 28 summarizes the effects of the explored substitutions.

TABLE 28
Overview of F0103387G04 substitutions

| | huNav1.7α | rhNav1.7α | | | | |
|---|---|---|---|---|---|---|
| | EC50 | EC50 | Tm | aSEC | | |
| | fold change | fold change | difference | behavior | OD340 | |
| | compared to | compared to | compared to | compared to | compared to | Retain |
| Substitution | reference | reference | reference | reference | reference | substitution |
| L11V | = | = | −2 | = | = | Y |
| (L11V, T83R, V89L) | | | | | | |
| A12V | ND | ND | ND | ND | ND | Y |
| K33R | = | + | −4 | = | = | Y |
| (K33R, S50Y, S56D, | | | | | | |
| N93R) | | | | | | |
| R39Q | − | − | +3 | = | = | Y |
| S50Y | = | + | −4 | = | = | Y |
| (K33R, S50Y, S56D, | | | | | | |
| N93R) | | | | | | |
| S56D | = | + | −4 | = | = | Y |
| (K33R, S50Y, S56D, | | | | | | |
| N93R) | | | | | | |
| T60A | = | = | +3 | = | = | Y |
| D72G | + | + | ND | ND | ND | Y |
| D72Q | + | + | ND | ND | ND | N |
| G73N | −2 | −2 | −2 | = | = | N |
| G73A | − | − | −3 | = | = | N |
| G73R | + | + | −4 | = | = | N |
| R76N | −2 | −2 | −2 | = | = | N |
| R76_V78insT | −3 | −6 | +5 in | = | = | N |
| | | | 275B05 | | | |
| | | | −5 in | | | |
| | | | 387G04 | | | |
| W78V | = | = | −6 | = | = | Y |
| S79Y | = | = | = | = | = | Y |
| T83R | = | = | −2 | = | | Y |
| (L11V, T83R, V89L) | | | | | | |
| V89L | = | = | −2 | = | = | Y |
| (L11V, T83R, V89L) | | | | | | |
| N93R | = | ++ | −4 | = | = | Y |
| (K33R, S50Y, S56D, | | | | | | |
| N93R) | | | | | | |
| D99S | +2 | +2 | −1 | = | = | Y |
| D99R | + | + | proteolytic degradation | | | N |
| D99N | +2 | +2 | −2 | = | = | N |
| S100R | + | + | proteolytic degradation | | | N |
| S100V | −2 | −2 | +1 | = | = | N |
| N100cG | + | + | = | = | = | Y |
| N100cI | − | − | −1 | = | = | N |

ND, not determined

TABLE 29
Characterization of F0103275B05 variants
Part 1

| | | | | | | | | | | | CHO | |
|---|---|---|---|---|---|---|---|---|---|---|---|---|
| | | | | | | | | | | | FlpIn | CHO |
| | | | | | | | | | | | huNav1.7α- | FlpIn |
| | | | | | | | | | | | β1-β2- | rhNav1.7α- |
| | | | | | | | | | | | (SEQ ID | β1-β2-β3 |
| | | | | | | | | | | | NO: 3) | (SEQ ID |
| | | | | | | | | | | | β3 | NO: 4) |
| ID # | L11 | S33R | R39 | S50Y | S56D | R76 | 77 | T83 | V89 | N93R | EC50 [M] | EC50 [M] |
| F010301461 | . | R | . | Y | D | . | — | . | . | R | ND | ND |
| F010301635 | V | . | Q | . | . | . | — | R | L | . | ND | ND |
| F010301636 | V | . | . | . | . | N | — | R | L | . | ND | ND |
| F010301637 | V | . | . | . | . | . | — | R | L | . | 5.6E−08 | ND |
| F010301638 | V | . | Q | . | . | N | — | R | L | . | ND | ND |
| F010301639 | V | . | Q | . | . | . | T | R | L | . | ND | ND |
| F010301640 | V | . | . | . | . | . | T | R | L | . | ND | ND |
| F010301641 | V | . | . | . | . | N | T | R | L | . | ND | ND |
| F010301642 | V | . | Q | . | . | N | T | R | L | . | ND | ND |
| F010301652 | V | R | . | Y | D | N | — | R | L | R | 6.1E−08 | 2.4E−08 |
| F010301653 | V | R | . | Y | D | . | — | R | L | R | 3.6E−08 | 1.6E−08 |
| F010301654 | V | R | Q | Y | D | . | — | R | L | R | 3.2E−08 | 1.8E−08 |
| F010301655 | V | R | Q | Y | D | N | — | R | L | R | 4.6E−08 | 2.6E−08 |
| F0103275B05 | . | . | . | . | . | . | — | . | . | . | 6.6E−08 | — |

Part 2

| | | | HEKa/b 1 | | |
|---|---|---|---|---|---|
| | HEK FlpIn | HEK | (SEQ ID | | |
| | Nav157ch14- | FlpIn | NO: 40) | HEKa | |
| | β1-β2-β3 | Nav157ch14 | huNav1.7α | huNav1.7α | |
| | (SEQ ID | (SEQ ID | (SEQ ID | (SEQ ID | |
| | NO: 20) | NO: 20) | NO: 1) | NO: 1) | Tm |
| ID # | EC50 [M] | EC50 [M] | EC50 [M] | EC50 [M] | [° C.] |
| F010301461 | ND | ND | ND | ND | 64 |
| F010301635 | 3.5E−08 | 1.6E−08 | 3.1E−08 | 1.9E−08 | 68 |
| F010301636 | 4.5E−08 | 1.4E−08 | 3.0E−08 | 1.7E−08 | 64 |
| F010301637 | 2.9E−08 | 1.1E−08 | 3.0E−08 | 1.3E−08 | 66 |
| F010301638 | 5.0E−08 | 1.8E−08 | 3.2E−08 | 2.3E−08 | 67 |
| F010301639 | — | — | 9.4E−08 | — | 72 |
| F010301640 | — | — | 6.5E−08 | — | 69 |
| F010301641 | 3.5E−07 | 3.1E−07 | 2.3E−07 | 3.8E−07 | 70 |
| F010301642 | — | — | — | — | 73 |
| F010301652 | ND | ND | 3.8E−08 | 2.4E−08 | 60 |
| F010301653 | ND | ND | 2.9E−08 | 1.5E−08 | 62 |
| F010301654 | ND | ND | 2.5E−08 | 1.8E−08 | 65 |
| F010301655 | ND | ND | 2.8E−08 | 1.9E−08 | 63 |
| F0103275B05 | 3.3E−08 | 1.2E−08 | 3.9E−08 | 1.4E−08 | 68 |

ND, not determined

TABLE 30
Characterization of F0103387G04 variants

Part 1

| ID # | L11 | A12 | K33 | R39 | S50 | S56 | T60 | D72 | G73 | R76 |
|---|---|---|---|---|---|---|---|---|---|---|
| F010301656 | . | . | R | . | Y | D | . | . | . | . |
| F010301840 | V | V | R | Q | Y | D | . | . | . | . |
| F010301841 | V | V | R | Q | Y | D | . | . | . | . |
| F010301842 | V | V | R | Q | Y | D | A | . | . | . |
| F010301843 | V | V | R | Q | Y | D | . | . | N | . |
| F010301844 | V | V | R | Q | Y | D | . | . | . | N |
| F010301845 | V | V | R | Q | Y | D | . | . | . | . |
| F010301846 | V | V | R | Q | Y | D | . | . | . | . |
| F010301847 | V | V | R | Q | Y | D | A | . | N | N |
| F010301848 | V | V | R | Q | Y | D | A | . | N | . |
| F010301865 | V | V | R | Q | Y | D | A | . | . | . |
| F010301866 | V | V | R | Q | Y | D | A | . | . | N |
| F010302310 | V | V | R | Q | Y | D | A | | A | . |
| F010302311 | V | V | R | Q | Y | D | A | | R | . |
| F010302312 | V | V | R | Q | Y | D | A | . | . | . |
| F010302313 | V | V | R | Q | Y | D | A | . | A | . |
| F010302314 | V | V | R | Q | Y | D | A | . | R | . |
| F010302315 | V | V | R | Q | Y | D | A | | . | . |
| F010302316 | V | V | R | Q | Y | D | A | | A | . |
| F010302317 | V | V | R | Q | Y | D | A | | R | . |
| F010302318 | V | V | R | Q | Y | D | A | | . | . |
| F010302319 | V | V | R | Q | Y | D | A | | A | . |
| F010302320 | V | V | R | Q | Y | D | A | . | A | . |
| F010302321 | V | V | R | Q | Y | D | A | | R | . |
| F010302322 | V | V | R | Q | Y | D | A | | . | . |
| F010302323 | V | V | R | Q | Y | D | A | | A | . |
| F010302324 | V | V | R | Q | Y | D | A | | R | . |
| F010302325 | V | V | R | Q | Y | D | A | . | R | . |
| F010302326 | V | V | R | Q | Y | D | A | | . | . |
| F010302327 | V | V | R | Q | Y | D | A | | A | . |
| F010302328 | V | V | R | Q | Y | D | A | | R | . |
| F010302329 | V | V | R | Q | Y | D | A | | . | . |
| F010302330 | V | V | R | Q | Y | D | A | | A | . |
| F010302331 | V | V | R | Q | Y | D | A | | R | . |
| F010302332 | V | V | R | Q | Y | D | A | | . | . |
| F010302370 | V | V | R | Q | Y | D | A | . | R | . |
| F010302371 | V | V | R | Q | Y | D | A | . | R | . |
| F010302372 | V | V | R | Q | Y | D | A | . | R | . |
| F010302383 | V | V | R | Q | Y | D | A | G | . | . |
| F010302384 | V | V | R | Q | Y | D | A | G | . | . |
| F010302385 | V | V | R | Q | Y | D | A | Q | . | . |
| F010302386 | V | V | R | Q | Y | D | A | Q | . | . |
| F0103387G04 | . | . | . | . | . | . | . | . | . | . |

Part 1

| ID # | 77 | W78 | S79 | T83 | V89 | N93 | D99 | S100 | N100c |
|---|---|---|---|---|---|---|---|---|---|
| F010301656 | — | . | . | . | . | R | . | . | . |
| F010301840 | T | . | . | R | L | R | . | . | . |
| F010301841 | — | . | . | R | L | R | . | . | . |
| F010301842 | — | . | . | R | L | R | . | . | . |
| F010301843 | — | . | . | R | L | R | . | . | . |
| F010301844 | — | . | . | R | L | R | . | . | . |
| F010301845 | — | V | . | R | L | R | . | . | . |
| F010301846 | — | . | Y | R | L | R | . | . | . |
| F010301847 | — | V | Y | R | L | R | . | . | . |
| F010301848 | — | V | Y | R | L | R | . | . | . |
| F010301865 | — | V | Y | R | L | R | . | . | . |
| F010301866 | — | V | Y | R | L | R | . | . | . |
| F010302310 | — | V | Y | R | L | R | R | R | . |
| F010302311 | — | V | Y | R | L | R | R | R | . |
| F010302312 | — | V | Y | R | L | R | . | . | I |
| F010302313 | — | V | Y | R | L | R | . | . | I |
| F010302314 | — | V | Y | R | L | R | . | . | I |
| F010302315 | — | V | Y | R | L | R | R | . | I |
| F010302316 | — | V | Y | R | L | R | R | . | I |
| F010302317 | — | V | Y | R | L | R | R | . | I |
| F010302318 | — | V | Y | R | L | R | . | R | I |
| F010302319 | — | V | Y | R | L | R | . | R | I |
| F010302320 | — | V | Y | R | L | R | . | . | . |
| F010302321 | — | V | Y | R | L | R | . | R | I |
| F010302322 | — | V | Y | R | L | R | R | R | I |
| F010302323 | — | V | Y | R | L | R | R | R | I |
| F010302324 | — | V | Y | R | L | R | R | R | I |
| F010302325 | — | V | Y | R | L | R | . | . | . |
| F010302326 | — | V | Y | R | L | R | R | . | . |
| F010302327 | — | V | Y | R | L | R | R | . | . |
| F010302328 | — | V | Y | R | L | R | R | . | . |
| F010302329 | — | V | Y | R | L | R | . | R | . |
| F010302330 | — | V | Y | R | L | R | . | R | . |
| F010302331 | — | V | Y | R | L | R | . | R | . |
| F010302332 | — | V | Y | R | L | R | R | R | . |
| F010302370 | — | V | Y | R | L | R | S | . | I |
| F010302371 | — | V | Y | R | L | R | N | V | I |
| F010302372 | — | V | Y | R | L | R | . | V | I |
| F010302383 | — | V | Y | R | L | R | S | . | G |
| F010302384 | — | V | Y | R | L | R | S | . | I |
| F010302385 | — | V | Y | R | L | R | S | . | G |
| F010302386 | — | V | Y | R | L | R | S | . | I |
| F0103387G04 | — | . | . | . | . | . | . | . | . |

Part 2

| | CHO | | | | |
|---|---|---|---|---|---|
| | FlpIn | CHO | CHO | CHO | |
| | huNav1.7α + | FlpIn | FlpIn | FlpIn | |
| | β1-β2- | huNav1.7α + | rhNav1.7α- | rhNav1.7α- | |
| | β3 (SEQ | β1-β2- | β1 + β2- | β1 + β2- | |
| | ID NO: | β3(SEQ | β3 (SEQ | β3 (SEQ | |
| | 3) EC50 | ID NO: 3) | ID NO: 4) | ID NO: | |
| ID # | [M] | Bmax | EC50 [M] | 4) Bmax | Tm [° C.] |
| F010301656 | 1.7E−08 | 100% | 8.0E−09 | 100% | 72 |
| F010301840 | 4.7E−08 | 100% | 4.8E−08 | 50% | 70 |
| F010301841 | 1.4E−08 | 100% | 7.6E−09 | 100% | 75 |
| F010301842 | 1.5E−08 | 100% | 7.9E−09 | 100% | 78 |
| F010301843 | 1.8E−08 | 100% | 9.3E−09 | 100% | 74 |
| F010301844 | 2.3E−08 | 100% | 1.1E−08 | 100% | 73 |
| F010301845 | 1.7E−08 | 100% | 8.0E−09 | 100% | 69 |
| F010301846 | 2.1E−08 | 100% | 1.1E−08 | 100% | 75 |
| F010301847 | 6.7E−08 | 100% | 2.9E−08 | 100% | 70 |
| F010301848 | 4.4E−08 | 100% | 1.9E−08 | 100% | 73 |
| F010301865 | 2.0E−08 | 100% | 1.1E−08 | 100% | 75 |
| F010301866 | 3.8E−08 | 100% | 1.8E−08 | 100% | 73 |
| F010302310 | ND | ND | ND | ND | ND |
| F010302311 | ND | ND | ND | ND | ND |
| F010302312 | 2.4E−08 | 100% | 1.8E−08 | 100% | 74 |
| F010302313 | 3.8E−08 | 100% | 3.4E−08 | 96% | 71 |
| F010302314 | 1.6E−08 | 100% | 1.4E−08 | 98% | 70 |
| F010302315 | 7.2E−09 | 100% | 3.9E−08 | 23% | ND |
| F010302316 | 8.2E−09 | 91% | — | — | ND |
| F010302317 | 6.8E−09 | 91% | 1.9E−08 | 13% | ND |
| F010302318 | 1.1E−08 | 89% | — | — | ND |
| F010302319 | 1.5E−08 | 88% | — | — | ND |
| F010302320 | 3.0E−08 | 99% | 2.1E−08 | 100% | 71 |
| F010302321 | 7.9E−09 | 91% | — | — | ND |
| F010302322 | ND | ND | ND | ND | ND |
| F010302323 | ND | ND | ND | ND | ND |
| F010302324 | 2.9E−08 | 42% | — | — | ND |
| F010302325 | 1.3E−08 | 96% | 8.7E−09 | 99% | 71 |
| F010302326 | 6.2E−09 | 89% | 1.8E−08 | 32% | ND |
| F010302327 | 7.0E−09 | 87% | 9.3E−08 | 7% | ND |
| F010302328 | ND | ND | ND | ND | ND |
| F010302329 | 7.6E−09 | 94% | 1.9E−08 | 44% | ND |
| F010302330 | 1.0E−08 | 93% | 2.0E−07 | 23% | ND |
| F010302331 | 6.0E−09 | 90% | 2.4E−08 | 38% | ND |
| F010302332 | ND | ND | ND | ND | ND |
| F010302370 | 9.0E−09 | 100% | 7.3E−09 | 100% | 69 |
| F010302371 | 9.4E−09 | 100% | 1.0E−08 | 100% | 68 |
| F010302372 | 1.7E−08 | 100% | 2.0E−08 | 100% | 70 |
| F010302383 | 6.8E−09 | 100% | 4.5E−09 | 100% | 73 |
| F010302384 | 6.2E−09 | 100% | 4.3E−09 | 100% | 72 |
| F010302385 | 7.9E−09 | 100% | 5.0E−09 | 100% | 74 |
| F010302386 | 7.3E−09 | 100% | 4.9E−09 | 100% | 72 |
| F0103387G04 | 1.7E−08 | 100% | 8.7E−08 | 10% | 76 |

ND, not determined

### Selection of an F0103387G04 Sequence Optimization Variant

Variant F010302383 was selected as the final sequence optimization variant of F0103387G04 (see F0103387G04 SO in FIG. 28). It boasts a 2- and 20-fold improved binding on huNav1.7α and rhNav1.7α respectively, as well as comparable aSEC and OD340 nm behavior and a slightly reduced thermal stability (Table 31). In vitro electrophysiology experiments confirmed the low nM potency on huNav1.7α and rhNav1.7α and selectivity over Nav1.4, Nav1.5α and Nav1.6α. All PTM liabilities (Table 24) were successfully substituted. Cell-free fermentation expression titers at CMC of the corresponding monovalent tagless variant F01032396 in P. pastoris were 3.6 g/L.

TABLE 31
Sequence optimization variant of F0103387G04
Part 1

| | CHO | CHO | CHO | | | | | |
|---|---|---|---|---|---|---|---|---|
| | FlpIn | FlpIn | FlpIn | HEK | HEK | | | |
| | huNav1.7α + | rhNav1.7α + | rhNav1.7α + | huNav1.7α + | rhNav1.7α + | HEK | HEK | HEK |
| | β1-β2-β3 | β1-β2-β3 | β1-β2-β3 | β1 | β1-β2-β3 | huNav1.4α | huNav1.5α | huNav1.6α |
| | (SEQ ID | (SEQ ID | (SEQ ID | (SEQ ID | (SEQ ID | (SEQ ID | (SEQ ID | (SEQ ID |
| | NO: 3) | NO: 4) | NO: 4) | NO: 44) | NO: 4) | NO: 1) | NO: 27) | NO: 28) |
| ID # | EC50 [M] | EC50 [M] | Bmax | IC50 [M] | IC50 [M] | IC50 [M] | IC50 [M] | IC50 [M] |
| F0103387G04 | 1.7E−08 | 8.7E−08 | 10% | ND | ND | ND | ND | ND |
| F010302383 | 6.8E−09 | 4.5E−09 | 100% | 2.2E−08 | 3.0E−09 | — | — | — |

—, no activity observed @ 7 μM;
ND, not determined

Part 2

| ID # | Tm [° C.] | Tagg [° C.] | aSEC | OD340 nm | AbM | Kabat |
|---|---|---|---|---|---|---|
| F0103387G04 | 76 | ND | ok | ok | 81% | 78% |
| F010302383 | 73 | 69 | ok | ok | 83% | 79% |

ND, not determined

### Example 9

### Sequence Optimization of F0103387G05

Several PTM substitution libraries were generated based on the accelerated stress data summarized in Table 32 and screened as crude periplasmic extracts in binding FACS on human Nav1.7α.

TABLE 32
Results accelerated stress experiments performed on F0103387G05 variants

| | | | | Modification |
|---|---|---|---|---|
| ID # | Description | Site | Stress condition | observed |
| F0103387G05 | F0103387G05- | NA | 1 week @ 45° | 0.3% increase |
| | FLAG3-HIS6 | | C., ±1 mg/mL | of pre-peak |
| | | | in D-PBS | (SE-HPLC) |
| F010301456 | F0103387G05-HIS6 | CD | 4 weeks @ −20, | 0.8-6.1% |
| | | R2 | 25 and 40° C. | isomerization |
| F010301456 | F0103387G05-HIS6 | N73 | 4 weeks @ −20, | 1.2-13% |
| | | | 25 and 40° C. | |
| F010301949 | F0103387G05(L11V, | CD | 4 weeks @ −20, | 1-9% |
| | A14P, D23A, H37Y, | R2 | 25 and 40° C. | isomerization |
| | G40A, A41P, D58G, | | | |
| | N82bS, N83R, V89L, | | | |
| | R105Q)-HIS6 | | | |
| F010301949 | F0103387G05(L11V, | N73 | 4 weeks @ −20, | 0.7-9% |
| | A14P, D23A, H37Y, | | 25 and 40° C. | |
| | G40A, A41P, D58G, | | | |
| | N82bS, N83R, | | | |
| | V89L, R105Q)- | | | |
| | HIS6 | | | |
| F010302391 | F0103387G05(L11V, | NA | 1 week @ 45° | 0.2% increase |
| | A14P, D23A, H37Y, | | C., ±1 mg/mL | of pre-peak |
| | G40A, A41P, D53G, | | in D-PBS | (SE-HPLC) |
| | D54G, D58G, N82bS, | | | |
| | N83R, V89L, R105Q)- | | | |
| | FLAG3-HIS6 | | | |

ND, not determined

N73Q substitution resulted in a better binding profile compared to the parental reference (Table 33) and was further evaluated, as well as N73A and N73Y substitutions.
Because of the available choices to substitute N73, no mutations for A74 were further evaluated.

TABLE 33
Summary screening of N73X & A74 substitution libraries

CHO Flp-In
huNav1.7α + β1-β2-β3
(SEQ ID NO: 3)

| | Mean | SD |
|---|---|---|
| Substitution | MFI | MFI |
| A74D | 72131 | 8952 |
| A74E | 67339 | 6350 |
| A74I | 76360 | 7842 |
| A74K | 99046 | 1123 |
| A74L | 70981 | 6848 |
| A74N | 80896 | 11713 |
| A74P | 63748 | 2925 |
| A74Q | 75053 | 9885 |
| A74S | 57716 | 7270 |
| A74T | 66295 | 10425 |
| A74W | 40642 | 8566 |
| Blanc | 570 | NA |
| Negative control | 566 | 12 |
| Parental reference | 48046 | 8443 |
| N73A | 84208 | 12907 |
| N73D | 77600 | 8148 |
| N73E | 64666 | 7724 |
| N73F | 60283 | 14931 |
| N73G | 84896 | 8452 |
| N73H | 68524 | 2512 |
| N73I | 81380 | 16558 |
| N73K | 85809 | 21302 |
| N73L | 70525 | 14051 |
| N73M | 87197 | 8041 |
| N73P | 58705 | 45951 |
| N73Q | 79497 | 12894 |
| N73R | 66654 | 8484 |
| N73S | 85111 | 2933 |
| N73T | 71638 | 13462 |
| N73V | 84743 | 7727 |
| N73Y | 86218 | 8813 |

HEK293 human Nav1.7α
(SEQ ID NO: 1)

| | Mean | SD |
|---|---|---|
| Substitution | MFI | MFI |
| A74D | 33271 | 2071 |
| A74E | 28963 | 2627 |
| A74I | 33074 | 3560 |
| A74K | 38376 | 722 |
| A74L | 27756 | 4959 |
| A74N | 34952 | 2025 |
| A74P | 28080 | 948 |
| A74Q | 31394 | 3986 |
| A74S | 29160 | 2997 |
| A74T | 30375 | 4492 |
| A74W | 20460 | 4869 |
| Blanc | 734 | NA |
| Negative control | 723 | 2 |
| Parental reference | 22340 | 3535 |
| N73A | 35560 | 5590 |
| N73D | 32744 | 2730 |
| N73E | 29576 | 3544 |
| N73F | 27251 | 5503 |
| N73G | 35331 | 3769 |
| N73H | 31210 | 1722 |
| N73I | 36497 | 5563 |
| N73K | 32267 | 6201 |
| N73L | 34819 | 2876 |
| N73M | 38463 | 2348 |
| N73P | 25112 | 18794 |
| N73Q | 33609 | 3748 |
| N73R | 30872 | 4401 |
| N73S | 36285 | 1224 |
| N73T | 32687 | 4647 |
| N73V | 37400 | 1183 |
| N73Y | 36734 | 5290 |

HEK293 human
Nav1.7α + β1
(SEQ ID NO: 44)

| | Mean | SD |
|---|---|---|
| Substitution | MFI | MFI |
| A74D | 79560 | 6225 |
| A74E | 74537 | 1165 |
| A74I | 76639 | 1998 |
| A74K | 84166 | 964 |
| A74L | 75554 | 2070 |
| A74N | 81639 | 5349 |
| A74P | 66190 | 2235 |
| A74Q | 78895 | 5333 |
| A74S | 65093 | 7925 |
| A74T | 72355 | 5048 |
| A74W | 49009 | 11489 |
| Blanc | 789 | NA |
| Negative control | 778 | 6 |
| Parental reference | 59514 | 8475 |
| N73A | 83244 | 10774 |
| N73D | 82260 | 3026 |
| N73E | 76352 | 3918 |
| N73F | 64305 | 10544 |
| N73G | 79190 | 3307 |
| N73H | 70459 | 3060 |
| N73I | 79431 | 7248 |
| N73K | 79446 | 16278 |
| N73L | 75337 | 11011 |
| N73M | 84223 | 4072 |
| N73P | 60089 | 45450 |
| N73Q | 78260 | 5434 |
| N73R | 67882 | 4153 |
| N73S | 79692 | 2217 |
| N73T | 76277 | 8140 |
| N73V | 84341 | 3090 |
| N73Y | 85910 | 1947 |

NA, not applicable

### Characterization of F0103387G05 Variants

Affinity maturation substitutions that improved the binding of F0103387G05 were transferred to the sequence optimized variants. These variants were compared (Table 35) in binding FACS on human Nav1.7α, in aSEC for possible multimerization, in OD340 for insoluble aggregate formation and in the thermal shift assay for Tm. Table 34 summarizes the effects of the explored substitutions.

TABLE 34
Overview of F0103387G05 substitutions
Part 1

| | huNav1.7α + β1 | huNav1.7α | huNav1.7α |
|---|---|---|---|
| | (SEQ ID NO: 44) | (SEQ ID NO: 1) | (SEQ ID NO: 1) |
| | EC50 | without EC50 | without Bmax |
| | fold change | fold change | fold change |
| | compared to | compared to | compared to |
| Substitution | reference | reference | reference |
| L11V(L11V, A14P, N82bS, | = | = | = |
| N83R, V89L, R105Q) | | | |
| A14P(L11V, A14P, N82bS, | = | = | = |
| N83R, V89L, R105Q) | | | |
| D23A | + | + | = |
| H37Y | = | = | = |
| G40A | = | = | = |
| A41P | = | = | = |
| F47L | = | −2 | − |
| D53G (D53G, D54G) | + | + | = |
| D54G (D53G, D54G) | + | + | = |
| D58G | + | + | = |
| N73A | = | = | = |
| N73Q | = | = | = |
| N73Y | = | = | = |
| N82bS(L11V, A14P, N82bS, | = | = | = |
| N83R, V89L, R105Q) | | | |
| N83R(L11V, A14P, N82bS, | = | = | = |
| N83R, V89L, R105Q) | | | |
| V89L(L11V, A14P, N82bS, | = | = | = |
| N83R, V89L, R105Q) | | | |
| E93N | = | −20 | −4 |
| R105Q(L11V, A14P, N82bS, | = | = | = |
| N83R, V89L, R105Q) | | | |

Part 2

| | Tm | aSEC | | |
|---|---|---|---|---|
| | difference | behavior | OD340 | |
| | compared to | compared to | compared to | Retain |
| Substitution | reference | reference | reference | substitution |
| L11V(L11V, A14P, N82bS, | −2 | = | = | Y |
| N83R, V89L, R105Q) | | | | |
| A14P(L11V, A14P, N82bS, | −2 | = | = | Y |
| N83R, V89L, R105Q) | | | | |
| D23A | +4 | = | = | Y |
| H37Y | = | = | = | Y |
| | ++ at low | | | |
| | pH (FIG. 3) | | | |
| G40A | +1 | = | = | Y |
| A41P | +1 | = | = | Y |
| F47L | −4 | = | = | N |
| D53G (D53G, D54G) | −4 | ND | ND | Y |
| D54G (D53G, D54G) | −4 | ND | ND | Y |
| D58G | −4 | = | = | Y |
| N73A | = | = | = | N |
| N73Q | = | = | = | N |
| N73Y | −1 | = | = | N |
| N82bS(L11V, A14P, N82bS, | −2 | = | = | Y |
| N83R, V89L, R105Q) | | | | |
| N83R(L11V, A14P, N82bS, | −2 | = | = | Y |
| N83R, V89L, R105Q) | | | | |
| V89L(L11V, A14P, N82bS, | −2 | = | = | Y |
| N83R, V89L, R105Q) | | | | |
| E93N | −6 | = | = | N |
| R105Q(L11V, A14P, N82bS, | −2 | = | = | Y |
| N83R, V89L, R105Q) | | | | |

ND, not determined

TABLE 35
Characterization of F010387G05 variants
Part 1

| ID # | L11 | A14 | D23 | H37 | G40 | A41 | F47 | D53 | D54 | D58 | N73 | N82b | N83 | V89 | E93 | R105 |
|---|---|---|---|---|---|---|---|---|---|---|---|---|---|---|---|---|
| F010301556 | . | . | A | . | . | . | . | G | G | G | . | . | . | . | . | . |
| F010301563 | . | . | A | . | . | . | . | . | . | G | . | . | . | . | . | . |
| F010301643 | Q | P | A | . | . | . | . | . | . | . | . | S | R | L | . | Q |
| F010301644 | V | P | . | Y | . | . | . | . | . | . | . | S | R | L | . | Q |
| F010301645 | V | P | . | . | A | . | . | . | . | . | . | S | R | L | . | Q |
| F010301646 | V | P | . | . | . | P | . | . | . | . | . | S | R | L | . | Q |
| F010301647 | V | P | . | . | . | . | L | . | . | . | . | S | R | L | . | Q |
| F010301648 | V | P | . | . | . | . | . | . | . | . | . | S | R | L | N | Q |
| F010301649 | V | P | . | . | . | . | . | . | . | . | . | S | R | L | . | Q |
| F010301849 | V | P | A | . | A | P | . | . | . | G | . | S | R | L | . | Q |
| F010301850 | V | P | A | Y | A | P | . | . | . | G | . | S | R | L | . | Q |
| F010302307 | V | P | A | Y | A | P | . | . | . | G | A | S | R | L | . | Q |
| F010302308 | V | P | A | Y | A | P | . | . | . | G | Y | S | R | L | . | Q |
| F010302309 | V | P | A | Y | A | P | . | . | . | G | Q | S | R | L | . | Q |
| F010302391 | V | P | A | Y | A | P | . | G | G | G | . | S | R | L | . | Q |
| F010302392 | V | P | A | Y | A | P | . | G | G | G | Q | S | R | L | . | Q |
| F0103387G05 | . | . | . | . | . | . | . | . | . | . | . | . | . | . | . | . |

Part 2

| | HEK | | | HEKa/β1 | | | HEK |
|---|---|---|---|---|---|---|---|
| | FlpIn | HEK | | (SEQ ID | | | FlpIn |
| | Nav157ch14- | FlpIn | HEK | NO: 40) | HEKa | | Nav1.7α + |
| | β1-β2- | Nav157ch14 | FlpIn | Nav1.7α | Nav1.7α | HEKa | β1- β2- |
| | β3 (SEQ | (SEQ ID | Nav157ch14 | (SEQ ID | (SEQ ID | Nav1.7α | β3 (SEQ |
| | ID NO: | NO: 20) | (SEQ ID | NO: 1) | NO: 1) | (SEQ ID | ID NO: 3) |
| | 20) EC50 | EC50 | NO: 19) | EC50 | EC50 | NO: 1) | EC50 |
| ID # | [M] | [M] | Bmax | [M] | [M] | Bmax | [M] |
| F010301556 | ND | ND | ND | 1.4E−09 | 1.8E−09 | ND | ND |
| F010301563 | ND | ND | ND | 1.0E−09 | 1.5E−09 | ND | ND |
| F010301643 | 3.6E−09 | 3.7E−09 | 100% | 3.4E−09 | 4.1E−09 | 100% | ND |
| F010301644 | 4.9E−09 | 4.4E−09 | 100% | 5.0E−09 | 5.7E−09 | 100% | ND |
| F010301645 | 4.6E−09 | 3.9E−09 | 100% | 4.2E−09 | 4.7E−09 | 100% | ND |
| F010301646 | 4.2E−09 | 3.1E−09 | 100% | 4.1E−09 | 4.4E−09 | 100% | ND |
| F010301647 | 4.7E−09 | 1.2E−08 | 80% | 4.0E−09 | 8.0E−09 | 75% | ND |
| F010301648 | 3.4E−09 | 2.1E−07 | 30% | 3.2E−09 | 8.9E−08 | 25% | ND |
| F010301649 | 4.2E−09 | 3.4E−09 | 100% | 4.1E−09 | 5.3E−09 | 100% | ND |
| F010301849 | ND | ND | ND | 1.8E−09 | 3.0E−09 | 100% | ND |
| F010301850 | 3.0E−09 | 5.4E−09 | ND | 1.8E−09 | 2.8E−09 | 100% | 1.9E−09 |
| F010302307 | ND | ND | ND | 1.9E−09 | 2.8E−09 | 100% | 2.9E−09 |
| F010302308 | ND | ND | ND | 1.3E−09 | 1.8E−09 | 100% | 2.0E−09 |
| F010302309 | ND | ND | ND | 1.5E−09 | 2.2E−09 | 100% | 2.3E−09 |
| F010302391 | 2.3E−09 | 2.7E−09 | 100% | 2.0E−09 | 1.9E−09 | 100% | ND |
| F010302392 | ND | ND | ND | ND | ND | ND | ND |
| F0103387G05 | 3.6E−09 | 2.8E−09 | 100% | 1.9E−09 | 3.1E−09 | 100% | 2.7E−09 |

ND, not determined

Part 3

| ID # | Tm [° C.] | aSEC | OD340 nm |
|---|---|---|---|
| F010301556 | 69 | ND | ND |
| F010301563 | 74 | ND | ND |
| F010301643 | 76 | ok | ok |
| F010301644 | 71 | ok | ok |
| F010301645 | 73 | ok | ok |
| F010301646 | 73 | ok | ok |
| F010301647 | 68 | ok | ok |
| F010301648 | 66 | ok | ok |
| F010301649 | 72 | ok | ok |
| F010301849 | 76 | ok | ok |
| F010301850 | 76 | ok | ok |
| F010302307 | 76 | ok | ok |
| F010302308 | 75 | ok | ok |
| F010302309 | 76 | ok | ok |
| F010302391 | 73 | ok | ok |
| F010302392 | ND | ND | ND |
| F0103387G05 | 74 | ok | ok |

ND, not determined
ok, OD360 is acceptable

### Selection of an F0103387G05 Sequence Optimization Variant

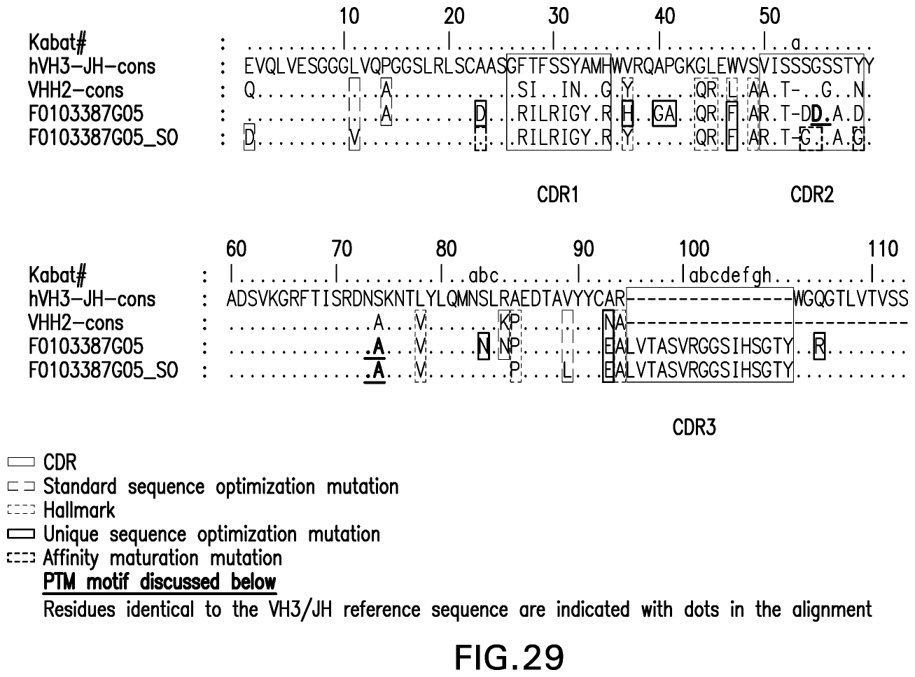

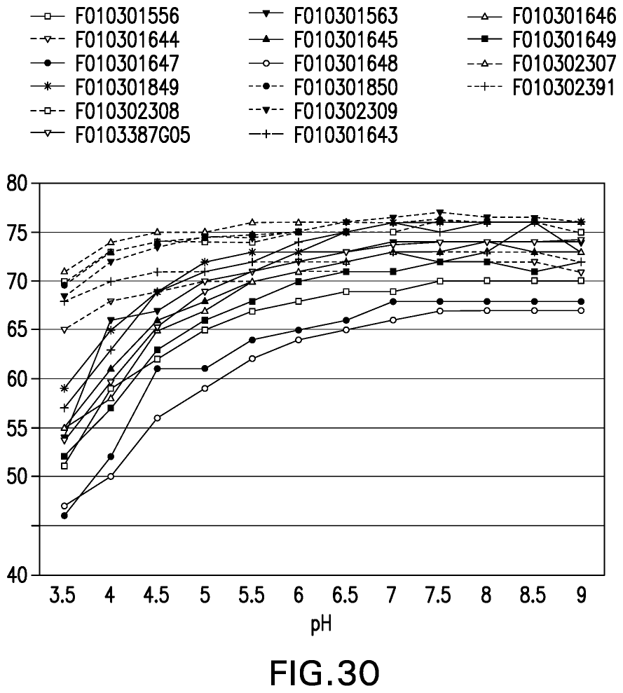

Variant F010302391 was selected as the final sequence optimization variant of F0103387G05 (see F0103387G05 SO in FIG. 29). It boasts a comparable binding on human Nav1.7α, as well as comparable aSEC and OD340 nm behavior, an improved thermal stability at low pH and a reduced Tagg (Table 36 and FIG. 30). In vitro electrophysiology experiments confirmed the low nM potency on huNav1.7α and selectivity over Nav1.4α, Nav1.5α, and Nav1.6α. All PTM liabilities (Table 32) were substituted with the exception of N73. Cell-free fermentation expression titers at CMC of the corresponding monovalent tagless variant F010302400 in P. pastoris were 2.5 g/L.

TABLE 36
Sequence optimization variant of F010387G05
Part 1

| | HEK FlpIn | | HEKa/β1 | |
|---|---|---|---|---|
| | Nav157ch14- | HEK FlpIn | (SEQ ID NO: | |
| | β1-β2- β3 | Nav157ch14 | 40) Nav1.7α | HEKa Nav1.7α |
| | (SEQ ID NO: | (SEQ ID NO: | (SEQ ID NO: | (SEQ ID NO: 1) |
| ID # | 20) EC50 [M] | 19) EC50 [M] | 1) EC50 [M] | EC50 [M] |
| F0103387G05 | 3.6E−09 | 2.8E−09 | 1.9E−09 | 3.1E−09 |
| F010302391 | 2.3E−09 | 2.7E−09 | 2.0E−09 | 1.9E−09 |

—, no activity observed @ 7 μM

Part 2

| | | HEK | HEK | HEK | HEK |
|---|---|---|---|---|---|
| | HEK | rhNav1.7α + | huNav1.4α | huNav1.5α | huNav1.6α |
| | huNav1.7α + β1 | β1-β2-β3 | (SEQ ID | (SEQ ID | (SEQ ID |
| | (SEQ ID NO: | (SEQ ID | NO: 26) | NO: 27) | NO: 28) |
| ID # | 44) IC50 [M] | NO: 4) | IC50 | IC50 | IC50 |
| F0103387G05 | ND | ND | ND | ND | ND |
| F010302391 | 3.2E−08 | — | — | — | — |

—, no activity observed @ 7 μM;
ND, not determined

Part 3

| ID # | Tm [° C.] | Tagg [° C.] | aSEC | OD340 nm | AbM | Kabat |
|---|---|---|---|---|---|---|
| F0103387G05 | 74 | >70 | ok | ok | 81% | 76% |
| F010302391 | 73 | 53 | ok | ok | 87% | 82% |

ok, OD360 is acceptable

### Example 10

### Sequence Optimization of F0103464B09

Several PTM substitution libraries were generated based on the accelerated stress data summarized in Table 37 and screened as crude periplasmic extracts in binding FACS on human, rhesus and murine Nav1.7α. No substitution libraries were generated for M77 and N53 substitutions.

TABLE 37
Results accelerated stress experiments performed on F0103464B09 variants

| | | | | Modification |
|---|---|---|---|---|
| ID # | Description | Site | Stress condition | observed |
| F0103464B09 | F0103464B09-FLAG3- | NA | 1 week @ 45° C., ±1 | 0.1% increase |
| | HIS6 | | mg/mL in D-PBS | of pre-peak |
| | | | | (SE-HPLC) |
| F010301669 | F0103464B09-HIS6 | N53 | 4 weeks @ −20, 25 | >20% |
| | | | and 40° C. | |
| F010301669 | F0103464B09-HIS6 | M77 | 10 mM H₂O₂ for 3 h | >25% |
| | | | @ RT | |
| F010301669 | F0103464B09-HIS6 | N99 | 4 weeks @ −20, 25 | >20% |
| | | | and 40° C. | |
| F010302363 | F0103464B09(L11V, T24A, | NA | 1 week @ 45° C., ±1 | 0% increase |
| | T25S, S26H, R39Q, V40A, E44Q, | | mg/mL in D-PBS | of pre-peak |
| | N53E, N58Q, F62S, A63V, | | | (SE-HPLC) |
| | S68T, M77T, T79Y, R81Q, | | | |
| | S82aN, N82bS, K83R, G88A, | | | |
| | V89L, N99S)-FLAG3-HIS6 | | | |

NA, not applicable

N99S substitution resulted in a comparable or slightly improved binding profile compared to the parental reference F0103464B09 (Table 38) and was retained.

TABLE 38
Summary screening N99X & T100X substitution libraries

| | CHO Flp-In | | CHO Flp-In | | | |
|---|---|---|---|---|---|---|
| | huNav1.7α + | | rhNav1.7α + | | HEK Jmp-In | |
| | β1-β2-β3 | | β1-β2-β3 | | muNaV1.7α | |
| | (SEQ ID NO: 3) | | (SEQ ID NO: 4) | | (SEQ ID NO: 1) | |
| | Mean | SD | Mean | SD | Mean | SD |
| | MFI | MFI | MFI | MFI | MFI | MFI |
| Parental | 51339 | 12286 | 8778 | 2711 | 10524 | 1686 |
| reference | | | | | | |
| N99A | 67236 | 10630 | 611 | 18 | 6953 | 420 |
| N99C | 2148 | 693 | 528 | 6 | 930 | 25 |
| N99D | 17565 | 2934 | 520 | 30 | 929 | 0 |
| N99E | 12920 | 1629 | 528 | 6 | 938 | 1 |
| N99F | 65663 | 17996 | 516 | 6 | 938 | 1 |
| N99G | 35986 | 4476 | 530 | 8 | 956 | 21 |
| N99I | 42100 | 3304 | 540 | 6 | 946 | 7 |
| N99K | 38359 | 756 | 537 | 1 | 923 | 8 |
| N99L | 35233 | 3218 | 536 | 10 | 919 | 19 |
| N99M | 58378 | 435 | 523 | 0 | 936 | 15 |
| N99P | 612 | 16 | 554 | 20 | 912 | 12 |
| N99Q | 44147 | 8475 | 546 | 10 | 1487 | 123 |
| N99R | 54569 | 3670 | 572 | 0 | 975 | 5 |
| N99S | 68583 | 1345 | 4867 | 116 | 18128 | 1883 |
| N99T | 43801 | 75 | 839 | 22 | 12506 | 1026 |
| N99V | 48230 | 6360 | 552 | 6 | 952 | 16 |
| N99W | 102535 | 10470 | 530 | 2 | 3324 | 3361 |
| N99Y | 50347 | 70381 | 490 | 20 | 936 | 61 |
| T100A | 46487 | 4744 | 14750 | 239 | 13767 | 654 |
| T100D | 40000 | 8423 | 526 | 1 | 1105 | 37 |
| T100E | 41841 | 2122 | 518 | 33 | 973 | 23 |
| T100F | 30496 | 1300 | 533 | 30 | 997 | 19 |
| T100G | 23035 | 1891 | 2835 | 240 | 6429 | 120 |
| T100H | 53777 | 5512 | 689 | 4 | 5147 | 210 |
| T100I | 35094 | 8735 | 541 | 13 | 1311 | 24 |
| T100K | 24860 | 2730 | 583 | 15 | 8089 | 123 |
| T100L | 32189 | 9188 | 531 | 18 | 1251 | 175 |
| T100M | 38964 | 6535 | 585 | 34 | 2629 | 593 |
| T100P | 16711 | 1179 | 1581 | 206 | 2092 | 101 |
| T100Q | 39436 | 9907 | 650 | 53 | 4295 | 787 |
| T100R | 30391 | 2087 | 626 | 49 | 10230 | 957 |
| T100S | 50304 | 5618 | 11824 | 3287 | 11225 | 1855 |
| T100V | 27409 | 1370 | 564 | 13 | 1680 | 16 |
| T100Y | 25436 | 1212 | 526 | 16 | 1121 | 43 |

### Characterization of F0103464B09 Variants

In the first round, a large number of sequence optimization substitutions were explored. In the second round, eight different affinity maturation combinations (see Example 3) were explored for improved binding to rhesus Nav1.7α, combined with the remaining sequence optimization substitutions. The variants were compared in binding FACS on human, rhesus and murine Nav1.7α (muNav1.7α), in aSEC for possible for possible multimerization, in OD340 for insoluble aggregate formation, and in the thermal shift assay for Tm (Table 40). Table 35 summarizes the effects of the explored substitutions.

In the course of the sequence optimization process, subtle drops in binding to rhesus Nav1.7α were observed for the following substitutions: R39Q, A63V, T79Y, R81Q and N99S (Table 39). R39Q substitution also resulted in a subtle drop in binding to human Nav1.7α (Table 39). The combination of these, as present in the background in which the combinatorial affinity maturation substitutions were introduced, resulted in the complete abolishment of binding to rhesus Nav1.7α for the controls that do not carry any of the affinity maturation substitutions (variants F010302365, F010302366 and F010302368 in Table 40) and the same was observed for the variants combining the A28Q G54E substitutions. Less outspoken, none of the variants combining the A28Q G54E N58Q, S26H A28Q G54E N58Q or A28Q N53E G54S N58Q substitutions reached maximum binding levels to rhesus Nav1.7α (Table 40). A similar observation was made for the variants combining the S26H V33L N53E G54S substitutions, which also resulted in a drop in binding EC50 to human Nav1.7α. The three remaining combinations S26H N53E N58Q, S26H N53E G54S N58Q and S26H A28Q N53E N58Q were highly comparable for their binding to human and rhesus Nav1.7α (Table 40). The S26H N53E N58Q combination was then selected as it achieves the same binding improvements with one mutation less than the two others.

TABLE 39
Overview of F0103464B09 substitutions

| | huNav1.7α | rhNav1.7α | muNav1.7α | | | |
|---|---|---|---|---|---|---|
| | EC50 fold | EC50 fold | EC50 fold | Tm | aSEC | |
| | change | change | change | difference | behavior | |
| | compared | compared | compared | compared | compared | OD340 |
| | to | to | to | to | to | compared to |
| Substitution | reference | reference | reference | reference | reference | reference |
| L11V | = | = | − | −2 | = | = |
| (L11V, K83R, | | | | | | |
| V89L) | | | | | | |
| T24A | = | = | −2 | +1 | = | = |
| T25S | = | = | −2 | = | = | = |
| S26H | = | +3 | ND | +3 | = | = |
| | | | | (S26H, N53E, | | |
| | | | | N58Q) | | |
| A28Q | + | + | ND | ND | ND | ND |
| V33L | = | +3 | ND | ND | ND | ND |
| R39Q | − | − | −4 | +3 | = | = |
| V40A | = | = | = | −1 | = | = |
| E44Q | = | +3 | +3 | = | = | = |
| N53E | − | + | ND | +3 | = | = |
| | | | | (S26H, N53E, | | |
| | | | | N58Q) | | |
| G54E | = | + | ND | ND | ND | ND |
| G54S | = | + | ND | ND | ND | ND |
| N58Q | + | +2 | ND | +3 | = | = |
| | | | | (S26H, N53E, | | |
| | | | | N58Q) | | |
| F62S | = | = | = | −8 | = | = |
| A63V | = | − | −2 | −5 | = | = |
| | | | | +4 (F62S) | | |
| S68T | = | = | = | +2 | = | = |
| K76N | −2 | − | −4 | = | = | = |
| M77T | = | = | = | +2 | = | = |
| T79Y | = | − | = | +2 | = | = |
| R81Q | = | − | = | +2 | = | = |
| S82aN | = | = | = | = | = | = |
| N82bS | = | = | = | −3 | = | = |
| K83R | = | = | = | −2 | = | = |
| (L11V, K83R, | | | | | | |
| V89L) | | | | | | |
| G88A | = | = | = | −5 | = | |
| V89L | = | = | = | −2 | = | = |
| (L11V, K83R, | | | | | | |
| V89L) | | | | | | |
| L93N | − | −−− | −−− | −3 | = | = |
| N99S | = | − | = | ND | ND | ND |

=, activity is equivalent reference
−, lower activity than reference
+, higher activity to reference
ND, not determined

TABLE 40
Characterization of F0103464B09 variants

Part 1

| ID # | L11 | T24 | T25 | S26H | A28Q | V33L | R39 | V40 | E44 | N53E | G54E/S | N58Q | F62 |
|---|---|---|---|---|---|---|---|---|---|---|---|---|---|
| F010301868 | V | . | . | . | . | . | . | . | . | . | . | . | . |
| F010301869 | V | . | . | . | . | . | . | . | . | . | . | . | . |
| F010301870 | V | . | . | . | . | . | . | . | . | . | . | . | . |
| F010301871 | V | A | . | . | . | . | . | . | . | . | . | . | . |
| F010301872 | V | . | S | . | . | . | . | . | . | . | . | . | . |
| F010301873 | V | . | . | . | . | . | Q | . | . | . | . | . | . |
| F010301874 | V | . | . | . | . | . | . | A | . | . | . | . | . |
| F010301875 | V | . | . | . | . | . | . | . | . | . | . | . | S |
| F010301876 | V | . | . | . | . | . | . | . | . | . | . | . | . |
| F010301877 | V | . | . | . | . | . | . | . | . | . | . | . | . |
| F010301893 | V | . | . | . | . | . | . | . | Q | . | . | . | . |
| F010301932 | V | . | . | . | . | . | . | . | . | . | . | . | . |
| F010301933 | V | . | . | . | . | . | . | . | . | . | . | . | . |
| F010301934 | V | . | . | . | . | . | . | . | . | . | . | . | . |
| F010301935 | V | . | . | . | . | . | . | . | . | . | . | . | . |
| F010301936 | V | . | . | . | . | . | . | . | . | . | . | . | . |
| F010301937 | V | . | . | . | . | . | . | . | . | . | . | . | . |
| F010301938 | V | . | . | . | . | . | . | . | . | . | . | . | . |
| F010301939 | V | . | . | . | . | . | . | . | . | . | . | . | . |
| F010302333 | V | A | S | . | Q | . | . | A | Q | . | E | . | S |
| F010302334 | V | A | S | . | Q | . | . | A | Q | . | E | Q | S |
| F010302335 | V | A | S | . | Q | . | . | A | Q | E | S | Q | S |
| F010302336 | V | A | S | H | Q | . | . | A | Q | . | E | Q | S |
| F010302337 | V | A | S | H | Q | . | . | A | Q | E | . | Q | S |
| F010302338 | V | A | S | H | . | . | . | A | Q | E | S | Q | S |
| F010302339 | V | A | S | H | . | . | . | A | Q | E | . | Q | S |
| F010302340 | V | A | S | H | . | L | | A | Q | E | S | . | S |
| F010302341 | V | A | S | . | Q | . | Q | A | Q | . | E | . | S |
| F010302342 | V | A | S | . | Q | . | Q | A | Q | . | E | Q | S |
| F010302343 | V | A | S | . | Q | . | Q | A | Q | E | S | Q | S |
| F010302344 | V | A | S | H | Q | . | Q | A | Q | . | E | Q | S |
| F010302345 | V | A | S | H | Q | . | Q | A | Q | E | . | Q | S |
| F010302346 | V | A | S | H | . | . | Q | A | Q | E | S | Q | S |
| F010302347 | V | A | S | H | . | . | Q | A | Q | E | . | Q | S |
| F010302348 | V | A | S | H | . | L | Q | A | Q | E | S | . | S |
| F010302349 | V | A | S | . | Q | . | . | A | Q | . | E | . | S |
| F010302350 | V | A | S | . | Q | . | . | A | Q | . | E | Q | S |
| F010302351 | V | A | S | . | Q | . | . | A | Q | E | S | Q | S |
| F010302352 | V | A | S | H | Q | . | . | A | Q | . | E | Q | S |
| F010302353 | V | A | S | H | Q | . | . | A | Q | E | . | Q | S |
| F010302354 | V | A | S | H | . | . | . | A | Q | E | S | Q | S |
| F010302355 | V | A | S | H | . | . | . | A | Q | E | . | Q | S |
| F010302356 | V | A | S | H | . | L | . | A | Q | E | S | . | S |
| F010302357 | V | A | S | . | Q | . | Q | A | Q | . | E | . | S |
| F010302358 | V | A | S | . | Q | . | Q | A | Q | . | E | Q | S |
| F010302359 | V | A | S | . | Q | . | Q | A | Q | E | S | Q | S |
| F010302360 | V | A | S | H | Q | . | Q | A | Q | . | E | Q | S |
| F010302361 | V | A | S | H | Q | . | Q | A | Q | E | . | Q | S |
| F010302362 | V | A | S | H | . | . | Q | A | Q | E | S | Q | S |
| F010302363 | V | A | S | H | . | . | Q | A | Q | E | . | Q | S |
| F010302364 | V | A | S | H | . | L | Q | A | Q | E | S | . | S |
| F010302365 | V | A | S | . | . | . | . | A | Q | . | . | . | S |
| F010302366 | V | A | S | . | . | . | Q | A | Q | . | . | . | S |
| F010302368 | V | A | S | . | . | . | Q | A | Q | . | . | . | S |
| F0103464B09 | . | . | . | . | . | . | . | . | . | . | . | . | . |

Part 1

| ID # | A63 | S68 | K76 | M77 | T79 | R81 | S82a | N82b | K83 | G88 | V89 | L93 | N99 |
|---|---|---|---|---|---|---|---|---|---|---|---|---|---|
| F010301868 | . | T | . | . | Y | Q | N | S | R | A | L | . | . |
| F010301869 | . | T | . | T | Y | Q | N | S | R | A | L | . | . |
| F010301870 | . | T | . | . | Y | Q | N | S | R | A | L | N | . |
| F010301871 | . | T | . | . | Y | Q | N | S | R | A | L | . | . |
| F010301872 | . | T | . | . | Y | Q | N | S | R | A | L | . | . |
| F010301873 | . | T | . | . | Y | Q | N | S | R | A | L | . | . |
| F010301874 | . | T | . | . | Y | Q | N | S | R | A | L | . | . |
| F010301875 | . | T | . | . | Y | Q | N | S | R | A | L | . | . |
| F010301876 | V | T | . | . | Y | Q | N | S | R | A | L | . | . |
| F010301877 | . | T | N | . | Y | Q | N | S | R | A | L | . | . |
| F010301893 | . | T | . | . | Y | Q | N | S | R | A | L | . | . |
| F010301932 | . | . | . | . | . | . | . | . | R | . | L | . | . |
| F010301933 | . | T | . | . | . | . | . | . | R | . | L | . | . |
| F010301934 | . | . | . | T | . | . | . | . | R | . | L | . | . |
| F010301935 | . | . | . | . | Y | . | . | . | R | . | L | . | . |
| F010301936 | . | . | . | . | . | Q | . | . | R | . | L | . | . |
| F010301937 | . | . | . | . | . | . | N | . | R | . | L | . | . |
| F010301938 | . | . | . | . | . | . | . | S | R | . | L | . | . |
| F010301939 | . | . | . | . | . | . | . | . | R | A | L | . | . |
| F010302333 | . | . | . | T | Y | Q | N | S | R | A | L | . | S |
| F010302334 | . | T | . | T | Y | Q | N | S | R | A | L | . | S |
| F010302335 | . | T | . | T | Y | Q | N | S | R | A | L | . | S |
| F010302336 | . | T | . | T | Y | Q | N | S | R | A | L | . | S |
| F010302337 | . | T | . | T | Y | Q | N | S | R | A | L | . | S |
| F010302338 | . | T | . | T | Y | Q | N | S | R | A | L | . | S |
| F010302339 | . | T | . | T | Y | Q | N | S | R | A | L | . | S |
| F010302340 | . | T | . | T | Y | Q | N | S | R | A | L | . | S |
| F010302341 | . | T | . | T | Y | Q | N | S | R | A | L | . | S |
| F010302342 | . | T | . | T | Y | Q | N | S | R | A | L | . | S |
| F010302343 | . | T | . | T | Y | Q | N | S | R | A | L | . | S |
| F010302344 | . | T | . | T | Y | Q | N | S | R | A | L | . | S |
| F010302345 | . | T | . | T | Y | Q | N | S | R | A | L | . | S |
| F010302346 | . | T | . | T | Y | Q | N | S | R | A | L | . | S |
| F010302347 | . | T | . | T | Y | Q | N | S | R | A | L | . | S |
| F010302348 | . | T | . | T | Y | Q | N | S | R | A | L | . | S |
| F010302349 | V | T | . | T | Y | Q | N | S | R | A | L | . | S |
| F010302350 | V | T | . | T | Y | Q | N | S | R | A | L | . | S |
| F010302351 | V | T | . | T | Y | Q | N | S | R | A | L | . | S |
| F010302352 | V | T | . | T | Y | Q | N | S | R | A | L | . | S |
| F010302353 | V | T | . | T | Y | Q | N | S | R | A | L | . | S |
| F010302354 | V | T | . | T | Y | Q | N | S | R | A | L | . | S |
| F010302355 | V | T | . | T | Y | Q | N | S | R | A | L | . | S |
| F010302356 | V | T | . | T | Y | Q | N | S | R | A | L | . | S |
| F010302357 | V | T | . | T | Y | Q | N | S | R | A | L | . | S |
| F010302358 | V | T | . | T | Y | Q | N | S | R | A | L | . | S |
| F010302359 | V | T | . | T | Y | Q | N | S | R | A | L | . | S |
| F010302360 | V | T | . | T | Y | Q | N | S | R | A | L | . | S |
| F010302361 | V | T | . | T | Y | Q | N | S | R | A | L | . | S |
| F010302362 | V | T | . | T | Y | Q | N | S | R | A | L | . | S |
| F010302363 | V | T | . | T | Y | Q | N | S | R | A | L | . | S |
| F010302364 | V | T | . | T | Y | Q | N | S | R | A | L | . | S |
| F010302365 | . | T | . | T | Y | Q | N | S | R | A | L | . | S |
| F010302366 | . | T | . | T | Y | Q | N | S | R | A | L | . | S |
| F010302368 | V | T | . | T | Y | Q | N | S | R | A | L | . | S |
| F0103464B09 | . | . | . | . | . | . | . | . | . | . | . | . | . |

Part 2

| | | | | CHO | | |
|---|---|---|---|---|---|---|
| | | | CHO | FlpIn | | |
| | | | FlpIn | rhNav1.7α + | | |
| | | CHO FlpIn | rhNav1.7α + | β1-β2-β3 | | |
| | CHO FlpIn | huNav1.7α + | β1-β2- β3 | (SEQ ID | | |
| | huNav1.7α + | β1-β2-β3 | (SEQ ID | NO: 4) | HEK JmpIn | HEK JmpIn |
| | β1-β2-β3 | (SEQ ID | NO: 3) | Bmax | muNav1.70α | muNav1.7α |
| | (SEQ ID NO: 3) | NO: 3) | EC50 | (including | (SEQ ID NO: | (SEQ ID NO: |
| ID # | EC50 [M] | Bmax | [M] | affmat mutations) | 45) EC50 [M] | 453) Bmax |
| F010301868 | 6.3E−09 | 98% | 6.2E−08 | ND | 1.1E−08 | ND |
| F010301869 | 7.6E−09 | 100% | 9.6E−08 | ND | 1.7E−08 | ND |
| F010301870 | 1.0E−08 | 100% | — | ND | — | ND |
| F010301871 | 5.8E−09 | 100% | 3.7E−08 | ND | 2.4E−08 | ND |
| F010301872 | 6.8E−09 | 100% | 5.7E−08 | ND | 2.1E−08 | ND |
| F010301873 | 9.0E−09 | 100% | 1.1E−07 | ND | 4.2E−08 | ND |
| F010301874 | 6.5E−09 | 100% | 5.5E−08 | ND | 1.8E−08 | ND |
| F010301875 | 6.4E−09 | 100% | 5.2E−08 | ND | 1.4E−08 | ND |
| F010301876 | 6.3E−09 | 100% | 1.1E−07 | ND | 2.3E−08 | ND |
| F010301877 | 1.3E−08 | 100% | 9.6E−08 | ND | 3.9E−08 | ND |
| F010301893 | 5.8E−09 | 100% | 2.1E−08 | ND | 3.6E−09 | ND |
| F010301932 | 4.3E−09 | 94% | 1.7E−08 | ND | 4.8E−09 | ND |
| F010301933 | 4.4E−09 | 94% | 2.1E−08 | ND | 5.3E−09 | ND |
| F010301934 | 4.3E−09 | 96% | 2.1E−08 | ND | 4.5E−09 | ND |
| F010301935 | 3.8E−09 | 94% | 2.7E−08 | ND | 5.5E−09 | ND |
| F010301936 | 4.4E−09 | 95% | 2.6E−08 | ND | 4.8E−09 | ND |
| F010301937 | 4.3E−09 | 95% | 2.0E−08 | ND | 4.8E−09 | ND |
| F010301938 | 4.8E−09 | 97% | 2.0E−08 | ND | 4.7E−09 | ND |
| F010301939 | 5.1E−09 | 96% | 2.2E−08 | ND | 6.3E−09 | ND |
| F010302333 | 4.1E−09 | 100% | — | — | ND | ND |
| F010302334 | 4.0E−09 | 100% | 1.1E−08 | 70% | ND | ND |
| F010302335 | 4.1E−09 | 100% | 1.3E−08 | 84% | ND | ND |
| F010302336 | 3.6E−09 | 100% | 9.7E−09 | 79% | ND | ND |
| F010302337 | 3.9E−09 | 100% | 6.8E−09 | 100% | ND | 74% |
| F010302338 | 3.4E−09 | 100% | 7.9E−09 | 100% | ND | ND |
| F010302339 | 4.3E−09 | 100% | 6.8E−09 | 100% | 8.7E−09 | 61% |
| F010302340 | 4.6E−09 | 100% | 1.3E−08 | 96% | ND | ND |
| F010302341 | 5.3E−09 | 100% | — | — | ND | ND |
| F010302342 | 4.0E−09 | 100% | 1.6E−08 | 61% | ND | ND |
| F010302343 | 6.4E−09 | 100% | 1.8E−08 | 84% | ND | ND |
| F010302344 | 4.3E−09 | 100% | 1.3E−08 | 78% | ND | ND |
| F010302345 | 4.9E−09 | 100% | 1.2E−08 | 96% | ND | ND |
| F010302346 | 5.0E−09 | 100% | 1.2E−08 | 99% | ND | ND |
| F010302347 | 4.6E−09 | 100% | 9.6E−09 | 100% | 1.4E−08 | 48% |
| F010302348 | 6.7E−09 | 100% | 1.4E−08 | 95% | ND | ND |
| F010302349 | 4.4E−09 | 100% | — | — | ND | ND |
| F010302350 | 3.5E−09 | 100% | 1.5E−08 | 46% | ND | ND |
| F010302351 | 5.6E−09 | 100% | 1.5E−08 | 72% | ND | ND |
| F010302352 | 3.6E−09 | 100% | 1.1E−08 | 60% | ND | ND |
| F010302353 | 4.2E−09 | 100% | 7.9E−09 | 93% | ND | ND |
| F010302354 | 4.2E−09 | 100% | 7.9E−09 | 96% | ND | ND |
| F010302355 | 3.5E−09 | 100% | 6.8E−09 | 97% | 2.1E−08 | 30% |
| F010302356 | 5.3E−09 | 100% | 1.2E−08 | 81% | ND | ND |
| F010302357 | 5.3E−09 | 100% | — | — | ND | ND |
| F010302358 | 4.5E−09 | 100% | 3.7E−08 | 37% | ND | ND |
| F010302359 | 6.4E−09 | 100% | 2.4E−08 | 66% | ND | ND |
| F010302360 | 3.5E−09 | 100% | 1.5E−08 | 58% | ND | ND |
| F010302361 | 4.8E−09 | 100% | 1.1E−08 | 92% | ND | ND |
| F010302362 | 5.4E−09 | 100% | 1.4E−08 | 89% | ND | ND |
| F010302363 | 4.2E−09 | 100% | 9.3E−09 | 96% | 4.8E−08 | 18% |
| F010302364 | 7.2E−09 | 100% | 1.7E−08 | 74% | ND | ND |
| F010302365 | 3.1E−09 | 100% | — | — | ND | ND |
| F010302366 | 3.7E−09 | 100% | — | — | ND | ND |
| F010302368 | 3.9E−09 | 100% | — | — | ND | ND |
| F0103464B09 | 5.2E−09 | 99% | 1.8E−08 | 33% | 6.3E−09 | 74% |

ND, not determined

Part 3

| ID # | Tm [° C.] | aSEC | OD340 nm |
|---|---|---|---|
| F010301868 | 66 | ok | ok |
| F010301869 | 67 | ok | ok |
| F010301870 | 63 | ok | ok |
| F010301871 | 67 | ok | ok |
| F010301872 | 66 | ok | ok |
| F010301873 | 69 | ok | ok |
| F010301874 | 65 | ok | ok |
| F010301875 | 58 | ok | ok |
| F010301876 | 61 | ok | ok |
| F010301877 | 66 | ok | ok |
| F010301893 | 66 | ok | ok |
| F010301932 | 64 | ok | ok |
| F010301933 | 66 | ok | ok |
| F010301934 | 66 | ok | ok |
| F010301935 | 66 | ok | ok |
| F010301936 | 66 | ok | ok |
| F010301937 | 64 | ok | ok |
| F010301938 | 61 | ok | ok |
| F010301939 | 59 | ok | ok |
| F010302333 | ND | ND | ND |
| F010302334 | ND | ND | ND |
| F010302335 | ND | ND | ND |
| F010302336 | ND | ND | ND |
| F010302337 | ND | ND | ND |
| F010302338 | ND | ND | ND |
| F010302339 | 63 | ok | ok |
| F010302340 | ND | ND | ND |
| F010302341 | ND | ND | ND |
| F010302342 | ND | ND | ND |
| F010302343 | ND | ND | ND |
| F010302344 | ND | ND | ND |
| F010302345 | ND | ND | ND |
| F010302346 | ND | ND | ND |
| F010302347 | 66 | ok | ok |
| F010302348 | ND | ND | ND |
| F010302349 | ND | ND | ND |
| F010302350 | ND | ND | ND |
| F010302351 | ND | ND | ND |
| F010302352 | ND | ND | ND |
| F010302353 | ND | ND | ND |
| F010302354 | ND | ND | ND |
| F010302355 | 66 | ok | ok |
| F010302356 | ND | ND | ND |
| F010302357 | ND | ND | ND |
| F010302358 | ND | ND | ND |
| F010302359 | ND | ND | ND |
| F010302360 | ND | ND | ND |
| F010302361 | ND | ND | ND |
| F010302362 | ND | ND | ND |
| F010302363 | 70 | ok | ok |
| F010302364 | ND | ND | ND |
| F010302365 | 60 | ok | ok |
| F010302366 | 63 | ok | ok |
| F010302368 | 66 | ok | ok |
| F0103464B09 | 66 | ok | ok |

ND, not determined

### Selection of an F0103464B09 Sequence Optimization Variant

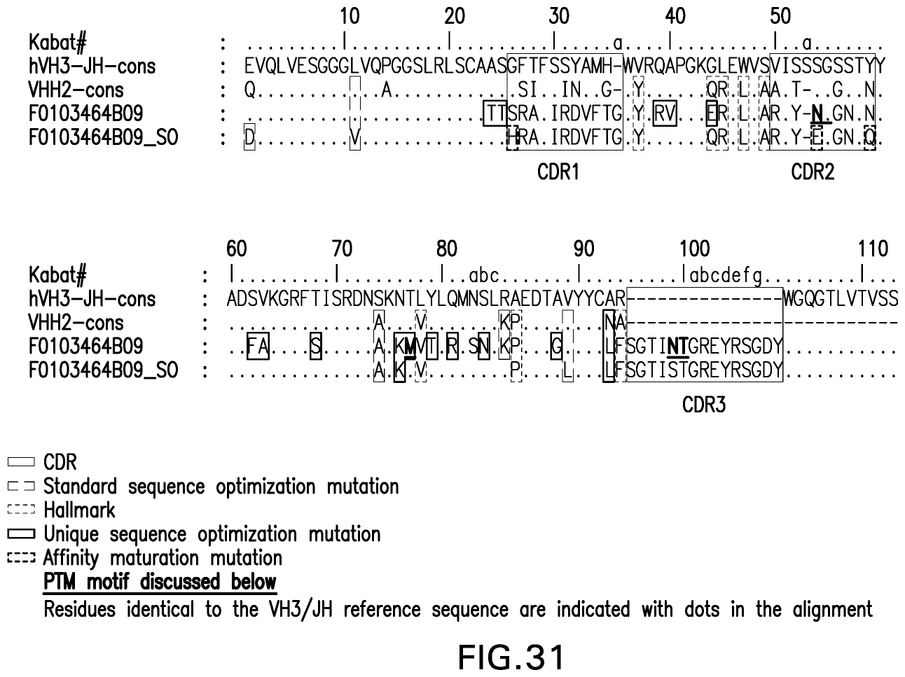

Variant F010302363 was selected as the final sequence optimization variant of F0103464B09 (see F0103464B09_SO in FIG. 31). It boasts a strongly improved binding on rhesus Nav1.7α, reduced binding to muNav1.7a, as well as comparable aSEC and OD340 nm behavior and an improved thermal stability (Table 41). In vitro electrophysiology experiments confirmed the low nM potency on huNav1.7α and rhNav1.7α and selectivity over Nav1.4α, Nav1.5α, and Nav1.6α. All PTM liabilities (Table 37) were successfully substituted. Cell-free fermentation expression titers at CMC of the corresponding monovalent tagless variant F01032390 in P. pastoris were 2.0 g/L.

TABLE 41
Sequence optimization variant of F0103464B09
Part 1

| | CHO FlpIn | | CHO FlpIn | | |
|---|---|---|---|---|---|
| | huNav1.7α + | CHO FlpIn | rhNav1.7α + | | |
| | β1-β2-β3 | rhNav1.7α + | β1- β2-β3 | HEK JmpIn | HEK JmpIn |
| | (SEQ ID | β1- β2-β3 | (SEQ ID NO: | muNav1.7α | muNav1.7α |
| | NO: 3) EC50 | (SEQ ID NO: | 4) Bmax | (SEQ ID NO: | (SEQ ID NO: |
| ID # | [M] | 4) EC50 [M] | (including | 45) EC50 [M] | 45) Bmax |
| F0103464B09 | 5.2E−09 | 1.8E−08 | 33% | 6.3E−09 | 74% |
| F010302363 | 4.2E−09 | 9.3E−09 | 96% | 4.8E−08 | 18% |

—, no activity observed @ 7 μM

Part 2

| | HEK | HEK | HEK | HEK | HEK |
|---|---|---|---|---|---|
| | huNav1.7α + | rhNav1.7α + | huNav1.4 | huNav1.5 | huNav1.6α |
| | β1 (SEQ ID | β1-β2-β3 | (SEQ ID | (SEQ ID | (SEQ ID |
| | NO: 44) IC50 | (SEQ ID NO: | NO: 26) | NO: 27) | NO: 28) |
| ID # | [M | 4) IC50 [M] | IC50 [M] | IC50 [M] | IC50 [M] |
| F0103464B09 | ND | ND | ND | ND | ND |
| F010302363 | 1.1E−08 | 3.6E−08 | — | — | — |

—, no activity observed @ 7 μM;
ND, not determined

Part 3

| | | | | OD340 | | |
|---|---|---|---|---|---|---|
| ID # | Tm [° C.] | Tagg [° C.] | aSEC | nm | AbM | Kabat |
| F0103464B09 | 66 | Inconclusive | ok | ok | 72% | 69% |
| F010302363 | 70 | 71 | ok | ok | 85% | 80% |

ok, acceptable

### Example 11

### Identification of Anti-Navβ Subunit ISVDs

The aim of this campaign was to identify lead candidates that bind to different, non-overlapping epitopes compared to previously identified extracellular Nav1.7α binders (see previous examples). To this end, a selection and screening strategy was designed to identify lead candidates that would be able to bind in an avid fashion, when combined with a previously identified extracellular Nav1.7α binding ISVD.

Different immune repertoires were cloned downstream of an anchor building block [(F103275B05(N93R), a rhNav1.7α cross-reactive variant] separated by a long 50GS linker, resulting in bivalent phage display libraries.

Selections using high quality proteoliposome (PL) preparations or cell lines as antigen were performed on bivalent libraries derived from immunization schedules in which the animals first were repeatedly administered with different forms of full-length DNA, followed by up to four administrations with PL or membrane extract (ME), followed again by multiple administrations with different forms of full-length DNA. Crude periplasmic extracts containing bivalent ISVDs enriched by the selection process, were screened in binding FACS and competition FACS on different cell lines. Table 42 summarizes the screening data of five lead ISVD candidates F0103478E09, F0103492E09, F0103495F09, F0103500E03 and F0103505D08 (for the screening each F0103478E09, F0103492E09, F0103495F09, F0103500E03 and F0103505D08 was linked at the N-terminus to the C-terminus of an F103275B05(N93R)-50GS moiety to form a bivalent ISVD) for which the totality of the data in comparison to a control (bivalent ISVD F010300702 comprising an irrelevant anti-RSV building block linked at the N-terminus to the C-terminus of an F103275B05(N93R)-50GS moiety) suggests that they bind in an avid fashion to Nav1.7α:
selective binding on hu & rhNav1.7α in HEK & CHO at higher levels than the average of control F010300702
remaining binding after competition with anchor building block at higher levels than the average of control F010300702

Sequence analysis revealed that these lead candidates are unrelated and belong to different ISVD families (last column of (Table 42). Most of these lead candidates and/or related family members with high sequence similarity were identified multiple times throughout different selection and screening campaigns. Further characterization revealed that these lead candidates did not bind to Nav1.7α but instead were Navβ1 or Navβ2 binders.

TABLE 42
Overview binding and competition FACS screening of selected Navβ binders
Part 1

| | | | CHO | CHO |
|---|---|---|---|---|
| | | | Flp-In | Flp-In |
| | | | huNav1.5- | huNav1.5- |
| | CHO | CHO | β1-β2-β3 in | β1-β2-β3 in |
| | Flp-In | Flp-In | competition | competition |
| | huNav1.7- | huNav1.5- | vs. 1 μM | vs. 1 μM |
| | β1-β2-β3 | β1-β2-β3 | IRR00092 | F010300703 |
| ID # | (MFI) | (MFI) | (MFI) | (MFI) |
| F0103PMP478E09 | 82782 | 1079 | 2065 | 833 |
| F0103PMP492E09 | 40185 | 559 | 1860 | 825 |
| F0103PMP495F09 | 35600 | 483 | 930 | 645 |
| F0103PMP500E03 | 22450 | 521 | 975 | 852 |
| F0103PMP505D08 | 106009 | 891 | 964 | 845 |
| F010300702 (n = 15) | 1567 | 662 | 684 | 653 |

Part 2

| | CHO | | CHO | CHO |
|---|---|---|---|---|
| | Flp-In | CHO | Flp-In | Flp-In |
| | huNav1.7- | Flp-In | rhNav1.7- | rhNav1.7- |
| | β1-β2-β3 in | huNav1.7- | β1-β2-β3 in | β1-β2-β3 in |
| | competition | β1-β2-β3 in | competition | competition |
| | vs. 1 μM | competition | vs. 1 μM | vs. 1 μM |
| | IRR00092 | vs. 1 μM | IRR00092 | F010300703 |
| ID # | (MFI) | [symbol] | (MFI) | (MFI) |
| F0103PMP478E09 | 136552 | 28773 | 139028 | 60635 |
| F0103PMP492E09 | 104334 | 11981 | 111433 | 25186 |
| F0103PMP495F09 | 93301 | 7161 | 90368 | 6280 |
| F0103PMP500E03 | 33732 | 5376 | 96947 | 21477 |
| F0103PMP505D08 | 95366 | 9240 | 108235 | 22537 |
| F010300702 (n = 15) | 4120 | 812 | 5168 | 721 |

Part 3

| | | HEK | | HEK | |
|---|---|---|---|---|---|
| | | Flp-In | HEK | Flp-In | |
| | HEK293 | huNav1.5- | Flp-In | huNav157chim14- | |
| | Flp-In | β1-β2-β3 | huNav157chim14 | β1-β2-β3 | |
| ID # | (MFI) | (MFI) | (MFI) | (MFI) | Family |
| F0103PMP478E09 | 1052 | 877 | 799 | 1234 | 1037 |
| F0103PMP492E09 | 1205 | 1656 | 58377 | 132919 | 1044 |
| F0103PMP495F09 | 1090 | 976 | 45146 | 127724 | 1040 |
| F0103PMP500E03 | 1084 | 1106 | 47300 | 87229 | 1042 |
| F0103PMP505D08 | 1041 | 1198 | 78390 | 136037 | 1053 |
| F010300702 (n = 15) | 1132 | 946 | 27719 | 15389 | NA |

NA, not applicable [symbol]
indicates data missing or illegible when filed

### Binding Characterization Monovalent β-Subunit Binders

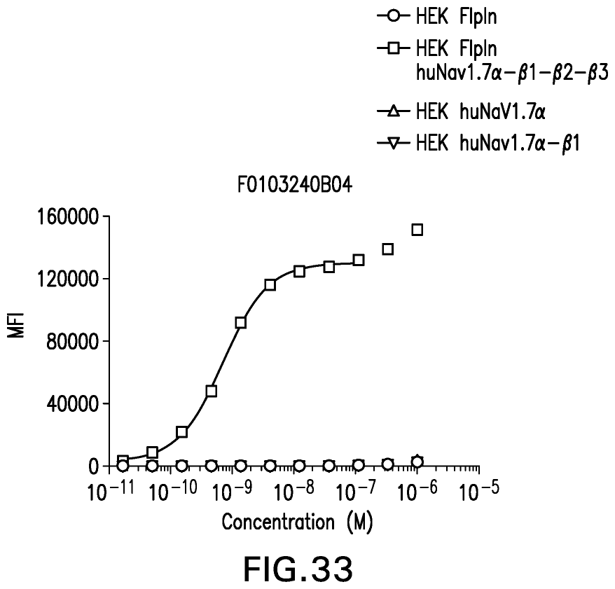

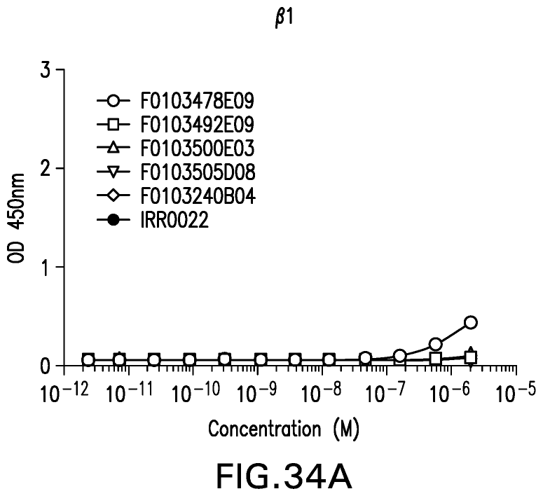

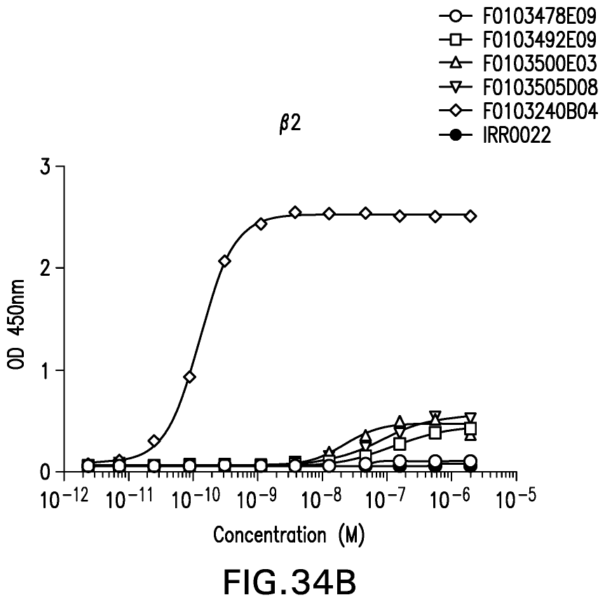

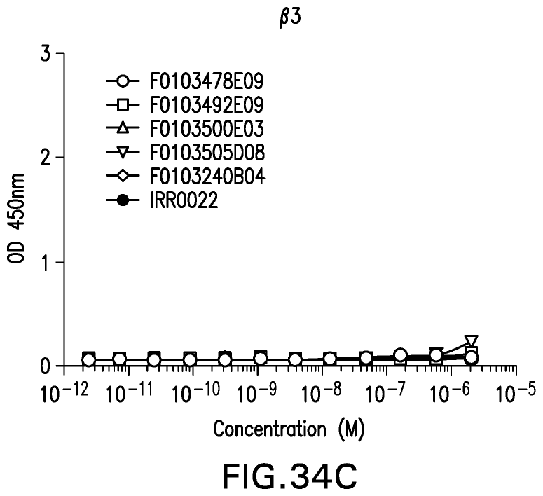

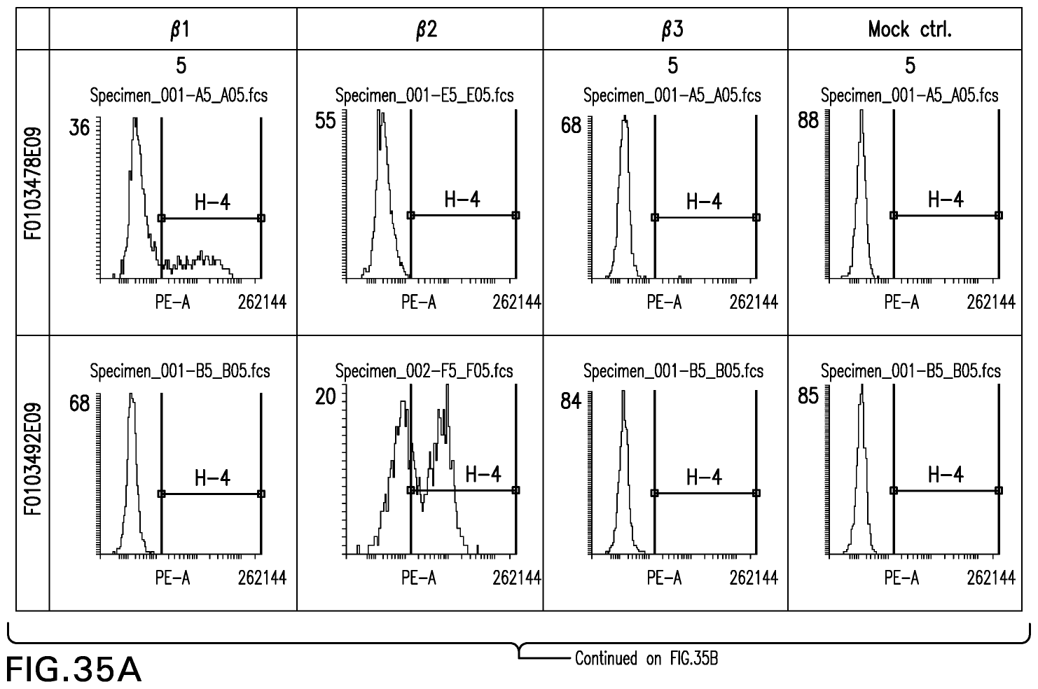

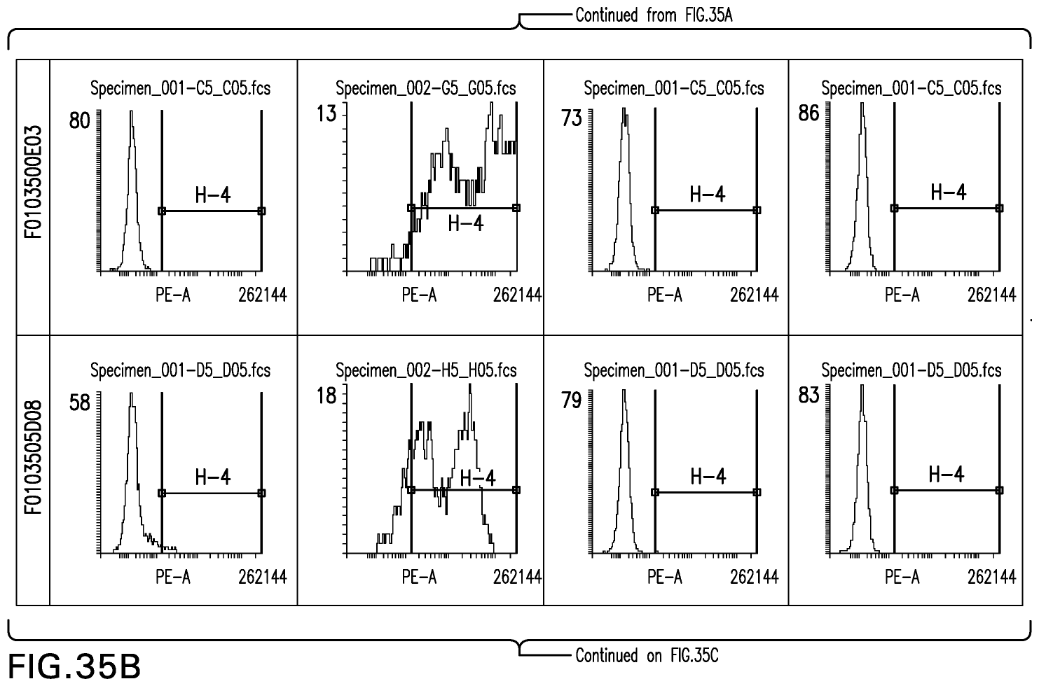

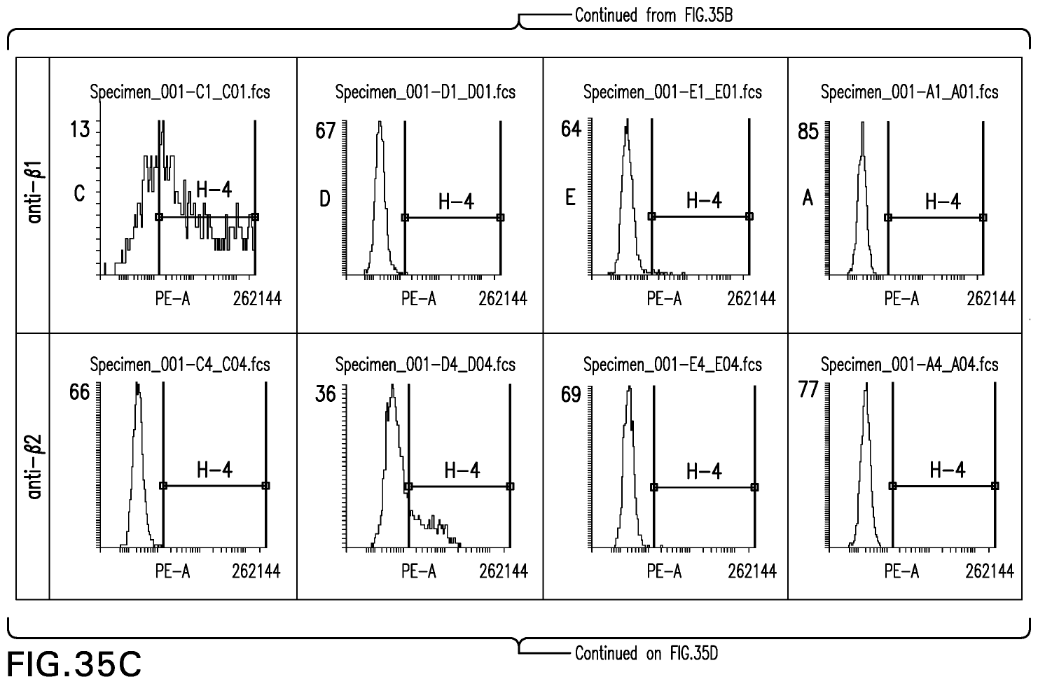

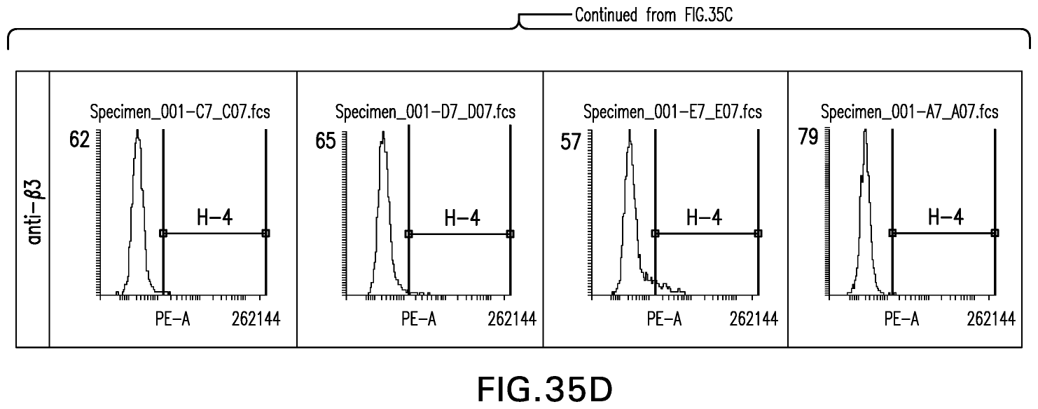

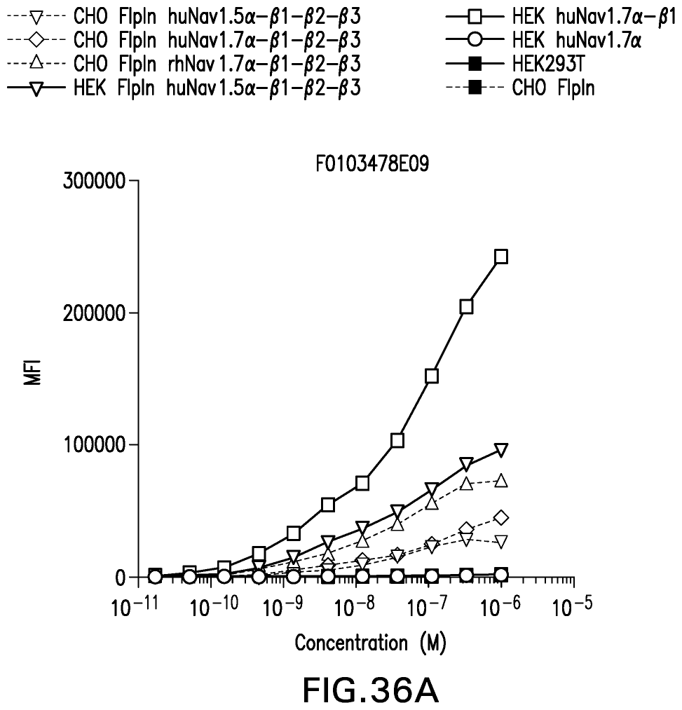

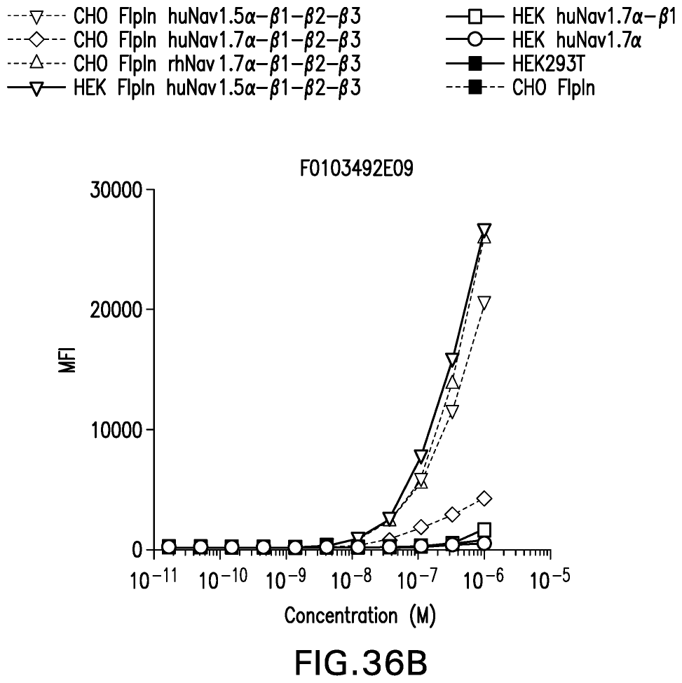

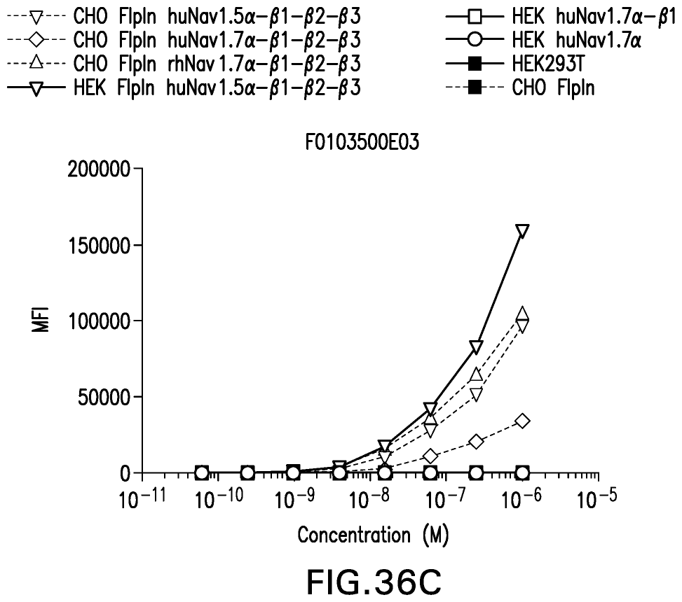

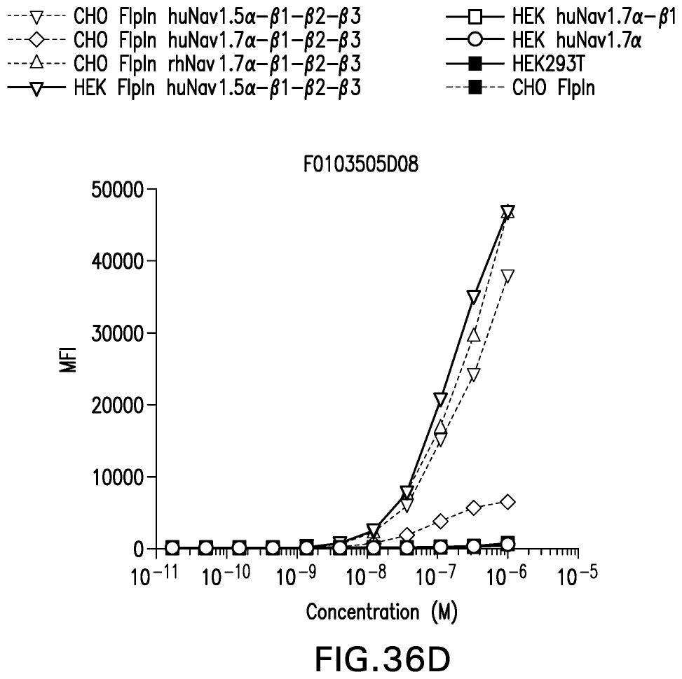

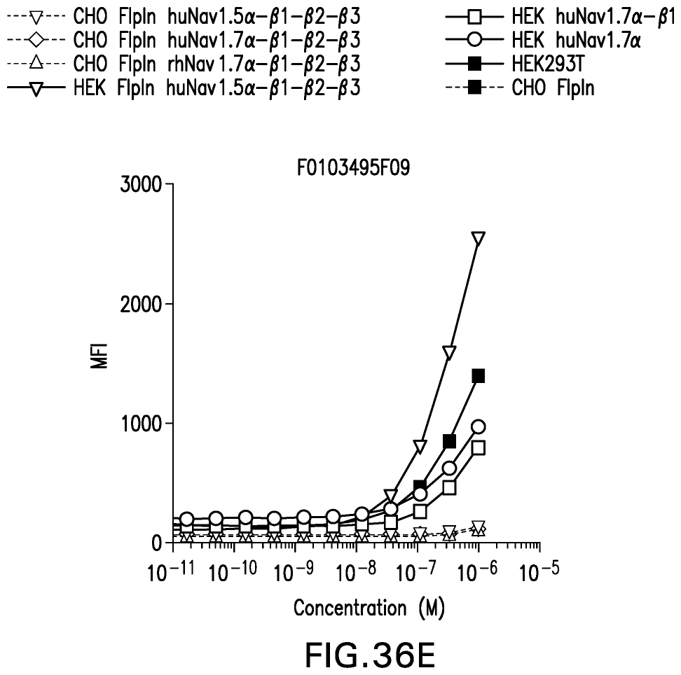

ISVD F0103240B04 was identified by means of binding ELISA as a candidate Navβ2 binder. Binding FACS (FIG. 33) and binding ELISA (FIG. 34B) experiments with purified monovalent protein suggest that F0103240B04 is indeed a potent Navβ2 binder. Five ISVDs, F0103478E09, F0103492E09, F0103495F09, F0103500E03 and F0103505D08, identified by binding and competition FACS (Table 42) were further characterized as purified monovalent protein. The combined data from the binding ELISA (FIGS. 34A-34C) and binding FACS experiments (FIGS. 35A-35D and FIGS. 36A-36E) suggest that F0103478E09 is a weak Navβ1 binder and that F0103492E09, F0103500E03, and F0103505D08 are weak Navβ2 binders. F0103495F09 was not evaluated as purified monovalent protein in the binding ELISA or binding FACS experiments using transiently transfected cells because binding FACS experiments using stable cell lines suggest that it recognizes a HEK293-specific cell background marker (See FIG. 36E). Additional competition FACS experiments with Nav1.7α-Navβ-subunit bispecific ISVDs; however, classify F103495F09 as a weak Navβ1 binder, similar to F0103478E09.

### Binding ELISA

In general, 10 μg/mL of HEK huNav1.7α-Navβ1 (huNav1.7-(31) expressing cells and HEK293T null ME cells were coated in bicarbonate buffer (pH9.6) overnight at 4° C. in 384-well HB Spectraplate (catalog #6007500, Perkin Elmer). Wells were blocked with 4% Marvel in PBS. After addition of periplasmic extracts (either pen (1/5) or purified ISVD) diluted in 2% Marvel (Premier Foods Group, St Albans, UK) in PBS, FLAG3-tagged ISVD binding was detected using a mouse anti-Flag-HRP conjugate (catalog #A8592-1MG, Sigma) and a subsequent enzymatic reaction in the presence of the substrate esTMB (3,3′,5,5′-tetramentylbenzidine) (catalog ##esTMB, SDT). Plates were read out on a MultiSkan device (ThermoFisher Scientific) at OD450. EC50 values were calculated using four-parameter logistic curves in GraphPad Prism7.

Alternatively, 3 μg/mL of HEK huNav1.7α-Navβ1-Navβ2-Navβ3 (huNav1.7-β1-β2-β3) cl. 11 PL was used as coated antigen in combination with detection of CMYC3-tagged ISVDs by mouse anti-c-myc biotin conjugate (catalog #MCA2200B Serotec) followed by extravidin-HRP conjugate (catalog #E2886, Sigma-Aldrich).

### Example 12

### Nav1.7α-Navβ Bispecific ISVDs

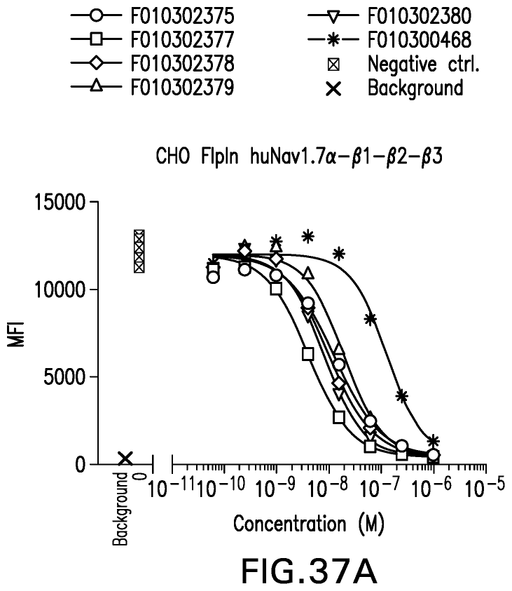

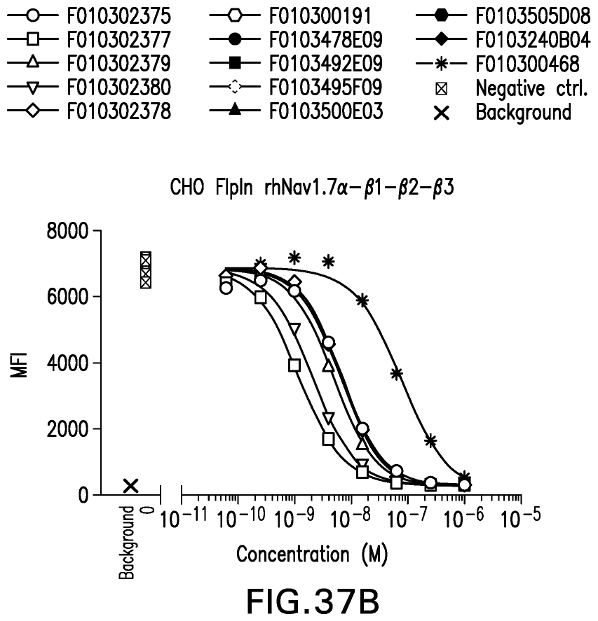

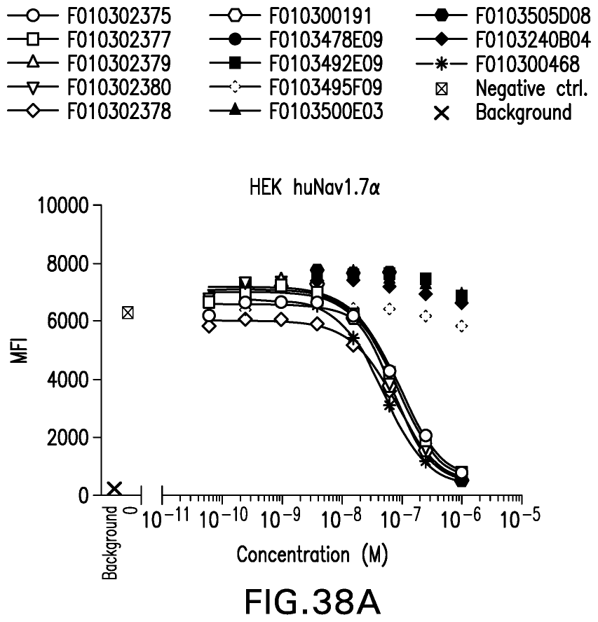

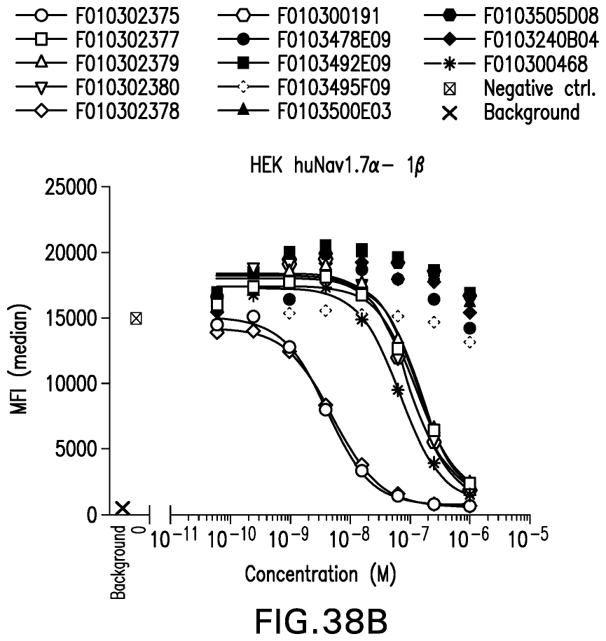

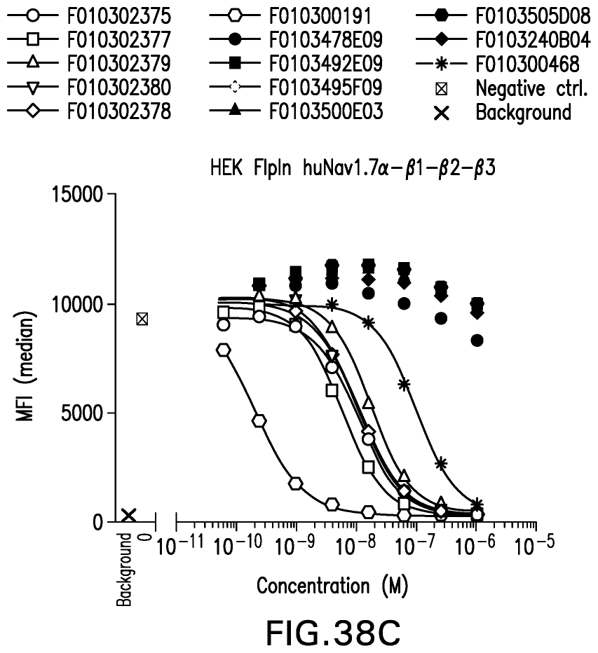

Bispecific leads were generated, fusing different anti-Navβ ISVDs to the C-terminus of the rhesus cross-reactive anti-Nav1.7α ISVD F103275B05(N93R) by means of a long flexible 50GS linker. The bispecifics were evaluated for their ability to compete for binding with the monovalent F0103275B05(N73R) variant to Nav1.7α in FACS experiments on different cell lines. The data shown in Table 43, FIGS. 37A-37B, and FIGS. 38A-38C reveals 10-to 1000-fold improved competition FACS IC50 values compared to the monovalent F0103275B05(N73R) control (F010300468 in table). This holds true for both Navfllbinders and Navβ2 binders on cell lines expressing the relevant counterparts. Also, stronger Navβ binders bring about greater IC50 improvements to the respective bispecifics. The monovalent Navβ binders were not able to displace F0103275B05(N73R) from Nav1.7α by themselves (FIGS. 38A-38C).

TABLE 43
Summary competition FACS of anti-Nav1.7α-Navβ bispecific ISVDs
Part 1

| | | | HEK | HEK |
|---|---|---|---|---|
| | | | huNav1.7α | huNav1.7α-β1 |
| | | Classification | vs. EC25 of | vs. EC25 of |
| | | 2^{nd} | F103275B05(N93R) | F103275B05(N93R) |
| ID # | Description | ISVD | IC50 [M] | IC50 [M] |
| F010302375 | F0103275B05(E1D, N93R)-50GS- | weak | 1.1 × 10⁻⁰⁷ | 4.3 × 10⁻⁰⁹ |
| | F0103478E09(L108Q) + FLAG3- | Navβ1 | | |
| | HIS6 | binder | | |
| F010302377 | F0103275B05(E1D, N93R)-50GS- | weak | 8.6 × 10⁻⁰⁸ | 1.4 × 10⁻⁰⁷ |
| | F0103492E09 + FLAG3-HIS6 | Navβ2 | | |
| | | binder | | |
| F010302378 | F0103275B05(E1D, N93R)-50GS- | weak | 8.0 × 10⁻⁰⁸ | 5.2 × 10⁻⁰⁹ |
| | F0103495F09 + FLAG3-HIS6 | Navβ1 | | |
| | | binder | | |
| F010302379 | F0103275B05(E1D, N93R)-50GS- | weak | 8.5 × 10⁻⁰⁸ | 1.5 × 10⁻⁰⁷ |
| | F0103500E03(P14A, L108Q) + | Navβ2 | | |
| | FLAG3-HIS6 | binder | | |
| F010302380 | F0103275B05(E1D, N93R)-50GS- | weak | 6.7 × 10⁻⁰⁸ | 1.1 × 10⁻⁰⁷ |
| | F0103505D08(L108Q) + FLAG3- | Navβ2 | | |
| | HIS6 | binder | | |
| F010300191 | F0103275B05-50GS-F0103240B04 + | strong | 6.7 × 10⁻⁰⁸ | 1.1 × 10⁻⁰⁷ |
| | FLAG3-HIS6 | Navβ2 | | |
| | | binder | | |
| F010300468 | F0103275B05(N93R) + FLAG3-HIS6 | NA | 4.9 × 10⁻⁰⁸ | 6.7 × 10⁻⁰⁸ |

Part 2

| | | | HEK | CHO | CHO |
|---|---|---|---|---|---|
| | | | FlpIn | FlpIn | FlpIn |
| | | | huNav1.7α- | huNav1.7α- | rhNav1.7α- |
| | | | β1-β2-β3 vs. | β1-β2-β3 vs. | β1-β2-β3 vs. |
| | | Classification | EC25 of | EC25 of | EC40 of |
| | | 2^{nd} | F103275B05(N93R) | F103275B05(N93R) | F103275B05(N93R) |
| ID # | Description | ISVD | IC50 [M] | IC50 [M] | IC50 [M] |
| F010302375 | F0103275B05(E1D, N93R)- | weak | 1.0 × 10⁻⁰⁸ | 1.2 × 10⁻⁰⁸ | 6.5 × 10⁻⁰⁹ |
| | 50GS-F0103478E09(L108Q) + | Navβ1 | | | |
| | FLAG3-HIS6 | binder | | | |
| F010302377 | F0103275B05(E1D, N93R)- | weak | 5.2 × 10⁻⁰⁹ | 4.1 × 10⁻⁰⁹ | 1.2 × 10⁻⁰⁹ |
| | 50GS-F0103492E09-FLAG3 + | Navβ2 | | | |
| | HIS6 | binder | | | |
| F010302378 | F0103275B05(E1D, N93R)- | weak | 1.2 × 10⁻⁰⁸ | 1.0 × 10⁻⁰⁸ | 6.6 × 10⁻⁰⁹ |
| | 50GS-F0103495F09-FLAG3 + | Navβ1 | | | |
| | HIS6 | binder | | | |
| F010302379 | F0103275B05(E1D, N93R)- | weak | 1.6 × 10⁻⁰⁸ | 1.9 × 10⁻⁰⁸ | 4.8 × 10⁻⁰⁹ |
| | 50GS- | Navβ2 | | | |
| | F0103500E03(P14A, L108Q)- | binder | | | |
| | FLAG3 + HIS6 | | | | |
| F010302380 | F0103275B05(E1D, N93R)- | weak | 8.7 × 10⁻⁰⁹ | 8.1 × 10⁻⁰⁹ | 2.0 × 10⁻⁰⁹ |
| | 50GS- | Navβ2 | | | |
| | F0103505D08(L108Q)- | binder | | | |
| | FLAG3 + HIS6 | | | | |
| F010300191 | F0103275B05-50GS- | strong | 1.0 × 10⁻¹⁰ | ND | ND |
| | F0103240B04-FLAG3 + | Navβ2 | | | |
| | HIS6 | binder | | | |
| F010300468 | F103275B05(N93R) + | NA | 9.7 × 10⁻⁰⁸ | 1.3 × 10⁻⁰⁷ | 7.7 × 10⁻⁰⁸ |
| | FLAG3-HIS6 | | | | |

NA, not applicable
NA, not applicable;
ND, not determined

### Example 13

This example shows that in vivo performance may be enhanced by half-life extension (HLE), which may be particularly useful in therapeutic formats for chronic pain indications. Two types of HLE formats were evaluated: fusion to (i) the anti-SA building block ALB23002 or to (ii) huFc.

A number of pilot experiments were performed with the rhesus cross-reactive affinity maturation variant F010300659 of F0103275B05. The addition of ALB23002 to the C-terminus of F010300659 separated by a flexible GlySer linker resulted in a two- to five-fold drop in binding competition (Table 44) and functional (Table 45 and FIG. 7A) potency. In the presence of a saturating concentration of human SA, an additional two- to ten-fold reduction in potency was observed which appeared to be more pronounced for the shorter 9GS compared to the longer 35GS linker construct.

TABLE 44
Summary competition FACS of ALB23002 HLE Nav1.7 ISVDs in presence/absence of human SA

| | | | CHO FlpIn | | | CHO FlpIn | |
|---|---|---|---|---|---|---|---|
| | | | huNav1.7-β1-β2- | | | rhNav1.7-β1-β2- | |
| | | CHO FlpIn | β3 vs. EC25 | | CHO FlpIn | β3 vs. EC25 | |
| | | huNav1.7-β1-β2- | of 275B05(N93R) | | rhNav1.7-β1-β2- | of 275B05(N93R) | |
| | | β3 vs. EC25 | IC50 [M] + | Human | β3 vs. EC25 | IC50 [M] + | Human |
| | | of 275B05(N93R) | 50 μM | SA | of 275B05(N93R) | 50 μM | SA |
| ID # | Description | IC50 [M] | human SA | ratio | IC50 [M] | human SA | ratio |
| F010301452 | F0103275B05(S27P, | 3.0 × 10⁻⁰⁸ | ND | NA | 1.6 × 10⁻⁰⁸ | ND | NA |
| | I28V, S50Y, N53P, | | | | | | |
| | G55W, S56D, T57W, | | | | | | |
| | N93R, A94W) | | | | | | |
| F010301465 | F0103275B05(E1D, | 9.8 × 10⁻⁰⁸ | 4.8 × 10⁻⁰⁷ | 5 | 6.0 × 10⁻⁰⁸ | 2.8 × 10⁻⁰⁷ | 5 |
| | S27P, I28V, S50Y, | | | | | | |
| | N53P, G55W, S56D, | | | | | | |
| | T57W, N93R, A94W)- | | | | | | |
| | 35GS-ALB23002 | | | | | | |
| F010301555 | F0103275B05(E1D, | 1.4 × 10⁻⁰⁷ | 1.2 × 10⁻⁰⁶ | 8 | 9.2 × 10⁻⁰⁸ | 8.8 × 10⁻⁰⁷ | 10 |
| | S27P, 128V, S50Y, | | | | | | |
| | N53P, G55W, S56D, | | | | | | |
| | T57W, N93R, A94W)- | | | | | | |
| | 9GS-ALB23002 | | | | | | |

NA, not applicable;
ND, not determined

TABLE 45
Summary QPatch electrophysiology of ALB23002 HLE Nav1.7 ISVDs in presence/absence of human SA

| | | | HEK | | | HEK | |
|---|---|---|---|---|---|---|---|
| | | HEK | rhNav1.7-β1-β2-β3 + | Human | HEK | huNav1.7-β1 + | Human |
| | | rhNav1.7-β1-β2-β3 | 10 μM human | SA | huNav1.7-β1 | 10 μM human | SA |
| ID # | Description | IC50 [M] | SA IC50 [M] | ratio | IC50 [M] | SA IC50 [M] | ratio |
| F010301452 | F0103275B05(S27P, | 1.8 × 10⁻⁰⁸ | ND | NA | 2.1 × 10⁻⁰⁸ | ND | NA |
| | I28V, S50Y,N53P, | | | | | | |
| | G55W, S56D, T57W, | | | | | | |
| | N93R, A94W) | | | | | | |
| F010301465 | F0103275B05(E1D, | 5.7 × 10⁻⁰⁸ | 1.0 × 10⁻⁰⁷ | 2 | 4.3 × 10⁻⁰⁸ | 2.8 × 10₋₀₇ | 7 |
| | S27P, I28V, S50Y, | | | | | | |
| | N53P, G55W, S56D, | | | | | | |
| | T57W, N93R, A94W)- | | | | | | |
| | 35GS-ALB23002 | | | | | | |
| F010301555 | F0103275B05(E1D, | 3.5 × 10⁻⁰⁸ | 2.6 × 10⁻⁰⁷ | 7 | 4.7 × 10⁻⁰⁸ | 3.4 × 10⁻⁰⁷ | 7 |
| | S27P, I28V, S50Y, | | | | | | |
| | N53P, G55W, S56D, | | | | | | |
| | T57W, N93R, A94W)- | | | | | | |
| | 9GS-ALB23002 | | | | | | |

NA, not applicable;
ND, not determined

A number of huFc fusions were generated with the F0103265B04. The huFc moiety is based on hIgG1 with LALA and D265S mutations to reduce the interaction with FcγR. F0103265B04 is fused to the N-terminus of the huFc separated by a number of linkers with differing flexibilities as described elsewhere (Klein et al. Protein Eng Des Sel. 27:325-30 (2014), which is incorporated herein by reference in its entirety). Comparison of the different constructs in binding FACS revealed EC50 values comparable to monovalent F0103265B04 (Table 46), with the exception of 22ARO which suffered from a drop in potency. Interestingly, functional characterization using a single pulse electrophysiology protocol (FIG. 7A) revealed potencies highly favorable compared to monovalent F0103265B04 (last column of Table 46). Future experiments should determine whether these improvements are Fc- or linker-mediated.

TABLE 46
Summary binding FACS and Qpatch of F0103265B04-
Fc-fusions with different linkers

| | | FACS | | |
|---|---|---|---|---|
| | Description | HEK | FACS | |
| | [linker nomenclature | huNav1.7α- | HEK | Qpatch |
| | according to Klein et al. | β1-β2-β3 | huNav1.7α- | HEK |
| | 2014 Protein Eng Des Sel | cl.11 | β1 | huNav1.7α |
| ID # | 27:325] | EC50 [M] | EC50 [M] | IC50 [M] |
| F0103265B04 | F0103265B04-FLAG3- | ND | ND | 1.2 × 10⁻⁰⁷ |
| | HIS6 | | | |
| 22ARO | F0103265B04-L10 | 2.0 × 10⁻⁰⁸ | 2.9 × 10⁻⁰⁸ | 1.1 × 10⁻⁰⁷ |
| | GPZP-Fc | | | |
| 23ARO | F0103265B04-L1 hIgG- | 6.0 × 10⁻⁰⁹ | 8.8 × 10⁻⁰⁹ | 3.6 × 10⁻⁰⁸ |
| | Fc | | | |
| 24ARO | F0103265B04-L17 GS1- | 6.9 × 10⁻⁰⁹ | 9.2 × 10⁻⁰⁹ | 2.0 × 10⁻⁰⁸ |
| | Fc | | | |
| 25ARO | F0103265B04-L20 GS5- | 6.1 × 10⁻⁰⁹ | 8.2 × 10⁻⁰⁹ | 4.7 × 10⁻⁰⁸ |
| | Fc | | | |
| 26ARO | F0103265B04-L3 | 8.5 × 10⁻⁰⁹ | 1.4 × 10⁻⁰⁸ | 4.0 × 10⁻⁰⁹ |
| | GPGcP-Fc | | | |

ND, not determined

Another set of Nav1.7 binder-Fc fusion proteins was generated, this time with a 5GS linker separating the two moieties, and tested for binding and electrophysiology (Table 47) following the protocol depicted in FIG. 7A. Here, affinity maturation variants F010300659 (derived from F0103275B05) and F010301656 (derived from F0103387G04) were compared to parental F0103275B05. Addition of the Fc moiety does not appear to have a major impact on the functional potency.

TABLE 47
Summary binding FACS and QPatch characterization of ISVD-5GS-Fc-fusions

| | | | FACS | Qpatch | Qpatch |
|---|---|---|---|---|---|
| | | FACS | HEK | HEK | HEK |
| | | HEK | huNav1.7α- | rhNav1.7α- | huNav1.7α- |
| | | huNav1.7α | β1 | β1-β2-β3 | β1 |
| ID # | Description | EC50 [M] | EC50 [M] | IC50 [M] | IC50 [M] |
| F0103275B05 | F0103275B05- | 1.3 × 10⁻⁰⁸ | 1.3 × 10⁻⁰⁸ | ND | ND |
| | FLAG3 + HIS6 | | | | |
| 65ASP | F0103275B05- | 2.1 × 10⁻⁰⁸ | 4.5 × 10⁻⁰⁸ | ND | 1.2 × 10⁻⁰⁷ |
| | 5GS-Fc | | | | |
| F010300659 | F0103275B05(S27P, | 5.4 × 10⁻⁰⁹ | 1.1 × 10⁻⁰⁸ | 1.8 × 10⁻⁰⁷ | 8.5 × 10⁻⁰⁸ |
| | I28V, S50Y, N53P, | | | | |
| | G55W, S56D, T57W, | | | | |
| | N93R, A94W)- | | | | |
| | FLAG3 + HIS6 | | | | |
| 66ASP | F010300659-5GS- | 6.1 × 10⁻⁰⁸ | 4.2 × 10⁻⁰⁸ | 5.2 × 10⁻⁰⁸ | 9.2 × 10⁻⁰⁸ |
| | Fc | | | | |
| F010301656 | F0103387G04(K33R, | 4.9 × 10⁻⁰⁹ | 4.2 × 10⁻⁰⁹ | 6.0 × 10⁻⁰⁹ | 1.8 × 10⁻⁰⁸ |
| | S50Y, S56D, N93R)- | | | | |
| | FLAG3 + HIS6 | | | | |
| 69AVB | F010301656-5GS- | 1.1 × 10⁻⁰⁸ | 9.0 × 10⁻⁰⁹ | 8.0 × 10⁻⁰⁹ | 1.3 × 10⁻⁰⁸ |
| | Fc | | | | |

ND, not determined

In a last experiment, the potencies of different HLE versions of the F0103387G04 affinity maturation variant F010301656 were compared in competition FACS and electrophysiology on huNav1.7 and rhNav1.7 (Table 48 and Table 49). As described above, the addition of an ALB23002 or Fc moiety as HLE has no outspoken effect on the potency. The presence of a saturating concentration of human SA results in a ±5-fold drop in the potency of the ALB23002 fusion.

TABLE 48
Summary competition FACS of different HLE versions of ′1656 in presence/absence of human SA

| | | | CHO FlpIn | | | CHO FlpIn | |
|---|---|---|---|---|---|---|---|
| | | | huNav1.7α-β1- | | | rhNav1.7α-β1- | |
| | | CHO FlpIn | β2-β3 vs. EC25 | | CHO FlpIn | β2-β3 vs. EC25 | |
| | | huNav1.7α-β1- | of 275B05(N93R) | | rhNav1.7α-β1- | of 275B05(N93R) | |
| | | β2-β3 vs. EC25 | IC50 [M] + | Human | β2-β3 vs. EC25 | IC50 [M] + | Human |
| | | of 275B05(N93R) | 50 μM | SA | of 275B05(N93R) | 50 μM | SA |
| ID # | Description | IC50 [M] | human SA | ratio | IC50 [M] | human SA | ratio |
| F010301656 | F0103387G04(K33R, | 3.7 × 10⁻⁰⁸ | 3.9 × 10⁻⁰⁸ | 1 | 1.4 × 10⁻⁰⁸ | 1.6 × 10⁻⁰⁸ | 1 |
| | S50Y, S56D, N93R)- | | | | | | |
| | FLAG3-HIS6 | | | | | | |
| F010301940 | F0103387G04(E1D, | 3.5 × 10⁻⁰⁸ | 1.9 × 10⁻⁰⁷ | 5 | 1.3 × 10⁻⁰⁸ | 8.0 × 10⁻⁰⁸ | 6 |
| | K33R, S50Y, S56D, | | | | | | |
| | N93R)-35GS- | | | | | | |
| | ALB23002 | | | | | | |
| 69AVB | F010301656- | ND | ND | | ND | ND | |
| | 5GS-Fc | | | | | | |

ND, not determined

TABLE 49
Summary QPatch electrophysiology of different HLE versions of F010301656 in presence/absence of human SA

| | | | HEK | | | HEK | | |
|---|---|---|---|---|---|---|---|---|
| | | HEK | rhNav1.7α-β1-β2-β3 + | Human | HEK | huNav1.7α-β1 + | Human | HEK |
| | | rhNav1.7α-β1-β2-β3 | 10 μM human | SA | huNav1.7α-β1 | 10 □ M human | SA | ratNav1.7α |
| ID # | Description | IC50 [M] | SA IC50 [M] | ratio | IC50 [M] | SA IC50 [M] | ratio | IC50 [M] |
| F010301656 | F0103387G04 | 6.0 × 10⁻⁰⁹ | ND | ND | 1.8 × 10⁻⁰⁸ | ND | ND | ND |
| | (K33R, S50Y, | | | | | | | |
| | S56D, N93R)- | | | | | | | |
| | FLAG3-HIS6 | | | | | | | |
| F010301940 | F0103387G04 | 1.6 × 10⁻⁰⁸ | 5.0 × 10⁻⁰⁸ | 3 | 4.6 × 10⁻⁰⁸ | 1.5 × 10⁻⁰⁷ | 3 | 3.7 × 10⁻⁰⁸ |
| | (E1D, K33R, | | | | | | | |
| | S50Y, S56D, | | | | | | | |
| | N93R)-35GS- | | | | | | | |
| | ALB23002 | | | | | | | |
| 69AVB | F010301656- | 8.0 × 10⁻⁰⁹ | ND | ND | 1.3 × 10⁻⁰⁸ | 2.6 × 10⁻⁰⁸ | 2 | 2.1 × 10⁻⁰⁸ |
| | 5GS-Fc | | | | | | | |

ND, not determined

### Example 14

### Electrophysiological Characterization of Nav1.7a Selective ISVDs on the Automated Patch Clamp System QPatch.

Whole-cell currents were measured from cells stably expressing human, rhesus, or rat Nav1.7α, 1.6α, 1.5α, 1.4α channels using the QPatch HT™ (Sophion Bioscience). Cells were grown to 60-70% confluence in T175 cell culture flasks. Cells were lifted with Accutase™ and single cell suspensions generated with two million cells/mL.

Experiments were performed at room temperature (25-29° C.). Human and rhesus Nav1.7α currents were measured holding cells −85 mV and applying 30 ms test pulses to −20 mV at a frequency 0.1 Hz. Rat Nav1.7α currents were measured holding cells at −75 mV applying 30 ms test pulses to −20 mV at a frequency 0.1 Hz. Human and rhesus Nav1.6α, Nav1.5α, and Nav1.4α were held at −85 mV, −95 mV and −80 mV, respectively. The following solutions were used: Internal Solution (in mM): 30 CsCl, 5 HEPES, 10 EGTA, 120 CsF, 5 NaF, 2 MgCl₂, pH=7.3 with CsOH; External solutions (in mM) for human and rhesus Nav1.7α: 40 NaCl, 120 NMDG, 1 KCl, 0.5 MgCl₂, 5 HEPES, 2.7 CaCl₂, pH to 7.3 with NaOH; for rat Nav1.7α: 150 NaCl, 5 KCl, 2 CaCl₂, 1 MgCl₂, 10 HEPES, 12 Dextrose, pH 7.3 with NaOH. Sodium currents were monitored for at least five minutes in vehicle before addition of test articles. Double additions of test article were made to QPlate™ wells to achieve equilibrium. Current inhibition was measured after 60 pulses in test article. ProTX-II was used as positive control.

IC50 values, based on three concentrations, were calculated using a built-in four parameter logistic function (Hill equation): f(x)=Iₘᵢₙ+(Iₘₐₓ−Iₘᵢₙ)/(1±(IC50/[x])^{h}); Iₘᵢₙ=minimal current (fixed to 0); Iₘₐₓ=maximal current (fixed to a value of 100); IC50=half maximal inhibitory concentration; h=Hill coefficient.

Table 50, Table 51, Table 52, Table 53, Table 54, and Table 55 show the results. In Tables 50-55, N.E. means “no effect” and ND means “not determined”.

TABLE 50
Qpatch IC50s (nM) of Parental clones
Part 1

| | Human | | | | Rhesus |
|---|---|---|---|---|---|
| | Nav1.7α + | Human | Human | Human | Nav1.7α + |
| ID # | β1 | Nav1.6α | Nav1.5α | Nav1.4α | β1-β2-β3 |
| F0103265B04 | 120 | N.E. @14.5 μM | ND | ND | ND |
| F0103362B08 | 1 | ND | ND | ND | N.E. |
| F0103454D07 | 38 | ND | ND | ND | ND |
| F010346B09 | 7 | ND | ND | ND | 166 |

Part 2

| | Rhesus | Rhesus | Rhesus | Rat |
|---|---|---|---|---|
| ID # | Nav1.6α | Nav1.5α | Nav1.4α | Nav1.7α |
| F0103265B04 | ND | ND | ND | ND |
| F0103362B08 | ND | ND | ND | ND |
| F0103454D07 | ND | ND | ND | ND |
| F0103464B09 | ND | ND | ND | ND |

TABLE 51
Qpatch IC50s (nM) of F0103275B05 and F0103387G05 affinity-matured variants
Part 1

| | Human | | | | Rhesus |
|---|---|---|---|---|---|
| | Nav1.7α + | Human | Human | Human | Nav1.7α + |
| ID # | β1 | Nav1.6α | Nav1.5α | Nav1.4α | β1-β2-β3 |
| F10301656* | 13 | N.E. | N.E. | N.E. | 8 |
| | | @30 μM | @30 μM | @30 μM | |
| F010302383 | 22 | ND | ND | ND | 3 |
| F010300659 | 85 | N.E. | ND | ND | 199 |
| | | @6.3 μM | | | |
| F010300880 | 27 | ND | ND | ND | 122 |
| F010300900 | 196 | ND | ND | ND | 36 |
| F010300948 | 315 | ND | ND | ND | 58 |
| F010300990 | 249 | ND | ND | ND | 101 |
| F010300477 | 96 | ND | ND | ND | 1192 |
| F010300631 | 73 | ND | ND | ND | 173 |
| F010300684 | 202 | ND | ND | ND | 212 |

Part 2

| | Rhesus | Rhesus | Rhesus | Rat |
|---|---|---|---|---|
| ID # | Nav1.6α | Nav1.5α | Nav1.4α | Nav1.7α |
| F10301656* | N.E. @30 μM | N.E. @30 μM | N.E. @30 μM | 21 |
| F010302383 | N.E. @7 μM | N.E. @7 μM | N.E. @7 μM | ND |
| F010300659 | ND | ND | ND | ND |
| F010300880 | ND | ND | ND | ND |
| F010300900 | ND | ND | ND | ND |
| F010300948 | ND | ND | ND | ND |
| F010300990 | ND | ND | ND | ND |
| F010300477 | ND | ND | ND | ND |
| F010300631 | ND | ND | ND | ND |
| F010300684 | ND | ND | ND | ND |

*ISVD with human IgG1 Fc

TABLE 52
Qpatch IC50s (nM) of F0103265A11 affinity-matured variants
Part 1

| | Human | | | | Rhesus |
|---|---|---|---|---|---|
| | Nav1.7α + | Human | Human | Human | Nav1.7α + |
| ID # | β1 | Nav1.6α | Nav1.5α | Nav1.4α | β1-β2-β3 |
| F010301162 | 32 | ND | ND | ND | ND |
| F010301191 | 63 | ND | ND | ND | ND |
| F010301080 | 14 | ND | ND | ND | ND |
| F010301090 | 22 | ND | ND | ND | ND |
| F010301129 | 126 | ND | ND | ND | ND |

Part 2

| | Rhesus | Rhesus | Rhesus | Rat |
|---|---|---|---|---|
| ID # | Nav1.6α | Nav1.5α | Nav1.4α | Nav1.7α |
| F010301162 | ND | ND | ND | ND |
| F010301191 | ND | ND | ND | ND |
| F010301080 | ND | ND | ND | ND |
| F010301090 | ND | ND | ND | ND |
| F010301129 | ND | ND | ND | ND |

TABLE 53
Qpatch IC50s (nM) of F0103387G05 affinity-matured variants
Part 1

| | Human | | | | Rhesus |
|---|---|---|---|---|---|
| | Nav1.7α + | Human | Human | Human | Nav1.7α + |
| ID # | β1 | Nav1.6α | Nav1.5α | Nav1.4α | β1-β2-β3 |
| F010301558 | 8 | ND | ND | ND | ND |
| F010301559 | 12 | ND | ND | ND | ND |
| F010301563 | 5 | ND | ND | ND | ND |
| F010301566 | 18 | ND | ND | ND | ND |
| F010302391 | 32 | ND | ND | ND | ND |

Part 2

| | Rhesus | Rhesus | Rhesus | Rat |
|---|---|---|---|---|
| ID # | Nav1.6α | Nav1.5α | Nav1.4α | Nav1.7α |
| F010301558 | ND | ND | ND | ND |
| F010301559 | ND | ND | ND | ND |
| F010301563 | ND | ND | ND | ND |
| F010301566 | ND | ND | ND | ND |
| F010302391 | N.E. @7 μM | N.E. @7 μM | N.E. @7 μM | N.E. @7 μM |

TABLE 54
Qpatch IC50s (nM) of F0103464B09 affinity-matured variants
Part 1

| | Human | | | | Rhesus |
|---|---|---|---|---|---|
| | Nav1.7α + | Human | Human | Human | Nav1.7α + |
| ID # | β1 | Nav1.6α | Nav1.5α | Nav1.4α | β1-β2-β3 |
| F010302363 | 7 | ND | ND | ND | 166 |

Part 2

| | Rhesus | Rhesus | Rhesus | Rat |
|---|---|---|---|---|
| ID # | Nav1.6α | Nav1.5α | Nav1.4α | Nav1.7α |
| F010302363 | N.E. @7 μM | N.E. @7 μM | N.E. @7 μM | N.E. @7 μM |

TABLE 55
Qpatch IC50s (nM) of anti-Nav1.7α-Navβ bispecific ISVDs
Part 1

| | | | Human | Human |
|---|---|---|---|---|
| | | Human | Nav1.7α + | Nav1.2α + |
| ID # | Description | Nav1.7α | β1-β2-β3 | β1-β2 |
| F010300468 | F0103275B05(N93R) | 57 | 99 | ND |
| F010302375 | F0103275B05(E1D, N93R)-50GS- | 123 | 2.6 | N.E. |
| | F0103478E09(L108Q)-FLAG3- | | | |
| | HIS6 | | | |
| F010302378 | F0103275B05(E1D, N93R)-50GS- | 115 | 2.3 | N.E. |
| | F0103495F09-FLAG3-HIS6 | | | |
| F010302377 | F0103275B05(E1D, N93R)-50GS- | 74 | 0.8 | ND |
| | F0103492E09-FLAG3-HIS6 | | | |
| F010302379 | F0103275B05(E1D, N93R)-50GS- | 111 | 3.4 | ND |
| | F0103500E03(P14A, L108Q)- | | | |
| | FLAG3-HIS6 | | | |

Part 2

| | | | Rhesus |
|---|---|---|---|
| | | Rhesus | Nav1.7α + |
| ID # | Description | Nav1.7α | β1-β2-β3 |
| F010300468 | F0103275B05(N93R) | ND | ND |
| F010302375 | F0103275B05(E1D, N93R)-50GS- | 93 | 2.8 |
| | F0103478E09(L108Q)-FLAG3- | | |
| | HIS6 | | |
| F010302378 | F0103275B05(E1D, N93R)-50GS- | 103 | 2.6 |
| | F0103495F09-FLAG3-HIS6 | | |
| F010302377 | F0103275B05(E1D, N93R)-50GS- | 104 | 75 |
| | F0103492E09-FLAG3-HIS6 | | |
| F010302379 | F0103275B05(E1D, N93R)-50GS- | 133 | 131 |
| | F0103500E03(P14A, L108Q)- | | |
| | FLAG3-HIS6 | | |

The amino acid and nucleotide sequences for the Nav1.7 binders, CDRs, and other molecules disclosed herein are set forth Table 56.

TABLE 56
Table of Sequences

| SEQ | | |
|---|---|---|
| ID | | |
| NO: | Description | Sequence |
| 1 | huNav1.7 (alpha- | MAMLPPPGPQSFVHFTKQSLALIEQRIAERKSKEPK |
| | subunit) | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKK |
| 2 | rhNav1.7 (alpha- | MAMLPPPGPQSFVHFTKQSLALIEQRIAERKSKEPK |
| | subunit) | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKK |
| 3 | huNav1.7-beta1-beta2- | MAMLPPPGPQSFVHFTKQSLALIEQRIAERKSKEPK |
| | beta3 viral P2A | EEKKDDDEEAPKPSSDLEAGKQLPFIYGDIPPGMVS |
| | sequences italics; beta1- | EPLEDLDPYYADKKTFIVLNKGKTIFRFNATPALYM |
| | beta2-beta3 are in bold | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKSGRGSGATNFSLLKQAGDVEENPGPMGRLLA |
| | | LVVGAALVSSACGGCVEVDSETEAVYGMTFKIL |
| | | CISCKRRSETNAETFTEWTFRQKGTEEFVKILRY |
| | | ENEVLQLEEDERFEGRVVWNGSRGTKDLQDLSI |
| | | FITNVTYNHSGDYECHVYRLLFFENYEHNTSVV |
| | | KKIHIEVVDKANRDMASIVSEIMMYVLIVVLTIW |
| | | LVAEMIYCYKKIAAATETAAQENASEYLAITSES |
| | | KENCTGVQVAEGSGATNFSLLKQAGDVEENPGPM |
| | | HRDAWLPRPAFSLTGLSLFFSLVPPGRSMEVTVP |
| | | ATLNVLNGSDARLPCTFNSCYTVNHKQFSLNWT |
| | | YQECNNCSEEMFLQFRMKIINLKLERFQDRVEF |
| | | SGNPSKYDVSVMLRNVQPEDEGIYNCYIMNPPD |
| | | RHRGHGKIHLQVLMEEPPERDSTVAVIVGASVG |
| | | GFLAVVILVLMVVKCVRRKKEQKLSTDDLKTEE |
| | | EGKTDGEGNPDDGAKGSGATNFSLLKQAGDVEEN |
| | | PGPMPAFNRLFPLASLVLIYWVSVCFPVCVEVPS |
| | | ETEAVQGNPMKLRCISCMKREEVEATTVVEWF |
| | | YRPEGGKDFLIYEYRNGHQEVESPFQGRLQWN |
| | | GSKDLQDVSITVLNVTLNDSGLYTCNVSREFEFE |
| | | AHRPFVKTTRLIPLRVTEEAGEDFTSVVSEIMMY |
| | | ILLVFLTLWLLIEMIYCYRKVSKAEEAAQENASD |
| | | YLAIPSENKENSAVPVEE |
| 4 | rhNav1.7-beta1-beta2- | MAMLPPPGPQSFVHFTKQSLALIEQRIAERKSKEPK |
| | beta3 viral P2A | EEKKDDDEEAPKPSSDLEAGKQLPFIYGDIPPGMVS |
| | sequences italics; beta1- | EPLEDLDPYYADKKTFIVLNKGKTIFRFNATPALYM |
| | beta2-beta3 are in bold | LSPFSPLRRISIKILVHSLFSMLIMCTILTNCIFMTMS |
| | | NPPDWTKNVEYTFTGIYTFESLVKILARGFCVGEFT |
| | | FLRDPWNWLDFIVIVFAYLTEFVNLGNVSALRTFR |
| | | VLRALKTISVIPGLKTIVGALIQSVKKLSDVMILTVF |
| | | CLSVFALIGLQLFMGNLKHKCVQNSLVNNETLESI |
| | | MNTLESEEDFRKYFYYLEGSKDALLCGFSTDSGQC |
| | | PEGYTCMKIGRNPDYGYTSFDTFSWAFLALFRLMT |
| | | QDYWENLYQQTLRAAGKTYMIFFVVVIFLGSFYLI |
| | | NLILAVVAMAYEEQNQANIEEAKQKELEFQQMLD |
| | | RLKKEQEEAEAIAAAAAEYTSIRRSRIMGLSESSSET |
| | | SKLSSKSAKERRNRRKKKNQKKLSSGEEKGDAEKL |
| | | SKSDSEENIRRKSFHLGVEGHRRAHEKRLSTPSQSP |
| | | LSIRGSLFSARRSSRTSLFSFKGRGRDIGSETEFADD |
| | | EHSIFGDNESRRGSLFVPHRPQERRSSNISQASRSPPI |
| | | LPVNGKMHSAVDCNGVVSLVDGRSALMLPNGQLL |
| | | PEGTTNQIHKKRRCSSYLLSEDMLNDPNLRQRAMS |
| | | RASILTNTVEELEESRQKCPPWWYRFAHKFLIWNC |
| | | SPYWIKFKKCIYFIVMDPFVDLAITICIVLNTLFMAM |
| | | EHHPMTEEFKNVLAIGNLVFTGIFAAEMVLKLIAM |
| | | DPYEYFQVGWNIFDSLIVTLSLVELFLADVEGLSVL |
| | | RSFRLLRVFKLAKSWPTLNMLIKIIGNSVGALGNLT |
| | | LVLAIIVFIFAVVGMQLFGKSYKECVCKINDDCTLP |
| | | RWHMNDFFHSFLIVFRVLCGEWIETMWDCMEVAG |
| | | QAMCLIVYMMVMVIGNLVVLNLFLALLLSSFSSDN |
| | | LTAIEEDPDANNLQIAVTRIKKGINYVKQTLREFILK |
| | | TFSKKPKISREIRQTEDLNTKKENYISNYTLAEMSK |
| | | GHNFLKEKDKISGFGSCVDKYLMEDSDGQSFIHNP |
| | | SLTVTVPIAPGESDLENMNTEELSSDSDSEYSKVRL |
| | | NQSSSSECSTVDNPLPGEGEEAEAEPMNSDEPEACF |
| | | TDGCVRRFSCCQVNIESGKGKIWWNIRKTCYKIVE |
| | | HSWFESFIVLMILLSSGALAFEDIYIERKKTIKIILEY |
| | | ADKIFTYIFILEMLLKWIAYGYKTYFTNAWCWLDF |
| | | LIVDVSLVTLVANTLGYSDLGPIKSLRTLRALRPLR |
| | | ALSRFEGMRVVVNALIGAIPSIMNVLLVCLIFWLIFS |
| | | IMGVNLFAGKFYECINTTDGSRFPASQVPNRSECFA |
| | | LMNVSQNVRWKNLKVNFDNVGLGYLSLLQVATF |
| | | KGWTIIMYAAVDSVNVDKQPKYEYSLYMYIYFVIF |
| | | IIFGSFFTLNLFIGVIIDNFNQQKKKLGGQDIFMTEEQ |
| | | KKYYNAMKKLGSKKPQKPIPRPGNKIQGCIFDLVT |
| | | NQAFDISIMVLICLNMVTMMVEKEGQSPYMTDVL |
| | | YWINVVFIILFTGECVLKLISLRYYYFTIGWNIFDFV |
| | | VVIISIVGMFLADLIETYFVSPTLFRVIRLARIGRILRL |
| | | VKGAKGIRTLLFALMMSLPALFNIGLLLFLVMFIYA |
| | | IFGMSNFAYVKKEDGINDMFNFETFGNSMICLFQIT |
| | | TSAGWDGLLAPILNSKPPDCDPKKVHPGSSVEGDC |
| | | GNPSVGIFYFVSYIIISFLVVVNMYIAVILENFSVATE |
| | | ESTEPLSEDDFEMFYEVWEKFDPDATQFIEYNKLSD |
| | | FAAALDPPLLIAKPNKVQLIAMDLPMVSGDRIHCL |
| | | DILFAFTKRVLGESGEMDSLRSQMEERFMSANPSK |
| | | VSYEPITTTLKRKQEDVSATVIQRAYRRYRLRQNV |
| | | KNISSIYIKDGDRDDDLLNKKDMAFDNVNENSSPE |
| | | KTDATSSTTSPPSYDSVTKPDKEKYEQDRTEKEDK |
| | | GKDSKESKKSGRGSGATNFSLLKQAGDVEENPGPM |
| | | GRLLALVVGAALVSSACGGCVEVDSETEAVYG |
| | | MTFKILCISCKRRSETNAETFTEWTFRQKGTEEF |
| | | VKILRYENEVLQLEEDERFEGRVVWNGSRGTKD |
| | | LQDLSIFITNVTYNHSGDYECHVYRLLFFENYEH |
| | | NTSVVKKIHIEVVDKANRDMASIVSEIMMYVLIV |
| | | VLTIWLVAEMIYCYKKIAAATETAAQENASEYL |
| | | AITSESKENCTGVQVAEGSGATNFSLLKQAGDVEE |
| | | NPGPMHRDAWLPRPAFSLTGLSLFFSLVPPGRS |
| | | MEVTVPATLNVLNGSDARLPCTFNSCYTVNHKQ |
| | | FSLNWTYQECNNCSEEMFLQFRMKIINLKLERF |
| | | QDRVEFSGNPSKYDVSVMLRNVQPEDEGIYNCYI |
| | | MNPPDRHRGHGKIHLQVLMEEPPERDSTVAVIV |
| | | GASVGGFLAVVILVLMVVKCVRRKKEQKLSTD |
| | | DLKTEEEGKTDGEGNPDDGAKGSGATNFSLLKQA |
| | | GDVEENPGPMPAFNRLFPLASLVLIYWVSVCFPV |
| | | CVEVPSETEAVQGNPMKLRCISCMKREEVEATT |
| | | VVEWFYRPEGGKDFLIYEYRNGHQEVESPFQGR |
| | | LQWNGSKDLQDVSITVLNVTLNDSGLYTCNVSR |
| | | EFEFEAHRPFVKTTRLIPLRVTEEAGEDFTSVVS |
| | | EIMMYILLVFLTLWLLIEMIYCYRKVSKAEEAA |
| | | QENASDYLAIPSENKENSAVPVEE |
| 5 | huNav1.7(F276V)- | MAMLPPPGPQSFVHFTKQSLALIEQRIAERKSKEPK |
| | beta1-beta2-beta3 viral | EEKKDDDEEAPKPSSDLEAGKQLPFIYGDIPPGMVS |
| | P2A sequences italics; | EPLEDLDPYYADKKTFIVLNKGKTIFRFNATPALYM |
| | beta1-beta2-beta3 are in | LSPFSPLRRISIKILVHSLFSMLIMCTILTNCIFMTMN |
| | bold | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCVRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKENLYFQGDYKDHDGDYKDHDIDYKDDDDK |
| | | HHHHHHHHHHGSGATNFSLLKQAGDVEENPGPMG |
| | | RLLALVVGAALVSSACGGCVEVDSETEAVYGMT |
| | | FKILCISCKRRSETNAETFTEWTFRQKGTEEFVK |
| | | ILRYENEVLQLEEDERFEGRVVWNGSRGTKDLQ |
| | | DLSIFITNVTYNHSGDYECHVYRLLFFENYEHNT |
| | | SVVKKIHIEVVDKANRDMASIVSEIMMYVLIVVL |
| | | TIWLVAEMIYCYKKIAAATETAAQENASEYLAIT |
| | | SESKENCTGVQVAEGSGATNFSLLKQAGDVEENPG |
| | | PMHRDAWLPRPAFSLTGLSLFFSLVPPGRSMEV |
| | | TVPATLNVLNGSDARLPCTFNSCYTVNHKQFSL |
| | | NWTYQECNNCSEEMFLQFRMKIINLKLERFQDR |
| | | VEFSGNPSKYDVSVMLRNVQPEDEGIYNCYIMN |
| | | PPDRHRGHGKIHLQVLMEEPPERDSTVAVIVGA |
| | | SVGGFLAVVILVLMVVKCVRRKKEQKLSTDDLK |
| | | TEEEGKTDGEGNPDDGAKGSGATNFSLLKQAGDV |
| | | EENPGPMPAFNRLFPLASLVLIYWVSVCFPVCVE |
| | | VPSETEAVQGNPMKLRCISCMKREEVEATTVVE |
| | | WFYRPEGGKDFLIYEYRNGHQEVESPFQGRLQ |
| | | WNGSKDLQDVSITVLNVTLNDSGLYTCNVSREF |
| | | EFEAHRPFVKTTRLIPLRVTEEAGEDFTSVVSEI |
| | | MMYILLVFLTLWLLIEMIYCYRKVSKAEEAAQE |
| | | NASDYLAIPSENKENSAVPVEE |
| 6 | huNav1.7(R277Q)- | MAMLPPPGPQSFVHFTKQSLALIEQRIAERKSKEPK |
| | beta1-beta2-beta3 viral | EEKKDDDEEAPKPSSDLEAGKQLPFIYGDIPPGMVS |
| | P2A sequences italics; | EPLEDLDPYYADKKTFIVLNKGKTIFRFNATPALYM |
| | beta1-beta2-beta3 are in | LSPFSPLRRISIKILVHSLFSMLIMCTILTNCIFMTMN |
| | bold | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFQNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKENLYFQGDYKDHDGDYKDHDIDYKDDDDK |
| | | HHHHHHHHHHGSGATNFSLLKQAGDVEENPGPMG |
| | | RLLALVVGAALVSSACGGCVEVDSETEAVYGMT |
| | | FKILCISCKRRSETNAETFTEWTFRQKGTEEFVK |
| | | ILRYENEVLQLEEDERFEGRVVWNGSRGTKDLQ |
| | | DLSIFITNVTYNHSGDYECHVYRLLFFENYEHNT |
| | | SVVKKIHIEVVDKANRDMASIVSEIMMYVLIVVL |
| | | TIWLVAEMIYCYKKIAAATETAAQENASEYLAIT |
| | | SESKENCTGVQVAEGSGATNFSLLKQAGDVEENPG |
| | | PMHRDAWLPRPAFSLTGLSLFFSLVPPGRSMEV |
| | | TVPATLNVLNGSDARLPCTFNSCYTVNHKQFSL |
| | | NWTYQECNNCSEEMFLQFRMKIINLKLERFQDR |
| | | VEFSGNPSKYDVSVMLRNVQPEDEGIYNCYIMN |
| | | PPDRHRGHGKIHLQVLMEEPPERDSTVAVIVGA |
| | | SVGGFLAVVILVLMVVKCVRRKKEQKLSTDDLK |
| | | TEEEGKTDGEGNPDDGAKGSGATNFSLLKQAGDV |
| | | EENPGPMPAFNRLFPLASLVLIYWVSVCFPVCVE |
| | | VPSETEAVQGNPMKLRCISCMKREEVEATTVVE |
| | | WFYRPEGGKDFLIYEYRNGHQEVESPFQGRLQ |
| | | WNGSKDLQDVSITVLNVTLNDSGLYTCNVSREF |
| | | EFEAHRPFVKTTRLIPLRVTEEAGEDFTSVVSEI |
| | | MMYILLVFLTLWLLIEMIYCYRKVSKAEEAAQE |
| | | NASDYLAIPSENKENSAVPVEE |
| 7 | huNav1.7(E281V)- | MAMLPPPGPQSFVHFTKQSLALIEQRIAERKSKEPK |
| | beta1-beta2-beta3 | EEKKDDDEEAPKPSSDLEAGKQLPFIYGDIPPGMVS |
| | viral P2A sequences | EPLEDLDPYYADKKTFIVLNKGKTIFRFNATPALYM |
| | italics; beta1-beta2- | LSPFSPLRRISIKILVHSLFSMLIMCTILTNCIFMTMN |
| | beta3 are in bold | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLVNNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKENLYFQGDYKDHDGDYKDHDIDYKDDDDK |
| | | HHHHHHHHHHGSGATNFSLLKQAGDVEENPGPMG |
| | | RLLALVVGAALVSSACGGCVEVDSETEAVYGMTF |
| | | KILCISCKRRSETNAETFTEWTFRQKGTEEFVKILRY |
| | | ENEVLQLEEDERFEGRVVWNGSRGTKDLQDLSIFIT |
| | | NVTYNHSGDYECHVYRLLFFENYEHNTSVVKKIHI |
| | | EVVDKANRDMASIVSEIMMYVLIVVLTIWLVAEMI |
| | | YCYKKIAAATETAAQENASEYLAITSESKENCTGV |
| | | QVAEGSGATNFSLLKQAGDVEENPGPMHRDAWLP |
| | | RPAFSLTGLSLFFSLVPPGRSMEVTVPATLNVLN |
| | | GSDARLPCTFNSCYTVNHKQFSLNWTYQECNNC |
| | | SEEMFLQFRMKIINLKLERFQDRVEFSGNPSKYD |
| | | VSVMLRNVQPEDEGIYNCYIMNPPDRHRGHGKI |
| | | HLQVLMEEPPERDSTVAVIVGASVGGFLAVVILV |
| | | LMVVKCVRRKKEQKLSTDDLKTEEEGKTDGEG |
| | | NPDDGAKGSGATNFSLLKQAGDVEENPGPMPAFNR |
| | | LFPLASLVLIYWVSVCFPVCVEVPSETEAVQGNP |
| | | MKLRCISCMKREEVEATTVVEWFYRPEGGKDF |
| | | LIYEYRNGHQEVESPFQGRLQWNGSKDLQDVSI |
| | | TVLNVTLNDSGLYTCNVSREFEFEAHRPFVKTTR |
| | | LIPLRVTEEAGEDFTSVVSEIMMYILLVFLTLWL |
| | | LIEMIYCYRKVSKAEEAAQENASDYLAIPSENKE |
| | | NSAVPVEE |
| 8 | huNav1.7(V331M)- | MAMLPPPGPQSFVHFTKQSLALIEQRIAERKSKEPK |
| | beta1-beta2-beta3 viral | EEKKDDDEEAPKPSSDLEAGKQLPFIYGDIPPGMVS |
| | P2A sequences italics; | EPLEDLDPYYADKKTFIVLNKGKTIFRFNATPALYM |
| | beta1-beta2-beta3 are in | LSPFSPLRRISIKILVHSLFSMLIMCTILTNCIFMTMN |
| | bold | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCMKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKENLYFQGDYKDHDGDYKDHDIDYKDDDDK |
| | | HHHHHHHHHHGSGATNFSLLKQAGDVEENPGPMG |
| | | RLLALVVGAALVSSACGGCVEVDSETEAVYGMT |
| | | FKILCISCKRRSETNAETFTEWTFRQKGTEEFVK |
| | | ILRYENEVLQLEEDERFEGRVVWNGSRGTKDLQ |
| | | DLSIFITNVTYNHSGDYECHVYRLLFFENYEHNT |
| | | SVVKKIHIEVVDKANRDMASIVSEIMMYVLIVVL |
| | | TIWLVAEMIYCYKKIAAATETAAQENASEYLAIT |
| | | SESKENCTGVQVAEGSGATNFSLLKQAGDVEENPG |
| | | PMHRDAWLPRPAFSLTGLSLFFSLVPPGRSMEV |
| | | TVPATLNVLNGSDARLPCTFNSCYTVNHKQFSL |
| | | NWTYQECNNCSEEMFLQFRMKIINLKLERFQDR |
| | | VEFSGNPSKYDVSVMLRNVQPEDEGIYNCYIMN |
| | | PPDRHRGHGKIHLQVLMEEPPERDSTVAVIVGA |
| | | SVGGFLAVVILVLMVVKCVRRKKEQKLSTDDLK |
| | | TEEEGKTDGEGNPDDGAKGSGATNFSLLKQAGDV |
| | | EENPGPMPAFNRLFPLASLVLIYWVSVCFPVCVE |
| | | VPSETEAVQGNPMKLRCISCMKREEVEATTVVE |
| | | WFYRPEGGKDFLIYEYRNGHQEVESPFQGRLQ |
| | | WNGSKDLQDVSITVLNVTLNDSGLYTCNVSREF |
| | | EFEAHRPFVKTTRLIPLRVTEEAGEDFTSVVSEI |
| | | MMYILLVFLTLWLLIEMIYCYRKVSKAEEAAQE |
| | | NASDYLAIPSENKENSAVPVEE |
| 9 | huNav1.7(N146S, V194I, | MAMLPPPGPQSFVHFTKQSLALIEQRIAERKSKEPK |
| | F276V, R277Q, E281V, | EEKKDDDEEAPKPSSDLEAGKQLPFIYGDIPPGMVS |
| | V331M, E504D, D507E, | EPLEDLDPYYADKKTFIVLNKGKTIFRFNATPALYM |
| | S508N, N533S)-beta1- | LSPFSPLRRISIKILVHSLFSMLIMCTILTNCIFMTMS |
| | beta2-beta3 viral P2A | NPPDWTKNVEYTFTGIYTFESLVKILARGFCVGEFT |
| | sequences italics; beta1- | FLRDPWNWLDFIVIVFAYLTEFVNLGNVSALRTFR |
| | beta2-beta3 are in bold | VLRALKTISVIPGLKTIVGALIQSVKKLSDVMILTVF |
| | | CLSVFALIGLQLFMGNLKHKCVQNSLVNNETLESI |
| | | MNTLESEEDFRKYFYYLEGSKDALLCGFSTDSGQC |
| | | PEGYTCMKIGRNPDYGYTSFDTFSWAFLALFRLMT |
| | | QDYWENLYQQTLRAAGKTYMIFFVVVIFLGSFYLI |
| | | NLILAVVAMAYEEQNQANIEEAKQKELEFQQMLD |
| | | RLKKEQEEAEAIAAAAAEYTSIRRSRIMGLSESSSET |
| | | SKLSSKSAKERRNRRKKKNQKKLSSGEEKGDAEKL |
| | | SKSDSEENIRRKSFHLGVEGHRRAHEKRLSTPSQSP |
| | | LSIRGSLFSARRSSRTSLFSFKGRGRDIGSETEFADD |
| | | EHSIFGDNESRRGSLFVPHRPQERRSSNISQASRSPP |
| | | MLPVNGKMHSAVDCNGVVSLVDGRSALMLPNGQ |
| | | LLPEGTTNQIHKKRRCSSYLLSEDMLNDPNLRQRA |
| | | MSRASILTNTVEELEESRQKCPPWWYRFAHKFLIW |
| | | NCSPYWIKFKKCIYFIVMDPFVDLAITICIVLNTLFM |
| | | AMEHHPMTEEFKNVLAIGNLVFTGIFAAEMVLKLI |
| | | AMDPYEYFQVGWNIFDSLIVTLSLVELFLADVEGLS |
| | | VLRSFRLLRVFKLAKSWPTLNMLIKIIGNSVGALGN |
| | | LTLVLAIIVFIFAVVGMQLFGKSYKECVCKINDDCT |
| | | LPRWHMNDFFHSFLIVFRVLCGEWIETMWDCMEV |
| | | AGQAMCLIVYMMVMVIGNLVVLNLFLALLLSSFSS |
| | | DNLTAIEEDPDANNLQIAVTRIKKGINYVKQTLREFI |
| | | LKAFSKKPKISREIRQAEDLNTKKENYISNHTLAEM |
| | | SKGHNFLKEKDKISGFGSSVDKHLMEDSDGQSFIH |
| | | NPSLTVTVPIAPGESDLENMNAEELSSDSDSEYSKV |
| | | RLNRSSSSECSTVDNPLPGEGEEAEAEPMNSDEPEA |
| | | CFTDGCVRRFSCCQVNIESGKGKIWWNIRKTCYKI |
| | | VEHSWFESFIVLMILLSSGALAFEDIYIERKKTIKIIL |
| | | EYADKIFTYIFILEMLLKWIAYGYKTYFTNAWCWL |
| | | DFLIVDVSLVTLVANTLGYSDLGPIKSLRTLRALRP |
| | | LRALSRFEGMRVVVNALIGAIPSIMNVLLVCLIFWL |
| | | IFSIMGVNLFAGKFYECINTTDGSRFPASQVPNRSEC |
| | | FALMNVSQNVRWKNLKVNFDNVGLGYLSLLQVA |
| | | TFKGWTIIMYAAVDSVNVDKQPKYEYSLYMYIYF |
| | | VVFIIFGSFFTLNLFIGVIIDNFNQQKKKLGGQDIFM |
| | | TEEQKKYYNAMKKLGSKKPQKPIPRPGNKIQGCIF |
| | | DLVTNQAFDISIMVLICLNMVTMMVEKEGQSQHM |
| | | TEVLYWINVVFIILFTGECVLKLISLRHYYFTVGWNI |
| | | FDFVVVIISIVGMFLADLIETYFVSPTLFRVIRLARIG |
| | | RILRLVKGAKGIRTLLFALMMSLPALFNIGLLLFLV |
| | | MFIYAIFGMSNFAYVKKEDGINDMFNFETFGNSMI |
| | | CLFQITTSAGWDGLLAPILNSKPPDCDPKKVHPGSS |
| | | VEGDCGNPSVGIFYFVSYIIISFLVVVNMYIAVILEN |
| | | FSVATEESTEPLSEDDFEMFYEVWEKFDPDATQFIE |
| | | FSKLSDFAAALDPPLLIAKPNKVQLIAMDLPMVSG |
| | | DRIHCLDILFAFTKRVLGESGEMDSLRSQMEERFMS |
| | | ANPSKVSYEPITTTLKRKQEDVSATVIQRAYRRYRL |
| | | RQNVKNISSIYIKDGDRDDDLLNKKDMAFDNVNEN |
| | | SSPEKTDATSSTTSPPSYDSVTKPDKEKYEQDRTEK |
| | | EDKGKDSKESKKSGRGSGATNFSLLKQAGDVEENPG |
| | | PMGRLLALVVGAALVSSACGGCVEVDSETEAVY |
| | | GMTFKILCISCKRRSETNAETFTEWTFRQKGTE |
| | | EFVKILRYENEVLQLEEDERFEGRVVWNGSRGT |
| | | KDLQDLSIFITNVTYNHSGDYECHVYRLLFFENY |
| | | EHNTSVVKKIHIEVVDKANRDMASIVSEIMMYV |
| | | LIVVLTIWLVAEMIYCYKKIAAATETAAQENASE |
| | | YLAITSESKENCTGVQVAEGSGATNFSLLKQAGDV |
| | | EENPGPMHRDAWLPRPAFSLTGLSLFFSLVPPGR |
| | | SMEVTVPATLNVLNGSDARLPCTFNSCYTVNHK |
| | | QFSLNWTYQECNNCSEEMFLQFRMKIINLKLER |
| | | FQDRVEFSGNPSKYDVSVMLRNVQPEDEGIYNC |
| | | YIMNPPDRHRGHGKIHLQVLMEEPPERDSTVAV |
| | | IVGASVGGFLAVVILVLMVVKCVRRKKEQKLST |
| | | DDLKTEEEGKTDGEGNPDDGAKGSGATNFSLLK |
| | | QAGDVEENPGPMPAFNRLFPLASLVLIYWVSVCF |
| | | PVCVEVPSETEAVQGNPMKLRCISCMKREEVEA |
| | | TTVVEWFYRPEGGKDFLIYEYRNGHQEVESPFQ |
| | | GRLQWNGSKDLQDVSITVLNVTLNDSGLYTCNV |
| | | SREFEFEAHRPFVKTTRLIPLRVTEEAGEDFTSV |
| | | VSEIMMYILLVFLTLWLLIEMIYCYRKVSKAEEA |
| | | AQENASDYLAIPSENKENSAVPVEE |
| 10 | huNav157 chimera 1 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPLNKYQGFIFDIVTKQ |
| | | AFDVTIMFLICLNMVTMMVETDDQSPEKINILAKIN |
| | | LLFVAIFTGECIVKLAALRHYYFTNSWNIFDFVVVI |
| | | LSIVGTVLSDIIQKYFFSPTLFRVIRLARIGRILRLIRG |
| | | AKGIRTLLFALMMSLPALFNIGLLLFLVMFIYSIFGM |
| | | ANFAYVKWEAGIDDMFNFQTFANSMLCLFQITTSA |
| | | GWDGLLSPILNTGPPYCDPTLPNSNGSRGDCGSPAV |
| | | GILFFTTYIIISFLIVVNMYIAIILENFSVATEESTEPLS |
| | | EDDFDMFYEIWEKFDPEATQFIEYSVLSDFADALSE |
| | | PLRIAKPNQISLINMDLPMVSGDRIHCMDILFAFTKR |
| | | VLGESGEMDALKIQMEEKFMAANPSKISYEPITTTL |
| | | RRKHEEVSAMVIQRAFRRHLLQRSLKHASFLFRQQ |
| | | AGSGLSEEDAPEREGLIAYVMSENFSRPLGPPSSSSI |
| | | SSTSFPPSYDSVTRATSDNLQVRGSDYSHSEDLADF |
| | | PPSPDRDRESIV |
| 11 | huNav157 chimera 2 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSADNLT |
| | | APDEDREMNNLQLALARIQRGLRFVKRTTWDFCC |
| | | GLLRQRPQKPAALAAQGQLPSCIATPYSPPPPETEK |
| | | VPPTRKETRFEEGEQPGQGTPGDPEPVCVPIAVAES |
| | | DTDDQEEDEENSLGTEEESSKQESQPVSGGPEAPPD |
| | | SRTWSQVSATASSEAEASASQADWRQQWKAEPQA |
| | | PGCGETPEDSCSEGSTADMINTAELLEQIPDLGQDV |
| | | KDPEDCFTEGCVRRCPCCAVDTTQAPGKVWWRLR |
| | | KTCYHIVEHSWFETFIIFMILLSSGALAFEDIYLEER |
| | | KTIKVLLEYADKMFTYVFVLEMLLKWVAYGFKKY |
| | | FTNAWCWLDFLIVDVSLVSLVANTLGFAEMGPIKS |
| | | LRTLRALRPLRALSRFEGMRVVVNALVGAIPSIMN |
| | | VLLVCLIFWLIFSIMGVNLFAGKFGRCINQTEGDLP |
| | | LNYTIVNNKSQCESLNLTGELYWTKVKVNFDNVG |
| | | AGYLALLQVATFKGWMDIMYAAVDSRGYEEQPQ |
| | | WEYNLYMYIYFVIFIIFGSFFTLNLFIGVIIDNFNQQK |
| | | KKLGGQDIFMTEEQKKYYNAMKKLGSKKPQKPIP |
| | | RPGNKIQGCIFDLVTNQAFDISIMVLICLNMVTMMV |
| | | EKEGQSQHMTEVLYWINVVFIILFTGECVLKLISLR |
| | | HYYFTVGWNIFDFVVVIISIVGMFLADLIETYFVSPT |
| | | LFRVIRLARIGRILRLVKGAKGIRTLLFALMMSLPA |
| | | LFNIGLLLFLVMFIYAIFGMSNFAYVKKEDGINDMF |
| | | NFETFGNSMICLFQITTSAGWDGLLAPILNSKPPDC |
| | | DPKKVHPGSSVEGDCGNPSVGIFYFVSYIIISFLVVV |
| | | NMYIAVILENFSVATEESTEPLSEDDFEMFYEVWEK |
| | | FDPDATQFIEFSKLSDFAAALDPPLLIAKPNKVQLIA |
| | | MDLPMVSGDRIHCLDILFAFTKRVLGESGEMDSLR |
| | | SQMEERFMSANPSKVSYEPITTTLKRKQEDVSATVI |
| | | QRAYRRYRLRQNVKNISSIYIKDGDRDDDLLNKKD |
| | | MAFDNVNENSSPEKTDATSSTTSPPSYDSVTKPDKE |
| | | KYEQDRTEKEDKGKDSKESKK |
| 12 | huNav157 chimera 3 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQATIAETEEKEKRFQEAMEMLK |
| | | KEHEALTIRGVDTVSRSSLEMSPLAPVNSHERRSKR |
| | | RKRMSSGTEECGEDRLPKSDSEDGPRAMNHLSLTR |
| | | GLSRTSMKPRSSRGSIFTFRRRDLGSEADFADDENS |
| | | TAGESESHHTSLLVPWPLRRTSAQGQPSPGTSAPGH |
| | | ALHGKKNSTVDCNGVVSLLGAGDPEATSPGSHLLR |
| | | PVMLEHPPDTTTPSEEPGGPQMLTSQAPCVDGFEEP |
| | | GARQRALSAVSVLTSALEELEESRHKCPPCWNRLA |
| | | QRYLIWECCPLWMSIKQGVKLVVMDPFTDLTITMC |
| | | IVLNTLFMALEHYNMTSEFEEMLQVGNLVFTGIFT |
| | | AEMTFKIIALDPYYYFQQGWNIFDSIIVILSLMELGL |
| | | SRMSNLSVLRSFRLLRVFKLAKSWPTLNTLIKIIGNS |
| | | VGALGNLTLVLAIIVFIFAVVGMQLFGKNYSELRDS |
| | | DSGLLPRWHMMDFFHAFLIIFRILCGEWIETMWDC |
| | | MEVSGQSLCLLVFLLVMVIGNLVVLNLFLALLLSSF |
| | | SSDNLTAIEEDPDANNLQIAVTRIKKGINYVKQTLR |
| | | EFILKAFSKKPKISREIRQAEDLNTKKENYISNHTLA |
| | | EMSKGHNFLKEKDKISGFGSSVDKHLMEDSDGQSF |
| | | IHNPSLTVTVPIAPGESDLENMNAEELSSDSDSEYSK |
| | | VRLNRSSSSECSTVDNPLPGEGEEAEAEPMNSDEPE |
| | | ACFTDGCVRRFSCCQVNIESGKGKIWWNIRKTCYK |
| | | IVEHSWFESFIVLMILLSSGALAFEDIYIERKKTIKIIL |
| | | EYADKIFTYIFILEMLLKWIAYGYKTYFTNAWCWL |
| | | DFLIVDVSLVTLVANTLGYSDLGPIKSLRTLRALRP |
| | | LRALSRFEGMRVVVNALIGAIPSIMNVLLVCLIFWL |
| | | IFSIMGVNLFAGKFYECINTTDGSRFPASQVPNRSEC |
| | | FALMNVSQNVRWKNLKVNFDNVGLGYLSLLQVA |
| | | TFKGWTIIMYAAVDSVNVDKQPKYEYSLYMYIYF |
| | | VVFIIFGSFFTLNLFIGVIIDNFNQQKKKLGGQDIFM |
| | | TEEQKKYYNAMKKLGSKKPQKPIPRPGNKIQGCIF |
| | | DLVTNQAFDISIMVLICLNMVTMMVEKEGQSQHM |
| | | TEVLYWINVVFIILFTGECVLKLISLRHYYFTVGWNI |
| | | FDFVVVIISIVGMFLADLIETYFVSPTLFRVIRLARIG |
| | | RILRLVKGAKGIRTLLFALMMSLPALFNIGLLLFLV |
| | | MFIYAIFGMSNFAYVKKEDGINDMFNFETFGNSMI |
| | | CLFQITTSAGWDGLLAPILNSKPPDCDPKKVHPGSS |
| | | VEGDCGNPSVGIFYFVSYIIISFLVVVNMYIAVILEN |
| | | FSVATEESTEPLSEDDFEMFYEVWEKFDPDATQFIE |
| | | FSKLSDFAAALDPPLLIAKPNKVQLIAMDLPMVSG |
| | | DRIHCLDILFAFTKRVLGESGEMDSLRSQMEERFMS |
| | | ANPSKVSYEPITTTLKRKQEDVSATVIQRAYRRYRL |
| | | RQNVKNISSIYIKDGDRDDDLLNKKDMAFDNVNEN |
| | | SSPEKTDATSSTTSPPSYDSVTKPDKEKYEQDRTEK |
| | | EDKGKDSKESKK |
| 13 | huNav157 chimera 4 | MANFLLPRGTSSFRRFTRESLAAIEKRMAEKQARG |
| | | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYTTEFVDLGN |
| | | VSALRTFRVLRALKTISVISGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQANIEEA |
| | | KQKELEFQQMLDRLKKEQEEAEAIAAAAAEYTSIR |
| | | RSRIMGLSESSSETSKLSSKSAKERRNRRKKKNQKK |
| | | LSSGEEKGDAEKLSKSESEDSIRRKSFHLGVEGHRR |
| | | AHEKRLSTPNQSPLSIRGSLFSARRSSRTSLFSFKGR |
| | | GRDIGSETEFADDEHSIFGDNESRRGSLFVPHRPQE |
| | | RRSSNISQASRSPPMLPVNGKMHSAVDCNGVVSLV |
| | | DGRSALMLPNGQLLPEGTTNQIHKKRRCSSYLLSE |
| | | DMLNDPNLRQRAMSRASILTNTVEELEESRQKCPP |
| | | WWYRFAHKFLIWNCSPYWIKFKKCIYFIVMDPFVD |
| | | LAITICIVLNTLFMAMEHHPMTEEFKNVLAIGNLVF |
| | | TGIFAAEMVLKLIAMDPYEYFQVGWNIFDSLIVTLS |
| | | LVELFLADVEGLSVLRSFRLLRVFKLAKSWPTLNM |
| | | LIKIIGNSVGALGNLTLVLAIIVFIFAVVGMQLFGKS |
| | | YKECVCKINDDCTLPRWHMNDFFHSFLIVFRVLCG |
| | | EWIETMWDCMEVAGQAMCLIVYMMVMVIGNLVV |
| | | LNLFLALLLSSFSSDNLTAIEEDPDANNLQIAVTRIK |
| | | KGINYVKQTLREFILKAFSKKPKISREIRQAEDLNTK |
| | | KENYISNHTLAEMSKGHNFLKEKDKISGFGSSVDK |
| | | HLMEDSDGQSFIHNPSLTVTVPIAPGESDLENMNAE |
| | | ELSSDSDSEYSKVRLNRSSSSECSTVDNPLPGEGEE |
| | | AEAEPMNSDEPEACFTDGCVRRFSCCQVNIESGKG |
| | | KIWWNIRKTCYKIVEHSWFESFIVLMILLSSGALAF |
| | | EDIYIERKKTIKIILEYADKIFTYIFILEMLLKWIAYG |
| | | YKTYFTNAWCWLDFLIVDVSLVTLVANTLGYSDL |
| | | GPIKSLRTLRALRPLRALSRFEGMRVVVNALIGAIPS |
| | | IMNVLLVCLIFWLIFSIMGVNLFAGKFYECINTTDGS |
| | | RFPASQVPNRSECFALMNVSQNVRWKNLKVNFDN |
| | | VGLGYLSLLQVATFKGWTIIMYAAVDSVNVDKQP |
| | | KYEYSLYMYIYFVVFIIFGSFFTLNLFIGVIIDNFNQQ |
| | | KKKLGGQDIFMTEEQKKYYNAMKKLGSKKPQKPI |
| | | PRPGNKIQGCIFDLVTNQAFDISIMVLICLNMVTMM |
| | | VEKEGQSQHMTEVLYWINVVFIILFTGECVLKLISL |
| | | RHYYFTVGWNIFDFVVVIISIVGMFLADLIETYFVSP |
| | | TLFRVIRLARIGRILRLVKGAKGIRTLLFALMMSLP |
| | | ALFNIGLLLFLVMFIYAIFGMSNFAYVKKEDGINDM |
| | | FNFETFGNSMICLFQITTSAGWDGLLAPILNSKPPDC |
| | | DPKKVHPGSSVEGDCGNPSVGIFYFVSYIIISFLVVV |
| | | NMYIAVILENFSVATEESTEPLSEDDFEMFYEVWEK |
| | | FDPDATQFIEFSKLSDFAAALDPPLLIAKPNKVQLIA |
| | | MDLPMVSGDRIHCLDILFAFTKRVLGESGEMDSLR |
| | | SQMEERFMSANPSKVSYEPITTTLKRKQEDVSATVI |
| | | QRAYRRYRLRQNVKNISSIYIKDGDRDDDLLNKKD |
| | | MAFDNVNENSSPEKTDATSSTTSPPSYDSVTKPDKE |
| | | KYEQDRTEKEDKGKDSKESKK |
| 14 | huNav157 chimera 5 | MANFLLPRGTSSFRRFTRESLAAIEKRMAEKQARG |
| | | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYTTEFVDLGN |
| | | VSALRTFRVLRALKTISVISGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQATIAET |
| | | EEKEKRFQEAMEMLKKEHEALTIRGVDTVSRSSLE |
| | | MSPLAPVNSHERRSKRRKRMSSGTEECGEDRLPKS |
| | | DSEDGPRAMNHLSLTRGLSRTSMKPRSSRGSIFTFR |
| | | RRDLGSEADFADDENSTAGESESHHTSLLVPWPLR |
| | | RTSAQGQPSPGTSAPGHALHGKKNSTVDCNGVVSL |
| | | LGAGDPEATSPGSHLLRPVMLEHPPDTTTPSEEPGG |
| | | PQMLTSQAPCVDGFEEPGARQRALSAVSVLTSALE |
| | | ELEESRHKCPPCWNRLAQRYLIWECCPLWMSIKQG |
| | | VKLVVMDPFTDLTITMCIVLNTLFMALEHYNMTSE |
| | | FEEMLQVGNLVFTGIFTAEMTFKIIALDPYYYFQQG |
| | | WNIFDSIIVILSLMELGLSRMSNLSVLRSFRLLRVFK |
| | | LAKSWPTLNTLIKIIGNSVGALGNLTLVLAIIVFIFA |
| | | VVGMQLFGKNYSELRDSDSGLLPRWHMMDFFHAF |
| | | LIIFRILCGEWIETMWDCMEVSGQSLCLLVFLLVMV |
| | | IGNLVVLNLFLALLLSSFSADNLTAPDEDREMNNL |
| | | QLALARIQRGLRFVKRTTWDFCCGLLRQRPQKPAA |
| | | LAAQGQLPSCIATPYSPPPPETEKVPPTRKETRFEEG |
| | | EQPGQGTPGDPEPVCVPIAVAESDTDDQEEDEENSL |
| | | GTEEESSKQESQPVSGGPEAPPDSRTWSQVSATASS |
| | | EAEASASQADWRQQWKAEPQAPGCGETPEDSCSE |
| | | GSTADMTNTAELLEQIPDLGQDVKDPEDCFTEGCV |
| | | RRCPCCAVDTTQAPGKVWWRLRKTCYHIVEHSWF |
| | | ETFIIFMILLSSGALAFEDIYLEERKTIKVLLEYADK |
| | | MFTYVFVLEMLLKWVAYGFKKYFTNAWCWLDFL |
| | | IVDVSLVSLVANTLGFAEMGPIKSLRTLRALRPLRA |
| | | LSRFEGMRVVVNALVGAIPSIMNVLLVCLIFWLIFSI |
| | | MGVNLFAGKFGRCINQTEGDLPLNYTIVNNKSQCE |
| | | SLNLTGELYWTKVKVNFDNVGAGYLALLQVATFK |
| | | GWMDIMYAAVDSRGYEEQPQWEYNLYMYIYFVIF |
| | | IIFGSFFTLNLFIGVIIDNFNQQKKKLGGQDIFMTEEQ |
| | | KKYYNAMKKLGSKKPQKPIPRPGNKIQGCIFDLVT |
| | | NQAFDISIMVLICLNMVTMMVEKEGQSQHMTEVL |
| | | YWINVVFIILFTGECVLKLISLRHYYFTVGWNIFDFV |
| | | VVIISIVGMFLADLIETYFVSPTLFRVIRLARIGRILRL |
| | | VKGAKGIRTLLFALMMSLPALFNIGLLLFLVMFIYA |
| | | IFGMSNFAYVKKEDGINDMFNFETFGNSMICLFQIT |
| | | TSAGWDGLLAPILNSKPPDCDPKKVHPGSSVEGDC |
| | | GNPSVGIFYFVSYIIISFLVVVNMYIAVILENFSVATE |
| | | ESTEPLSEDDFEMFYEVWEKFDPDATQFIEFSKLSD |
| | | FAAALDPPLLIAKPNKVQLIAMDLPMVSGDRIHCL |
| | | DILFAFTKRVLGESGEMDSLRSQMEERFMSANPSK |
| | | VSYEPITTTLKRKQEDVSATVIQRAYRRYRLRQNV |
| | | KNISSIYIKDGDRDDDLLNKKDMAFDNVNENSSPE |
| | | KTDATSSTTSPPSYDSVTKPDKEKYEQDRTEKEDK |
| | | GKDSKESKK |
| 15 | huNav157 chimera 6 | MANFLLPRGTSSFRRFTRESLAAIEKRMAEKQARG |
| | | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYTTEFVDLGN |
| | | VSALRTFRVLRALKTISVISGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQATIAET |
| | | EEKEKRFQEAMEMLKKEHEALTIRGVDTVSRSSLE |
| | | MSPLAPVNSHERRSKRRKRMSSGTEECGEDRLPKS |
| | | DSEDGPRAMNHLSLTRGLSRTSMKPRSSRGSIFTFR |
| | | RRDLGSEADFADDENSTAGESESHHTSLLVPWPLR |
| | | RTSAQGQPSPGTSAPGHALHGKKNSTVDCNGVVSL |
| | | LGAGDPEATSPGSHLLRPVMLEHPPDTTTPSEEPGG |
| | | PQMLTSQAPCVDGFEEPGARQRALSAVSVLTSALE |
| | | ELEESRHKCPPCWNRLAQRYLIWECCPLWMSIKQG |
| | | VKLVVMDPFTDLTITMCIVLNTLFMALEHYNMTSE |
| | | FEEMLQVGNLVFTGIFTAEMTFKIIALDPYYYFQQG |
| | | WNIFDSIIVILSLMELGLSRMSNLSVLRSFRLLRVFK |
| | | LAKSWPTLNTLIKIIGNSVGALGNLTLVLAIIVFIFA |
| | | VVGMQLFGKNYSELRDSDSGLLPRWHMMDFFHAF |
| | | LIIFRILCGEWIETMWDCMEVSGQSLCLLVFLLVMV |
| | | IGNLVVLNLFLALLLSSFSSDNLTAIEEDPDANNLQI |
| | | AVTRIKKGINYVKQTLREFILKAFSKKPKISREIRQA |
| | | EDLNTKKENYISNHTLAEMSKGHNFLKEKDKISGF |
| | | GSSVDKHLMEDSDGQSFIHNPSLTVTVPIAPGESDL |
| | | ENMNAEELSSDSDSEYSKVRLNRSSSSECSTVDNPL |
| | | PGEGEEAEAEPMNSDEPEACFTDGCVRRFSCCQVN |
| | | IESGKGKIWWNIRKTCYKIVEHSWFESFIVLMILLSS |
| | | GALAFEDIYIERKKTIKIILEYADKIFTYIFILEMLLK |
| | | WIAYGYKTYFTNAWCWLDFLIVDVSLVTLVANTL |
| | | GYSDLGPIKSLRTLRALRPLRALSRFEGMRVVVNA |
| | | LIGAIPSIMNVLLVCLIFWLIFSIMGVNLFAGKFYECI |
| | | NTTDGSRFPASQVPNRSECFALMNVSQNVRWKNL |
| | | KVNFDNVGLGYLSLLQVATFKGWTIIMYAAVDSV |
| | | NVDKQPKYEYSLYMYIYFVVFIIFGSFFTLNLFIGVII |
| | | DNFNQQKKKLGGQDIFMTEEQKKYYNAMKKLGS |
| | | KKPQKPIPRPLNKYQGFIFDIVTKQAFDVTIMFLICL |
| | | NMVTMMVETDDQSPEKINILAKINLLEVAIFTGECI |
| | | VKLAALRHYYFTNSWNIFDFVVVILSIVGTVLSDIIQ |
| | | KYFFSPTLFRVIRLARIGRILRLIRGAKGIRTLLFALM |
| | | MSLPALFNIGLLLFLVMFIYSIFGMANFAYVKWEA |
| | | GIDDMFNFQTFANSMLCLFQITTSAGWDGLLSPILN |
| | | TGPPYCDPTLPNSNGSRGDCGSPAVGILFFTTYIIISF |
| | | LIVVNMYIAIILENFSVATEESTEPLSEDDFDMFYEI |
| | | WEKFDPEATQFIEYSVLSDFADALSEPLRIAKPNQIS |
| | | LINMDLPMVSGDRIHCMDILFAFTKRVLGESGEMD |
| | | ALKIQMEEKFMAANPSKISYEPITTTLRRKHEEVSA |
| | | MVIQRAFRRHLLQRSLKHASFLFRQQAGSGLSEED |
| | | APEREGLIAYVMSENFSRPLGPPSSSSISSTSFPPSYD |
| | | SVTRATSDNLQVRGSDYSHSEDLADFPPSPDRDRES |
| | | IV |
| 16 | huNav157 chimera 7 | MANFLLPRGTSSFRRFTRESLAAIEKRMAEKQARG |
| | | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYTTEFVDLGN |
| | | VSALRTFRVLRALKTISVISGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQANIEEA |
| | | KQKELEFQQMLDRLKKEQEEAEAIAAAAAEYTSIR |
| | | RSRIMGLSESSSETSKLSSKSAKERRNRRKKKNQKK |
| | | LSSGEEKGDAEKLSKSESEDSIRRKSFHLGVEGHRR |
| | | AHEKRLSTPNQSPLSIRGSLFSARRSSRTSLFSFKGR |
| | | GRDIGSETEFADDEHSIFGDNESRRGSLFVPHRPQE |
| | | RRSSNISQASRSPPMLPVNGKMHSAVDCNGVVSLV |
| | | DGRSALMLPNGQLLPEGTTNQIHKKRRCSSYLLSE |
| | | DMLNDPNLRQRAMSRASILTNTVEELEESRQKCPP |
| | | WWYRFAHKFLIWNCSPYWIKFKKCIYFIVMDPFVD |
| | | LAITICIVLNTLFMAMEHHPMTEEFKNVLAIGNLVF |
| | | TGIFAAEMVLKLIAMDPYEYFQVGWNIFDSLIVTLS |
| | | LVELFLADVEGLSVLRSFRLLRVFKLAKSWPTLNM |
| | | LIKIIGNSVGALGNLTLVLAIIVFIFAVVGMQLFGKS |
| | | YKECVCKINDDCTLPRWHMNDFFHSFLIVFRVLCG |
| | | EWIETMWDCMEVAGQAMCLIVYMMVMVIGNLVV |
| | | LNLFLALLLSSFSADNLTAPDEDREMNNLQLALARI |
| | | QRGLRFVKRTTWDFCCGLLRQRPQKPAALAAQGQ |
| | | LPSCIATPYSPPPPETEKVPPTRKETRFEEGEQPGQG |
| | | TPGDPEPVCVPIAVAESDTDDQEEDEENSLGTEEES |
| | | SKQESQPVSGGPEAPPDSRTWSQVSATASSEAEASA |
| | | SQADWRQQWKAEPQAPGCGETPEDSCSEGSTADM |
| | | TNTAELLEQIPDLGQDVKDPEDCFTEGCVRRCPCC |
| | | AVDTTQAPGKVWWRLRKTCYHIVEHSWFETFIIFM |
| | | ILLSSGALAFEDIYLEERKTIKVLLEYADKMFTYVF |
| | | VLEMLLKWVAYGFKKYFTNAWCWLDFLIVDVSL |
| | | VSLVANTLGFAEMGPIKSLRTLRALRPLRALSRFEG |
| | | MRVVVNALVGAIPSIMNVLLVCLIFWLIFSIMGVNL |
| | | FAGKFGRCINQTEGDLPLNYTIVNNKSQCESLNLTG |
| | | ELYWTKVKVNFDNVGAGYLALLQVATFKGWMDI |
| | | MYAAVDSRGYEEQPQWEYNLYMYIYFVIFIIFGSFF |
| | | TLNLFIGVIIDNFNQQKKKLGGQDIFMTEEQKKYYN |
| | | AMKKLGSKKPQKPIPRPLNKYQGFIFDIVTKQAFDV |
| | | TIMFLICLNMVTMMVETDDQSPEKINILAKINLLFV |
| | | AIFTGECIVKLAALRHYYFTNSWNIFDFVVVILSIVG |
| | | TVLSDIIQKYFFSPTLFRVIRLARIGRILRLIRGAKGIR |
| | | TLLFALMMSLPALFNIGLLLFLVMFIYSIFGMANFA |
| | | YVKWEAGIDDMFNFQTFANSMLCLFQITTSAGWD |
| | | GLLSPILNTGPPYCDPTLPNSNGSRGDCGSPAVGILF |
| | | FTTYIIISFLIVVNMYIAIILENFSVATEESTEPLSEDD |
| | | FDMFYEIWEKFDPEATQFIEYSVLSDFADALSEPLRI |
| | | AKPNQISLINMDLPMVSGDRIHCMDILFAFTKRVLG |
| | | ESGEMDALKIQMEEKFMAANPSKISYEPITTTLRRK |
| | | HEEVSAMVIQRAFRRHLLQRSLKHASFLFRQQAGS |
| | | GLSEEDAPEREGLIAYVMSENFSRPLGPPSSSSISSTS |
| | | FPPSYDSVTRATSDNLQVRGSDYSHSEDLADFPPSP |
| | | DRDRESIV |
| 17 | huNav157 chimera 8 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQATIAETEEKEKRFQEAMEMLK |
| | | KEHEALTIRGVDTVSRSSLEMSPLAPVNSHERRSKR |
| | | RKRMSSGTEECGEDRLPKSDSEDGPRAMNHLSLTR |
| | | GLSRTSMKPRSSRGSIFTFRRRDLGSEADFADDENS |
| | | TAGESESHHTSLLVPWPLRRTSAQGQPSPGTSAPGH |
| | | ALHGKKNSTVDCNGVVSLLGAGDPEATSPGSHLLR |
| | | PVMLEHPPDTTTPSEEPGGPQMLTSQAPCVDGFEEP |
| | | GARQRALSAVSVLTSALEELEESRHKCPPCWNRLA |
| | | QRYLIWECCPLWMSIKQGVKLVVMDPFTDLTITMC |
| | | IVLNTLFMALEHYNMTSEFEEMLQVGNLVFTGIFT |
| | | AEMTFKIIALDPYYYFQQGWNIFDSIIVILSLMELGL |
| | | SRMSNLSVLRSFRLLRVFKLAKSWPTLNTLIKIIGNS |
| | | VGALGNLTLVLAIIVFIFAVVGMQLFGKNYSELRDS |
| | | DSGLLPRWHMMDFFHAFLIIFRILCGEWIETMWDC |
| | | MEVSGQSLCLLVFLLVMVIGNLVVLNLFLALLLSSF |
| | | SADNLTAPDEDREMNNLQLALARIQRGLRFVKRTT |
| | | WDFCCGLLRQRPQKPAALAAQGQLPSCIATPYSPPP |
| | | PETEKVPPTRKETRFEEGEQPGQGTPGDPEPVCVPI |
| | | AVAESDTDDQEEDEENSLGTEEESSKQESQPVSGGP |
| | | EAPPDSRTWSQVSATASSEAEASASQADWRQQWK |
| | | AEPQAPGCGETPEDSCSEGSTADMINTAELLEQIPD |
| | | LGQDVKDPEDCFTEGCVRRCPCCAVDTTQAPGKV |
| | | WWRLRKTCYHIVEHSWFETFIIFMILLSSGALAFEDI |
| | | YLEERKTIKVLLEYADKMFTYVFVLEMLLKWVAY |
| | | GFKKYFTNAWCWLDFLIVDVSLVSLVANTLGFAE |
| | | MGPIKSLRTLRALRPLRALSRFEGMRVVVNALVGA |
| | | IPSIMNVLLVCLIFWLIFSIMGVNLFAGKFGRCINQT |
| | | EGDLPLNYTIVNNKSQCESLNLTGELYWTKVKVNF |
| | | DNVGAGYLALLQVATFKGWMDIMYAAVDSRGYE |
| | | EQPQWEYNLYMYIYFVIFIIFGSFFTLNLFIGVIIDNF |
| | | NQQKKKLGGQDIFMTEEQKKYYNAMKKLGSKKP |
| | | QKPIPRPLNKYQGFIFDIVTKQAFDVTIMFLICLNMV |
| | | TMMVETDDQSPEKINILAKINLLFVAIFTGECIVKLA |
| | | ALRHYYFTNSWNIFDFVVVILSIVGTVLSDIIQKYFF |
| | | SPTLFRVIRLARIGRILRLIRGAKGIRTLLFALMMSLP |
| | | ALFNIGLLLFLVMFIYSIFGMANFAYVKWEAGIDD |
| | | MFNFQTFANSMLCLFQITTSAGWDGLLSPILNTGPP |
| | | YCDPTLPNSNGSRGDCGSPAVGILFFTTYIIISFLIVV |
| | | NMYIAIILENFSVATEESTEPLSEDDFDMFYEIWEKF |
| | | DPEATQFIEYSVLSDFADALSEPLRIAKPNQISLINM |
| | | DLPMVSGDRIHCMDILFAFTKRVLGESGEMDALKI |
| | | QMEEKFMAANPSKISYEPITTTLRRKHEEVSAMVIQ |
| | | RAFRRHLLQRSLKHASFLFRQQAGSGLSEEDAPERE |
| | | GLIAYVMSENFSRPLGPPSSSSISSTSFPPSYDSVTRA |
| | | TSDNLQVRGSDYSHSEDLADFPPSPDRDRESIV |
| 18 | huNav157 chimera 12 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMVLTV |
| | | FCLSVFALIGLQLFMGNLRHKCVRNFTALNGTNGS |
| | | VEADGLVWESLDLYLSDPENYLLKNGTSDVLLCG |
| | | NSSDAGTCPEGYRCLKAGENPDHGYTSFDSFAWAF |
| | | LALFRLMTQDCWERLYQQTLRSAGKIYMIFFMLVI |
| | | FLGSFYLVNLILAVVAMAYEEQNQANIEEAKQKEL |
| | | EFQQMLDRLKKEQEEAEAIAAAAAEYTSIRRSRIM |
| | | GLSESSSETSKLSSKSAKERRNRRKKKNQKKLSSGE |
| | | EKGDAEKLSKSESEDSIRRKSFHLGVEGHRRAHEK |
| | | RLSTPNQSPLSIRGSLFSARRSSRTSLFSFKGRGRDIG |
| | | SETEFADDEHSIFGDNESRRGSLFVPHRPQERRSSNI |
| | | SQASRSPPMLPVNGKMHSAVDCNGVVSLVDGRSA |
| | | LMLPNGQLLPEGTTNQIHKKRRCSSYLLSEDMLND |
| | | PNLRQRAMSRASILTNTVEELEESRQKCPPWWYRF |
| | | AHKFLIWNCSPYWIKFKKCIYFIVMDPFVDLAITICI |
| | | VLNTLFMAMEHHPMTEEFKNVLAIGNLVFTGIFAA |
| | | EMVLKLIAMDPYEYFQVGWNIFDSLIVTLSLVELFL |
| | | ADVEGLSVLRSFRLLRVFKLAKSWPTLNMLIKIIGN |
| | | SVGALGNLTLVLAIIVFIFAVVGMQLFGKSYKECVC |
| | | KINDDCTLPRWHMNDFFHSFLIVFRVLCGEWIETM |
| | | WDCMEVAGQAMCLIVYMMVMVIGNLVVLNLFLA |
| | | LLLSSFSSDNLTAIEEDPDANNLQIAVTRIKKGINYV |
| | | KQTLREFILKAFSKKPKISREIRQAEDLNTKKENYIS |
| | | NHTLAEMSKGHNFLKEKDKISGFGSSVDKHLMEDS |
| | | DGQSFIHNPSLTVTVPIAPGESDLENMNAEELSSDS |
| | | DSEYSKVRLNRSSSSECSTVDNPLPGEGEEAEAEPM |
| | | NSDEPEACFTDGCVRRFSCCQVNIESGKGKIWWNI |
| | | RKTCYKIVEHSWFESFIVLMILLSSGALAFEDIYIER |
| | | KKTIKIILEYADKIFTYIFILEMLLKWIAYGYKTYFT |
| | | NAWCWLDFLIVDVSLVTLVANTLGYSDLGPIKSLR |
| | | TLRALRPLRALSRFEGMRVVVNALIGAIPSIMNVLL |
| | | VCLIFWLIFSIMGVNLFAGKFYECINTTDGSRFPASQ |
| | | VPNRSECFALMNVSQNVRWKNLKVNFDNVGLGY |
| | | LSLLQVATFKGWTIIMYAAVDSVNVDKQPKYEYSL |
| | | YMYIYFVVFIIFGSFFTLNLFIGVIIDNFNQQKKKLG |
| | | GQDIFMTEEQKKYYNAMKKLGSKKPQKPIPRPGNK |
| | | IQGCIFDLVTNQAFDISIMVLICLNMVTMMVEKEGQ |
| | | SQHMTEVLYWINVVFIILFTGECVLKLISLRHYYFT |
| | | VGWNIFDFVVVIISIVGMFLADLIETYFVSPTLFRVI |
| | | RLARIGRILRLVKGAKGIRTLLFALMMSLPALFNIG |
| | | LLLFLVMFIYAIFGMSNFAYVKKEDGINDMFNFETF |
| | | GNSMICLFQITTSAGWDGLLAPILNSKPPDCDPKKV |
| | | HPGSSVEGDCGNPSVGIFYFVSYIIISFLVVVNMYIA |
| | | VILENFSVATEESTEPLSEDDFEMFYEVWEKFDPDA |
| | | TQFIEFSKLSDFAAALDPPLLIAKPNKVQLIAMDLP |
| | | MVSGDRIHCLDILFAFTKRVLGESGEMDSLRSQME |
| | | ERFMSANPSKVSYEPITTTLKRKQEDVSATVIQRAY |
| | | RRYRLRQNVKNISSIYIKDGDRDDDLLNKKDMAFD |
| | | NVNENSSPEKTDATSSTTSPPSYDSVTKPDKEKYEQ |
| | | DRTEKEDKGKDSKESKK |
| 19 | huNav157 chimera 14 | MANFLLPRGTSSFRRFTRESLAAIEKRMAEKQARG |
| | | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFSMLIMCTILTNC |
| | | IFMTMNNPPDWTKNVEYTFTGIYTFESLVKILARGF |
| | | CLHAFTFLRDPWNWLDFVVIVFAYLTEFVNLGNVS |
| | | ALRTFRVLRALKTISVIPGLKTIVGALIQSVKKLADV |
| | | MILTVFCLSVFALIGLQLFMGNLKHKCFRNSLENNE |
| | | TLESIMNTLESEEDFRKYFYYLEGSKDALLCGFSTD |
| | | SGQCPEGYTCVKIGRNPDYGYTSFDTFSWAFLALF |
| | | RLMTQDYWENLYQQTLRAAGKTYMIFFVVVIFLG |
| | | SFYLINLILAVVAMAYEEQNQATIAETEEKEKRFQE |
| | | AMEMLKKEHEALTIRGVDTVSRSSLEMSPLAPVNS |
| | | HERRSKRRKRMSSGTEECGEDRLPKSDSEDGPRAM |
| | | NHLSLTRGLSRTSMKPRSSRGSIFTFRRRDLGSEAD |
| | | FADDENSTAGESESHHTSLLVPWPLRRTSAQGQPSP |
| | | GTSAPGHALHGKKNSTVDCNGVVSLLGAGDPEAT |
| | | SPGSHLLRPVMLEHPPDTTTPSEEPGGPQMLTSQAP |
| | | CVDGFEEPGARQRALSAVSVLTSALEELEESRHKCP |
| | | PCWNRLAQRYLIWECCPLWMSIKQGVKFIVMDPF |
| | | VDLAITICIVLNTLFMAMEHHPMTEEFKNVLAIGNL |
| | | VFTGIFAAEMVLKLIAMDPYYYFQQGWNIFDSLIVT |
| | | LSLVELFLADVEGLSVLRSFRLLRVFKLAKSWPTLN |
| | | TLIKIIGNSVGALGNLTLVLAIIVFIFAVVGMQLFGK |
| | | SYKECVCKINDDCTLPRWHMNDFFHSFLIVFRVLC |
| | | GEWIETMWDCMEVAGQAMCLIVYMMVMVIGNLV |
| | | VLNLFLALLLSSFSADNLTAPDEDREMNNLQLALA |
| | | RIQRGLRFVKRTTWDFCCGLLRQRPQKPAALAAQG |
| | | QLPSCIATPYSPPPPETEKVPPTRKETRFEEGEQPGQ |
| | | GTPGDPEPVCVPIAVAESDTDDQEEDEENSLGTEEE |
| | | SSKQESQPVSGGPEAPPDSRTWSQVSATASSEAEAS |
| | | ASQADWRQQWKAEPQAPGCGETPEDSCSEGSTAD |
| | | MTNTAELLEQIPDLGQDVKDPEDCFTEGCVRRCPC |
| | | CAVDTTQAPGKVWWRLRKTCYHIVEHSWFESFIVL |
| | | MILLSSGALAFEDIYIERKKTIKIILEYADKIFTYIFIL |
| | | EMLLKWIAYGYKKYFTNAWCWLDFLIVDVSLVTL |
| | | VANTLGYSDLGPIKSLRTLRALRPLRALSRFEGMRV |
| | | VVNALVGAIPSIMNVLLVCLIFWLIFSIMGVNLFAG |
| | | KFYECINTTDGSRFPASQVPNRSECFALMNVSQNV |
| | | RWKNLKVNFDNVGLGYLSLLQVATFKGWTIIMYA |
| | | AVDSVNVDKQPKYEYSLYMYIYFVVFIIFGSFFTLN |
| | | LFIGVIIDNFNQQKKKLGGQDIFMTEEQKKYYNAM |
| | | KKLGSKKPQKPIPRPLNKYQGFIFDLVTNQAFDISIM |
| | | VLICLNMVTMMVEKEGQSQHMTEVLYWINVVFIIL |
| | | FTGECVLKLISLRHYYFTVGWNIFDFVVVIISIVGMF |
| | | LADLIETYFVSPTLFRVIRLARIGRILRLVKGAKGIR |
| | | TLLFALMMSLPALFNIGLLLFLVMFIYAIFGMSNFA |
| | | YVKKEDGINDMFNFETFGNSMICLFQITTSAGWDG |
| | | LLAPILNSKPPDCDPKKVHPGSSVEGDCGNPSVGIF |
| | | YFVSYIIISFLVVVNMYIAVILENFSVATEESTEPLSE |
| | | DDFDMFYEIWEKFDPEATQFIEYSVLSDFADALSEP |
| | | LRIAKPNQISLINMDLPMVSGDRIHCMDILFAFTKR |
| | | VLGESGEMDALKIQMEEKFMAANPSKISYEPITTTL |
| | | RRKHEEVSAMVIQRAFRRHLLQRSLKHASFLFRQQ |
| | | AGSGLSEEDAPEREGLIAYVMSENFSRPLGPPSSSSI |
| | | SSTSFPPSYDSVTRATSDNLQVRGSDYSHSEDLADF |
| | | PPSPDRDRESIV |
| 20 | huNav157 chimera 14- | MANFLLPRGTSSFRRFTRESLAAIEKRMAEKQARG |
| | beta1-beta2-beta3 viral | STTLQESREGLPEEEAPRPQLDLQASKKLPDLYGNP |
| | P2A sequences italics; | PQELIGEPLEDLDPFYSTQKTFIVLNKGKTIFRFSAT |
| | beta1-beta2-beta3 are in | NALYVLSPFHPIRRAAVKILVHSLFSMLIMCTILTNC |
| | bold | IFMTMNNPPDWTKNVEYTFTGIYTFESLVKILARGF |
| | | CLHAFTFLRDPWNWLDFVVIVFAYLTEFVNLGNVS |
| | | ALRTFRVLRALKTISVIPGLKTIVGALIQSVKKLADV |
| | | MILTVFCLSVFALIGLQLFMGNLKHKCFRNSLENNE |
| | | TLESIMNTLESEEDFRKYFYYLEGSKDALLCGFSTD |
| | | SGQCPEGYTCVKIGRNPDYGYTSFDTFSWAFLALF |
| | | RLMTQDYWENLYQQTLRAAGKTYMIFFVVVIFLG |
| | | SFYLINLILAVVAMAYEEQNQATIAETEEKEKRFQE |
| | | AMEMLKKEHEALTIRGVDTVSRSSLEMSPLAPVNS |
| | | HERRSKRRKRMSSGTEECGEDRLPKSDSEDGPRAM |
| | | NHLSLTRGLSRTSMKPRSSRGSIFTFRRRDLGSEAD |
| | | FADDENSTAGESESHHTSLLVPWPLRRTSAQGQPSP |
| | | GTSAPGHALHGKKNSTVDCNGVVSLLGAGDPEAT |
| | | SPGSHLLRPVMLEHPPDTTTPSEEPGGPQMLTSQAP |
| | | CVDGFEEPGARQRALSAVSVLTSALEELEESRHKCP |
| | | PCWNRLAQRYLIWECCPLWMSIKQGVKFIVMDPF |
| | | VDLAITICIVLNTLFMAMEHHPMTEEFKNVLAIGNL |
| | | VFTGIFAAEMVLKLIAMDPYYYFQQGWNIFDSLIVT |
| | | LSLVELFLADVEGLSVLRSFRLLRVFKLAKSWPTLN |
| | | TLIKIIGNSVGALGNLTLVLAIIVFIFAVVGMQLFGK |
| | | SYKECVCKINDDCTLPRWHMNDFFHSFLIVFRVLC |
| | | GEWIETMWDCMEVAGQAMCLIVYMMVMVIGNLV |
| | | VLNLFLALLLSSFSADNLTAPDEDREMNNLQLALA |
| | | RIQRGLRFVKRTTWDFCCGLLRQRPQKPAALAAQG |
| | | QLPSCIATPYSPPPPETEKVPPTRKETRFEEGEQPGQ |
| | | GTPGDPEPVCVPIAVAESDTDDQEEDEENSLGTEEE |
| | | SSKQESQPVSGGPEAPPDSRTWSQVSATASSEAEAS |
| | | ASQADWRQQWKAEPQAPGCGETPEDSCSEGSTAD |
| | | MTNTAELLEQIPDLGQDVKDPEDCFTEGCVRRCPC |
| | | CAVDTTQAPGKVWWRLRKTCYHIVEHSWFESFIVL |
| | | MILLSSGALAFEDIYIERKKTIKIILEYADKIFTYIFIL |
| | | EMLLKWIAYGYKKYFTNAWCWLDFLIVDVSLVTL |
| | | VANTLGYSDLGPIKSLRTLRALRPLRALSRFEGMRV |
| | | VVNALVGAIPSIMNVLLVCLIFWLIFSIMGVNLFAG |
| | | KFYECINTTDGSRFPASQVPNRSECFALMNVSQNV |
| | | RWKNLKVNFDNVGLGYLSLLQVATFKGWTIIMYA |
| | | AVDSVNVDKQPKYEYSLYMYIYFVVFIIFGSFFTLN |
| | | LFIGVIIDNFNQQKKKLGGQDIFMTEEQKKYYNAM |
| | | KKLGSKKPQKPIPRPLNKYQGFIFDLVTNQAFDISIM |
| | | VLICLNMVTMMVEKEGQSQHMTEVLYWINVVFIIL |
| | | FTGECVLKLISLRHYYFTVGWNIFDFVVVIISIVGMF |
| | | LADLIETYFVSPTLFRVIRLARIGRILRLVKGAKGIR |
| | | TLLFALMMSLPALFNIGLLLFLVMFIYAIFGMSNFA |
| | | YVKKEDGINDMFNFETFGNSMICLFQITTSAGWDG |
| | | LLAPILNSKPPDCDPKKVHPGSSVEGDCGNPSVGIF |
| | | YFVSYIIISFLVVVNMYIAVILENFSVATEESTEPLSE |
| | | DDFDMFYEIWEKFDPEATQFIEYSVLSDFADALSEP |
| | | LRIAKPNQISLINMDLPMVSGDRIHCMDILFAFTKR |
| | | VLGESGEMDALKIQMEEKFMAANPSKISYEPITTTL |
| | | RRKHEEVSAMVIQRAFRRHLLQRSLKHASFLFRQQ |
| | | AGSGLSEEDAPEREGLIAYVMSENFSRPLGPPSSSSI |
| | | SSTSFPPSYDSVTRATSDNLQVRGSDYSHSEDLADF |
| | | PPSPDRDRESIVSGRGSGATNFSLLKQAGDVEENPGP |
| | | MGRLLALVVGAALVSSACGGCVEVDSETEAVY |
| | | GMTFKILCISCKRRSETNAETFTEWTFRQKGTE |
| | | EFVKILRYENEVLQLEEDERFEGRVVWNGSRGT |
| | | KDLQDLSIFITNVTYNHSGDYECHVYRLLFFENY |
| | | EHNTSVVKKIHIEVVDKANRDMASIVSEIMMYV |
| | | LIVVLTIWLVAEMIYCYKKIAAATETAAQENASE |
| | | YLAITSESKENCTGVQVAEGSGATNFSLLKQAGDV |
| | | EENPGPMHRDAWLPRPAFSLTGLSLFFSLVPPGR |
| | | SMEVTVPATLNVLNGSDARLPCTFNSCYTVNHK |
| | | QFSLNWTYQECNNCSEEMFLQFRMKIINLKLER |
| | | FQDRVEFSGNPSKYDVSVMLRNVQPEDEGIYNC |
| | | YIMNPPDRHRGHGKIHLQVLMEEPPERDSTVAV |
| | | IVGASVGGFLAVVILVLMVVKCVRRKKEQKLST |
| | | DDLKTEEEGKTDGEGNPDDGAKGSGATNFSLLKQ |
| | | AGDVEENPGPMPAFNRLFPLASLVLIYWVSVCFP |
| | | VCVEVPSETEAVQGNPMKLRCISCMKREEVEAT |
| | | TVVEWFYRPEGGKDFLIYEYRNGHQEVESPFQG |
| | | RLQWNGSKDLQDVSITVLNVTLNDSGLYTCNVS |
| | | REFEFEAHRPFVKTTRLIPLRVTEEAGEDFTSVV |
| | | SEIMMYILLVFLTLWLLIEMIYCYRKVSKAEEAA |
| | | QENASDYLAIPSENKENSAVPVEE |
| 21 | beta1-beta2-beta3 viral | MGRLLALVVGAALVSSACGGCVEVDSETEAVY |
| | P2A sequences italics; | GMTFKILCISCKRRSETNAETFTEWTFRQKGTE |
| | beta1-beta2-beta3 are in | EFVKILRYENEVLQLEEDERFEGRVVWNGSRGT |
| | bold | KDLQDLSIFITNVTYNHSGDYECHVYRLLFFENY |
| | | EHNTSVVKKIHIEVVDKANRDMASIVSEIMMYV |
| | | LIVVLTIWLVAEMIYCYKKIAAATETAAQENASE |
| | | YLAITSESKENCTGVQVAEGSGATNFSLLKQAGDV |
| | | EENPGPMHRDAWLPRPAFSLTGLSLFFSLVPPGR |
| | | SMEVTVPATLNVLNGSDARLPCTFNSCYTVNHK |
| | | QFSLNWTYQECNNCSEEMFLQFRMKIINLKLER |
| | | FQDRVEFSGNPSKYDVSVMLRNVQPEDEGIYNC |
| | | YIMNPPDRHRGHGKIHLQVLMEEPPERDSTVAV |
| | | IVGASVGGFLAVVILVLMVVKCVRRKKEQKLST |
| | | DDLKTEEEGKTDGEGNPDDGAKGSGATNFSLLKQ |
| | | AGDVEENPGPMPAFNRLFPLASLVLIYWVSVCFP |
| | | VCVEVPSETEAVQGNPMKLRCISCMKREEVEAT |
| | | TVVEWFYRPEGGKDFLIYEYRNGHQEVESPFQG |
| | | RLQWNGSKDLQDVSITVLNVTLNDSGLYTCNVS |
| | | REFEFEAHRPFVKTTRLIPLRVTEEAGEDFTSVV |
| | | SEIMMYILLVFLTLWLLIEMIYCYRKVSKAEEAA |
| | | QENASDYLAIPSENKENSAVPVEE |
| 22 | huNav1.5-beta1-beta2- | MANFLLPRGTSSFRRFTRESLAAIEKRMAEKQARG |
| | beta3 viral P2A | STTLQESREGLPEEEAPRPQLDLQASKKLPDLYGNP |
| | sequences italics; beta1- | PQELIGEPLEDLDPFYSTQKTFIVLNKGKTIFRFSAT |
| | beta2-beta3 are in bold | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYTTEFVDLGN |
| | | VSALRTFRVLRALKTISVISGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQATIAET |
| | | EEKEKRFQEAMEMLKKEHEALTIRGVDTVSRSSLE |
| | | MSPLAPVNSHERRSKRRKRMSSGTEECGEDRLPKS |
| | | DSEDGPRAMNHLSLTRGLSRTSMKPRSSRGSIFTFR |
| | | RRDLGSEADFADDENSTAGESESHHTSLLVPWPLR |
| | | RTSAQGQPSPGTSAPGHALHGKKNSTVDCNGVVSL |
| | | LGAGDPEATSPGSHLLRPVMLEHPPDTTTPSEEPGG |
| | | PQMLTSQAPCVDGFEEPGARQRALSAVSVLTSALE |
| | | ELEESRHKCPPCWNRLAQRYLIWECCPLWMSIKQG |
| | | VKLVVMDPFTDLTITMCIVLNTLFMALEHYNMTSE |
| | | FEEMLQVGNLVFTGIFTAEMTFKIIALDPYYYFQQG |
| | | WNIFDSIIVILSLMELGLSRMSNLSVLRSFRLLRVFK |
| | | LAKSWPTLNTLIKIIGNSVGALGNLTLVLAIIVFIFA |
| | | VVGMQLFGKNYSELRDSDSGLLPRWHMMDFFHAF |
| | | LIIFRILCGEWIETMWDCMEVSGQSLCLLVFLLVMV |
| | | IGNLVVLNLFLALLLSSFSADNLTAPDEDREMNNL |
| | | QLALARIQRGLRFVKRTTWDFCCGLLRQRPQKPAA |
| | | LAAQGQLPSCIATPYSPPPPETEKVPPTRKETRFEEG |
| | | EQPGQGTPGDPEPVCVPIAVAESDTDDQEEDEENSL |
| | | GTEEESSKQESQPVSGGPEAPPDSRTWSQVSATASS |
| | | EAEASASQADWRQQWKAEPQAPGCGETPEDSCSE |
| | | GSTADMTNTAELLEQIPDLGQDVKDPEDCFTEGCV |
| | | RRCPCCAVDTTQAPGKVWWRLRKTCYHIVEHSWF |
| | | ETFIIFMILLSSGALAFEDIYLEERKTIKVLLEYADK |
| | | MFTYVFVLEMLLKWVAYGFKKYFTNAWCWLDFL |
| | | IVDVSLVSLVANTLGFAEMGPIKSLRTLRALRPLRA |
| | | LSRFEGMRVVVNALVGAIPSIMNVLLVCLIFWLIFSI |
| | | MGVNLFAGKFGRCINQTEGDLPLNYTIVNNKSQCE |
| | | SLNLTGELYWTKVKVNFDNVGAGYLALLQVATFK |
| | | GWMDIMYAAVDSRGYEEQPQWEYNLYMYIYFVIF |
| | | IIFGSFFTLNLFIGVIIDNFNQQKKKLGGQDIFMTEEQ |
| | | KKYYNAMKKLGSKKPQKPIPRPLNKYQGFIFDIVT |
| | | KQAFDVTIMFLICLNMVTMMVETDDQSPEKINILA |
| | | KINLLEVAIFTGECIVKLAALRHYYFTNSWNIFDFV |
| | | VVILSIVGTVLSDIIQKYFFSPTLFRVIRLARIGRILRL |
| | | IRGAKGIRTLLFALMMSLPALFNIGLLLFLVMFIYSI |
| | | FGMANFAYVKWEAGIDDMFNFQTFANSMLCLFQI |
| | | TTSAGWDGLLSPILNTGPPYCDPTLPNSNGSRGDCG |
| | | SPAVGILFFTTYIIISFLIVVNMYIAIILENFSVATEEST |
| | | EPLSEDDFDMFYEIWEKFDPEATQFIEYSVLSDFAD |
| | | ALSEPLRIAKPNQISLINMDLPMVSGDRIHCMDILFA |
| | | FTKRVLGESGEMDALKIQMEEKFMAANPSKISYEPI |
| | | TTTLRRKHEEVSAMVIQRAFRRHLLQRSLKHASFLF |
| | | RQQAGSGLSEEDAPEREGLIAYVMSENFSRPLGPPS |
| | | SSSISSTSFPPSYDSVTRATSDNLQVRGSDYSHSEDL |
| | | ADFPPSPDRDRESIVSGRGSGATNFSLLKQAGDVEEN |
| | | PGPMGRLLALVVGAALVSSACGGCVEVDSETEA |
| | | VYGMTFKILCISCKRRSETNAETFTEWTFRQKG |
| | | TEEFVKILRYENEVLQLEEDERFEGRVVWNGSR |
| | | GTKDLQDLSIFITNVTYNHSGDYECHVYRLLFFE |
| | | NYEHNTSVVKKIHIEVVDKANRDMASIVSEIMM |
| | | YVLIVVLTIWLVAEMIYCYKKIAAATETAAQEN |
| | | ASEYLAITSESKENCTGVQVAEGSGATNFSLLKQA |
| | | GDVEENPGPMHRDAWLPRPAFSLTGLSLFFSLVP |
| | | PGRSMEVTVPATLNVLNGSDARLPCTFNSCYTV |
| | | NHKQFSLNWTYQECNNCSEEMFLQFRMKIINLK |
| | | LERFQDRVEFSGNPSKYDVSVMLRNVQPEDEGI |
| | | YNCYIMNPPDRHRGHGKIHLQVLMEEPPERDST |
| | | VAVIVGASVGGFLAVVILVLMVVKCVRRKKEQK |
| | | LSTDDLKTEEEGKTDGEGNPDDGAKGSGATNFSL |
| | | LKQAGDVEENPGPMPAFNRLFPLASLVLIYWVSV |
| | | CFPVCVEVPSETEAVQGNPMKLRCISCMKREEV |
| | | EATTVVEWFYRPEGGKDFLIYEYRNGHQEVESP |
| | | FQGRLQWNGSKDLQDVSITVLNVTLNDSGLYTC |
| | | NVSREFEFEAHRPFVKTTRLIPLRVTEEAGEDFT |
| | | SVVSEIMMYILLVFLTLWLLIEMIYCYRKVSKAE |
| | | EAAQENASDYLAIPSENKENSAVPVEE |
| 23 | huNav1.1 (alpha | MEQTVLVPPGPDSFNFFTRESLAAIERRIAEEKAKN |
| | subunit) | PKPDKKDDDENGPKPNSDLEAGKNLPFIYGDIPPEM |
| | | VSEPLEDLDPYYINKKTFIVLNKGKAIFRFSATSALY |
| | | ILTPFNPLRKIAIKILVHSLFSMLIMCTILTNCVFMTM |
| | | SNPPDWTKNVEYTFTGIYTFESLIKIIARGFCLEDFT |
| | | FLRDPWNWLDFTVITFAYVTEFVDLGNVSALRTFR |
| | | VLRALKTISVIPGLKTIVGALIQSVKKLSDVMILTVF |
| | | CLSVFALIGLQLFMGNLRNKCIQWPPTNASLEEHSI |
| | | EKNITVNYNGTLINETVFEFDWKSYIQDSRYHYFLE |
| | | GFLDALLCGNSSDAGQCPEGYMCVKAGRNPNYGY |
| | | TSFDTFSWAFLSLFRLMTQDFWENLYQLTLRAAGK |
| | | TYMIFFVLVIFLGSFYLINLILAVVAMAYEEQNQAT |
| | | LEEAEQKEAEFQQMIEQLKKQQEAAQQAATATAS |
| | | EHSREPSAAGRLSDSSSEASKLSSKSAKERRNRRKK |
| | | RKQKEQSGGEEKDEDEFQKSESEDSIRRKGFRFSIE |
| | | GNRLTYEKRYSSPHQSLLSIRGSLFSPRRNSRTSLFS |
| | | FRGRAKDVGSENDFADDEHSTFEDNESRRDSLFVP |
| | | RRHGERRNSNLSQTSRSSRMLAVFPANGKMHSTV |
| | | DCNGVVSLVGGPSVPTSPVGQLLPEVIIDKPATDDN |
| | | GTTTETEMRKRRSSSFHVSMDFLEDPSQRQRAMSI |
| | | ASILTNTVEELEESRQKCPPCWYKFSNIFLIWDCSPY |
| | | WLKVKHVVNLVVMDPFVDLAITICIVLNTLFMAM |
| | | EHYPMTDHFNNVLTVGNLVFTGIFTAEMFLKIIAM |
| | | DPYYYFQEGWNIFDGFIVTLSLVELGLANVEGLSVL |
| | | RSFRLLRVFKLAKSWPTLNMLIKIIGNSVGALGNLT |
| | | LVLAIIVFIFAVVGMQLFGKSYKDCVCKIASDCQLP |
| | | RWHMNDFFHSFLIVFRVLCGEWIETMWDCMEVAG |
| | | QAMCLTVFMMVMVIGNLVVLNLFLALLLSSFSAD |
| | | NLAATDDDNEMNNLQIAVDRMHKGVAYVKRKIY |
| | | EFIQQSFIRKQKILDEIKPLDDLNNKKDSCMSNHTA |
| | | EIGKDLDYLKDVNGTTSGIGTGSSVEKYIIDESDYM |
| | | SFINNPSLTVTVPIAVGESDFENLNTEDFSSESDLEES |
| | | KEKLNESSSSSEGSTVDIGAPVEEQPVVEPEETLEPE |
| | | ACFTEGCVQRFKCCQINVEEGRGKQWWNLRRTCF |
| | | RIVEHNWFETFIVFMILLSSGALAFEDIYIDQRKTIK |
| | | TMLEYADKVFTYIFILEMLLKWVAYGYQTYFTNA |
| | | WCWLDFLIVDVSLVSLTANALGYSELGAIKSLRTL |
| | | RALRPLRALSRFEGMRVVVNALLGAIPSIMNVLLV |
| | | CLIFWLIFSIMGVNLFAGKFYHCINTTTGDRFDIEDV |
| | | NNHTDCLKLIERNETARWKNVKVNFDNVGFGYLS |
| | | LLQVATFKGWMDIMYAAVDSRNVELQPKYEESLY |
| | | MYLYFVIFIIFGSFFTLNLFIGVIIDNFNQQKKKFGGQ |
| | | DIFMTEEQKKYYNAMKKLGSKKPQKPIPRPGNKFQ |
| | | GMVFDFVTRQVFDISIMILICLNMVTMMVETDDQS |
| | | EYVTTILSRINLVFIVLFTGECVLKLISLRHYYFTIG |
| | | WNIFDFVVVILSIVGMFLAELIEKYFVSPTLFRVIRL |
| | | ARIGRILRLIKGAKGIRTLLFALMMSLPALFNIGLLL |
| | | FLVMFIYAIFGMSNFAYVKREVGIDDMFNFETFGNS |
| | | MICLFQITTSAGWDGLLAPILNSKPPDCDPNKVNPG |
| | | SSVKGDCGNPSVGIFFFVSYIIISFLVVVNMYIAVILE |
| | | NFSVATEESAEPLSEDDFEMFYEVWEKFDPDATQF |
| | | MEFEKLSQFAAALEPPLNLPQPNKLQLIAMDLPMV |
| | | SGDRIHCLDILFAFTKRVLGESGEMDALRIQMEERF |
| | | MASNPSKVSYQPITTTLKRKQEEVSAVIIQRAYRRH |
| | | LLKRTVKQASFTYNKNKIKGGANLLIKEDMIIDRIN |
| | | ENSITEKTDLTMSTAACPPSYDRVTKPIVEKHEQEG |
| | | KDEKAKGK |
| 24 | huNav1.2 (alpha | MAQSVLVPPGPDSFRFFTRESLAAIEQRIAEEKAKR |
| | subunit) | PKQERKDEDDENGPKPNSDLEAGKSLPFIYGDIPPE |
| | | MVSVPLEDLDPYYINKKTFIVLNKGKAISRFSATPA |
| | | LYILTPFNPIRKLAIKILVHSLFNMLIMCTILTNCVFM |
| | | TMSNPPDWTKNVEYTFTGIYTFESLIKILARGFCLE |
| | | DFTFLRDPWNWLDFTVITFAYVTEFVDLGNVSALR |
| | | TFRVLRALKTISVIPGLKTIVGALIQSVKKLSDVMIL |
| | | TVFCLSVFALIGLQLFMGNLRNKCLQWPPDNSSFEI |
| | | NITSFFNNSLDGNGTTFNRTVSIFNWDEYIEDKSHF |
| | | YFLEGQNDALLCGNSSDAGQCPEGYICVKAGRNPN |
| | | YGYTSFDTFSWAFLSLFRLMTQDFWENLYQLTLRA |
| | | AGKTYMIFFVLVIFLGSFYLINLILAVVAMAYEEQN |
| | | QATLEEAEQKEAEFQQMLEQLKKQQEEAQAAAAA |
| | | ASAESRDFSGAGGIGVFSESSSVASKLSSKSEKELK |
| | | NRRKKKKQKEQSGEEEKNDRVRKSESEDSIRRKGF |
| | | RFSLEGSRLTYEKRFSSPHQSLLSIRGSLFSPRRNSR |
| | | ASLFSFRGRAKDIGSENDFADDEHSTFEDNDSRRDS |
| | | LFVPHRHGERRHSNVSQASRASRVLPILPMNGKMH |
| | | SAVDCNGVVSLVGGPSTLTSAGQLLPEGTTTETEIR |
| | | KRRSSSYHVSMDLLEDPTSRQRAMSIASILTNTMEE |
| | | LEESRQKCPPCWYKFANMCLIWDCCKPWLKVKHL |
| | | VNLVVMDPFVDLAITICIVLNTLFMAMEHYPMTEQ |
| | | FSSVLSVGNLVFTGIFTAEMFLKIIAMDPYYYFQEG |
| | | WNIFDGFIVSLSLMELGLANVEGLSVLRSFRLLRVF |
| | | KLAKSWPTLNMLIKIIGNSVGALGNLTLVLAIIVFIF |
| | | AVVGMQLFGKSYKECVCKISNDCELPRWHMHDFF |
| | | HSFLIVFRVLCGEWIETMWDCMEVAGQTMCLTVF |
| | | MMVMVIGNLVVLNLFLALLLSSFSSDNLAATDDD |
| | | NEMNNLQIAVGRMQKGIDFVKRKIREFIQKAFVRK |
| | | QKALDEIKPLEDLNNKKDSCISNHTTIEIGKDLNYL |
| | | KDGNGTTSGIGSSVEKYVVDESDYMSFINNPSLTVT |
| | | VPIAVGESDFENLNTEEFSSESDMEESKEKLNATSSS |
| | | EGSTVDIGAPAEGEQPEVEPEESLEPEACFTEDCVR |
| | | KFKCCQISIEEGKGKLWWNLRKTCYKIVEHNWFET |
| | | FIVFMILLSSGALAFEDIYIEQRKTIKTMLEYADKVF |
| | | TYIFILEMLLKWVAYGFQVYFTNAWCWLDFLIVDV |
| | | SLVSLTANALGYSELGAIKSLRTLRALRPLRALSRF |
| | | EGMRVVVNALLGAIPSIMNVLLVCLIFWLIFSIMGV |
| | | NLFAGKFYHCINYTTGEMFDVSVVNNYSECKALIE |
| | | SNQTARWKNVKVNFDNVGLGYLSLLQVATFKGW |
| | | MDIMYAAVDSRNVELQPKYEDNLYMYLYFVIFIIF |
| | | GSFFTLNLFIGVIIDNFNQQKKKFGGQDIFMTEEQK |
| | | KYYNAMKKLGSKKPQKPIPRPANKFQGMVFDFVT |
| | | KQVFDISIMILICLNMVTMMVETDDQSQEMTNILY |
| | | WINLVFIVLFTGECVLKLISLRYYYFTIGWNIFDFVV |
| | | VILSIVGMFLAELIEKYFVSPTLFRVIRLARIGRILRLI |
| | | KGAKGIRTLLFALMMSLPALFNIGLLLFLVMFIYAIF |
| | | GMSNFAYVKREVGIDDMFNFETFGNSMICLFQITTS |
| | | AGWDGLLAPILNSGPPDCDPDKDHPGSSVKGDCGN |
| | | PSVGIFFFVSYIIISFLVVVNMYIAVILENFSVATEES |
| | | AEPLSEDDFEMFYEVWEKFDPDATQFIEFAKLSDFA |
| | | DALDPPLLIAKPNKVQLIAMDLPMVSGDRIHCLDIL |
| | | FAFTKRVLGESGEMDALRIQMEERFMASNPSKVSY |
| | | EPITTTLKRKQEEVSAIIIQRAYRRYLLKQKVKKVSS |
| | | IYKKDKGKECDGTPIKEDTLIDKLNENSTPEKTDMT |
| | | PSTTSPPSYDSVTKPEKEKFEKDKSEKEDKGKDIRE |
| | | SKK |
| 25 | huNav1.3 (alpha | MAQALLVPPGPESFRLFTRESLAAIEKRAAEEKAKK |
| | subunit) | PKKEQDNDDENKPKPNSDLEAGKNLPFIYGDIPPE |
| | | MVSEPLEDLDPYYINKKTFIVMNKGKAIFRFSATSA |
| | | LYILTPLNPVRKIAIKILVHSLFSMLIMCTILTNCVFM |
| | | TLSNPPDWTKNVEYTFTGIYTFESLIKILARGFCLED |
| | | FTFLRDPWNWLDFSVIVMAYVTEFVSLGNVSALRT |
| | | FRVLRALKTISVIPGLKTIVGALIQSVKKLSDVMILT |
| | | VFCLSVFALIGLQLFMGNLRNKCLQWPPSDSAFET |
| | | NTTSYFNGTMDSNGTFVNVTMSTFNWKDYIGDDS |
| | | HFYVLDGQKDPLLCGNGSDAGQCPEGY |
| | | ICVKAGRNPNYGYTSFDTFSWAFLSLFRLMTQDYW |
| | | ENLYQLTLRAAGKTYMIFFVLVIFLGSFYLVNLILA |
| | | VVAMAYEEQNQATLEEAEQKEAEFQQMLEQLKK |
| | | QQEEAQAVAAASAASRDFSGIGGLGELLESSSEASK |
| | | LSSKSAKEWRNRRKKRRQREHLEGNNKGERDSFP |
| | | KSESEDSVKRSSFLFSMDGNRLTSDKKFCSPHQSLL |
| | | SIRGSLFSPRRNSKTSIFSFRGRAKDVGSENDFADDE |
| | | HSTFEDSESRRDSLFVPHRHGERRNSNVSQASMSSR |
| | | MVPGLPANGKMHSTVDCNGVVSLVGGPSALTSPT |
| | | GQLPPEGTTTETEVRKRRLSSYQISMEMLEDSSGRQ |
| | | RAVSIASILTNTMEELEESRQKCPPCWYRFANVFLI |
| | | WDCCDAWLKVKHLVNLIVMDPFVDLAITICI |
| | | VLNTLFMAMEHYPMTEQFSSVLTVGNLVFTGIFTA |
| | | EMVLKIIAMDPYYYFQEGWNIFDGIIVSLSLMELGL |
| | | SNVEGLSVLRSFRLLRVFKLAKSWPTLNMLIKIIGN |
| | | SVGALGNLTLVLAIIVFIFAVVGMQLFGKSYKECVC |
| | | KINDDCTLPRWHMNDFFHSFLIVFRVLCGEWIETM |
| | | WDCMEVAGQTMCLIVFMLVMVIGNLVVLNLFLAL |
| | | LLSSFSSDNLAATDDDNEMNNLQIAVGRMQKGIDY |
| | | VKNKMRECFQKAFFRKPKVIEIHEGNKIDSCMSNN |
| | | TGIEISKELNYLRDGNGTTSGVGTGSSVEKYVIDEN |
| | | DYMSFINNPSLTVTVPIAVGESDFENLNTEEFSSESE |
| | | LEESKEKLNATSSSEGSTVDVVLPREGEQAETEPEE |
| | | DLKPEACFTEGCIKKFPFCQVSTEEGKGK |
| | | IWWNLRKTCYSIVEHNWFETFIVFMILLSSGALAFE |
| | | DIYIEQRKTIKTMLEYADKVFTYIFILEMLLKWVAY |
| | | GFQTYFTNAWCWLDFLIVDVSLVSLVANALGYSEL |
| | | GAIKSLRTLRALRPLRALSRFEGMRVVVNALVGAIP |
| | | SIMNVLLVCLIFWLIFSIMGVNLFAGKFYHCVNMTT |
| | | GNMFDISDVNNLSDCQALGKQARWKNVKVNFDN |
| | | VGAGYLALLQVATFKGWMDIMYAAVDSRDVKLQ |
| | | PVYEENLYMYLYFVIFIIFGSFFTLNLFIGVIIDNFNQ |
| | | QKKKFGGQDIFMTEEQKKYYNAMKKLGSKKPQKP |
| | | IPRPANKFQGMVFDFVTRQVFDISIMILICLNMVTM |
| | | MVETDDQGKYMTLVLSRINLVFIVLFTGEFVLKLV |
| | | SLRHYYFTIGWNIFDFVVVILSIVGMFLAEMI |
| | | EKYFVSPTLFRVIRLARIGRILRLIKGAKGIRTLLFAL |
| | | MMSLPALFNIGLLLFLVMFIYAIFGMSNFAYVKKE |
| | | AGIDDMFNFETFGNSMICLFQITTSAGWDGLLAPIL |
| | | NSAPPDCDPDTIHPGSSVKGDCGNPSVGIFFFVSYIII |
| | | SFLVVVNMYIAVILENFSVATEESAEPLSEDDFEMF |
| | | YEVWEKFDPDATQFIEFSKLSDFAAALDPPLLIAKP |
| | | NKVQLIAMDLPMVSGDRIHCLDILFAFTKRVLGES |
| | | GEMDALRIQMEDRFMASNPSKVSYEPITTTLKRKQ |
| | | EEVSAAIIQRNFRCYLLKQRLKNISSNYNKEAIKGRI |
| | | DLPIKQDMIIDKLNGNSTPEKTDGSSSTTSPPSYDSV |
| | | TKPDKEKFEKDKPEKESKGKEVRENQK |
| 26 | huNav1.4 (alpha | MARPSLCTLVPLGPECLRPFTRESLAAIEQRAVEEE |
| | subunit) | ARLQRNKQMEIEEPERKPRSDLEAGKNLPMIYGDP |
| | | PPEVIGIPLEDLDPYYSNKKTFIVLNKGKAIFRFSAT |
| | | PALYLLSPFSVVRRGAIKVLIHALFSMFIMITILTNC |
| | | VFMTMSDPPPWSKNVEYTFTGIYTFESLIKILARGF |
| | | CVDDFTFLRDPWNWLDFSVIMMAYLTEFVDLGNIS |
| | | ALRTFRVLRALKTITVIPGLKTIVGALIQSVKKLSDV |
| | | MILTVFCLSVFALVGLQLFMGNLRQKCVRWPPPFN |
| | | DTNTTWYSNDTWYGNDTWYGNEMWYGNDSWY |
| | | ANDTWNSHASWATNDTFDWDAYISDEGNFYFLEG |
| | | SNDALLCGNSSDAGHCPEGYECIKTGRNPNYGYTS |
| | | YDTFSWAFLALFRLMTQDYWENLFQLTLRAAGKT |
| | | YMIFFVVIIFLGSFYLINLILAVVAMAYAEQNEATL |
| | | AEDKEKEEEFQQMLEKFKKHQEELEKAKAAQALE |
| | | GGEADGDPAHGKDCNGSLDTSQGEKGAPRQSSSG |
| | | DSGISDAMEELEEAHQKCPPWWYKCAHKVLIWNC |
| | | CAPWLKFKNIIHLIVMDPFVDLGITICIVLNTLFMA |
| | | MEHYPMTEHFDNVLTVGNLVFTGIFTAEMVLKLIA |
| | | MDPYEYFQQGWNIFDSIIVTLSLVELGLANVQGLSV |
| | | LRSFRLLRVFKLAKSWPTLNMLIKIIGNSVGALGNL |
| | | TLVLAIIVFIFAVVGMQLFGKSYKECVCKIALDCNL |
| | | PRWHMHDFFHSFLIVFRILCGEWIETMWDCMEVA |
| | | GQAMCLTVFLMVMVIGNLVVLNLFLALLLSSFSAD |
| | | SLAASDEDGEMNNLQIAIGRIKLGIGFAKAFLLGLL |
| | | HGKILSPKDIMLSLGEADGAGEAGEAGETAPEDEK |
| | | KEPPEEDLKKDNHILNHMGLADGPPSSLELDHLNFI |
| | | NNPYLTIQVPIASEESDLEMPTEEETDTFSEPEDSKK |
| | | PPQPLYDGNSSVCSTADYKPPEEDPEEQAEENPEGE |
| | | QPEECFTEACVQRWPCLYVDISQGRGKKWWTLRR |
| | | ACFKIVEHNWFETFIVFMILLSSGALAFEDIYIEQRR |
| | | VIRTILEYADKVFTYIFIMEMLLKWVAYGFKVYFT |
| | | NAWCWLDFLIVDVSIISLVANWLGYSELGPIKSLRT |
| | | LRALRPLRALSRFEGMRVVVNALLGAIPSIMNVLL |
| | | VCLIFWLIFSIMGVNLFAGKFYYCINTTTSERFDISE |
| | | VNNKSECESLMHTGQVRWLNVKVNYDNVGLGYL |
| | | SLLQVATFKGWMDIMYAAVDSREKEEQPQYEVNL |
| | | YMYLYFVIFIIFGSFFTLNLFIGVIIDNFNQQKKKLG |
| | | GKDIFMTEEQKKYYNAMKKLGSKKPQKPIPRPQNK |
| | | IQGMVYDLVTKQAFDITIMILICLNMVTMMVETDN |
| | | QSQLKVDILYNINMIFIIIFTGECVLKMLALRQYYFT |
| | | VGWNIFDFVVVILSIVGLALSDLIQKYFVSPTLFRVI |
| | | RLARIGRVLRLIRGAKGIRTLLFALMMSLPALFNIG |
| | | LLLFLVMFIYSIFGMSNFAYVKKESGIDDMFNFETF |
| | | GNSIICLFEITTSAGWDGLLNPILNSGPPDCDPNLEN |
| | | PGTSVKGDCGNPSIGICFFCSYIIISFLIVVNMYIAIIL |
| | | ENFNVATEESSEPLGEDDFEMFYETWEKFDPDATQ |
| | | FIAYSRLSDFVDTLQEPLRIAKPNKIKLITLDLPMVP |
| | | GDKIHCLDILFALTKEVLGDSGEMDALKQTMEEKF |
| | | MAANPSKVSYEPITTTLKRKHEEVCAIKIQRAYRRH |
| | | LLQRSMKQASYMYRHSHDGSGDDAPEKEGLLANT |
| | | MSKMYGHENGNSSSPSPEEKGEAGDAGPTMGLMP |
| | | ISPSDTAWPPAPPPGQTVRPGVKESLV |
| 27 | huNav1.5 (alpha | MANFLLPRGTSSFRRFTRESLAAIEKRMAEKQARG |
| | subunit) | STTLQESREGLPEEEAPRPQLDLQASKKLPDLYGNP |
| | | PQELIGEPLEDLDPFYSTQKTFIVLNKGKTIFRFSAT |
| | | NALYVLSPFHPIRRAAVKILVHSLFNMLIMCTILTN |
| | | CVFMAQHDPPPWTKYVEYTFTAIYTFESLVKILAR |
| | | GFCLHAFTFLRDPWNWLDFSVIIMAYVSENIKLGN |
| | | LSALRTFRVLRALKTISVIPGLKTIVGALIQSVKKLA |
| | | DVMVLTVFCLSVFALIGLQLFMGNLRHKCVRNFTA |
| | | LNGTNGSVEADGLVWESLDLYLSDPENYLLKNGTS |
| | | DVLLCGNSSDAGTCPEGYRCLKAGENPDHGYTSFD |
| | | SFAWAFLALFRLMTQDCWERLYQQTLRSAGKIYMI |
| | | FFMLVIFLGSFYLVNLILAVVAMAYEEQNQATIAET |
| | | EEKEKRFQEAMEMLKKEHEALTIRGVDTVSRSSLE |
| | | MSPLAPVNSHERRSKRRKRMSSGTEECGEDRLPKS |
| | | DSEDGPRAMNHLSLTRGLSRTSMKPRSSRGSIFTFR |
| | | RRDLGSEADFADDENSTAGESESHRTSLLVPWPLR |
| | | RTSAQGQPSPGTSAPGHALHGKKNSTVDCNGVVSL |
| | | LGAGDPEATSPGSHLLRPVMLEHPPDTTTPSEEPGG |
| | | PQMLTSQAPCVDGFEEPGARQRALSAVSVLTSALE |
| | | ELEESRHKCPPCWNRLAQRYLIWECCPLWMSIKQG |
| | | VKLVVMDPFTDLTITMCIVLNTLFMALEHYNMTSE |
| | | FEEMLQVGNLVFTGIFTAEMTFKIIA |
| | | LDPYYYFQQGWNIFDSIIVILSLMELGLSRMSNLSV |
| | | LRSFRLLRVFKLAKSWPTLNTLIKIIGNSVGALGNL |
| | | TLVLAIIVFIFAVVGMQLFGKNYSELRDSDSGLLPR |
| | | WHMMDFFHAFLIIFRILCGEWIETMWDCMEVSGQS |
| | | LCLLVFLLVMVIGNLVVLNLFLALLLSSFSADNLTA |
| | | PDEDREMNNLQLALARIQRGLRFVKRTTWDFCCG |
| | | LLRQRPQKPAALAAQGQLPSCIATPYSPPPPETEKV |
| | | PPTRKETRFEEGEQPGQGTPGDPEPVCVPIAVAESD |
| | | TDDQEEDEENSLGTEEESSKQESQPVSGGPEAPPDS |
| | | RTWSQVSATASSEAEASASQADWRQQWKAEPQAP |
| | | GCGETPEDSCSEGSTADMTNTAELLEQIPDLGQDV |
| | | KDPEDCFTEGCVRRCPCCAVDTTQAPGKVWWRLR |
| | | KTCYHIVEHSWFETFIIFMILLSSGALAFEDIYLEER |
| | | KTIKVLLEYADKMFTYVFVLEMLLKWVAYGFKKY |
| | | FTNAWCWLDFLIVDVSLVSLVANTLGFAEMGPIKS |
| | | LRTLRALRPLRALSRFEGMRVVVNALVGAIPSIMN |
| | | VLLVCLIFWLIFSIMGVNLFAGKFGRCINQTEGDLP |
| | | LNYTIVNNKSQCESLNLTGELYWTKVKVNFDNVG |
| | | AGYLALLQVATFKGWMDIMYAAVDSRGYEEQPQ |
| | | WEYNLYMYIYFVIFIIFGSFFTLNLFIGVIIDNFNQQK |
| | | KKLGGQDIFMTEEQKKYYNAMKKLGSKKPQKPIP |
| | | RPLNKYQGFIFDIVTKQAFDVTIMFLICLN |
| | | MVTMMVETDDQSPEKINILAKINLLFVAIFTGTVLS |
| | | DIIQKYFFSPTLFRVIRLARIGRILRLIRGAKGIRTLLF |
| | | ALMMSLPALFNIGLLLFLVMFIYSIFGMANFAYVK |
| | | WEAGIDDMFNFQTFANSMLCLFQITTSAGWDGLLS |
| | | PILNTGPPYCDPTLPNSNGSRGDCGSPAVGILFFTTY |
| | | IIISFLIVVNMYIAIILENFSVATEESTEPLSEDDFDMF |
| | | YEIWEKFDPEATQFIEYSVLSDFADALSEPLRIAKPN |
| | | QISLINMDLPMVSGDRIHCMDI |
| | | LFAFTKRVLGESGEMDALKIQMEEKFMAANPSKIS |
| | | YEPITTTLRRKHEEVSAMVIQRAFRRHLLQRSLKHA |
| | | SFLFRQQAGSGLSEEDAPEREGLIAYVMSENFSRPL |
| | | GPPSSSSISSTSFPPSYDSVTRATSDNLQVRGSDYSH |
| | | SEDLADFPPSPDRDRESIV |
| 28 | huNav1.6 (alpha | MAARLLAPPGPDSFKPFTPESLANIERRIAESKLKKP |
| | subunit) | PKADGSHREDDEDSKPKPNSDLEAGKSLPFIYGDIP |
| | | QGLVAVPLEDFDPYYLTQKTFVVLNRGKTLFRFSA |
| | | TPALYILSPFNLIRRIAIKILIHSVFSMIIMCTILTNCVF |
| | | MTFSNPPDWSKNVEYTFTGIYTFESLVKIIARGFCID |
| | | GFTFLRDPWNWLDFSVIMMAYITEFVNLGNVSALR |
| | | TFRVLRALKTISVIPGLKTIVGALIQSVKKLSDVMIL |
| | | TVFCLSVFALIGLQLFMGNLRNKCVVWPINFNESY |
| | | LENGTKGFDWEEYINNKTNFYTVPGMLEPLLCGNS |
| | | SDAGQCPEGYQCMKAGRNPNYGYTSFDTFSWAFL |
| | | ALFRLMTQDYWENLYQLTLRAAGKTYMIFFVLVIF |
| | | VGSFYLVNLILAVVAMAYEEQNQATLEEAEQKEA |
| | | EFKAMLEQLKKQQEEAQAAAMATSAGTVSEDAIE |
| | | EEGEEGGGSPRSSSEISKLSSKSAKERRNRRKKRKQ |
| | | KELSEGEEKGDPEKVFKSESEDGMRRKAFRLPDNR |
| | | IGRKFSIMNQSLLSIPGSPFLSRHNSKSSIFSFRGPGR |
| | | FRDPGSENEFADDEHSTVEESEGRRDSLFIPIRARER |
| | | RSSYSGYSGYSQGSRSSRIFPSLRRSVKRNSTVDCN |
| | | GVVSLIGGPGSHIGGRLLPEATTEVEIKKKGPGSLL |
| | | VSMDQLASYGRKDRINSIMSVVTNTLVEELEESQR |
| | | KCPPCWYKFANTFLIWECHPYWIKLKEIVNLIVMD |
| | | PFVDLAITICIVLNTLFMAMEHHPMTPQFEHVLAVG |
| | | NLVFTGIFTAEMFLKLIAMDPYYYFQEGWNIFDGFI |
| | | VSLSLMELSLADVEGLSVLRSFRLLRVFKLAKSWP |
| | | TLNMLIKIIGNSVGALGNLTLVLAIIVFIFAVVGMQL |
| | | FGKSYKECVCKINQDCELPRWHMHDFFHSFLIVFR |
| | | VLCGEWIETMWDCMEVAGQAMCLIVFMMVMVIG |
| | | NLVVLNLFLALLLSSFSADNLAATDDDGEMNNLQI |
| | | SVIRIKKGVAWTKLKVHAFMQAHFKQREADEVKP |
| | | LDELYEKKANCIANHTGADIHRNGDFQKNGNGTTS |
| | | GIGSSVEKYIIDEDHMSFINNPNLTVRVPIAVGESDF |
| | | ENLNTEDVSSESDPEGSKDKLDDTSSSEGSTIDIKPE |
| | | VEEVPVEQPEEYLDPDACFTEGCVQRFKCCQVNIE |
| | | EGLGKSWWILRKTCFLIVEHNWFETFIIFMILLSSGA |
| | | LAFEDIYIEQRKTIRTILEYADKVFTYIFILEMLLKW |
| | | TAYGFVKFFTNAWCWLDFLIVAVSLVSLIANALGY |
| | | SELGAIKSLRTLRALRPLRALSRFEGMRVVVNALV |
| | | GAIPSIMNVLLVCLIFWLIFSIMGVNLFAGKYHYCF |
| | | NETSEIRFEIEDVNNKTECEKLMEGNNTEIRWKNV |
| | | KINFDNVGAGYLALLQVATFKGWMDIMYAAVDSR |
| | | KPDEQPKYEDNIYMYIYFVIFIIFGSFFTLNLFIGVIID |
| | | NFNQQKKKFGGQDIFMTEEQKKYYNAMKKLGSK |
| | | KPQKPIPRPLNKIQGIVFDFVTQQAFDIVIMMLICLN |
| | | MVTMMVETDTQSKQMENILYWINLVFVIFFTCECV |
| | | LKMFALRHYYFTIGWNIFDFVVVILSIVGMFLADIIE |
| | | KYFVSPTLFRVIRLARIGRILRLIKGAKGIRTLLFAL |
| | | MMSLPALFNIGLLLFLVMFIFSIFGMSNFAYVKHEA |
| | | GIDDMFNFETFGNSMICLFQITTSAGWDGLLLPILN |
| | | RPPDCSLDKEHPGSGFKGDCGNPSVGIFFFVSYI |
| | | IISFLIVVNMYIAIILENFSVATEESADPLSEDDFETF |
| | | YEIWEKFDPDATQFIEYCKLADFADALEHPLRVPKP |
| | | NTIELIAMDLPMVSGDRIHCLDILFAFTKRVLGDSG |
| | | ELDILRQQMEERFVASNPSKVSYEPITTTLRRKQEE |
| | | VSAVVLQRAYRGHLARRGFICKKTTSNKLENGGTH |
| | | REKKESTPSTASLPSYDSVTKPEKEKQQRAEEGRRE |
| | | RAKRQKEVRESKC |
| 29 | huNav1.8 (alpha | MEFPIGSLETNNFRRFTPESLVEIEKQIAAKQGTKKA |
| | subunit) | REKHREQKDQEEKPRPQLDLKACNQLPKFYGELPA |
| | | ELIGEPLEDLDPFYSTHRTFMVLNKGRTISRFSATRA |
| | | LWLFSPFNLIRRTAIKVSVHSWFSLFITVTILVNCVC |
| | | MTRTDLPEKIEYVFTVIYTFEALIKILARGFCLNEFT |
| | | YLRDPWNWLDFSVITLAYVGTAIDLRGISGLRTFRV |
| | | LRALKTVSVIPGLKVIVGALIHSVKKLADVTILTIFC |
| | | LSVFALVGLQLFKGNLKNKCVKNDMAVNETTNYS |
| | | SHRKPDIYINKRGTSDPLLCGNGSDSGHCPDGYICL |
| | | KTSDNPDFNYTSFDSFAWAFLSLFRLMTQDSWERL |
| | | YQQTLRTSGKIYMIFFVLVIFLGSFYLVNLILAVVT |
| | | MAYEEQNQATTDEIEAKEKKFQEALEMLRKEQEV |
| | | LAALGIDTTSLHSHNGSPLTSKNASERRHRIKPRVS |
| | | EGSTEDNKSPRSDPYNQRRMSFLGLASGKRRASHG |
| | | SVFHFRSPGRDISLPEGVTDDGVFPGDHESHRGSLL |
| | | LGGGAGQQGPLPRSPLPQPSNPDSRHGEDEHQPPPT |
| | | SELAPGAVDVSAFDAGQKKTFLSAEYLDEPFRAQR |
| | | AMSVVSIITSVLEELEESEQKCPPCLTSLSQKYLIWD |
| | | CCPMWVKLKTILFGLVTDPFAELTITLCIVVNTIFM |
| | | AMEHHGMSPTFEAMLQIGNIVFTIFFTAEMVFKIIAF |
| | | DPYYYFQKKWNIFDCIIVTVSLLELGVAKKGSLSVL |
| | | RSFRLLRVFKLAKSWPTLNTLIKIIGNSVGALGNLTI |
| | | ILAIIVFVFALVGKQLLGENYRNNRKNISAPHEDWP |
| | | RWHMHDFFHSFLIVFRILCGEWIENMWACMEVGQ |
| | | KSICLILFLTVMVLGNLVVLNLFIALLLNSFSADNLT |
| | | APEDDGEVNNLQVALARIQVFGHRTKQALCSFFSR |
| | | SCPFPQPKAEPELVVKLPLSSSKAENHIAANTARGS |
| | | SGGLQAPRGPRDEHSDFIANPTVWVSVPIAEGESDL |
| | | DDLEDDGGEDAQSFQQEVIPKGQQEQLQQVERCG |
| | | DHLTPRSPGTGTSSEDLAPSLGETWKDESVPQVPAE |
| | | GVDDTSSSEGSTVDCLDPEEILRKIPELADDLEEPDD |
| | | CFTEGCIRHCPCCKLDTTKSPWDVGWQVRKTCYRI |
| | | VEHSWFESFIIFMILLSSGSLAFEDYYLDQKPTVKAL |
| | | LEYTDRVFTFIFVFEMLLKWVAYGFKKYFTNAWC |
| | | WLDFLIVNISLISLTAKILEYSEVAPIKALRTLRALRP |
| | | LRALSRFEGMRVVVDALVGAIPSIMNVLLVCLIFW |
| | | LIFSIMGVNLFAGKFWRCINYTDGEFSLVPLSIVNN |
| | | KSDCKIQNSTGSFFWVNVKVNFDNVAMGYLALLQ |
| | | VATFKGWMDIMYAAVDSREVNMQPKWEDNVYM |
| | | YLYFVI |
| | | FIIFGGFFTLNLFVGVIIDNFNQQKKKLGGQDIFMTE |
| | | EQKKYYNAMKKLGSKKPQKPIPRPLNKFQGFVFDI |
| | | VTRQAFDITIMVLICLNMITMMVETDDQSEEKTKIL |
| | | GKINQFFVAVFTGECVMKMFALRQYYFTNGWNVF |
| | | DFIVVVLSIASLIFSAILKSLQSYFSPTLFRVIRLARIG |
| | | RILRLIRAAKGIRTLLFALMMSLPALFNIGLLLFLVM |
| | | FIYSIFGMSSFPHVRWEAGIDDMFNFQTFANSMLCL |
| | | FQITTSAGWDGLLSPILNTGPPYCDPNLPNSNGTRG |
| | | DCGSPAVGIIFFTTYIIISFLIMVNMYIAVILENFNVA |
| | | TEESTEPLSEDDFDMFYETWEKFDPEATQFITFSALS |
| | | DFADTLSGPLRIPKPNRNILIQMDLPLVPGDKIHCLD |
| | | ILFAFTKNVLGESGELDSLKANMEEKFMATNLSKS |
| | | SYEPIATTLRWKQEDISATVIQKAYRSYVLHRSMAL |
| | | SNTPCVPRAEEEAASLPDEGFVAFTANENCVLPDKS |
| | | ETASATSFPPSYESVTRGLSDRVNMRTSSSIQNEDE |
| | | ATSMELIAPGP |
| 30 | F0103262B06 | EVQLVESGGGLVQPGGSLRLSCAGSTRTFSTYAMG |
| | (parental)-FLAG-HIS6 | WFRQAPGREREFVAHINFSGSSTRYADSVKGRFTIS |
| | | RDNAKNMGYLQMNSLKPEDTAVYYCAARWVAGP |
| | | PRYDYEYWGQGTLVTVSSAAADYKDHDGDYKDH |
| | | DIDYKDDDDKGAAHHHHHH |
| 31 | F0103262C02 | EVQLVESGGGLVQAGGSLTLSCAASGLPFGLYILG |
| | (parental)-FLAG-HIS6 | WIRRAPGKERDFVAAISRSGRDTVYANSVKGRFTIS |
| | | RDNAKNMVYLRMDNLRPEDTAAYYCAVDSVPRG |
| | | TPTITESEYAIWGQGTLVTVSSAAADYKDHDGDYK |
| | | DHDIDYKDDDDKGAAHHHHHH |
| 32 | F0103265A11 | EVQLVESGGGLVQPGGSLRLSCAASGMLFNANTQ |
| | (parental)-FLAG-HIS6 | GWYRQAPGKQRELVAFIFSGGYTNYVDSVKGRFTI |
| | | SRDNAKRTMYLQMNSLKPEDSAIYYCSLSRYLGQG |
| | | TLVTVSSAAADYKDHDGDYKDHDIDYKDDDDKG |
| | | AAHHHHHH |
| 33 | F0103265B04 | EVQLVESGGGLVQPGGSLRLSCAAPSFIFSNNYME |
| | (parental)-FLAG-HIS6 | WYRQAPGKQRDWVARITGRGNTNYLDSVKGRFTI |
| | | SRDDAKNTVYLEIDSLKPEDTAVYYCSALWYGGR |
| | | AWGKGTLVTVSSAAADYKDHDGDYKDHDIDYKD |
| | | DDDKGAAHHHHHH |
| 34 | F0103275B05 | EVQLVESGGGLVQPGGSLRLSCAASGSIFNINSMA |
| | (parental)-FLAG-HIS6 | WYRRAPGKQRELVASSTNGGSTNYADSVKGRFTIS |
| | | RDNAKRVYLQMNSLTPEDTAVYYCNALLQPSIYDI |
| | | SRTYWGQGTLVTVSSAAADYKDHDGDYKDHDID |
| | | YKDDDDKGAAHHHHHH |
| 35 | F0103362B08 | EVQLVESGGGLVQAGGSLRLSCAASVRPFSTSAMG |
| | (parental)-FLAG-HIS6 | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSSAAADYKDHDGDY |
| | | KDHDIDYKDDDDKGAAHHHHHH |
| 36 | F0103387G04 | EVQLVESGGGLAQPGGSLRLSCAASGPVFNINKMA |
| | (parental)-FLAG-HIS6 | WYRRAPGKQRELVASVTPTGSISYTDSVKGRFTISR |
| | | DGSKRWSLQMNSLTPEDTAVYYCNALLQPDSYSN |
| | | TRTYWGQGTLVTVSSAAADYKDHDGDYKDHDID |
| | | YKDDDDKGAAHHHHHH |
| 37 | F0103387G05 | EVQLVESGGGLVQAGGSLRLSCDASGRILRIGYMR |
| | (parental)-FLAG-HIS6 | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSSAAADYKDHDGDYKD |
| | | HDIDYKDDDDKGAAHHHHHH |
| 38 | F0103454D07 | EVQLVESGGGLVQPGGSLRLSCVASGGIININYIAW |
| | (parental)-FLAG-HIS6 | YRQTPGKQRDLVARISSDDTIKYGDSVKGRFAMSR |
| | | DKVKNMVHLQMNSLTTEDTGVYVCSALITPWTGD |
| | | TRTYWGRGTLVTVSSAAADYKDHDGDYKDHDID |
| | | YKDDDDKGAAHHHHHH |
| 39 | F0103464B09 | EVQLVESGGGLVQPGGSLRLSCATTSRAFIRDVFTG |
| | (parental)-FLAG-HIS6 | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSSAAADYKDHDGDYKD |
| | | HDIDYKDDDDKGAAHHHHHH |
| 40 | beta1 (beta1) subunit | MGRLLALVVGAALVSSACGGCVEVDSETEAVYGM |
| | | TFKILCISCKRRSETNAETFTEWTFRQKGTEEFVKIL |
| | | RYENEVLQLEEDERFEGRVVWNGSRGTKDLQDLSI |
| | | FITNVTYNHSGDYECHVYRLLFFENYEHNTSVVKK |
| | | IHIEVVDKANRDMASIVSEIMMYVLIVVLTIWLVAE |
| | | MIYCYKKIAAATETAAQENASEYLAITSESKENCTG |
| | | VQVAE |
| 41 | beta2 (beta2) subunit | MHRDAWLPRPAFSLTGLSLFFSLVPPGRSMEVTVP |
| | | ATLNVLNGSDARLPCTFNSCYTVNHKQFSLNWTY |
| | | QECNNCSEEMFLQFRMKIINLKLERFQDRVEFSGNP |
| | | SKYDVSVMLRNVQPEDEGIYNCYIMNPPDRHRGH |
| | | GKIHLQVLMEEPPERDSTVAVIVGASVGGFLAVVIL |
| | | VLMVVKCVRRKKEQKLSTDDLKTEEEGKTDGEGN |
| | | PDDGAK |
| 42 | beta3 (beta3) subunit | MPAFNRLFPLASLVLIYWVSVCFPVCVEVPSETEAV |
| | | QGNPMKLRCISCMKREEVEATTVVEWFYRPEGGK |
| | | DFLIYEYRNGHQEVESPFQGRLQWNGSKDLQDVSI |
| | | TVLNVTLNDSGLYTCNVSREFEFEAHRPFVKTTRLI |
| | | PLRVTEEAGEDFTSVVSEIMMYILLVFLTLWLLIEMI |
| | | YCYRKVSKAEEAAQENASDYLAIPSENKENSAVPV |
| | | EE |
| 43 | P2A viral peptide | GSGATNFSLLKQAGDVEENPGP |
| 44 | huNav1.7-beta1 | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | GTTNQIHKKRRCSSYLLSEDMLNDPNLRQRAMSRA |
| | | SILTNTVEELEESRQKCPPWWYRFAHKFLIWNCSPY |
| | | WIKFKKCIYFIVMDPFVDLAITICIVLNTLFMAMEH |
| | | HPMTEEFKNVLAIGNLVFTGIFAAEMVLKLIAMDP |
| | | YEYFQVGWNIFDSLIVTLSLVELFLADVEGLSVLRS |
| | | FRLLRVFKLAKSWPTLNMLIKIIGNSVGALGNLTLV |
| | | LAIIVFIFAVVGMQLFGKSYKECVCKINDDCTLPRW |
| | | HMNDFFHSFLIVFRVLCGEWIETMWDCMEVAGQA |
| | | MCLIVYMMVMVIGNLVVLNLFLALLLSSFSSDNLT |
| | | AIEEDPDANNLQIAVTRIKKGINYVKQTLREFILKAF |
| | | SKKPKISREIRQAEDLNTKKENYISNHTLAEMSKGH |
| | | NFLKEKDKISGFGSSVDKHLMEDSDGQSFIHNPSLT |
| | | VTVPIAPGESDLENMNAEELSSDSDSEYSKVRLNRS |
| | | SSSECSTVDNPLPGEGEEAEAEPMNSDEPEACFTDG |
| | | CVRRFSCCQVNIESGKGKIWWNIRKTCYKIVEHSW |
| | | FESFIVLMILLSSGALAFEDIYIERKKTIKIILEYADKI |
| | | FTYIFILEMLLKWIAYGYKTYFTNAWCWLDFLIVD |
| | | VSLVTLVANTLGYSDLGPIKSLRTLRALRPLRALSR |
| | | FEGMRVVVNALIGAIPSIMNVLLVCLIFWLIFSIMGV |
| | | NLFAGKFYECINTTDGSRFPASQVPNRSECFALMN |
| | | VSQNVRWKNLKVNFDNVGLGYLSLLQVATFKGW |
| | | TIIMYAAVDSVNVDKQPKYEYSLYMYIYFVVFIIFG |
| | | SFFTLNLFIGVIIDNFNQQKKKLGGQDIFMTEEQKK |
| | | YYNAMKKLGSKKPQKPIPRPGNKIQGCIFDLVTNQ |
| | | AFDISIMVLICLNMVTMMVEKEGQSQHMTEVLYWI |
| | | NVVFIILFTGECVLKLISLRHYYFTVGWNIFDFVVVI |
| | | ISIVGMFLADLIETYFVSPTLFRVIRLARIGRILRLVK |
| | | GAKGIRTLLFALMMSLPALFNIGLLLFLVMFIYAIFG |
| | | MSNFAYVKKEDGINDMFNFETFGNSMICLFQITTSA |
| | | GWDGLLAPILNSKPPDCDPKKVHPGSSVEGDCGNP |
| | | SVGIFYFVSYIIISFLVVVNMYIAVILENFSVATEEST |
| | | EPLSEDDFEMFYEVWEKFDPDATQFIEFSKLSDFAA |
| | | ALDPPLLIAKPNKVQLIAMDLPMVSGDRIHCLDILF |
| | | AFTKRVLGESGEMDSLRSQMEERFMSANPSKVSYE |
| | | PITTTLKRKQEDVSATVIQRAYRRYRLRQNVKNISS |
| | | IYIKDGDRDDDLLNKKDMAFDNVNENSSPEKTDAT |
| | | SSTTSPPSYDSVTKPDKEKYEQDRTEKEDKGKDSK |
| | | ESKKSGRGSGATNFSLLKQAGDVEENPGPMGRLLA |
| | | LVVGAALVSSACGGCVEVDSETEAVYGMTFKIL |
| | | CISCKRRSETNAETFTEWTFRQKGTEEFVKILRY |
| | | ENEVLQLEEDERFEGRVVWNGSRGTKDLQDLSI |
| | | FITNVTYNHSGDYECHVYRLLFFENYEHNTSVV |
| | | KKIHIEVVDKANRDMASIVSEIMMYVLIVVLTIW |
| | | LVAEMIYCYKKIAAATETAAQENASEYLAITSES |
| | | KENCTGVQVAE |
| 45 | muNav1.7 | MAMLPPPGPQSFVHFTKQSLALIEQRISEEKAKGHK |
| | | DEKKDDEEEGPKPSSDLEAGKQLPFIYGDIPPGMVS |
| | | EPLEDLDPYYADKKTFIVLNKGKAIFRFNATPALY |
| | | MLSPFSPLRRISIKILVHSLFSMLIMCTILTNCIFMTM |
| | | SNPPDWTKNVEYTFTGIYTFESLIKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRKDLEQNETLESIM |
| | | STAESEEELKRYFYYLEGSKDALLCGFSTDSGQCPE |
| | | GYECVTAGRNPDYGYTSFDTFGWAFLALFRLMTQ |
| | | DYWENLYQQTLRAAGKTYMIFFVVVIFLGSFYLIN |
| | | LILAVVAMAYEEQNQANIEEAKQKELEFQQMLDR |
| | | LKKEQEEAEAIAAAAAEYTSLGRSRIMGLSESSSET |
| | | SRLSSKSAKERRNRRKKKKQKLSSGEEKGDDEKLS |
| | | KSGSEESIRKKSFHLGVEGHHRAREKRLSTPNQSPL |
| | | SIRGSLFSARRSSRTSLFSFKGRGRDLGSETEFADDE |
| | | HSIFGDNESRRGSLFVPHRPRERRSSNISQASRSPPV |
| | | LPVNGKMHSAVDCNGVVSLVDGPSALMLPNGQLL |
| | | PEVIIDKATSDDSGTTNQMRKKRLSSSYFLSEDMLN |
| | | DPHLRQRAMSRASILTNTVEELEESRQKCPPWWYR |
| | | FAHTFLIWNCSPYWIKFKKFIYFIVMDPFVDLAITICI |
| | | VLNTLFMAMEHHPMTDEFKNVLAVGNLVFTGIFA |
| | | AEMVLKLIAMDPYEYFQVGWNIFDSLIVTLSLVELF |
| | | LADVEGLSVLRSFRLLRVFKLAKSWPTLNMLIKIIG |
| | | NSVGALGNLTLVLAIIVFIFAVVGMQLFGKSYKECV |
| | | CKINENCKLPRWHMNDFFHSFLIVFRVLCGEWIET |
| | | MWDCMEVAGQTMCLIVYMMVMVIGNLVVLNLFL |
| | | ALLLSSFSSDNLTAIEEDTDANNLQIAVARIKRGINY |
| | | VKQTLREFILKSFSKKPKGSKDTKRTADPNNKREN |
| | | YISNRTLAEISKDHNFLKEKDKISGFSSSLDKSFMDE |
| | | NDYQSFIHNPSLTVTVPIAPGESDLENMNTEELSSDS |
| | | DSDYSKERRNRSSSSECSTVDNPLPGEEEAEAEPIN |
| | | ADEPEACFTDGCVRRFPCCQVNIDTGKGKVWWTIR |
| | | KTCYRIVEHSWFESFIVLMILLSSGALAFEDIYIEKK |
| | | KTIKIILEYADKIFTYIFILEMLLKWVAYGYKTYFTN |
| | | AWCWLDFLIVDVSLVTLVANTLGYSDLGPIKSLRT |
| | | LRALRPLRALSRFEGMRVVVNALIGAIPSIMNVLLV |
| | | CLIFWLIFSIMGVNLFAGKFYECVNTTDGSRFSVSQ |
| | | VANRSECFALMNVSGNVRWKNLKVNFDNVGLGY |
| | | LSLLQVATFKGWMDIMYAAVDSVNVNAQPIYEYN |
| | | LYMYIYFVIFIIFGSFFTLNLFIGVIIDNFNQQKKKLG |
| | | GQDIFMTEEQKKYYNAMKKLGSKKPQKPIPRPGNK |
| | | FQGCIFDLVTNQAFDITIMVLICLNMVTMMVEKEG |
| | | QTDYMSFVLYWINVVFIILFTGECVLKLISLRHYYF |
| | | TVGWNIFDFVVVILSIVGMFLAEMIEKYFVSPTLFR |
| | | VIRLARIGRILRLIKGAKGIRTLLFALMMSLPALFNI |
| | | GLLLFLVMFIYAIFGMSNFAYVKKEAGINDMFNFE |
| | | TFGNSMICLFQITTSAGWDGLLAPILNSAPPDCDPK |
| | | KVHPGSSVEGDCGNPSVGIFYFVSYIIISFLVVVNM |
| | | YIAVILENFSVATEESTEPLSEDDFEMFYEVWEKFD |
| | | PDATQFIEFCKLSDFAAALDPPLLIAKPNKVQLIAM |
| | | DLPMVSGDRIHCLDILFAFTKRVLGESGEMDSLRSQ |
| | | MEERFMSANPSKVSYEPITTTLKRKQEDVSATIIQR |
| | | AYRRYRLRQNVKNISSIYIKDGDRDDDLPNKEDIVF |
| | | DNVNENSSPEKTDATASTISPPSYDSVTKPDQEKYE |
| | | TDKTEKEDKEKDESRK |
| 46 | F0103262B06 (parental) | EVQLVESGGGLVQPGGSLRLSCAGSTRTFSTYAMG |
| | | WFRQAPGREREFVAHINFSGSSTRYADSVKGRFTIS |
| | | RDNAKNMGYLQMNSLKPEDTAVYYCAARWVAGP |
| | | PRYDYEYWGQGTLVTVSS |
| 47 | F0103262C02 (parental) | EVQLVESGGGLVQAGGSLTLSCAASGLPFGLYILG |
| | | WIRRAPGKERDFVAAISRSGRDTVYANSVKGRFTIS |
| | | RDNAKNMVYLRMDNLRPEDTAAYYCAVDSVPRG |
| | | TPTITESEYAIWGQGTLVTVSS |
| 48 | F0103265A11 (parental) | EVQLVESGGGLVQPGGSLRLSCAASGMLFNANTQ |
| | | GWYRQAPGKQRELVAFIFSGGYTNYVDSVKGRFTI |
| | | SRDNAKRTMYLQMNSLKPEDSAIYYCSLSRYLGQG |
| | | TLVTVSS |
| 49 | F0103265B04 (parental) | EVQLVESGGGLVQPGGSLRLSCAAPSFIFSNNYME |
| | | WYRQAPGKQRDWVARITGRGNTNYLDSVKGRFTI |
| | | SRDDAKNTVYLEIDSLKPEDTAVYYCSALWYGGR |
| | | AWGKGTLVTVSS |
| 50 | F0103275B05 (parental) | EVQLVESGGGLVQPGGSLRLSCAASGSIFNINSMA |
| | | WYRRAPGKQRELVASSTNGGSTNYADSVKGRFTIS |
| | | RDNAKRVYLQMNSLTPEDTAVYYCNALLQPSIYDI |
| | | SRTYWGQGTLVTVSS |
| 51 | F0103362B08 (parental) | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 52 | F0103387G04 (parental) | EVQLVESGGGLAQPGGSLRLSCAASGPVFNINKMA |
| | | WYRRAPGKQRELVASVTPTGSISYTDSVKGRFTISR |
| | | DGSKRWSLQMNSLTPEDTAVYYCNALLQPDSYSN |
| | | TRTYWGQGTLVTVSS |
| 53 | F0103387G05 (parental) | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSS |
| 54 | F0103454D07 (parental) | EVQLVESGGGLVQPGGSLRLSCVASGGIININYIAW |
| | | YRQTPGKQRDLVARISSDDTIKYGDSVKGRFAMSR |
| | | DKVKNMVHLQMNSLTTEDTGVYVCSALITPWTGD |
| | | TRTYWGRGTLVTVSS |
| 55 | F0103464B09 (parental) | EVQLVESGGGLVQPGGSLRLSCATTSRAFIRDVFTG |
| | | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSS |
| 56 | FLAG-HIS6 peptide | AAADYKDHDGDYKDHDIDYKDDDDKGAAHHHH |
| | | HH |
| 57 | human VH3-JH | EVQLVESGGGLVQPGGSLRLSCAASGFTFSSYAMH |
| | consensus (amino acids | WVRQAPGKGLEWVSVISSSGSSTYYADSVKGRFTI |
| | Xaa99-Xaal 14 are each | SRDNSKNTLYLQMNSLRAEDTAVYYCARXXXXXX |
| | independently any | XXXXXXXXXXWGQGTLVTVSS |
| | amino acid except Cys) | |
| 58 | VHH2-consensus | QVQLVESGGGLVQAGGSLRLSCAASGSIFSINAMG |
| | | WYRQAPGKQRELVAAITSGGSTNYADSVKGRFTIS |
| | | RDNAKNTLYLQMNSLKPEDTAVYYCNA |
| 59 | F0103387G04_SO | DVQLVESGGGVVQPGGSLRLSCAASGPVFNINRMAWY |
| | | RQAPGKQRELVAYVTPTGDISYADSVKGRFTISDDGSKR |
| | | VSLQMNSLRPEDTALYYCRALLQPSSYSGTRTYWGQGT |
| | | LVTVSS |
| 60 | F0103387G05_SO | DVQLVESGGGVVQPGGSLRLSCAASGRILRIGYMRWYR |
| | | QAPGKQREFVARITGGSATGYADSVKGRFTISRDNAKN |
| | | TVYLQMNSLRPEDTALYYCEALVTASVRGGSIHSGTYW |
| | | GQGTLVTVSS |
| 61 | F0103464B09_SO | DVQLVESGGGVVQPGGSLRLSCAASHRAFIRDVFTGWY |
| | | RQAPGKQRELVARIYEGGNTQYADFAKGRFSISRDNAK |
| | | KTVYLQMNSLRAEDTALYYCLFSGTISTGREYRSGDYW |
| | | GQGTLVTVSS |
| 62 | Nav1.7 alpha epitope | FRNSLENNETLESIMNTLESEEDFRKYFYYLEGSKD |
| | | ALLCGFSTDSGQCPEGYTCV |
| 63 | Nav1.7 alpha Domain I | MAMLPPPGPQSFVHFTKQSLALIEQRIAERKSKEPK |
| | | EEKKDDDEEAPKPSSDLEAGKQLPFIYGDIPPGMVS |
| | | EPLEDLDPYYADKKTFIVLNKGKTIFRFNATPALYM |
| | | LSPFSPLRRISIKILVHSLFSMLIMCTILTNCIFMTMN |
| | | NPPDWTKNVEYTFTGIYTFESLVKILARGFCVGEFT |
| | | FLRDPWNWLDFVVIVFAYLTEFVNLGNVSALRTFR |
| | | VLRALKTISVIPGLKTIVGALIQSVKKLSDVMILTVF |
| | | CLSVFALIGLQLFMGNLKHKCFRNSLENNETLESIM |
| | | NTLESEEDFRKYFYYLEGSKDALLCGFSTDSGQCPE |
| | | GYTCVKIGRNPDYGYTSFDTFSWAFLALFRLMTQD |
| | | YWENLYQQTLRAAGKTYMIFFVVVIFLGSFYLINLI |
| | | LAVVAMAYEEQNQANIEEAKQKELEFQQMLDRLK |
| | | KEQEEAEAIAAAAAEYTSIRRSRIMGLSESSSETSKL |
| | | SSKSAKERRNRRKKKNQKKLSSGEEKGDAEKLSKS |
| | | ESEDSIRRKSFHLGVEGHRRAHEKRLSTPNQSPLSIR |
| | | GSLFSARRSSRTSLFSFKGRGRDIGSETEFADDEHSI |
| | | FGDNESRRGSLFVPHRPQERRSSNISQASRSPPMLP |
| | | VNGKMHSAVDCNGVVSLVDGRSALMLPNGQLLPE |
| | | VIIDKATSDDSGTTNQIHKKRRCSSYLLSEDMLNDP |
| | | NLRQRAMSRASILTNTVEELEESRQKCPPWWYRFA |
| | | HKFLIWNCSPYWIKFKKCIY |
| 64 | Nav1.7alpha Domain I | KHKCFRNSLENNETLESIMNTLESEEDFRKYFYYLE |
| | S5-S6 loop | GSKDALLCGFSTDSGQCPEGYTCVKIGRNPDYGYT |
| | | SFDTFSWAFLALFRLMTQDYWENLYQQTLRAAGK |
| | | TY |
| 65 | Nav1.7 alpha Exon 5N | YLTEFVNLGNVS |
| 66 | Nav1.7 alpha Exon 5A | YVTEFVDLGNVS |
| 67 | Nav1.7 alpha Exon 11S | LPNGQLLPE |
| 68 | Nav1.7 alpha Exon 11L | LPNGQLLPEVIIDKATSDDS |
| 69 | >F010301461 | EVQLVESGGGLVQPGGSLRLSCAASGSIFNINRMA |
| | F0103275B05(S33R, S50Y, | WYRRAPGKQRELVAYSTNGGDTNYADSVKGRFTI |
| | S56D, N93R) | SRDNAKRVYLQMNSLTPEDTAVYYCRALLQPSIYD |
| | | ISRTYWGQGTLVTVSS |
| 70 | >F010301635 | EVQLVESGGGVVQPGGSLRLSCAASGSIFNINSMA |
| | F0103275B05(L11V, R39Q, | WYRQAPGKQRELVASSTNGGSTNYADSVKGRFTIS |
| | T83R, V89L) | RDNAKRVYLQMNSLRPEDTALYYCNALLQPSIYDI |
| | | SRTYWGQGTLVTVSS |
| 71 | >F010301636 | EVQLVESGGGVVQPGGSLRLSCAASGSIFNINSMA |
| | F0103275B05(L11V ,R76N, | WYRRAPGKQRELVASSTNGGSTNYADSVKGRFTIS |
| | T83R, V89L) | RDNAKNVYLQMNSLRPEDTALYYCNALLQPSIYDI |
| | | SRTYWGQGTLVTVSS |
| 72 | >F010301637 | EVQLVESGGGVVQPGGSLRLSCAASGSIFNINSMA |
| | F0103275B05(L11V, T83R, | WYRRAPGKQRELVASSTNGGSTNYADSVKGRFTIS |
| | V89L) | RDNAKRVYLQMNSLRPEDTALYYCNALLQPSIYDI |
| | | SRTYWGQGTLVTVSS |
| 73 | >F010301638 | EVQLVESGGGVVQPGGSLRLSCAASGSIFNINSMA |
| | F0103275B05(L11V, R39Q, | WYRQAPGKQRELVASSTNGGSTNYADSVKGRFTIS |
| | R76N, T83R, V89L) | RDNAKNVYLQMNSLRPEDTALYYCNALLQPSIYDI |
| | | SRTYWGQGTLVTVSS |
| 74 | >F010301639 | EVQLVESGGGVVQPGGSLRLSCAASGSIFNINSMA |
| | F0103275B05(R76_V78 | WYRQAPGKQRELVASSTNGGSTNYADSVKGRFTIS |
| | insT)(L11V, R39Q, T83R, | RDNAKRTVYLQMNSLRPEDTALYYCNALLQPSIYD |
| | V89L) | ISRTYWGQGTLVTVSS |
| 75 | >F010301640 | EVQLVESGGGVVQPGGSLRLSCAASGSIFNINSMA |
| | F0103275B05(R76_V78 | WYRRAPGKQRELVASSTNGGSTNYADSVKGRFTIS |
| | insT)(L11V, T83R, V89L) | RDNAKRTVYLQMNSLRPEDTALYYCNALLQPSIYD |
| | | ISRTYWGQGTLVTVSS |
| 76 | >F010301641 | EVQLVESGGGVVQPGGSLRLSCAASGSIFNINSMA |
| | F0103275B05(R76_V78 | WYRRAPGKQRELVASSTNGGSTNYADSVKGRFTIS |
| | insT)(L11V, R76N, T83R, | RDNAKNTVYLQMNSLRPEDTALYYCNALLQPSIYD |
| | V89L) | ISRTYWGQGTLVTVSS |
| 77 | >F010301642 | EVQLVESGGGVVQPGGSLRLSCAASGSIFNINSMA |
| | F0103275B05(R76_V78 | WYRQAPGKQRELVASSTNGGSTNYADSVKGRFTIS |
| | insT)(L11V, R39Q, R76N, | RDNAKNTVYLQMNSLRPEDTALYYCNALLQPSIYD |
| | T83R, V89L) | ISRTYWGQGTLVTVSS |
| 78 | >F010301652 | EVQLVESGGGVVQPGGSLRLSCAASGSIFNINRMA |
| | F0103275B05(L11V, S33R, | WYRRAPGKQRELVAYSTNGGDTNYADSVKGRFTI |
| | S50Y, S56D, R76N, T83R, | SRDNAKNVYLQMNSLRPEDTALYYCRALLQPSIYD |
| | V89L, N93R) | ISRTYWGQGTLVTVSS |
| 79 | >F010301653 | EVQLVESGGGVVQPGGSLRLSCAASGSIFNINRMA |
| | F0103275B05(L11V, S33R, | WYRRAPGKQRELVAYSTNGGDTNYADSVKGRFTI |
| | S50Y, S56D, T83R, V89L, | SRDNAKRVYLQMNSLRPEDTALYYCRALLQPSIYD |
| | N93R) | ISRTYWGQGTLVTVSS |
| 80 | >F010301654 | EVQLVESGGGVVQPGGSLRLSCAASGSIFNINRMA |
| | F0103275B05(L11V, S33R, | WYRQAPGKQRELVAYSTNGGDTNYADSVKGRFTI |
| | R39Q, S50Y, S56D, T83R, | SRDNAKRVYLQMNSLRPEDTALYYCRALLQPSIYD |
| | V89L, N93R) | ISRTYWGQGTLVTVSS |
| 81 | >F010301655 | EVQLVESGGGVVQPGGSLRLSCAASGSIFNINRMA |
| | F0103275B05(L11V, S33R, | WYRQAPGKQRELVAYSTNGGDTNYADSVKGRFTI |
| | R39Q, S50Y, S56D, R76N, | SRDNAKNVYLQMNSLRPEDTALYYCRALLQPSIYD |
| | T83R, V89L, N93R) | ISRTYWGQGTLVTVSS |
| 82 | >F010301556 | EVQLVESGGGLVQAGGSLRLSCAASGRILRIGYMR |
| | F0103387G05(D23A, D53G, | WHRQGAGKQREFVARITGGSATGYADSVKGRFTIS |
| | D54G, D58G) | RDNAKNTVYLQMNNLNPEDTAVYYCEALVTASVR |
| | | GGSIHSGTYWGRGTLVTVSS |
| 83 | >F010301563 | EVQLVESGGGLVQAGGSLRLSCAASGRILRIGYMR |
| | F0103387G05(D23A, D58G) | WHRQGAGKQREFVARITDDSATGYADSVKGRFTIS |
| | | RDNAKNTVYLQMNNLNPEDTAVYYCEALVTASVR |
| | | GGSIHSGTYWGRGTLVTVSS |
| 84 | >F010301849 | EVQLVESGGGVVQPGGSLRLSCAASGRILRIGYMR |
| | F0103387G05(L11V, A14P, | WHRQAPGKQREFVARITDDSATGYADSVKGRFTIS |
| | D23A, G40A, A41P, D58G, | RDNAKNTVYLQMNSLRPEDTALYYCEALVTASVR |
| | N82bS, N83R, V89L, | GGSIHSGTYWGQGTLVTVSS |
| | R105Q) | |
| 85 | >F010301850 | EVQLVESGGGVVQPGGSLRLSCAASGRILRIGYMR |
| | F0103387G05(L11V, A14P, | WYRQAPGKQREFVARITDDSATGYADSVKGRFTIS |
| | D23A, H37Y, G40A, | RDNAKNTVYLQMNSLRPEDTALYYCEALVTASVR |
| | A41P, D58G, N82bS, N83R, | GGSIHSGTYWGQGTLVTVSS |
| | V89L, R105Q) | |
| 86 | >F010301643 | EVQLVESGGGVVQPGGSLRLSCAASGRILRIGYMR |
| | F0103387G05(L11V, A14P, | WHRQGAGKQREFVARITDDSATDYADSVKGRFTIS |
| | D23A, N82bS, N83R, | RDNAKNTVYLQMNSLRPEDTALYYCEALVTASVR |
| | V89L, R105Q) | GGSIHSGTYWGQGTLVTVSS |
| 87 | >F010301644 | EVQLVESGGGVVQPGGSLRLSCDASGRILRIGYMR |
| | F0103387G05(L11V, A14P, | WYRQGAGKQREFVARITDDSATDYADSVKGRFTIS |
| | H37Y, N82bS, N83R, | RDNAKNTVYLQMNSLRPEDTALYYCEALVTASVR |
| | V89L, R105Q) | GGSIHSGTYWGQGTLVTVSS |
| 88 | >F010301645 | EVQLVESGGGVVQPGGSLRLSCDASGRILRIGYMR |
| | F0103387G05(L11V, A14P, | WHRQAAGKQREFVARITDDSATDYADSVKGRFTIS |
| | G40A, N82bS, N83R, | RDNAKNTVYLQMNSLRPEDTALYYCEALVTASVR |
| | V89L, R105Q) | GGSIHSGTYWGQGTLVTVSS |
| 89 | >F010301646 | EVQLVESGGGVVQPGGSLRLSCDASGRILRIGYMR |
| | F0103387G05(L11V, A14P, | WHRQGPGKQREFVARITDDSATDYADSVKGRFTIS |
| | A41P, N82bS, N83R, | RDNAKNTVYLQMNSLRPEDTALYYCEALVTASVR |
| | V89L, R105Q) | GGSIHSGTYWGQGTLVTVSS |
| 90 | >F010301647 | EVQLVESGGGVVQPGGSLRLSCDASGRILRIGYMR |
| | F0103387G05(L11V, A14P, | WHRQGAGKQRELVARITDDSATDYADSVKGRFTIS |
| | F47L, N82bS, N83R, | RDNAKNTVYLQMNSLRPEDTALYYCEALVTASVR |
| | V89L, R105Q) | GGSIHSGTYWGQGTLVTVSS |
| 91 | >F010301648 | EVQLVESGGGVVQPGGSLRLSCDASGRILRIGYMR |
| | F0103387G05(L11V, A14P, | WHRQGAGKQREFVARITDDSATDYADSVKGRFTIS |
| | N82bS, N83R, V89L, | RDNAKNTVYLQMNSLRPEDTALYYCNALVTASVR |
| | E93N, R105Q) | GGSIHSGTYWGQGTLVTVSS |
| 92 | >F010301649 | EVQLVESGGGVVQPGGSLRLSCDASGRILRIGYMR |
| | F0103387G05(L11V, A14P, | WHRQGAGKQREFVARITDDSATDYADSVKGRFTIS |
| | N82bS, N83R, V89L, | RDNAKNTVYLQMNSLRPEDTALYYCEALVTASVR |
| | R105Q) | GGSIHSGTYWGQGTLVTVSS |
| 93 | >F010302307 | EVQLVESGGGVVQPGGSLRLSCAASGRILRIGYMR |
| | F0103387G05(L11V, A14P, | WYRQAPGKQREFVARITDDSATGYADSVKGRFTIS |
| | D23A, H37Y, G40A, | RDAAKNTVYLQMNSLRPEDTALYYCEALVTASVR |
| | A41P, D58G, N73A, N82bS, | GGSIHSGTYWGQGTLVTVSS |
| | N83R, V89L, R105Q) | |
| 94 | >F010302308 | EVQLVESGGGVVQPGGSLRLSCAASGRILRIGYMR |
| | F0103387G05(L11V, A14P, | WYRQAPGKQREFVARITDDSATGYADSVKGRFTIS |
| | D23A, H37Y, G40A, | RDYAKNTVYLQMNSLRPEDTALYYCEALVTASVR |
| | A41P, D58G, N73Y, N82bS, | GGSIHSGTYWGQGTLVTVSS |
| | N83R, V89L, R105Q) | |
| 95 | >F010302309 | EVQLVESGGGVVQPGGSLRLSCAASGRILRIGYMR |
| | F0103387G05(L11V, A14P, | WYRQAPGKQREFVARITDDSATGYADSVKGRFTIS |
| | D23A, H37Y, G40A, | RDQAKNTVYLQMNSLRPEDTALYYCEALVTASVR |
| | A41P, D58G, N73Q, N82bS, | GGSIHSGTYWGQGTLVTVSS |
| | N83R, V89L, R105Q) | |
| 96 | >F010302391 | EVQLVESGGGVVQPGGSLRLSCAASGRILRIGYMR |
| | F0103387G05(L11V, A14P, | WYRQAPGKQREFVARITGGSATGYADSVKGRFTIS |
| | D23A, H37Y, G40A, | RDNAKNTVYLQMNSLRPEDTALYYCEALVTASVR |
| | A41P,D53G, D54G, D58G, | GGSIHSGTYWGQGTLVTVSS |
| | N82bS, N83R, V89L, | |
| | R105Q) | |
| 97 | >F010302392 | EVQLVESGGGVVQPGGSLRLSCAASGRILRIGYMR |
| | F0103387G05(L11V, A14P, | WYRQAPGKQREFVARITGGSATGYADSVKGRFTIS |
| | D23A, H37Y, G40A, | RDQAKNTVYLQMNSLRPEDTALYYCEALVTASVR |
| | A41P, D53G, D54G, D58G, | GGSIHSGTYWGQGTLVTVSS |
| | N73Q, N82bS, N83R, | |
| | V89L, R105Q) | |
| 98 | >F010301868 | EVQLVESGGGVVQPGGSLRLSCATTSRAFIRDVFTG |
| | F0103464B09(L11V, S68T, | WYRRVPGKERELVARIYNGGNTNYADFAKGRFTIS |
| | T79Y, R81Q, S82aN, | RDNAKKMVYLQMNSLRPEDTALYYCLFSGTINTG |
| | N82bS, K83R, G88A, | REYRSGDYWGQGTLVTVSS |
| | V89L) | |
| 99 | >F010301869 | EVQLVESGGGVVQPGGSLRLSCATTSRAFIRDVFTG |
| | F0103464B09(L11V, S68T, | WYRRVPGKERELVARIYNGGNTNY |
| | M77T, T79Y, R81Q, | ADFAKGRFTISRDNAKKTVYLQMNSLRPEDTALYY |
| | S82aN, N82bS, K83R, G88A, | CLFSGTINTGREYRSGDYWGQGTLVTVSS |
| | V89L) | |
| 100 | >F010301870 | EVQLVESGGGVVQPGGSLRLSCATTSRAFIRDVFTG |
| | F0103464B09(L11V, S68T, | WYRRVPGKERELVARIYNGGNTNYADFAKGRFTIS |
| | T79Y, R81Q, S82aN, | RDNAKKMVYLQMNSLRPEDTALYYCNFSGTINTG |
| | N82bS, K83R, G88A, V89L, | REYRSGDYWGQGTLVTVSS |
| | L93N) | |
| 101 | >F010301871 | EVQLVESGGGVVQPGGSLRLSCAATSRAFIRDVFT |
| | F0103464B09(L11V, T24A, | GWYRRVPGKERELVARIYNGGNTNYADFAKGRFT |
| | S68T, T79Y, R81Q, | ISRDNAKKMVYLQMNSLRPEDTALYYCLFSGTINT |
| | S82aN, N82bS, K83R, | GREYRSGDYWGQGTLVTVSS |
| | AG88, V89L) | |
| 102 | >F010301872 | EVQLVESGGGVVQPGGSLRLSCATSSRAFIRDVFTG |
| | F0103464B09(L11V, T25S, | WYRRVPGKERELVARIYNGGNTNYADFAKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 103 | >F010301873 | EVQLVESGGGVVQPGGSLRLSCATTSRAFIRDVFTG |
| | F0103464B09(L11V, R39Q, | WYRQVPGKERELVARIYNGGNTNYADFAKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 104 | >F010301874 | EVQLVESGGGVVQPGGSLRLSCATTSRAFIRDVFTG |
| | F0103464B09(L11V, V40A, | WYRRAPGKERELVARIYNGGNTNYADFAKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 105 | >F010301875 | EVQLVESGGGVVQPGGSLRLSCATTSRAFIRDVFTG |
| | F0103464B09(L11V, F62S, | WYRRVPGKERELVARIYNGGNTNYADSAKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 106 | >F010301876 | EVQLVESGGGVVQPGGSLRLSCATTSRAFIRDVFTG |
| | F0103464B09(L11V, A63V, | WYRRVPGKERELVARIYNGGNTNYADFVKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 107 | >F010301877 | EVQLVESGGGVVQPGGSLRLSCATTSRAFIRDVFTG |
| | F0103464B09(L11V, S68T, | WYRRVPGKERELVARIYNGGNTNYADFAKGRFTIS |
| | K76N, T79Y, R81Q, | RDNAKNMVYLQMNSLRPEDTALYYCLFSGTINTG |
| | S82aN, N82bS, K83R, | REYRSGDYWGQGTLVTVSS |
| | G88A, V89L) | |
| 108 | >F010301892 | EVQLVESGGGLVQPGGSLRLSCATTSRAFIRDLFTG |
| | F0103464B09(V33L) | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSS |
| 109 | >F010301893 | EVQLVESGGGVVQPGGSLRLSCATTSRAFIRDVFTG |
| | F0103464B09(L11V, E44Q, | WYRRVPGKQRELVARIYNGGNTNYADFAKGRFTIS |
| | S68T, T79Y, R81Q, | RDNAKKMVYLQMNSLRPEDTALYYCLFSGTINTG |
| | S82aN, N82bS, K83R, | REYRSGDYWGQGTLVTVSS |
| | G88A, V89L) | |
| 110 | >F010301932 | EVQLVESGGGVVQPGGSLRLSCATTSRAFIRDVFTG |
| | F0103464B09(L11V, K83R, | WYRRVPGKERELVARIYNGGNTNYADFAKGRFSIS |
| | V89L) | RDNAKKMVTLRMSNLRPEDTGLYYCLFSGTINTGR |
| | | EYRSGDYWGQGTLVTVSS |
| 111 | >F010301933 | EVQLVESGGGVVQPGGSLRLSCATTSRAFIRDVFTG |
| | F0103464B09(L11V, S68T, | WYRRVPGKERELVARIYNGGNTNYADFAKGRFTIS |
| | K83R, V89L) | RDNAKKMVTLRMSNLRPEDTGLYYCLFSGTINTGR |
| | | EYRSGDYWGQGTLVTVSS |
| 112 | >F010301934 | EVQLVESGGGVVQPGGSLRLSCATTSRAFIRDVFTG |
| | F0103464B09(L11V, M77T, | WYRRVPGKERELVARIYNGGNTNYADFAKGRFSIS |
| | K83R, V89L) | RDNAKKTVTLRMSNLRPEDTGLYYCLFSGTINTGR |
| | | EYRSGDYWGQGTLVTVSS |
| 113 | >F010301935 | EVQLVESGGGVVQPGGSLRLSCATTSRAFIRDVFTG |
| | F0103464B09(L11V, T79Y, | WYRRVPGKERELVARIYNGGNTNYADFAKGRFSIS |
| | K83R, V89L) | RDNAKKMVYLRMSNLRPEDTGLYYCLFSGTINTG |
| | | REYRSGDYWGQGTLVTVSS |
| 114 | >F010301936 | EVQLVESGGGVVQPGGSLRLSCATTSRAFIRDVFTG |
| | F0103464B09(L11V, R81Q, | WYRRVPGKERELVARIYNGGNTNYADFAKGRFSIS |
| | K83R, V89L) | RDNAKKMVTLQMSNLRPEDTGLYYCLFSGTINTGR |
| | | EYRSGDYWGQGTLVTVSS |
| 115 | >F010301937 | EVQLVESGGGVVQPGGSLRLSCATTSRAFIRDVFTG |
| | F0103464B09(L11V, S82aN, | WYRRVPGKERELVARIYNGGNTNYADFAKGRFSIS |
| | K83R, V89L) | RDNAKKMVTLRMNNLRPEDTGLYYCLFSGTINTG |
| | | REYRSGDYWGQGTLVTVSS |
| 116 | >F010301938 | EVQLVESGGGVVQPGGSLRLSCATTSRAFIRDVFTG |
| | F0103464B09(L11V, N82bS, | WYRRVPGKERELVARIYNGGNTNYADFAKGRFSIS |
| | K83R, V89L) | RDNAKKMVTLRMSSLRPEDTGLYYCLFSGTINTGR |
| | | EYRSGDYWGQGTLVTVSS |
| 117 | >F010301939 | EVQLVESGGGVVQPGGSLRLSCATTSRAFIRDVFTG |
| | F0103464B09(L11V, K83R, | WYRRVPGKERELVARIYNGGNTNYADFAKGRFSIS |
| | G88A, V89L) | RDNAKKMVTLRMSNLRPEDTALYYCLFSGTINTGR |
| | | EYRSGDYWGQGTLVTVSS |
| 118 | >F010302333 | EVQLVESGGGVVQPGGSLRLSCAASSRQFIRDVFT |
| | F0103464B09(L11V, T24A, | GWYRRAPGKQRELVARIYNEGNTNYADSAKGRFT |
| | T25S, A28Q, V40A, | ISRDNAKKTVYLQMNSLRPEDTALYYCLFSGTISTG |
| | E44Q, G54E, F62S, S68T, | REYRSGDYWGQGTLVTVSS |
| | M77T, T79Y, R81Q, S82aN, | |
| | N82bS, K83R, G88A, | |
| | V89L, N99S) | |
| 119 | >F010302334 | EVQLVESGGGVVQPGGSLRLSCAASSRQFIRDVFT |
| | F0103464B09(L11V, T24A, | GWYRRAPGKQRELVARIYNEGNTQYADSAKGRFT |
| | T25S, A28Q, V40A, | ISRDNAKKTVYLQMNSLRPEDTALYYCLFSGTISTG |
| | E44Q, G54E, N58Q, F62S, | REYRSGDYWGQGTLVTVSS |
| | S68T, M77T, T79Y, R81Q, | |
| | S82aN, N82bS, K83R, | |
| | G88A, V89L, N99S) | |
| 120 | >F010302335 | EVQLVESGGGVVQPGGSLRLSCAASSRQFIRDVFT |
| | F0103464B09(L11V, T24A, | GWYRRAPGKQRELVARIYESGNTQYADSAKGRFTI |
| | T25S, A28Q, V40A, | SRDNAKKTVYLQMNSLRPEDTALYYCLFSGTISTG |
| | E44Q, N53E, G54S, N58Q, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 121 | >F010302336 | EVQLVESGGGVVQPGGSLRLSCAASHRQFIRDVFT |
| | F0103464B09(L11V, T24A, | GWYRRAPGKQRELVARIYNEGNTQYADSAKGRFT |
| | T25S, S26H, A28Q, V40A, | ISRDNAKKTVYLQMNSLRPEDTALYYCLFSGTISTG |
| | E44Q, G54E, N58Q, | REYRSGDYWGQGTLVTVSS |
| | F62S, S68T, M77T, T79Y | |
| | R81Q, S82aN, N82bS, | |
| | K83R, G88A, V89L, | |
| | N99S) | |
| 122 | >F010302337 | EVQLVESGGGVVQPGGSLRLSCAASHRQFIRDVFT |
| | F0103464B09(L11V, T24A, | GWYRRAPGKQRELVARIYEGGNTQYADSAKGRFT |
| | T25S, S26H, A28Q, | ISRDNAKKTVYLQMNSLRPEDTALYYCLFSGTISTG |
| | V40A, E44Q, N53E, N58Q, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 123 | >F010302338 | EVQLVESGGGVVQPGGSLRLSCAASHRAFIRDVFT |
| | F0103464B09(L11V, T24A, | GWYRRAPGKQRELVARIYESGNTQYADSAKGRFTI |
| | T25S, S26H, V40A, | SRDNAKKTVYLQMNSLRPEDTALYYCLFSGTISTG |
| | E44Q, N53E, G54S, N58Q, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 124 | >F010302339 | EVQLVESGGGVVQPGGSLRLSCAASHRAFIRDVFT |
| | F0103464B09(L11V, T24A, | GWYRRAPGKQRELVARIYEGGNTQYADSAKGRFT |
| | T25S, S26H, V40A, | ISRDNAKKTVYLQMNSLRPEDTALYYCLFSGTISTG |
| | E44Q, N53E, N58Q, F62S, | REYRSGDYWGQGTLVTVSS |
| | S68T, M77T, T79Y, R81Q, | |
| | S82aN, N82bS, K83R, | |
| | G88A, V89L, N99S) | |
| 125 | >F010302340 | EVQLVESGGGVVQPGGSLRLSCAASHRAFIRDLFT |
| | F0103464B09(L11V, T24A, | GWYRRAPGKQRELVARIYESGNTNYADSAKGRFTI |
| | T25S, S26H, V33L, | SRDNAKKTVYLQMNSLRPEDTALYYCLFSGTISTG |
| | V40A, E44Q, N53E, G54S, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 126 | >F010302341 | EVQLVESGGGVVQPGGSLRLSCAASSRQFIRDVFT |
| | F0103464B09(L11V, T24A, | GWYRQAPGKQRELVARIYNEGNTNYADSAKGRFT |
| | T25S, A28Q, R39Q, | ISRDNAKKTVYLQMNSLRPEDTALYYCLFSGTISTG |
| | V40A, E44Q, G54E, F62S, | REYRSGDYWGQGTLVTVSS |
| | S68T, M77T, T79Y, R81Q, | |
| | S82aN, N82bS, K83R, | |
| | G88A, V89L, N99S) | |
| 127 | >F010302342 | EVQLVESGGGVVQPGGSLRLSCAASSRQFIRDVFT |
| | F0103464B09(L11V, T24A, | GWYRQAPGKQRELVARIYNEGNTQYADSAKGRFT |
| | T25S, A28Q, R39Q, | ISRDNAKKTVYLQMNSLRPEDTALYYCLFSGTISTG |
| | V40A, E44Q, G54E, N58Q, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 128 | >F010302343 | EVQLVESGGGVVQPGGSLRLSCAASSRQFIRDVFT |
| | F0103464B09(L11V, T24A, | GWYRQAPGKQRELVARIYESGNTQYADSAKGRFTI |
| | T25S, A28Q, R39Q, V40A, | SRDNAKKTVYLQMNSLRPEDTALYYCLFSGTISTG |
| | E44Q, N53E, G54S, | REYRSGDYWGQGTLVTVSS |
| | N58Q, F62S, S68T, M77T, | |
| | T79Y, R81Q, S82aN, | |
| | N82bS, K83R, G88A, | |
| | V89L, N99S) | |
| 129 | >F010302344 | EVQLVESGGGVVQPGGSLRLSCAASHRQFIRDVFT |
| | F0103464B09(L11V, T24A, | GWYRQAPGKQRELVARIYNEGNTQYADSAKGRFT |
| | T25S, S26H, A28Q, | ISRDNAKKTVYLQMNSLRPEDTALYYCLFSGTISTG |
| | R39Q, V40A, E44Q, G54E, | REYRSGDYWGQGTLVTVSS |
| | N58Q, F62S, S68T, M77T, | |
| | T79Y, R81Q, S82aN, | |
| | N82bS, K83R, G88A, | |
| | V89L, N99S) | |
| 130 | >F010302345 | EVQLVESGGGVVQPGGSLRLSCAASHRQFIRDVFT |
| | F0103464B09(L11V, T24A, | GWYRQAPGKQRELVARIYEGGNTQYADSAKGRFT |
| | T25S, S26H, A28Q, | ISRDNAKKTVYLQMNSLRPEDTALYYCLFSGTISTG |
| | R39Q, V40A, E44Q, N53E, | REYRSGDYWGQGTLVTVSS |
| | N58Q, F62S, S68T, M77T, | |
| | T79Y, R81Q, S82aN, | |
| | N82bS, K83R, G88A, | |
| | V89L, N99S) | |
| 131 | >F010302346 | EVQLVESGGGVVQPGGSLRLSCAASHRAFIRDVFT |
| | F0103464B09(L11V, T24A, | GWYRQAPGKQRELVARIYESGNTQYADSAKGRFTI |
| | T25S, S26H, R39Q, | SRDNAKKTVYLQMNSLRPEDTALYYCLFSGTISTG |
| | V40A, E44Q, N53E, G54S, | REYRSGDYWGQGTLVTVSS |
| | N58Q, F62S, S68T, M77T, | |
| | T79Y, R81Q, S82aN, | |
| | N82bS, K83R, G88A, | |
| | V89L, N99S) | |
| 132 | >F010302347 | EVQLVESGGGVVQPGGSLRLSCAASHRAFIRDVFT |
| | F0103464B09(L11V, T24A, | GWYRQAPGKQRELVARIYEGGNTQYADSAKGRFT |
| | T25S, S26H, R39Q, | ISRDNAKKTVYLQMNSLRPEDTALYYCLFSGTISTG |
| | V40A, E44Q, N53E, N58Q, | REYRSGDYWGQGTLVTVSS |
| | F62S, S68T, M77T, T79Y, | |
| | R81Q, S82aN, N82bS, | |
| | K83R, G88A, V89L, | |
| | N99S) | |
| 133 | >F010302348 | EVQLVESGGGVVQPGGSLRLSCAASHRAFIRDLFT |
| | F0103464B09(L11V, T24A, | GWYRQAPGKQRELVARIYESGNTNYADSAKGRFTI |
| | T25S, S26H, V33L, | SRDNAKKTVYLQMNSLRPEDTALYYCLFSGTISTG |
| | R39Q, V40A, E44Q, N53E, | REYRSGDYWGQGTLVTVSS |
| | G54S,F62S, S68T, M77T, | |
| | T79Y, R81Q, S82aN, | |
| | N82bS, K83R, G88A, | |
| | V89L, N99S) | |
| 134 | >F010302349 | EVQLVESGGGVVQPGGSLRLSCAASSRQFIRDVFT |
| | F0103464B09(L11V, T24A, | GWYRRAPGKQRELVARIYNEGNTNYADSVKGRFT |
| | T25S, A28Q, V40A, | ISRDNAKKTVYLQMNSLRPEDTALYYCLFSGTISTG |
| | E44Q, G54E, F62S, A63V, | REYRSGDYWGQGTLVTVSS |
| | S68T, M77T, T79Y, R81Q, | |
| | S82aN, N82bS, K83R, | |
| | G88A, V89L, N99S) | |
| 135 | >F010302350 | EVQLVESGGGVVQPGGSLRLSCAASSRQFIRDVFT |
| | F0103464B09(L11V, T24A, | GWYRRAPGKQRELVARIYNEGNTQYADSVKGRFT |
| | T25S, A28Q, V40A, | ISRDNAKKTVYLQMNSLRPEDTALYYCLFSGTISTG |
| | E44Q, G54E, N58Q, F62S, | REYRSGDYWGQGTLVTVSS |
| | A63V, S68T, M77T, T79Y, | |
| | R81Q, S82aN, N82bS, | |
| | K83R, G88A, V89L, | |
| | N99S) | |
| 136 | >F010302351 | EVQLVESGGGVVQPGGSLRLSCAASSRQFIRDVFT |
| | F0103464B09(L11V, T24A, | GWYRRAPGKQRELVARIYESGNTQYADSVKGRFTI |
| | T25S, A28Q, V40A, | SRDNAKKTVYLQMNSLRPEDTALYYCLFSGTISTG |
| | E44Q, N53E, G54S, N58Q, | REYRSGDYWGQGTLVTVSS |
| | F62S, A63V, S68T, M77T, | |
| | T79Y, R81Q, S82aN, N82bS, | |
| | K83R, G88A, | |
| | V89L, N99S) | |
| 137 | >F010302352 | EVQLVESGGGVVQPGGSLRLSCAASHRQFIRDVFT |
| | F0103464B09(L11V, T24A, | GWYRRAPGKQRELVARIYNEGNTQYADSVKGRFT |
| | T25S, S26H, A28Q, | ISRDNAKKTVYLQMNSLRPEDTALYYCLFSGTISTG |
| | V40A, E44Q, G54E, N58Q, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 138 | >F010302353 | EVQLVESGGGVVQPGGSLRLSCAASHRQFIRDVFT |
| | F0103464B09(L11V, T24A, | GWYRRAPGKQRELVARIYEGGNTQYADSVKGRFT |
| | T25S, S26H, A28Q, | ISRDNAKKTVYLQMNSLRPEDTALYYCLFSGTISTG |
| | V40A, E44Q, N53E, N58Q, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 139 | >F010302354 | EVQLVESGGGVVQPGGSLRLSCAASHRAFIRDVFT |
| | F0103464B09(L11V, T24A, | GWYRRAPGKQRELVARIYESGNTQYADSVKGRFTI |
| | T25S, S26H, V40A, | SRDNAKKTVYLQMNSLRPEDTALYYCLFSGTISTG |
| | E44Q, N53E, G54S, N58Q, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 140 | >F010302355 | EVQLVESGGGVVQPGGSLRLSCAASHRAFIRDVFT |
| | F0103464B09(L11V, T24A, | GWYRRAPGKQRELVARIYEGGNTQYADSVKGRFT |
| | T25S, S26H, V40A, | ISRDNAKKTVYLQMNSLRPEDTALYYCLFSGTISTG |
| | E44Q, N53E, N58Q, F62S, | REYRSGDYWGQGTLVTVSS |
| | A63V, S68T, M77T, T79Y, | |
| | R81Q, S82aN, N82bS, | |
| | K83R, G88A, V89L, | |
| | N99S) | |
| 141 | >F010302356 | EVQLVESGGGVVQPGGSLRLSCAASHRAFIRDLFT |
| | F0103464B09(L11V, T24A, | GWYRRAPGKQRELVARIYESGNTNYADSVKGRFTI |
| | T25S, S26H, V33L, | SRDNAKKTVYLQMNSLRPEDTALYYCLFSGTISTG |
| | V40A, E44Q, N53E, G54S, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 142 | >F010302357 | EVQLVESGGGVVQPGGSLRLSCAASSRQFIRDVFT |
| | F0103464B09(L11V, T24A, | GWYRQAPGKQRELVARIYNEGNTNYADSVKGRFT |
| | T25S, A28Q, R39Q, | ISRDNAKKTVYLQMNSLRPEDTALYYCLFSGTISTG |
| | V40A, E44Q, G54E, F62S, | REYRSGDYWGQGTLVTVSS |
| | A63V, S68T, M77T, T79Y, | |
| | R81Q, S82aN, N82bS, | |
| | K83R, G88A, V89L, | |
| | N99S) | |
| 143 | >F010302358 | EVQLVESGGGVVQPGGSLRLSCAASSRQFIRDVFT |
| | F0103464B09(L11V, T24A, | GWYRQAPGKQRELVARIYNEGNTQYADSVKGRFT |
| | T25S, A28Q, R39Q, | ISRDNAKKTVYLQMNSLRPEDTALYYCLFSGTISTG |
| | V40A, E44Q, G54E, N58Q, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 144 | >F010302359 | EVQLVESGGGVVQPGGSLRLSCAASSRQFIRDVFT |
| | F0103464B09(L11V, T24A, | GWYRQAPGKQRELVARIYESGNTQYADSVKGRFTI |
| | T25S, A28Q, R39Q, | SRDNAKKTVYLQMNSLRPEDTALYYCLFSGTISTG |
| | V40A, E44Q, N53E, G54S, | REYRSGDYWGQGTLVTVSS |
| | N58Q, F62S, A63V, S68T, | |
| | M77T, T79Y, R81Q, S82aN, | |
| | N82bS, K83R, | |
| | G88A, V89L, N99S) | |
| 145 | >F010302360 | EVQLVESGGGVVQPGGSLRLSCAASHRQFIRDVFT |
| | F0103464B09(L11V, T24A, | GWYRQAPGKQRELVARIYNEGNTQYADSVKGRFT |
| | T25S, S26H, A28Q, | ISRDNAKKTVYLQMNSLRPEDTALYYCLFSGTISTG |
| | R39Q, V40A, E44Q, G54E, | REYRSGDYWGQGTLVTVSS |
| | N58Q, F62S, A63V, S68T, | |
| | M77T, T79Y, R81Q, S82aN, | |
| | N82bS, K83R, | |
| | G88A, V89L, N99S) | |
| 146 | >F010302361 | EVQLVESGGGVVQPGGSLRLSCAASHRQFIRDVFT |
| | F0103464B09(L11V, T24A, | GWYRQAPGKQRELVARIYEGGNTQYADSVKGRFT |
| | T25S, S26H, A28Q, | ISRDNAKKTVYLQMNSLRPEDTALYYCLFSGTISTG |
| | R39Q, V40A, E44Q, N53E, | REYRSGDYWGQGTLVTVSS |
| | N58Q, F62S, A63V, S68T, | |
| | M77T, T79Y, R81Q, S82aN, | |
| | N82bS, K83R, | |
| | G88A, V89L, N99S) | |
| 147 | >F010302362 | EVQLVESGGGVVQPGGSLRLSCAASHRAFIRDVFT |
| | F0103464B09(L11V, T24A, | GWYRQAPGKQRELVARIYESGNTQYADSVKGRFTI |
| | T25S, S26H, R39Q, | SRDNAKKTVYLQMNSLRPEDTALYYCLFSGTISTG |
| | V40A, E44Q, N53E, G54S, | REYRSGDYWGQGTLVTVSS |
| | N58Q, F62S, A63V, S68T, | |
| | M77T, T79Y, R81Q, | |
| | S82aN, N82bS, K83R, | |
| | G88A, V89L, N99S) | |
| 148 | >F010302363 | EVQLVESGGGVVQPGGSLRLSCAASHRAFIRDVFT |
| | F0103464B09(L11V, T24A, | GWYRQAPGKQRELVARIYEGGNTQYADSVKGRFT |
| | T25S, S26H, R39Q, | ISRDNAKKTVYLQMNSLRPEDTALYYCLFSGTISTG |
| | V40A, E44Q, N53E, N58Q, | REYRSGDYWGQGTLVTVSS |
| | F62S, A63V, S68T, M77T, | |
| | T79Y, R81Q, S82aN, | |
| | N82bS, K83R, G88A, | |
| | V89L, N99S) | |
| 149 | >F010302364 | EVQLVESGGGVVQPGGSLRLSCAASHRAFIRDLFT |
| | F0103464B09(L11V, T24A, | GWYRQAPGKQRELVARIYESGNTNYADSVKGRFTI |
| | T25S, S26H, V33L, | SRDNAKKTVYLQMNSLRPEDTALYYCLFSGTISTG |
| | R39Q, V40A, E44Q, N53E, | REYRSGDYWGQGTLVTVSS |
| | G54S, F62S, A63V, S68T, | |
| | M77T, T79Y, R81Q, | |
| | S82aN, N82bS, K83R, | |
| | G88A, V89L, N99S) | |
| 150 | >F010302365 | EVQLVESGGGVVQPGGSLRLSCAASSRAFIRDVFT |
| | F0103464B09(L11V, T24A, | GWYRRAPGKQRELVARIYNGGNTNYADSAKGRFT |
| | T25S, V40A, E44Q, | ISRDNAKKTVYLQMNSLRPEDTALYYCLFSGTISTG |
| | F62S, S68T, M77T, T79Y, | REYRSGDYWGQGTLVTVSS |
| | R81Q, S82aN, N82bS, | |
| | K83R, G88A, V89L, N99S) | |
| 151 | >F010302366 | EVQLVESGGGVVQPGGSLRLSCAASSRAFIRDVFT |
| | F0103464B09(L11V, T24A, | GWYRQAPGKQRELVARIYNGGNTNYADSAKGRFT |
| | T25S, R39Q, V40A, | ISRDNAKKTVYLQMNSLRPEDTALYYCLFSGTISTG |
| | E44Q, F62S, S68T, M77T, | REYRSGDYWGQGTLVTVSS |
| | T79Y, R81Q, S82aN, | |
| | N82bS, K83R, G88A, V89L, | |
| | N99S) | |
| 152 | >F010302367 | EVQLVESGGGVVQPGGSLRLSCAASSRAFIRDVFT |
| | F0103464B09(L11V, T24A, | GWYRRAPGKQRELVARIYNGGNTNYADSVKGRFT |
| | T25S, V40A, E44Q, | ISRDNAKKTVYLQMNSLRPEDTALYYCLFSGTISTG |
| | F62S, A63V, S68T, M77T, | REYRSGDYWGQGTLVTVSS |
| | T79Y, R81Q, S82aN, | |
| | N82bS, K83R, G88A, V89L, | |
| | N99S) | |
| 153 | >F010302368 | EVQLVESGGGVVQPGGSLRLSCAASSRAFIRDVFT |
| | F0103464B09(L11V, T24A, | GWYRQAPGKQRELVARIYNGGNTNYADSVKGRFT |
| | T25S, R39Q, V40A, | ISRDNAKKTVYLQMNSLRPEDTALYYCLFSGTISTG |
| | E44Q, F62S, A63V, S68T, | REYRSGDYWGQGTLVTVSS |
| | M77T, T79Y, R81Q, | |
| | S82aN, N82bS, K83R, G88A, | |
| | V89L,N99S) | |
| 154 | >F010301656 | EVQLVESGGGLAQPGGSLRLSCAASGPVFNINRMA |
| | F0103387G04(K33R, S50Y, | WYRRAPGKQRELVAYVTPTGDISYTDSVKGRFTIS |
| | S56D, N93R) | RDGSKRWSLQMNSLTPEDTAVYYCRALLQPDSYS |
| | | NTRTYWGQGTLVTVSS |
| 155 | >F010301840 | EVQLVESGGGVVQPGGSLRLSCAASGPVFNINRMA |
| | F0103387G04(R76_V7 | WYRQAPGKQRELVAYVTPTGDISYTDSVKGRFTIS |
| | 8insT)(L11V, A12V, K33R, | RDGSKRTWSLQMNSLRPEDTALYYCRALLQPDSYS |
| | R39Q, S50Y, S56D, | NTRTYWGQGTLVTVSS |
| | T83R, V89L, N93R) | |
| 156 | >F010301841 | EVQLVESGGGVVQPGGSLRLSCAASGPVFNINRMA |
| | F0103387G04(L11V, A12V, | WYRQAPGKQRELVAYVTPTGDISYTDSVKGRFTIS |
| | K33R, R39Q, S50Y, | RDGSKRWSLQMNSLRPEDTALYYCRALLQPDSYS |
| | S56D, T83R, V89L, N93R) | NTRTYWGQGTLVTVSS |
| 157 | >F010301842 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRWSLQMNSLRPEDTALYYCRALLQPDSYS |
| | S56D, T60A, T83R, V89L, | NTRTYWGQGTLVTVSS |
| | N93R) | |
| 158 | >F010301843 | EVQLVESGGGVVQPGGSLRLSCAASGPVFNINRMA |
| | F0103387G04(L11V, A12V, | WYRQAPGKQRELVAYVTPTGDISYTDSVKGRFTIS |
| | K33R, R39Q, S50Y, | RDNSKRWSLQMNSLRPEDTALYYCRALLQPDSYS |
| | S56D, G73N, T83R, V89L, | NTRTYWGQGTLVTVSS |
| | N93R) | |
| 159 | >F010301844 | EVQLVESGGGVVQPGGSLRLSCAASGPVFNINRMA |
| | F0103387G04(L11V, A12V, | WYRQAPGKQRELVAYVTPTGDISYTDSVKGRFTIS |
| | K33R, R39Q, S50Y, | RDGSKNWSLQMNSLRPEDTALYYCRALLQPDSYS |
| | S56D, R76N, T83R, V89L, | NTRTYWGQGTLVTVSS |
| | N93R) | |
| 160 | >F010301845 | EVQLVESGGGVVQPGGSLRLSCAASGPVFNINRMA |
| | F0103387G04(L11V, A12V, | WYRQAPGKQRELVAYVTPTGDISYTDSVKGRFTIS |
| | K33R, R39Q, S50Y, | RDGSKRVSLQMNSLRPEDTALYYCRALLQPDSYSN |
| | S56D, W78V, T83R, V89L, | TRTYWGQGTLVTVSS |
| | N93R) | |
| 161 | >F010301846 | EVQLVESGGGVVQPGGSLRLSCAASGPVFNINRMA |
| | F0103387G04(L11V, A12V, | WYRQAPGKQRELVAYVTPTGDISYTDSVKGRFTIS |
| | K33R, R39Q, S50Y, | RDGSKRWYLQMNSLRPEDTALYYCRALLQPDSYS |
| | S56D, S79Y, T83R, V89L, | NTRTYWGQGTLVTVSS |
| | N93R) | |
| 162 | >F010301847 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDNSKNVYLQMNSLRPEDTALYYCRALLQPDSYS |
| | S56D, T60A, G73N, R76N, | NTRTYWGQGTLVTVSS |
| | W78V, S79Y, T83R, V89L, | |
| | N93R) | |
| 163 | >F010301848 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDNSKRVYLQMNSLRPEDTALYYCRALLQPDSYS |
| | S56D, T60A, G73N, W78V, | NTRTYWGQGTLVTVSS |
| | S79Y, T83R, V89L, N93R) | |
| 164 | >F010301865 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRVYLQMNSLRPEDTALYYCRALLQPDSYS |
| | S56D, T60A, W78V, S79Y, | NTRTYWGQGTLVTVS |
| | T83R, V89L, N93R) | |
| 165 | >F010301866 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKNVYLQMNSLRPEDTALYYCRALLQPDSYS |
| | S56D, T60A, R76N, W78V, | NTRTYWGQGTLVTVSS |
| | S79Y, T83R, V89L, N93R) | |
| 166 | >F010302310 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPRRYS |
| | S56D, T60A, G73A, W78V, | NTRTYWGQGTLVTVSS |
| | S79Y, T83R, V89L, N93R, | |
| | D99R, S100R)- | |
| | FLAG3-HIS6 | |
| 167 | >F010302311 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPRRYS |
| | S56D, T60A, G73R, W78V, | NTRTYWGQGTLVTVSS |
| | S79Y, T83R, V89L, N93R, | |
| | D99R, S100R)- | |
| | FLAG3-HIS6 | |
| 168 | >F010302312 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRVYLQMNSLRPEDTALYYCRALLQPDSYSI |
| | S56D, T60A, W78V, S79Y, | TRTYWGQGTLVTVSS |
| | T83R, V89L, N93R, N100cI) | |
| 169 | >F010302313 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPDSYSI |
| | S56D, T60A, G73A, W78V, | TRTYWGQGTLVTVSS |
| | S79Y, T83R, V89L, N93R, | |
| | N100cI) | |
| 170 | >F010302314 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPDSYSI |
| | S56D, T60A, G73R, W78V, | TRTYWGQGTLVTVSS |
| | S79Y, T83R, V89L, N93R, | |
| | N100cI) | |
| 171 | >F010302315 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRVYLQMNSLRPEDTALYYCRALLQPRSYSI |
| | S56D, T60A, W78V, S79Y, | TRTYWGQGTLVTVSS |
| | T83R, V89L, N93R, D99R, | |
| | N100cI) | |
| 172 | >F010302316 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPRSYSI |
| | S56D, T60A, G73A, W78V, | TRTYWGQGTLVTVSS |
| | S79Y, T83R, V89L, N93R, | |
| | D99R, N100cI)- | |
| | FLAG3-HIS6 | |
| 173 | >F010302317 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPRSYSI |
| | S56D, T60A, G73R, W78V, | TRTYWGQGTLVTVSS |
| | S79Y, T83R, V89L, N93R, | |
| | D99R, N100cI)- | |
| | FLAG3-HIS6 | |
| 174 | >F010302318 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRVYLQMNSLRPEDTALYYCRALLQPDRYSI |
| | S56D, T60A, W78V, S79Y, | TRTYWGQGTLVTVSS |
| | T83R, V89L, N93R, S100R, | |
| | N100cI) | |
| 175 | >F010302319 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPDRYSI |
| | S56D, T60A, G73A, W78V, | TRTYWGQGTLVTVSS |
| | S79Y, T83R, V89L, N93R, | |
| | S100R, N100cI)- | |
| | FLAG3-HIS6 | |
| 176 | >F010302320 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPDSYS |
| | S56D, T60A, G73A, W78V, | NTRTYWGQGTLVTVSS |
| | S79Y, T83R, V89L, N93R) | |
| 177 | >F010302321 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPDRYSI |
| | S56D, T60A, G73R, W78V, | TRTYWGQGTLVTVSS |
| | S79Y, T83R, V89L, N93R, | |
| | S100R,N100cI)- | |
| | FLAG3-HIS6 | |
| 178 | >F010302322 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, 50Y, | RDGSKRVYLQMNSLRPEDTALYYCRALLQPRRYSI |
| | S56D, T60A, W78V, S79Y, | TRTYWGQGTLVTVSS |
| | T83R, V89L, N93R, D99R, | |
| | S100R, N100cI)- | |
| | FLAG3-HIS6 | |
| 179 | >F010302323 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPRRYSI |
| | S56D, T60A, G73A, W78V, | TRTYWGQGTLVTVSS |
| | S79Y, T83R, V89L, N93R, | |
| | D99R, S100R, N100cI) | |
| 180 | >F010302324 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPRRYSI |
| | S56D, T60A, G73R, W78V, | TRTYWGQGTLVTVSS |
| | S79Y, T83R, V89L, N93R, | |
| | D99R, S100R, N100cI) | |
| 181 | >F010302325 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPDSYSN |
| | S56D, T60A, G73R, W78V, | TRTYWGQGTLVTVSS |
| | S79Y, T83R, V89L, N93R) | |
| 182 | >F010302326 | EVQLVESGGGVVQPGGSLRLSCAASGPVFNINRMA |
| | F0103387G04(L11V,A1 | WYRQAPGKQRELVAYVTPTGDISYADSVKGRFTIS |
| | 2V,K33R,R39Q,S50Y,S | RDGSKRVYLQMNSLRPEDTALYYCRALLQPRSYSN |
| | 56D,T60A, W78V,S79Y | TRTYWGQGTLVTVSS |
| | T83R, V89L,N93R,D99 | |
| | R) | |
| 183 | >F010302327 | EVQLVESGGGVVQPGGSLRLSCAASGPVFNINRMA |
| | F0103387G04(L11V,A1 | WYRQAPGKQRELVAYVTPTGDISYADSVKGRFTIS |
| | 2V,K33R,R39Q,S50Y,S | RDASKRVYLQMNSLRPEDTALYYCRALLQPRSYSN |
| | 56D,T60A, G73A, W78V | TRTYWGQGTLVTVSS |
| | ,S79Y,T83R,V89L,N93 | |
| | R,D99R) | |
| 184 | >F010302328 | EVQLVESGGGVVQPGGSLRLSCAASGPVFNINRMA |
| | F0103387G04(L11V,A1 | WYRQAPGKQRELVAYVTPTGDISYADSVKGRFTIS |
| | 2V,K33R,R39Q,S50Y,S | RDRSKRVYLQMNSLRPEDTALYYCRALLQPRSYSN |
| | 56D,T60A,G73R, W78V | TRTYWGQGTLVTVSS |
| | ,S79Y,T83R,V89L,N93 | |
| | R,D99R) | |
| 185 | >F010302329 | EVQLVESGGGVVQPGGSLRLSCAASGPVFNINRMA |
| | F0103387G04(L11V,A1 | WYRQAPGKQRELVAYVTPTGDISYADSVKGRFTIS |
| | 2V,K33R,R39Q,S50Y,S | RDGSKRVYLQMNSLRPEDTALYYCRALLQPDRYS |
| | 56D,T60A,W78V,S79Y | NTRTYWGQGTLVTVSS |
| | T83R,V89L,N93R,S10 | |
| | 0R) | |
| 186 | >F010302330 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDASKRVYLQMNSLRPEDTALYYCRALLQPDRYS |
| | 5S6D, T60A, G73A, W78V, | NTRTYWGQGTLVTVSS |
| | S79Y, T83R, V89L, N93R, | |
| | S100R) | |
| 187 | >F010302331 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPDRYS |
| | S56D, T60A, G73R, W78V, | NTRTYWGQGTLVTVSS |
| | S79Y, T83R, V89L, N93R, | |
| | S100R) | |
| 188 | >F010302332 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDGSKRVYLQMNSLRPEDTALYYCRALLQPRRYS |
| | S56D, T60A, W78V, S79Y, | NTRTYWGQGTLVTVSS |
| | T83R, V89L, N93R, D99R, | |
| | S100R) | |
| 189 | >F010302370 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPSSYSI |
| | S56D, T60A, G73R, W78V, | TRTYWGQGTLVTVSS |
| | S79Y, T83R, V89L, N93R, | |
| | D99S, N100cI) | |
| 190 | >F010302371 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPNVYSI |
| | S56D, T60A, G73R, W78V, | TRTYWGQGTLVTVSS |
| | S79Y, T83R, V89L, N93R, | |
| | D99N, S100V, N100cI) | |
| 191 | >F010302372 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RDRSKRVYLQMNSLRPEDTALYYCRALLQPDVYSI |
| | S56D, T60A, G73R, W78V, | TRTYWGQGTLVTVSS |
| | S79Y, T83R, V89L, N93R, | |
| | S100V, N100cI) | |
| 192 | >F010302383 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RGGSKRVYLQMNSLRPEDTALYYCRALLQPSSYSG |
| | S56D, T60A, D72G, W78V, | TRTYWGQGTLVTVSS |
| | S79Y, T83R, V89L, N93R, | |
| | D99S, N100cG) | |
| 193 | >F010302384 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RGGSKRVYLQMNSLRPEDTALYYCRALLQPSSYSI |
| | S56D, T60A, D72G, W78V, | TRTYWGQGTLVTVSS |
| | S79Y, T83R, V89L, N93R, | |
| | D99S, N100cI) | |
| 194 | >F010302385 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RQGSKRVYLQMNSLRPEDTALYYCRALLQPSSYSG |
| | S56D, T60A, D72Q, W78V, | TRTYWGQGTLVTVSS |
| | S79Y, T83R, V89L, N93R, | |
| | D99S, N100cG) | |
| 195 | >F010302386 | EVQLVESGGGVVQPGGSLRLSCAASGPVFNINRMA |
| | F0103387G04(L11V, A12V, | WYRQAPGKQRELVAYVTPTGDISYADSVKGRFTIS |
| | K33R, R39Q, S50Y, | RQGSKRVYLQMNSLRPEDTALYYCRALLQPSSYSI |
| | S56D, T60A, D72Q, W78V, | TRTYWGQGTLVTVSS |
| | S79Y, T83R, V89L, N93R, | |
| | D99S, N100cI) | |
| 196 | F0103275B05-CDR1 | GSIFNINSMA |
| 197 | F0103275B05-CDR1-A | GSIFNINRMA |
| 198 | F0103275B05-CDR2 | SSTNGGSTN |
| 199 | F0103275B05-CDR2-A | YSTNGGDTN |
| 200 | F0103275B05-CDR3 | LLQPSIYDISRTY |
| 201 | F0103387G05-CDR1 | GRILRIGYMR |
| 202 | F0103387G05-CDR2 | RITDDSATD |
| 203 | F0103387G05-CDR2-A | RITGGSATG |
| 204 | F0103387G05-CDR2-B | RITDDSATG |
| 205 | F0103387G05-CDR2-C | RITGGSATG |
| 206 | F0103387G05-CDR3 | LVTASVRGGSIHSGTY |
| 207 | F0103464B09-CDR1 | SRAFIRDVFTG |
| 208 | F0103464B09-CDR1-A | SRAFIRDLFTG |
| 209 | F0103464B09-CDR1-B | SRQFIRDVFTG |
| 210 | F0103464B09-CDR1-C | HRQFIRDVFTG |
| 211 | F0103464B09-CDR1-D | HRAFIRDVFTG |
| 212 | F0103464B09-CDR1-E | HRAFIRDLFTG |
| 213 | F0103464B09-CDR2 | RIYNGGNTN |
| 214 | F0103464B09-CDR2-A | RIYNEGNTN |
| 215 | F0103464B09-CDR2-B | RIYNEGNTQ |
| 216 | F0103464B09-CDR2-C | RIYESGNTQ |
| 217 | F0103464B09-CDR2-D | RIYESGNTN |
| 218 | F0103464B09-CDR2-E | RIYNEGNTN |
| 219 | F0103464B09-CDR3 | SGTINTGREYRSGDY |
| 220 | F0103464B09-CDR3-A | SGTISTGREYRSGDY |
| 221 | F0103387G04-CDR1 | GPVFNINKMA |
| 222 | F0103387G04-CDR1-A | GPVFNINRMA |
| 223 | F0103387G04-CDR2 | SVTPTGSIS |
| 224 | F0103387G04-CDR2-A | YVTPTGDIS |
| 225 | F0103387G04-CDR3 | LLQPDSYSNTRTY |
| 226 | F0103387G04-CDR3-A | LLQPRRYSNTRTY |
| 227 | F0103387G04-CDR3-B | LLQPDSYSITRTY |
| 228 | F0103387G04-CDR3-C | LLQPRSYSITRTY |
| 229 | F0103387G04-CDR3-B | LLQPRSYSNTRTY |
| 230 | F0103387G04-CDR3-E | LLQPSSYSITRTY |
| 231 | F0103387G04-CDR3-F | LLQPNVYSITRTY |
| 232 | F0103387G04-CDR3-G | LLQPDVYSITRTY |
| 233 | F0103387G04-CDR3-H | LLQPSSYSGTRTY |
| 234 | ALB11002 (“ALB201”) | EVQLVESGGGXVQPGNSLRLSCAASGFTFSSFGMS |
| | without C- | WVRQAPGKGLEWVSSISGSGSDTLYADSVKGRFTI |
| | terminal A, X11 is L or | SRDNAKTTLYLQMNSLRPEDTAXYYCTIGGSLSRS |
| | V, X93 is V or L | SQGTLVTVSS |
| 235 | HSA-CDR1 | GFTFSSFGMS |
| 236 | HSA-CDR2 | SISGSGSDTL |
| 237 | HSA-CDR3 | GGSLSR |
| 238 | ALB11002 (“ALB201”), | EVQLVESGGGXVQPGNSLRLSCAASGFTFSSFGMS |
| | X11 is L or | WVRQAPGKGLEWVSSISGSGSDTLYADSVKGRFTI |
| | V, X93 is V or L | SRDNAKTTLYLQMNSLRPEDTAXYYCTIGGSLSRS |
| | | SQGTLVTVSSA |
| 239 | ALB11002 (“ALB201”) | DVQLVESGGGVVQPGNSLRLSCAASGFTFSSFGMS |
| | E1D L11V | WVRQAPGKGLEWVSSISGSGSDTLYADSVKGRFTI |
| | L93V without C- | SRDNAKTTLYLQMNSLRPEDTALYYCTIGGSLSRSS |
| | terminal A | QGTLVTVSS |
| 240 | ALB11002 (“ALB201”) | DVQLVESGGGVVQPGNSLRLSCAASGFTFSSFGMS |
| | E1D L11V | WVRQAPGKGLEWVSSISGSGSDTLYADSVKGRFTI |
| | L93V | SRDNAKTTLYLQMNSLRPEDTALYYCTIGGSLSRSS |
| | | QGTLVTVSSA |
| 241 | AB11 without C- | EVQLVESGGGLVQPGNSLRLSCAASGFTFSSFGMS |
| | terminal A | WVRQAPGKGLEWVSSISGSGSDTLYADSVKGRFTI |
| | | SRDNAKTTLYLQMNSLRPEDTAVYYCTIGGSLSRS |
| | | SQGTLVTVSS |
| 242 | AB11 | EVQLVESGGGLVQPGNSLRLSCAASGFTFSSFGMS |
| | | WVRQAPGKGLEWVSSISGSGSDTLYADSVKGRFTI |
| | | SRDNAKTTLYLQMNSLRPEDTAVYYCTIGGSLSRS |
| | | SQGTLVTVSS |
| 243 | 9GS-linker | GGGGSGGGS |
| 244 | 20GS linker | GGGGSGGGGSGGGGSGGGGS |
| 245 | 35GS linker | GGGGSGGGGSGGGGSGGGGSGGGGSGGGGSGGG |
| | | GS |
| 246 | Linker subunit | GGGS |
| 247 | F0103262B06-CDR1 | TRTFSTYAMG |
| 248 | F0103262B06-CDR2 | HINFSGSSTRY |
| 249 | F0103262B06-CDR3 | ARWVAGPPRYDYEY |
| 250 | F0103262C02-CDR1 | GLPFGLYILG |
| 251 | F0103262C02-CDR2 | AISRSGRDTV |
| 252 | F0103262C02-CDR3 | DSVPRGTPTITESEYAI |
| 253 | F0103265A11-CDR1 | GMLFNANTQG |
| 254 | F0103265A11-CDR2 | FIFSGGYTN |
| 255 | F0103265A11-CDR3 | SRY |
| 256 | F0103265B04-CDR1 | SFIFSNNYME |
| 257 | F0103265B04-CDR2 | RITGRGNTN |
| 258 | F0103265B04-CDR3 | LWYGGRA |
| 259 | F0103362B08-CDR1 | VRPFSTSAMG |
| 260 | F0103362B08-CDR2 | GILWNGIVTY |
| 261 | F0103362B08-CDR3 | DRDYGGRSFSAYEYEY |
| 262 | F0103454D07-CDR1 | GGIININYIA |
| 263 | F0103454D07-CDR2 | RISSDDTIK |
| 264 | F0103454D07-CDR3 | LITPWTGDTRTY |
| 265 | ALB00223 | EVQLVESGGGVVQPGGSLRLSCAASGFTFRSFGMS |
| | | WVRQAPGKGPEWVSSISGSGSDTLYADSVKGRFTI |
| | | SRDNSKNTLYLQMNSLRPEDTALYYCTIGGSLSRSS |
| | | QGTLVTVSSA |
| 266 | ALB00223 without C- | EVQLVESGGGVVQPGGSLRLSCAASGFTFRSFGMS |
| | terminal A | WVRQAPGKGPEWVSSISGSGSDTLYADSVKGRFTI |
| | | SRDNSKNTLYLQMNSLRPEDTALYYCTIGGSLSRSS |
| | | QGTLVTVSS |
| 267 | ALB00223-HSA-CDR1 | GFTFRSFGMS |
| 268 | VHH2-consensus | QVQLVESGGGLVQAGGSLRLSCAAS |
| | Framework 1 | |
| 269 | VHH2-consensus | WYRQAPGKQRELVA |
| | Framework 2 | |
| 270 | VHH2-consensus | YADSVKGRFTISRDNAKNTLYLQMNSLKPEDTAVY |
| | Framework 3 | YCNA |
| 271 | VHH2-consensus | WGQGTLVTVSS |
| | Framework 4 | |
| 272 | Nucleotide sequence | GCGGCCGCAGATTATAAAGATCATGATGGCGATT |
| | encoding FLAG-HIS6 | ATAAAGATCATGATATTGATTATAAAGATGATGA |
| | peptide | TGATAAAGGGGCCGCACATCATCATCATCATCAT |
| 273 | Nucleotide sequence | GAGGTGCAATTGGTGGAGTCTGGGGGAGGTTTG |
| | encoding F0103262B06 | GTGCAGCCTGGGGGCTCTCTGAGACTCTCGTGTG |
| | | CTGGGTCTACACGCACGTTTAGCACCTATGCCAT |
| | | GGGCTGGTTCCGCCAGGCTCCAGGGAGGGAGCG |
| | | TGAGTTTGTAGCACATATTAATTTTAGCGGTAGT |
| | | AGCACAAGGTATGCAGACTCCGTGAAGGGCCGA |
| | | TTCACCATCTCCAGAGACAACGCCAAGAATATGG |
| | | GATATCTGCAGATGAATAGCCTGAAACCTGAGG |
| | | ACACGGCCGTTTATTATTGTGCAGCCCGGTGGGT |
| | | CGCTGGCCCTCCGAGGTATGACTATGAGTACTGG |
| | | GGCCAGGGGACCCTGGTCACCGTCTCCTCA |
| 274 | Nucleotide sequence | GAGGTGCAATTGGTGGAGTCTGGGGGAGGATTG |
| | encoding F0103262C02 | GTGCAGGCTGGGGGCTCTCTGACACTCTCCTGTG |
| | | CAGCCTCTGGTCTGCCCTTCGGATTGTATATTCTG |
| | | GGCTGGATCCGCCGGGCTCCAGGGAAGGAGCGT |
| | | GATTTTGTAGCAGCTATTAGCCGGAGTGGTAGGG |
| | | ACACGGTTTATGCAAACTCCGTGAAGGGCCGATT |
| | | CACCATCTCCAGAGACAACGCCAAGAACATGGT |
| | | GTACCTGCGAATGGACAATCTGAGACCGGAGGA |
| | | CACGGCCGCATATTACTGTGCAGTGGACTCAGTG |
| | | CCACGCGGAACTCCTACCATCACAGAGTCTGAGT |
| | | ACGCCATCTGGGGCCAGGGGACCCTGGTCACCGT |
| | | CTCCTCA |
| 275 | Nucleotide sequence | GAGGTGCAATTGGTGGAGTCTGGGGGAGGCTTG |
| | encoding F0103265A11 | GTGCAGCCTGGAGGGTCTCTGAGACTCTCCTGTG |
| | | CAGCCTCTGGAATGCTCTTCAACGCCAATACCCA |
| | | GGGCTGGTACCGCCAGGCTCCAGGGAAGCAGCG |
| | | CGAATTGGTCGCATTTATTTTTAGTGGTGGTTAC |
| | | ACAAACTATGTAGACTCCGTGAAGGGCCGTTTCA |
| | | CCATCTCCAGAGACAACGCCAAGCGCACAATGT |
| | | ATCTGCAGATGAACAGCCTGAAACCTGAGGACT |
| | | CGGCCATCTATTACTGCTCATTGAGTCGCTACTT |
| | | GGGCCAGGGGACCCTGGTCACCGTCTCCTCA |
| 276 | Nucleotide sequence | GAGGTGCAATTGGTGGAGTCTGGGGGAGGCTTG |
| | encoding F0103265B04 | GTGCAGCCTGGGGGGTCTCTGAGACTCTCCTGTG |
| | | CAGCCCCTAGTTTCATCTTCAGCAACAATTACAT |
| | | GGAGTGGTACCGGCAGGCTCCAGGGAAGCAGCG |
| | | CGACTGGGTCGCACGTATTACAGGTCGCGGTAAC |
| | | ACAAACTATCTGGACTCCGTGAAGGGCCGATTCA |
| | | CCATCTCCAGAGACGACGCCAAGAATACGGTGT |
| | | ATCTAGAAATCGACAGCCTGAAACCTGAGGACA |
| | | CGGCCGTCTATTACTGTAGTGCACTCTGGTACGG |
| | | CGGGCGCGCATGGGGCAAAGGGACCCTGGTCAC |
| | | CGTCTCCTCA |
| 277 | Nucleotide sequence | GAGGTGCAATTGGTGGAGTCTGGGGGAGGCTTG |
| | encoding F0103275B05 | GTGCAGCCTGGGGGGTCTCTGAGACTCTCCTGTG |
| | | CAGCCTCTGGAAGCATCTTCAATATCAACAGTAT |
| | | GGCCTGGTATCGCCGGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCTAGAGACAACGCCAAACGGGTGTATC |
| | | TGCAAATGAACAGCCTGACACCTGAGGACACGG |
| | | CCGTCTATTATTGTAATGCACTGCTACAACCGTC |
| | | GATTTATGACATTAGTCGCACATATTGGGGCCAG |
| | | GGGACCCTGGTCACCGTCTCCTCA |
| 278 | Nucleotide sequence | GAGGTGCAATTGGTGGAGTCTGGGGGAGGATTG |
| | encoding F0103362B08 | GTGCAGGCTGGGGGCTCTCTGAGACTCTCCTGTG |
| | | CAGCCTCTGTACGTCCCTTCAGTACCTCAGCCAT |
| | | GGGCTGGTTCCGCCAGGCTCCAGAGAAGGAGCG |
| | | TGAGGCTGTAGCAGGTATTCTGTGGAATGGTATT |
| | | GTCACATACTATGCAGACTCCGTGAAGGGCCGAT |
| | | TCACCATCTCCAGAGACAACGCCAAGAATGAAG |
| | | TATATCTGCAAATGAACAAACTGAAACCCGAGG |
| | | ACACGGCCGTTTATTATTGTGCATTAGATAGAGA |
| | | TTATGGTGGGCGATCTTTTTCGGCATATGAATAT |
| | | GAGTACTGGGGCCAGGGGACCCTGGTCACCGTCT |
| | | CCTCA |
| 279 | Nucleotide sequence | GAGGTGCAATTGGTGGAGTCTGGGGGAGGCTTG |
| | encoding F0103387G04 | GCGCAGCCTGGGGGGTCTCTGAGACTCTCCTGTG |
| | | CAGCCTCTGGACCCGTCTTCAATATCAACAAGAT |
| | | GGCCTGGTACCGCCGGGCTCCAGGGAAGCAGCG |
| | | CGAATTGGTCGCAAGTGTCACCCCTACTGGTAGT |
| | | ATAAGTTATACTGACTCCGTGAAGGGCCGATTCA |
| | | CCATTTCTAGAGACGGCTCCAAGCGGTGGTCTCT |
| | | ACAAATGAACAGCCTGACACCTGAGGACACGGC |
| | | CGTCTATTACTGTAACGCTTTACTACAACCGGAT |
| | | AGTTATTCTAATACGCGCACATATTGGGGCCAGG |
| | | GGACCCTGGTCACCGTCTCCTCA |
| 280 | Nucleotide sequence | GAGGTGCAATTGGTGGAGTCTGGGGGAGGCTTG |
| | encoding F0103387G05 | GTGCAGGCTGGGGGGTCACTGAGACTCTCCTGTG |
| | | ACGCCTCTGGAAGGATCCTCCGTATCGGCTACAT |
| | | GAGGTGGCACCGCCAGGGTGCAGGGAAGCAGCG |
| | | CGAGTTTGTCGCGCGTATTACTGATGATAGTGCT |
| | | ACAGACTATGCAGACTCCGTGAAGGGCCGATTC |
| | | ACCATCTCCAGAGACAACGCCAAGAACACGGTG |
| | | TATCTGCAAATGAACAACCTGAATCCTGAGGACA |
| | | CGGCCGTCTATTATTGTGAGGCGTTGGTGACTGC |
| | | GAGTGTACGTGGTGGGAGTATACATTCTGGTACC |
| | | TATTGGGGCCGGGGGACCCTGGTCACCGTCTCCT |
| | | CA |
| 281 | Nucleotide sequence | GAGGTGCAATTGGTGGAGTCTGGGGGAGGATTG |
| | encoding F0103454D07 | GTGCAGCCTGGAGGATCACTTAGACTGTCCTGTG |
| | | TAGCCTCCGGGGGCATCATCAATATCAATTACAT |
| | | TGCCTGGTACCGCCAGACTCCAGGGAAGCAGCG |
| | | CGACTTGGTCGCTCGTATTAGTAGTGATGATACA |
| | | ATAAAGTATGGCGACTCCGTGAAGGGCCGATTC |
| | | GCCATGTCCAGAGACAAGGTCAAGAATATGGTG |
| | | CATCTACAAATGAACAGCCTGACTACCGAGGAC |
| | | ACAGGTGTCTATGTCTGTTCAGCCCTCATCACGC |
| | | CTTGGACAGGAGACACCCGGACCTATTGGGGCC |
| | | GGGGGACCCTGGTCACCGTCTCCTCA |
| 282 | Nucleotide sequence | GAGGTGCAATTGGTGGAGTCTGGGGGAGGATTG |
| | encoding F0103464B09 | GTGCAGCCTGGAGGATCACTAAGACTGTCCTGTG |
| | | CAACAACCTCTAGAGCTTTCATCAGGGACGTTTT |
| | | CACGGGCTGGTATCGCCGGGTTCCCGGGAAGGA |
| | | GCGCGAATTGGTCGCTCGCATTTACAATGGCGGT |
| | | AACACAAATTATGCAGACTTCGCGAAGGGCCGA |
| | | TTCTCCATCTCCAGGGACAACGCCAAGAAGATGG |
| | | TGACTCTGAGAATGAGCAATCTGAAACCTGAGG |
| | | ACACAGGGGTCTATTACTGCCTTTTTTCGGGTAC |
| | | AATCAATACTGGCAGAGAGTATCGGTCTGGAGA |
| | | CTACTGGGGCCAGGGGACCCTGGTCACCGTCTCC |
| | | TCA |
| 283 | Nucleotide sequence | GAGGTGCAATTGGTGGAGTCTGGGGGAGGCTTG |
| | encoding F0103464B09 | GTGCAGCCTGGGGGGTCTCTGAGACTCTCCTGTG |
| | | CAGCCTCTGGAAGCATCTTCAATATCAACCGCAT |
| | | GGCCTGGTATCGCCGGGCTCCAGGGAAGCAGCG |
| | | CGAATTGGTCGCATATAGCACCAATGGTGGTGAT |
| | | ACAAACTATGCAGACTCCGTGAAGGGCCGATTC |
| | | ACCATCAGCAGAGACAACGCCAAACGGGTGTAT |
| | | CTGCAAATGAACAGCCTGACACCTGAGGACACG |
| | | GCCGTCTATTATTGTCGCGCACTGCTACAACCGT |
| | | CGATTTATGACATTAGTCGCACATATTGGGGCCA |
| | | GGGGACCCTGGTCACCGTCTCCTCA |
| 284 | Nucleotide sequence | GAGGTGCAATTGGTGGAGTCTGGGGGAGGCGTT |
| | encoding F010301635 | GTGCAGCCTGGGGGGTCTCTGAGACTCTCCTGTG |
| | | CAGCCTCTGGAAGCATCTTCAATATCAACAGTAT |
| | | GGCCTGGTATCGCCAGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAACGGGTGTAT |
| | | CTGCAAATGAACAGCCTGCGCCCTGAGGACACG |
| | | GCCCTGTATTATTGTAATGCACTGCTACAACCGT |
| | | CGATTTATGACATTAGTCGCACATATTGGGGCCA |
| | | GGGGACCCTGGTCACCGTCTCCTCA |
| 285 | Nucleotide sequence | GAGGTGCAATTGGTGGAGTCTGGGGGAGGCGTT |
| | encoding F010301636 | GTGCAGCCTGGGGGGTCTCTGAGACTCTCCTGTG |
| | | CAGCCTCTGGAAGCATCTTCAATATCAACAGTAT |
| | | GGCCTGGTATCGCCGGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAAAACGTGTAT |
| | | CTGCAAATGAACAGCCTGCGCCCTGAGGACACG |
| | | GCCCTGTATTATTGTAATGCACTGCTACAACCGT |
| | | CGATTTATGACATTAGTCGCACATATTGGGGCCA |
| | | GGGGACCCTGGTCACCGTCTCCTCA |
| 286 | Nucleotide sequence | GAGGTGCAATTGGTGGAGTCTGGGGGAGGCGTT |
| | encoding F010301637 | GTGCAGCCTGGGGGGTCTCTGAGACTCTCCTGTG |
| | | CAGCCTCTGGAAGCATCTTCAATATCAACAGTAT |
| | | GGCCTGGTATCGCCGGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAACGGGTGTAT |
| | | CTGCAAATGAACAGCCTGCGCCCTGAGGACACG |
| | | GCCCTGTATTATTGTAATGCACTGCTACAACCGT |
| | | CGATTTATGACATTAGTCGCACATATTGGGGCCA |
| | | GGGGACCCTGGTCACCGTCTCCTCA |
| 287 | Nucleotide sequence | GAGGTGCAATTGGTGGAGTCTGGGGGAGGCGTT |
| | encoding F010301638 | GTGCAGCCTGGGGGGTCTCTGAGACTCTCCTGTG |
| | | CAGCCTCTGGAAGCATCTTCAATATCAACAGTAT |
| | | GGCCTGGTATCGCCAGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAAAACGTGTAT |
| | | CTGCAAATGAACAGCCTGCGCCCTGAGGACACG |
| | | GCCCTGTATTATTGTAATGCACTGCTACAACCGT |
| | | CGATTTATGACATTAGTCGCACATATTGGGGCCA |
| | | GGGGACCCTGGTCACCGTCTCCTCA |
| 288 | Nucleotide sequence | GAGGTGCAATTGGTGGAGTCTGGGGGAGGCGTT |
| | encoding F010301639 | GTGCAGCCTGGGGGGTCTCTGAGACTCTCCTGTG |
| | | CAGCCTCTGGAAGCATCTTCAATATCAACAGTAT |
| | | GGCCTGGTATCGCCAGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAACGGACCGTG |
| | | TATCTGCAAATGAACAGCCTGCGCCCTGAGGACA |
| | | CGGCCCTGTATTATTGTAATGCACTGCTACAACC |
| | | GTCGATTTATGACATTAGTCGCACATATTGGGGC |
| | | CAGGGGACCCTGGTCACCGTCTCCTCA |
| 289 | Nucleotide sequence | GAGGTGCAATTGGTGGAGTCTGGGGGAGGCGTT |
| | encoding F010301640 | GTGCAGCCTGGGGGGTCTCTGAGACTCTCCTGTG |
| | | CAGCCTCTGGAAGCATCTTCAATATCAACAGTAT |
| | | GGCCTGGTATCGCCGGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAACGGACCGTG |
| | | TATCTGCAAATGAACAGCCTGCGCCCTGAGGACA |
| | | CGGCCCTGTATTATTGTAATGCACTGCTACAACC |
| | | GTCGATTTATGACATTAGTCGCACATATTGGGGC |
| | | CAGGGGACCCTGGTCACCGTCTCCTCA |
| 290 | Nucleotide sequence | GAGGTGCAATTGGTGGAGTCTGGGGGAGGCGTT |
| | encoding F010301641 | GTGCAGCCTGGGGGGTCTCTGAGACTCTCCTGTG |
| | | CAGCCTCTGGAAGCATCTTCAATATCAACAGTAT |
| | | GGCCTGGTATCGCCGGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAAAACACCGTG |
| | | TATCTGCAAATGAACAGCCTGCGCCCTGAGGACA |
| | | CGGCCCTGTATTATTGTAATGCACTGCTACAACC |
| | | GTCGATTTATGACATTAGTCGCACATATTGGGGC |
| | | CAGGGGACCCTGGTCACCGTCTCCTCA |
| 291 | Nucleotide sequence | GAGGTGCAATTGGTGGAGTCTGGGGGAGGCGTT |
| | encoding F010301642 | GTGCAGCCTGGGGGGTCTCTGAGACTCTCCTGTG |
| | | CAGCCTCTGGAAGCATCTTCAATATCAACAGTAT |
| | | GGCCTGGTATCGCCAGGCTCCAGGGAAGCAGCG |
| | | CGAATTGGTCGCAAGTAGCACCAATGGTGGTAGT |
| | | ACAAACTATGCAGACTCCGTGAAGGGCCGATTC |
| | | ACCATCTCCAGAGACAACGCCAAAAACACCGTG |
| | | TATCTGCAAATGAACAGCCTGCGCCCTGAGGACA |
| | | CGGCCCTGTATTATTGTAATGCACTGCTACAACC |
| | | GTCGATTTATGACATTAGTCGCACATATTGGGGC |
| | | CAGGGGACCCTGGTCACCGTCTCCTCA |
| 292 | Nucleotide sequence | GAGGTGCAATTGGTGGAGTCTGGGGGAGGCGTT |
| | encoding F010301652 | GTGCAGCCTGGGGGGTCTCTGAGACTCTCCTGTG |
| | | CAGCCTCTGGAAGCATCTTCAATATCAACCGCAT |
| | | GGCCTGGTATCGCCGGGCTCCAGGGAAGCAGCG |
| | | CGAATTGGTCGCATATAGCACCAATGGTGGTGAT |
| | | ACAAACTATGCAGACTCCGTGAAGGGCCGATTC |
| | | ACCATCTCCAGAGACAACGCCAAAAACGTGTAT |
| | | CTGCAAATGAACAGCCTGCGCCCTGAGGACACG |
| | | GCCCTGTATTATTGTCGCGCACTGCTACAACCGT |
| | | CGATTTATGACATTAGTCGCACATATTGGGGCCA |
| | | GGGGACCCTGGTCACCGTCTCCTCA |
| 293 | Nucleotide sequence | GAGGTGCAATTGGTGGAGTCTGGGGGAGGCGTT |
| | encoding F010301653 | GTGCAGCCTGGGGGGTCTCTGAGACTCTCCTGTG |
| | | CAGCCTCTGGAAGCATCTTCAATATCAACCGCAT |
| | | GGCCTGGTATCGCCGGGCTCCAGGGAAGCAGCG |
| | | CGAATTGGTCGCATATAGCACCAATGGTGGTGAT |
| | | ACAAACTATGCAGACTCCGTGAAGGGCCGATTC |
| | | ACCATCTCCAGAGACAACGCCAAACGGGTGTAT |
| | | CTGCAAATGAACAGCCTGCGCCCTGAGGACACG |
| | | GCCCTGTATTATTGTCGCGCACTGCTACAACCGT |
| | | CGATTTATGACATTAGTCGCACATATTGGGGCCA |
| | | GGGGACCCTGGTCACCGTCTCCTCA |
| 294 | Nucleotide sequence | GAGGTGCAATTGGTGGAGTCTGGGGGAGGCGTT |
| | encoding F010301654 | GTGCAGCCTGGGGGGTCTCTGAGACTCTCCTGTG |
| | | CAGCCTCTGGAAGCATCTTCAATATCAACCGCAT |
| | | GGCCTGGTATCGCCAGGCTCCAGGGAAGCAGCG |
| | | CGAATTGGTCGCATATAGCACCAATGGTGGTGAT |
| | | ACAAACTATGCAGACTCCGTGAAGGGCCGATTC |
| | | ACCATCTCCAGAGACAACGCCAAACGGGTGTAT |
| | | CTGCAAATGAACAGCCTGCGCCCTGAGGACACG |
| | | GCCCTGTATTATTGTCGCGCACTGCTACAACCGT |
| | | CGATTTATGACATTAGTCGCACATATTGGGGCCA |
| | | GGGGACCCTGGTCACCGTCTCCTCA |
| 295 | Nucleotide sequence | GAGGTGCAATTGGTGGAGTCTGGGGGAGGCGTT |
| | encoding F010301655 | GTGCAGCCTGGGGGGTCTCTGAGACTCTCCTGTG |
| | | CAGCCTCTGGAAGCATCTTCAATATCAACCGCAT |
| | | GGCCTGGTATCGCCAGGCTCCAGGGAAGCAGCG |
| | | CGAATTGGTCGCATATAGCACCAATGGTGGTGAT |
| | | ACAAACTATGCAGACTCCGTGAAGGGCCGATTC |
| | | ACCATCTCCAGAGACAACGCCAAAAACGTGTAT |
| | | CTGCAAATGAACAGCCTGCGCCCTGAGGACACG |
| | | GCCCTGTATTATTGTCGCGCACTGCTACAACCGT |
| | | CGATTTATGACATTAGTCGCACATATTGGGGCCA |
| | | GGGGACCCTGGTCACCGTCTCCTCA |
| 296 | Nucleotide sequence | GAGGTGCAATTGGTGGAGTCTGGGGGAGGCTTGGTGC |
| | encoding F010301556 | AGGCTGGGGGGTCACTGAGACTCTCCTGTGCTGCCTC |
| | | TGGAAGAATCCTCCGTATCGGCTACATGAGGTGGCAC |
| | | CGCCAGGGTGCAGGGAAGCAGCGCGAGTTTGTCGCG |
| | | CGTATTACTGGTGGTAGTGCTACAGGCTATGCAGACT |
| | | CCGTGAAGGGCCGATTCACCATCTCCAGAGACAACGC |
| | | CAAGAACACGGTGTATCTGCAAATGAACAACCTGAAT |
| | | CCTGAGGACACGGCCGTCTATTATTGTGAGGCGTTGG |
| | | TGACTGCGAGTGTACGTGGTGGGAGTATACATTCTGG |
| | | AACCTATTGGGGCCGGGGGACCCTGGTCACCGTCTCC |
| | | TCA |
| 297 | Nucleotide sequence | GAGGTGCAATTGGTGGAGTCTGGGGGAGGCTTGGTGC |
| | encoding F010301563 | AGGCTGGGGGGTCACTGAGACTCTCCTGTGCTGCCTC |
| | | TGGAAGAATCCTCCGTATCGGCTACATGAGGTGGCAC |
| | | CGCCAGGGTGCAGGGAAGCAGCGCGAGTTTGTCGCG |
| | | CGTATTACTGATGATAGTGCTACAGGCTATGCAGACT |
| | | CCGTGAAGGGCCGATTCACCATCTCCAGAGACAACGC |
| | | CAAGAACACGGTGTATCTGCAAATGAACAACCTGAAT |
| | | CCTGAGGACACGGCCGTCTATTATTGTGAGGCGTTGG |
| | | TGACTGCGAGTGTACGTGGTGGGAGTATACATTCTGG |
| | | AACCTATTGGGGCCGGGGGACCCTGGTCACCGTCTCC |
| | | TCA |
| 298 | Nucleotide sequence | GAGGTGCAATTGGTGGAGTCTGGGGGAGGCGTGGTG |
| | encoding F010301849 | CAGCCTGGGGGGTCACTGAGACTCTCCTGTGATGCCT |
| | | CTGGAAGGATACTCCGTATCGGCTACATGAGGTGGCA |
| | | CCGCCAGGGTGCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 299 | Nucleotide sequence | GAGGTGCAATTGGTGGAGTCTGGGGGAGGCGTCGTG |
| | encoding F010301850 | CAGCCGGGGGGGTCACTGAGACTCTCCTGTGCGGCCA |
| | | GCGGAAGGATTCTCCGTATCGGCTACATGAGGTGGTA |
| | | TCGCCAGGCGCCGGGGAAGCAGCGCGAGTTTGTCGC |
| | | GCGTATTACTGATGATAGTGCTACAGGTTATGCAGAC |
| | | TCCGTGAAGGGCCGATTCACCATCTCCAGAGACAACG |
| | | CCAAGAACACGGTGTATCTGCAAATGAACAGCCTGCG |
| | | CCCTGAGGACACGGCCCTGTATTATTGTGAGGCGTTG |
| | | GTGACTGCGAGTGTACGTGGTGGGAGTATACATTCTG |
| | | GCACCTATTGGGGCCAGGGGACCCTGGTCACCGTCTC |
| | | CTCA |
| 300 | Nucleotide sequence | GAGGTGCAATTGGTGGAGTCTGGGGGAGGCGTGGTG |
| | encoding F010301643 | CAGCCTGGGGGGTCACTGAGACTCTCCTGTGCCGCCT |
| | | CTGGAAGGATACTCCGTATCGGCTACATGAGGTGGCA |
| | | CCGCCAGGGTGCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 301 | Nucleotide sequence | GAGGTGCAATTGGTGGAGTCTGGGGGAGGCGTGGTG |
| | encoding F010301644 | CAGCCTGGGGGGTCACTGAGACTCTCCTGTGATGCCT |
| | | CTGGAAGGATACTCCGTATCGGCTACATGAGGTGGTA |
| | | TCGCCAGGGTGCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 302 | Nucleotide sequence | GAGGTGCAATTGGTGGAGTCTGGGGGAGGCGTGGTG |
| | encoding F010301645 | CAGCCTGGGGGGTCACTGAGACTCTCCTGTGATGCCT |
| | | CTGGAAGGATACTCCGTATCGGCTACATGAGGTGGCA |
| | | CCGCCAGGCTGCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 303 | Nucleotide sequence | GAGGTGCAATTGGTGGAGTCTGGGGGAGGCGTGGTG |
| | encoding F010301646 | CAGCCTGGGGGGTCACTGAGACTCTCCTGTGATGCCT |
| | | CTGGAAGGATACTCCGTATCGGCTACATGAGGTGGCA |
| | | CCGCCAGGGTCCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 304 | Nucleotide sequence | GAGGTGCAATTGGTGGAGTCTGGGGGAGGCGTGGTG |
| | encoding F010301647 | CAGCCTGGGGGGTCACTGAGACTCTCCTGTGATGCCT |
| | | CTGGAAGGATACTCCGTATCGGCTACATGAGGTGGCA |
| | | CCGCCAGGGTGCAGGGAAGCAGCGCGAGCTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTGAGGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 305 | Nucleotide sequence | GAGGTGCAATTGGTGGAGTCTGGGGGAGGCGTGGTG |
| | encoding F010301648 | CAGCCTGGGGGGTCACTGAGACTCTCCTGTGATGCCT |
| | | CTGGAAGGATACTCCGTATCGGCTACATGAGGTGGCA |
| | | CCGCCAGGGTGCAGGGAAGCAGCGCGAGTTTGTCGC |
| | | GCGTATTACTGATGATAGTGCTACAGACTATGCAGAC |
| | | TCCGTGAAGGGCCGATTCACCATCTCCAGAGACAACG |
| | | CCAAGAACACGGTGTATCTGCAAATGAACTCCCTGCG |
| | | ACCTGAGGACACGGCCCTCTATTATTGTAACGCGTTG |
| | | GTGACTGCGAGTGTACGTGGTGGGAGTATACATTCTG |
| | | GAACCTATTGGGGCCAGGGGACCCTGGTCACCGTCTC |
| | | CTCA |
| 306 | Nucleotide sequence | GAGGTGCAATTGGTGGAGTCTGGGGGAGGCGTCGTG |
| | encoding F010301649 | CAGCCGGGGGGGTCACTGAGACTCTCCTGTGCGGCCA |
| | | GCGGAAGGATTCTCCGTATCGGCTACATGAGGTGGCA |
| | | CCGCCAGGCGCCGGGGAAGCAGCGCGAGTTTGTCGC |
| | | GCGTATTACTGATGATAGTGCTACAGGTTATGCAGAC |
| | | TCCGTGAAGGGCCGATTCACCATCTCCAGAGACAACG |
| | | CCAAGAACACGGTGTATCTGCAAATGAACAGCCTGCG |
| | | CCCTGAGGACACGGCCCTGTATTATTGTGAGGCGTTG |
| | | GTGACTGCGAGTGTACGTGGTGGGAGTATACATTCTG |
| | | GCACCTATTGGGGCCAGGGGACCCTGGTCACCGTCTC |
| | | CTCA |
| 307 | Nucleotide sequence | GAGGTGCAATTGGTGGAGTCTGGGGGAGGCGTCGTG |
| | encoding F010302307 | CAGCCGGGGGGGTCACTGAGACTCTCCTGTGCGGCCA |
| | | GCGGAAGGATTCTCCGTATCGGCTACATGAGGTGGTA |
| | | TCGCCAGGCGCCGGGGAAGCAGCGCGAGTTTGTCGC |
| | | GCGTATTACTGATGATAGTGCTACAGGTTATGCAGAC |
| | | TCCGTGAAGGGCCGATTCACCATCTCCAGAGACGCGG |
| | | CCAAGAACACGGTGTATCTGCAAATGAACAGCCTGCG |
| | | CCCTGAGGACACGGCCCTGTATTATTGTGAGGCGTTG |
| | | GTGACTGCGAGTGTACGTGGTGGGAGTATACATTCTG |
| | | GCACCTATTGGGGCCAGGGGACCCTGGTCACCGTCTC |
| | | CTCA |
| 308 | Nucleotide sequence | GAGGTGCAATTGGTGGAGTCTGGGGGAGGCGTCGTG |
| | encoding F010302308 | CAGCCGGGGGGGTCACTGAGACTCTCCTGTGCGGCCA |
| | | GCGGAAGGATTCTCCGTATCGGCTACATGAGGTGGTA |
| | | TCGCCAGGCGCCGGGGAAGCAGCGCGAGTTTGTCGC |
| | | GCGTATTACTGATGATAGTGCTACAGGTTATGCAGAC |
| | | TCCGTGAAGGGCCGATTCACCATCTCCAGAGACTATG |
| | | CCAAGAACACGGTGTATCTGCAAATGAACAGCCTGCG |
| | | CCCTGAGGACACGGCCCTGTATTATTGTGAGGCGTTG |
| | | GTGACTGCGAGTGTACGTGGTGGGAGTATACATTCTG |
| | | GCACCTATTGGGGCCAGGGGACCCTGGTCACCGTCTC |
| | | CTCA |
| 309 | Nucleotide sequence | GAGGTGCAATTGGTGGAGTCTGGGGGAGGCGTCGTG |
| | encoding F010302309 | CAGCCGGGGGGGTCACTGAGACTCTCCTGTGCGGCCA |
| | | GCGGAAGGATTCTCCGTATCGGCTACATGAGGTGGTA |
| | | TCGCCAGGCGCCGGGGAAGCAGCGCGAGTTTGTCGC |
| | | GCGTATTACTGATGATAGTGCTACAGGTTATGCAGAC |
| | | TCCGTGAAGGGCCGATTCACCATCTCCAGAGACCAGG |
| | | CCAAGAACACGGTGTATCTGCAAATGAACAGCCTGCG |
| | | CCCTGAGGACACGGCCCTGTATTATTGTGAGGCGTTG |
| | | GTGACTGCGAGTGTACGTGGTGGGAGTATACATTCTG |
| | | GCACCTATTGGGGCCAGGGGACCCTGGTCACCGTCTC |
| | | CTCA |
| 310 | Nucleotide sequence | GAGGTGCAATTGGTGGAGTCTGGTGGTGGAGTTGTTC |
| | encoding F010302391 | AACCCGGTGGTTCTTTGAGATTGTCTTGCGCCGCTTCC |
| | | GGTAGAATCTTGCGTATCGGTTACATGCGTTGGTATA |
| | | GACAAGCTCCCGGTAAGCAAAGAGAGTTCGTCGCCA |
| | | GAATCACCGGAGGTTCTGCTACTGGTTATGCTGATTC |
| | | CGTCAAGGGAAGATTTACCATCTCCAGAGACAACGCT |
| | | AAGAACACTGTTTATTTGCAAATGAACTCCTTGAGAC |
| | | CCGAAGATACCGCTTTGTACTACTGCGAGGCTTTGGT |
| | | CACTGCTTCCGTTAGAGGAGGATCTATCCACTCCGGT |
| | | ACTTACTGGGGTCAAGGAACCCTGGTCACCGTCTCCT |
| | | CA |
| 311 | Nucleotide sequence | GAGGTGCAATTGGTGGAGTCTGGTGGTGGTGTTGTTC |
| | encoding F010302392 | AGCCCGGTGGTTCCTTGAGATTGTCTTGTGCTGCTTCC |
| | | GGTAGAATCTTGAGAATCGGTTACATGAGATGGTACA |
| | | GACAAGCCCCCGGTAAGCAGAGAGAGTTCGTCGCCA |
| | | GAATCACTGGAGGATCTGCTACTGGTTACGCTGACTC |
| | | CGTCAAGGGAAGATTCACCATCTCCAGAGATCAAGCT |
| | | AAGAACACCGTCTACTTGCAGATGAACTCCTTGAGAC |
| | | CAGAGGACACCGCTTTGTACTACTGTGAGGCTTTAGT |
| | | TACTGCTTCCGTTAGAGGTGGTTCCATTCACTCTGGTA |
| | | CTTACTGGGGTCAAGGAACCCTGGTCACCGTCTCCTC |
| | | A |
| 312 | Nucleotide sequence | GAGGTGCAATTGGTGGAGTCTGGGGGAGGAGTGGTG |
| | encoding F010301868 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 313 | Nucleotide sequence | GAGGTGCAATTGGTGGAGTCTGGGGGAGGAGTGGTG |
| | encoding F010301869 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAAACCGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 314 | Nucleotide sequence | GAGGTGCAATTGGTGGAGTCTGGGGGAGGAGTGGTG |
| | encoding F010301870 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCAACTTTT |
| | | CGGGTACAATCAATACTGGCAGAGAGTATCGGTCTGG |
| | | AGACTACTGGGGCCAGGGGACCCTGGTCACCGTCTCC |
| | | TCA |
| 315 | Nucleotide sequence | GAGGTGCAATTGGTGGAGTCTGGGGGAGGAGTGGTG |
| | encoding F010301871 | CAGCCTGGAGGATCACTAAGACTGTCCTGTGCAGCGA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 316 | Nucleotide sequence | GAGGTGCAATTGGTGGAGTCTGGGGGAGGAGTGGTG |
| | encoding F010301872 | CAGCCTGGAGGATCACTAAGACTGTCCTGTGCAACAA |
| | | GCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 317 | Nucleotide sequence | GAGGTGCAATTGGTGGAGTCTGGGGGAGGAGTGGTG |
| | encoding F010301873 | CAGCCTGGAGGATCACTAAGACTGTCCTGTGCAACAA |
| | | CCAGCAGAGCTTTCATCAGGGACGTTTTCACGGGCTG |
| | | GTATCGCCAGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 318 | Nucleotide sequence | GAGGTGCAATTGGTGGAGTCTGGGGGAGGAGTGGTG |
| | encoding F010301874 | CAGCCTGGAGGATCACTAAGACTGTCCTGTGCAACAA |
| | | CCAGCAGAGCTTTCATCAGGGACGTTTTCACGGGCTG |
| | | GTATCGCCGGGCGCCCGGGAAGGAGCGCGAATTGGT |
| | | CGCTCGCATTTACAATGGCGGTAACACAAATTATGCA |
| | | GACTTCGCGAAGGGCCGATTCACCATCTCCAGGGACA |
| | | ACGCCAAGAAGATGGTGTATCTGCAAATGAACAGCCT |
| | | GCGCCCTGAGGACACAGCGCTGTATTACTGCCTTTTTT |
| | | CGGGTACAATCAATACTGGCAGAGAGTATCGGTCTGG |
| | | AGACTACTGGGGCCAGGGGACCCTGGTCACCGTCTCC |
| | | TCA |
| 319 | Nucleotide sequence | GAGGTGCAATTGGTGGAGTCTGGGGGAGGAGTGGTG |
| | encoding F010301875 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACAGCGCGAAGGGCCGATTCACCATCTCCAGGGACA |
| | | ACGCCAAGAAGATGGTGTATCTGCAAATGAACAGCCT |
| | | GCGCCCTGAGGACACAGCGCTGTATTACTGCCTTTTTT |
| | | CGGGTACAATCAATACTGGCAGAGAGTATCGGTCTGG |
| | | AGACTACTGGGGCCAGGGGACCCTGGTCACCGTCTCC |
| | | TCA |
| 320 | Nucleotide sequence | GAGGTGCAATTGGTGGAGTCTGGGGGAGGAGTGGTG |
| | encoding F010301876 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGTGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 321 | Nucleotide sequence | GAGGTGCAATTGGTGGAGTCTGGGGGAGGAGTGGTG |
| | encoding F010301877 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAACATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 322 | Nucleotide sequence | GAGGTGCAATTGGTGGAGTCTGGGGGAGGATTGGTG |
| | encoding F010301892 | CAGCCTGGAGGATCACTAAGACTGTCCTGTGCAACAA |
| | | CCTCTAGAGCTTTCATCAGGGACCTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGAGAATGAGCAATCT |
| | | GAAACCTGAGGACACAGGGGTCTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 323 | Nucleotide sequence | GAGGTGCAATTGGTGGAGTCTGGGGGAGGAGTGGTG |
| | encoding F010301893 | CAGCCTGGAGGATCACTAAGACTGTCCTGTGCAACAA |
| | | CCAGCAGAGCTTTCATCAGGGACGTTTTCACGGGCTG |
| | | GTATCGCCGGGTTCCCGGGAAGCAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGTATCTGCAAATGAACAGCCTG |
| | | CGCCCTGAGGACACAGCGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 324 | Nucleotide sequence | GAGGTGCAATTGGTGGAGTCTGGGGGAGGAGTTGTG |
| | encoding F010301932 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGAGAATGAGCAATCT |
| | | GCGCCCTGAGGACACAGGGCTGTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 325 | Nucleotide sequence | GAGGTGCAATTGGTGGAGTCTGGGGGAGGAGTTGTG |
| | encoding F010301933 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCACCATCTCCAGGGACAA |
| | | CGCCAAGAAGATGGTGACTCTGAGAATGAGCAATCT |
| | | GCGCCCTGAGGACACAGGGCTGTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 326 | Nucleotide sequence | GAGGTGCAATTGGTGGAGTCTGGGGGAGGAGTTGTG |
| | encoding F010301934 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGACCGTGACTCTGAGAATGAGCAATCT |
| | | GCGCCCTGAGGACACAGGGCTGTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 327 | Nucleotide sequence | GAGGTGCAATTGGTGGAGTCTGGGGGAGGAGTTGTG |
| | encoding F010301935 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGTATCTGAGAATGAGCAATCTG |
| | | CGCCCTGAGGACACAGGGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 328 | Nucleotide sequence | GAGGTGCAATTGGTGGAGTCTGGGGGAGGAGTTGTG |
| | encoding F010301936 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGCAAATGAGCAATCTG |
| | | CGCCCTGAGGACACAGGGCTGTATTACTGCCTTTTTTC |
| | | GGGTACAATCAATACTGGCAGAGAGTATCGGTCTGGA |
| | | GACTACTGGGGCCAGGGGACCCTGGTCACCGTCTCCT |
| | | CA |
| 329 | Nucleotide sequence | GAGGTGCAATTGGTGGAGTCTGGGGGAGGAGTTGTG |
| | encoding F010301937 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGAGAATGAACAATCT |
| | | GCGCCCTGAGGACACAGGGCTGTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 330 | Nucleotide sequence | GAGGTGCAATTGGTGGAGTCTGGGGGAGGAGTTGTG |
| | encoding F010301938 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGAGAATGAGCAGCCT |
| | | GCGCCCTGAGGACACAGGGCTGTATTACTGCCTTTTT |
| | | TCGGGTACAATCAATACTGGCAGAGAGTATCGGTCTG |
| | | GAGACTACTGGGGCCAGGGGACCCTGGTCACCGTCTC |
| | | CTCA |
| 331 | Nucleotide sequence | GAGGTGCAATTGGTGGAGTCTGGGGGAGGAGTTGTG |
| | encoding F010301939 | CAGCCTGGAGGATCACTAAGACTGTCCTGTGCAACAA |
| | | CCTCCAGAGCTTTCATCAGGGACGTTTTCACGGGCTG |
| | | GTATCGCCGGGTTCCCGGGAAGGAGCGCGAATTGGTC |
| | | GCTCGCATTTACAATGGCGGTAACACAAATTATGCAG |
| | | ACTTCGCGAAGGGCCGATTCTCCATCTCCAGGGACAA |
| | | CGCCAAGAAGATGGTGACTCTGAGAATGAGCAATCT |
| | | GCGCCCTGAGGACACAGCGCTGTATTACTGCCTTTTTT |
| | | CGGGTACAATCAATACTGGCAGAGAGTATCGGTCTGG |
| | | AGACTACTGGGGCCAGGGGACCCTGGTCACCGTCTCC |
| | | TCA |
| 332 | Nucleotide sequence | GAGGTGCAATTGGTGGAGTCTGGGGGAGGAGTGGTG |
| | encoding F010302333 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 333 | Nucleotide sequence | GAGGTGCAATTGGTGGAGTCTGGGGGAGGAGTGGTG |
| | encoding F010302334 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 334 | Nucleotide sequence | GAGGTGCAATTGGTGGAGTCTGGGGGAGGAGTGGTG |
| | encoding F010302335 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACGAAAGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 335 | Nucleotide sequence | GAGGTGCAATTGGTGGAGTCTGGGGGAGGAGTGGTG |
| | encoding F010302336 | CAGCCTGGAGGATCACTAAGACTGTCCTGTGCAGCGA |
| | | GCCATAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 336 | Nucleotide sequence | GAGGTGCAATTGGTGGAGTCTGGGGGAGGAGTGGTG |
| | encoding F010302337 | CAGCCTGGAGGATCACTAAGACTGTCCTGTGCAGCGA |
| | | GCCATAGACAGTTCATCAGGGACGTTTTCACGGGCTG |
| | | GTATCGCCGGGCGCCCGGGAAGCAGCGCGAATTGGT |
| | | CGCTCGCATTTACGAAGGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 337 | Nucleotide sequence | GAGGTGCAATTGGTGGAGTCTGGGGGAGGAGTGGTG |
| | encoding F010302338 | CAGCCTGGAGGATCACTAAGACTGTCCTGTGCAGCGA |
| | | GCCATAGAGCTTTCATCAGGGACGTTTTCACGGGCTG |
| | | GTATCGCCGGGCGCCCGGGAAGCAGCGCGAATTGGT |
| | | CGCTCGCATTTACGAAAGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 338 | Nucleotide sequence | GAGGTGCAATTGGTGGAGTCTGGGGGAGGAGTGGTG |
| | encoding F010302339 | CAGCCTGGAGGATCACTAAGACTGTCCTGTGCAGCGA |
| | | GCCATAGAGCTTTCATCAGGGACGTTTTCACGGGCTG |
| | | GTATCGCCGGGCGCCCGGGAAGCAGCGCGAATTGGT |
| | | CGCTCGCATTTACGAAGGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 339 | Nucleotide sequence | GAGGTGCAATTGGTGGAGTCTGGGGGAGGAGTGGTG |
| | encoding F010302340 | CAGCCTGGAGGATCACTAAGACTGTCCTGTGCAGCGA |
| | | GCCATAGAGCTTTCATCAGGGACCTGTTCACGGGCTG |
| | | GTATCGCCGGGCGCCCGGGAAGCAGCGCGAATTGGT |
| | | CGCTCGCATTTACGAAAGCGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 340 | Nucleotide sequence | GAGGTGCAATTGGTGGAGTCTGGGGGAGGAGTGGTG |
| | encoding F010302341 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 341 | Nucleotide sequence | GAGGTGCAATTGGTGGAGTCTGGGGGAGGAGTGGTG |
| | encoding F010302342 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 342 | Nucleotide sequence | GAGGTGCAATTGGTGGAGTCTGGGGGAGGAGTGGTG |
| | encoding F010302343 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACGAAAGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 343 | Nucleotide sequence | GAGGTGCAATTGGTGGAGTCTGGGGGAGGAGTGGTG |
| | encoding F010302344 | CAGCCTGGAGGATCACTAAGACTGTCCTGTGCAGCGA |
| | | GCCATAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 344 | Nucleotide sequence | GAGGTGCAATTGGTGGAGTCTGGGGGAGGAGTGGTG |
| | encoding F010302345 | CAGCCTGGAGGATCACTAAGACTGTCCTGTGCAGCGA |
| | | GCCATAGACAGTTCATCAGGGACGTTTTCACGGGCTG |
| | | GTATCGCCAGGCGCCCGGGAAGCAGCGCGAATTGGT |
| | | CGCTCGCATTTACGAAGGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 345 | Nucleotide sequence | GAGGTGCAATTGGTGGAGTCTGGGGGAGGAGTGGTG |
| | encoding F010302346 | CAGCCTGGAGGATCACTAAGACTGTCCTGTGCAGCGA |
| | | GCCATAGAGCTTTCATCAGGGACGTTTTCACGGGCTG |
| | | GTATCGCCAGGCGCCCGGGAAGCAGCGCGAATTGGT |
| | | CGCTCGCATTTACGAAAGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 346 | Nucleotide sequence | GAGGTGCAATTGGTGGAGTCTGGGGGAGGAGTGGTG |
| | encoding F010302347 | CAGCCTGGAGGATCACTAAGACTGTCCTGTGCAGCGA |
| | | GCCATAGAGCTTTCATCAGGGACGTTTTCACGGGCTG |
| | | GTATCGCCAGGCGCCCGGGAAGCAGCGCGAATTGGT |
| | | CGCTCGCATTTACGAAGGCGGTAACACACAGTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 347 | Nucleotide sequence | GAGGTGCAATTGGTGGAGTCTGGGGGAGGAGTGGTG |
| | encoding F010302348 | CAGCCTGGAGGATCACTAAGACTGTCCTGTGCAGCGA |
| | | GCCATAGAGCTTTCATCAGGGACCTGTTCACGGGCTG |
| | | GTATCGCCAGGCGCCCGGGAAGCAGCGCGAATTGGT |
| | | CGCTCGCATTTACGAAAGCGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 348 | Nucleotide sequence | GAGGTGCAATTGGTGGAGTCTGGGGGAGGAGTGGTG |
| | encoding F010302349 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACAAATTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 349 | Nucleotide sequence | GAGGTGCAATTGGTGGAGTCTGGGGGAGGAGTGGTG |
| | encoding F010302350 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 350 | Nucleotide sequence | GAGGTGCAATTGGTGGAGTCTGGGGGAGGAGTGGTG |
| | encoding F010302351 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACGAAAGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 351 | Nucleotide sequence | GAGGTGCAATTGGTGGAGTCTGGGGGAGGAGTGGTG |
| | encoding F010302352 | CAGCCTGGAGGATCACTAAGACTGTCCTGTGCAGCGA |
| | | GCCATAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 352 | Nucleotide sequence | GAGGTGCAATTGGTGGAGTCTGGGGGAGGAGTGGTG |
| | encoding F010302353 | CAGCCTGGAGGATCACTAAGACTGTCCTGTGCAGCGA |
| | | GCCATAGACAGTTCATCAGGGACGTTTTCACGGGCTG |
| | | GTATCGCCGGGCGCCCGGGAAGCAGCGCGAATTGGT |
| | | CGCTCGCATTTACGAAGGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 353 | Nucleotide sequence | GAGGTGCAATTGGTGGAGTCTGGGGGAGGAGTGGTG |
| | encoding F010302354 | CAGCCTGGAGGATCACTAAGACTGTCCTGTGCAGCGA |
| | | GCCATAGAGCTTTCATCAGGGACGTTTTCACGGGCTG |
| | | GTATCGCCGGGCGCCCGGGAAGCAGCGCGAATTGGT |
| | | CGCTCGCATTTACGAAAGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 354 | Nucleotide sequence | GAGGTGCAATTGGTGGAGTCTGGGGGAGGAGTGGTG |
| | encoding F010302355 | CAGCCTGGAGGATCACTAAGACTGTCCTGTGCAGCGA |
| | | GCCATAGAGCTTTCATCAGGGACGTTTTCACGGGCTG |
| | | GTATCGCCGGGCGCCCGGGAAGCAGCGCGAATTGGT |
| | | CGCTCGCATTTACGAAGGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 355 | Nucleotide sequence | GAGGTGCAATTGGTGGAGTCTGGGGGAGGAGTGGTG |
| | encoding F010302356 | CAGCCTGGAGGATCACTAAGACTGTCCTGTGCAGCGA |
| | | GCCATAGAGCTTTCATCAGGGACCTGTTCACGGGCTG |
| | | GTATCGCCGGGCGCCCGGGAAGCAGCGCGAATTGGT |
| | | CGCTCGCATTTACGAAAGCGGTAACACAAATTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 356 | Nucleotide sequence | GAGGTGCAATTGGTGGAGTCTGGGGGAGGAGTGGTG |
| | encoding F010302357 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACAAATTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 357 | Nucleotide sequence | GAGGTGCAATTGGTGGAGTCTGGGGGAGGAGTGGTG |
| | encoding F010302358 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 358 | Nucleotide sequence | GAGGTGCAATTGGTGGAGTCTGGGGGAGGAGTGGTG |
| | encoding F010302359 | CAGCCTGGAGGATCACTAAGACTGTCCTGTGCAGCGA |
| | | GCAGCAGACAGTTCATCAGGGACGTTTTCACGGGCTG |
| | | GTATCGCCAGGCGCCCGGGAAGCAGCGCGAATTGGT |
| | | CGCTCGCATTTACGAAAGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 359 | Nucleotide sequence | GAGGTGCAATTGGTGGAGTCTGGGGGAGGAGTGGTG |
| | encoding F010302360 | CAGCCTGGAGGATCACTAAGACTGTCCTGTGCAGCGA |
| | | GCCATAGACAGTTCATCAGGGACGTTTTCACGGGCTG |
| | | GTATCGCCAGGCGCCCGGGAAGCAGCGCGAATTGGT |
| | | CGCTCGCATTTACAATGAAGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 360 | Nucleotide sequence | GAGGTGCAATTGGTGGAGTCTGGGGGAGGAGTGGTG |
| | encoding F010302361 | CAGCCTGGAGGATCACTAAGACTGTCCTGTGCAGCGA |
| | | GCCATAGACAGTTCATCAGGGACGTTTTCACGGGCTG |
| | | GTATCGCCAGGCGCCCGGGAAGCAGCGCGAATTGGT |
| | | CGCTCGCATTTACGAAGGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 361 | Nucleotide sequence | GAGGTGCAATTGGTGGAGTCTGGGGGAGGAGTGGTG |
| | encoding F010302362 | CAGCCTGGAGGATCACTAAGACTGTCCTGTGCAGCGA |
| | | GCCATAGAGCTTTCATCAGGGACGTTTTCACGGGCTG |
| | | GTATCGCCAGGCGCCCGGGAAGCAGCGCGAATTGGT |
| | | CGCTCGCATTTACGAAAGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 362 | Nucleotide sequence | GAGGTGCAATTGGTGGAGTCTGGGGGAGGAGTGGTG |
| | encoding F010302363 | CAGCCTGGAGGATCACTAAGACTGTCCTGTGCAGCGA |
| | | GCCATAGAGCTTTCATCAGGGACGTTTTCACGGGCTG |
| | | GTATCGCCAGGCGCCCGGGAAGCAGCGCGAATTGGT |
| | | CGCTCGCATTTACGAAGGCGGTAACACACAGTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 363 | Nucleotide sequence | GAGGTGCAATTGGTGGAGTCTGGGGGAGGAGTGGTG |
| | encoding F010302364 | CAGCCTGGAGGATCACTAAGACTGTCCTGTGCAGCGA |
| | | GCCATAGAGCTTTCATCAGGGACCTGTTCACGGGCTG |
| | | GTATCGCCAGGCGCCCGGGAAGCAGCGCGAATTGGT |
| | | CGCTCGCATTTACGAAAGCGGTAACACAAATTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 364 | Nucleotide sequence | GAGGTGCAATTGGTGGAGTCTGGGGGAGGAGTGGTG |
| | encoding F010302365 | CAGCCTGGAGGATCACTAAGACTGTCCTGTGCAGCGA |
| | | GCAGCAGAGCTTTCATCAGGGACGTTTTCACGGGCTG |
| | | GTATCGCCGGGCGCCCGGGAAGCAGCGCGAATTGGT |
| | | CGCTCGCATTTACAATGGCGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 365 | Nucleotide sequence | GAGGTGCAATTGGTGGAGTCTGGGGGAGGAGTGGTG |
| | encoding F010302366 | CAGCCTGGAGGATCACTAAGACTGTCCTGTGCAGCGA |
| | | GCAGCAGAGCTTTCATCAGGGACGTTTTCACGGGCTG |
| | | GTATCGCCAGGCGCCCGGGAAGCAGCGCGAATTGGT |
| | | CGCTCGCATTTACAATGGCGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 366 | Nucleotide sequence | GAGGTGCAATTGGTGGAGTCTGGGGGAGGAGTGGTG |
| | encoding F010302367 | CAGCCTGGAGGATCACTAAGACTGTCCTGTGCAGCGA |
| | | GCAGCAGAGCTTTCATCAGGGACGTTTTCACGGGCTG |
| | | GTATCGCCAGGCGCCCGGGAAGCAGCGCGAATTGGT |
| | | CGCTCGCATTTACAATGGCGGTAACACAAATTATGCA |
| | | GACAGCGTGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 367 | Nucleotide sequence | GAGGTGCAATTGGTGGAGTCTGGGGGAGGAGTGGTG |
| | encoding F010302368 | CAGCCTGGAGGATCACTAAGACTGTCCTGTGCAGCGA |
| | | GCAGCAGACAGTTCATCAGGGACGTTTTCACGGGCTG |
| | | GTATCGCCGGGCGCCCGGGAAGCAGCGCGAATTGGT |
| | | CGCTCGCATTTACAATGAAGGTAACACAAATTATGCA |
| | | GACAGCGCGAAGGGCCGATTCACCATCTCCAGGGAC |
| | | AACGCCAAGAAAACCGTGTATCTGCAAATGAACAGC |
| | | CTGCGCCCTGAGGACACAGCGCTGTATTACTGCCTTT |
| | | TTTCGGGTACAATCAGCACTGGCAGAGAGTATCGGTC |
| | | TGGAGACTACTGGGGCCAGGGGACCCTGGTCACCGTC |
| | | TCCTCA |
| 368 | Nucleotide sequence | GAGGTGCAATTGGTGGAGTCTGGGGGAGGCTTGGCG |
| | encoding F010301656 | CAGCCTGGGGGGTCTCTGAGACTCTCCTGTGCAGCCT |
| | | CTGGACCCGTCTTTAATATCAACCGCATGGCCTGGTA |
| | | TCGCCGGGCTCCAGGGAAGCAGCGCGAATTGGTCGC |
| | | ATATGTCACCCCTACTGGTGATATAAGTTATACTGAC |
| | | TCCGTGAAGGGCCGATTCACCATTTCTAGGGACGGCT |
| | | CCAAGCGGTGGTCTCTACAAATGAACAGCCTGACACC |
| | | TGAGGACACGGCCGTCTATTACTGTCGCGCTTTACTA |
| | | CAACCGGATAGTTATTCTAATACGCGCACATATTGGG |
| | | GCCAGGGGACCCTGGTCACCGTCTCCTCA |
| 369 | Nucleotide sequence | GAGGTGCAATTGGTGGAGTCTGGGGGAGGCGTGGTTC |
| | encoding F010301840 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACGGCTCC |
| | | AAGCGGACCTGGTCTCTACAAATGAACAGCCTGCGCC |
| | | CTGAGGACACGGCCCTGTATTACTGTCGCGCTTTACT |
| | | ACAACCGGATAGTTATTCTAATACGCGCACATATTGG |
| | | GGCCAGGGGACCCTGGTCACCGTCTCCTCA |
| 370 | Nucleotide sequence | GAGGTGCAATTGGTGGAGTCTGGGGGAGGCGTGGTTC |
| | encoding F010301841 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACGGCTCC |
| | | AAGCGGTGGTCTCTACAAATGAACAGCCTGCGCCCTG |
| | | AGGACACGGCCCTGTATTACTGTCGCGCTTTACTACA |
| | | ACCGGATAGTTATTCTAATACGCGCACATATTGGGGC |
| | | CAGGGGACCCTGGTCACCGTCTCCTCA |
| 371 | Nucleotide sequence | GAGGTGCAATTGGTGGAGTCTGGGGGAGGCGTGGTTC |
| | encoding F010301842 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGTGGTCTCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 372 | Nucleotide sequence | GAGGTGCAATTGGTGGAGTCTGGGGGAGGCGTGGTTC |
| | encoding F010301843 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACAACTCC |
| | | AAGCGGTGGTCTCTACAAATGAACAGCCTGCGCCCTG |
| | | AGGACACGGCCCTGTATTACTGTCGCGCTTTACTACA |
| | | ACCGGATAGTTATTCTAATACGCGCACATATTGGGGC |
| | | CAGGGGACCCTGGTCACCGTCTCCTCA |
| 373 | Nucleotide sequence | GAGGTGCAATTGGTGGAGTCTGGGGGAGGCGTGGTTC |
| | encoding F010301844 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACGGCTCC |
| | | AAGAACTGGTCTCTACAAATGAACAGCCTGCGCCCTG |
| | | AGGACACGGCCCTGTATTACTGTCGCGCTTTACTACA |
| | | ACCGGATAGTTATTCTAATACGCGCACATATTGGGGC |
| | | CAGGGGACCCTGGTCACCGTCTCCTCA |
| 374 | Nucleotide sequence | GAGGTGCAATTGGTGGAGTCTGGGGGAGGCGTGGTTC |
| | encoding F010301845 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACGGCTCC |
| | | AAGCGGGTCTCTCTACAAATGAACAGCCTGCGCCCTG |
| | | AGGACACGGCCCTGTATTACTGTCGCGCTTTACTACA |
| | | ACCGGATAGTTATTCTAATACGCGCACATATTGGGGC |
| | | CAGGGGACCCTGGTCACCGTCTCCTCA |
| 375 | Nucleotide sequence | GAGGTGCAATTGGTGGAGTCTGGGGGAGGCGTGGTTC |
| | encoding F010301846 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATACTGACTC |
| | | CGTGAAGGGCCGATTCACCATTTCTCGCGACGGCTCC |
| | | AAGCGGTGGTATCTACAAATGAACAGCCTGCGCCCTG |
| | | AGGACACGGCCCTGTATTACTGTCGCGCTTTACTACA |
| | | ACCGGATAGTTATTCTAATACGCGCACATATTGGGGC |
| | | CAGGGGACCCTGGTCACCGTCTCCTCA |
| 376 | Nucleotide sequence | GAGGTGCAATTGGTGGAGTCTGGGGGAGGCGTGGTTC |
| | encoding F010301847 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACAACTC |
| | | CAAGAACGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 377 | Nucleotide sequence | GAGGTGCAATTGGTGGAGTCTGGGGGAGGCGTGGTTC |
| | encoding F010301848 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACAACTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 378 | Nucleotide sequence | GAGGTGCAATTGGTGGAGTCTGGGGGAGGCGTGGTTC |
| | encoding F010301865 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 379 | Nucleotide sequence | GAGGTGCAATTGGTGGAGTCTGGGGGAGGCGTGGTTC |
| | encoding F010301866 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGAACGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 380 | Nucleotide sequence | GAGGTGCAATTGGTGGAGTCTGGGGGAGGCGTGGTTC |
| | encoding F010302310 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTAATACGCGCACATATTGGGG |
| | | CCAGGGGACCCTGGTCACCGTCTCCTCA |
| 381 | Nucleotide sequence | GAGGTGCAATTGGTGGAGTCTGGGGGAGGCGTGGTTC |
| | encoding F010302311 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTAATACGCGCACATATTGGGG |
| | | CCAGGGGACCCTGGTCACCGTCTCCTCA |
| 382 | Nucleotide sequence | GAGGTGCAATTGGTGGAGTCTGGGGGAGGCGTGGTTC |
| | encoding F010302312 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTATTACGCGCACATATTGGGG |
| | | CCAGGGGACCCTGGTCACCGTCTCCTCA |
| 383 | Nucleotide sequence | GAGGTGCAATTGGTGGAGTCTGGGGGAGGCGTGGTTC |
| | encoding F010302313 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCC |
| | | GAGGACACGGCCCTGTATTACTGTCGCGCTTTACTAC |
| | | AACCGGATAGTTATTCTATTACGCGCACATATTGGGG |
| | | CCAGGGGACCCTGGTCACCGTCTCCTCA |
| 384 | Nucleotide sequence | GAGGTGCAATTGGTGGAGTCTGGGGGAGGCGTGGTTC |
| | encoding F010302314 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTATTACGCGCACATATTGGGG |
| | | CCAGGGGACCCTGGTCACCGTCTCCTCA |
| 385 | Nucleotide sequence | GAGGTGCAATTGGTGGAGTCTGGGGGAGGCGTGGTTC |
| | encoding F010302315 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTATTACGCGCACATATTGGGG |
| | | CCAGGGGACCCTGGTCACCGTCTCCTCA |
| 386 | Nucleotide sequence | GAGGTGCAATTGGTGGAGTCTGGGGGAGGCGTGGTTC |
| | encoding F010302316 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTATTACGCGCACATATTGGGG |
| | | CCAGGGGACCCTGGTCACCGTCTCCTCA |
| 387 | Nucleotide sequence | GAGGTGCAATTGGTGGAGTCTGGGGGAGGCGTGGTTC |
| | encoding F010302317 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTATTACGCGCACATATTGGGG |
| | | CCAGGGGACCCTGGTCACCGTCTCCTCA |
| 388 | Nucleotide sequence | GAGGTGCAATTGGTGGAGTCTGGGGGAGGCGTGGTTC |
| | encoding F010302318 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTATTACGCGCACATATTGGGG |
| | | CCAGGGGACCCTGGTCACCGTCTCCTCA |
| 389 | Nucleotide sequence | GAGGTGCAATTGGTGGAGTCTGGGGGAGGCGTGGTTC |
| | encoding F010302319 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTATTACGCGCACATATTGGGG |
| | | CCAGGGGACCCTGGTCACCGTCTCCTCA |
| 390 | Nucleotide sequence | GAGGTGCAATTGGTGGAGTCTGGGGGAGGCGTGGTTC |
| | encoding F010302320 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 391 | Nucleotide sequence | GAGGTGCAATTGGTGGAGTCTGGGGGAGGCGTGGTTC |
| | encoding F010302321 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTATTACGCGCACATATTGGGG |
| | | CCAGGGGACCCTGGTCACCGTCTCCTCA |
| 392 | Nucleotide sequence | GAGGTGCAATTGGTGGAGTCTGGGGGAGGCGTGGTTC |
| | encoding F010302322 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTATTACGCGCACATATTGGGG |
| | | CCAGGGGACCCTGGTCACCGTCTCCTCA |
| 393 | Nucleotide sequence | GAGGTGCAATTGGTGGAGTCTGGGGGAGGCGTGGTTC |
| | encoding F010302323 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTATTACGCGCACATATTGGGG |
| | | CCAGGGGACCCTGGTCACCGTCTCCTCA |
| 394 | Nucleotide sequence | GAGGTGCAATTGGTGGAGTCTGGGGGAGGCGTGGTTC |
| | encoding F010302324 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTATTACGCGCACATATTGGGG |
| | | CCAGGGGACCCTGGTCACCGTCTCCTCA |
| 395 | Nucleotide sequence | GAGGTGCAATTGGTGGAGTCTGGGGGAGGCGTGGTTC |
| | encoding F010302325 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATAGTTATTCTAATACGCGCACATATTGGGG |
| | | CCAGGGGACCCTGGTCACCGTCTCCTCA |
| 396 | Nucleotide sequence | GAGGTGCAATTGGTGGAGTCTGGGGGAGGCGTGGTTC |
| | encoding F010302326 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTAATACGCGCACATATTGGGG |
| | | CCAGGGGACCCTGGTCACCGTCTCCTCA |
| 397 | Nucleotide sequence | GAGGTGCAATTGGTGGAGTCTGGGGGAGGCGTGGTTC |
| | encoding F010302327 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTAATACGCGCACATATTGGGG |
| | | CCAGGGGACCCTGGTCACCGTCTCCTCA |
| 398 | Nucleotide sequence | GAGGTGCAATTGGTGGAGTCTGGGGGAGGCGTGGTTC |
| | encoding F010302328 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCAGTTATTCTAATACGCGCACATATTGGGG |
| | | CCAGGGGACCCTGGTCACCGTCTCCTCA |
| 399 | Nucleotide sequence | GAGGTGCAATTGGTGGAGTCTGGGGGAGGCGTGGTTC |
| | encoding F010302329 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTAATACGCGCACATATTGGGG |
| | | CCAGGGGACCCTGGTCACCGTCTCCTCA |
| 400 | Nucleotide sequence | GAGGTGCAATTGGTGGAGTCTGGGGGAGGCGTGGTTC |
| | encoding F010302330 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGCGTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTAATACGCGCACATATTGGGG |
| | | CCAGGGGACCCTGGTCACCGTCTCCTCA |
| 401 | Nucleotide sequence | GAGGTGCAATTGGTGGAGTCTGGGGGAGGCGTGGTTC |
| | encoding F010302331 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATCGCTATTCTAATACGCGCACATATTGGGG |
| | | CCAGGGGACCCTGGTCACCGTCTCCTCA |
| 402 | Nucleotide sequence | GAGGTGCAATTGGTGGAGTCTGGGGGAGGCGTGGTTC |
| | encoding F010302332 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACGGCTC |
| | | CAAGCGGGTGTATCTACAAATGAACAGCCTGCGCCCT |
| | | GAGGACACGGCCCTGTATTACTGTCGCGCTTTACTAC |
| | | AACCGCGCCGCTATTCTAATACGCGCACATATTGGGG |
| | | CCAGGGGACCCTGGTCACCGTCTCCTCA |
| 403 | Nucleotide sequence | GAGGTGCAATTGGTGGAGTCTGGGGGAGGCGTGGTTC |
| | encoding F010302370 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGAGCAGTTATTCTATTACGCGCACATATTGGGG |
| | | CCAGGGGACCCTGGTCACCGTCTCCTCA |
| 404 | Nucleotide sequence | GAGGTGCAATTGGTGGAGTCTGGGGGAGGCGTGGTTC |
| | encoding F010302371 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGAACGTGTATTCTATTACGCGCACATATTGGGG |
| | | CCAGGGGACCCTGGTCACCGTCTCCTCA |
| 405 | Nucleotide sequence | GAGGTGCAATTGGTGGAGTCTGGGGGAGGCGTGGTTC |
| | encoding F010302372 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGACCGCTC |
| | | CAAGCGGGTGTATCTACAAATGAACAGCCTGCGCCCT |
| | | GAGGACACGGCCCTGTATTACTGTCGCGCTTTACTAC |
| | | AACCGGATGTGTATTCTATTACGCGCACATATTGGGG |
| | | CCAGGGGACCCTGGTCACCGTCTCCTCA |
| 406 | Nucleotide sequence | GAGGTGCAATTGGTGGAGTCTGGGGGAGGCGTGGTTC |
| | encoding F010302383 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGGTGGCTC |
| | | CAAGCGGGTGTATCTACAAATGAACAGCCTGCGCCCT |
| | | GAGGACACGGCCCTGTATTACTGTCGCGCTTTACTAC |
| | | AACCGAGCAGTTATTCTGGCACGCGCACATATTGGGG |
| | | CCAGGGGACCCTGGTCACCGTCTCCTCA |
| 407 | Nucleotide sequence | GAGGTGCAATTGGTGGAGTCTGGGGGAGGCGTGGTTC |
| | encoding F010302384 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCGGTGGCTC |
| | | CAAGCGGGTGTATCTACAAATGAACAGCCTGCGCCCT |
| | | GAGGACACGGCCCTGTATTACTGTCGCGCTTTACTAC |
| | | AACCGAGCAGTTATTCTATTACGCGCACATATTGGGG |
| | | CCAGGGGACCCTGGTCACCGTCTCCTCA |
| 408 | Nucleotide sequence | GAGGTGCAATTGGTGGAGTCTGGGGGAGGCGTGGTTC |
| | encoding F010302385 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCCAGGGCTC |
| | | CAAGCGGGTGTATCTACAAATGAACAGCCTGCGCCCT |
| | | GAGGACACGGCCCTGTATTACTGTCGCGCTTTACTAC |
| | | AACCGAGCAGTTATTCTGGCACGCGCACATATTGGGG |
| | | CCAGGGGACCCTGGTCACCGTCTCCTCA |
| 409 | Nucleotide sequence | GAGGTGCAATTGGTGGAGTCTGGGGGAGGCGTGGTTC |
| | encoding F010302386 | AGCCTGGGGGGTCTCTGAGACTCTCCTGTGCAGCCTC |
| | | TGGACCCGTGTTCAATATCAACCGCATGGCCTGGTAT |
| | | CGCCAGGCTCCAGGGAAGCAGCGCGAATTGGTCGCA |
| | | TATGTCACCCCTACTGGTGATATAAGTTATGCGGACT |
| | | CCGTGAAGGGCCGATTCACCATTTCTCGCCAGGGCTC |
| | | CAAGCGGGTGTATCTACAAATGAACAGCCTGCGCCCT |
| | | GAGGACACGGCCCTGTATTACTGTCGCGCTTTACTAC |
| | | AACCGAGCAGTTATTCTATTACGCGCACATATTGGGG |
| | | CCAGGGGACCCTGGTCACCGTCTCCTCA |
| 410 | F0103240B04 (No tag) | EVQLVESGGGLVQAGGSLRLSCAASGGTGRRYAMGWF |
| | | RQAPGKEREIVAAIRWSAMTYYADDGKGRFTISRDNAK |
| | | NTVYLQMNSLKPEDTAIYYCAYTWDYFKYDQVRAYRG |
| | | WGQGTLVTVSS |
| 411 | F0103478E09 (No tag) | EVQLVESGGGLVQAGGSLRLSCAASGRAFSTLAMGWF |
| | | RQAPGKEREFVAAISRNGNNSATGDSLKGRFTISRDSTK |
| | | STVF |
| | | LQMNTLKPEDTAVYYCAAISTPSASHPYVRKESYRYWG |
| | | QGTLVTVSS |
| 412 | F0103492E09 (No tag) | EVQLVESGGGLVQAGGSLRLSCAASKSILSFAYMRWYR |
| | | QAPGKQREFVASIAIGGATSYTDSVKGRFTISRDNAKNT |
| | | VYLQMNSLKPEDTAVYYCSAPAGQYRGQGTLVTVSS |
| 413 | F0103500E03 (No tag) | EVQLVESGGGLVQPGGSLRLSCAASGRTFSRYQMGWFR |
| | | QAPGKEREFVAYISWSGSTRYVDSVKGRFTISRDNAKNT |
| | | VYLQMNSLKPEDTAVYHCAAGTAGIISSRPETYDSWGQ |
| | | GTLVTVSS |
| 414 | F0103505D08 (No tag) | EVQLVESGGGLVQAGGSLRLSCVTSGRTSDLSTMNWFR |
| | | QAPGKEREFVARITRRGSTYYAESVKERFIISRDNAKNT |
| | | VYL |
| | | QMNSLKPEDTANYYCTAASEMGYHYRGQGTLVTVSS |
| 415 | F0103495F09 (No tag) | EVQLVESGGGLVQAGSSLSLSCAASGRALSTYAMGWFR |
| | | QAPGKEREFVARISRSGITTYYTDSVKGRFTISRDRAKDT |
| | | VY |
| | | LQMNSLKPEDTAIYLCAADASTNPAGYYLRNRYDYWG |
| | | QGTLVTVSS |
| 416 | F0103240B04-CDR1 | GGTGRRYAMGW |
| 417 | F0103240B04-CDR2 | AIRWSAMTY |
| 418 | F0103240B04-CDR3 | TWDYFKYDQVRAYRG |
| 419 | F0103478E09-CDR1 | GRAFSTLAMG |
| 420 | F0103478E09-CDR2 | ISRNGNNS |
| 421 | F0103478E09-CDR3 | ISTPSASHPYVRKESYRY |
| 422 | F0103492E09-CDR1 | KSILSFAYMR |
| 423 | F0103492E09-CDR2 | SIAIGGATS |
| 424 | F0103492E09-CDR3 | PAGQYR |
| 425 | F0103500E03-CDR1 | GRTFSRYQMG |
| 426 | F0103500E03-CDR2 | YISWSGSTR |
| 427 | F0103500E03-CDR3 | GTAGIISSRPETYDS |
| 428 | F0103505D08-CDR1 | GRTSDLSTMN |
| 429 | F0103505D08-CDR2 | RITRRGSTY |
| 430 | F0103505D08-CDR3 | ASEMGYHYR |
| 431 | F0103495F09-CDR1 | GRALSTYAMG |
| 432 | F0103495F09-CDR2 | RISRSGITT |
| 433 | F0103495F09-CDR3 | DASTNPAGYYLRNRYDY |
| 434 | F103275B05(N93R) | EVQLVESGGGLVQPGGSLRLSCAASGSIFNINSMA |
| | | WYRRAPGKQRELVASSTNGGSTNYADSVKGRFTIS |
| | | RDNAKRVYLQMNSLTPEDTAVYYCRALLQPSIYDI |
| | | SRTYWGQGTLVTVSS |
| 435 | F0103275B05 | DVQLVESGGGLVQPGGSLRLSCAASGSIFNINSMA |
| | (E1D, N93R) | WYRRAPGKQRELVASSTNGGSTNYADSVKGRFTIS |
| | | RDNAKRVYLQMNSLTPEDTAVYYCRALLQPSIYDI |
| | | SRTYWGQGTLVTVSS |
| 436 | F0103478E09 (L108Q) | EVQLVESGGGLVQAGGSLRLSCAASGRAFSTLAMGWF |
| | | RQAPGKEREFVAAISRNGNNSATGDSLKGRFTISRDSTK |
| | | STVFLQMNTLKPEDTAVYYCAAISTPSASHPYVRKESYR |
| | | YWGQGTQVTVSS |
| 437 | F0103505D08 (L108Q) | EVQLVESGGGLVQAGGSLRLSCVTSGRTSDLSTMNWFR |
| | | QAPGKEREFVARITRRGSTYYAESVKERFIISRDNAKNT |
| | | VYLQMNSLKPEDTANYYCTAASEMGYHYRGQGTQVT |
| | | VSS |
| 438 | F0103500E03 | EVQLVESGGGLVQAGGSLRLSCAASGRTFSRYQMGWF |
| | (P14A, L108Q) | RQAPGKEREFVAYISWSGSTRYVDSVKGRFTISRDNAK |
| | | NTVYLQMNSLKPEDTAVYHCAAGTAGIISSRPETYDSW |
| | | GQGTQVTVSS |
| 439 | F010302375:F0103275B05 | DVQLVESGGGLVQPGGSLRLSCAASGSIFNINSMAWYR |
| | (E1D, N93R)-50GS- | RAPGKQRELVASSTNGGSTNYADSVKGRFTISRDNAKR |
| | F0103478E09(L108Q)- | VYLQMNSLTPEDTAVYYCRALLQPSIYDISRTYWGQGT |
| | FLAG3-HIS6 | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASGRAFSTLAMGWFRQAPGKEREFVAAISRNGN |
| | | NSATGDSLKGRFTISRDSTKSTVFLQMNTLKPEDTAVYY |
| | | CAAISTPSASHPYVRKESYRYWGQGTQVTVSSGAADYK |
| | | DHDGDYKDHDIDYKDDDDKGAAHHHHHH |
| 440 | F010302377:F0103275B05 | DVQLVESGGGLVQPGGSLRLSCAASGSIFNINSMAWYR |
| | (E1D, N93R)-50GS- | RAPGKQRELVASSTNGGSTNYADSVKGRFTISRDNAKR |
| | F0103492E09-FLAG3- | VYLQMNSLTPEDTAVYYCRALLQPSIYDISRTYWGQGT |
| | HIS6 | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASKSILSFAYMRWYRQAPGKQREFVASIAIGGAT |
| | | SYTDSVKGRFTISRDNAKNTVYLQMNSLKPEDTAVYYC |
| | | SAPAGQYRGQGTLVTVSSGAADYKDHDGDYKDHDIDY |
| | | KDDDDKGAAHHHHHH |
| 441 | F010302378:F0103275B05 | DVQLVESGGGLVQPGGSLRLSCAASGSIFNINSMAWYR |
| | (E1D,N93R)-50GS- | RAPGKQRELVASSTNGGSTNYADSVKGRFTISRDNAKR |
| | F0103495F09-FLAG3- | VYLQMNSLTPEDTAVYYCRALLQPSIYDISRTYWGQGT |
| | HIS6 | LVTVSSGGGGSGGGGSGGGGSGGGGSGGGGSGGGGSG |
| | | GGGSGGGGSGGGGSGGGGSEVQLVESGGGLVQAGSSL |
| | | SLSCAASGRALSTYAMGWFRQAPGKEREFVARISRSGIT |
| | | TYYTDSVKGRFTISRDRAKDTVYLQMNSLKPEDTAIYLC |
| | | AADASTNPAGYYLRNRYDYWGQGTLVTVSSGAADYK |
| | | DHDGDYKDHDIDYKDDDDKGAAHHHHHH |
| 442 | F010302379:F0103275B05 | DVQLVESGGGLVQPGGSLRLSCAASGSIFNINSMAWYR |
| | (E1D, N93R)-50GS- | RAPGKQRELVASSTNGGSTNYADSVKGRFTISRDNAKR |
| | F0103500E03(P14A, L108Q)- | VYLQMNSLTPEDTAVYYCRALLQPSIYDISRTYWGQGT |
| | FLAG3-HIS6 | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASGRTFSRYQMGWFRQAPGKEREFVAYISWSGS |
| | | TRYVDSVKGRFTISRDNAKNTVYLQMNSLKPEDTAVYH |
| | | CAAGTAGIISSRPETYDSWGQGTQVTVSSGAADYKDHD |
| | | GDYKDHDIDYKDDDDKGAAHHHHHH |
| 443 | F010302380:F0103275B05 | DVQLVESGGGLVQPGGSLRLSCAASGSIFNINSMA |
| | (E1D, N93R)-50GS- | WYRRAPGKQRELVASSTNGGSTNYADSVKGRFTIS |
| | F0103505D08(L108Q)- | RDNAKRVYLQMNSLTPEDTAVYYCRALLQPSIYDI |
| | FLAG3-HIS6 | SRTYWGQGTLVTVSSGGGGSGGGGSGGGGSGGGG |
| | | SGGGGSGGGGSGGGGSGGGGSGGGGSGGGGSEVQ |
| | | LVESGGGLVQAGGSLRLSCVTSGRTSDLSTMNWFR |
| | | QAPGKEREFVARITRRGSTYYAESVKERFIISRDNA |
| | | KNTVYLQMNSLKPEDTANYYCTAASEMGYHYRG |
| | | QGTQVTVSSGAADYKDHDGDYKDHDIDYKDDDD |
| | | KGAAHHHHHH |
| 444 | F010300191:F0103275B05- | EVQLVESGGGLVQPGGSLRLSCAASGSIFNINSMAWYR |
| | 50GS- | RAPGKQRELVASSTNGGSTNYADSVKGRFTISRDNAKR |
| | F0103240B04-FLAG3- | VYLQMNSLTPEDTAVYYCNALLQPSIYDISRTYWGQGT |
| | HIS6 | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASGGTGRRYAMGWFRQAPGKEREIVAAIRWSA |
| | | MTYYADDGKGRFTISRDNAKNTVYLQMNSLKPEDTAIY |
| | | YCAYTWDYFKYDQVRAYRGWGQGTLVTVSSGAADYK |
| | | DHDGDYKDHDIDYKDDDDKGAAHHHHHH |
| 445 | F010302375 (No Tag): | DVQLVESGGGLVQPGGSLRLSCAASGSIFNINSMAWYR |
| | F0103275B05(E1D, N93R)- | RAPGKQRELVASSTNGGSTNYADSVKGRFTISRDNAKR |
| | 50GS- | VYLQMNSLTPEDTAVYYCRALLQPSIYDISRTYWGQGT |
| | F0103478E09(L108Q) | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASGRAFSTLAMGWFRQAPGKEREFVAAISRNGN |
| | | NSATGDSLKGRFTISRDSTKSTVFLQMNTLKPEDTAVYY |
| | | CAAISTPSASHPYVRKESYRYWGQGTQVTVSS |
| 446 | F010302377 (No | DVQLVESGGGLVQPGGSLRLSCAASGSIFNINSMAWYR |
| | Tag):F0103275B05 | RAPGKQRELVASSTNGGSTNYADSVKGRFTISRDNAKR |
| | (E1D, N93R)-50GS- | VYLQMNSLTPEDTAVYYCRALLQPSIYDISRTYWGQGT |
| | F0103492E09 | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASKSILSFAYMRWYRQAPGKQREFVASIAIGGAT |
| | | SYTDSVKGRFTISRDNAKNTVYLQMNSLKPEDTAVYYC |
| | | SAPAGQYRGQGTLVTVSS |
| 447 | F010302378 (No | DVQLVESGGGLVQPGGSLRLSCAASGSIFNINSMAWYR |
| | Tag):F0103275B05 | RAPGKQRELVASSTNGGSTNYADSVKGRFTISRDNAKR |
| | (E1D, 93R)-50GS- | VYLQMNSLTPEDTAVYYCRALLQPSIYDISRTYWGQGT |
| | F0103495F09 | LVTVSSGGGGSGGGGSGGGGSGGGGSGGGGSGGGGSG |
| | | GGGSGGGGSGGGGSGGGGSEVQLVESGGGLVQAGSSL |
| | | SLSCAASGRALSTYAMGWFRQAPGKEREFVARISRSGIT |
| | | TYYTDSVKGRFTISRDRAKDTVYLQMNSLKPEDTAIYLC |
| | | AADASTNPAGYYLRNRYDYWGQGTLVTVSS |
| 448 | F010302379 (No | DVQLVESGGGLVQPGGSLRLSCAASGSIFNINSMAWYR |
| | Tag):F0103275B05 | RAPGKQRELVASSTNGGSTNYADSVKGRFTISRDNAKR |
| | (E1D, N93R)-50GS- | VYLQMNSLTPEDTAVYYCRALLQPSIYDISRTYWGQGT |
| | F0103500E03(P14A, L108Q) | LVTVSSGGGGSGGGGSGGGGSGGGGSGGGGSGGGGSG |
| | | GGGSGGGGSGGGGSGGGGSEVQLVESGGGLVQAGGSL |
| | | RLSCAASGRTFSRYQMGWFRQAPGKEREFVAYISWSGS |
| | | TRYVDSVKGRFTISRDNAKNTVYLQMNSLKPEDTAVYH |
| | | CAAGTAGIISSRPETYDSWGQGTQVTVSS |
| 449 | F010302380 (No | DVQLVESGGGLVQPGGSLRLSCAASGSIFNINSMA |
| | Tag):F0103275B05 | WYRRAPGKQRELVASSTNGGSTNYADSVKGRFTIS |
| | (E1D, N93R)-50GS- | RDNAKRVYLQMNSLTPEDTAVYYCRALLQPSIYDI |
| | F0103505D08(L108Q) | SRTYWGQGTLVTVSSGGGGSGGGGSGGGGSGGGG |
| | | SGGGGSGGGGSGGGGSGGGGSGGGGSGGGGSEVQ |
| | | LVESGGGLVQAGGSLRLSCVTSGRTSDLSTMNWFR |
| | | QAPGKEREFVARITRRGSTYYAESVKERFIISRDNA |
| | | KNTVYLQMNSLKPEDTANYYCTAASEMGYHYRG |
| | | QGTQVTVSS |
| 450 | F010300191 (No | EVQLVESGGGLVQPGGSLRLSCAASGSIFNINSMA |
| | Tag):F0103275B05- | WYRRAPGKQRELVASSTNGGSTNYADSVKGRFTIS |
| | 50GS-F0103240B04 | RDNAKRVYLQMNSLTPEDTAVYYCNALLQPSIYDI |
| | | SRTYWGQGTLVTVSSGGGGSGGGGSGGGGSGGGG |
| | | SGGGGSGGGGSGGGGSGGGGSGGGGSGGGGSEVQ |
| | | LVESGGGLVQAGGSLRLSCAASGGTGRRYAMGWF |
| | | RQAPGKEREIVAAIRWSAMTYYADDGKGRFTISRD |
| | | NAKNTVYLQMNSLKPEDTAIYYCAYTWDYFKYDQ |
| | | VRAYRGWGQGTLVTVSS |
| 451 | F0103PMP478E09 | EVQLVESGGGLVQAGGSLRLSCAASGRAFSTLAM |
| | | GWFRQAPGKEREFVAAISRNGNNSATGDSLKGRFT |
| | | ISRDSTKSTVFLQMNTLKPEDTAVYYCAAISTPSAS |
| | | HPYVRKESYRYWGQGTQVTVSSAAADYKDHDGD |
| | | YKDHDIDYKDDDDKGAAHHHHHHKAAGGGGG |
| 452 | F0103PMP492E09 | EVQLVESGGGLVQAGGSLRLSCAASKSILSFAYMRWYR |
| | | QAPGKQREFVASIAIGGATSYTDSVKGRFTISRDNAKNT |
| | | VYLQMNSLKPEDTAVYYCSAPAGQYRGQGTLVTVSSA |
| | | AADYKDHDGDYKDHDIDYKDDDDKGAAHHHHHHKA |
| | | AGGGGG |
| 453 | F0103PMP495F09 | EVQLVESGGGLVQAGSSLSLSCAASGRALSTYAMGWFR |
| | | QAPGKEREFVARISRSGITTYYTDSVKGRFTISRDRAKDT |
| | | VY |
| | | LQMNSLKPEDTAIYLCAADASTNPAGYYLRNRYDYWG |
| | | QGTLVTVSSAAADYKDHDGDYKDHDIDYKDDDDKGA |
| | | AHHHHHHKAAGGGGG |
| 454 | F0103PMP500E03 | EVQLVESGGGLVQAGGSLRLSCAASGRTFSRYQMGWF |
| | | RQAPGKEREFVAYISWSGSTRYVDSVKGRFTISRDNAK |
| | | NTVYLQMNSLKPEDTAVYHCAAGTAGIISSRPETYDSW |
| | | GQGTQVTVSSAAADYKDHDGDYKDHDIDYKDDDDKG |
| | | AAHHHHHHKAA |
| 455 | F0103PMP505D08 | EVQLVESGGGLVQAGGSLRLSCVTSGRTSDLSTMNWFR |
| | | QAPGKEREFVARITRRGSTYYAESVKERFIISRDNAKNT |
| | | VYL |
| | | QMNSLKPEDTANYYCTAASEMGYHYRGQGTQVTVSSA |
| | | AADYKDHDGDYKDHDIDYKDDDDKGAAHHHHHHKA |
| | | AGGGGG |
| 456 | Nucleotide sequence | ATGAAAAAGACCGCTATCGCGATTGCAGTGGCACTGG |
| | encoding F0103240B04 | CTGGTTTGGCCACCGTGGCCCAGGCCGAGGTGCAATT |
| | (No tag) | GGTGGAGTCTGGGGGAGGATTGGTGCAGGCTGGGGG |
| | | CTCTCTGAGACTCTCCTGTGCAGCCTCTGGAGGTACA |
| | | GGCAGGAGATATGCCATGGGCTGGTTCCGCCAGGCTC |
| | | CAGGGAAGGAGCGTGAAATTGTAGCAGCGATTAGGT |
| | | GGAGTGCTATGACATACTATGCAGACGACGGGAAGG |
| | | GCCGATTCACCATCTCCAGAGACAACGCCAAGAACAC |
| | | GGTGTATCTCCAAATGAACAGCCTGAAACCTGAGGAC |
| | | ACGGCCATTTATTACTGTGCATACACTTGGGACTATTT |
| | | CAAGTATGACCAAGTCCGAGCGTATCGCGGCTGGGGC |
| | | CAGGGGACCCTGGTCACCGTCTCCTCA |
| 457 | Nucleotide sequence | ATGAAAAAGACCGCTATCGCGATTGCAGTGGCACTGG |
| | encoding F0103478E09 | CTGGTTTGGCCACCGTGGCCCAGGCCGAGGTGCAATT |
| | (No tag) | GGTGGAGTCTGGGGGAGGCTTGGTGCAGGCTGGGGG |
| | | GTCTCTGAGACTCTCCTGTGCTGCCTCTGGACGCGCCT |
| | | TCAGTACCTTGGCCATGGGCTGGTTCCGCCAGGCTCC |
| | | AGGGAAGGAGCGTGAGTTTGTAGCAGCTATTAGCCG |
| | | GAATGGTAATAACTCAGCCACTGGAGACTCCCTGAAG |
| | | GGCCGATTCACCATCTCCAGAGACAGCACCAAGAGC |
| | | ACGGTTTTTCTGCAAATGAATACGCTGAAACCTGAGG |
| | | ACACGGCCGTATATTACTGTGCAGCCATCTCGACACC |
| | | GTCCGCCAGTCATCCATACGTTCGCAAGGAAAGTTAT |
| | | AGATACTGGGGCCAGGGTACCCTGGTCACCGTCTCCT |
| | | CA |
| 458 | Nucleotide sequence | ATGAAAAAGACCGCTATCGCGATTGCAGTGGCACTGG |
| | encoding F0103492E09 | CTGGTTTGGCCACCGTGGCCCAGGCCGAGGTGCAATT |
| | (No tag) | GGTGGAGTCTGGGGGAGGCTTGGTGCAGGCTGGGGG |
| | | ATCTCTGAGACTCTCCTGTGCAGCCTCTAAAAGCATC |
| | | TTAAGTTTCGCTTACATGCGCTGGTACCGCCAGGCTC |
| | | CAGGGAAGCAGCGCGAGTTCGTCGCAAGTATTGCTAT |
| | | TGGAGGTGCCACAAGCTATACAGACTCCGTGAAGGG |
| | | CCGATTCACCATCTCCAGAGACAACGCCAAGAACACG |
| | | GTGTATCTGCAAATGAACAGCCTGAAACCTGAGGACA |
| | | CAGCCGTCTATTACTGTAGTGCACCAGCCGGACAGTA |
| | | TCGGGGCCAGGGGACCCTGGTCACCGTCTCCTCA |
| 459 | Nucleotide sequence | ATGAAAAAGACCGCTATCGCGATTGCAGTGGCACTGG |
| | encoding F0103500E03 | CTGGTTTGGCCACCGTGGCCCAGGCCGAGGTGCAATT |
| | (No tag) | GGTGGAGTCTGGGGGAGGATTGGTGCAGCCGGGGGG |
| | | CTCTCTGAGACTCTCCTGTGCAGCCTCTGGACGCACCT |
| | | TCTCGCGCTATCAGATGGGCTGGTTCCGCCAGGCTCC |
| | | AGGGAAGGAGCGTGAGTTTGTAGCATATATTAGCTGG |
| | | AGTGGTAGTACACGTTATGTTGACTCCGTGAAGGGCC |
| | | GATTCACCATCTCCAGAGACAACGCCAAGAACACGGT |
| | | GTATCTGCAAATGAACAGCCTGAAACCTGAGGACAC |
| | | GGCCGTTTATCACTGTGCAGCAGGGACGGCCGGCATA |
| | | ATATCTAGTAGGCCTGAAACTTATGACTCATGGGGCC |
| | | AGGGGACCCTGGTCACCGTCTCCTCA |
| 460 | Nucleotide sequence | ATGAAAAAGACCGCTATCGCGATTGCAGTGGCACTGG |
| | encoding F0103505D08 | CTGGTTTGGCCACCGTGGCCCAGGCCGAGGTGCAATT |
| | (No tag) | GGTGGAGTCTGGGGGAGGATTGGTGCAGGCTGGGGG |
| | | CTCTCTGAGACTCTCCTGTGTAACCTCTGGACGCACCT |
| | | CCGATTTGTCTACCATGAACTGGTTCCGCCAGGCTCC |
| | | AGGAAAGGAGCGTGAGTTTGTCGCACGCATCACTCGG |
| | | CGTGGTAGCACATACTATGCAGAGTCCGTGAAGGAAC |
| | | GATTCATCATCTCCAGAGACAACGCCAAGAACACGGT |
| | | GTATTTGCAAATGAACAGCCTGAAACCAGAGGACAC |
| | | GGCCAATTATTACTGTACTGCAGCCTCAGAAATGGGA |
| | | TATCACTACAGGGGCCAGGGGACCCTGGTCACCGTCT |
| | | CCTCA |
| 461 | Nucleotide sequence | ATGAAAAAGACCGCTATCGCGATTGCAGTGGCACTGG |
| | encoding F0103495F09 | CTGGTTTGGCCACCGTGGCCCAGGCCGAGGTGCAATT |
| | (No tag) | GGTGGAGTCTGGGGGAGGTTTGGTGCAGGCTGGAAG |
| | | CTCTCTGAGTCTCTCCTGTGCAGCCTCTGGACGCGCCT |
| | | TGAGTACATACGCCATGGGCTGGTTCCGCCAGGCTCC |
| | | AGGGAAGGAGCGTGAGTTTGTAGCACGTATTAGCCG |
| | | GAGCGGGATTACAACATACTATACAGACTCCGTGAAG |
| | | GGCCGATTCACCATCTCCAGAGACCGCGCCAAGGACA |
| | | CGGTGTATCTGCAAATGAACAGCCTGAAACCTGAGGA |
| | | CACGGCCATTTATTTGTGTGCAGCAGACGCCTCAACC |
| | | AATCCTGCTGGATACTACCTTCGGAATCGTTATGACT |
| | | ACTGGGGCCAGGGGACCCTGGTCACCGTCTCCTCA |
| 462 | 50GS linker | GGGGSGGGGSGGGGSGGGGSGGGGSGGGGSGGG |
| | | GSGGGGSGGGGSGGGGS |
| 463 | Nucleotide sequence | ATGAGATTTCCTTCAATTTTTACTGCTGTTTTATT |
| | encoding F010302375 | CGCAGCATCCTCCGCATTAGCTGCTCCAGTCAAC |
| | (No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGATTTATGACATTAGTCG |
| | | CACATATTGGGGCCAGGGGACCCTGGTCACGGTC |
| | | TCCTCCGGAGGTGGTGGCAGCGGTGGAGGTGGTT |
| | | CTGGGGGTGGCGGTAGTGGCGGTGGTGGCTCAG |
| | | GTGGCGGTGGGTCAGGCGGTGGTGGCAGTGGTG |
| | | GGGGTGGCAGCGGTGGCGGTGGATCTGGTGGAG |
| | | GTGGTTCTGGAGGTGGAGGATCCGAGGTGCAGTT |
| | | GGTGGAGTCTGGGGGAGGCTTGGTGCAGGCTGG |
| | | GGGGTCTCTGAGACTCTCCTGTGCTGCCTCTGGA |
| | | CGCGCCTTCAGTACCTTGGCCATGGGCTGGTTCC |
| | | GCCAGGCTCCAGGGAAGGAGCGTGAGTTTGTAG |
| | | CAGCTATTAGCCGGAATGGTAATAACTCAGCCAC |
| | | TGGAGACTCCCTGAAGGGCCGATTCACCATCTCC |
| | | AGAGACAGCACCAAGAGCACGGTTTTTCTGCAA |
| | | ATGAATACGCTGAAACCTGAGGACACGGCCGTA |
| | | TATTACTGTGCAGCCATCTCGACACCGTCCGCCA |
| | | GTCATCCATACGTTCGCAAGGAAAGTTATAGATA |
| | | CTGGGGCCAGGGTACCCAGGTCACCGTCTCCTCA |
| 464 | Nucleotide sequence | ATGAGATTTCCTTCAATTTTTACTGCTGTTTTATT |
| | encoding F010302377 | CGCAGCATCCTCCGCATTAGCTGCTCCAGTCAAC |
| | (No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGAT |
| | | TTATGACATTAGTCGCACATATTGGGGCCAGGGG |
| | | ACCCTGGTCACGGTCTCCTCCGGAGGTGGTGGCA |
| | | GCGGTGGAGGTGGTTCTGGGGGTGGCGGTAGTG |
| | | GCGGTGGTGGCTCAGGTGGCGGTGGGTCAGGCG |
| | | GTGGTGGCAGTGGTGGGGGTGGCAGCGGTGGCG |
| | | GTGGATCTGGTGGAGGTGGTTCTGGAGGTGGAG |
| | | GATCCGAGGTGCAGTTGGTGGAGTCTGGGGGAG |
| | | GCTTGGTGCAGGCTGGGGGATCTCTGAGACTCTC |
| | | CTGTGCAGCCTCTAAAAGCATCTTAAGTTTCGCT |
| | | TACATGCGCTGGTACCGCCAGGCTCCAGGGAAG |
| | | CAGCGCGAGTTCGTCGCAAGTATTGCTATTGGAG |
| | | GTGCCACAAGCTATACAGACTCCGTGAAGGGCC |
| | | GATTCACCATCTCCAGAGACAACGCCAAGAACA |
| | | CGGTGTATCTGCAAATGAACAGCCTGAAACCTGA |
| | | GGACACAGCCGTCTATTACTGTAGTGCACCAGCC |
| | | GGACAGTATCGGGGCCAGGGGACCCTGGTCACC |
| | | GTCTCCTCA |
| 465 | Nucleotide sequence | ATGAGATTTCCTTCAATTTTTACTGCTGTTTTATT |
| | encoding F010302378 | CGCAGCATCCTCCGCATTAGCTGCTCCAGTCAAC |
| | (No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGAT |
| | | TTATGACATTAGTCGCACATATTGGGGCCAGGGG |
| | | ACCCTGGTCACGGTCTCCTCCGGAGGTGGTGGCA |
| | | GCGGTGGAGGTGGTTCTGGGGGTGGCGGTAGTG |
| | | GCGGTGGTGGCTCAGGTGGCGGTGGGTCAGGCG |
| | | GTGGTGGCAGTGGTGGGGGTGGCAGCGGTGGCG |
| | | GTGGATCTGGTGGAGGTGGTTCTGGAGGTGGAG |
| | | GATCCGAGGTGCAGTTGGTGGAGTCTGGGGGAG |
| | | GTTTGGTGCAGGCTGGAAGCTCTCTGAGTCTCTC |
| | | CTGTGCAGCCTCTGGACGCGCCTTGAGTACATAC |
| | | GCCATGGGCTGGTTCCGCCAGGCTCCAGGGAAG |
| | | GAGCGTGAGTTTGTAGCACGTATTAGCCGGAGCG |
| | | GGATTACAACATACTATACAGACTCCGTGAAGG |
| | | GCCGATTCACCATCTCCAGAGACCGCGCCAAGG |
| | | ACACGGTGTATCTGCAAATGAACAGCCTGAAAC |
| | | CTGAGGACACGGCCATTTATTTGTGTGCAGCAGA |
| | | CGCCTCAACCAATCCTGCTGGATACTACCTTCGG |
| | | AATCGTTATGACTACTGGGGCCAGG |
| | | GGACCCTGGTCACCGTCTCCTCA |
| 466 | Nucleotide sequence | ATGAGATTTCCTTCAATTTTTACTGCTGTTTTATT |
| | encoding F010302379 | CGCAGCATCCTCCGCATTAGCTGCTCCAGTCAAC |
| | (No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGAT |
| | | TTATGACATTAGTCGCACATATTGGGGCCAGGGG |
| | | ACCCTGGTCACGGTCTCCTCCGGAGGTGGTGGCA |
| | | GCGGTGGAGGTGGTTCTGGGGGTGGCGGTAGTG |
| | | GCGGTGGTGGCTCAGGTGGCGGTGGGTCAGGCG |
| | | GTGGTGGCAGTGGTGGGGGTGGCAGCGGTGGCG |
| | | GTGGATCTGGTGGAGGTGGTTCTGGAGGTGGAG |
| | | GATCCGAGGTGCAGTTGGTGGAGTCTGGGGGAG |
| | | GATTGGTGCAGGCTGGGGGCTCTCTGAGACTCTC |
| | | CTGTGCAGCCTCTGGACGCACCTTCTCGAGGTAT |
| | | CAGATGGGCTGGTTCCGCCAGGCTCCAGGGAAG |
| | | GAGCGTGAGTTTGTAGCATATATTAGCTGGAGTG |
| | | GTAGTACACGTTATGTTGACTCCGTGAAGGGCCG |
| | | ATTCACCATCTCCAGAGACAACGCCAAGAACAC |
| | | GGTGTATCTGCAAATGAACAGCCTGAAACCTGA |
| | | GGACACGGCCGTTTATCACTGTGCAGCAGGGAC |
| | | GGCCGGCATAATATCTAGTAGGCCTGAAACTTAT |
| | | GACTCATGGGGCCAGGGGACCCAGG |
| | | TCACCGTCTCCTCA |
| 467 | Nucleotide sequence | ATGAGATTTCCTTCAATTTTTACTGCTGTTTTATT |
| | encoding | CGCAGCATCCTCCGCATTAGCTGCTCCAGTCAAC |
| | F010302380(No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGAT |
| | | TTATGACATTAGTCGCACATATTGGGGCCAGGGG |
| | | ACCCTGGTCACGGTCTCCTCCGGAGGTGGTGGCA |
| | | GCGGTGGAGGTGGTTCTGGGGGTGGCGGTAGTG |
| | | GCGGTGGTGGCTCAGGTGGCGGTGGGTCAGGCG |
| | | GTGGTGGCAGTGGTGGGGGTGGCAGCGGTGGCG |
| | | GTGGATCTGGTGGAGGTGGTTCTGGAGGTGGAG |
| | | GATCCGAGGTGCAGTTGGTGGAGTCTGGGGGAG |
| | | GATTGGTGCAGGCTGGGGGCTCTCTGAGACTCTC |
| | | CTGTGTAACCTCTGGACGCACCTCCGATTTGTCT |
| | | ACCATGAACTGGTTCCGCCAGGCTCCAGGAAAG |
| | | GAGCGTGAGTTTGTCGCACGCATCACTCGGCGTG |
| | | GTAGCACATACTATGCAGAGTCCGTGAAGGAAC |
| | | GATTCATCATCTCCAGAGACAACGCCAAGAACA |
| | | CGGTGTATTTGCAAATGAACAGCCTGAAACCAG |
| | | AGGACACGGCCAATTATTACTGTACTGCAGCCTC |
| | | AGAAATGGGATATCACTACAGGGGCCAGGGGAC |
| | | CCAGGTCACCGTCTCCTCA |
| 468 | Nucleotide sequence | ATGAGATTTCCTTCAATTTTTACTGCTGTTTTATT |
| | encoding F010302391 | CGCAGCATCCTCCGCATTAGCTGCTCCAGTCAAC |
| | (No tag) | ACTACAACAGAAGATGAAACGGCACAAATTCCG |
| | | GCTGAAGCTGTCATCGGTTACTCAGATTTAGAAG |
| | | GGGATTTCGATGTTGCTGTTTTGCCATTTTCCAAC |
| | | AGCACAAATAACGGGTTATTGTTTATAAATACTA |
| | | CTATTGCCAGCATTGCTGCTAAAGAAGAAGGGGT |
| | | ATCTCTCGAAAAGAGAGACGTGCAATTGGTGGA |
| | | GTCTGGGGGAGGCTTGGTGCAGCCTGGGGGGTCT |
| | | CTGAGACTCTCCTGTGCAGCCTCTGGAAGCATCT |
| | | TCAATATCAACAGTATGGCCTGGTATCGCCGGGC |
| | | TCCAGGGAAGCAGCGCGAATTGGTCGCAAGTAG |
| | | CACCAATGGTGGTAGTACAAACTATGCAGACTCC |
| | | GTGAAGGGCCGATTCACCATCTCTAGAGACAAC |
| | | GCCAAACGGGTGTATCTGCAAATGAACAGCCTG |
| | | ACACCTGAGGACACGGCCGTCTATTATTGTCGTG |
| | | CACTGCTACAACCGTCGAT |
| | | TTATGACATTAGTCGCACATATTGGGGCCAGGGG |
| | | ACCCTGGTCACGGTCTCCTCCGGAGGTGGTGGCA |
| | | GCGGTGGAGGTGGTTCTGGGGGTGGCGGTAGTG |
| | | GCGGTGGTGGCTCAGGTGGCGGTGGGTCAGGCG |
| | | GTGGTGGCAGTGGTGGGGGTGGCAGCGGTGGCG |
| | | GTGGATCTGGTGGAGGTGGTTCTGGAGGTGGAG |
| | | GATCCGAGGTGCAGTTGGTGGAGTCTGGGGGAG |
| | | GATTGGTGCAGGCTGGGGGCTCTCTGAGACTCTC |
| | | CTGTGCAGCCTCTGGACGCACCGCTAGTATCTAT |
| | | GCCATGGCCTGGTTCCGCCAGGCTCAGGGGAAG |
| | | GAGCGTGAATTTGTCGCAGTTATTACCCGGAGTG |
| | | GTGGAACGATCGTCTATGCAGACTCCGTGAAGG |
| | | GCCGATTCACCATCTCCAGAGACGACGCCAAGA |
| | | ACACTGTGTGGTTGCAAATGAGCGCTCTGAGACC |
| | | TGAGGACACAGCCGTATATTTCTGTAATGCGGTT |
| | | GCGGTCGAAGACGGGATGAACGTTATGAATTATT |
| | | GGGGCCAGGGGACCCTGGTCACCG |
| | | TCTCCTCA |
| 469 | Human IgG1 HC | ASTKGPSVFPLAPSSKSTSGGTAALGCLVKDYFPEP |
| | constant domain | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 470 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (L234A L235A D265S) | PSSSLGTQTYICNVNHKPSNTKVDKKVEPKSCDKT |
| | | HTCPPCPAPEAAGGPSVFLFPPKPKDTLMISRTPEVT |
| | | CVVVSVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 471 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (L234A L235A P329G) | PSSSLGTQTYICNVNHKPSNTKVDKKVEPKSCDKT |
| | | HTCPPCPAPEAAGGPSVFLFPPKPKDTLMISRTPEVT |
| | | CVVVSVSHEDPEVKFNWYVDGVEVHNAKTKPREE |
| | | QYNSTYRVVSVLTVLHQDWLNGKEYKCKVSNKA |
| | | LGAPIEKTISKAKGQPREPQVYTLPPSRDELTKNQV |
| | | SLTCLVKGFYPSDIAVEWESNGQPENNYKTTPPVL |
| | | DSDGSFFLYSKLTVDKSRWQQGNVFSCSVMHEAL |
| | | HNHYTQKSLSLSPGK |
| 472 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (L235E) | PSSSLGTQTYICNVNHKPSNTKVDKKVEPKSCDKT |
| | | HTCPPCPAPELEGGPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 473 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (D265A) | PSSSLGTQTYICNVNHKPSNTKVDKKVEPKSCDKT |
| | | HTCPPCPAPELLGGPSVFLFPPKPKDTLMISRTPEVT |
| | | CVVVAVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 474 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (D265A N297G) | PSSSLGTQTYICNVNHKPSNTKVDKKVEPKSCDKT |
| | | HTCPPCPAPELLGGPSVFLFPPKPKDTLMISRTPEVT |
| | | CVVVAVSHEDPEVKFNWYVDGVEVHNAKTKPREE |
| | | QYGSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 475 | Human IgG1 HC | ASTKGPSVFPLAPSSKSTSGGTAALGCLVKDYFPEP |
| | Constant domain | VTVSWNSGALTSGVHTFPAVLQSSGLYSLSSVVTV |
| | (E233A/L235A) | PSSSLGTQTYICNVNHKPSNTKVDKKVEPKSCDKT |
| | | HTCPPCPAPALAGGPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPAPIEKTISKAKGQPREPQVYTLPPSRDELTKNQVS |
| | | LTCLVKGFYPSDIAVEWESNGQPENNYKTTPPVLD |
| | | SDGSFFLYSKLTVDKSRWQQGNVFSCSVMHEALH |
| | | NHYTQKSLSLSPGK |
| 476 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | | PSSNFGTQTYTCNVDHKPSNTKVDKTVERKCCVEC |
| | | PPCPAPPVAGPSVFLFPPKPKDTLMISRTPEVTCVVV |
| | | DVSHEDPEVQFNWYVDGVEVHNAKTKPREEQFNS |
| | | TFRVVSVLTVVHQDWLNGKEYKCKVSNKGLPAPI |
| | | EKTISKTKGQPREPQVYTLPPSREEMTKNQVSLTCL |
| | | VKGFYPSDISVEWESNGQPENNYKTTPPMLDSDGS |
| | | FFLYSKLTVDKSRWQQGNVFSCSVMHEALHNHYT |
| | | QKSLSLSPGK |
| 477 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | (D265S) | PSSNFGTQTYTCNVDHKPSNTKVDKTVERKCCVEC |
| | | PPCPAPPVAGPSVFLFPPKPKDTLMISRTPEVTCVVV |
| | | SVSHEDPEVQFNWYVDGVEVHNAKTKPREEQFNS |
| | | TFRVVSVLTVVHQDWLNGKEYKCKVSNKGLPAPI |
| | | EKTISKTKGQPREPQVYTLPPSREEMTKNQVSLTCL |
| | | VKGFYPSDISVEWESNGQPENNYKTTPPMLDSDGS |
| | | FFLYSKLTVDKSRWQQGNVFSCSVMHEALHNHYT |
| | | QKSLSLSPGK |
| 478 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | (P329G) | PSSNFGTQTYTCNVDHKPSNTKVDKTVERKCCVEC |
| | | PPCPAPPVAGPSVFLFPPKPKDTLMISRTPEVTCVVV |
| | | DVSHEDPEVQFNWYVDGVEVHNAKTKPREEQFNS |
| | | TFRVVSVLTVVHQDWLNGKEYKCKVSNKGLGAPI |
| | | EKTISKTKGQPREPQVYTLPPSREEMTKNQVSLTCL |
| | | VKGFYPSDISVEWESNGQPENNYKTTPPMLDSDGS |
| | | FFLYSKLTVDKSRWQQGNVFSCSVMHEALHNHYT |
| | | QKSLSLSPGK |
| 479 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | (D265A) | PSSNFGTQTYTCNVDHKPSNTKVDKTVERKCCVEC |
| | | PPCPAPPVAGPSVFLFPPKPKDTLMISRTPEVTCVVV |
| | | AVSHEDPEVQFNWYVDGVEVHNAKTKPREEQFNS |
| | | TFRVVSVLTVVHQDWLNGKEYKCKVSNKGLPAPI |
| | | EKTISKTKGQPREPQVYTLPPSREEMTKNQVSLTCL |
| | | VKGFYPSDISVEWESNGQPENNYKTTPPMLDSDGS |
| | | FFLYSKLTVDKSRWQQGNVFSCSVMHEALHNHYT |
| | | QKSLSLSPGK |
| 480 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | (D265A N297G) | PSSNFGTQTYTCNVDHKPSNTKVDKTVERKCCVEC |
| | | PPCPAPPVAGPSVFLFPPKPKDTLMISRTPEVTCVVV |
| | | AVSHEDPEVQFNWYVDGVEVHNAKTKPREEQFGS |
| | | TFRVVSVLTVVHQDWLNGKEYKCKVSNKGLPAPI |
| | | EKTISKTKGQPREPQVYTLPPSREEMTKNQVSLTCL |
| | | VKGFYPSDISVEWESNGQPENNYKTTPPMLDSDGS |
| | | FFLYSKLTVDKSRWQQGNVFSCSVMHEALHNHYT |
| | | QKSLSLSPGK |
| 481 | Human IgG2 HC | ASTKGPSVFPLAPCSRSTSESTAALGCLVKDYFPEP |
| | Constant domain | VTVSWNSGALTSGVHTFPAVLQSSGLYSLSSVVTV |
| | (V234A G237A P238S | PSSNFGTQTYTCNVDHKPSNTKVDKTVERKCCVEC |
| | H268A V309L A330S | PPCPAPPAAASSVFLFPPKPKDTLMISRTPEVTCVVV |
| | P331S X378S/A)(See | DVSAEDPEVQFNWYVDGVEVHNAKTKPREEQFNS |
| | IgGsigma SEQ ID | TFRVVSVLTVLHQDWLNGKEYKCKVSNKGLPSSIE |
| | No: 78 in | KTISKTKGQPREPQVYTLPPSREEMTKNQVSLTCLV |
| | WO2017079112) | KGFYPSDIXVEWESNGQPENNYKTTPPMLDSDGSF |
| | | FLYSKLTVDKSRWQQGNVFSCSVMHEALHNHYTQ |
| | | KSLSLSPGK |
| 482 | Human IgG4 HC | ASTKGPSVFPLAPCSRSTSESTAALGCLVKDYFPEP |
| | Constant domain | VTVSWNSGALTSGVHTFPAVLQSSGLYSLSSVVTV |
| | (S228P) | PSSSLGTKTYTCNVDHKPSNTKVDKRVESKYGPPC |
| | | PPCPAPEFLGGPSVFLFPPKPKDTLMISRTPEVTCVV |
| | | VDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLTCL |
| | | VKGFYPSDIAVEWESNGQPENNYKTTPPVLDSDGS |
| | | FFLYSRLTVDKSRWQEGNVFSCSVMHEALHNHYT |
| | | QKSLSLSLGK |
| 483 | Human IgG4 HC | ASTKGPSVFPLAPCSRSTSESTAALGCLVKDYFPEP |
| | Constant domain | VTVSWNSGALTSGVHTFPAVLQSSGLYSLSSVVTV |
| | (S228P P329G) | PSSSLGTKTYTCNVDHKPSNTKVDKRVESKYGPPC |
| | | PPCPAPEFLGGPSVFLFPPKPKDTLMISRTPEVTCVV |
| | | VDVSQEDPEVQFNWYVDGVEVHNAKTKPREEQFN |
| | | STYRVVSVLTVLHQDWLNGKEYKCKVSNKGLGSS |
| | | IEKTISKAKGQPREPQVYTLPPSQEEMTKNQVSLTC |
| | | LVKGFYPSDIAVEWESNGQPENNYKTTPPVLDSDG |
| | | SFFLYSRLTVDKSRWQEGNVFSCSVMHEALHNHY |
| | | TQKSLSLSLGK |
| 484 | Human IgG4 HC | ASTKGPSVFPLAPCSRSTSESTAALGCLVKDYFPEP |
| | Constant domain | VTVSWNSGALTSGVHTFPAVLQSSGLYSLSSVVTV |
| | (S228P D265A) | PSSSLGTKTYTCNVDHKPSNTKVDKRVESKYGPPC |
| | | PPCPAPEFLGGPSVFLFPPKPKDTLMISRTPEVTCVV |
| | | VAVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLTCL |
| | | VKGFYPSDIAVEWESNGQPENNYKTTPPVLDSDGS |
| | | FFLYSRLTVDKSRWQEGNVFSCSVMHEALHNHYT |
| | | QKSLSLSLGK |
| 485 | Human IgG4 HC | ASTKGPSVFPLAPCSRSTSESTAALGCLVKDYFPEP |
| | Constant domain | VTVSWNSGALTSGVHTFPAVLQSSGLYSLSSVVTV |
| | (S228P D265A N297G) | PSSSLGTKTYTCNVDHKPSNTKVDKRVESKYGPPC |
| | | PPCPAPEFLGGPSVFLFPPKPKDTLMISRTPEVTCVV |
| | | VAVSQEDPEVQFNWYVDGVEVHNAKTKPREEQFG |
| | | STYRVVSVLTVLHQDWLNGKEYKCKVSNKGLPSSI |
| | | EKTISKAKGQPREPQVYTLPPSQEEMTKNQVSLTCL |
| | | VKGFYPSDIAVEWESNGQPENNYKTTPPVLDSDGS |
| | | FFLYSRLTVDKSRWQEGNVFSCSVMHEALHNHYT |
| | | QKSLSLSLGK |
| 486 | F010301657 | MKKTAIAIAVALAGLATVAQAEVQLVESGGGLVQPGGS |
| | | LRLSCAASVRPFSTSAMGWFRQAPEKEREAVAGILWNG |
| | | IVTYYADSVKGRFTISRDNAKNEVYLQMNKLKPEDTAV |
| | | YYCALDRAYQGRSFSAKEYEYWGQGTLVTVSS |
| 487 | F010301658 | MKKTAIAIAVALAGLATVAQAEVQLVESGGGLVQPGGS |
| | | LRLSCAASVRPFSTSAMGWFRQAPEKEREAVAAILWNG |
| | | IVTYYADSVKGRFTISRDNAKNEVYLQMNKLKPEDTAV |
| | | YYCALDRAYQGRSFSAKEYEYWGQGTLVTVSS |
| 488 | F010301659 | MKKTAIAIAVALAGLATVAQAEVQLVESGGGLVQPGGS |
| | | LRLSCAASVRPFSTSAMGWFRQAPEKEREAVAAILWNG |
| | | IVTYYADSVKGRFTISRDNAKNEVYLQMNKLKPEDTAV |
| | | YYCALDRAYGGRSFSAYEYEYWGQGTLVTVSS |
| 489 | F010301661 | MKKTAIAIAVALAGLATVAQAEVQLVESGGGLVQPGGS |
| | | LRLSCAASVRPFSTSAMGWFRQAPEKEREAVAAILWNG |
| | | IVTYYADSVKGRFTISRDNAKNEVYLQMNKLKPEDTAV |
| | | YYCALDRDYGGRSFSAKEYEYWGQGTLVTVSS |
| 490 | F010301662 | MKKTAIAIAVALAGLATVAQAEVQLVESGGGLVQPGGS |
| | | LRLSCAASVRPFSTSAMGWFRQAPEKEREAVAGILWNG |
| | | IVTYYADSVKGRFTISRDNAKNEVYLQMNKLKPEDTAV |
| | | YYCALDRAYQGRSFSAYEYEYWGQGTLVTVSSA |
| 491 | F010301663 | MKKTAIAIAVALAGLATVAQAEVQLVESGGGLVQPGGS |
| | | LRLSCAASVRPFSTSAMGWFRQAPEKEREAVAGILWNG |
| | | IVTYYADSVKGRFTISRDNAKNEVYLQMNKLKPEDTAV |
| | | YYCALDRAYGGRSFSAKEYEYWGQGTLVTVSS |
| 492 | F010301664 | MKKTAIAIAVALAGLATVAQAEVQLVESGGGLVQPGGS |
| | | LRLSCAASVRPFSTSAMGWFRQAPEKEREAVAGILWNG |
| | | IVTYYADSVKGRFTISRDNAKNEVYLQMNKLKPEDTAV |
| | | YYCALDRDYQGRSFSAKEYEYWGQGTLVTVSS |
| 493 | F010301665 | MKKTAIAIAVALAGLATVAQAEVQLVESGGGLVQPGGS |
| | | LRLSCAASVRPFSTSAMGWFRQAPEKEREAVAAILWNG |
| | | IVTYYADSVKGRFTISRDNAKNEVYLQMNKLKPEDTAV |
| | | YYCALDRAYGGRSFSAKEYEYWGQGTLVTV |
| 494 | F010301666 | MKKTAIAIAVALAGLATVAQAEVQLVESGGGLVQPGGS |
| | | LRLSCAASVRPFSTSAMGWFRQAPEKEREAVAAILWNG |
| | | IVTYYADSVKGRFTISRDNAKNEVYLQMNKLKPEDTAV |
| | | YYCALDRDYQGRSFSAKEYEYWGQGTLVTVSS |
| 495 | F010301867 | EVQLVESGGGLVQPGGSLRLSCATTSRQFIRDVFTG |
| | | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSS |
| 496 | F010301878 | EVQLVESGGGLVQPGGSLRLSCATTSRAFIRDVFTG |
| | | WYRRVPGKERELVARIYNEGNTNYADFAKGRFSIS |
| | | RDNAKKMVTLRMSNLKPEDTGVYYCLFSGTINTG |
| | | REYRSGDYWGQGTLVTVSS |
| 497 | F010301879 | EVQLVESGGGLVQPGGSLRLSCATTSRAFIRDVFTG |
| | | WYRRVPGKERELVARIYNQGNTNYADFAKGRFSIS |
| | | RDNAKKMVTLRMSNLKPEDTGVYYCLFSGTINTG |
| | | REYRSGDYWGQGTLVTVSS |
| 498 | F010301880 | EVQLVESGGGLVQPGGSLRLSCATTSRAFIRDVFTG |
| | | WYRRVPGKERELVARIYNSGNTNYADFAKGRFSIS |
| | | RDNAKKMVTLRMSNLKPEDTGVYYCLFSGTINTG |
| | | REYRSGDYWGQGTLVTVSS |
| 499 | F010301881 | EVQLVESGGGLVQPGGSLRLSCATTSRAFIRDVFTG |
| | | WYRRVPGKERELVARIYNWGNTNYADFAKGRFSI |
| | | SRDNAKKMVTLRMSNLKPEDTGVYYCLFSGTINTG |
| | | REYRSGDYWGQGTLVTVSS |
| 500 | F010301882 | EVQLVESGGGLVQPGGSLRLSCATTSRAFVRDVFT |
| | | GWYRRVPGKERELVARIYNGGNTNYADFAKGRFSI |
| | | SRDNAKKMVTLRMSNLKPEDTGVYYCLFSGTINTG |
| | | REYRSGDYWGQGTLVTVSS |
| 501 | F010301883 | EVQLVESGGGLVQPGGSLRLSCATTSRAFIRDVFTG |
| | | WYRRVPGKERELVARVYNGGNTNYADFAKGRFSI |
| | | SRDNAKKMVTLRMSNLKPEDTGVYYCLFSGTINTG |
| | | REYRSGDYWGQGTLVTVSS |
| 502 | F010301884 | EVQLVESGGGLVQPGGSLRLSCATTSRAFIRDVFTG |
| | | WYRRVPGKERELVARIYAGGNTNYADFAKGRFSIS |
| | | RDNAKKMVTLRMSNLKPEDTGVYYCLFSGTINTG |
| | | REYRSGDYWGQGTLVTVSS |
| 503 | F010301885 | EVQLVESGGGLVQPGGSLRLSCATTSRAFIRDVFTG |
| | | WYRRVPGKERELVARIYEGGNTNYADFAKGRFSIS |
| | | RDNAKKMVTLRMSNLKPEDTGVYYCLFSGTINTG |
| | | REYRSGDYWGQGTLVTVSS |
| 504 | F010301886 | EVQLVESGGGLVQPGGSLRLSCATTSRAFIRDVFTG |
| | | WYRRVPGKERELVARIYNGGNTQYADFAKGRFSIS |
| | | RDNAKKMVTLRMSNLKPEDTGVYYCLFSGTINTG |
| | | REYRSGDYWGQGTLVTVSS |
| 505 | F010301887 | EVQLVESGGGLVQPGGSLRLSCATTSRAFIQDVFTG |
| | | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSS |
| 506 | F010301888 | EVQLVESGGGLVQPGGSLRLSCATTHRAFIRDVFT |
| | | GWYRRVPGKERELVARIYNGGNTNYADFAKGRFSI |
| | | SRDNAKKMVTLRMSNLKPEDTGVYYCLFSGTINTG |
| | | REYRSGDYWGQGTLVTVSS |
| 507 | F010301889 | EVQLVESGGGLVQPGGSLRLSCATTSRAFIRDVFTG |
| | | WYRRVPGKERELVARIYNGGNTNYADFAKGRFSIS |
| | | RDNAKKMVTLRMSNLKPEDTGVYYCLFAGTINTG |
| | | REYRSGDYWGQGTLVTVSS |
| 508 | F010301890 | EVQLVESGGGLVQPGGSLRLSCATTSRAFIRDVFVG |
| | | WYRRVPGKERELVARIYNGGNTNYADFAKGRFSIS |
| | | RDNAKKMVTLRMSNLKPEDTGVYYCLFSGTINTG |
| | | REYRSGDYWGQGTLVTVSS |
| 509 | F010301891 | EVQLVESGGGLVQPGGSLRLSCATTSRAFIRDVFTG |
| | | WYRRVPGKERELVARIYNGGNVNYADFAKGRFSIS |
| | | RDNAKKMVTLRMSNLKPEDTGVYYCLFSGTINTG |
| | | REYRSGDYWGQGTLVTVSS |
| 510 | F010300534 | EVQLVESGGGLVQPGGSLRLSCAASGMLFNANTQ |
| | | GWYRQAPGKQRELVAFIFSGGYVNYVDSVKGRFTI |
| | | SRDNAKRTMYLQMNSLKPEDSAIYYCSLSRYLGQG |
| | | TLVTVSS |
| 511 | F010300535 | EVQLVESGGGLVQPGGSLRLSCAASGMLFNANTQ |
| | | GWYRQAPGKQRELVAFIFWGGYTNYVDSVKGRFT |
| | | ISRDNAKRTMYLQMNSLKPEDSAIYYCSLSRYLGQ |
| | | GTLVTVSS |
| 512 | F010300536 | EVQLVESGGGLVQPGGSLRLSCAASGMLFNANTQ |
| | | GWYRQAPGKQRELVAFIFSGGYTTYVDSVKGRFTI |
| | | SRDNAKRTMYLQMNSLKPEDSAIYYCSLSRYLGQG |
| | | TLVTVSS |
| 513 | F010301055 | EVQLVESGGGLVQPGGSLRLSCAASGMLFNANTQ |
| | | GWYRQAPGKQRELVAFIFWGGYVNYVDSVKGRFT |
| | | ISRDNAKRTMYLQMNSLKPEDSAIYYCSLSRYLGQ |
| | | GTLVTVSS |
| 514 | F010301059 | EVQLVESGGGLVQPGGSLRLSCAASGMLFNANTQ |
| | | GWYRQAPGKQRELVAFIFSGGYVNYNDSVKGRFTI |
| | | SRDNAKRTMYLQMNSLKPEDSAIYYCALSRYQGQ |
| | | GTLVTVSS |
| 515 | F010301080 | EVQLVESGGGLVQPGGSLRLSCAASGMLFNANTQ |
| | | GWYRQAPGKQRELVAFIFWGGYVTYNDSVKGRFT |
| | | ISRDNAKRTMYLQMNSLKPEDSAIYYCSLSRYLGQ |
| | | GTLVTVSS |
| 516 | F010301090 | EVQLVESGGGLVQPGGSLRLSCAASGMLFNANTQ |
| | | GWYRQAPGKQRELVAFIFSGGWTTYNDSVKGRFTI |
| | | SRDNAKRTMYLQMNSLKPEDSAIYYCSLSRYQGQ |
| | | GTLVTVSS |
| 517 | F010301099 | EVQLVESGGGLVQPGGSLRLSCAASGMLFNANTQ |
| | | GWYRQAPGKQRELVAFIFSGGWVTYVDSVKGRFTI |
| | | SRDNAKRTMYLQMNSLKPEDSAIYYCSLSRYLGQG |
| | | TLVTVSS |
| 518 | F010301111 | EVQLVESGGGLVQPGGSLRLSCAASGMLFNANTQ |
| | | GWYRQAPGKQRELVAFIFSGGYVNYNDSVKGRFTI |
| | | SRDNAKRTMYLQMNSLKPEDSAIYYCALSRYLGQ |
| | | GTLVTVSS |
| 519 | F010301113 | EVQLVESGGGLVQPGGSLRLSCAASGMLFNRNTQ |
| | | GWYRQAPGKQRELVAFIFSGGYTNYNDSVKGRFTI |
| | | SRDNAKRTMYLQMNSLKPEDSAIYYCSLSVYQGQ |
| | | GTLVTVSS |
| 520 | F010301126 | EVQLVESGGGLVQPGGSLRLSCAASGMLFNRNTQ |
| | | GWYRQAPGKQRELVAFIFSGGYVTYNDSVKGRFTI |
| | | SRDNAKRTMYLQMNSLKPEDSAIYYCSLSRYLGQG |
| | | TLVTVSS |
| 521 | F010301129 | EVQLVESGGGLVQPGGSLRLSCAASGMLFNANTQ |
| | | GWYRQAPGKQRELVAFIFSGGWTNYNDSVKGRFTI |
| | | SRDNAKRTMYLQMNSLKPEDSAIYYCALSRYQGQ |
| | | GTLVTVSS |
| 522 | F010301138 | EVQLVESGGGLVQPGGSLRLSCAASGMLFYANTQ |
| | | GWYRQAPGKQRELVAFIFSGGYTNYNDSVKGRFTI |
| | | SRDNAKRTMYLQMNSLKPEDSAIYYCALSRYQGQ |
| | | GTLVTVSS |
| 523 | F010301139 | EVQLVESGGGLVQPGGSLRLSCAASGMLFNRNTQ |
| | | GWYRQAPGKQRELVAFWFSGGYVNYNDSVKGRF |
| | | TISRDNAKRTMYLQMNSLKPEDSAIYYCSLSRYQG |
| | | QGTLVTVSS |
| 524 | F010301162 | EVQLVESGGGLVQPGGSLRLSCAASGMLFNRNTQ |
| | | GWYRQAPGKQRELVAFIFSGGWTNYNDSVKGRFTI |
| | | SRDNAKRTMYLQMNSLKPEDSAIYYCSLSRYQGQ |
| | | GTLVTVSS |
| 525 | F010301175 | EVQLVESGGGLVQPGGSLRLSCAASGMLFNRNTQ |
| | | GWYRQAPGKQRELVAFIFSGGYTTYVDSVKGRFTI |
| | | SRDNAKRTMYLQMNSLKPEDSAIYYCSLSRYLGQG |
| | | TLVTVSS |
| 526 | F010301188 | EVQLVESGGGLVQPGGSLRLSCAASGMLFNRNTQ |
| | | GWYRQAPGKQRELVAFIFSGGYTNYNDSVKGRFTI |
| | | SRDNAKRTMYLQMNSLKPEDSAIYYCSLSRYQGQ |
| | | GTLVTVSS |
| 527 | F010301191 | EVQLVESGGGLVQPGGSLRLSCAASGMLFNRNTQ |
| | | GWYRQAPGKQRELVAFIFSGGWTNYNDSVKGRFTI |
| | | SRDNAKRTMYLQMNSLKPEDSAIYYCALSVYQGQ |
| | | GTLVTVSS |
| 528 | F010301232 | EVQLVESGGGLVQPGGSLRLSCAASGMLFNRNTQ |
| | | GWYRQAPGKQRELVAFIFSGGYTTYVDSVKGRFTI |
| | | SRDNAKRTMYLQMNSLKPEDSAIYYCALSRYQGQ |
| | | GTLVTVSS |
| 529 | F010301458 | EVQLVESGGGLVQPGGSLRLSCAASGMLFNRNTQ |
| | | GWYRQAPGKQRELVAFIFSGGWTNYNDSVKGRFTI |
| | | SRDNAKRTMYLQMNSLKPEDSAIYYCALSRYQGQ |
| | | GTLVTVSS |
| 530 | F010301463 | EVQLVESGGGLVQPGGSLRLSCAASGMLFNRNTQ |
| | | GWYRQAPGKQRELVAFIFSGGYTNYNDSVKGRFTI |
| | | SRDNAKRTMYLQMNSLKPEDSAIYYCALSRYQGQ |
| | | GTLVTVSS |
| 531 | F010301301 | EVQLVESGGGLVQAGGSLRLSCDASGRILRTGYMR |
| | | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSS |
| 532 | F010301304 | EVQLVESGGGLVQAGGSLRLSCDASGRILRWGYM |
| | | RWHRQGAGKQREFVARITDDSATDYADSVKGRFTI |
| | | SRDNAKNTVYLQMNNLNPEDTAVYYCEALVTASV |
| | | RGGSIHSGTYWGRGTLVTVSS |
| 533 | F010301309 | EVQLVESGGGLVQAGGSLRLSCDASGRIVRIGYMR |
| | | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSS |
| 534 | F010301313 | EVQLVESGGGLVQAGGSLRLSCDASGRILRIGYMK |
| | | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSS |
| 535 | F010301314 | EVQLVESGGGLVQAGGSLRLSCAASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIHSGTYWGRGTLVTVSS |
| 536 | F010301328 | EVQLVESGGGLVQAGGSLRLSCDASGRILRIGYMR |
| | | WHRQGAGKQREFVARITDDSAVDYADSVKGRFTIS |
| | | RDNAKNTVYLQMNNLNPEDTAVYYCEALVTASVR |
| | | GGSIHSGTYWGRGTLVTVSS |
| 537 | F010301335 | EVQLVESGGGLVQAGGSLRLSCDASGRILRIGYMR |
| | | WHRQGAGKQREFVAVITDDSATDYADSVKGRFTIS |
| | | RDNAKNTVYLQMNNLNPEDTAVYYCEALVTASVR |
| | | GGSIHSGTYWGRGTLVTVSS |
| 538 | F010301344 | EVQLVESGGGLVQAGGSLRLSCDASGRILRIGYMR |
| | | WHRQGAGKQREFVARITDGSATDYADSVKGRFTIS |
| | | RDNAKNTVYLQMNNLNPEDTAVYYCEALVTASVR |
| | | GGSIHSGTYWGRGTLVTVSS |
| 539 | F010301346 | EVQLVESGGGLVQAGGSLRLSCDASGRILRIGYMR |
| | | WHRQGAGKQREFVARITDDSATGYADSVKGRFTIS |
| | | RDNAKNTVYLQMNNLNPEDTAVYYCEALVTASVR |
| | | GGSIHSGTYWGRGTLVTVSS |
| 540 | F010301350 | EVQLVESGGGLVQAGGSLRLSCDASGRILRIGYMR |
| | | WHRQGAGKQREFVARITDDSATVYADSVKGRFTIS |
| | | RDNAKNTVYLQMNNLNPEDTAVYYCEALVTASVR |
| | | GGSIHSGTYWGRGTLVTVSS |
| 541 | F010301360 | EVQLVESGGGLVQAGGSLRLSCDASGRILRIGYMR |
| | | WHRQGAGKQREFVARITDDSTTDYADSVKGRFTIS |
| | | RDNAKNTVYLQMNNLNPEDTAVYYCEALVTASVR |
| | | GGSIHSGTYWGRGTLVTVSS |
| 542 | F010301367 | EVQLVESGGGLVQAGGSLRLSCDASGRILRIGYMR |
| | | WHRQGAGKQREFVARITGDSATDYADSVKGRFTIS |
| | | RDNAKNTVYLQMNNLNPEDTAVYYCEALVTASVR |
| | | GGSIHSGTYWGRGTLVTVSS |
| 543 | F010301372 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGGIHSGTYWGRGTLVTVSS |
| 544 | F010301387 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASAR |
| | | GGSIHSGTYWGRGTLVTVSS |
| 545 | F010301409 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIVSGTYWGRGTLVTVSS |
| 546 | F010301416 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIHSGQYWGRGTLVTVSS |
| 547 | F010301418 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIHSGTWWGRGTLVTVSS |
| 548 | F010301425 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | VGSIHSGTYWGRGTLVTVSS |
| 549 | F010301440 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGSIHSTTYWGRGTLVTVSS |
| 550 | F010301445 | EVQLVESGGGLVQAGGSLRLSCDASGRILRIGYMR |
| | | WHRQGAGKQREFVARITDDSATDYADSVKGRFTIS |
| | | RDNAKNTVYLQMNNLNPEDTAVYYCEALVTASVR |
| | | GGYIHSGTYWGRGTLVTVSS |
| 551 | F010301557 | EVQLVESGGGLVQAGGSLRLSCAASGRILRIGYMR |
| | | WHRQGAGKQREFVARITGGSATDYADSVKGRFTIS |
| | | RDNAKNTVYLQMNNLNPEDTAVYYCEALVTASVR |
| | | GGSIHSGTYWGRGTLVTVSS |
| 552 | F010301558 | EVQLVESGGGLVQAGGSLRLSCAASGRILRIGYMR |
| | | WHRQGAGKQREFVARITGDSATGYADSVKGRFTIS |
| | | RDNAKNTVYLQMNNLNPEDTAVYYCEALVTASVR |
| | | GGSIHSGTYWGRGTLVTVSS |
| 553 | F010301559 | EVQLVESGGGLVQAGGSLRLSCAASGRILRIGYMR |
| | | WHRQGAGKQREFVARITDGSATGYADSVKGRFTIS |
| | | RDNAKNTVYLQMNNLNPEDTAVYYCEALVTASVR |
| | | GGSIHSGTYWGRGTLVTVSS |
| 554 | F010301560 | EVQLVESGGGLVQAGGSLRLSCDASGRILRIGYMR |
| | | WHRQGAGKQREFVARITGGSATGYADSVKGRFTIS |
| | | RDNAKNTVYLQMNNLNPEDTAVYYCEALVTASVR |
| | | GGSIHSGTYWGRGTLVTVSS |
| 555 | F010301561 | EVQLVESGGGLVQAGGSLRLSCAASGRILRIGYMR |
| | | WHRQGAGKQREFVARITGDSATDYADSVKGRFTIS |
| | | RDNAKNTVYLQMNNLNPEDTAVYYCEALVTASVR |
| | | GGSIHSGTYWGRGTLVTVSS |
| 556 | F010301562 | EVQLVESGGGLVQAGGSLRLSCAASGRILRIGYMR |
| | | WHRQGAGKQREFVARITDGSATDYADSVKGRFTIS |
| | | RDNAKNTVYLQMNNLNPEDTAVYYCEALVTASVR |
| | | GGSIHSGTYWGRGTLVTVSS |
| 557 | F010301564 | EVQLVESGGGLVQAGGSLRLSCDASGRILRIGYMR |
| | | WHRQGAGKQREFVARITGGSATDYADSVKGRFTIS |
| | | RDNAKNTVYLQMNNLNPEDTAVYYCEALVTASVR |
| | | GGSIHSGTYWGRGTLVTVSS |
| 558 | F010301565 | EVQLVESGGGLVQAGGSLRLSCDASGRILRIGYMR |
| | | WHRQGAGKQREFVARITGDSATGYADSVKGRFTIS |
| | | RDNAKNTVYLQMNNLNPEDTAVYYCEALVTASVR |
| | | GGSIHSGTYWGRGTLVTVSS |
| 559 | F010301566 | EVQLVESGGGLVQAGGSLRLSCDASGRILRIGYMR |
| | | WHRQGAGKQREFVARITDGSATGYADSVKGRFTIS |
| | | RDNAKNTVYLQMNNLNPEDTAVYYCEALVTASVR |
| | | GGSIHSGTYWGRGTLVTVSS |
| 560 | F010301567 | EVQLVESGGGLVQAGGSLRLSCAASVRPYSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 561 | F010301568 | EVQLVESGGGLVQAGGSLRLSCAASVRPFSTSAMT |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 562 | F010301574 | EVQLVESGGGLVQAGGSLRLSCAASVRPFSTSAMA |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 563 | F010301578 | EVQLVESGGGLVQAGGSLRLSCAASVRPFGTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 564 | F010301579 | EVQLVESGGGLVQAGGSLRLSCAASVKPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 565 | F010301580 | EVQLVESGGGLVQAGGSLRLSCAASVTPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEYWGQGTLVTVSS |
| 566 | F010301584 | EVQLVESGGGLVQAGGSLRLSCAASVRPFSTSAMG |
| | | WFRQAPEKEREAVAGILTNGIVTYYADSVKGRFTIS |
| | | RDNAKNEVYLQMNKLKPEDTAVYYCALDRDYGG |
| | | RSFSAYEYEYWGQGTLVTVSS |
| 567 | F010301585 | EVQLVESGGGLVQAGGSLRLSCAASVRPFSTSAMG |
| | | WFRQAPEKEREAVAGILWNGIPTYYADSVKGRFTI |
| | | SRDNAKNEVYLQMNKLKPEDTAVYYCALDRDYG |
| | | GRSFSAYEYEYWGQGTLVTVSS |
| 568 | F010301586 | EVQLVESGGGLVQAGGSLRLSCAASVRPFSTSAMG |
| | | WFRQAPEKEREAVAGILANGIVTYYADSVKGRFTI |
| | | SRDNAKNEVYLQMNKLKPEDTAVYYCALDRDYG |
| | | GRSFSAYEYEYWGQGTLVTVSS |
| 569 | F010301589 | EVQLVESGGGLVQAGGSLRLSCAASVRPFSTSAMG |
| | | WFRQAPEKEREAVAGILWNGPVTYYADSVKGRFTI |
| | | SRDNAKNEVYLQMNKLKPEDTAVYYCALDRDYG |
| | | GRSFSAYEYEYWGQGTLVTVSS |
| 570 | F010301591 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYK |
| | | GRSFSAYEYEYWGQGTLVTVSS |
| 571 | F010301592 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GSSFSAYEYEYWGQGTLVTVSS |
| 572 | F010301593 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDKDYG |
| | | GRSFSAYEYEYWGQGTLVTVSS |
| 573 | F010301594 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GKSFSAYEYEYWGQGTLVTVSS |
| 574 | F010301595 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRAYG |
| | | GRSFSAYEYEYWGQGTLVTVSS |
| 575 | F010301596 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYA |
| | | GRSFSAYEYEYWGQGTLVTVSS |
| 576 | F010301598 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYQ |
| | | GRSFSAYEYEYWGQGTLVTVSS |
| 577 | F010301604 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GQSFSAYEYEYWGQGTLVTVSS |
| 578 | F010301606 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAKEYEYWGQGTLVTVSS |
| 579 | F010301607 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAAEYEYWGQGTLVTVSS |
| 580 | F010301609 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAQEYEYWGQGTLVTVSS |
| 581 | F010301612 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSATEYEYWGQGTLVTVSS |
| 582 | F010301617 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSQSAYEYEYWGQGTLVTVSS |
| 583 | F010301618 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAYEYEHWGQGTLVTVSS |
| 584 | F010301619 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSKSAYEYEYWGQGTLVTVSS |
| 585 | F010301621 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSAGEYEYWGQGTLVTVSS |
| 586 | F010301622 | EVQLVESGGGLVQAGGSLRLSCAASVRPFSTSAMG |
| | | WFRQAPEKEREAVAGILWNGIVTYYADSVKGRFTI |
| | | SRDNAKNEVYLQMNKLKPEDTAVYYCALDRDYG |
| | | GRSFSADEYEYWGQGTLVTVSS |
| 587 | F010301627 | EVQLVESGGGLVQAGGSLRLSCAASVRPFSTSAMG |
| | | WFRQAPEKEREAVAAILWNGIVTYYADSVKGRFTI |
| | | SRDNAKNEVYLQMNKLKPEDTAVYYCALDRDYG |
| | | GRSFSAYEYEYWGQGTLVTVSS |
| 588 | F010301629 | EVQLVESGGGLVQAGGSLRLSCAASVRPFSTSAMG |
| | | WFRQAPEKEREAVASILWNGIVTYYADSVKGRFTI |
| | | SRDNAKNEVYLQMNKLKPEDTAVYYCALDRDYG |
| | | GRSFSAYEYEYWGQGTLVTVSS |
| 589 | F010300316 | EVQLVESGGGLVQPGGSLRLSCAASGSIFNINSMA |
| | | WYRRAPGKQRELVASSTNGGSWNYADSVKGRFTI |
| | | SRDNAKRVYLQMNSLTPEDTAVYYCNALLQPSIYD |
| | | ISRTYWGQGTLVTVSS |
| 590 | F010300468 | EVQLVESGGGLVQPGGSLRLSCAASGSIFNINSMA |
| | | WYRRAPGKQRELVASSTNGGSTNYADSVKGRFTIS |
| | | RDNAKRVYLQMNSLTPEDTAVYYCRALLQPSIYDI |
| | | SRTYWGQGTLVTVSS |
| 591 | F010300477 | EVQLVESGGGLVQPGGSLRLSCAASGSIFNINSMA |
| | | WYRRAPGKQRELVASSTNGGSTNYADSVKGRFTIS |
| | | RDNAKRVYLQMNSLTPEDTAVYYCNWLLQPSIYDI |
| | | SRTYWGQGTLVTVSS |
| 592 | F010300631 | EVQLVESGGGLVQPGGSLRLSCAASGPVFNINSMA |
| | | WYRRAPGKQRELVAYSTPGGSTNYADSVKGRFTIS |
| | | RDNAKRVYLQMNSLTPEDTAVYYCRALLQPSIYDI |
| | | SRTYWGQGTLVTVSS |
| 593 | F010300659 | EVQLVESGGGLVQPGGSLRLSCAASGPVFNINSMA |
| | | WYRRAPGKQRELVAYSTPGWDWNYADSVKGRFTI |
| | | SRDNAKRVYLQMNSLTPEDTAVYYCRWLLQPSIY |
| | | DISRTYWGQGTLVTVSS |
| 594 | F010300684 | EVQLVESGGGLVQPGGSLRLSCAASGPVFNWNSM |
| | | AWYRRAPGKQRELVASSTPGGSTNYADSVKGRFTI |
| | | SRDNAKRVYLQMNSLTPEDTAVYYCRALLQPSIYD |
| | | ISRIYWGQGTLVTVSS |
| 595 | F010300796 | EVQLVESGGGLVQPGGSLRLSCAASGPVFNINRMA |
| | | WYRRAPGKQRELVAYSTPGGDTNYADSVKGRFTIS |
| | | RDNAKRVYLQMNSLTPEDTAVYYCNALLQPSIYDI |
| | | SRTYWGQGTLVTVSS |
| 596 | F010300880 | EVQLVESGGGLVQPGGSLRLSCAASGPVFNINRMA |
| | | WYRRAPGKQRELVASSTPGGDTNYADSVKGRFTIS |
| | | RDNAKRVYLQMNSLTPEDTAVYYCRWLLQPSIYDI |
| | | SRTYWGQGTLVTVSS |
| 597 | F010300900 | EVQLVESGGGLVQPGGSLRLSCAASGPVFNINRMA |
| | | WYRRAPGKQRELVASSTPGGDTNYADSVKGRFTIS |
| | | RDNAKRVYLQMNSLTPEDTAVYYCRWLLQPSIYDI |
| | | SRIYWGQGTLVTVSS |
| 598 | F010300948 | EVQLVESGGGLVQPGGSLRLSCAASGPVFNINRMA |
| | | WYRRAPGKQRELVASSTPGGSTNYADSVKGRFTIS |
| | | RDNAKRVYLQMNSLTPEDTAVYYCRWLLQPSIYDI |
| | | SRIYWGQGTLVTVSS |
| 599 | F010300990 | EVQLVESGGGLVQPGGSLRLSCAASGPVFNINRMA |
| | | WYRRAPGKQRELVAYSTPGGSTNYADSVKGRFTIS |
| | | RDNAKRVYLQMNSLTPEDTAVYYCNALLQPSIYDI |
| | | SRIYWGQGTLVTVSS |
| 600 | F010301000 | EVQLVESGGGLVQPGGSLRLSCAASGPVFNINRMA |
| | | WYRRAPGKQRELVAYSTPGGDTNYADSVKGRFTIS |
| | | RDNAKRVYLQMNSLTPEDTAVYYCNALLQPSIYDI |
| | | SRIYWGQGTLVTVSS |
| 601 | F010301459 | EVQLVESGGGLVQPGGSLRLSCAASGPVFNINRMA |
| | | WYRRAPGKQRELVASSTPGGDTNYADSVKGRFTIS |
| | | RDNAKRVYLQMNSLTPEDTAVYYCRALLQPSIYDI |
| | | SRTYWGQGTLVTVSS |
| 602 | F010301460 | EVQLVESGGGLVQPGGSLRLSCAASGPVFNINRMA |
| | | WYRRAPGKQRELVAYSTPGGSTNYADSVKGRFTIS |
| | | RDNAKRVYLQMNSLTPEDTAVYYCRALLQPSIYDI |
| | | SRTYWGQGTLVTVSS |
| 603 | F010301462 | EVQLVESGGGLVQPGGSLRLSCAASGPVFNINRMA |
| | | WYRRAPGKQRELVAYSTPGGDTNYADSVKGRFTIS |
| | | RDNAKRVYLQMNSLTPEDTAVYYCRALLQPSIYDI |
| | | SRTYWGQGTLVTVSS |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A Nav1.7 binder that binds to a human voltage-gated sodium channel Nav1.7α protein subunit (human NaV1.7α subunit) between amino acids 272 and 331 of the human NaV1.7α subunit Domain 1 S5-S6 loop, wherein the human NaV1.7α subunit comprises the amino acid sequence set forth in SEQ ID NO: 1.

2. The Nav1.7 binder of claim 1, wherein the Nav1.7 binder contacts amino acids F276, R277, E281, and V331 of the human NaV1.7α subunit.

3. The Nav1.7 binder of claim 2, wherein Nav1.7 binder binds to the human NaV1.7α subunit comprising one or more mutations at residue F276, R277, E281 and/or V331 with lower affinity than to human NaV1.7 alpha subunit lacking such mutations.

4. The Nav1.7 binder of claim 1, wherein the Nav1.7 binder further is capable of binding a rhesus monkey human NaV1.7α subunit with a lower affinity than it binds to the human NaV1.7α subunit.

5. The Nav1.7 binder of claim 1, wherein the Nav1.7 binder is an antigen-binding fragment of either an antibody or a heavy chain antibody.

6. The Nav1.7 binder of claim 1, wherein the Nav1.7 binder is an immunoglobulin single variable domain (ISVD).

7. The Nav1.7 binder of claim 6, wherein the Nav1.7 binder comprises:
(a) a complementarity determining region (CDR) 1 comprising the amino acid sequence set forth in SEQ ID NO: 247, a CDR2 comprising the amino acid sequence set forth in SEQ ID NO: 248, and a CDR3 comprising the amino acid sequence set forth in SEQ ID NO: 249; or
(b) a CDR1 comprising the amino acid sequence set forth in SEQ ID NO: 250, a CDR2 comprising the amino acid sequence set forth in SEQ ID NO: 251, and a CDR3 comprising the amino acid sequence set forth in SEQ ID NO: 252; or
(c) a CDR1 comprising the amino acid sequence set forth in SEQ ID NO: 253, a CDR2 comprising the amino acid sequence set forth in SEQ ID NO: 254, and a CDR3 comprising the amino acid sequence SRY; or
(d) a CDR1 comprising the amino acid sequence set forth in SEQ ID NO: 256, a CDR2 comprising the amino acid sequence set forth in SEQ ID NO: 257, and a CDR3 comprising the amino acid sequence set forth in SEQ ID NO: 258; or
(e) a CDR1 comprising the amino acid sequence set forth in SEQ ID NO: 259, a CDR2 comprising the amino acid sequence set forth in SEQ ID NO: 260, and a CDR3 comprising the amino acid sequence set forth in SEQ ID NO: 261; or
(f) a CDR1 comprising the amino acid sequence set forth in SEQ ID NO: 262, a CDR2 comprising the amino acid sequence set forth in SEQ ID NO: 263, and a CDR3 comprising the amino acid sequence set forth in SEQ ID NO: 264; or
(g) a CDR1 comprising the amino acid sequence set forth in SEQ ID NO: 196, a CDR2 comprising the amino acid sequence set forth in SEQ ID NO: 198, and a CDR3 comprising the amino acid sequence set forth in SEQ ID NO: 200; or
(h) a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 206; or
(i) a CDR1 comprising the amino acid sequence set forth in SEQ ID NO: 207, a CDR2 comprising the amino acid sequence set forth in SEQ ID NO: 213, and a CDR3 comprising the amino acid sequence set forth in SEQ ID NO: 219; or
(j) a CDR1 comprising the amino acid sequence set forth in SEQ ID NO: 221, a CDR2 comprising the amino acid sequence set forth in SEQ ID NO: 223, and a CDR3 comprising the amino acid sequence set forth in SEQ ID NO: 225.

8. The Nav1.7 binder of claim 6, wherein the Nav1.7 binder comprises:
(a) a CDR1 comprising the amino acid sequence set forth in SEQ ID NO: 196 or SEQ ID NO: 197; a CDR2 comprising the amino acid sequence set forth in SEQ ID NO: 198 or SEQ ID NO: 199; and, a CDR3 comprising the amino acid sequence set forth in SEQ ID NO: 200; or
(b) a CDR1 comprising the amino acid sequence set forth in SEQ ID NO: 201; a CDR2 comprising the amino acid sequence set forth in SEQ ID NO: 202, SEQ ID NO: 203, SEQ ID NO: 204, or SEQ ID NO: 205; and, a CDR3 comprising the amino acid sequence set forth in SEQ ID NO: 206; or
(c) a CDR1 comprising the amino acid sequence set forth in SEQ ID NO: 207, SEQ ID NO: 208, SEQ ID NO: 209, SEQ ID NO: 210, SEQ ID NO: 211, or SEQ ID NO: 212; a CDR2 comprising the amino acid sequence set forth in SEQ ID NO: 213, SEQ ID NO: 214, SEQ ID NO: 215, SEQ ID NO: 216, SEQ ID NO: 217, or SEQ ID NO: 218; and a CDR3 comprising the amino acid sequence set forth in SEQ ID NO: 219; or
(d) a CDR1 comprising the amino acid sequence set forth in SEQ ID NO: 201 or SEQ ID NO: 222; a CDR2 comprising the amino acid sequence set forth in SEQ ID NO: 223 or SEQ ID NO: 224; and a CDR3 comprising the amino acid sequence set forth in SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, or SEQ ID NO: 233; or
(e) a CDR1 comprising the amino acid sequence set forth in SEQ ID NO: 201; a CDR2 comprising the amino acid sequence set forth in SEQ ID NO: 205; and, a CDR3 comprising the amino acid sequence set forth in SEQ ID NO: 206; or
(f) a CDR1 comprising the amino acid sequence set forth in SEQ ID NO: 211; a CDR2 comprising the amino acid sequence set forth in SEQ ID NO: 215; and, a CDR3 comprising the amino acid sequence set forth in SEQ ID NO: 219; or
(g) a CDR1 comprising the amino acid sequence set forth in SEQ ID NO: 222; a CDR2 comprising the amino acid sequence set forth in SEQ ID NO: 223; and, a CDR3 comprising the amino acid sequence set forth in SEQ ID NO: 233.

9. The Nav1.7 binder of claim 6, wherein the Nav1.7 binder comprises:
(a) an amino acid sequence selected from the group consisting of SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55;
(b) an amino acid sequence selected from the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81; or
(c) an amino acid sequence selected from the group consisting of SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, and SEQ ID NO: 97; or
(d) an amino acid sequence selected from the group consisting of SEQ ID NO: 98, SEQ ID NO: 99, SEQ ID NO: 100, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, and SEQ ID NO: 153; or
(e) an amino acid sequence selected from the group consisting of SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, and SEQ ID NO: 195.

10-16. (canceled)

17. A composition comprising a Nav1.7 binder of claim 1, and a pharmaceutically acceptable carrier.

18. A method for treating an individual with chronic pain comprising:
administering to the individual a therapeutically effective amount of the Nav1.7 binder of claim 1 to treat the chronic pain.

19-24. (canceled)

25. A Navβ1 binder comprising:
(a) a first immunoglobulin single variable domain (ISVD) comprising three complementarity determining regions (CDRs) wherein CDR1 comprises the amino acid sequence set forth in SEQ ID NO: 425, CDR2 comprises the amino acid sequence set forth in SEQ ID NO: 426, and CDR3 comprises the amino acid sequence set forth in SEQ ID NO: 427; or,
(b) a second ISVD comprising three CDRs wherein CDR1 comprises the amino acid sequence set forth in SEQ ID NO: 437, CDR2 comprises the amino acid sequence set forth in SEQ ID NO: 438, and CDR3 comprise the amino acid sequence set forth in SEQ ID NO: 439.

26. The Navβ1 binder of claim 25, wherein the first ISVD comprises the amino acid sequence set forth in SEQ ID NO: 411 and the second ISVD comprises the amino acid sequence set forth in SEQ ID NO: 415.

27-30. (canceled)

31. A Navβ2 binder comprising:
(a) a first immunoglobulin single variable domain (ISVD) comprising three complementarity determining regions (CDRs) wherein CDR1 comprises the amino acid sequence set forth in SEQ ID NO: 422, a CDR2 comprising the amino acid sequence set forth in SEQ ID NO: 423, and a CDR3 comprising the amino acid sequence set forth in SEQ ID NO: 424;
(b) a second ISVD comprising three CDRs wherein CDR1 comprises the amino acid sequence set forth in SEQ ID NO: 428, a CDR2 comprising the amino acid sequence set forth in SEQ ID NO: 429, and a CDR3 comprising the amino acid sequence set forth in SEQ ID NO: 430;
(c) a third ISVD comprising three CDRs wherein CDR1 comprises the amino acid sequence set forth in SEQ ID NO: 431, a CDR2 comprising the amino acid sequence set forth in SEQ ID NO: 432, and a CDR3 comprising the amino acid sequence set forth in SEQ ID NO: 433; or
(d) a fourth ISVD comprising three CDRs wherein CDR1 comprises the amino acid sequence set forth in SEQ ID NO: 434, a CDR2 comprising the amino acid sequence set forth in SEQ ID NO: 435, and a CDR3 comprising the amino acid sequence set forth in SEQ ID NO: 436.

32-36. (canceled)

37. A Nav1.7-Navβ bispecific binder comprising a Nav1.7 binder of claim 1 and a Navβ binder selected from the group consisting of the Navβ1 binder of claim 25 and the Navβ2 binder of claim 31.

38-45. (canceled)
